(19) United States
(12) Patent Application Publication
(10) Pub. No.: US 2024/0300980 A1
(43) Pub. Date: Sep. 12, 2024

(54) HETEROCYCLIC COMPOUNDS AS KRAS INHIBITOR, AND PREPARATION THEREFOR AND USE THEREOF IN TREATMENT

(71) Applicant: SHANGHAI KECHOW PHARMA, INC., Shanghai (CN)

(72) Inventors: Hongqi Tian, Shanghai (CN); Gongchao Huang, Shanghai (CN); Xuguang Gao, Shanghai (CN); Huanhuan Liu, Shanghai (CN); Xingkai Wang, Shanghai (CN)

(21) Appl. No.: 18/563,357

(22) PCT Filed: May 22, 2022
(86) PCT No.: PCT/CN2022/094300

§ 371 Date: Nov. 21, 2023

(30) Foreign Application Priority Data
May 22, 2021 (CN) 202110561238.1
May 10, 2022 (CN) 202210508194.0

(51) Int. Cl. C07D 519/00; A61K 31/4995; A61K 31/517; A61K 31/519; A61K 31/5386; A61K 31/547; A61P 35/00; C07D 487/04; C07D 487/08

(52) U.S. Cl. CPC C07D 519/00; A61K 31/4995; A61K 31/517; A61K 31/519; A61K 31/5386; A61K 31/547; A61P 35/00; C07D 487/04; C07D 487/08

(57) ABSTRACT

Disclosed is a compound of formula (I) and (II) or a pharmaceutically acceptable salt, prodrug, tautomer or stereoisomer, and solvate thereof. The compound can be applied to treatment of cancers and inflammations of mammals. Further disclosed are a preparation method for the compound of formula (I) and (II) and a pharmaceutical composition containing the compound.

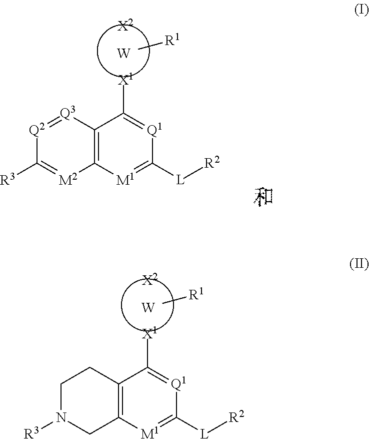

### TECHNICAL FIELD

The present invention relates to some novel heterocyclic compounds or pharmaceutically acceptable salts thereof, which can be used for the treatment or prevention of cancers associated with H-ras, K-ras, or N-ras inhibition. The present invention further relates to pharmaceutical compositions comprising such compounds and salts thereof, intermediates in the preparation of the compounds, and methods for treating cancers associated with H-ras, K-ras, or N-ras inhibition by using the compounds or pharmaceutically acceptable salts thereof.

### BACKGROUND ART

In 1982, Weinberg and Barbacid isolated, for the first time, a transforming gene from a human bladder cancer cell line, which could make NIH 3T3 cells undergo malignant transformation; whereas, DNA extracted from normal human tissues had no such effects. Subsequently, Santos and Parada found that the above-mentioned transforming gene was not a new gene, but a human homologous gene of the ras gene of Harvery murine sarcoma virus, named H2ras. In the same year, Krontiris found a homolog of Kirsten murine sarcoma virus gene in human lung cancer cells, named K-ras. Another similar gene, called N2ras, was a gene similar to ras and was found when human neuroblastoma DNA infected NIH 3T3 cells. This gene is irrelevant to viruses.

Ras gene is quite conservative in evolution and widely exists in various eukaryotes such as mammals, fruit flies, fungi, nematodes, and yeasts, suggesting that it has important physiological functions. The mammalian ras gene family has three members, i.e., H-ras, K-ras, and N-ras, wherein the fourth exon of K-ras has two variants, A and B. Various ras genes have similar structures, all of which are composed of four exons, which are distributed on DNA with a full length of about 30 kb. The encoded product thereof is a protein with a relative molecular weight of 21,000 and is thus called P21 protein. It has been demonstrated that H-ras is located on the short arm of human chromosome 11 (11p15.1-p15.3), K-ras is located on the short arm of human chromosome 12 (12p1.1-pter), and N-ras is located on the short arm of human chromosome 1 (1p22-p32). Except for the variation of the fourth exon of K-ras, the P21-encoding sequence of each ras gene is evenly distributed on four exons, and the sequence and size of introns are very different, so the whole gene is also very different. For example, human K-ras is 35 kb long and N-ras is 3 kb long. Since there are two exons 4, K-ras can be spliced in two ways, but the content of mRNA encoding K-ras-B is high. Except K-ras-B which contains 188 amino acids, the other two ras proteins both contain 189 amino acids.

Ras(P21) protein, which is located on the inner side of the cell membrane, plays an important role in transmitting cell growth and differentiation signals. It belongs to guanosine triphosphate (GTP) binding protein (a coupling factor of cell information transmission), which regulates information transmission by mutual transformation between GTP and guanosine diphosphate (GDP). P21 has a strong affinity with GTP and GDP and a weak GTPase activity. Under normal circumstances, the binding between P21 and GDP is in an inactive state. When the growth differentiation factor outside the cell transmits a signal to P21 on the inner side of the cell membrane, the activity of binding P21 to GTP can be enhanced, making the binding of P21 and GTP be in an activated state, causing the signal system to be open. Due to the GTPase activity, P21 can hydrolyze GTP into GDP. After P21 is bound with GDP, P21 becomes inactivated and the signal system is closed. Under normal circumstances, the GTPase activity of P21 is very weak. After it is bound with GTPase-activating protein (GAP), the hydrolysis rate thereof can be increased by 10,000 times, causing P21 to be deactivated. After P21 is bound with GDP, guanosine nucleotide-releasing protein (GNRP) can be activated, and GNRP makes P21 release GDP and bind GTP. Therefore, by the mutual transformation between GTP and GDP, P21 can be regulated to turn on and off the signal system in a controlled way, thereby completing the process of transmitting growth and differentiation signals into cells.

More than ⅕ of cancer patients are accompanied by ras gene mutations, which mostly occur in G12, G13, and Q61 residues. The mutations lead to GAP protein mediation failure and the ras signal is in a continuously activated state.

The RAS gene family is the most common mutant gene in human cancer. RAS mutations are found in 90% of pancreatic cancers, 45% of colon cancers, and 35% of lung cancers. Among the three Ras genes, Kirsten-RAS (KRAS) is the most common subtype of mutation, accounting for 86%. The other two subtypes of neuroblastoma-RAS (NRAS) and Harvey-RAS (HRAS) have low mutation rates (11% and 3%). KRAS protein has the following mutations. KRAS-G12C mutations play a dominant role in NSCLC (about 45-50% of mutant KRAS-G12C). On the other hand, KRAS-G12D (in which glycine mutation at codon-12 is aspartic acid) is very important in pancreatic cancer (61%), colon cancer (42%) and NSCLC (22%).

Drug research on the treatment of KRAS-G12C mutations has achieved exciting results, and the clinical results of AMG510 and MRTX849 give cancer patients hope. However, there is no corresponding targeting drug for KRAS G12D mutation. At the 32nd MOLECULAR TARGETS AND CANCER THERAPEUTICS SYMPOSIUM (International Symposium on Molecular Targets and Cancer Therapeutics), Mirati Therapeutics announced the research progress of its KRAS-G12D selective inhibitor MRTX1133, which is currently in the preclinical stage. Therefore, new KRAS-G12D inhibitors need to be developed.

The present invention designs and synthesizes a range of chemical molecules, which have strong biological activity of inhibiting ras, and provides a method for treating related cancers by inhibiting H-ras, K-ras, or N-ras.

### SUMMARY OF THE INVENTION

The present invention provides compounds capable of regulating G12C mutant KRAS, HRAS, and/or NRAS proteins, including stereoisomers, pharmaceutically acceptable salts, tautomers, and prodrugs thereof. Further provided is a method of using such compounds to treat various diseases or conditions, such as cancer.

In one aspect of the present invention, there is provided a compound of formula (I) or formula (II) or a pharmaceutically acceptable salt, solvate, tautomer, stereoisomer or prodrug thereof, wherein the compounds of formula (I) and formula (II) are:

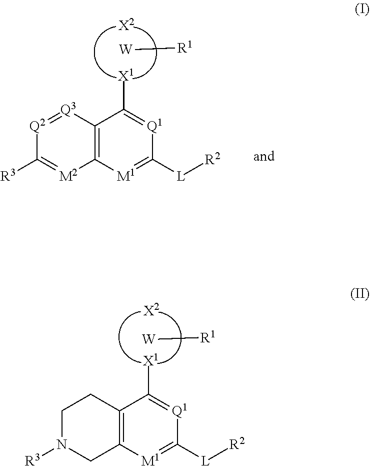

wherein:

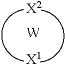

is a 4- to 12-membered saturated or partially saturated monocyclic, bridged cyclic, or spirocyclic ring or —X¹(R^{1a}R^{1b})C₁₋₄X², wherein the saturated or partially saturated monocyclic ring is optionally additionally substituted with one or more R¹, X¹ is selected from N and CR⁴; X² is selected from NR⁴, CR⁴ and S(O)_{0,1,2}R⁴,
wherein,
R1 is selected from hydrogen, halogen, C₁₋₆ alkyl optionally substituted by halogen or hydroxyl or —NR^{1a}R^{1b}, CN, —OR^{1a}, —SR^{1a}, —NR^{1a}R^{1b}, —S(O)R^{1a}, —S(O)₂R^{1a}, —C(O)R^{1a}, —C(O)OR^{1a}, —NR^{1a}C(O)R^{1b}, —C(O)NR^{1a}R^{1b}, —S(O)₂N(R^{1a}R^{1b})₂ and 5 to 6-membered heteroaryl, wherein R^{1a} and R^{1b} are each independently hydrogen, C₁₋₆ alkyl, hydroxy C₁₋₆ alkyl, halogenated C₁₋₆ alkyl and C₁₋₆ alkoxy C₁₋₆ alkyl, C₃₋₆ cycloalkyl;
R⁴ is selected from hydrogen, halogen, C₁₋₆ alkyl optionally substituted by halogen or hydroxyl, CN, —OR^{4a}, —SR^{4a}, —S(O)R^{4a}, —S(O)₂R^{4a}, —C(O)R^{4a}, —C(O)OR^{4a}, —NR^{4a}C(O)R^{4b}, —C(O)NR^{4a}R^{4b} and —S(O)₂N(R^{4a}R^{4b})₂, wherein R^{4a} and R^{4b} are each independently hydrogen, C₁₋₆ alkyl, hydroxy C₁₋₆ alkyl, halogenated C₁₋₆ alkyl and C₁₋₆ alkoxy C₁₋₆ alkyl;
L is a single bond, —O—, —S—, —NR^{La}—, —O—(CR^{La}R^{Lb})ₜ—, —S—(CR^{La}R^{Lb})ₜ—, —NR^{c}—(CR^{La}R^{Lb})ₜ—, —(CR^{La}R^{Lb})ₜ—O—, —(CR^{La}R^{Lb})ₜ—S—, —(CR^{La}R^{Lb})ₜ—NR^{Lc}—, —C(O)—, —SO₂—, —SO—, —C(O)—O—, —OC(O)—, —C(O)—NR^{La}— or —N^{Lb}C(O)—, wherein R^{La}, R^{Lb} and R^{Lc} are each independently hydrogen and C₁₋₆ alkyl, or R^{La} and R^{Lb} connected to the same carbon atom form C₃-C₆ cycloalkyl with the connected carbon atom, and t is an integer from 1 to 6;
R² is C₁₋₆ alkyl, C₂₋₆ alkenyl, C₂₋₆ alkynyl, C₃₋₈ cycloalkyl, aryl, heteroaryl, or heterocyclyl, wherein the C₁₋₆ alkyl, C₂₋₆ alkenyl, C₂₋₆ alkynyl, C₃₋₈ cycloalkyl, aryl, heteroaryl and heterocyclyl are each independently unsubstituted or substituted by one or more halogen, cyano, C₁₋₆ alkyl, halogenated C₁₋₆ alkyl, hydroxy C₁₋₆ alkyl, C₁₋₆ alkoxy C₁₋₆ alkyl, oxo, —OR^{2a}, —C(O)R^{2a}, —(CR^{2a}R^{2b})ₘ—OC(O)NR^{2c}R^{2d}, —CO₂R^{2a}, —CONR^{2c}R^{2d}, —NR^{2c}R^{2d}, —NR^{2c}R^{2d}, C₃₋₈ cycloalkyl, C₃₋₈ cycloalkyl C₁₋₆ alkyl, aryl, heteroaryl, and heterocyclyl, wherein R^{2a}, R^{2b}, R^{2c}, and R^{2d} are each independently hydrogen, C₁₋₆ alkyl, hydroxy C₁₋₆ alkyl, halogenated C₁₋₆ alkyl and C₁₋₆ alkoxy C₁₋₆ alkyl, or R^{2c} and R^{2d} together with the nitrogen atom to which they are attached, form an 4- to 6-membered heterocycle, The heterocycle contains 0, 1, or 2 additional heteroatoms selected from nitrogen, oxygen, and sulfur as ring members, and wherein m is an integer from 1 to 6;
R³ is aryl or heteroaryl, wherein the aryl or heteroaryl is optionally substituted by one or more R⁸; Each R¹ is independently halogen, cyano, oxo, C₁₋₆ alkyl optionally substituted by halogen, cyano, hydroxyl or deuterium, C₂-C₆ alkenyl optionally substituted by hydroxyl or deuterium, C₂-C₆ alkynyl optionally substituted by hydroxyl or deuterium, —OR^{8a}, —SR^{8a}, —S(O)₂R^{8a}, —P(═O)R^{8a}R^{1b}, —NR^{8a}R^{8b}, —C(O)NR^{8a}R^{1b}, optionally C₃-C₆ cycloalkyl substituted by halogen or C₁₋₆ alkyl, C₃-C₈ cycloalkyl, heterocyclyl, heteroaryl and aryl, wherein R^{8a} and R^{8b} are each independently hydrogen, C₁₋₆ alkyl substituted by halogen and C₁₋₆ alkoxy-C₁₋₆ alkyl-; or two R¹ on the same carbon atom form C₃-C₈ cycloalkyl; or R¹ on two adjacent carbon atoms together with their connected carbon atoms form C₃-C₈ cycloalkyl;
Q¹, Q², and Q³ are each independently N or CR⁶, and M¹ and M² are each independently N or CR⁷, provided that at least one of Q¹ and M¹ is N;
wherein R⁶ and R⁸ are each independently hydrogen, halogen, hydroxyl, cyano, nitro, C₁₋₆ alkyl, C₂₋₆ alkenyl, C₂₋₆ alkynyl, C₃₋₈ cycloalkyl, aryl, heteroaryl or heterocyclyl, —OR^{6a}, —C(O)R^{6a}, —CO₂R^{6a}, —CONR^{6a}R^{6b} or —NR^{6a}R^{6b}, wherein the C₁₋₆ alkyl, C₂₋₆ alkenyl, C₂₋₆ alkynyl, C₃₋₈ cycloalkyl, aryl, heteroaryl and heterocyclyl are independently substituted by one or more of oxo, halogen, hydroxyl, C₁₋₄ alkoxy, C₁₋₄ alkyl, C₃₋₆ cycloalkyl, nitro, cyano and —NRdRe, wherein R^{6a}, R^{6b}, R^{6c} and R^{6d} are each independently hydrogen, C₃₋₆ alkyl, C₁₋₆ alkyl, hydroxy C₁₋₆ alkyl, halogenated C₁₋₆ alkyl and C₁₋₆ alkoxy C₁₋₆ alkyl;
Or R⁷ on M² and substituent R¹ on R³ form a ring structure together with the part to which they are connected.

In some embodiments,

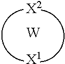

is

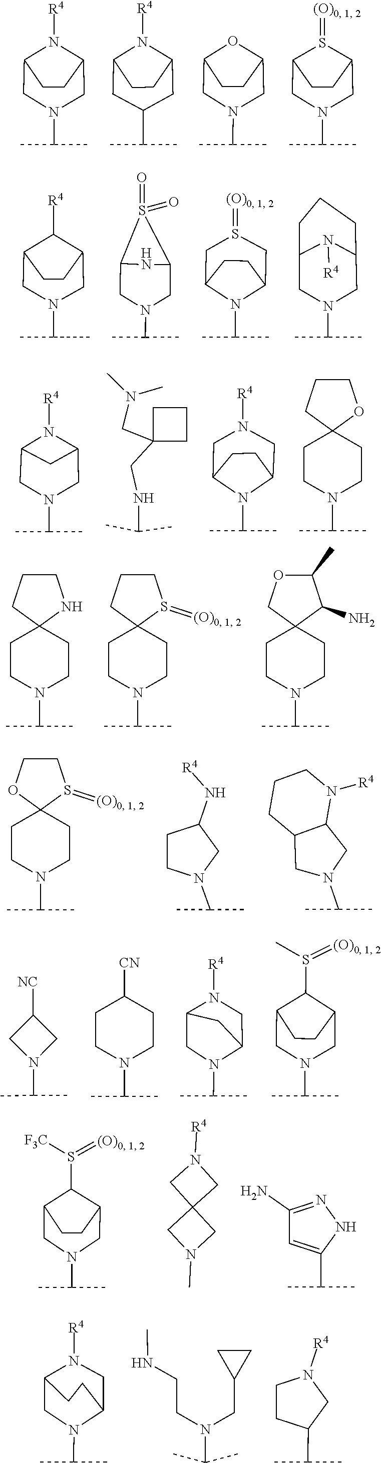

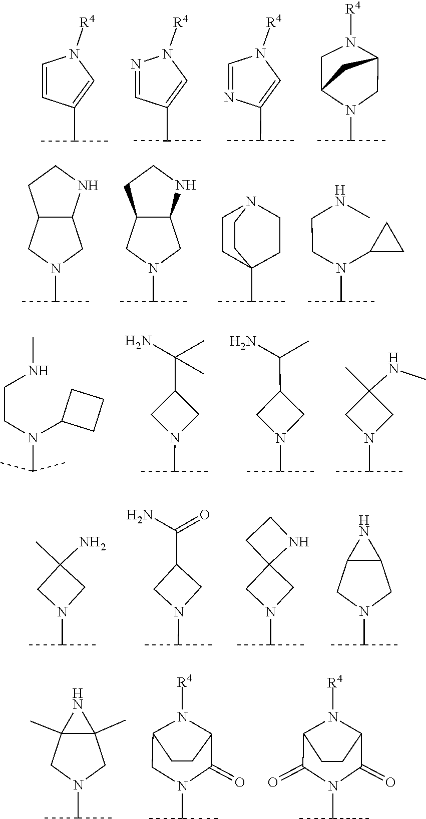

wherein R⁴ is independently hydrogen, halogen, C₁₋₆ alkyl, CN, —OR⁴, R^{4a}, S(O)R^{4a}—S(O)₂R^{4a}, —C(O)R^{4a}, —C(O)OR^{4a}, —NR^{4a}C(O)R^{4b}, —C(O)NR^{4a}R^{4b} and —S(O)₂N(R^{4a}R^{4b})₂, wherein R^{4a} and R^{4b} are independently hydrogen, C₁₋₆ alkyl and hydroxy C₁₋₆ alkyl, Preferably R⁴ is independently selected from hydrogen, —C(O)CH₂OH, —C(O)NH₂, —C(O)N(CH₃)₂, F, Br, Cl, —OH, —SCH₃, —S(O)CH₃, —S(O)₂CH₃, —S(O)₂N(CH₃)₂, —S(O)₂NH₂, CF₃ and CN.

In some embodiments,

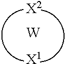

is

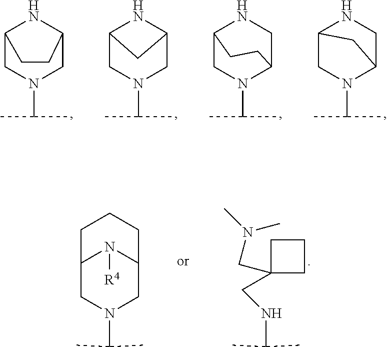

In some embodiments, L is —O—CH₂— or —O—.

In some embodiments, L is —O—CH₂—, and R² is heterocyclyl, wherein heterocyclyl is unsubstituted or substituted by one or more of halogen, C₁₋₆ alkyl, —OR^{2a} and —(CR^{2a}R²)^{m}—OC(O)NR^{2c}R^{2a}, wherein each variable as defined for formula (I); preferably, the heterocyclyl is unsubstituted or substituted by one or more of halogen, C₁₋₆ alkyl and —OR²a; more preferably, the heterocyclyl is unsubstituted or substituted by one or two of halogen, methyl and methoxy; In further embodiments, L is —O—CH₂—, and R² is a 4- to 8-membered monocyclic heterocycle containing 1, 2 or 3 heteroatoms selected from oxygen, nitrogen, and sulfur as ring members, or a 6- to 12-membered bicyclic heterocycle containing 1, 2 or 3 heteroatoms selected from oxygen, nitrogen, and sulfur as ring members (preferably a bridged bicyclic ring), wherein the monocyclic or bicyclic heterocyclic group is unsubstituted or substituted by one or two of halogen, C₁₋₆ alkyl, —OR^{2a}, and —(CR^{2a}R^{2b})ₘ—OC(O)NR^{2c}R^{2d}, wherein each variable is as defined for formula (I); preferably, the heterocyclyl is unsubstituted or substituted by one or more of halogen, C₁₋₆ alkyl and —OR²a; more preferably, the heterocyclyl is unsubstituted or substituted by one or two of halogen, methyl and methoxy; in a further embodiments, L is —O—CH₂—, and R² is a monocyclic heterocyclic ring, which is an N-heterocyclobutanyl, pyrrolidyl or piperidyl, the ring is unsubstituted or substituted by one or two halogens or C₁₋₆ alkyl. In a further embodiment, L is —O—CH₂—, and R² is a bicyclic heterocycle, which is octahydropentadiene, wherein at least one carbon atom is substituted by a nitrogen atom, and one of the other carbon atoms is optionally substituted by an oxygen atom; Preferably R² is tetrahydro-1H-pyrrolizinyl (e.g. tetrahydro-1H-pyrrolidine-7-yl

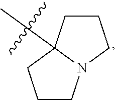 [SMILES: *C12CCCN1CCC2]

preferably (2R,7aS)-2-fluorotetrahydro-1H-pyrrolizin-7a (5H)-yl), tetrahydro-1H-furanopyrrole (e.g. tetrahydro-1H-furano[3,4-b] pyrrole-3a-yl

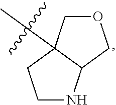 [SMILES: *C12CCNC1COC2]

tetrahydro-1H-furano[3pyrrole-3a-yl

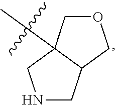 [SMILES: *C12CNCC1COC2]

tetrahydro-1H-furano[3pyrrole-6a-yl

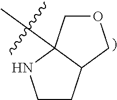 [SMILES: *C12COCC1CCN2]

and octahydrocyclopentapyrrolyl (e.g. octahyropentano[b]pyrrole-6a-yl

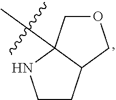 [SMILES: *C12COCC1CCN2]

octahydrocyclopentano[c]pyrrole-3a-yl

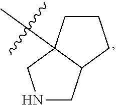 [SMILES: *C12CCCC1CNC2]

octahydrocyclopentano[b]pyrrole-3a-yl,

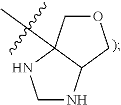 [SMILES: *C12COCC1NCN2]

and preferably R² is an azabicyclic [3.1.0] hexanyl, such as 3-azabicyclic[3.1.0] hexanyl(3-azabicyclic[3.1.0]hexan-1-yl), 2-azabicyclic[3.1.0]hexanyl(2-azabicyclic[3.1.0]hexan-1-yl), wherein the bicyclic heterocycle is unsubstituted or substituted by one or more of halogen, C₁₋₆ alkyl, —OR^{2a}, and —(CR^{2a}R^{2b})ₘ—OC(O)NR^{2c}R^{2d}, wherein the remaining variables are as defined for formula (I); preferably, the heterocyclic group is unsubstituted or substituted by one or more halogens, C₁₋₆ alkyl, and —OR^{2a}; more preferably, the heterocyclic group is unsubstituted or substituted by one or two of halogen, methyl and methoxy groups.

In a further preferred embodiment, L-R² is

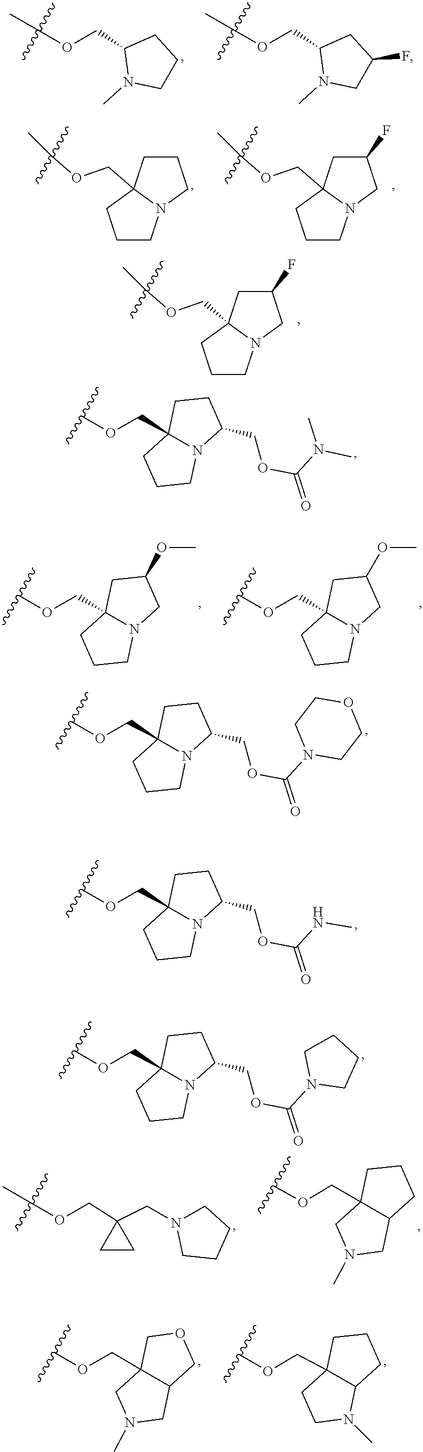

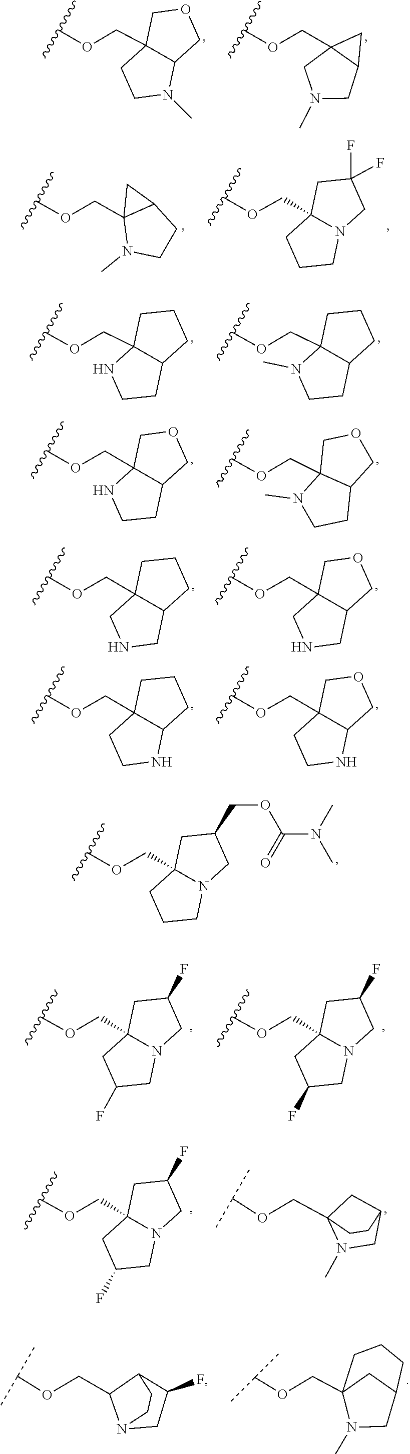

In some embodiments, R³ in compound of formula (I) and (II) is
a. monocyclic aryl or heteroaryl, selected from phenyl, pyridyl, pyrimidinyl and pyrazinyl;
b. bicyclic aryl or heteroaryl, selected from naphthyl group (e.g., naphthalene-1-yl, naphthalene-2-yl, naphthalene-3-yl, naphthalene-4-yl, naphthalene-5-yl, naphthalene-6-yl, naphthalene-7-yl, naphthalene-8-yl), isoquinolinyl (e.g. isoquinoline-1-yl, isoquinoline-3-yl, isoquinoline-4-yl, isoquinoline-5-yl, isoquinoline-6-yl, isoquinoline-7-yl and isoquinoline-8-yl), tetrahydroisoquinoline (e.g., 5,6,7,8-tetrahydroisoquinoline-1-yl, 5,6,7,8-tetrahydroisoquinoline-3-yl, and 5,6,7,8-tetrahydroisoquinoline-4-yl), tetrahydrogenated naphthalene (e.g. 5,6,7,8-tetrahydronaphthalen-1-yl, 5,6,7,8-tetrahydronaphthalen-2-yl, 5,6,7,8-tetrahydronaphthalen-3-yl, 5,6,7,8-tetrahydronaphthalen-4-yl);
c. tricyclic aryl or heteroaryl, selected from

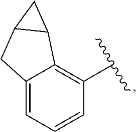 [SMILES: *c1cccc2c1C1CC1C2]

the aryl or heteroaryl is substituted by 1 to 3 R⁸, R⁸ is each independently selected from: halogen, C₁₋₆ alkyl optionally substituted by halogen, C₂-C₆ alkynyl, —P(═O)R^{8a}R^{8b}, —NR^{8a}R^{8b}, —OR^{8a}, —SR^{8a}, C₃-C₆ cycloalkyl optionally substituted by halogen or C₁₋₆ alkyl, wherein R^{8a} and R^{8b} are each independently of hydrogen and C₁₋₆ alkyl.

In some embodiments, R³ in compound of formula (I) and (II) is a naphthalene-1-yl (wherein R³ is the naphthyl, and the position connected to the parent structure is 1), and the 3 position of the naphthalene-1-yl is unsubstituted or substituted by—OR^{8a}, —P(═O)R^{8a}R^{8b} or —NR^{8a}R^{8b}, wherein R^{8a} and R^{8b}are each independently hydrogen and C₁₋₆ alkyl, preferably 3 position of the naphthalene-1-yl is unsubstituted or substituted by—OH or —NH₂, and one or two of the positions 5, 6, 7 and 8 of the naphthalene-1-yl are substituted, and the substituents are independently selected from halogen, C₁₋₆ alkyl optionally substituted by halogen, C₂-C₆ alkynyl, —P(═O)R^{8a}R^{8b}, —NR^{8a}R^{8b}, —OR^{8a}, —SR^{8a}, and C₃-C₆ cycloalkyl optionally substituted by halogen or C₁₋₆ alkyl, wherein R^{8a} and R^{8b} are each independently hydrogen and C₁₋₆ alkyl. In some preferred embodiments, R³ in compound of formula (I) and (II) is naphthalen-1-yl (wherein R³ is the naphthyl, and the position connected to the parent structure is 1), the 3 position of the naphthalen-1-yl is unsubstituted or substituted by —OH or —NH₂, and at least one of the 7 and 8 position of the naphthalen-1-yl is substituted, the substituents are independently selected from halogen, C₁₋₆ alkyl optionally substituted by halogen, C₂-C₆ alkynyl, —P(═O)R^{8a}R^{8b}, —NR^{8a}R^{8b}, —OR^{8a}, —SR^{8a}, and C₃-C₆ cycloalkyl optionally substituted by halogen or C₁₋₆ alkyl, wherein R^{8a} and R^{8b} are each independently hydrogen and C₁₋₆ alkyl. In some preferred embodiments, R³ in compounds of formulas (I) and (II) is naphthalene-1-yl (wherein R³ is the naphthyl, and the position connected to the parent structure is 1), the 3 position of the naphthalen-1-yl is unsubstituted or substituted by —OH or —NH₂, and at least one of the 7 and 8 position of the naphthalen-1-yl is substituted (e.g. both position 7 and 8 of the naphthyl are unsubstituted, 7 or 8 position of the naphthalen-1-yl is substituted), the substituents are independently selected from halogen, C₁₋₆ alkyl optionally substituted by halogen, C₂-C₆ alkynyl, —P(═O)R^{8a}R^{8b}, —NR^{8a}R^{8b}, —OR^{8a}, —SR^{8a}, C₃-C₆ cycloalkyl optionally substituted by halogen or C₁₋₆ alkyl, wherein R^{8a} and R^{8b} are each independently hydrogen and C₁₋₆ alkyl; Preferably, the substituents are selected independently from F, Cl, methyl, ethyl, isopropyl, trifluoromethyl, ethynyl, —P(═O)(CH₃)₂ and cyclopropyl.

In some embodiments, R³ in compound of formula (I) and (II) is

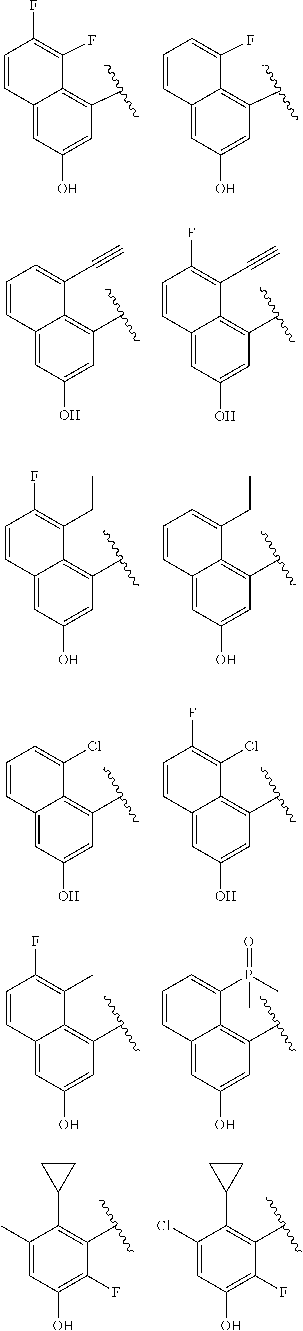

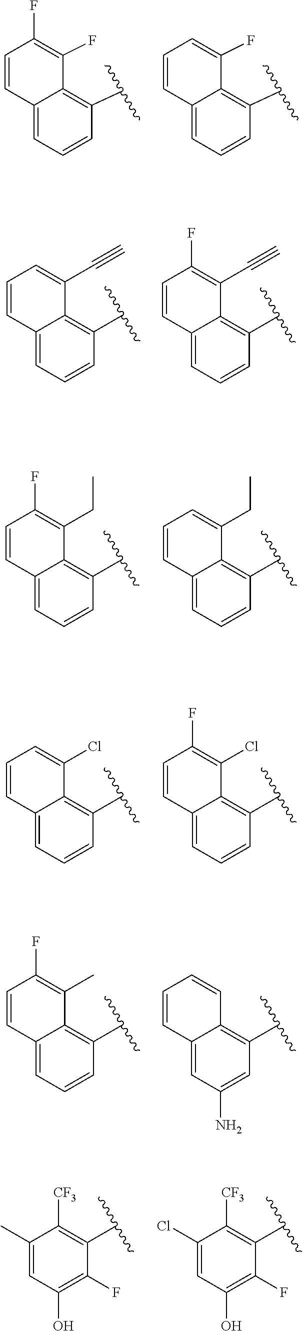

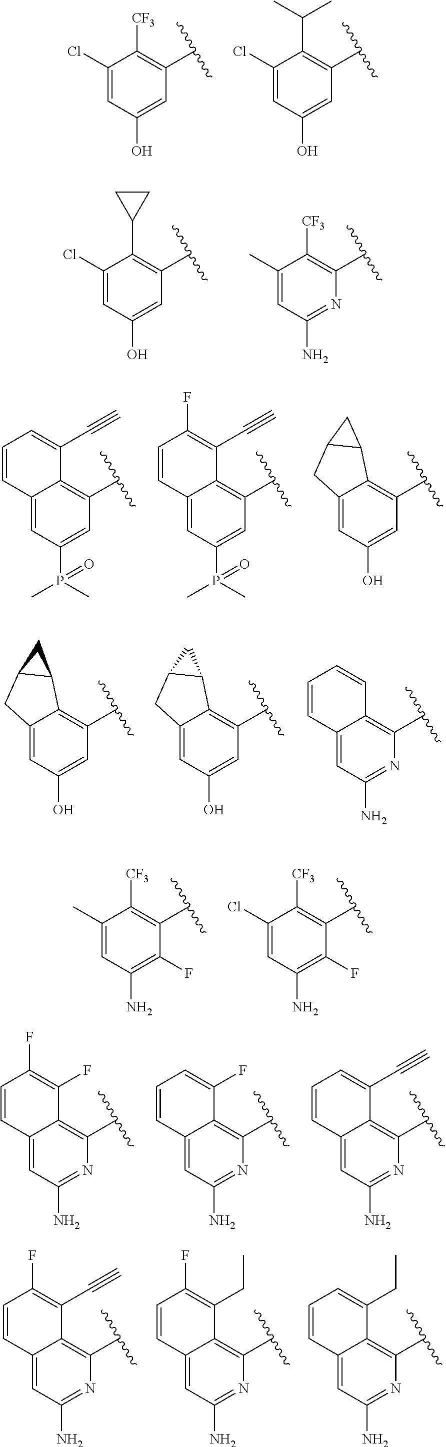

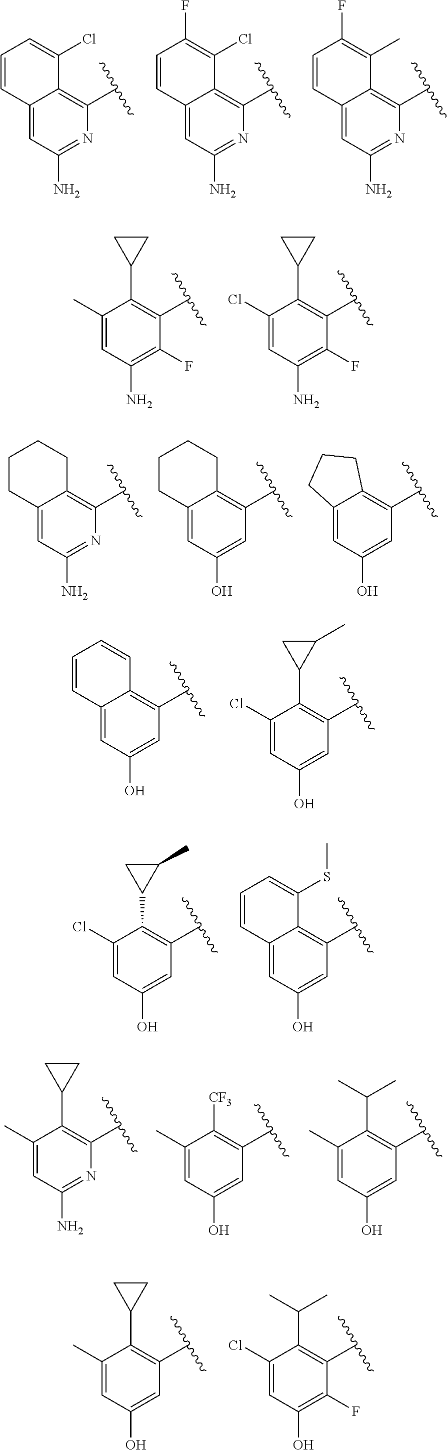

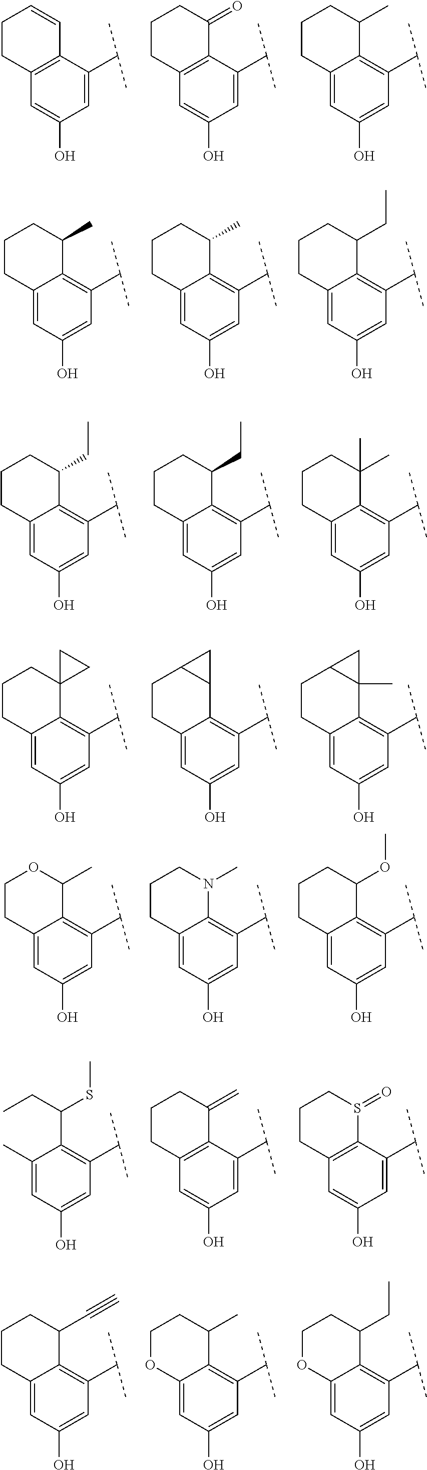

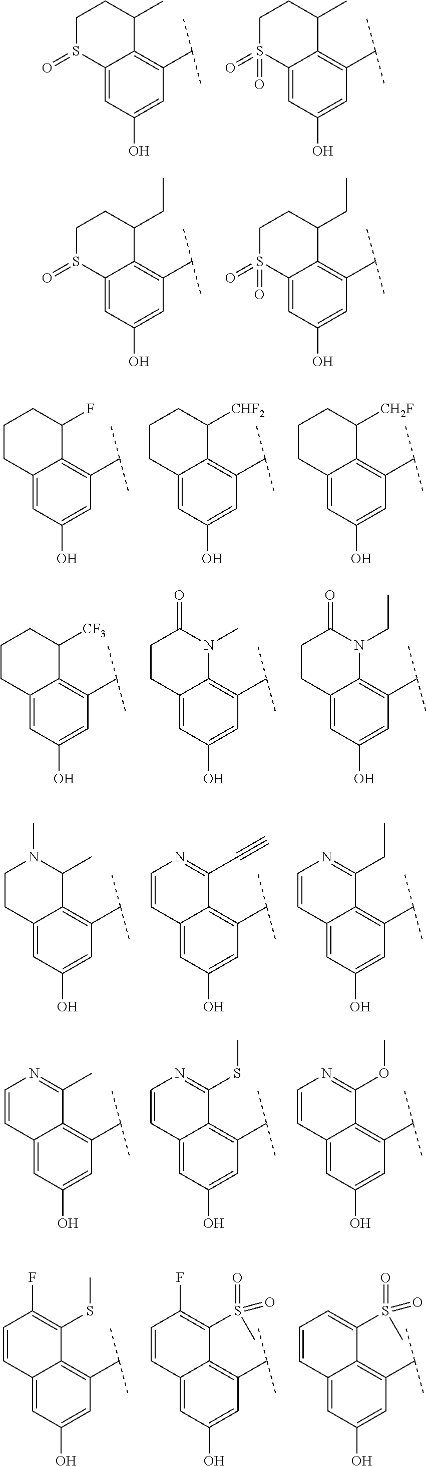

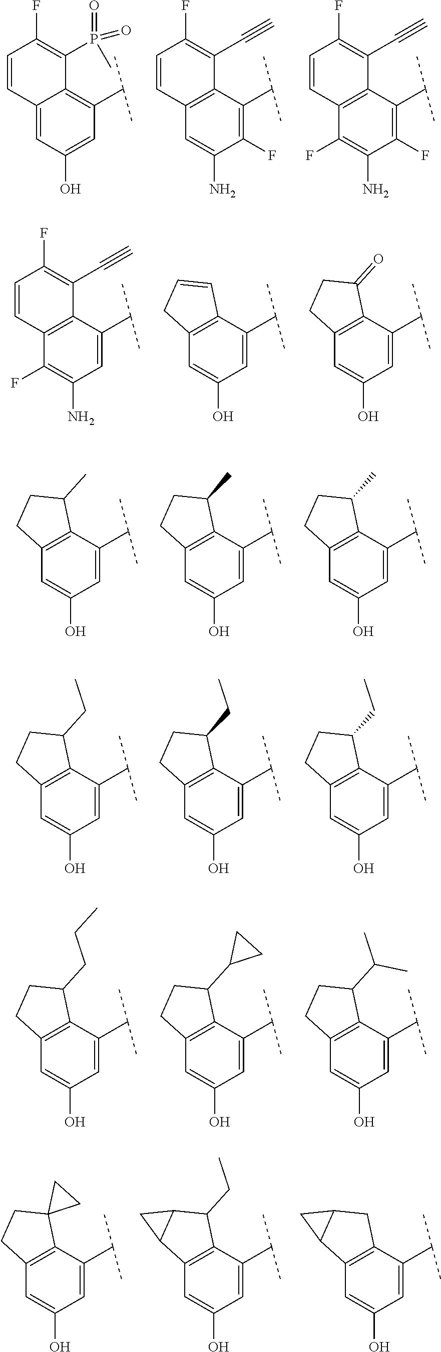

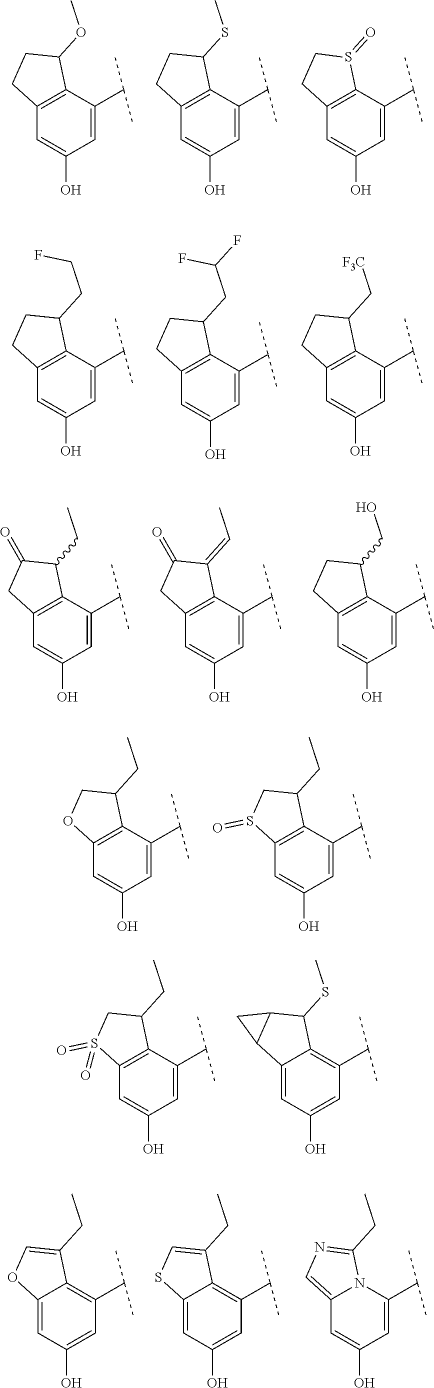

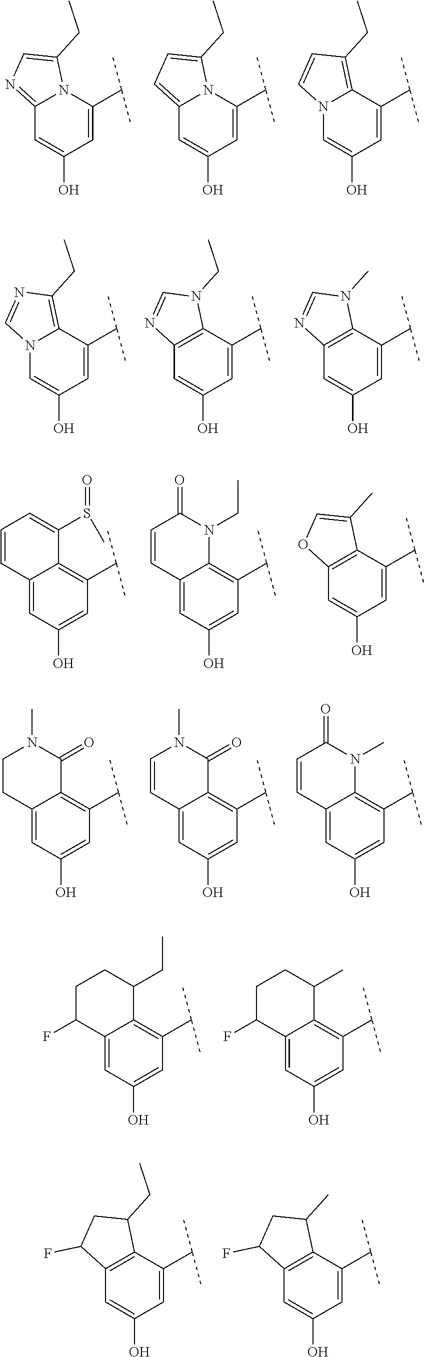

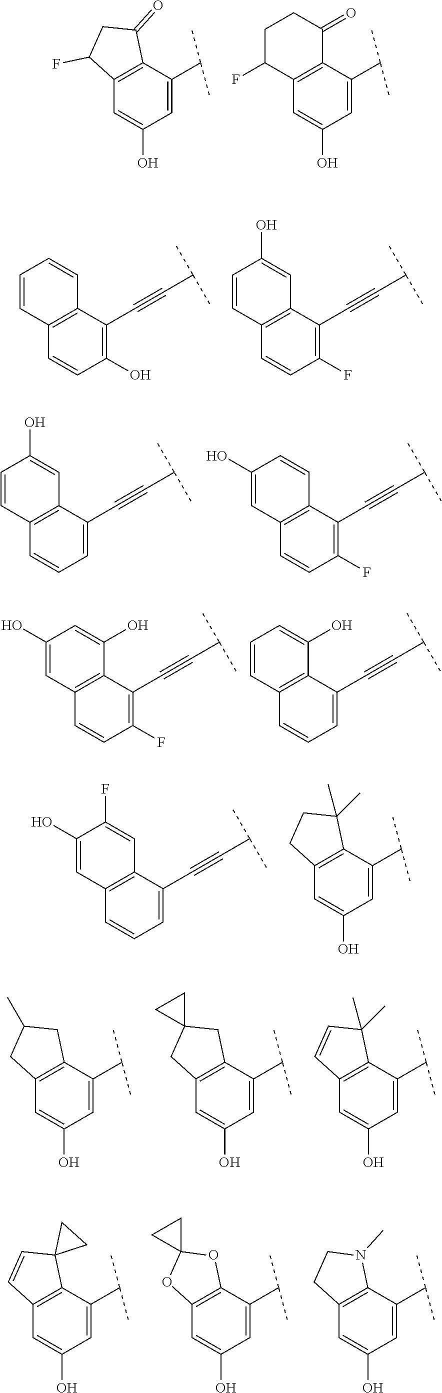

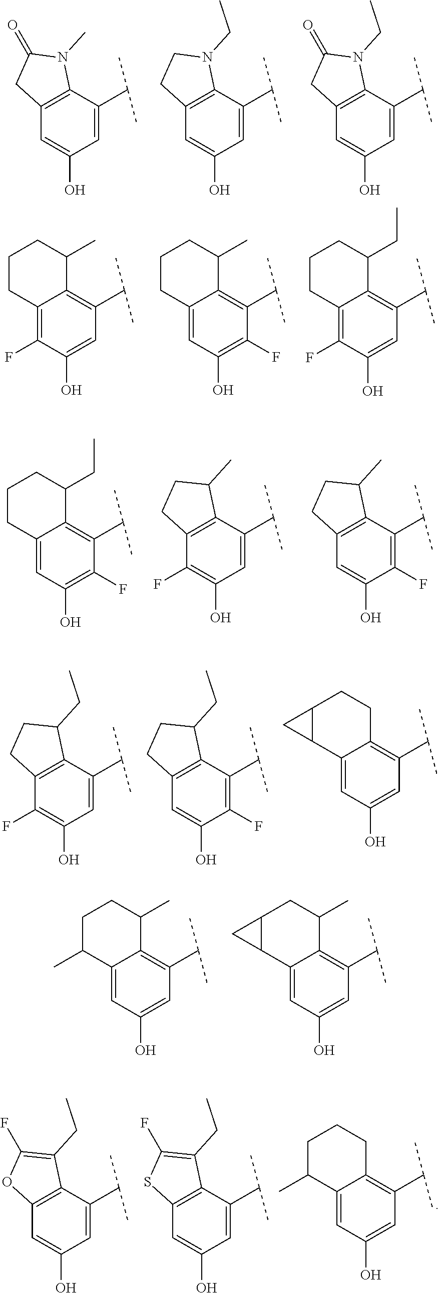

In some embodiments, the compound of formula (I) is

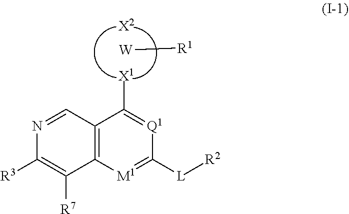

wherein:

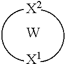

is

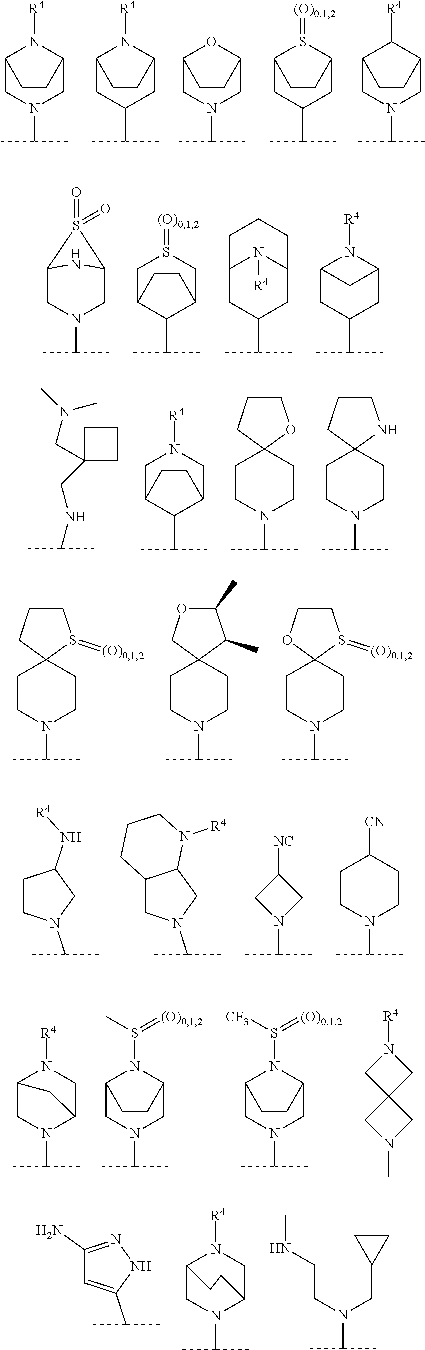

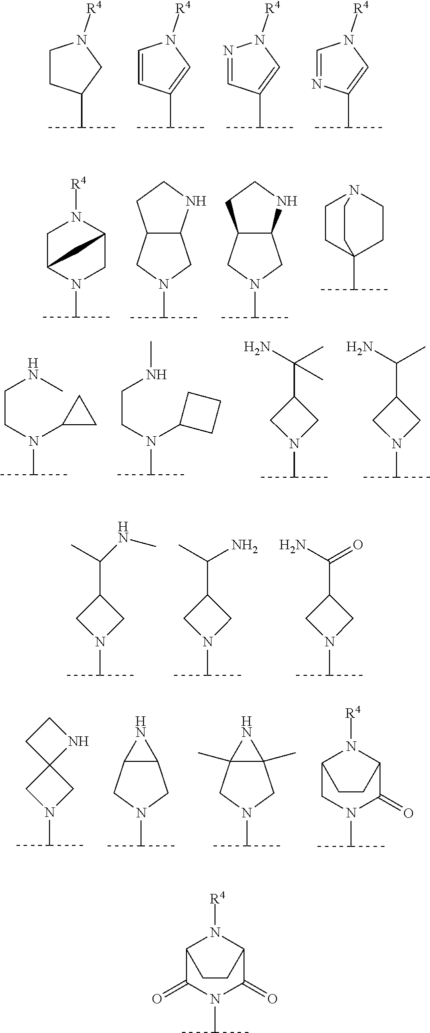

and the remaining variables are as defined for formula (I) or (II).

In some embodiments, the compound of formula (I) is as represented by formula (I-1-1):

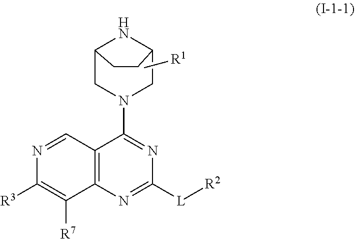

wherein:
L-R² is

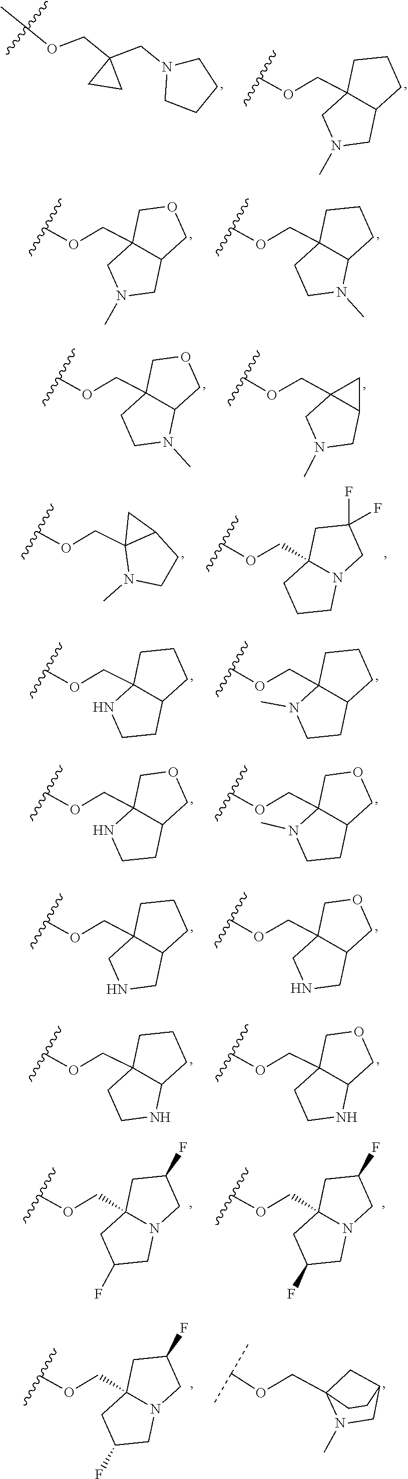

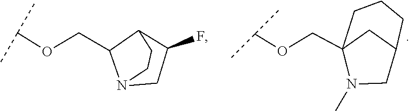

and the remaining variables are as defined for formula (I) or (II).

In some embodiments, the compound of formula (I) is as represented by formula (I-1-1):

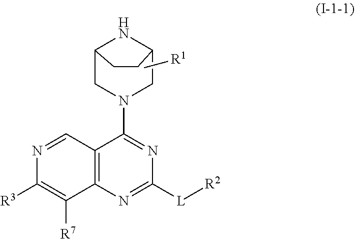

wherein:
L-R² is

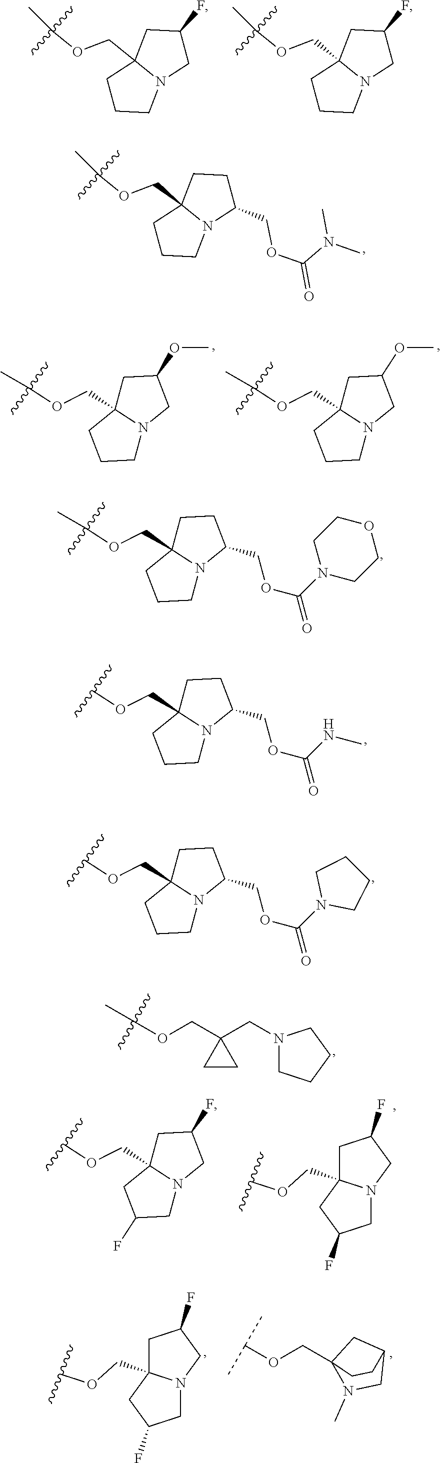

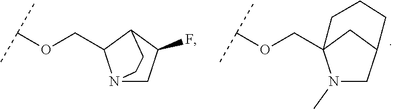

R³ is

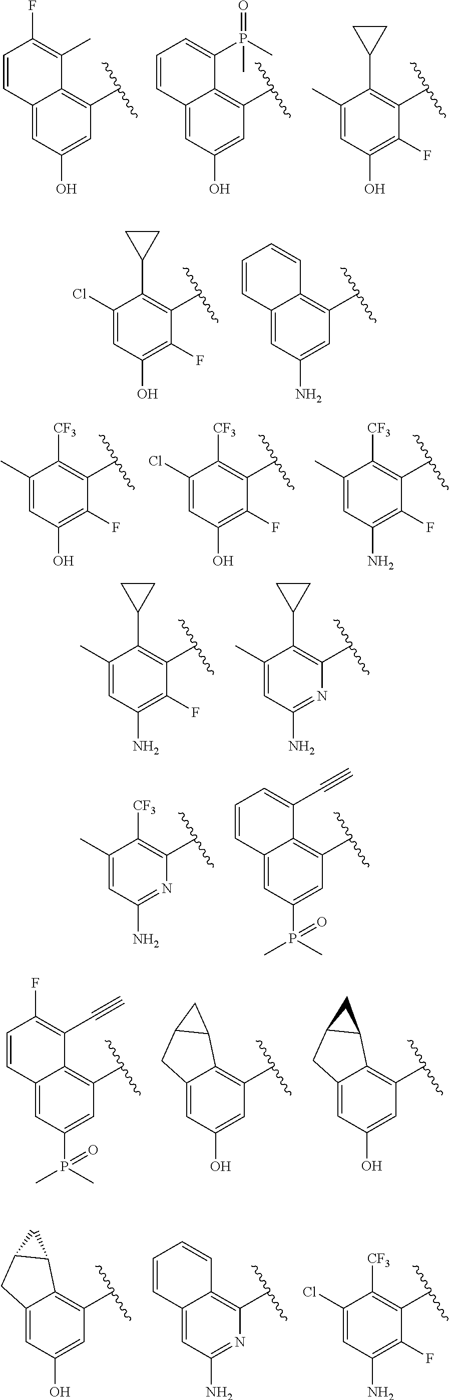

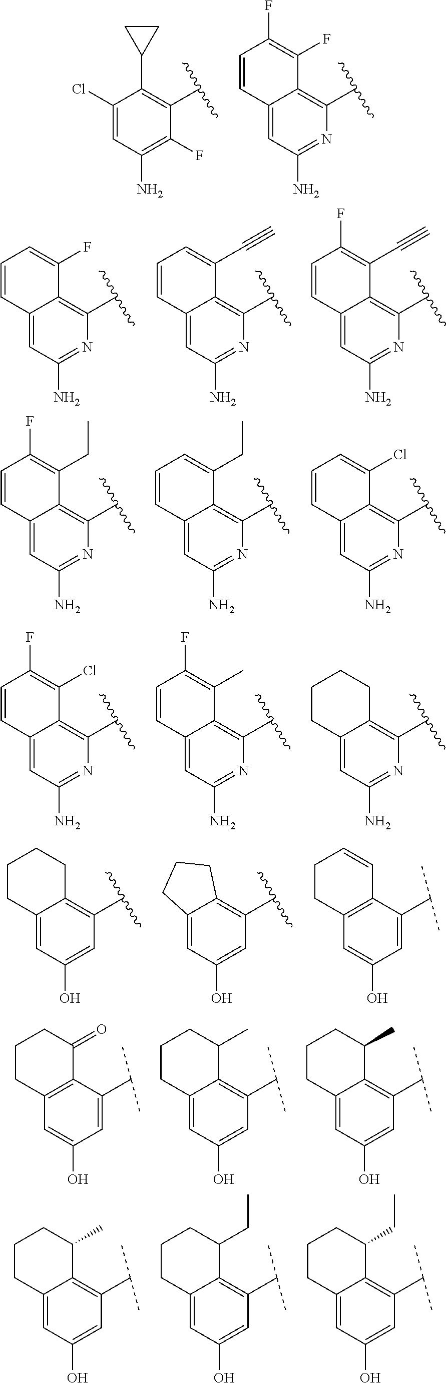

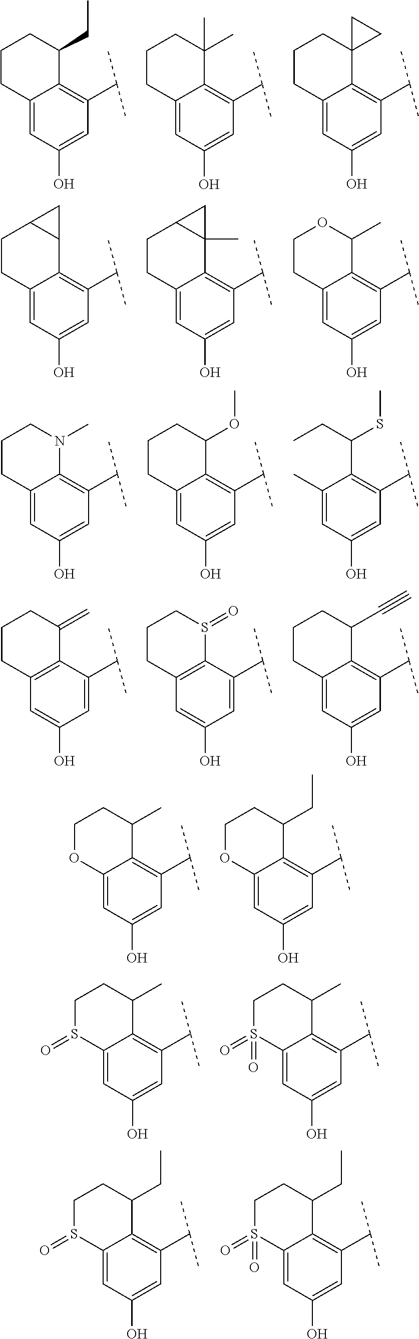

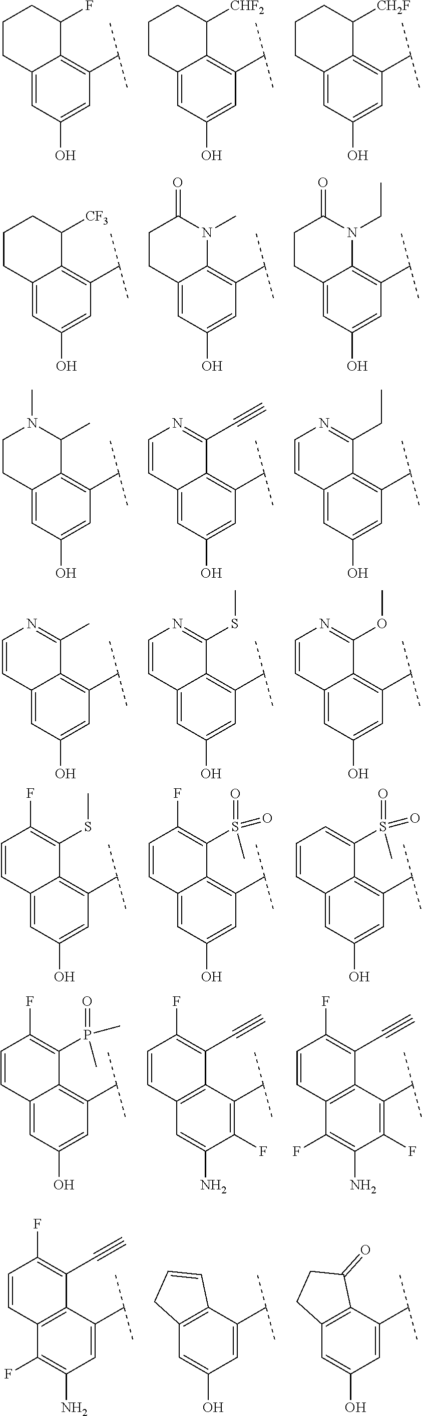

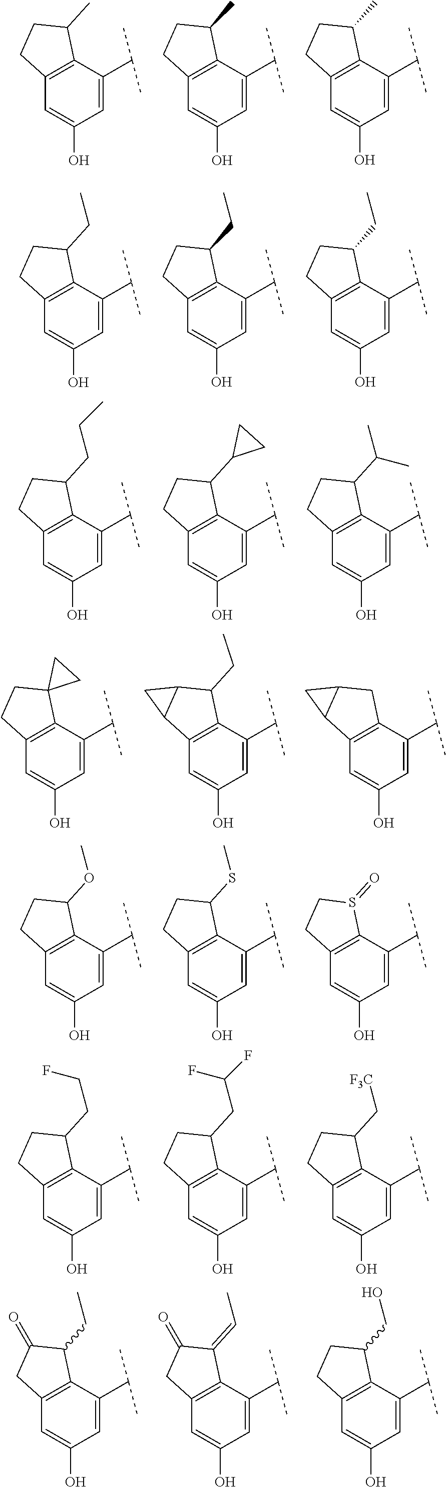

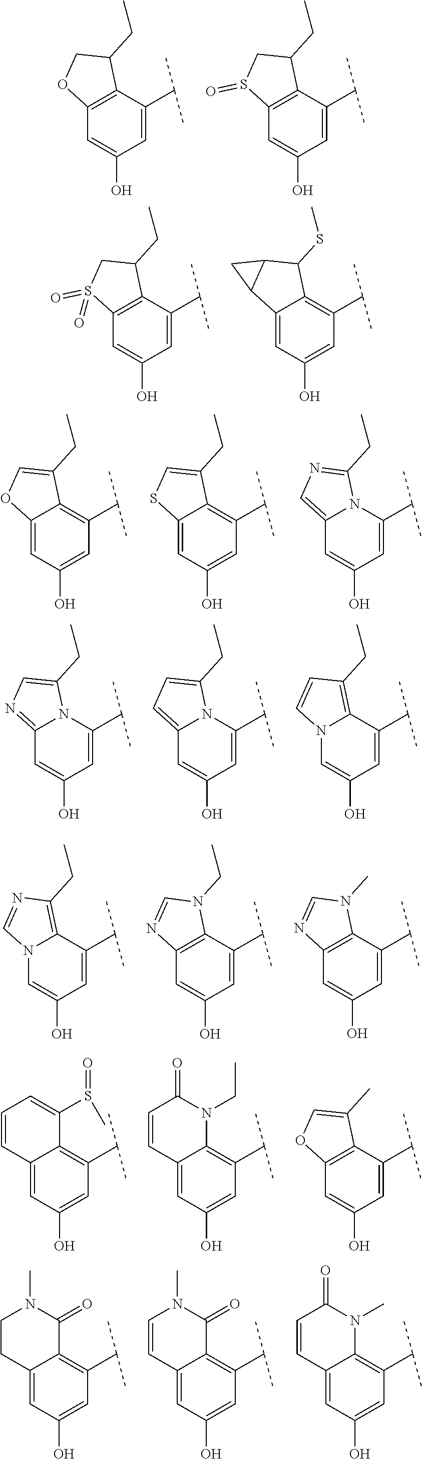

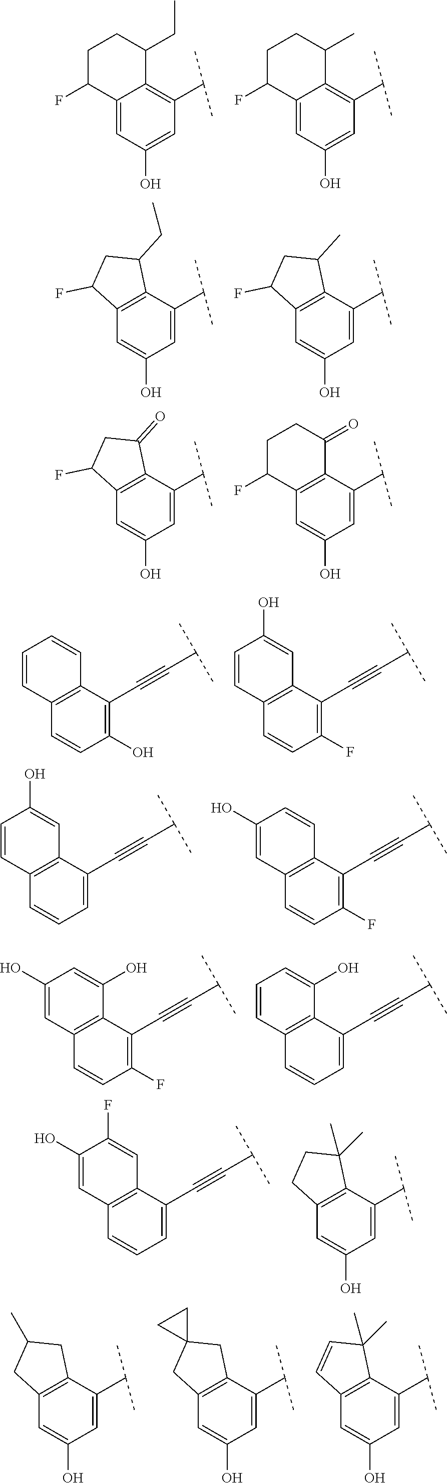

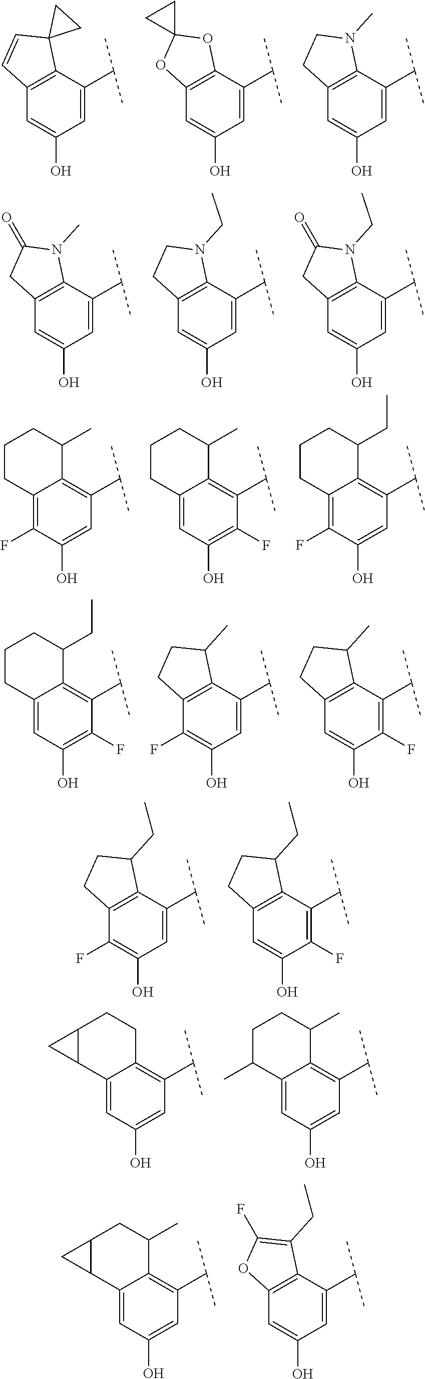

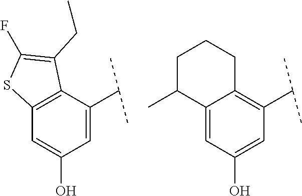

R⁷ is hydrogen, fluorine, chlorine, trifluoroethoxy, cyclopropyloxy or hydroxyl, and the remaining variables are as defined for formula (I) or (II).

In some embodiments, the compound of formula (I) is as represented by formula (I-2), (I-3), (I-4), (I-5) and (I-6):

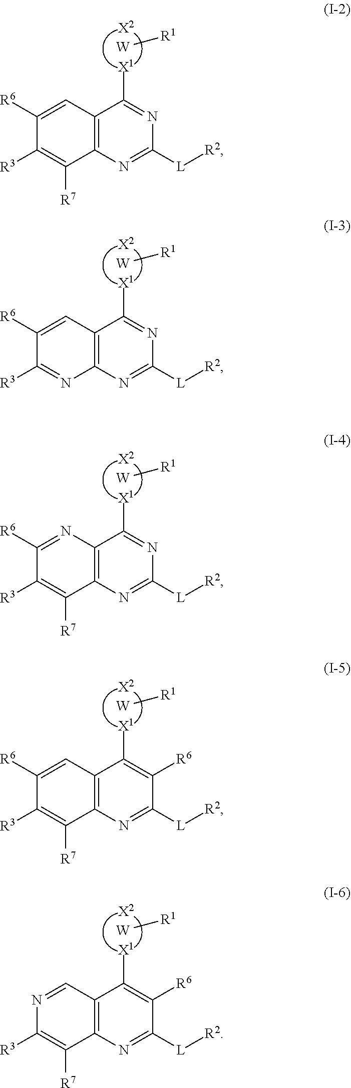

wherein
L-R² is

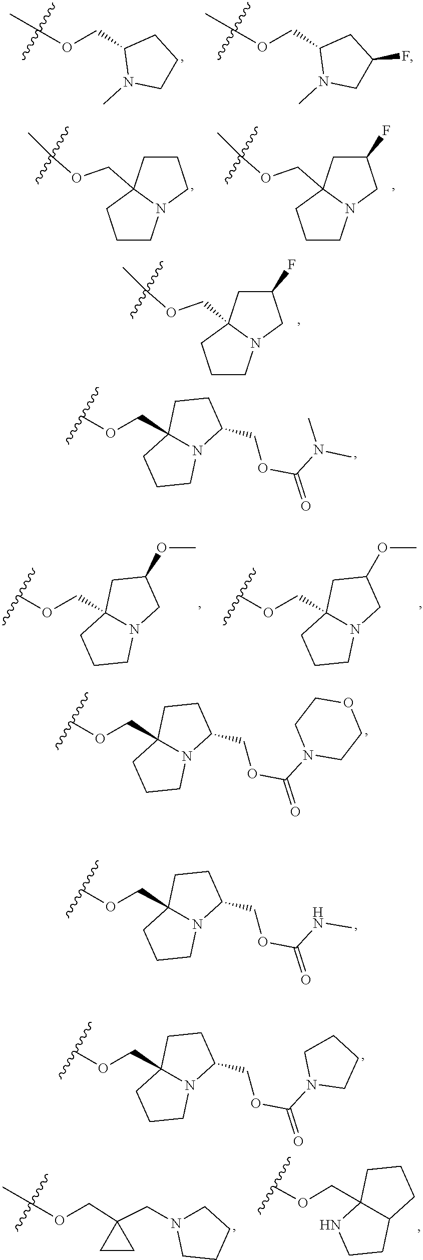

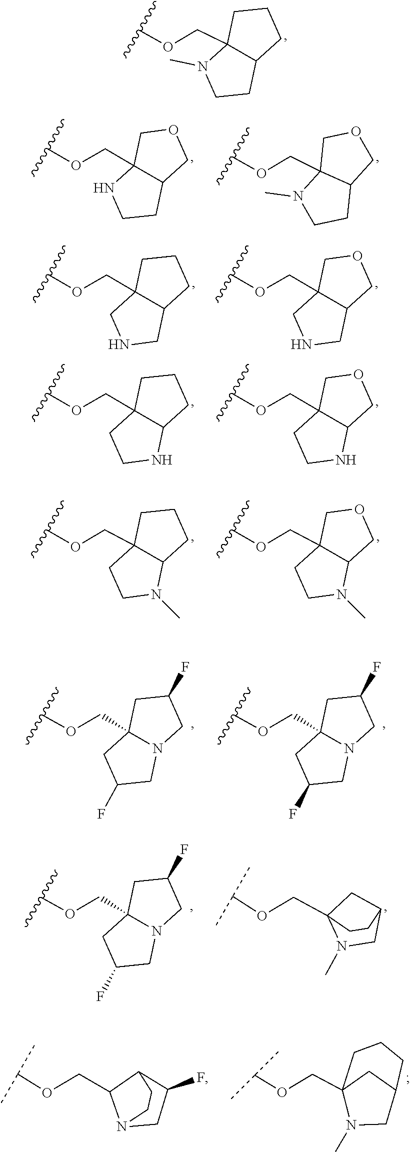

R³ is

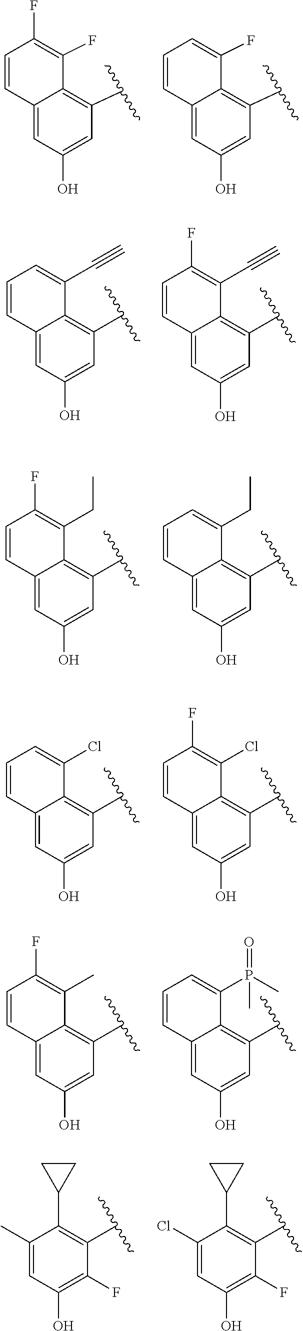

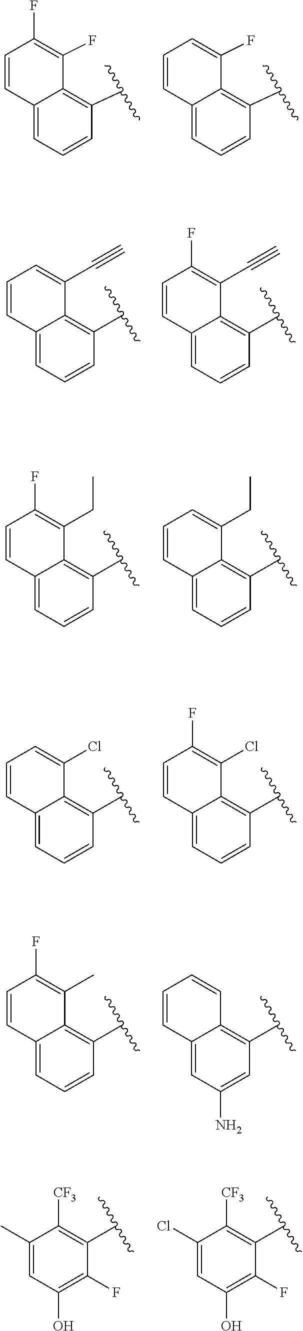

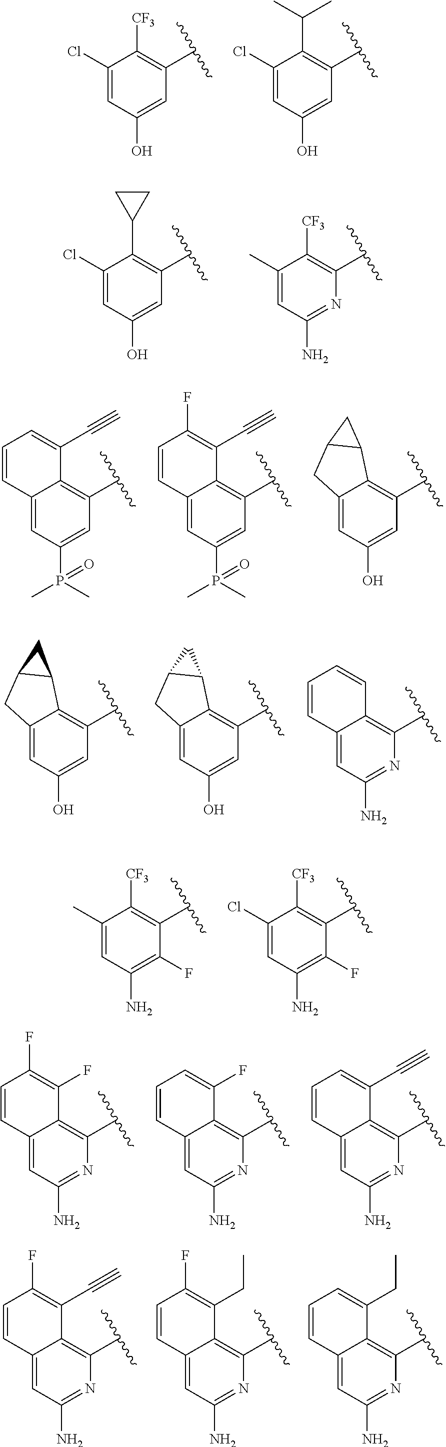

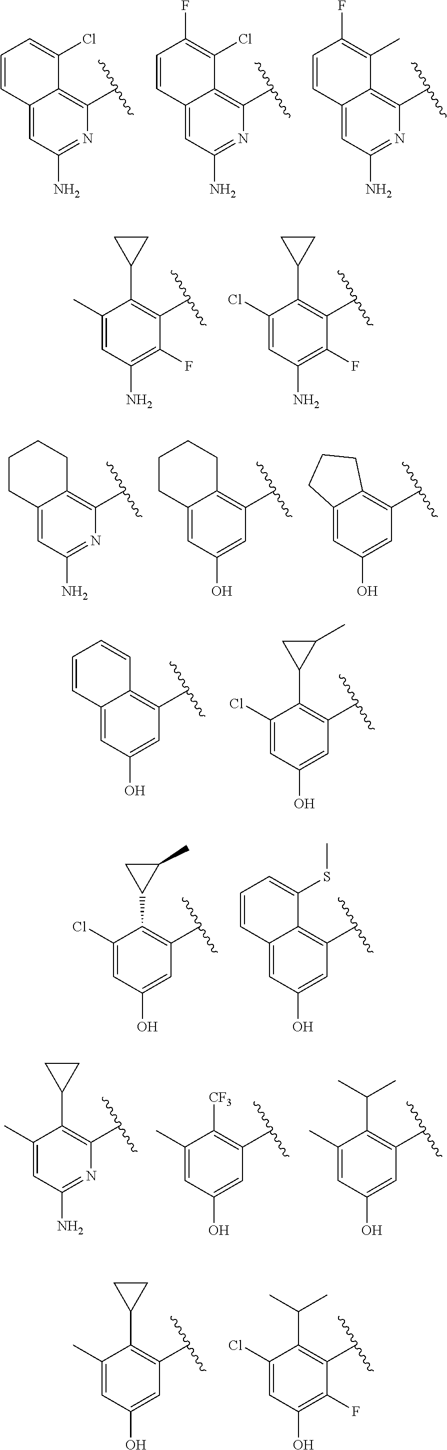

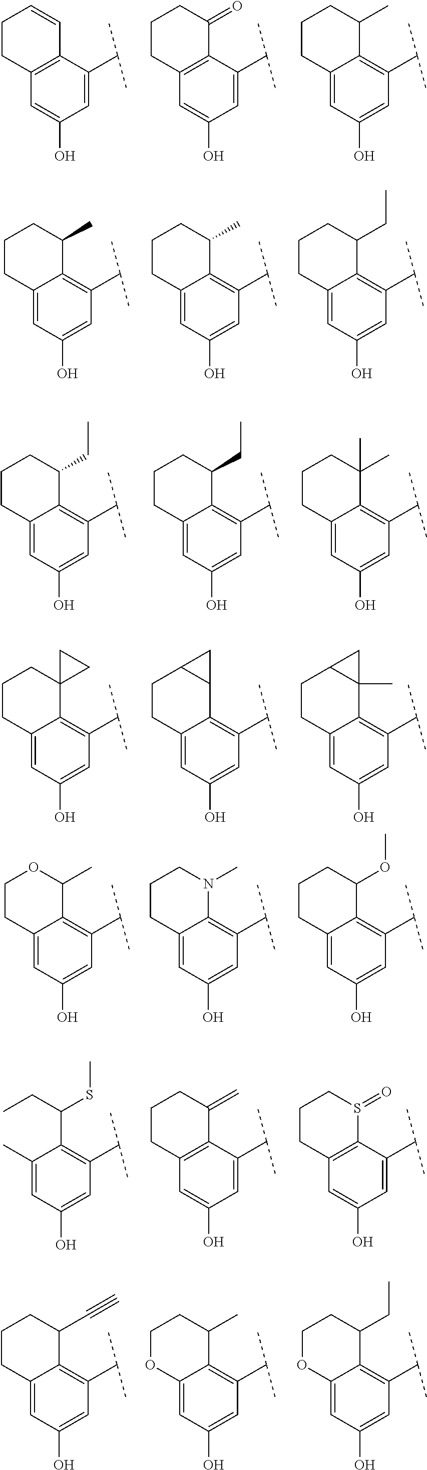

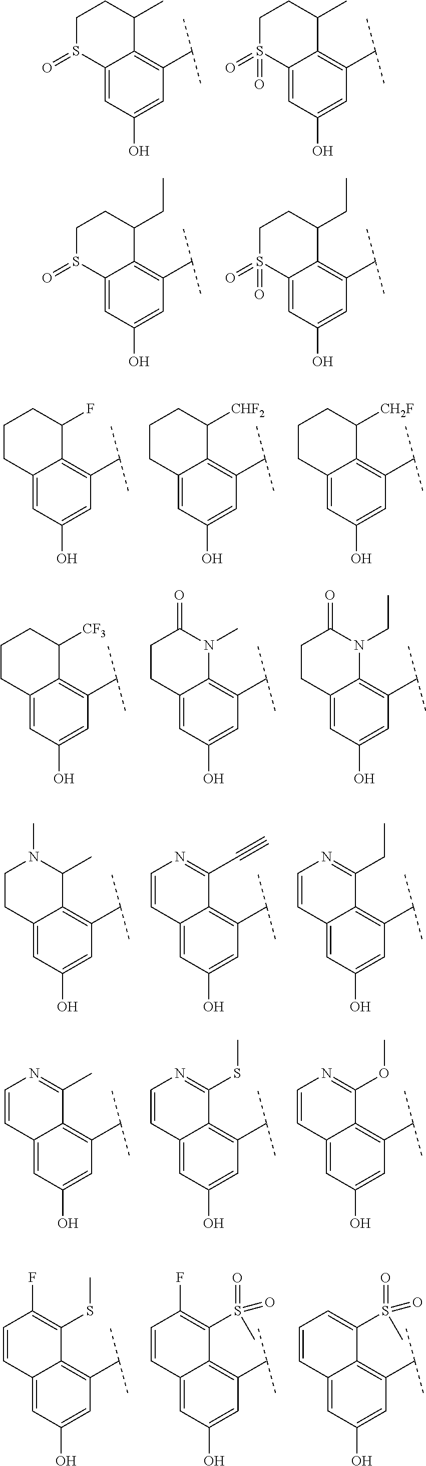

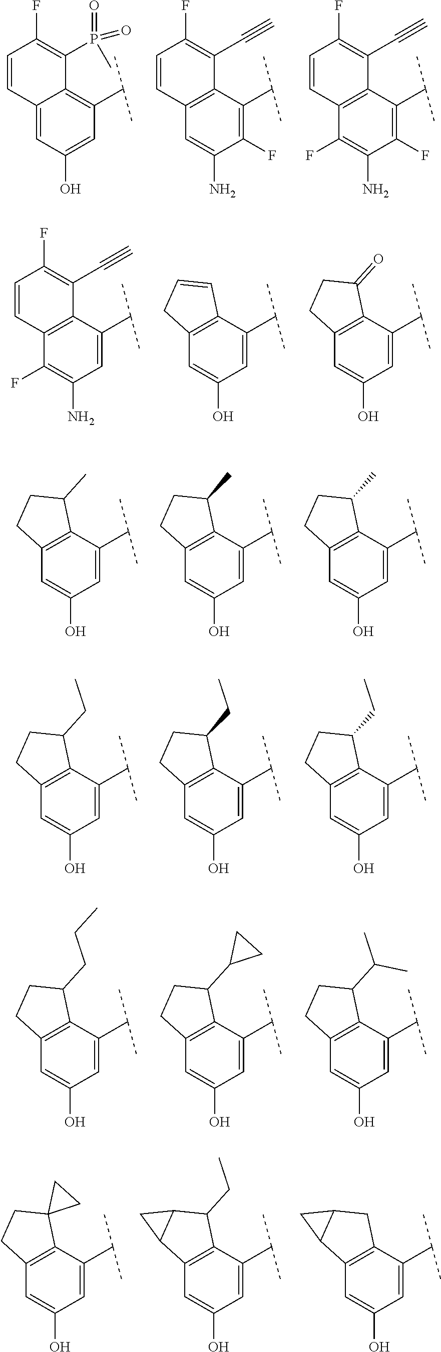

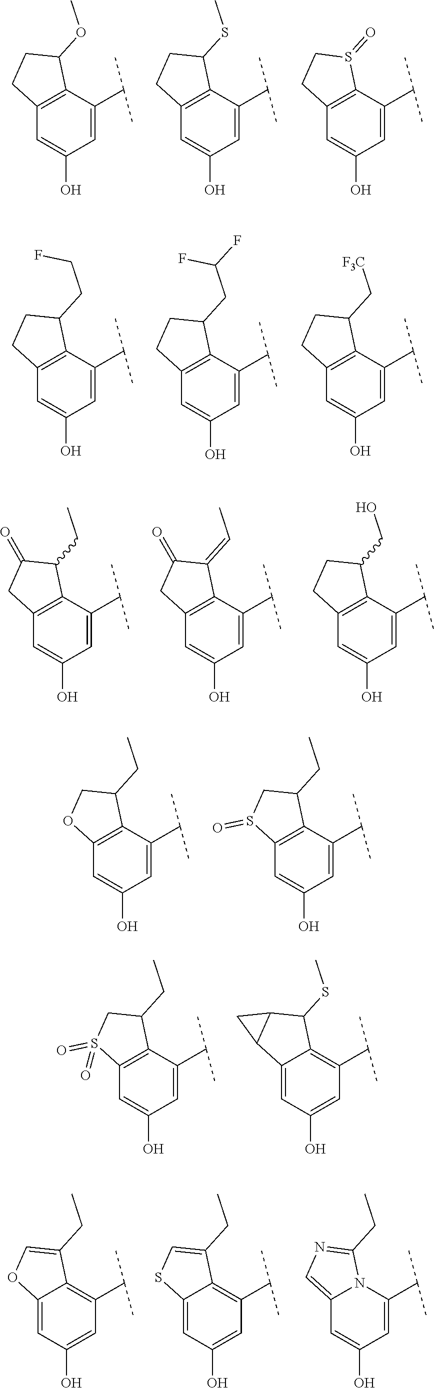

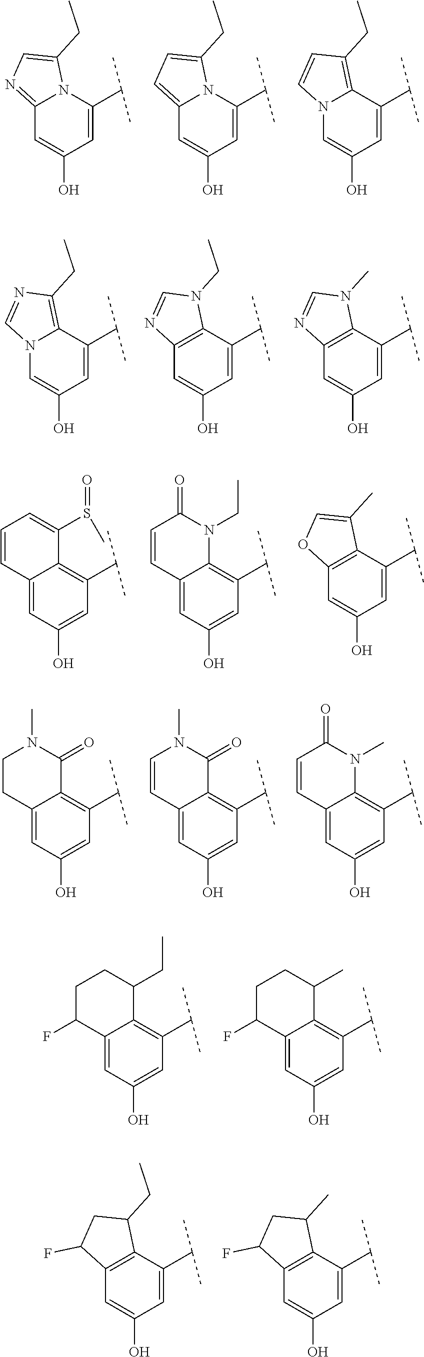

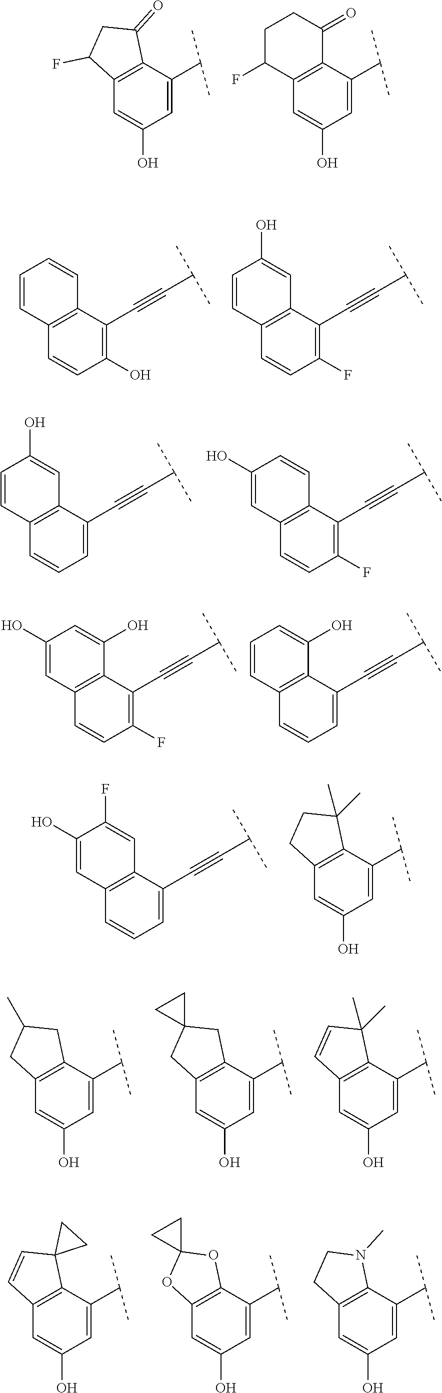

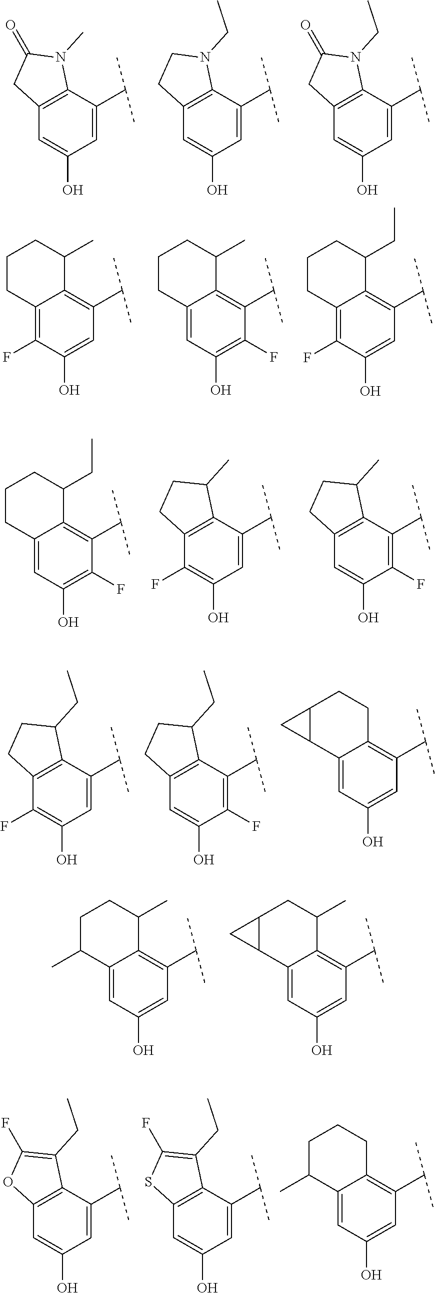

and the remaining variables are as defined for formula (I) or (II).

In some embodiments, the compound of formula (I) is

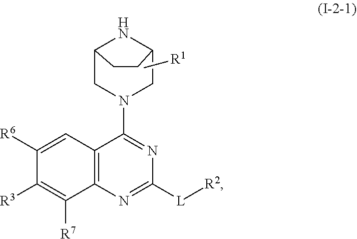

wherein
R³ is

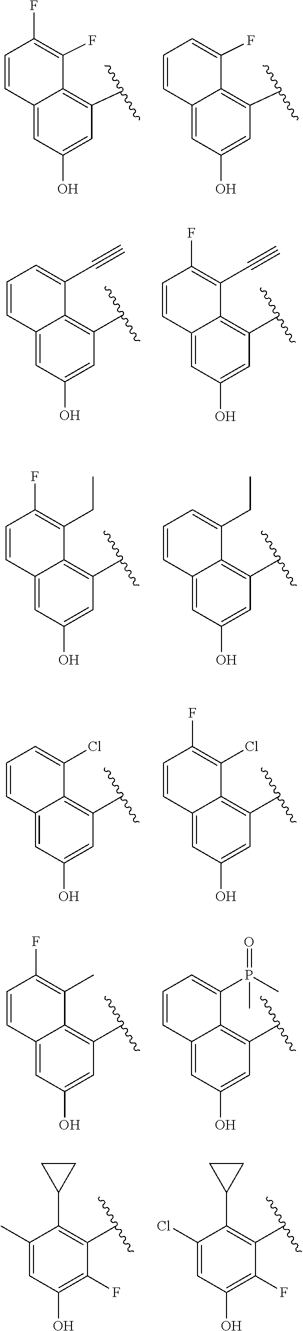

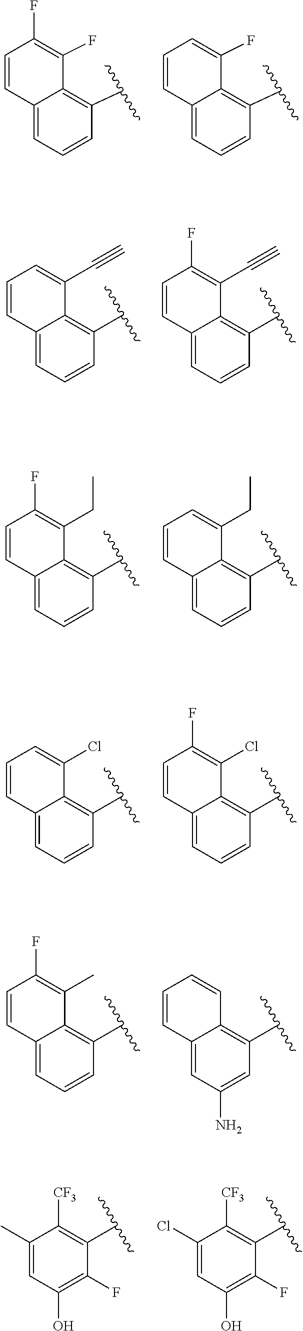

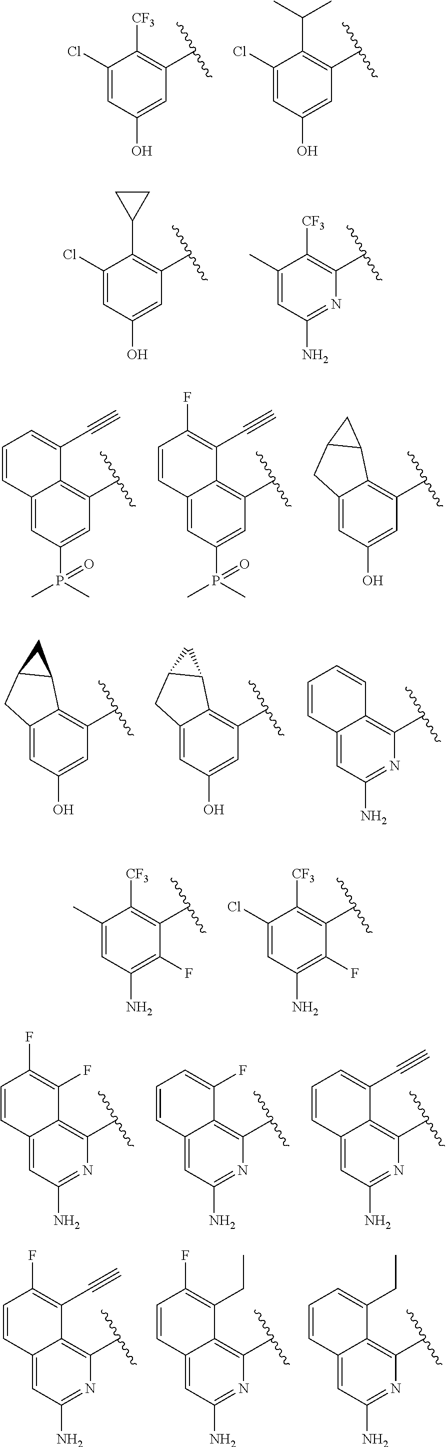

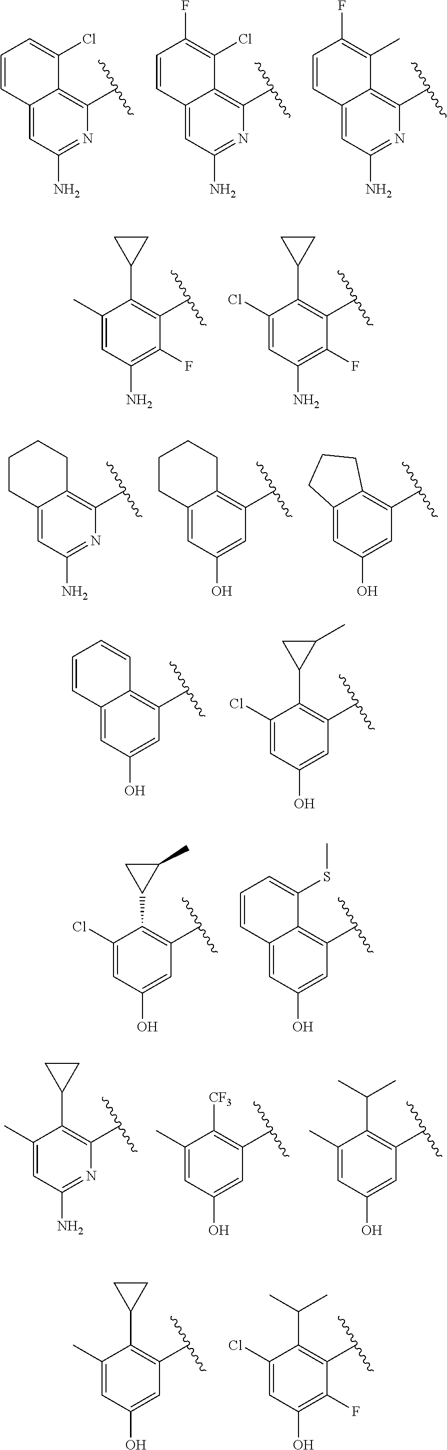

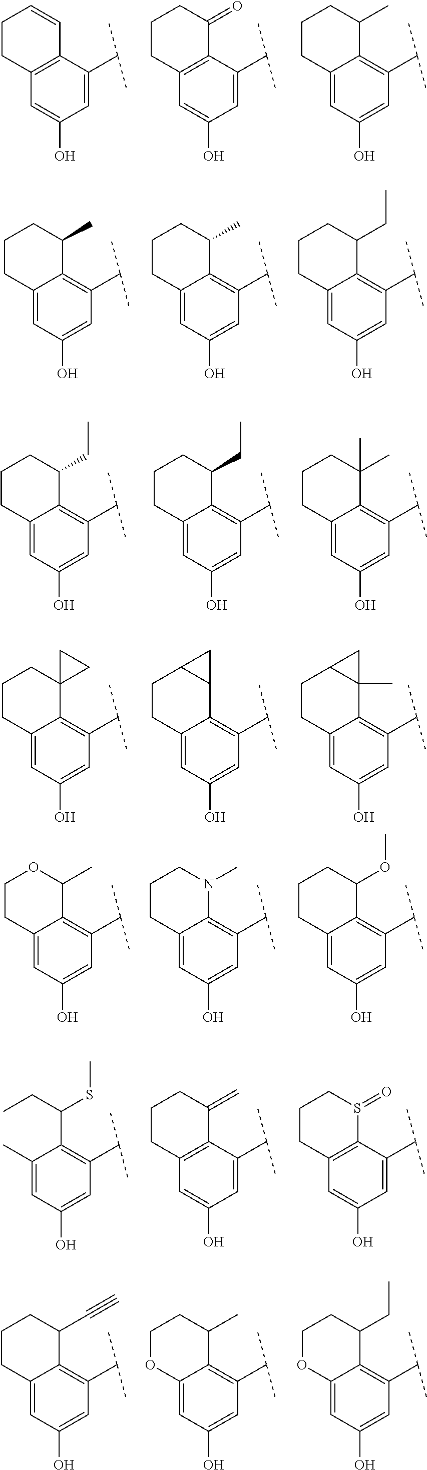

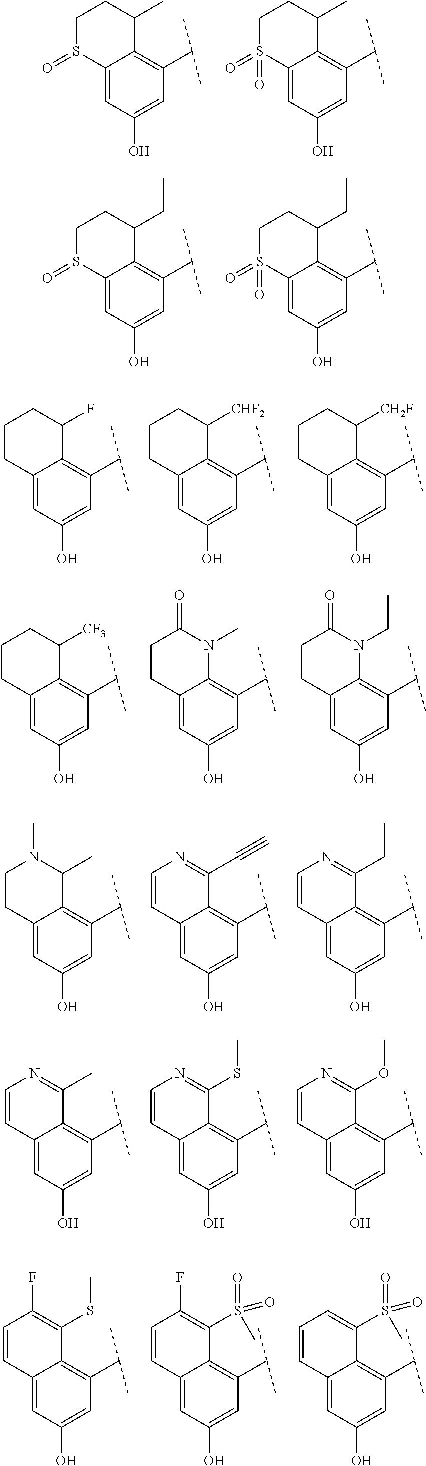

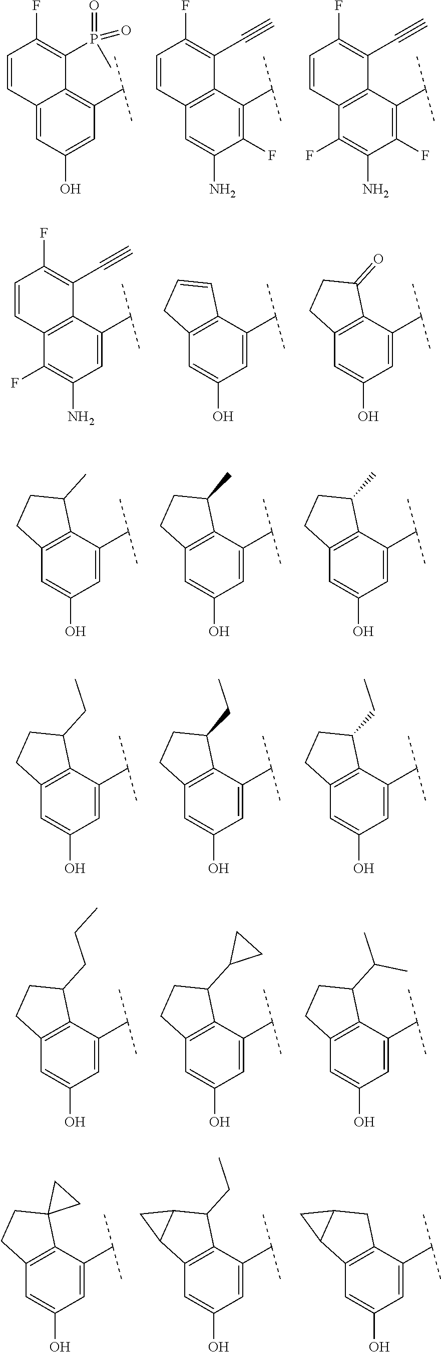

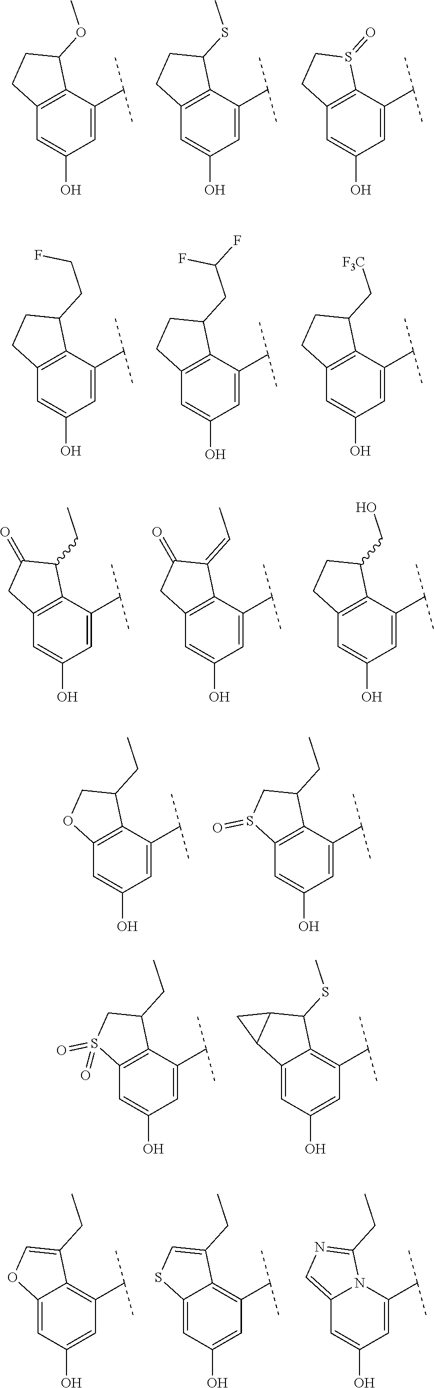

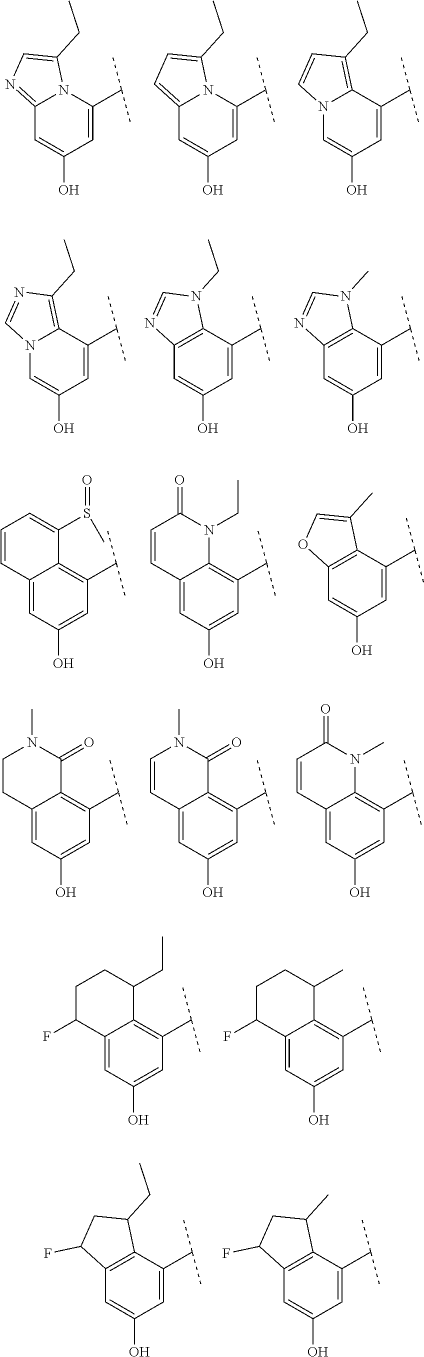

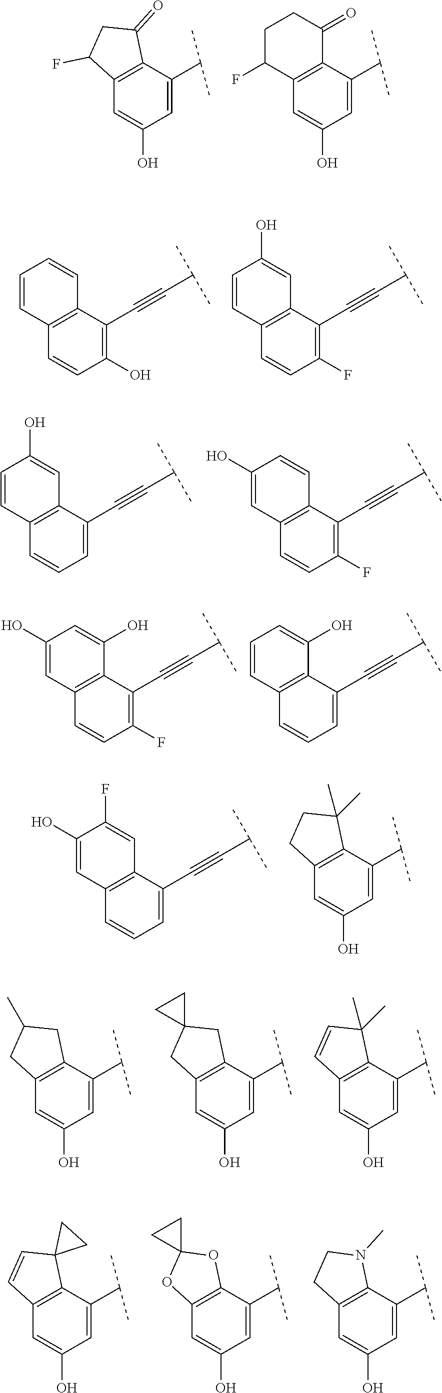

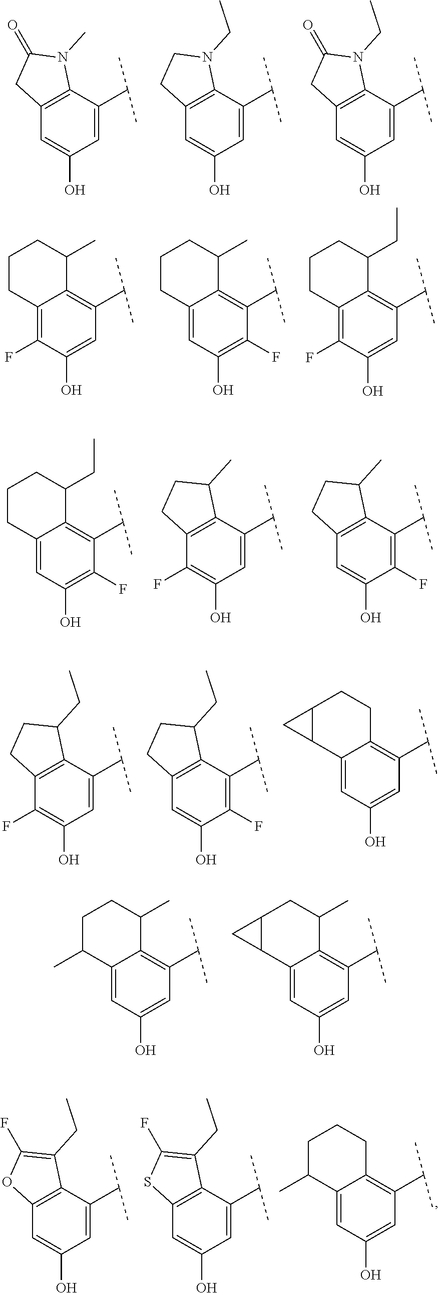

and
L-R² is

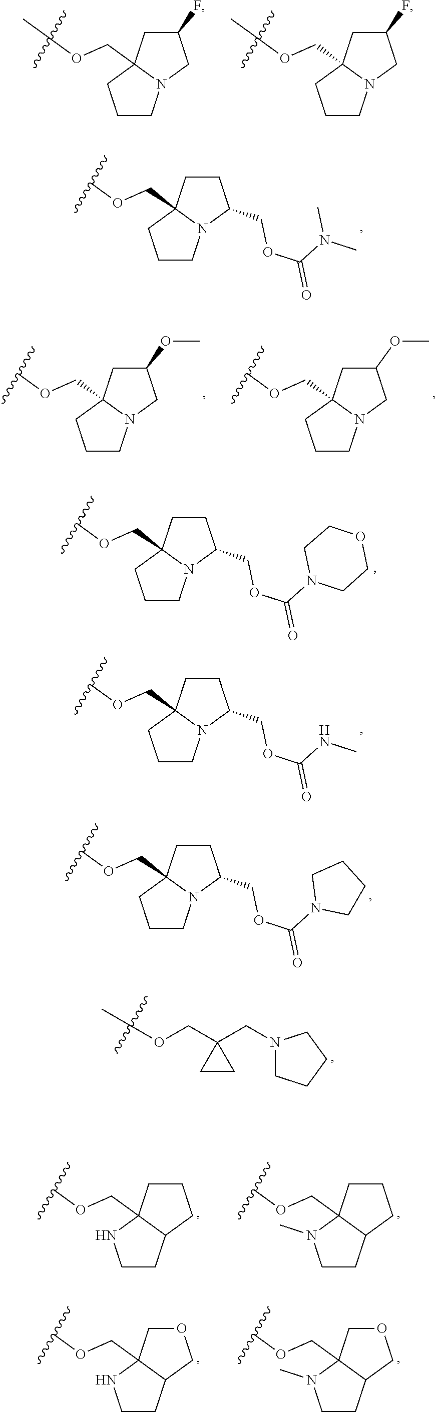

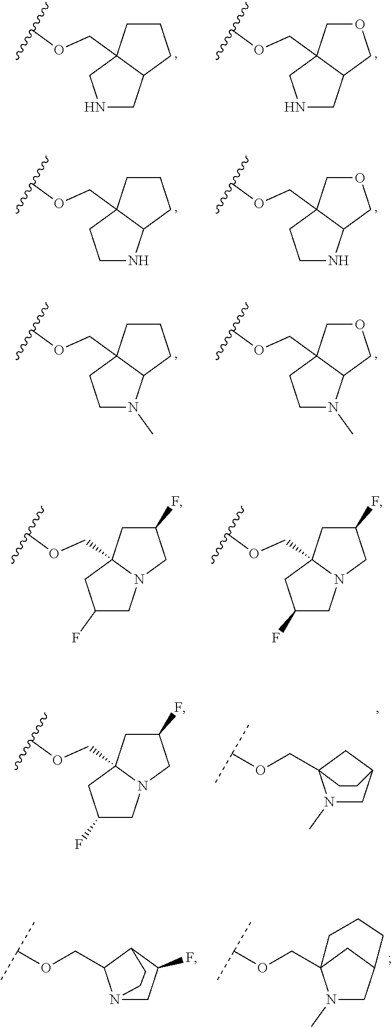

more preferably

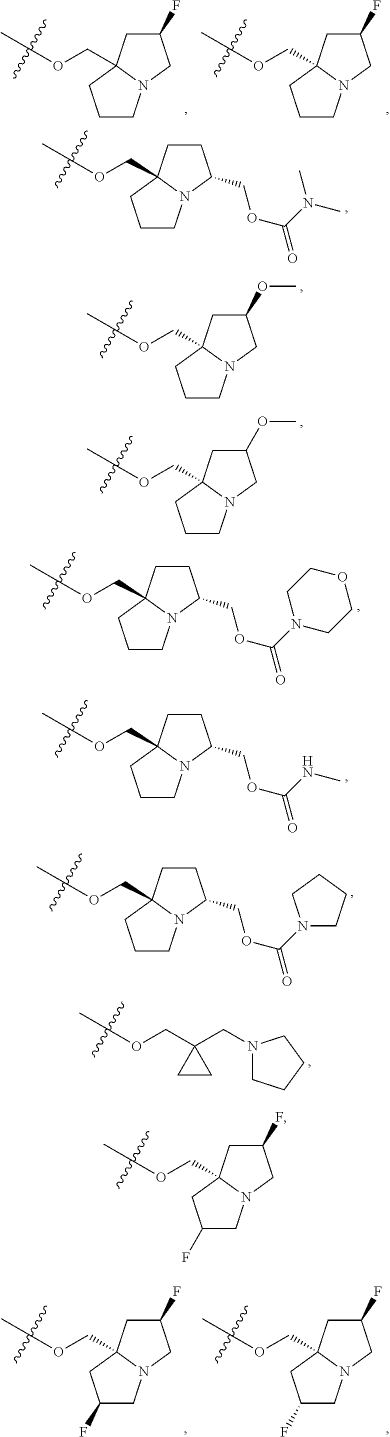

R⁶ and R⁷ are each independently hydrogen, alkyl ethynyl, cyano, cyclopropoxy, trifluoroethoxy, hydroxyl, fluorine, or halogen, and the remaining variables are as defined for formula (I) or (II).

In some embodiments, the compound of formula (I) is as represented by formula (I-1-2), (I-1-3) and (I-1-4):

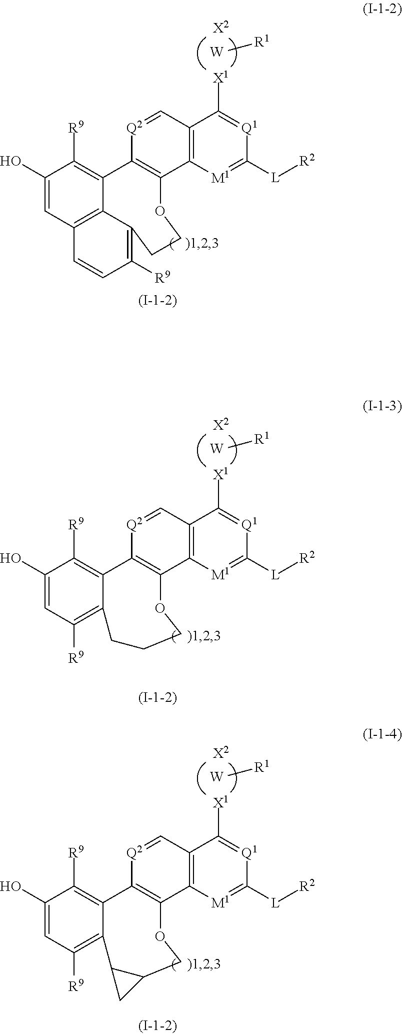

wherein R⁹ is hydrogen, halogen, substituted or unsubstituted C₁-C₃ alkyl, C₃-C₆ cycloalkyl, and the remaining variables are as defined for formula (I) or (II).

In some embodiments, the substituents of Q2 and Q3 in the compound of formula (I) are connected to form a saturated, partially saturated 5-6-membered aliphatic ring or aromatic heterocycle, as represented by formula (I-7), (I-8), (I-9), (I-10), (I-11), (I-12), (I-13), (I-14), (I-15), (I-16), (I-17), and (I-18):

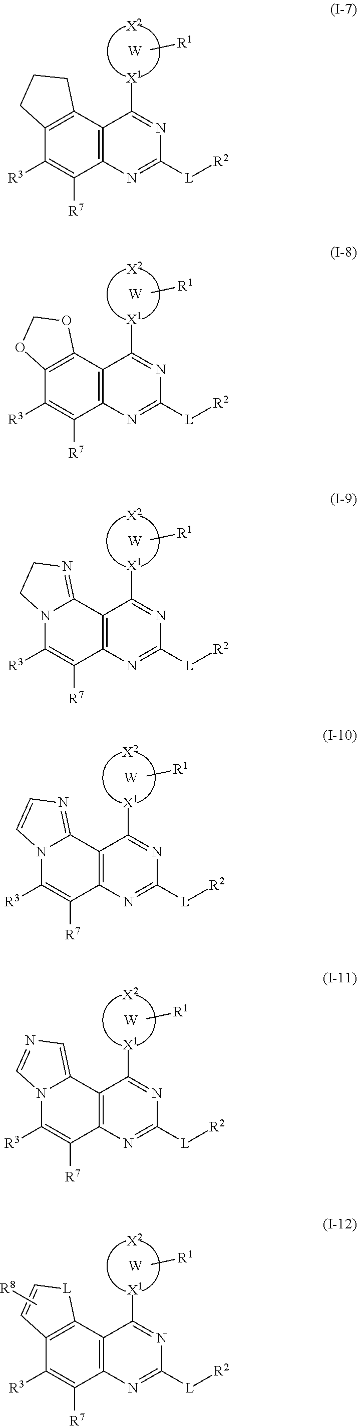

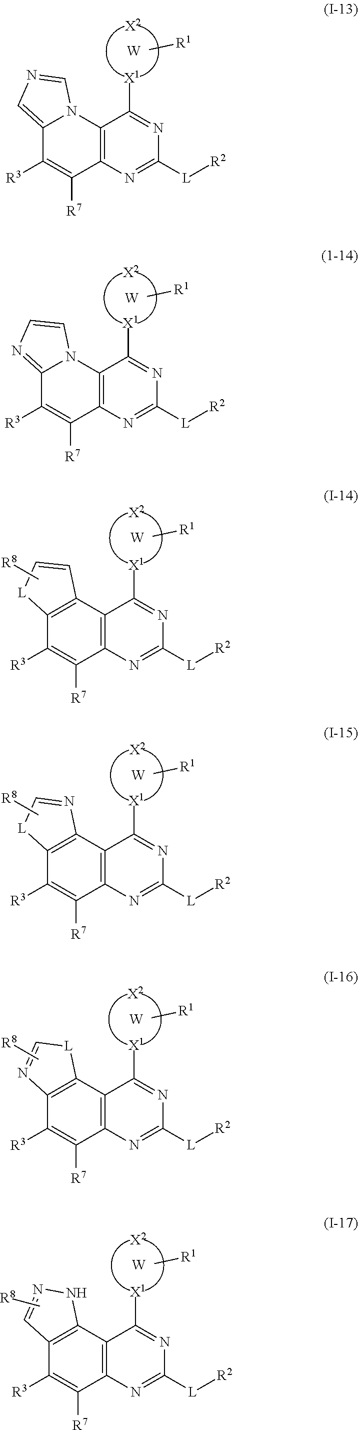

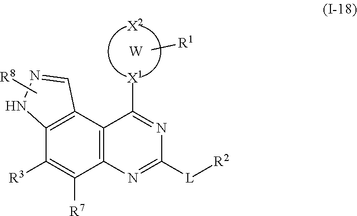

In some embodiments, the compound of formula (II) is as represented by formula (II-1):

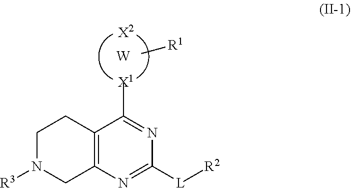

wherein:
L—R² is

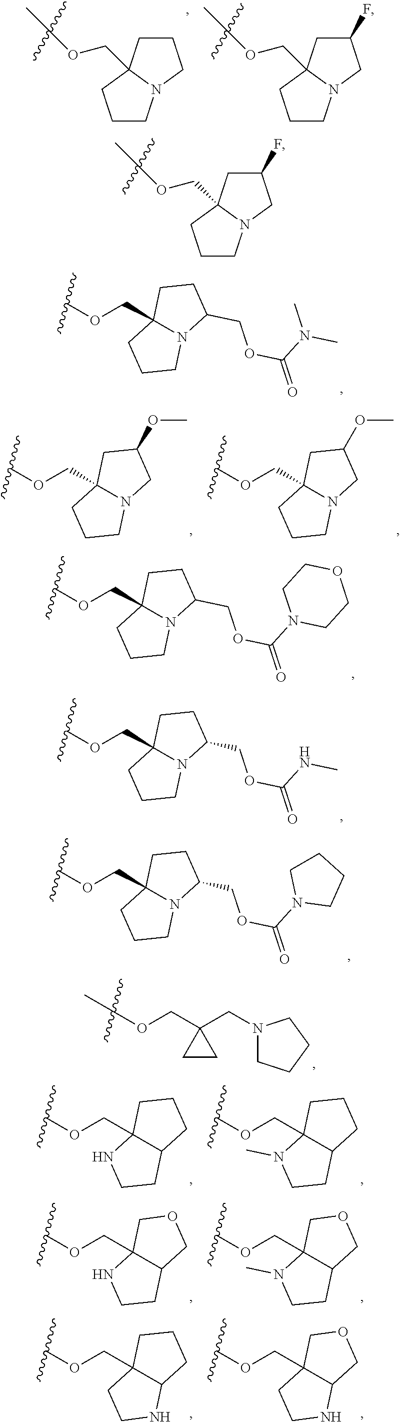

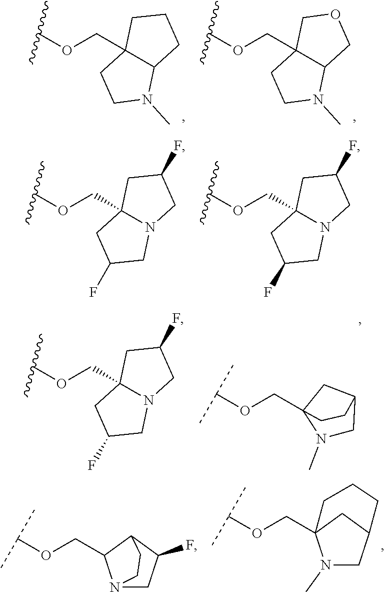

R³ is

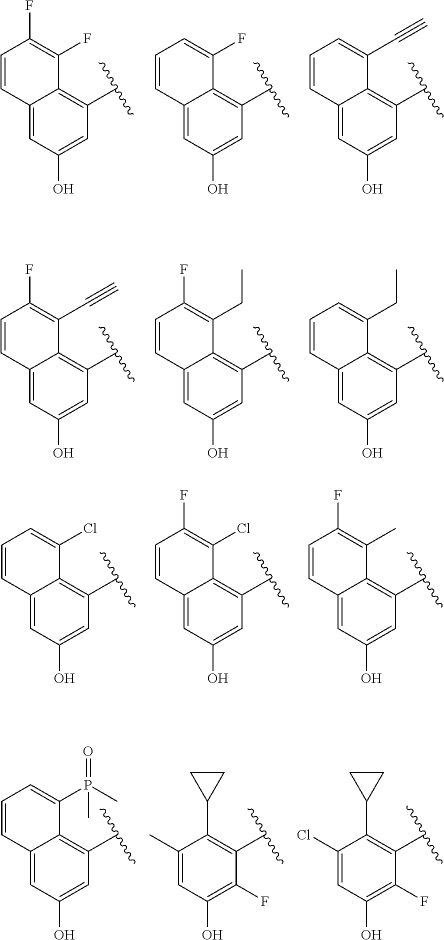

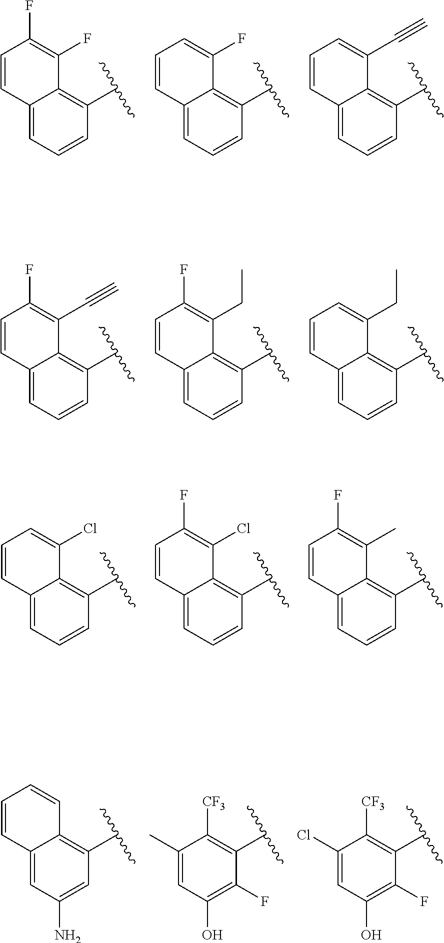

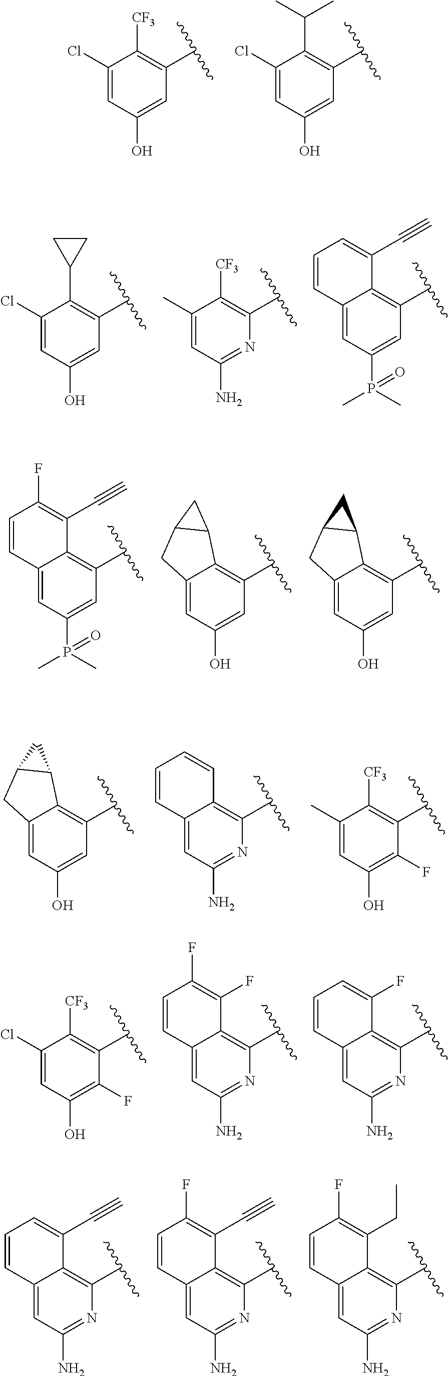

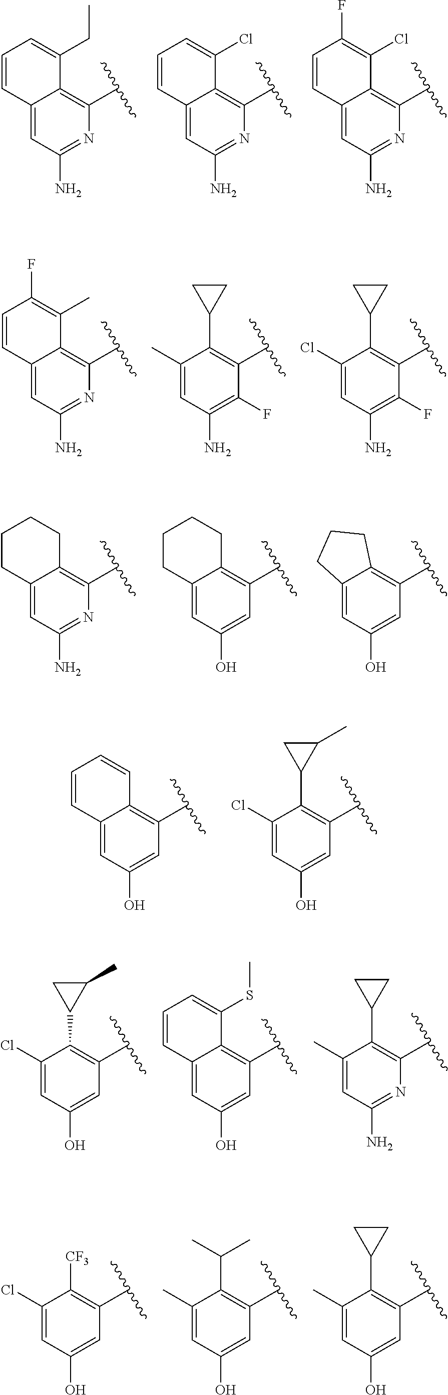

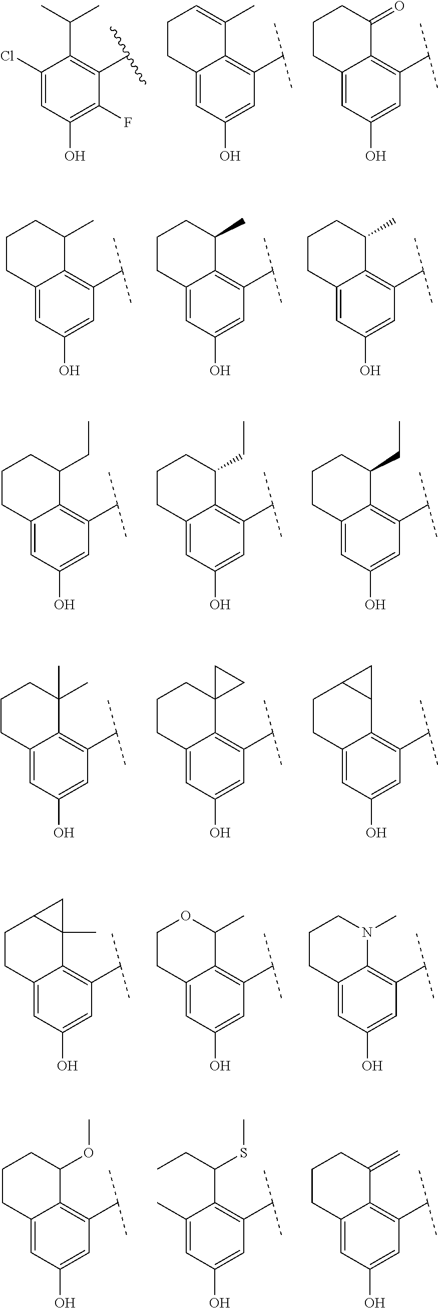

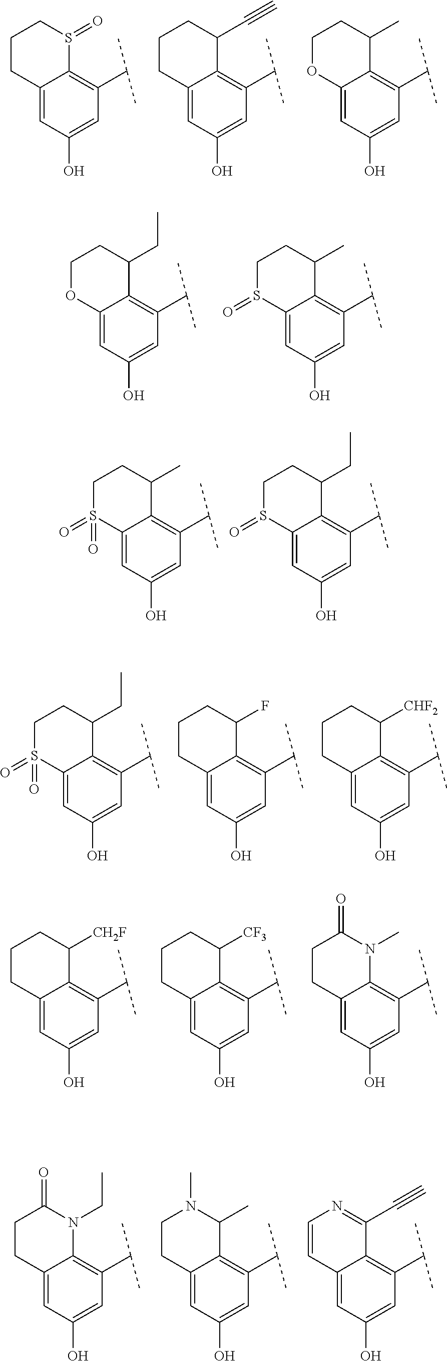

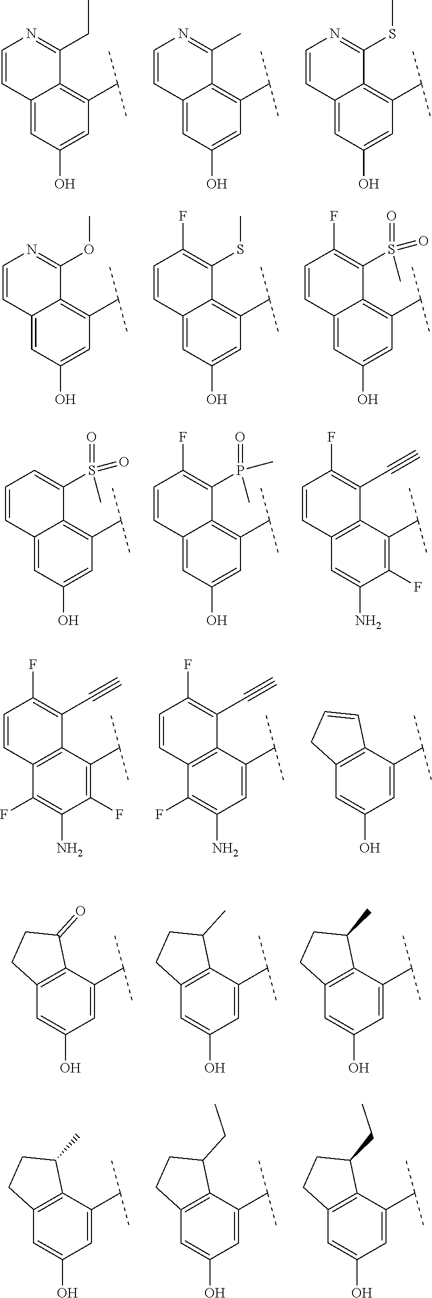

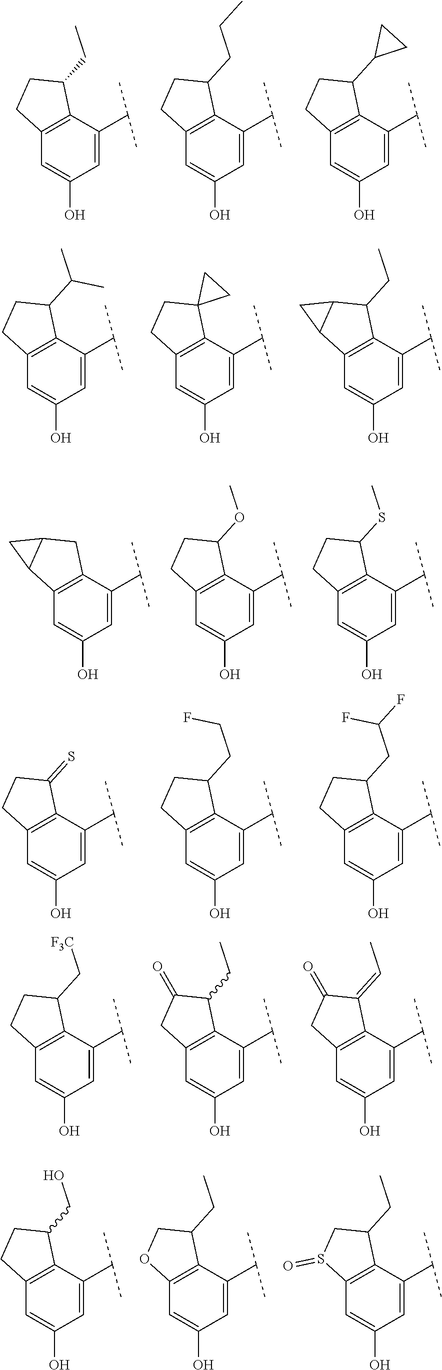

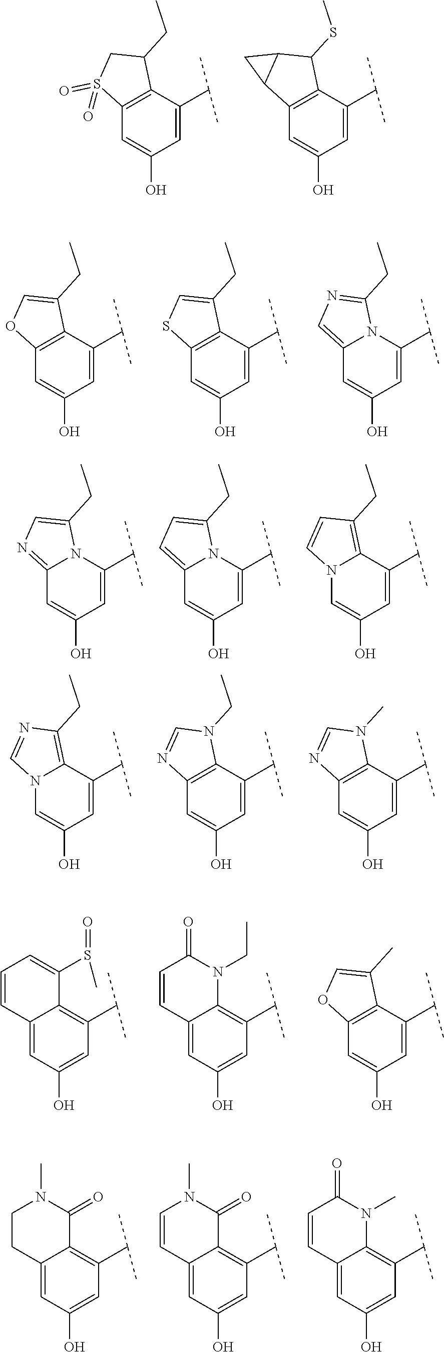

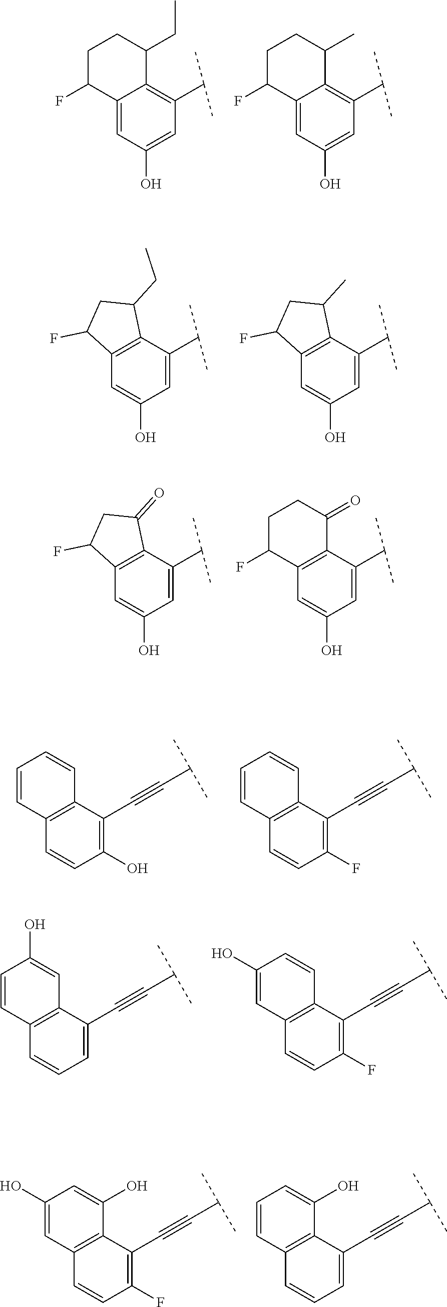

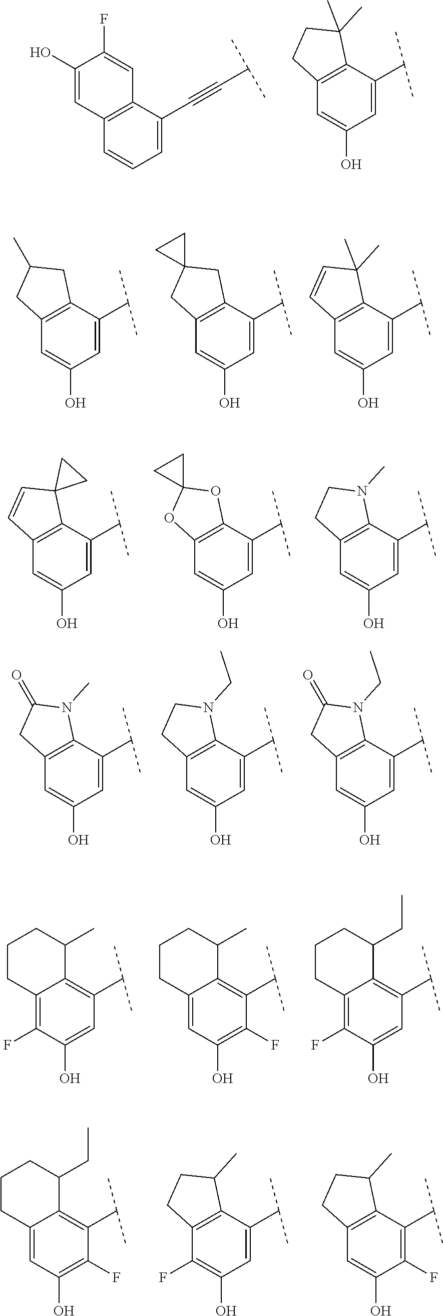

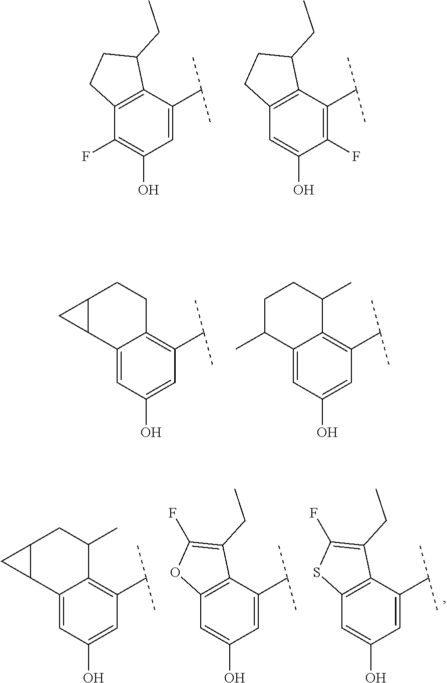

and the remaining variables are as defined for formula (I) or (II).

In some embodiments, the compound of formula (I) is

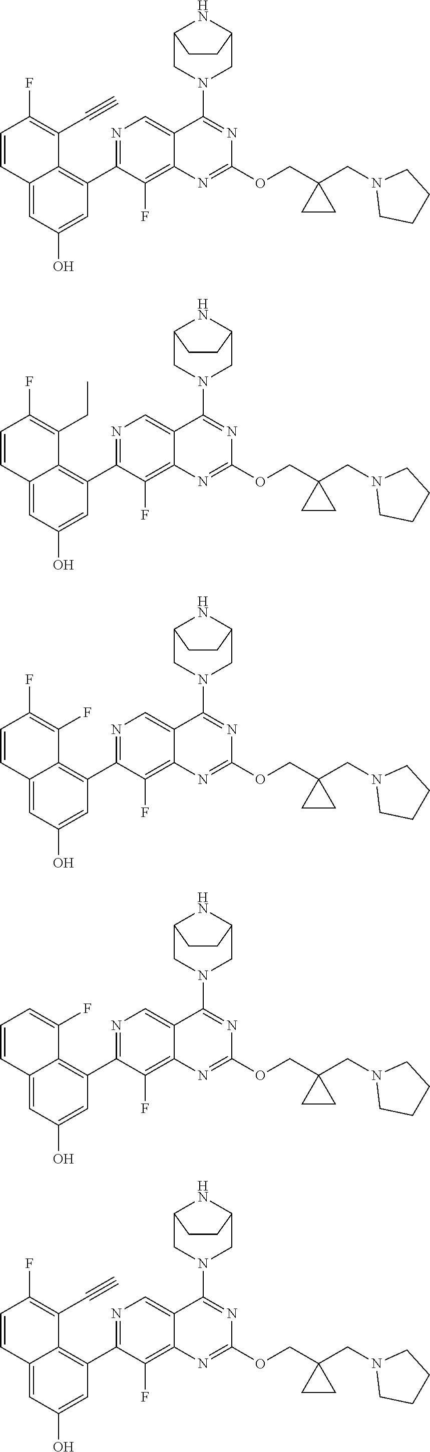 [SMILES: C#Cc1c(F)ccc2cc(O)cc(-c3ncc4c(N5CC6CCC(C5)N6)nc(OCC5(CN6CCCC6)CC5)nc4c3F)c12 ; CCc1c(F)ccc2cc(O)cc(-c3ncc4c(N5CC6CCC(C5)N6)nc(OCC5(CN6CCCC6)CC5)nc4c3F)c12 ; Oc1cc(-c2ncc3c(N4CC5CCC(C4)N5)nc(OCC4(CN5CCCC5)CC4)nc3c2F)c2c(F)c(F)ccc2c1 ; Oc1cc(-c2ncc3c(N4CC5CCC(C4)N5)nc(OCC4(CN5CCCC5)CC4)nc3c2F)c2c(F)cccc2c1 ; C#Cc1c(F)ccc2cc(O)cc(-c3ncc4c(N5CC6CCC(C5)N6)nc(OCC5(CN6CCCC6)CC5)nc4c3F)c12]

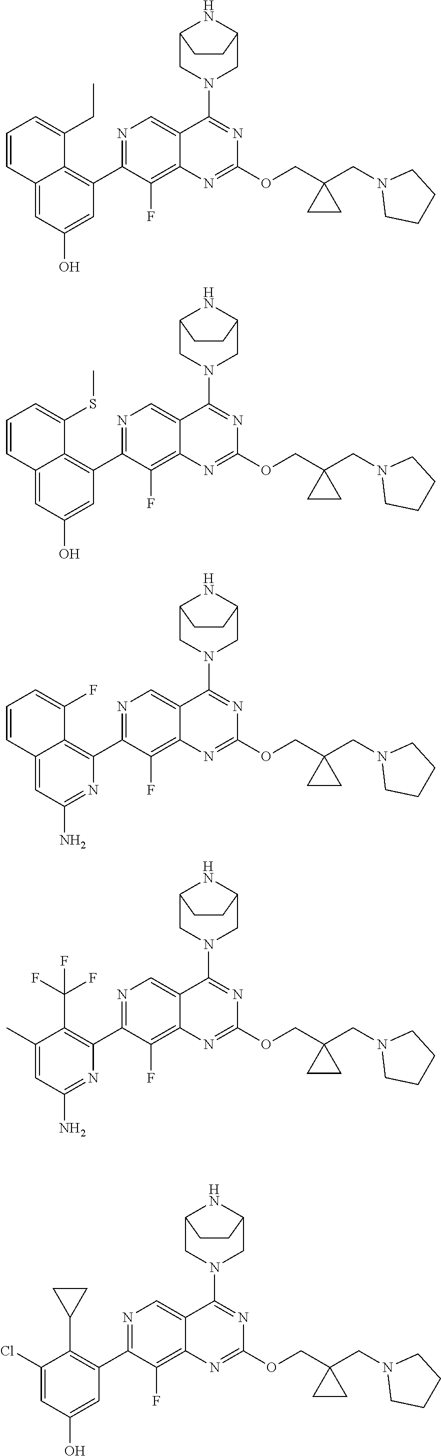 [SMILES: CCc1cccc2cc(O)cc(-c3ncc4c(N5CC6CCC(C5)N6)nc(OCC5(CN6CCCC6)CC5)nc4c3F)c12 ; CSc1cccc2cc(O)cc(-c3ncc4c(N5CC6CCC(C5)N6)nc(OCC5(CN6CCCC6)CC5)nc4c3F)c12 ; Nc1cc2cccc(F)c2c(-c2ncc3c(N4CC5CCC(C4)N5)nc(OCC4(CN5CCCC5)CC4)nc3c2F)n1 ; Cc1cc(N)nc(-c2ncc3c(N4CC5CCC(C4)N5)nc(OCC4(CN5CCCC5)CC4)nc3c2F)c1C(F)(F)F ; Oc1cc(Cl)c(C2CC2)c(-c2ncc3c(N4CC5CCC(C4)N5)nc(OCC4(CN5CCCC5)CC4)nc3c2F)c1]

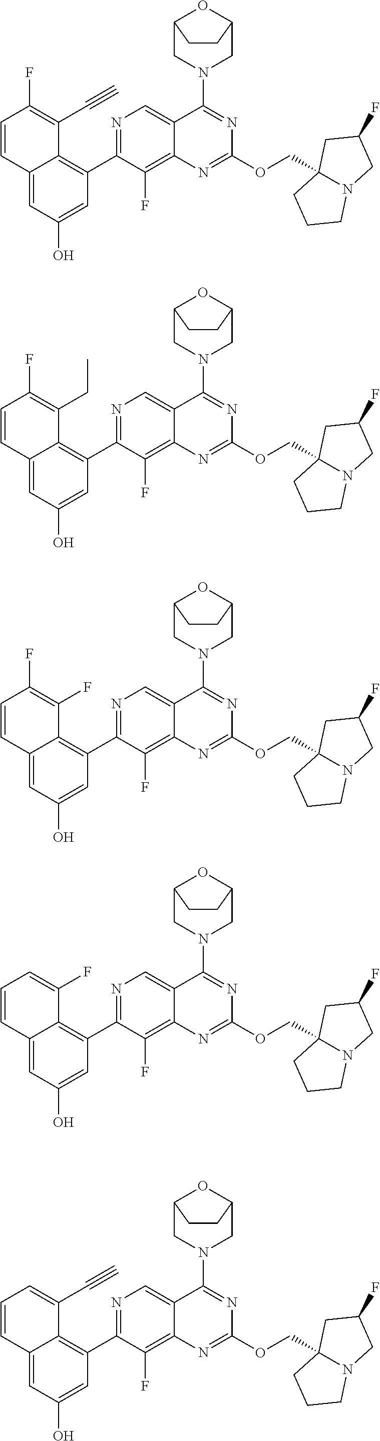 [SMILES: C#Cc1c(F)ccc2cc(O)cc(-c3ncc4c(N5CC6CCC(C5)O6)nc(OC[C@@]56CCCN5C[C@H](F)C6)nc4c3F)c12 ; CCc1c(F)ccc2cc(O)cc(-c3ncc4c(N5CC6CCC(C5)O6)nc(OC[C@@]56CCCN5C[C@H](F)C6)nc4c3F)c12 ; Oc1cc(-c2ncc3c(N4CC5CCC(C4)O5)nc(OC[C@@]45CCCN4C[C@H](F)C5)nc3c2F)c2c(F)c(F)ccc2c1 ; Oc1cc(-c2ncc3c(N4CC5CCC(C4)O5)nc(OC[C@@]45CCCN4C[C@H](F)C5)nc3c2F)c2c(F)cccc2c1 ; C#Cc1cccc2cc(O)cc(-c3ncc4c(N5CC6CCC(C5)O6)nc(OC[C@@]56CCCN5C[C@H](F)C6)nc4c3F)c12]

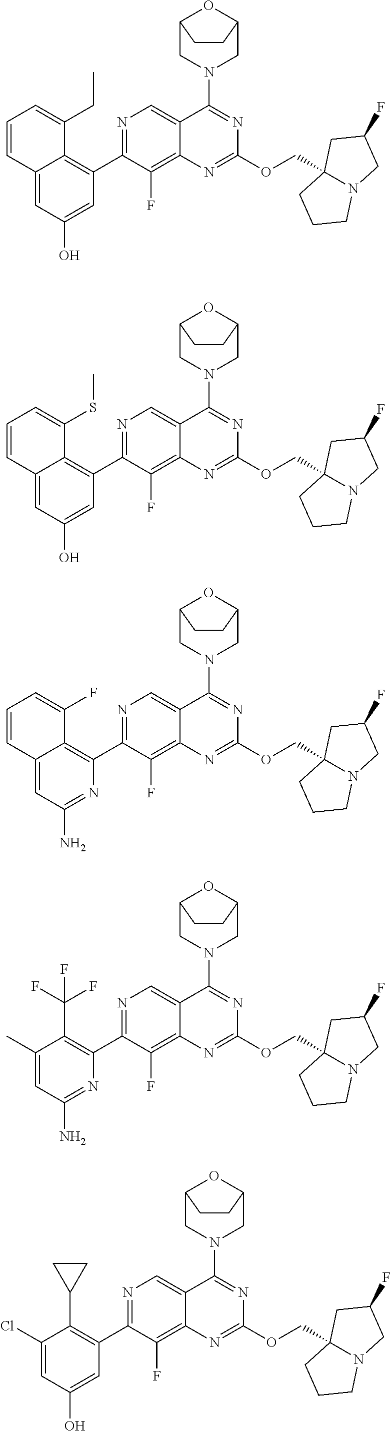 [SMILES: CCc1cccc2cc(O)cc(-c3ncc4c(N5CC6CCC(C5)O6)nc(OC[C@@]56CCCN5C[C@H](F)C6)nc4c3F)c12 ; CSc1cccc2cc(O)cc(-c3ncc4c(N5CC6CCC(C5)O6)nc(OC[C@@]56CCCN5C[C@H](F)C6)nc4c3F)c12 ; Nc1cc2cccc(F)c2c(-c2ncc3c(N4CC5CCC(C4)O5)nc(OC[C@@]45CCCN4C[C@H](F)C5)nc3c2F)n1 ; Cc1cc(N)nc(-c2ncc3c(N4CC5CCC(C4)O5)nc(OC[C@@]45CCCN4C[C@H](F)C5)nc3c2F)c1C(F)(F)F ; Oc1cc(Cl)c(C2CC2)c(-c2ncc3c(N4CC5CCC(C4)O5)nc(OC[C@@]45CCCN4C[C@H](F)C5)nc3c2F)c1]

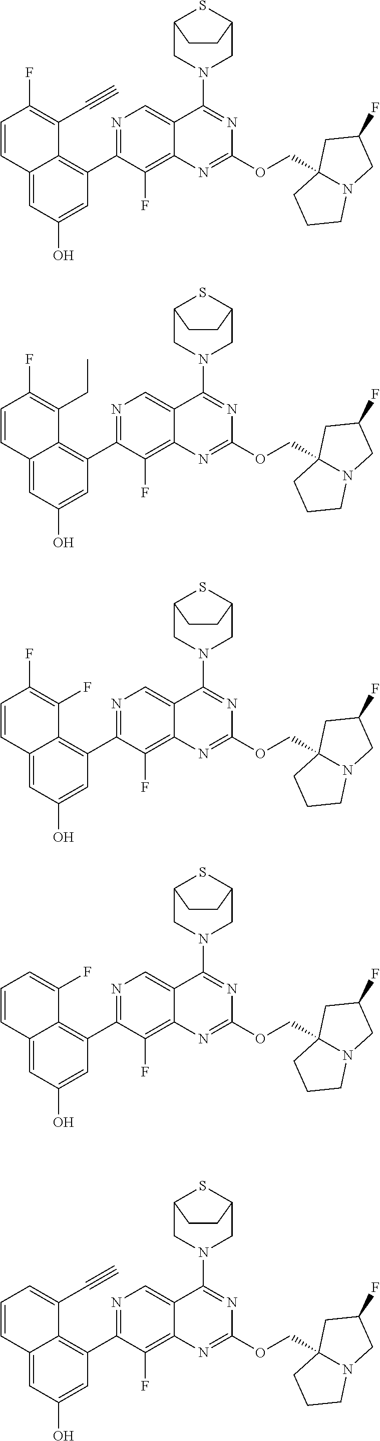 [SMILES: C#Cc1c(F)ccc2cc(O)cc(-c3ncc4c(N5CC6CCC(C5)S6)nc(OC[C@@]56CCCN5C[C@H](F)C6)nc4c3F)c12 ; CCc1c(F)ccc2cc(O)cc(-c3ncc4c(N5CC6CCC(C5)S6)nc(OC[C@@]56CCCN5C[C@H](F)C6)nc4c3F)c12 ; Oc1cc(-c2ncc3c(N4CC5CCC(C4)S5)nc(OC[C@@]45CCCN4C[C@H](F)C5)nc3c2F)c2c(F)c(F)ccc2c1 ; Oc1cc(-c2ncc3c(N4CC5CCC(C4)S5)nc(OC[C@@]45CCCN4C[C@H](F)C5)nc3c2F)c2c(F)cccc2c1 ; C#Cc1cccc2cc(O)cc(-c3ncc4c(N5CC6CCC(C5)S6)nc(OC[C@@]56CCCN5C[C@H](F)C6)nc4c3F)c12]

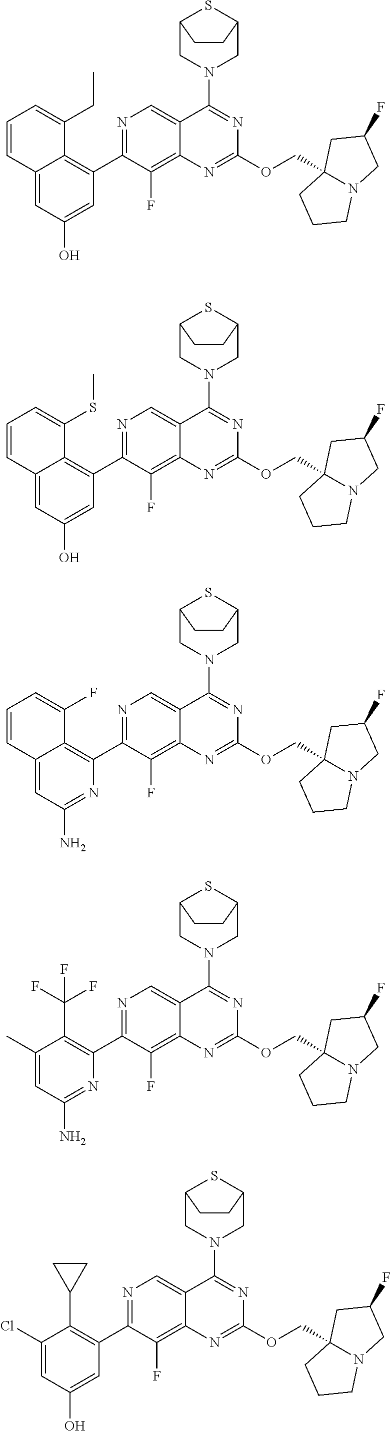 [SMILES: CCc1cccc2cc(O)cc(-c3ncc4c(N5CC6CCC(C5)S6)nc(OC[C@@]56CCCN5C[C@H](F)C6)nc4c3F)c12 ; CSc1cccc2cc(O)cc(-c3ncc4c(N5CC6CCC(C5)S6)nc(OC[C@@]56CCCN5C[C@H](F)C6)nc4c3F)c12 ; Nc1cc2cccc(F)c2c(-c2ncc3c(N4CC5CCC(C4)S5)nc(OC[C@@]45CCCN4C[C@H](F)C5)nc3c2F)n1 ; Cc1cc(N)nc(-c2ncc3c(N4CC5CCC(C4)S5)nc(OC[C@@]45CCCN4C[C@H](F)C5)nc3c2F)c1C(F)(F)F ; Oc1cc(Cl)c(C2CC2)c(-c2ncc3c(N4CC5CCC(C4)S5)nc(OC[C@@]45CCCN4C[C@H](F)C5)nc3c2F)c1]

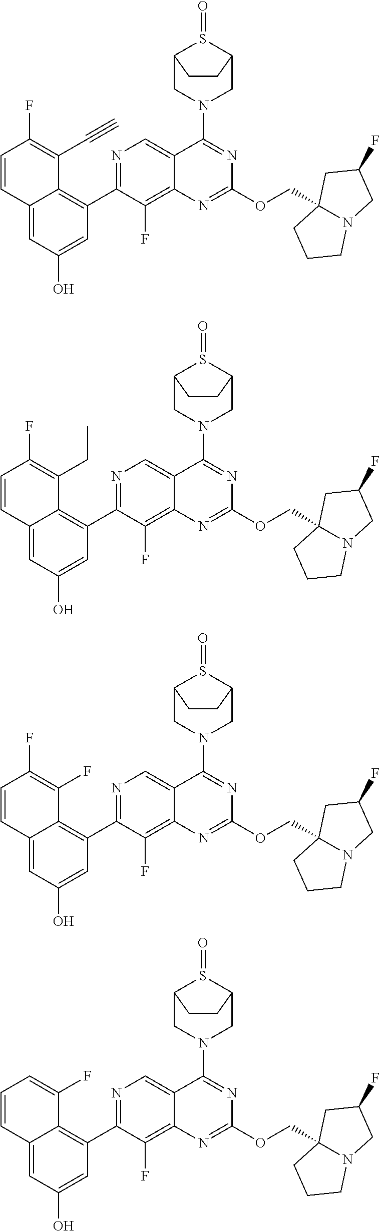 [SMILES: C#Cc1c(F)ccc2cc(O)cc(-c3ncc4c(N5CC6CCC(C5)S6=O)nc(OC[C@@]56CCCN5C[C@H](F)C6)nc4c3F)c12 ; CCc1c(F)ccc2cc(O)cc(-c3ncc4c(N5CC6CCC(C5)S6=O)nc(OC[C@@]56CCCN5C[C@H](F)C6)nc4c3F)c12 ; O=S1C2CCC1CN(c1nc(OC[C@@]34CCCN3C[C@H](F)C4)nc3c(F)c(-c4cc(O)cc5ccc(F)c(F)c45)ncc13)C2 ; O=S1C2CCC1CN(c1nc(OC[C@@]34CCCN3C[C@H](F)C4)nc3c(F)c(-c4cc(O)cc5cccc(F)c45)ncc13)C2]

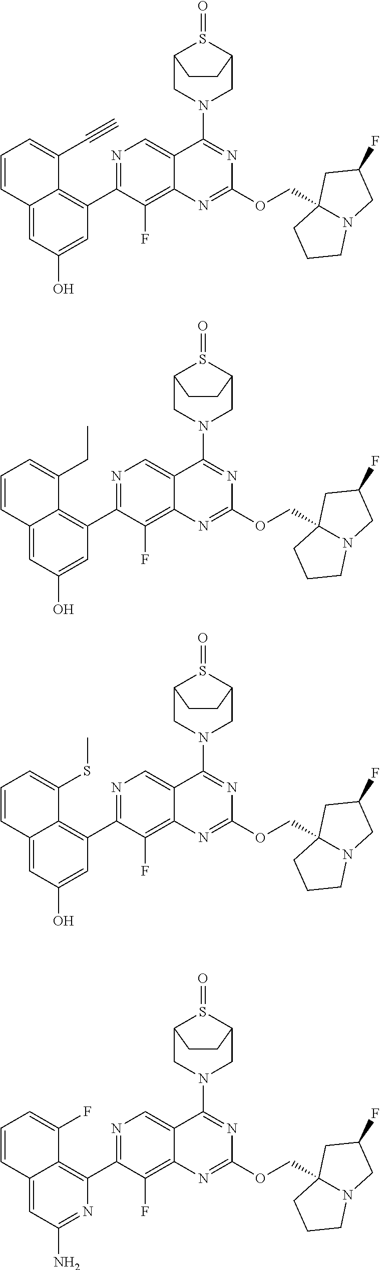 [SMILES: C#Cc1cccc2cc(O)cc(-c3ncc4c(N5CC6CCC(C5)S6=O)nc(OC[C@@]56CCCN5C[C@H](F)C6)nc4c3F)c12 ; CCc1cccc2cc(O)cc(-c3ncc4c(N5CC6CCC(C5)S6=O)nc(OC[C@@]56CCCN5C[C@H](F)C6)nc4c3F)c12 ; CSc1cccc2cc(O)cc(-c3ncc4c(N5CC6CCC(C5)S6=O)nc(OC[C@@]56CCCN5C[C@H](F)C6)nc4c3F)c12 ; Nc1cc2cccc(F)c2c(-c2ncc3c(N4CC5CCC(C4)S5=O)nc(OC[C@@]45CCCN4C[C@H](F)C5)nc3c2F)n1]

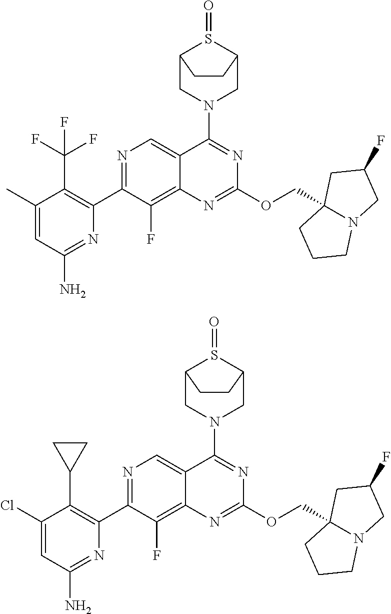 [SMILES: Cc1cc(N)nc(-c2ncc3c(N4CC5CCC(C4)S5=O)nc(OC[C@@]45CCCN4C[C@H](F)C5)nc3c2F)c1C(F)(F)F ; Nc1cc(Cl)c(C2CC2)c(-c2ncc3c(N4CC5CCC(C4)S5=O)nc(OC[C@@]45CCCN4C[C@H](F)C5)nc3c2F)n1]

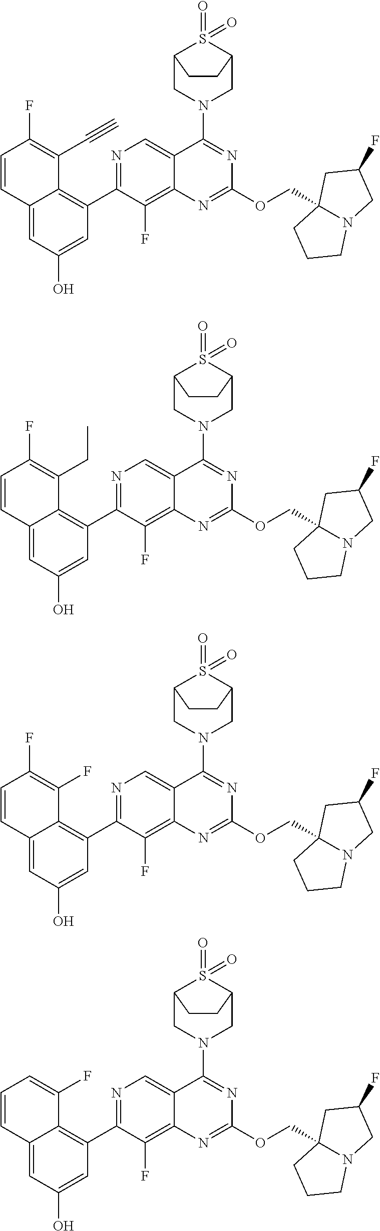 [SMILES: C#Cc1c(F)ccc2cc(O)cc(-c3ncc4c(N5CC6CCC(C5)S6(=O)=O)nc(OC[C@@]56CCCN5C[C@H](F)C6)nc4c3F)c12 ; CCc1c(F)ccc2cc(O)cc(-c3ncc4c(N5CC6CCC(C5)S6(=O)=O)nc(OC[C@@]56CCCN5C[C@H](F)C6)nc4c3F)c12 ; O=S1(=O)C2CCC1CN(c1nc(OC[C@@]34CCCN3C[C@H](F)C4)nc3c(F)c(-c4cc(O)cc5ccc(F)c(F)c45)ncc13)C2 ; O=S1(=O)C2CCC1CN(c1nc(OC[C@@]34CCCN3C[C@H](F)C4)nc3c(F)c(-c4cc(O)cc5cccc(F)c45)ncc13)C2]

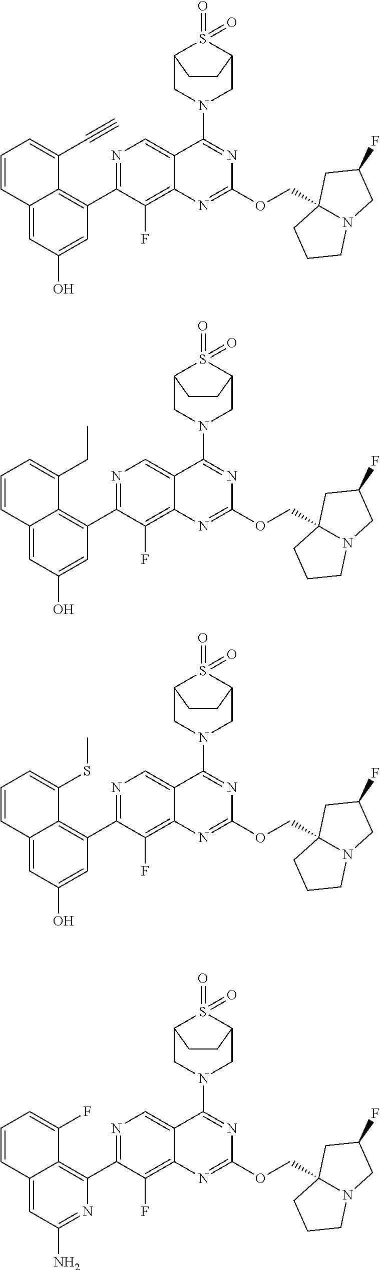 [SMILES: C#Cc1cccc2cc(O)cc(-c3ncc4c(N5CC6CCC(C5)S6(=O)=O)nc(OC[C@@]56CCCN5C[C@H](F)C6)nc4c3F)c12 ; CCc1cccc2cc(O)cc(-c3ncc4c(N5CC6CCC(C5)S6(=O)=O)nc(OC[C@@]56CCCN5C[C@H](F)C6)nc4c3F)c12 ; CSc1cccc2cc(O)cc(-c3ncc4c(N5CC6CCC(C5)S6(=O)=O)nc(OC[C@@]56CCCN5C[C@H](F)C6)nc4c3F)c12 ; Nc1cc2cccc(F)c2c(-c2ncc3c(N4CC5CCC(C4)S5(=O)=O)nc(OC[C@@]45CCCN4C[C@H](F)C5)nc3c2F)n1]

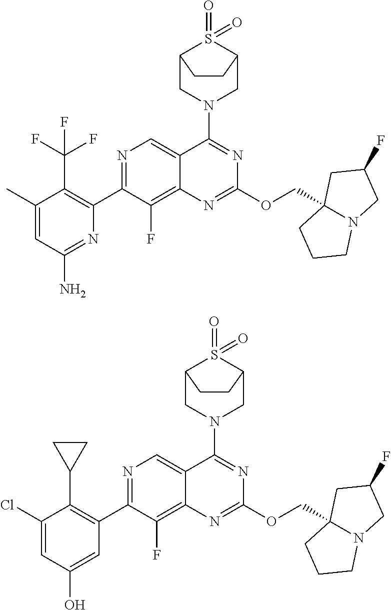 [SMILES: Cc1cc(N)nc(-c2ncc3c(N4CC5CCC(C4)S5(=O)=O)nc(OC[C@@]45CCCN4C[C@H](F)C5)nc3c2F)c1C(F)(F)F ; O=S1(=O)C2CCC1CN(c1nc(OC[C@@]34CCCN3C[C@H](F)C4)nc3c(F)c(-c4cc(O)cc(Cl)c4C4CC4)ncc13)C2]

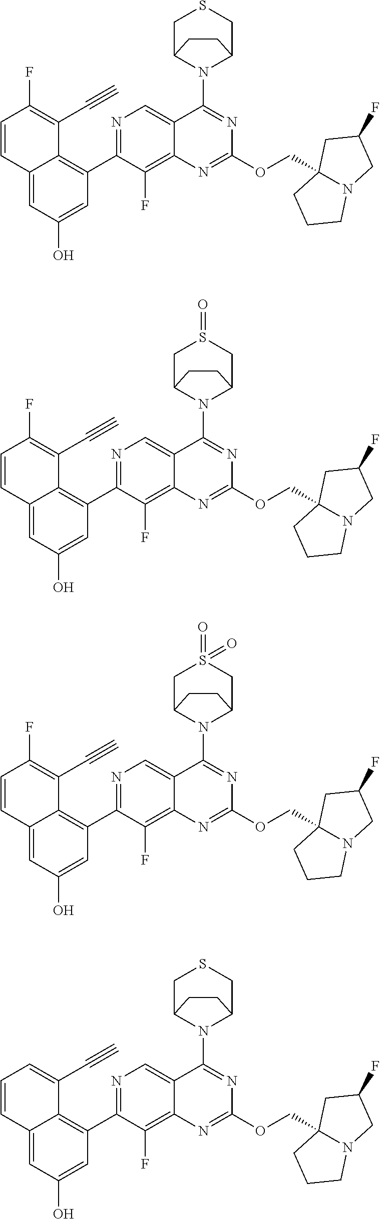 [SMILES: C#Cc1c(F)ccc2cc(O)cc(-c3ncc4c(N5C6CCC5CSC6)nc(OC[C@@]56CCCN5C[C@H](F)C6)nc4c3F)c12 ; C#Cc1c(F)ccc2cc(O)cc(-c3ncc4c(N5C6CCC5CS(=O)C6)nc(OC[C@@]56CCCN5C[C@H](F)C6)nc4c3F)c12 ; C#Cc1c(F)ccc2cc(O)cc(-c3ncc4c(N5C6CCC5CS(=O)(=O)C6)nc(OC[C@@]56CCCN5C[C@H](F)C6)nc4c3F)c12 ; C#Cc1cccc2cc(O)cc(-c3ncc4c(N5C6CCC5CSC6)nc(OC[C@@]56CCCN5C[C@H](F)C6)nc4c3F)c12]

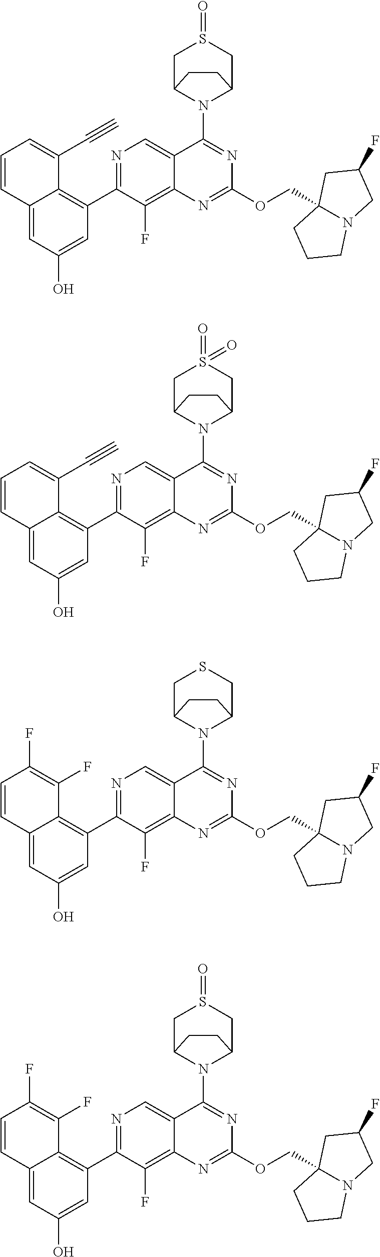 [SMILES: C#Cc1cccc2cc(O)cc(-c3ncc4c(N5C6CCC5CS(=O)C6)nc(OC[C@@]56CCCN5C[C@H](F)C6)nc4c3F)c12 ; C#Cc1cccc2cc(O)cc(-c3ncc4c(N5C6CCC5CS(=O)(=O)C6)nc(OC[C@@]56CCCN5C[C@H](F)C6)nc4c3F)c12 ; Oc1cc(-c2ncc3c(N4C5CCC4CSC5)nc(OC[C@@]45CCCN4C[C@H](F)C5)nc3c2F)c2c(F)c(F)ccc2c1 ; O=S1CC2CCC(C1)N2c1nc(OC[C@@]23CCCN2C[C@H](F)C3)nc2c(F)c(-c3cc(O)cc4ccc(F)c(F)c34)ncc12]

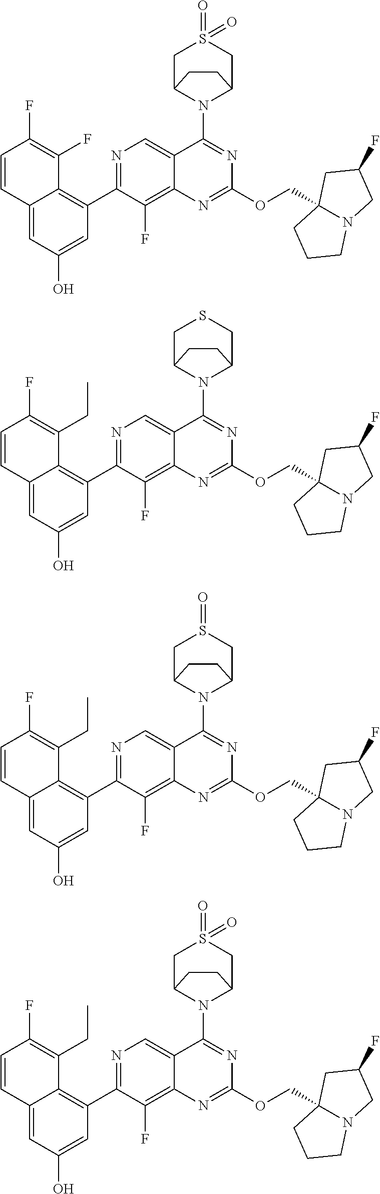 [SMILES: O=S1(=O)CC2CCC(C1)N2c1nc(OC[C@@]23CCCN2C[C@H](F)C3)nc2c(F)c(-c3cc(O)cc4ccc(F)c(F)c34)ncc12 ; CCc1c(F)ccc2cc(O)cc(-c3ncc4c(N5C6CCC5CSC6)nc(OC[C@@]56CCCN5C[C@H](F)C6)nc4c3F)c12 ; CCc1c(F)ccc2cc(O)cc(-c3ncc4c(N5C6CCC5CS(=O)C6)nc(OC[C@@]56CCCN5C[C@H](F)C6)nc4c3F)c12 ; CCc1c(F)ccc2cc(O)cc(-c3ncc4c(N5C6CCC5CS(=O)(=O)C6)nc(OC[C@@]56CCCN5C[C@H](F)C6)nc4c3F)c12]

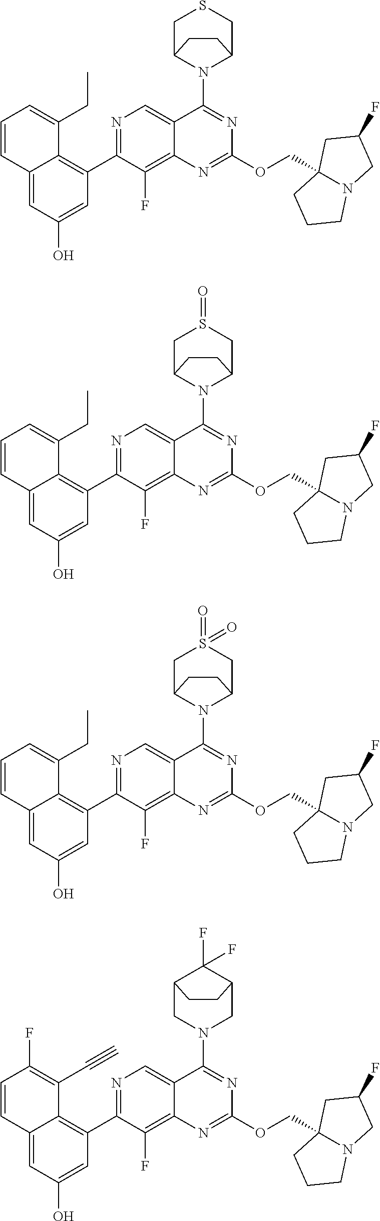 [SMILES: CCc1cccc2cc(O)cc(-c3ncc4c(N5C6CCC5CSC6)nc(OC[C@@]56CCCN5C[C@H](F)C6)nc4c3F)c12 ; CCc1cccc2cc(O)cc(-c3ncc4c(N5C6CCC5CS(=O)C6)nc(OC[C@@]56CCCN5C[C@H](F)C6)nc4c3F)c12 ; CCc1cccc2cc(O)cc(-c3ncc4c(N5C6CCC5CS(=O)(=O)C6)nc(OC[C@@]56CCCN5C[C@H](F)C6)nc4c3F)c12 ; C#Cc1c(F)ccc2cc(O)cc(-c3ncc4c(N5CC6CCC(C5)C6(F)F)nc(OC[C@@]56CCCN5C[C@H](F)C6)nc4c3F)c12]

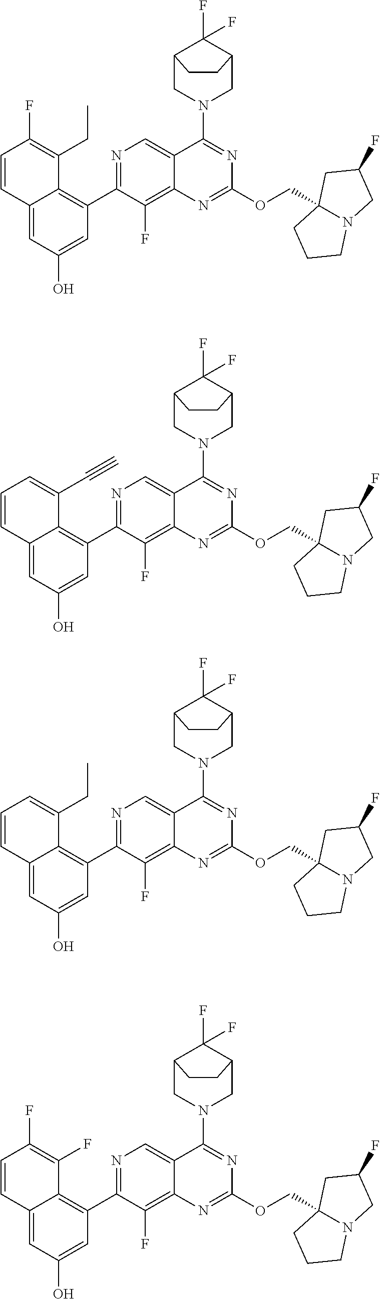 [SMILES: CCc1c(F)ccc2cc(O)cc(-c3ncc4c(N5CC6CCC(C5)C6(F)F)nc(OC[C@@]56CCCN5C[C@H](F)C6)nc4c3F)c12 ; C#Cc1cccc2cc(O)cc(-c3ncc4c(N5CC6CCC(C5)C6(F)F)nc(OC[C@@]56CCCN5C[C@H](F)C6)nc4c3F)c12 ; CCc1cccc2cc(O)cc(-c3ncc4c(N5CC6CCC(C5)C6(F)F)nc(OC[C@@]56CCCN5C[C@H](F)C6)nc4c3F)c12 ; Oc1cc(-c2ncc3c(N4CC5CCC(C4)C5(F)F)nc(OC[C@@]45CCCN4C[C@H](F)C5)nc3c2F)c2c(F)c(F)ccc2c1]

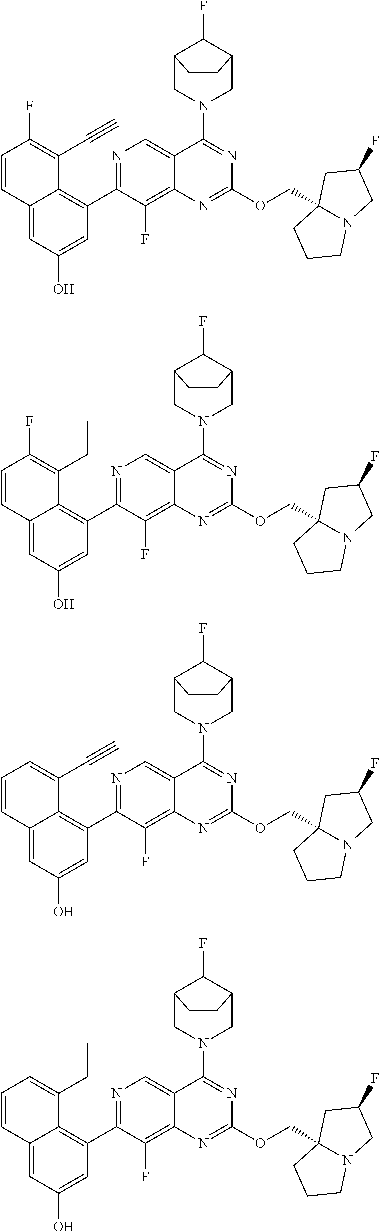 [SMILES: C#Cc1c(F)ccc2cc(O)cc(-c3ncc4c(N5CC6CCC(C5)C6F)nc(OC[C@@]56CCCN5C[C@H](F)C6)nc4c3F)c12 ; CCc1c(F)ccc2cc(O)cc(-c3ncc4c(N5CC6CCC(C5)C6F)nc(OC[C@@]56CCCN5C[C@H](F)C6)nc4c3F)c12 ; C#Cc1cccc2cc(O)cc(-c3ncc4c(N5CC6CCC(C5)C6F)nc(OC[C@@]56CCCN5C[C@H](F)C6)nc4c3F)c12 ; CCc1cccc2cc(O)cc(-c3ncc4c(N5CC6CCC(C5)C6F)nc(OC[C@@]56CCCN5C[C@H](F)C6)nc4c3F)c12]

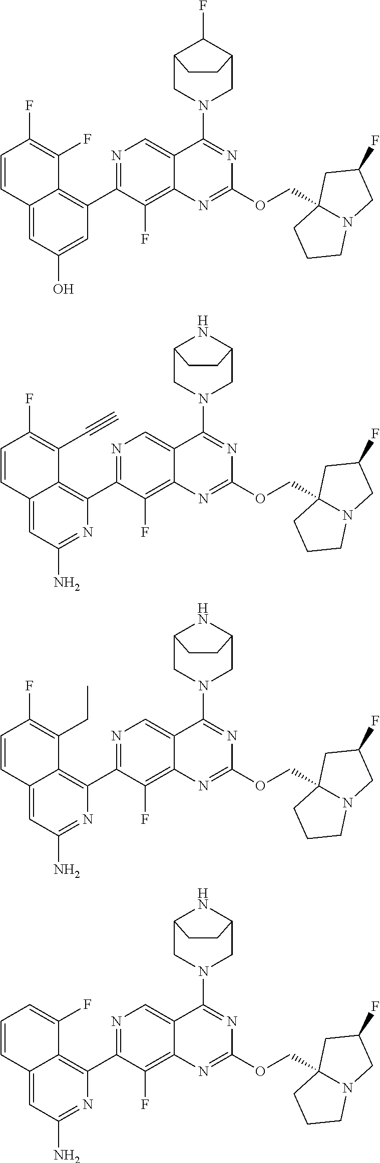 [SMILES: Oc1cc(-c2ncc3c(N4CC5CCC(C4)C5F)nc(OC[C@@]45CCCN4C[C@H](F)C5)nc3c2F)c2c(F)c(F)ccc2c1 ; C#Cc1c(F)ccc2cc(N)nc(-c3ncc4c(N5CC6CCC(C5)N6)nc(OC[C@@]56CCCN5C[C@H](F)C6)nc4c3F)c12 ; CCc1c(F)ccc2cc(N)nc(-c3ncc4c(N5CC6CCC(C5)N6)nc(OC[C@@]56CCCN5C[C@H](F)C6)nc4c3F)c12 ; Nc1cc2cccc(F)c2c(-c2ncc3c(N4CC5CCC(C4)N5)nc(OC[C@@]45CCCN4C[C@H](F)C5)nc3c2F)n1]

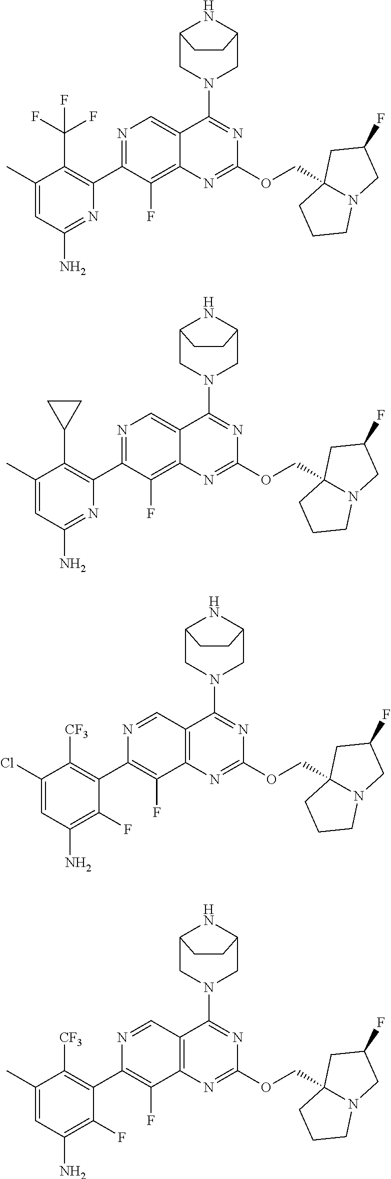 [SMILES: Cc1cc(N)nc(-c2ncc3c(N4CC5CCC(C4)N5)nc(OC[C@@]45CCCN4C[C@H](F)C5)nc3c2F)c1C(F)(F)F ; Cc1cc(N)nc(-c2ncc3c(N4CC5CCC(C4)N5)nc(OC[C@@]45CCCN4C[C@H](F)C5)nc3c2F)c1C1CC1 ; Nc1cc(Cl)c(C(F)(F)F)c(-c2ncc3c(N4CC5CCC(C4)N5)nc(OC[C@@]45CCCN4C[C@H](F)C5)nc3c2F)c1F ; Cc1cc(N)c(F)c(-c2ncc3c(N4CC5CCC(C4)N5)nc(OC[C@@]45CCCN4C[C@H](F)C5)nc3c2F)c1C(F)(F)F]

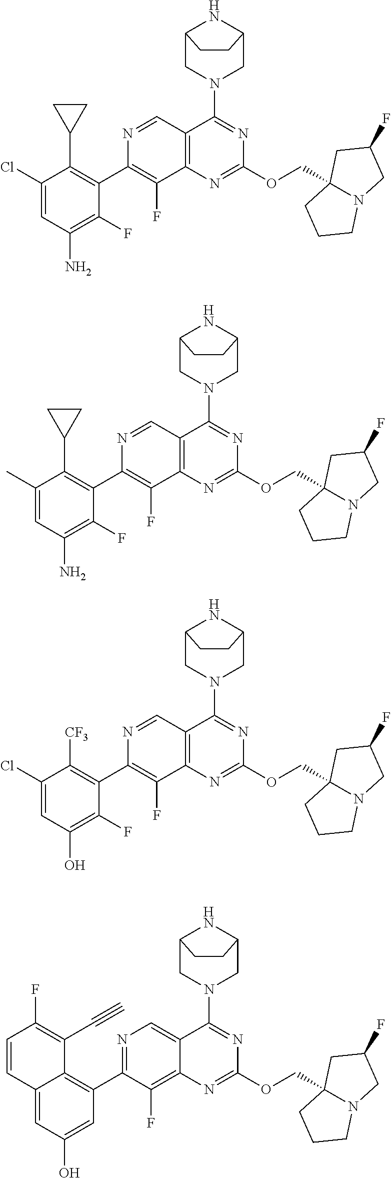 [SMILES: Nc1cc(Cl)c(C2CC2)c(-c2ncc3c(N4CC5CCC(C4)N5)nc(OC[C@@]45CCCN4C[C@H](F)C5)nc3c2F)c1F ; Cc1cc(N)c(F)c(-c2ncc3c(N4CC5CCC(C4)N5)nc(OC[C@@]45CCCN4C[C@H](F)C5)nc3c2F)c1C1CC1 ; Oc1cc(Cl)c(C(F)(F)F)c(-c2ncc3c(N4CC5CCC(C4)N5)nc(OC[C@@]45CCCN4C[C@H](F)C5)nc3c2F)c1F ; C#Cc1c(F)ccc2cc(O)cc(-c3ncc4c(N5CC6CCC(C5)N6)nc(OC[C@@]56CCCN5C[C@H](F)C6)nc4c3F)c12]

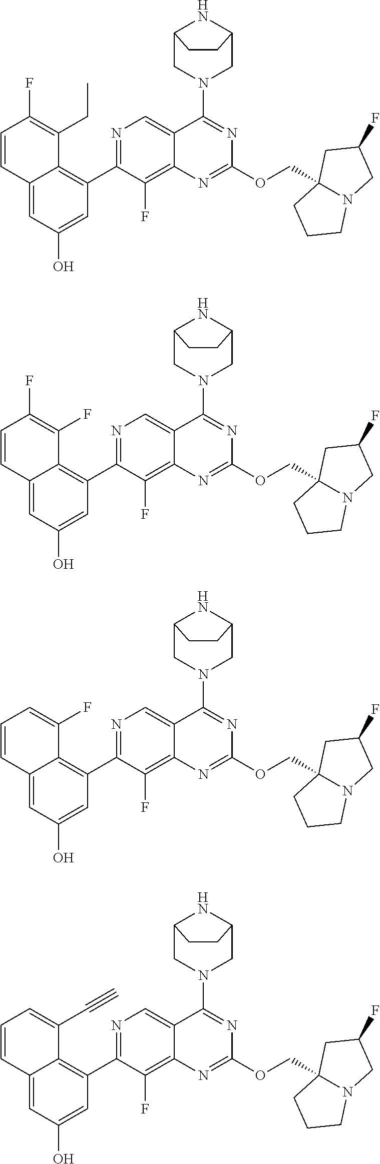 [SMILES: CCc1c(F)ccc2cc(O)cc(-c3ncc4c(N5CC6CCC(C5)N6)nc(OC[C@@]56CCCN5C[C@H](F)C6)nc4c3F)c12 ; Oc1cc(-c2ncc3c(N4CC5CCC(C4)N5)nc(OC[C@@]45CCCN4C[C@H](F)C5)nc3c2F)c2c(F)c(F)ccc2c1 ; Oc1cc(-c2ncc3c(N4CC5CCC(C4)N5)nc(OC[C@@]45CCCN4C[C@H](F)C5)nc3c2F)c2c(F)cccc2c1 ; C#Cc1cccc2cc(O)cc(-c3ncc4c(N5CC6CCC(C5)N6)nc(OC[C@@]56CCCN5C[C@H](F)C6)nc4c3F)c12]

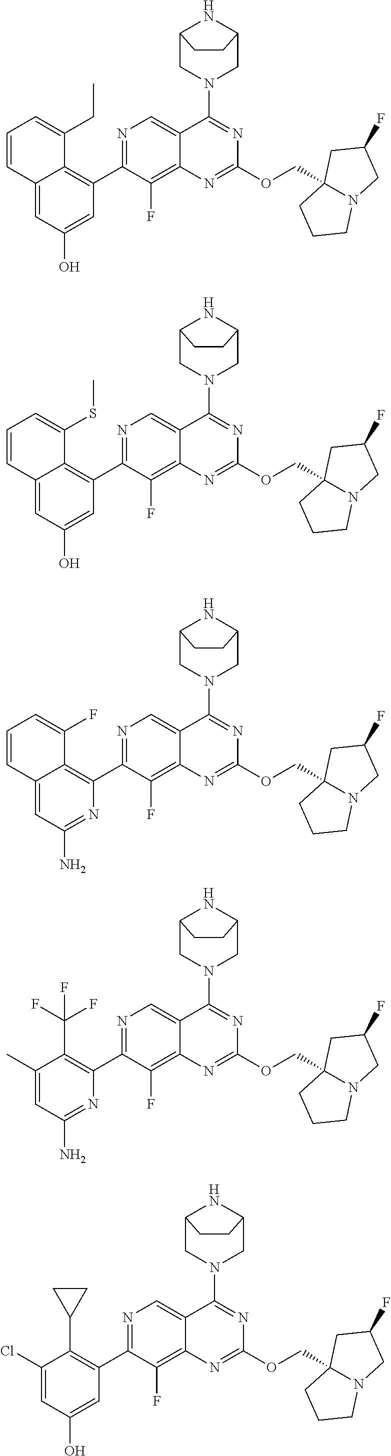 [SMILES: CCc1cccc2cc(O)cc(-c3ncc4c(N5CC6CCC(C5)N6)nc(OC[C@@]56CCCN5C[C@H](F)C6)nc4c3F)c12 ; CSc1cccc2cc(O)cc(-c3ncc4c(N5CC6CCC(C5)N6)nc(OC[C@@]56CCCN5C[C@H](F)C6)nc4c3F)c12 ; Nc1cc2cccc(F)c2c(-c2ncc3c(N4CC5CCC(C4)N5)nc(OC[C@@]45CCCN4C[C@H](F)C5)nc3c2F)n1 ; Cc1cc(N)nc(-c2ncc3c(N4CC5CCC(C4)N5)nc(OC[C@@]45CCCN4C[C@H](F)C5)nc3c2F)c1C(F)(F)F ; Oc1cc(Cl)c(C2CC2)c(-c2ncc3c(N4CC5CCC(C4)N5)nc(OC[C@@]45CCCN4C[C@H](F)C5)nc3c2F)c1]

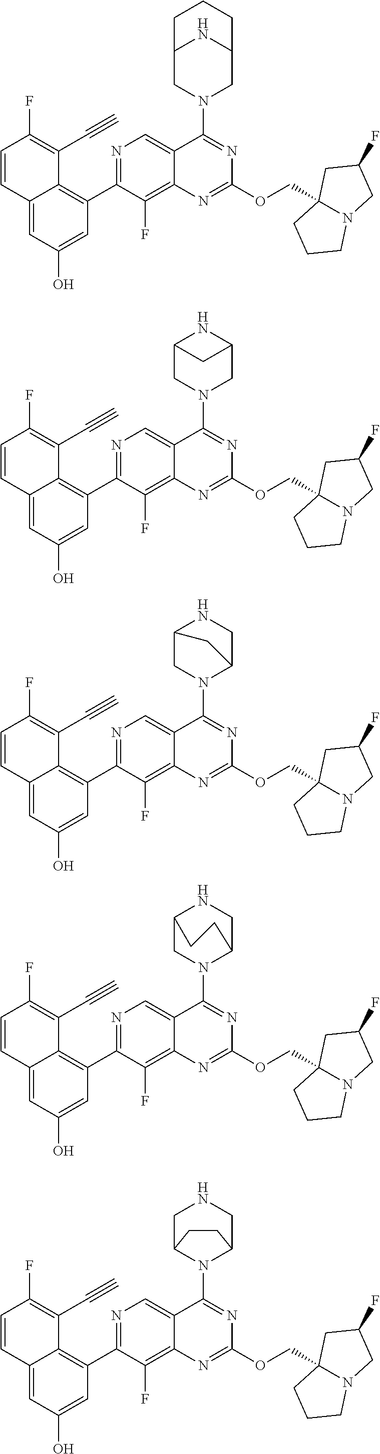 [SMILES: C#Cc1c(F)ccc2cc(O)cc(-c3ncc4c(N5CC6CCCC(C5)N6)nc(OC[C@@]56CCCN5C[C@H](F)C6)nc4c3F)c12 ; C#Cc1c(F)ccc2cc(O)cc(-c3ncc4c(N5CC6CC(C5)N6)nc(OC[C@@]56CCCN5C[C@H](F)C6)nc4c3F)c12 ; C#Cc1c(F)ccc2cc(O)cc(-c3ncc4c(N5CC6CC5CN6)nc(OC[C@@]56CCCN5C[C@H](F)C6)nc4c3F)c12 ; C#Cc1c(F)ccc2cc(O)cc(-c3ncc4c(N5CC6CCC5CN6)nc(OC[C@@]56CCCN5C[C@H](F)C6)nc4c3F)c12 ; C#Cc1c(F)ccc2cc(O)cc(-c3ncc4c(N5C6CCC5CNC6)nc(OC[C@@]56CCCN5C[C@H](F)C6)nc4c3F)c12]

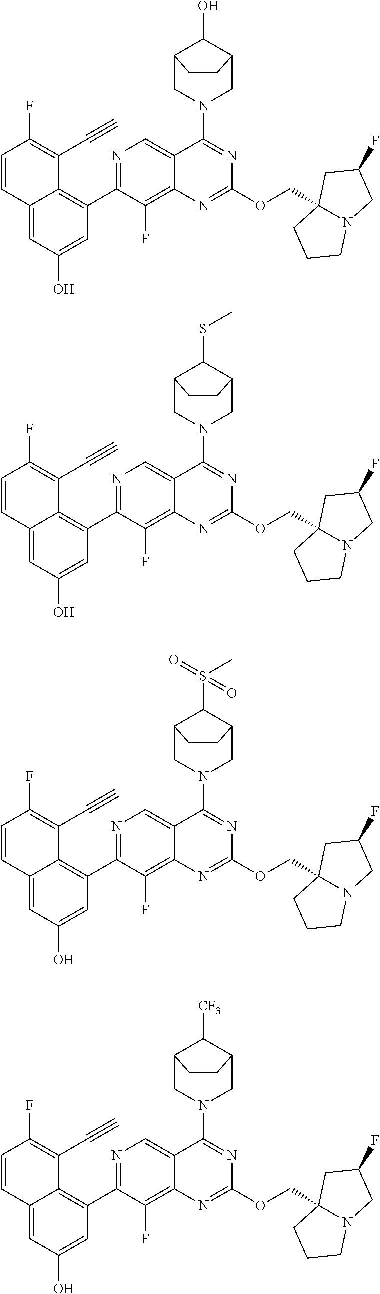 [SMILES: C#Cc1c(F)ccc2cc(O)cc(-c3ncc4c(N5CC6CCC(C5)C6O)nc(OC[C@@]56CCCN5C[C@H](F)C6)nc4c3F)c12 ; C#Cc1c(F)ccc2cc(O)cc(-c3ncc4c(N5CC6CCC(C5)C6SC)nc(OC[C@@]56CCCN5C[C@H](F)C6)nc4c3F)c12 ; C#Cc1c(F)ccc2cc(O)cc(-c3ncc4c(N5CC6CCC(C5)C6S(C)(=O)=O)nc(OC[C@@]56CCCN5C[C@H](F)C6)nc4c3F)c12 ; C#Cc1c(F)ccc2cc(O)cc(-c3ncc4c(N5CC6CCC(C5)C6C(F)(F)F)nc(OC[C@@]56CCCN5C[C@H](F)C6)nc4c3F)c12]

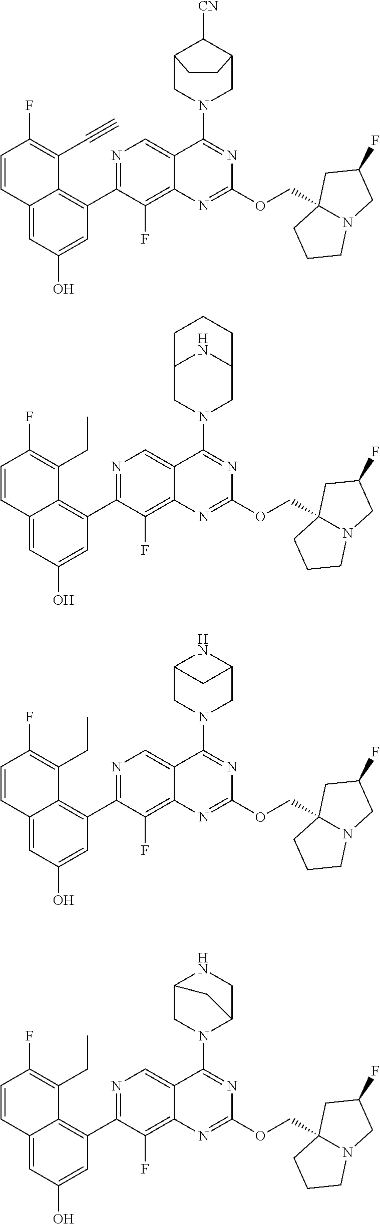 [SMILES: C#Cc1c(F)ccc2cc(O)cc(-c3ncc4c(N5CC6CCC(C5)C6C#N)nc(OC[C@@]56CCCN5C[C@H](F)C6)nc4c3F)c12 ; CCc1c(F)ccc2cc(O)cc(-c3ncc4c(N5CC6CCCC(C5)N6)nc(OC[C@@]56CCCN5C[C@H](F)C6)nc4c3F)c12 ; CCc1c(F)ccc2cc(O)cc(-c3ncc4c(N5CC6CC(C5)N6)nc(OC[C@@]56CCCN5C[C@H](F)C6)nc4c3F)c12 ; CCc1c(F)ccc2cc(O)cc(-c3ncc4c(N5CC6CC5CN6)nc(OC[C@@]56CCCN5C[C@H](F)C6)nc4c3F)c12]

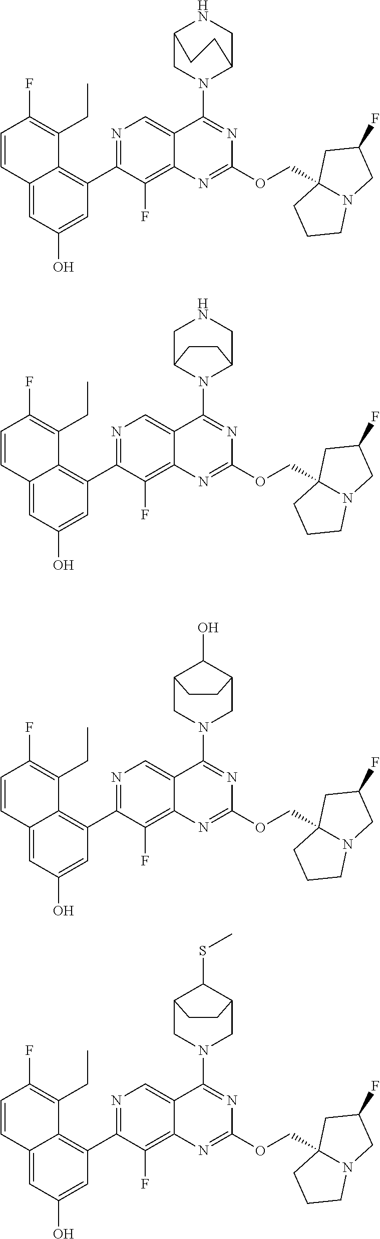 [SMILES: CCc1c(F)ccc2cc(O)cc(-c3ncc4c(N5CC6CCC5CN6)nc(OC[C@@]56CCCN5C[C@H](F)C6)nc4c3F)c12 ; CCc1c(F)ccc2cc(O)cc(-c3ncc4c(N5C6CCC5CNC6)nc(OC[C@@]56CCCN5C[C@H](F)C6)nc4c3F)c12 ; CCc1c(F)ccc2cc(O)cc(-c3ncc4c(N5CC6CCC(C5)C6O)nc(OC[C@@]56CCCN5C[C@H](F)C6)nc4c3F)c12 ; CCc1c(F)ccc2cc(O)cc(-c3ncc4c(N5CC6CCC(C5)C6SC)nc(OC[C@@]56CCCN5C[C@H](F)C6)nc4c3F)c12]

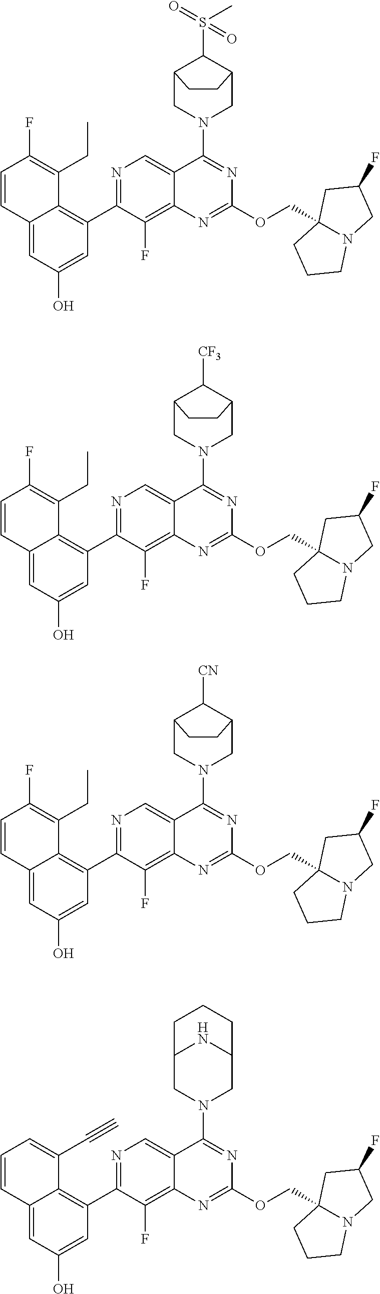 [SMILES: CCc1c(F)ccc2cc(O)cc(-c3ncc4c(N5CC6CCC(C5)C6S(C)(=O)=O)nc(OC[C@@]56CCCN5C[C@H](F)C6)nc4c3F)c12 ; CCc1c(F)ccc2cc(O)cc(-c3ncc4c(N5CC6CCC(C5)C6C(F)(F)F)nc(OC[C@@]56CCCN5C[C@H](F)C6)nc4c3F)c12 ; CCc1c(F)ccc2cc(O)cc(-c3ncc4c(N5CC6CCC(C5)C6C#N)nc(OC[C@@]56CCCN5C[C@H](F)C6)nc4c3F)c12 ; C#Cc1cccc2cc(O)cc(-c3ncc4c(N5CC6CCCC(C5)N6)nc(OC[C@@]56CCCN5C[C@H](F)C6)nc4c3F)c12]

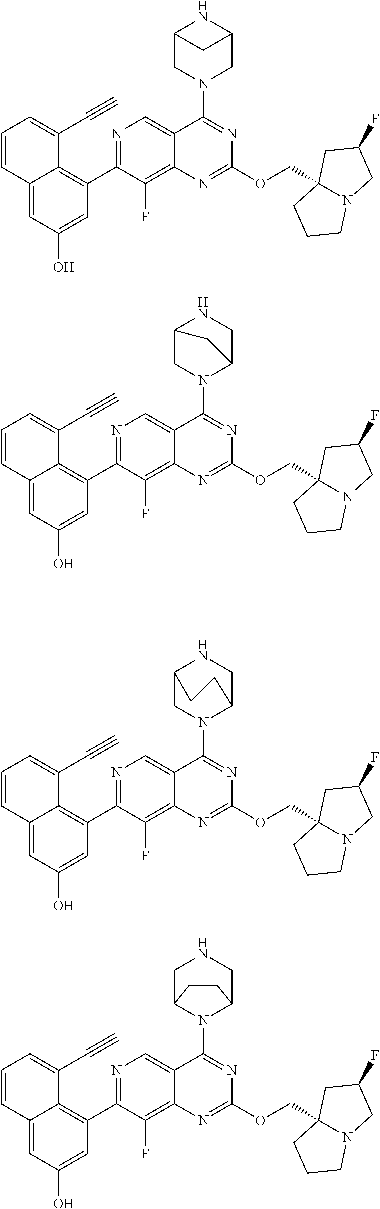 [SMILES: C#Cc1cccc2cc(O)cc(-c3ncc4c(N5CC6CC(C5)N6)nc(OC[C@@]56CCCN5C[C@H](F)C6)nc4c3F)c12 ; C#Cc1cccc2cc(O)cc(-c3ncc4c(N5CC6CC5CN6)nc(OC[C@@]56CCCN5C[C@H](F)C6)nc4c3F)c12 ; C#Cc1cccc2cc(O)cc(-c3ncc4c(N5CC6CCC5CN6)nc(OC[C@@]56CCCN5C[C@H](F)C6)nc4c3F)c12 ; C#Cc1cccc2cc(O)cc(-c3ncc4c(N5C6CCC5CNC6)nc(OC[C@@]56CCCN5C[C@H](F)C6)nc4c3F)c12]

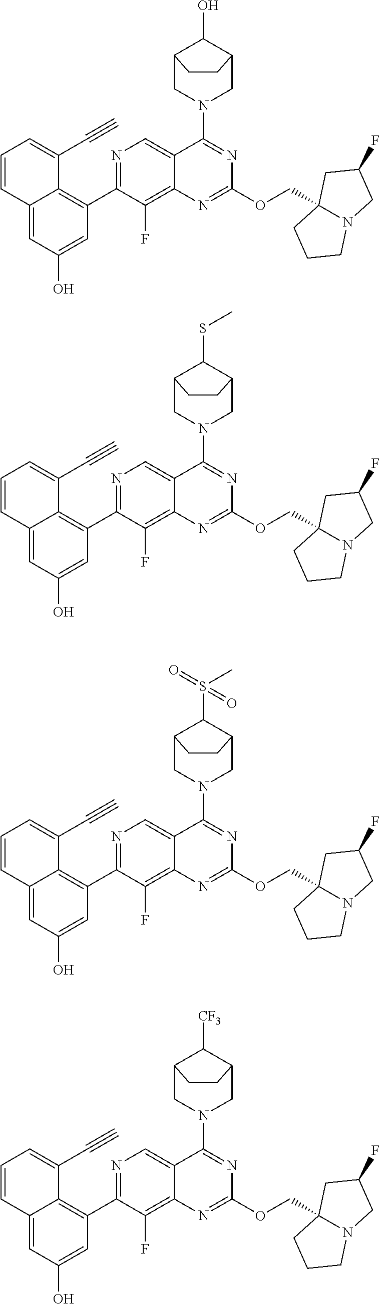 [SMILES: C#Cc1cccc2cc(O)cc(-c3ncc4c(N5CC6CCC(C5)C6O)nc(OC[C@@]56CCCN5C[C@H](F)C6)nc4c3F)c12 ; C#Cc1cccc2cc(O)cc(-c3ncc4c(N5CC6CCC(C5)C6SC)nc(OC[C@@]56CCCN5C[C@H](F)C6)nc4c3F)c12 ; C#Cc1cccc2cc(O)cc(-c3ncc4c(N5CC6CCC(C5)C6S(C)(=O)=O)nc(OC[C@@]56CCCN5C[C@H](F)C6)nc4c3F)c12 ; C#Cc1cccc2cc(O)cc(-c3ncc4c(N5CC6CCC(C5)C6C(F)(F)F)nc(OC[C@@]56CCCN5C[C@H](F)C6)nc4c3F)c12]

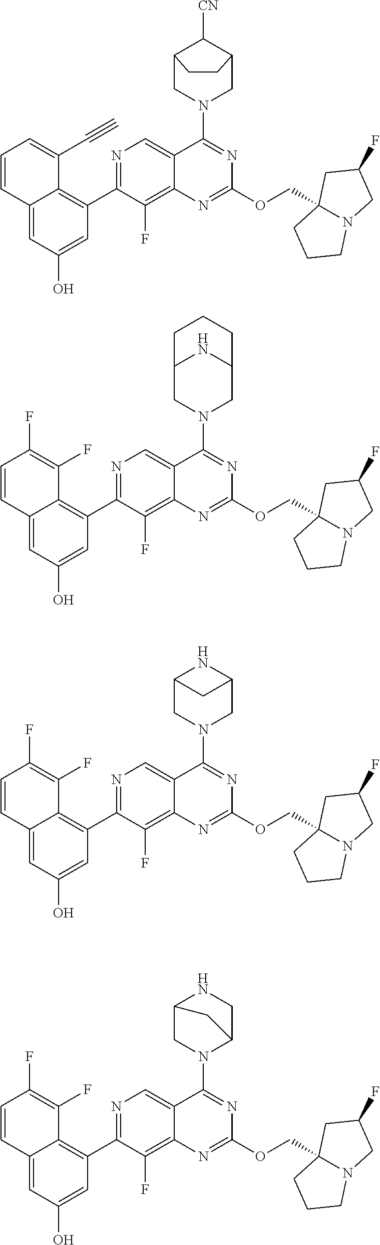 [SMILES: C#Cc1cccc2cc(O)cc(-c3ncc4c(N5CC6CCC(C5)C6C#N)nc(OC[C@@]56CCCN5C[C@H](F)C6)nc4c3F)c12 ; Oc1cc(-c2ncc3c(N4CC5CCCC(C4)N5)nc(OC[C@@]45CCCN4C[C@H](F)C5)nc3c2F)c2c(F)c(F)ccc2c1 ; Oc1cc(-c2ncc3c(N4CC5CC(C4)N5)nc(OC[C@@]45CCCN4C[C@H](F)C5)nc3c2F)c2c(F)c(F)ccc2c1 ; Oc1cc(-c2ncc3c(N4CC5CC4CN5)nc(OC[C@@]45CCCN4C[C@H](F)C5)nc3c2F)c2c(F)c(F)ccc2c1]

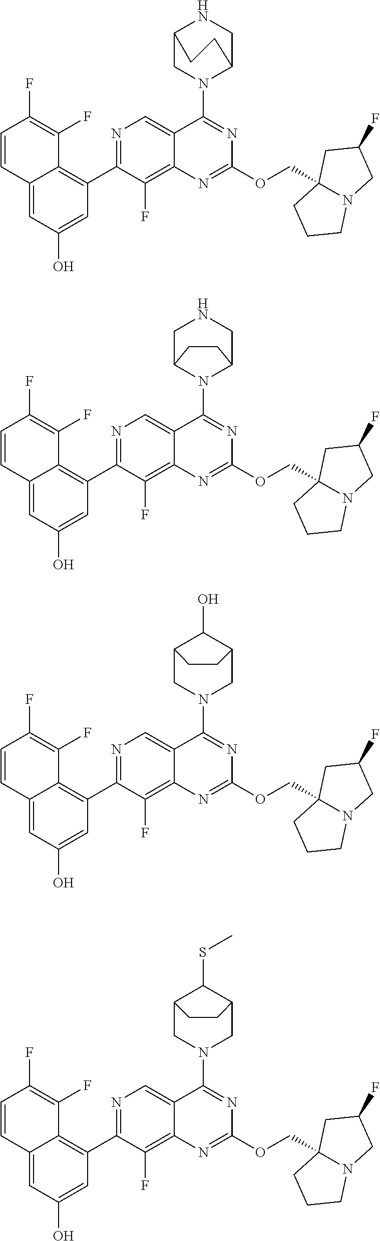 [SMILES: Oc1cc(-c2ncc3c(N4CC5CCC4CN5)nc(OC[C@@]45CCCN4C[C@H](F)C5)nc3c2F)c2c(F)c(F)ccc2c1 ; Oc1cc(-c2ncc3c(N4C5CCC4CNC5)nc(OC[C@@]45CCCN4C[C@H](F)C5)nc3c2F)c2c(F)c(F)ccc2c1 ; Oc1cc(-c2ncc3c(N4CC5CCC(C4)C5O)nc(OC[C@@]45CCCN4C[C@H](F)C5)nc3c2F)c2c(F)c(F)ccc2c1 ; CSC1C2CCC1CN(c1nc(OC[C@@]34CCCN3C[C@H](F)C4)nc3c(F)c(-c4cc(O)cc5ccc(F)c(F)c45)ncc13)C2]

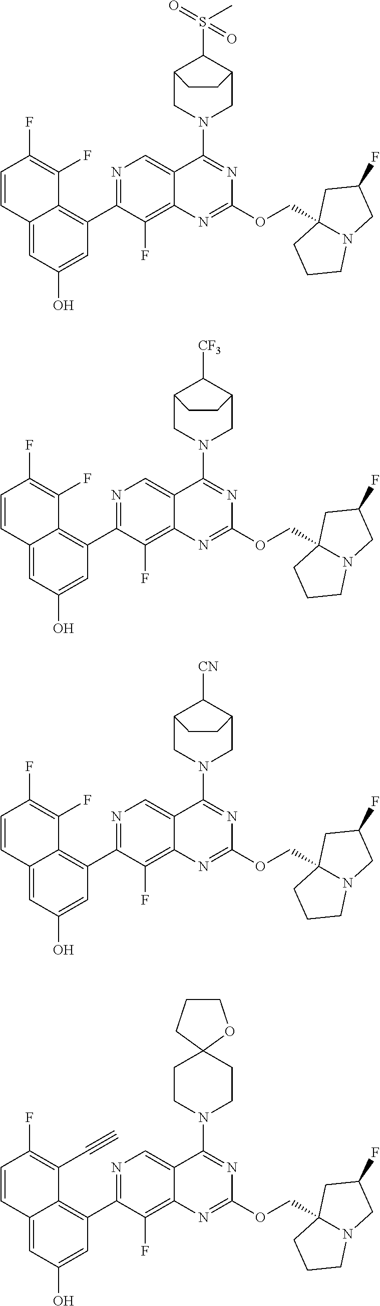 [SMILES: CS(=O)(=O)C1C2CCC1CN(c1nc(OC[C@@]34CCCN3C[C@H](F)C4)nc3c(F)c(-c4cc(O)cc5ccc(F)c(F)c45)ncc13)C2 ; Oc1cc(-c2ncc3c(N4CC5CCC(C4)C5C(F)(F)F)nc(OC[C@@]45CCCN4C[C@H](F)C5)nc3c2F)c2c(F)c(F)ccc2c1 ; N#CC1C2CCC1CN(c1nc(OC[C@@]34CCCN3C[C@H](F)C4)nc3c(F)c(-c4cc(O)cc5ccc(F)c(F)c45)ncc13)C2 ; C#Cc1c(F)ccc2cc(O)cc(-c3ncc4c(N5CCC6(CCCO6)CC5)nc(OC[C@@]56CCCN5C[C@H](F)C6)nc4c3F)c12]

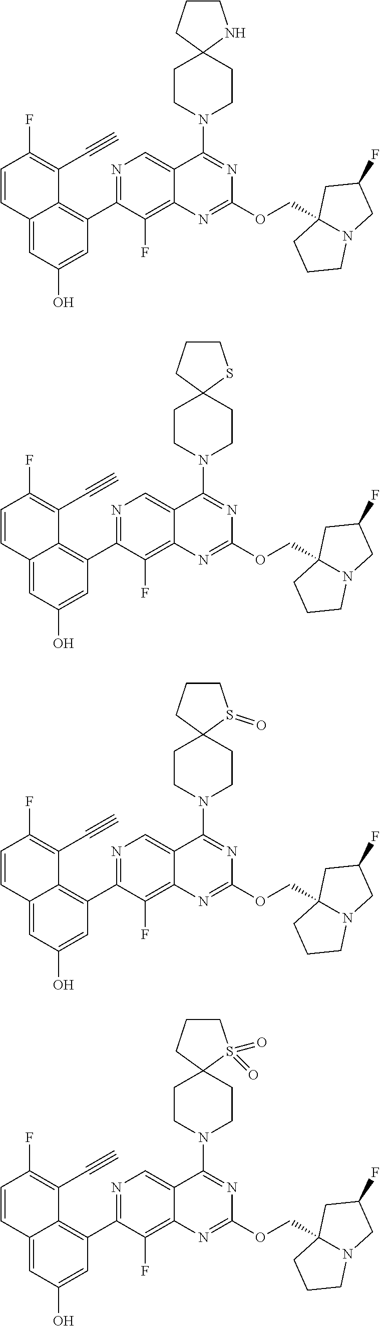 [SMILES: C#Cc1c(F)ccc2cc(O)cc(-c3ncc4c(N5CCC6(CCCN6)CC5)nc(OC[C@@]56CCCN5C[C@H](F)C6)nc4c3F)c12 ; C#Cc1c(F)ccc2cc(O)cc(-c3ncc4c(N5CCC6(CCCS6)CC5)nc(OC[C@@]56CCCN5C[C@H](F)C6)nc4c3F)c12 ; C#Cc1c(F)ccc2cc(O)cc(-c3ncc4c(N5CCC6(CCCS6=O)CC5)nc(OC[C@@]56CCCN5C[C@H](F)C6)nc4c3F)c12 ; C#Cc1c(F)ccc2cc(O)cc(-c3ncc4c(N5CCC6(CCCS6(=O)=O)CC5)nc(OC[C@@]56CCCN5C[C@H](F)C6)nc4c3F)c12]

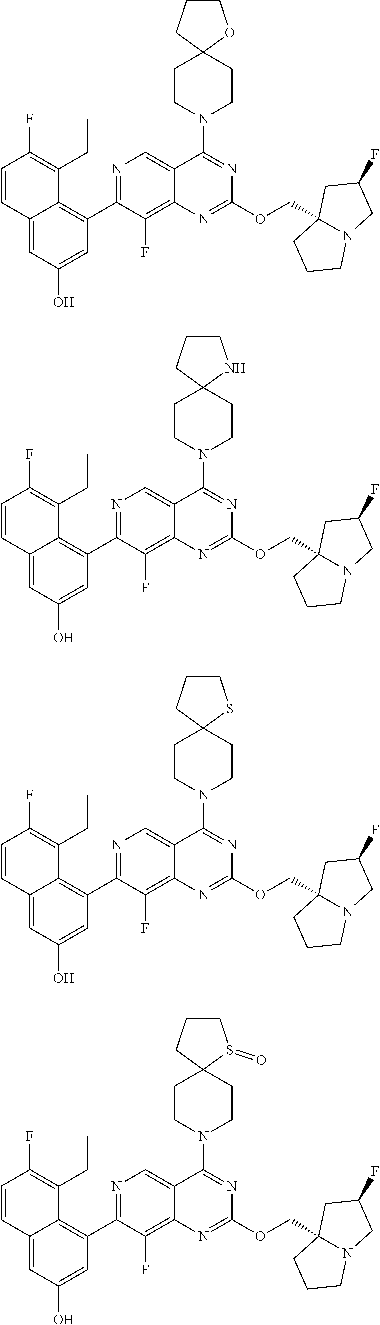 [SMILES: CCc1c(F)ccc2cc(O)cc(-c3ncc4c(N5CCC6(CCCO6)CC5)nc(OC[C@@]56CCCN5C[C@H](F)C6)nc4c3F)c12 ; CCc1c(F)ccc2cc(O)cc(-c3ncc4c(N5CCC6(CCCN6)CC5)nc(OC[C@@]56CCCN5C[C@H](F)C6)nc4c3F)c12 ; CCc1c(F)ccc2cc(O)cc(-c3ncc4c(N5CCC6(CCCS6)CC5)nc(OC[C@@]56CCCN5C[C@H](F)C6)nc4c3F)c12 ; CCc1c(F)ccc2cc(O)cc(-c3ncc4c(N5CCC6(CCCS6=O)CC5)nc(OC[C@@]56CCCN5C[C@H](F)C6)nc4c3F)c12]

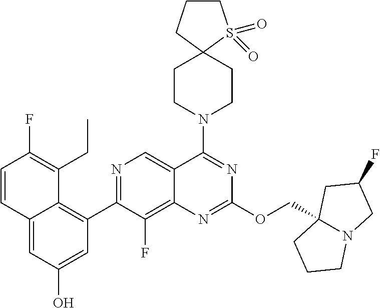 [SMILES: CCc1c(F)ccc2cc(O)cc(-c3ncc4c(N5CCC6(CCCS6(=O)=O)CC5)nc(OC[C@@]56CCCN5C[C@H](F)C6)nc4c3F)c12]

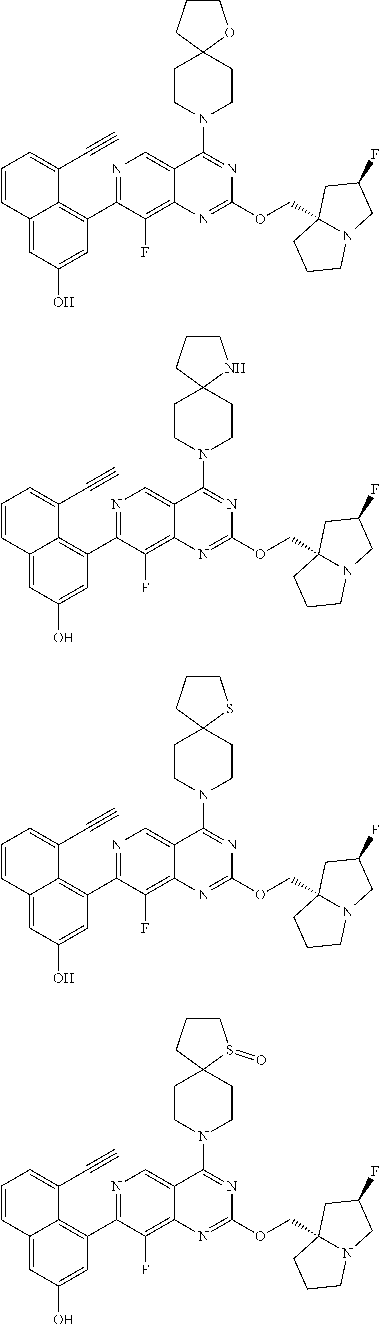 [SMILES: C#Cc1cccc2cc(O)cc(-c3ncc4c(N5CCC6(CCCO6)CC5)nc(OC[C@@]56CCCN5C[C@H](F)C6)nc4c3F)c12 ; C#Cc1cccc2cc(O)cc(-c3ncc4c(N5CCC6(CCCN6)CC5)nc(OC[C@@]56CCCN5C[C@H](F)C6)nc4c3F)c12 ; C#Cc1cccc2cc(O)cc(-c3ncc4c(N5CCC6(CCCS6)CC5)nc(OC[C@@]56CCCN5C[C@H](F)C6)nc4c3F)c12 ; C#Cc1cccc2cc(O)cc(-c3ncc4c(N5CCC6(CCCS6=O)CC5)nc(OC[C@@]56CCCN5C[C@H](F)C6)nc4c3F)c12]

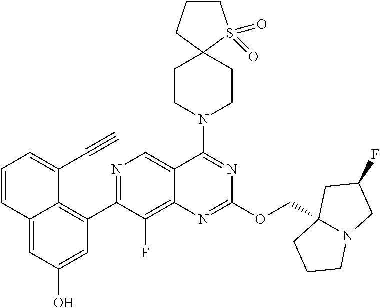 [SMILES: C#Cc1cccc2cc(O)cc(-c3ncc4c(N5CCC6(CCCS6(=O)=O)CC5)nc(OC[C@@]56CCCN5C[C@H](F)C6)nc4c3F)c12]

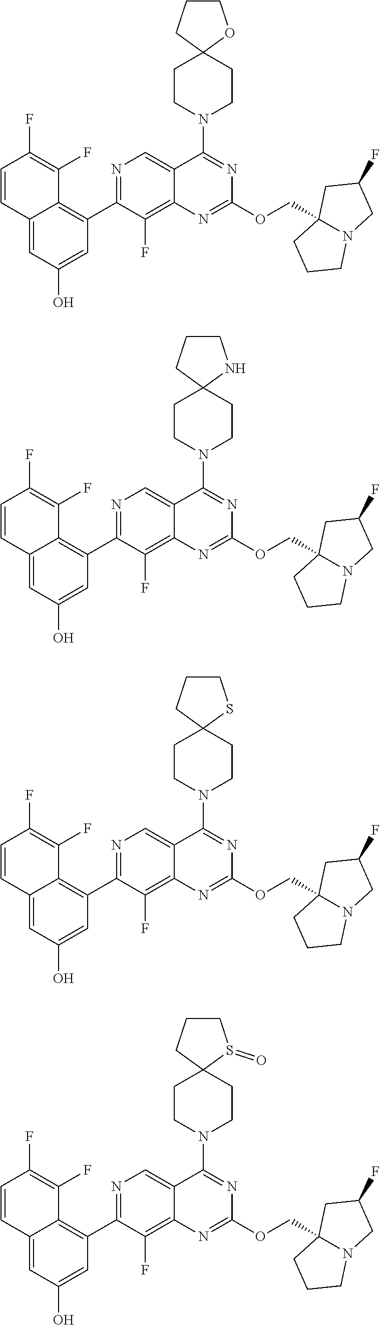 [SMILES: Oc1cc(-c2ncc3c(N4CCC5(CCCO5)CC4)nc(OC[C@@]45CCCN4C[C@H](F)C5)nc3c2F)c2c(F)c(F)ccc2c1 ; Oc1cc(-c2ncc3c(N4CCC5(CCCN5)CC4)nc(OC[C@@]45CCCN4C[C@H](F)C5)nc3c2F)c2c(F)c(F)ccc2c1 ; Oc1cc(-c2ncc3c(N4CCC5(CCCS5)CC4)nc(OC[C@@]45CCCN4C[C@H](F)C5)nc3c2F)c2c(F)c(F)ccc2c1 ; O=S1CCCC12CCN(c1nc(OC[C@@]34CCCN3C[C@H](F)C4)nc3c(F)c(-c4cc(O)cc5ccc(F)c(F)c45)ncc13)CC2]

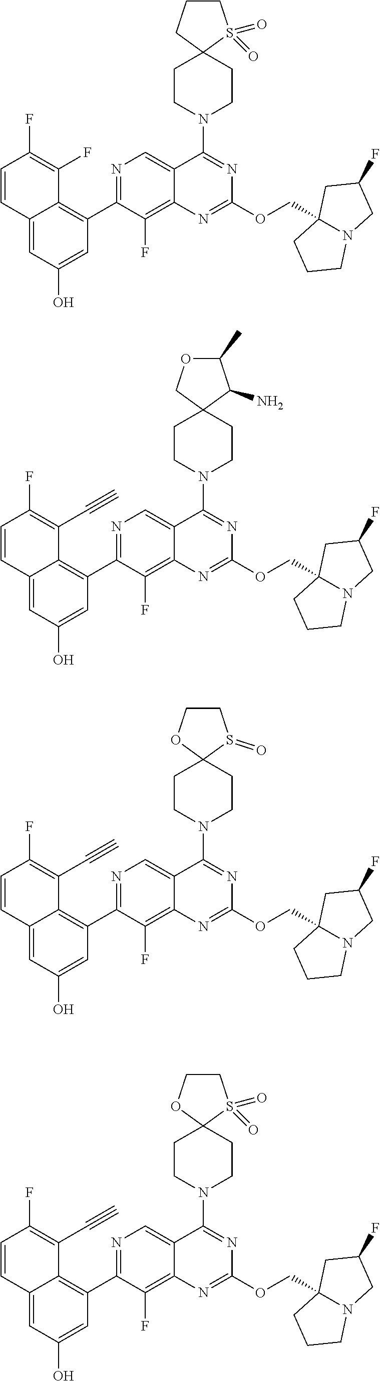 [SMILES: O=S1(=O)CCCC12CCN(c1nc(OC[C@@]34CCCN3C[C@H](F)C4)nc3c(F)c(-c4cc(O)cc5ccc(F)c(F)c45)ncc13)CC2 ; C#Cc1c(F)ccc2cc(O)cc(-c3ncc4c(N5CCC6(CC5)CO[C@@H](C)[C@H]6N)nc(OC[C@@]56CCCN5C[C@H](F)C6)nc4c3F)c12 ; C#Cc1c(F)ccc2cc(O)cc(-c3ncc4c(N5CCC6(CC5)OCCS6=O)nc(OC[C@@]56CCCN5C[C@H](F)C6)nc4c3F)c12 ; C#Cc1c(F)ccc2cc(O)cc(-c3ncc4c(N5CCC6(CC5)OCCS6(=O)=O)nc(OC[C@@]56CCCN5C[C@H](F)C6)nc4c3F)c12]

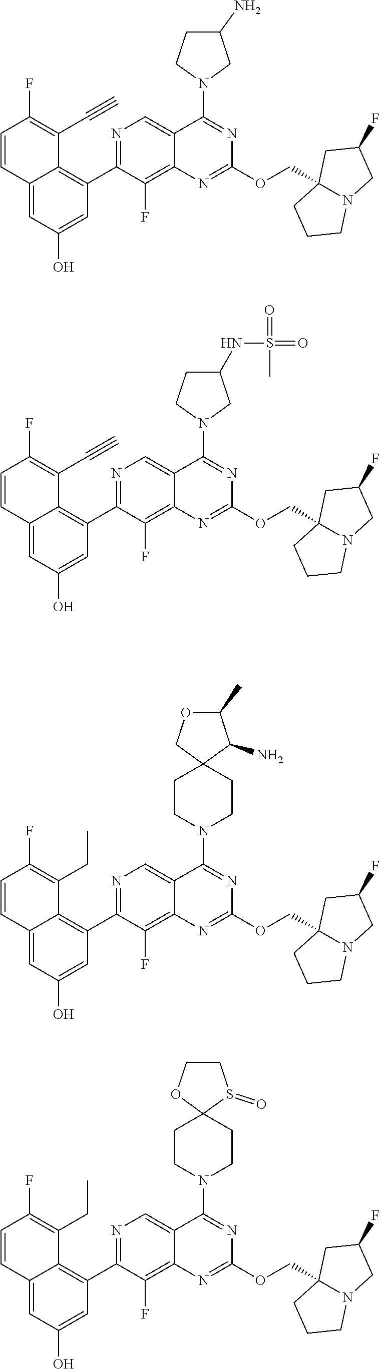 [SMILES: C#Cc1c(F)ccc2cc(O)cc(-c3ncc4c(N5CCC(N)C5)nc(OC[C@@]56CCCN5C[C@H](F)C6)nc4c3F)c12 ; C#Cc1c(F)ccc2cc(O)cc(-c3ncc4c(N5CCC(NS(C)(=O)=O)C5)nc(OC[C@@]56CCCN5C[C@H](F)C6)nc4c3F)c12 ; CCc1c(F)ccc2cc(O)cc(-c3ncc4c(N5CCC6(CC5)CO[C@@H](C)[C@H]6N)nc(OC[C@@]56CCCN5C[C@H](F)C6)nc4c3F)c12 ; CCc1c(F)ccc2cc(O)cc(-c3ncc4c(N5CCC6(CC5)OCCS6=O)nc(OC[C@@]56CCCN5C[C@H](F)C6)nc4c3F)c12]

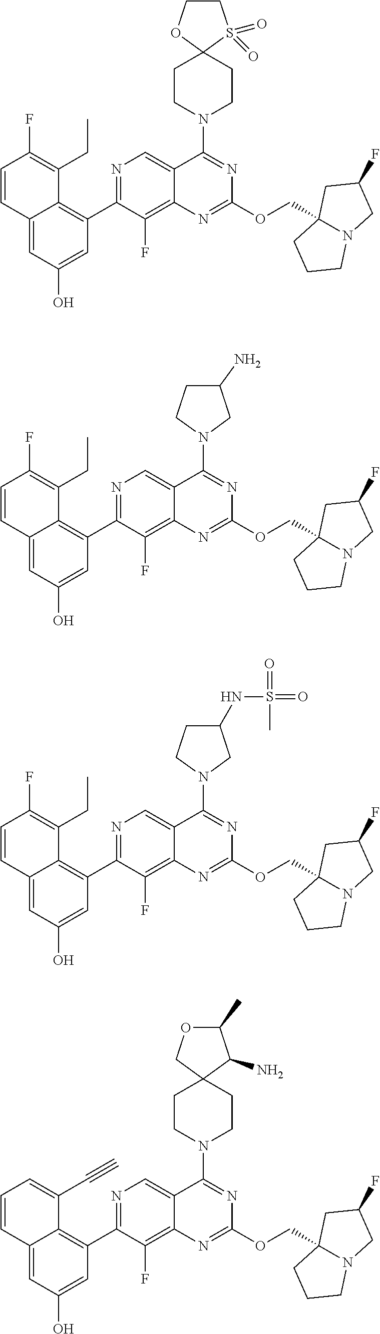 [SMILES: CCc1c(F)ccc2cc(O)cc(-c3ncc4c(N5CCC6(CC5)OCCS6(=O)=O)nc(OC[C@@]56CCCN5C[C@H](F)C6)nc4c3F)c12 ; CCc1c(F)ccc2cc(O)cc(-c3ncc4c(N5CCC(N)C5)nc(OC[C@@]56CCCN5C[C@H](F)C6)nc4c3F)c12 ; CCc1c(F)ccc2cc(O)cc(-c3ncc4c(N5CCC(NS(C)(=O)=O)C5)nc(OC[C@@]56CCCN5C[C@H](F)C6)nc4c3F)c12 ; C#Cc1cccc2cc(O)cc(-c3ncc4c(N5CCC6(CC5)CO[C@@H](C)[C@H]6N)nc(OC[C@@]56CCCN5C[C@H](F)C6)nc4c3F)c12]

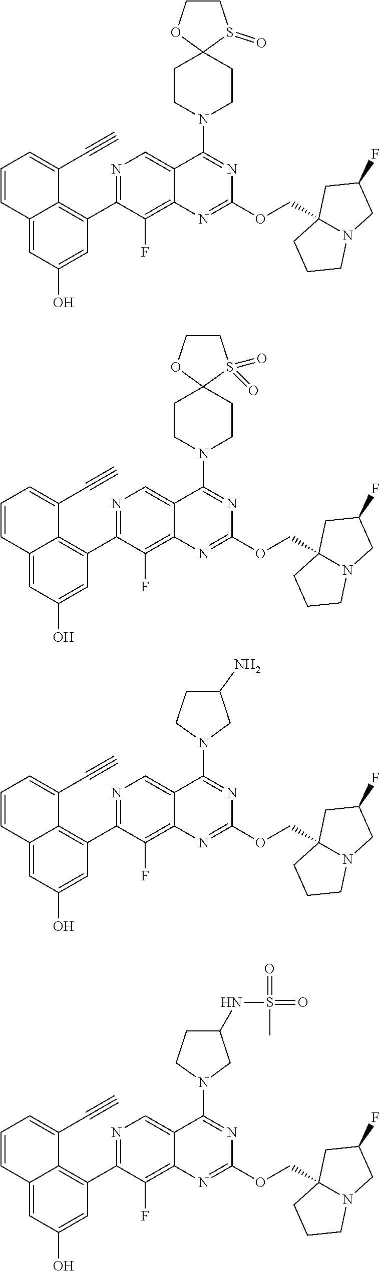 [SMILES: C#Cc1cccc2cc(O)cc(-c3ncc4c(N5CCC6(CC5)OCCS6=O)nc(OC[C@@]56CCCN5C[C@H](F)C6)nc4c3F)c12 ; C#Cc1cccc2cc(O)cc(-c3ncc4c(N5CCC6(CC5)OCCS6(=O)=O)nc(OC[C@@]56CCCN5C[C@H](F)C6)nc4c3F)c12 ; C#Cc1cccc2cc(O)cc(-c3ncc4c(N5CCC(N)C5)nc(OC[C@@]56CCCN5C[C@H](F)C6)nc4c3F)c12 ; C#Cc1cccc2cc(O)cc(-c3ncc4c(N5CCC(NS(C)(=O)=O)C5)nc(OC[C@@]56CCCN5C[C@H](F)C6)nc4c3F)c12]

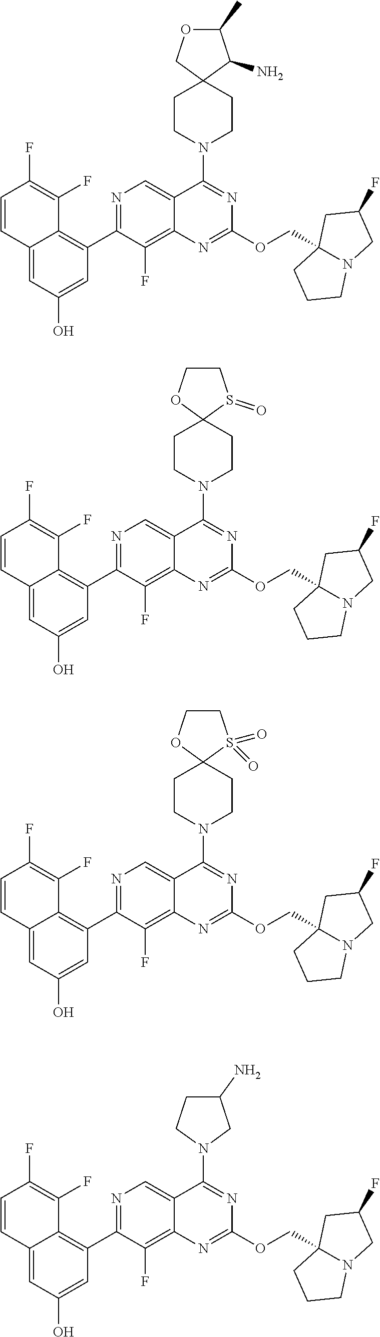 [SMILES: C[C@@H]1OCC2(CCN(c3nc(OC[C@@]45CCCN4C[C@H](F)C5)nc4c(F)c(-c5cc(O)cc6ccc(F)c(F)c56)ncc34)CC2)[C@@H]1N ; O=S1CCOC12CCN(c1nc(OC[C@@]34CCCN3C[C@H](F)C4)nc3c(F)c(-c4cc(O)cc5ccc(F)c(F)c45)ncc13)CC2 ; O=S1(=O)CCOC12CCN(c1nc(OC[C@@]34CCCN3C[C@H](F)C4)nc3c(F)c(-c4cc(O)cc5ccc(F)c(F)c45)ncc13)CC2 ; NC1CCN(c2nc(OC[C@@]34CCCN3C[C@H](F)C4)nc3c(F)c(-c4cc(O)cc5ccc(F)c(F)c45)ncc23)C1]

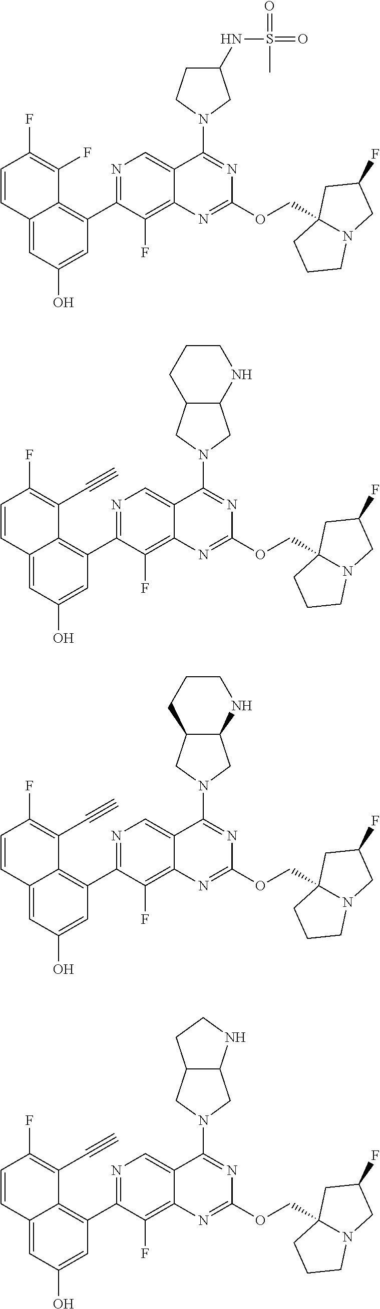 [SMILES: CS(=O)(=O)NC1CCN(c2nc(OC[C@@]34CCCN3C[C@H](F)C4)nc3c(F)c(-c4cc(O)cc5ccc(F)c(F)c45)ncc23)C1 ; C#Cc1c(F)ccc2cc(O)cc(-c3ncc4c(N5CC6CCCNC6C5)nc(OC[C@@]56CCCN5C[C@H](F)C6)nc4c3F)c12 ; C#Cc1c(F)ccc2cc(O)cc(-c3ncc4c(N5C[C@H]6CCCN[C@H]6C5)nc(OC[C@@]56CCCN5C[C@H](F)C6)nc4c3F)c12 ; C#Cc1c(F)ccc2cc(O)cc(-c3ncc4c(N5CC6CCNC6C5)nc(OC[C@@]56CCCN5C[C@H](F)C6)nc4c3F)c12]

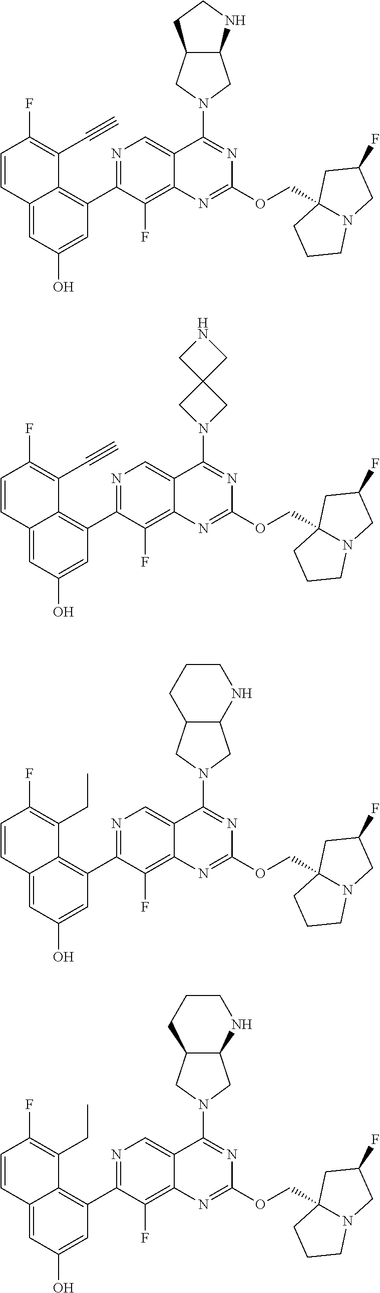 [SMILES: C#Cc1c(F)ccc2cc(O)cc(-c3ncc4c(N5C[C@H]6CCN[C@H]6C5)nc(OC[C@@]56CCCN5C[C@H](F)C6)nc4c3F)c12 ; C#Cc1c(F)ccc2cc(O)cc(-c3ncc4c(N5CC6(CNC6)C5)nc(OC[C@@]56CCCN5C[C@H](F)C6)nc4c3F)c12 ; CCc1c(F)ccc2cc(O)cc(-c3ncc4c(N5CC6CCCNC6C5)nc(OC[C@@]56CCCN5C[C@H](F)C6)nc4c3F)c12 ; CCc1c(F)ccc2cc(O)cc(-c3ncc4c(N5C[C@H]6CCCN[C@H]6C5)nc(OC[C@@]56CCCN5C[C@H](F)C6)nc4c3F)c12]

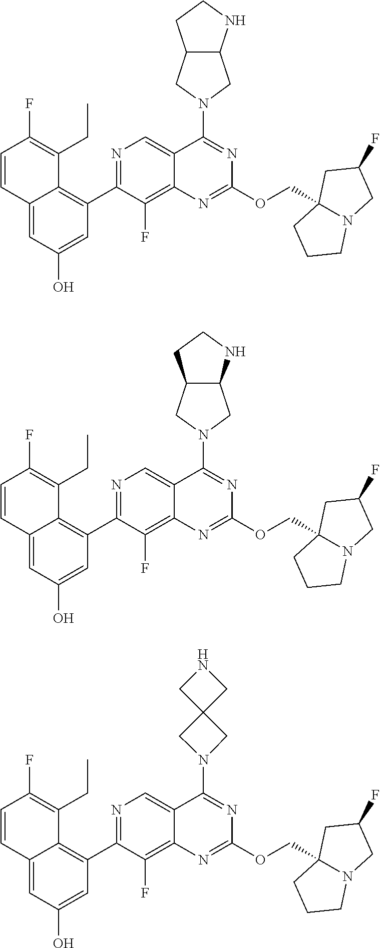 [SMILES: CCc1c(F)ccc2cc(O)cc(-c3ncc4c(N5CC6CCNC6C5)nc(OC[C@@]56CCCN5C[C@H](F)C6)nc4c3F)c12 ; CCc1c(F)ccc2cc(O)cc(-c3ncc4c(N5C[C@H]6CCN[C@H]6C5)nc(OC[C@@]56CCCN5C[C@H](F)C6)nc4c3F)c12 ; CCc1c(F)ccc2cc(O)cc(-c3ncc4c(N5CC6(CNC6)C5)nc(OC[C@@]56CCCN5C[C@H](F)C6)nc4c3F)c12]

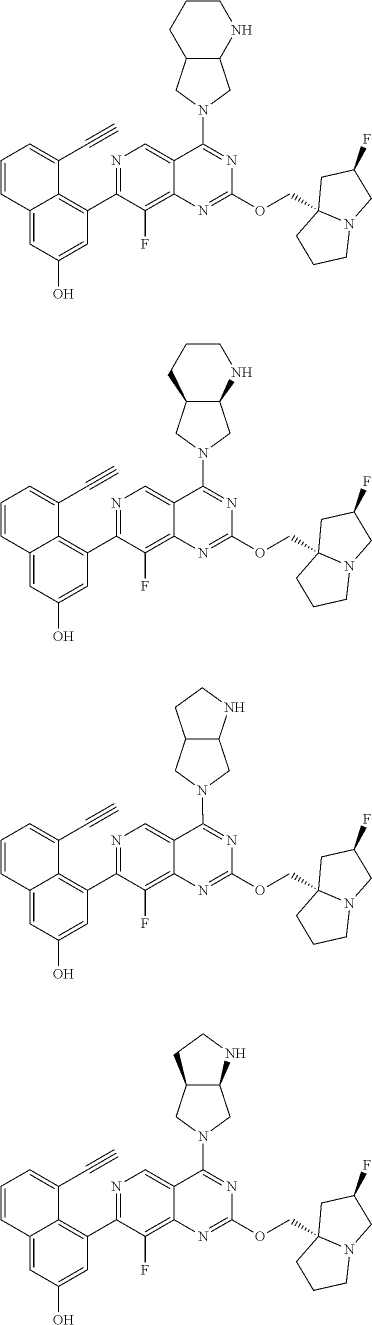 [SMILES: C#Cc1cccc2cc(O)cc(-c3ncc4c(N5CC6CCCNC6C5)nc(OC[C@@]56CCCN5C[C@H](F)C6)nc4c3F)c12 ; C#Cc1cccc2cc(O)cc(-c3ncc4c(N5C[C@H]6CCCN[C@H]6C5)nc(OC[C@@]56CCCN5C[C@H](F)C6)nc4c3F)c12 ; C#Cc1cccc2cc(O)cc(-c3ncc4c(N5CC6CCNC6C5)nc(OC[C@@]56CCCN5C[C@H](F)C6)nc4c3F)c12 ; C#Cc1cccc2cc(O)cc(-c3ncc4c(N5C[C@H]6CCN[C@H]6C5)nc(OC[C@@]56CCCN5C[C@H](F)C6)nc4c3F)c12]

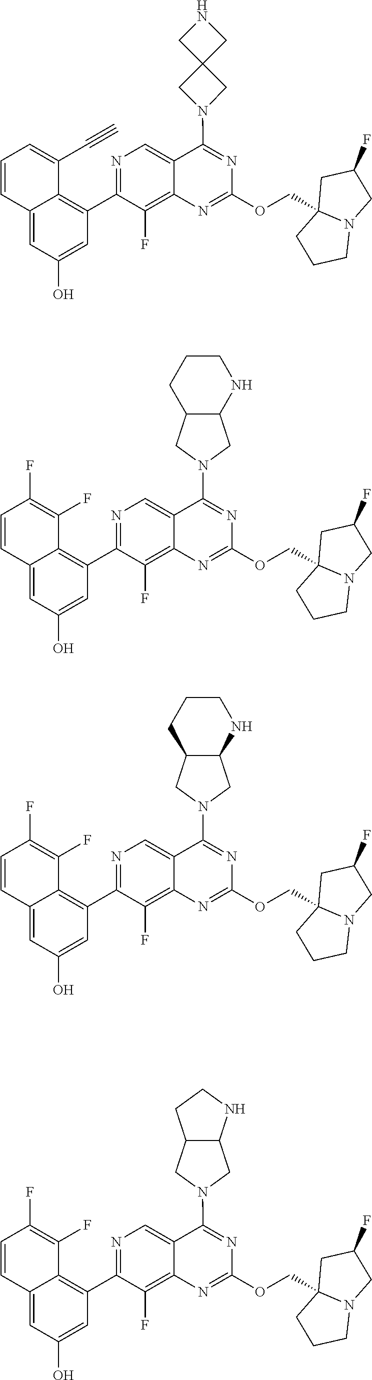 [SMILES: C#Cc1cccc2cc(O)cc(-c3ncc4c(N5CC6(CNC6)C5)nc(OC[C@@]56CCCN5C[C@H](F)C6)nc4c3F)c12 ; Oc1cc(-c2ncc3c(N4CC5CCCNC5C4)nc(OC[C@@]45CCCN4C[C@H](F)C5)nc3c2F)c2c(F)c(F)ccc2c1 ; Oc1cc(-c2ncc3c(N4C[C@H]5CCCN[C@H]5C4)nc(OC[C@@]45CCCN4C[C@H](F)C5)nc3c2F)c2c(F)c(F)ccc2c1 ; Oc1cc(-c2ncc3c(N4CC5CCNC5C4)nc(OC[C@@]45CCCN4C[C@H](F)C5)nc3c2F)c2c(F)c(F)ccc2c1]

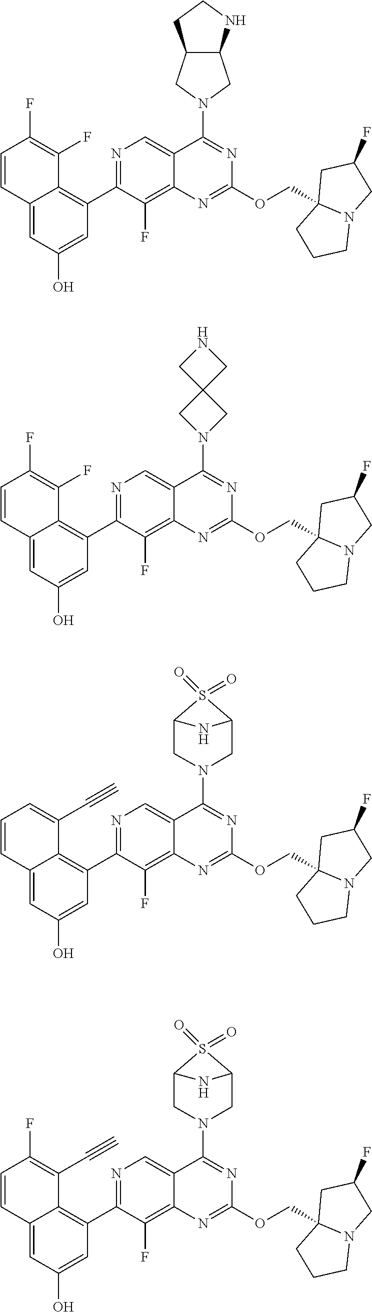 [SMILES: Oc1cc(-c2ncc3c(N4C[C@H]5CCN[C@H]5C4)nc(OC[C@@]45CCCN4C[C@H](F)C5)nc3c2F)c2c(F)c(F)ccc2c1 ; Oc1cc(-c2ncc3c(N4CC5(CNC5)C4)nc(OC[C@@]45CCCN4C[C@H](F)C5)nc3c2F)c2c(F)c(F)ccc2c1 ; C#Cc1cccc2cc(O)cc(-c3ncc4c(N5CC6NC(C5)S6(=O)=O)nc(OC[C@@]56CCCN5C[C@H](F)C6)nc4c3F)c12 ; C#Cc1c(F)ccc2cc(O)cc(-c3ncc4c(N5CC6NC(C5)S6(=O)=O)nc(OC[C@@]56CCCN5C[C@H](F)C6)nc4c3F)c12]

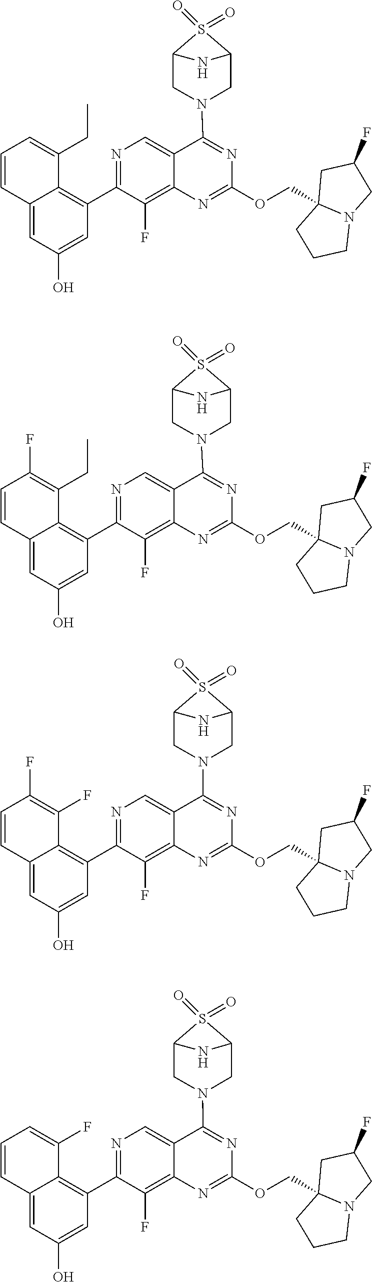 [SMILES: CCc1cccc2cc(O)cc(-c3ncc4c(N5CC6NC(C5)S6(=O)=O)nc(OC[C@@]56CCCN5C[C@H](F)C6)nc4c3F)c12 ; CCc1c(F)ccc2cc(O)cc(-c3ncc4c(N5CC6NC(C5)S6(=O)=O)nc(OC[C@@]56CCCN5C[C@H](F)C6)nc4c3F)c12 ; O=S1(=O)C2CN(c3nc(OC[C@@]45CCCN4C[C@H](F)C5)nc4c(F)c(-c5cc(O)cc6ccc(F)c(F)c56)ncc34)CC1N2 ; O=S1(=O)C2CN(c3nc(OC[C@@]45CCCN4C[C@H](F)C5)nc4c(F)c(-c5cc(O)cc6cccc(F)c56)ncc34)CC1N2]

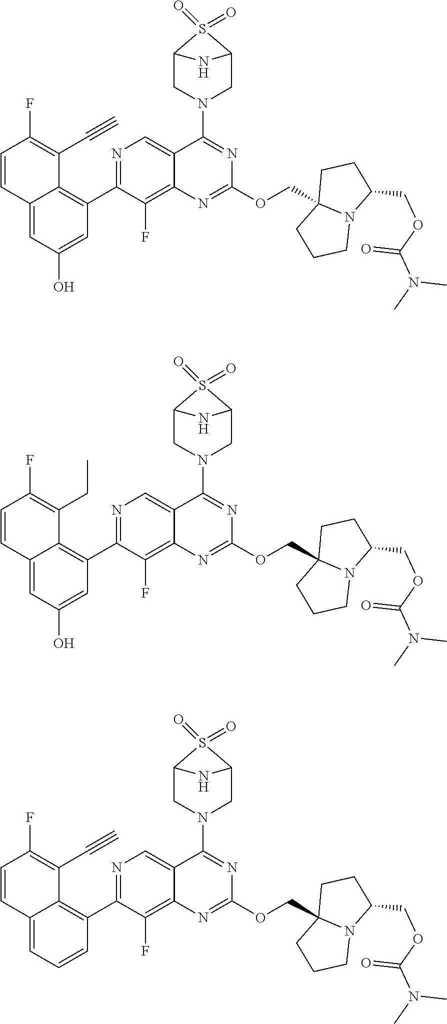 [SMILES: C#Cc1c(F)ccc2cc(O)cc(-c3ncc4c(N5CC6NC(C5)S6(=O)=O)nc(OC[C@@]56CCCN5[C@@H](COC(=O)N(C)C)CC6)nc4c3F)c12 ; CCc1c(F)ccc2cc(O)cc(-c3ncc4c(N5CC6NC(C5)S6(=O)=O)nc(OC[C@]56CCCN5[C@@H](COC(=O)N(C)C)CC6)nc4c3F)c12 ; C#Cc1c(F)ccc2cccc(-c3ncc4c(N5CC6NC(C5)S6(=O)=O)nc(OC[C@]56CCCN5[C@@H](COC(=O)N(C)C)CC6)nc4c3F)c12]

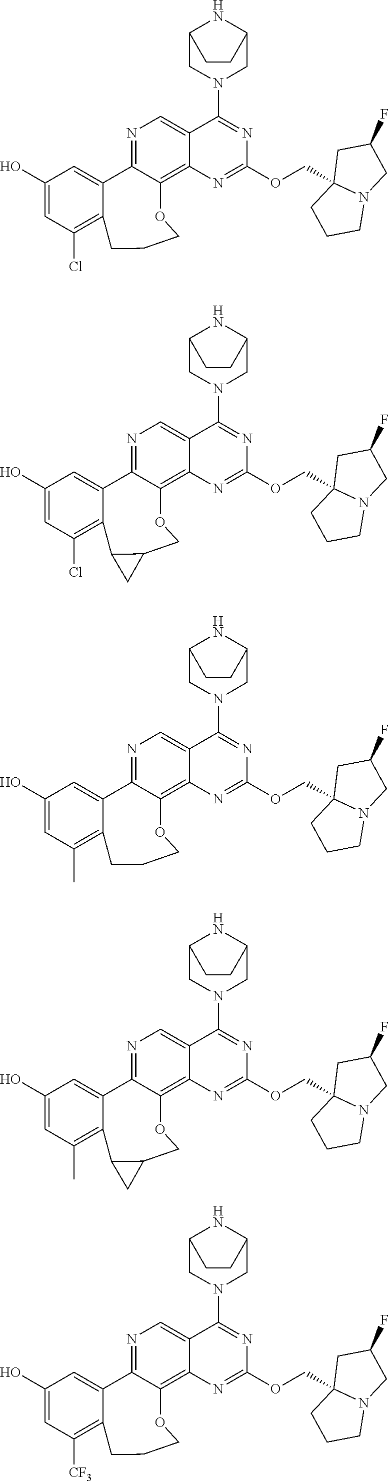 [SMILES: Oc1cc(Cl)c2c(c1)-c1ncc3c(N4CC5CCC(C4)N5)nc(OC[C@@]45CCCN4C[C@H](F)C5)nc3c1OCCC2 ; Oc1cc(Cl)c2c(c1)-c1ncc3c(N4CC5CCC(C4)N5)nc(OC[C@@]45CCCN4C[C@H](F)C5)nc3c1OCC1CC21 ; Cc1cc(O)cc2c1CCCOc1c-2ncc2c(N3CC4CCC(C3)N4)nc(OC[C@@]34CCCN3C[C@H](F)C4)nc12 ; Cc1cc(O)cc2c1C1CC1COc1c-2ncc2c(N3CC4CCC(C3)N4)nc(OC[C@@]34CCCN3C[C@H](F)C4)nc12 ; Oc1cc2c(c(C(F)(F)F)c1)CCCOc1c-2ncc2c(N3CC4CCC(C3)N4)nc(OC[C@@]34CCCN3C[C@H](F)C4)nc12]

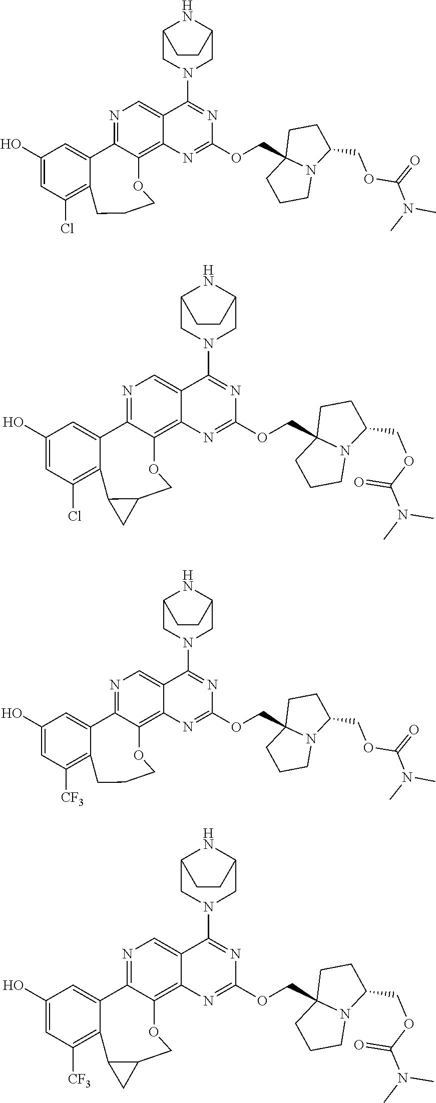 [SMILES: CN(C)C(=O)OC[C@H]1CC[C@@]2(COc3nc(N4CC5CCC(C4)N5)c4cnc5c(c4n3)OCCCc3c(Cl)cc(O)cc3-5)CCCN12 ; CN(C)C(=O)OC[C@H]1CC[C@@]2(COc3nc(N4CC5CCC(C4)N5)c4cnc5c(c4n3)OCC3CC3c3c(Cl)cc(O)cc3-5)CCCN12 ; CN(C)C(=O)OC[C@H]1CC[C@@]2(COc3nc(N4CC5CCC(C4)N5)c4cnc5c(c4n3)OCCCc3c-5cc(O)cc3C(F)(F)F)CCCN12 ; CN(C)C(=O)OC[C@H]1CC[C@@]2(COc3nc(N4CC5CCC(C4)N5)c4cnc5c(c4n3)OCC3CC3c3c-5cc(O)cc3C(F)(F)F)CCCN12]

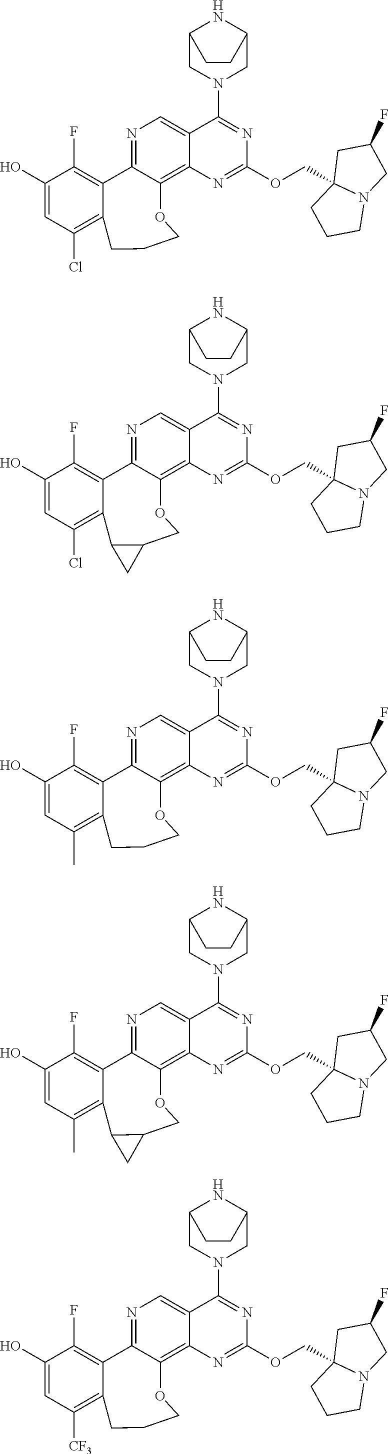 [SMILES: Oc1cc(Cl)c2c(c1F)-c1ncc3c(N4CC5CCC(C4)N5)nc(OC[C@@]45CCCN4C[C@H](F)C5)nc3c1OCCC2 ; Oc1cc(Cl)c2c(c1F)-c1ncc3c(N4CC5CCC(C4)N5)nc(OC[C@@]45CCCN4C[C@H](F)C5)nc3c1OCC1CC21 ; Cc1cc(O)c(F)c2c1CCCOc1c-2ncc2c(N3CC4CCC(C3)N4)nc(OC[C@@]34CCCN3C[C@H](F)C4)nc12 ; Cc1cc(O)c(F)c2c1C1CC1COc1c-2ncc2c(N3CC4CCC(C3)N4)nc(OC[C@@]34CCCN3C[C@H](F)C4)nc12 ; Oc1cc(C(F)(F)F)c2c(c1F)-c1ncc3c(N4CC5CCC(C4)N5)nc(OC[C@@]45CCCN4C[C@H](F)C5)nc3c1OCCC2]

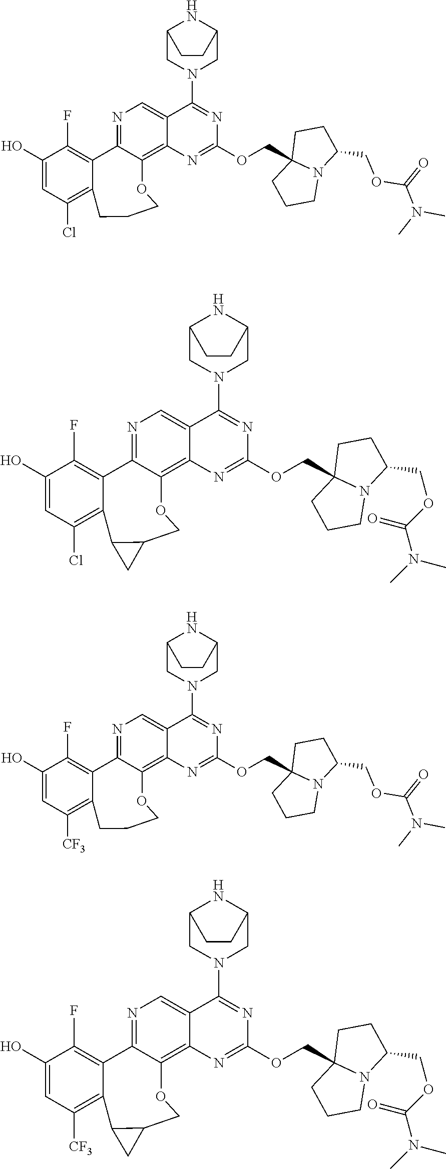 [SMILES: CN(C)C(=O)OC[C@H]1CC[C@@]2(COc3nc(N4CC5CCC(C4)N5)c4cnc5c(c4n3)OCCCc3c(Cl)cc(O)c(F)c3-5)CCCN12 ; CN(C)C(=O)OC[C@H]1CC[C@@]2(COc3nc(N4CC5CCC(C4)N5)c4cnc5c(c4n3)OCC3CC3c3c(Cl)cc(O)c(F)c3-5)CCCN12 ; CN(C)C(=O)OC[C@H]1CC[C@@]2(COc3nc(N4CC5CCC(C4)N5)c4cnc5c(c4n3)OCCCc3c(C(F)(F)F)cc(O)c(F)c3-5)CCCN12 ; CN(C)C(=O)OC[C@H]1CC[C@@]2(COc3nc(N4CC5CCC(C4)N5)c4cnc5c(c4n3)OCC3CC3c3c(C(F)(F)F)cc(O)c(F)c3-5)CCCN12]

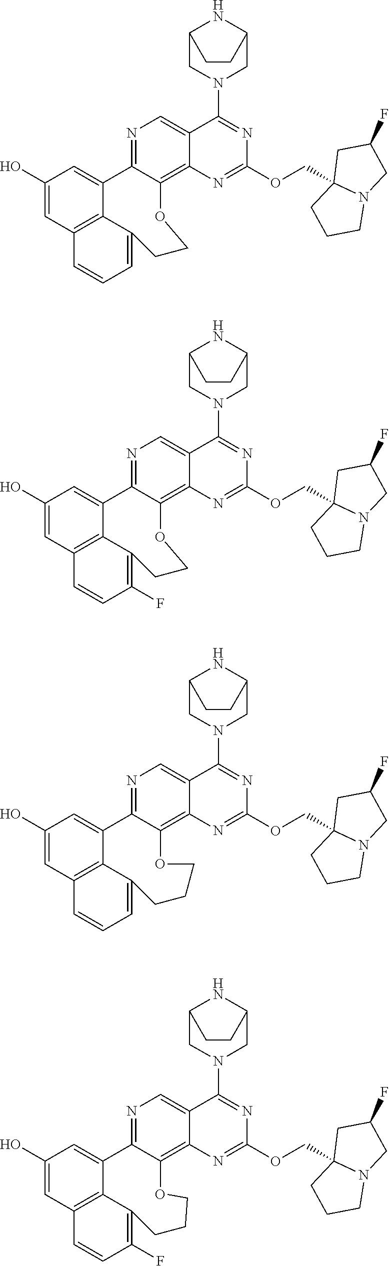 [SMILES: Oc1cc2c3c(cccc3c1)CCOc1c-2ncc2c(N3CC4CCC(C3)N4)nc(OC[C@@]34CCCN3C[C@H](F)C4)nc12 ; Oc1cc2c3c(c(F)ccc3c1)CCOc1c-2ncc2c(N3CC4CCC(C3)N4)nc(OC[C@@]34CCCN3C[C@H](F)C4)nc12 ; Oc1cc2c3c(cccc3c1)CCCOc1c-2ncc2c(N3CC4CCC(C3)N4)nc(OC[C@@]34CCCN3C[C@H](F)C4)nc12 ; Oc1cc2c3c(c(F)ccc3c1)CCCOc1c-2ncc2c(N3CC4CCC(C3)N4)nc(OC[C@@]34CCCN3C[C@H](F)C4)nc12]

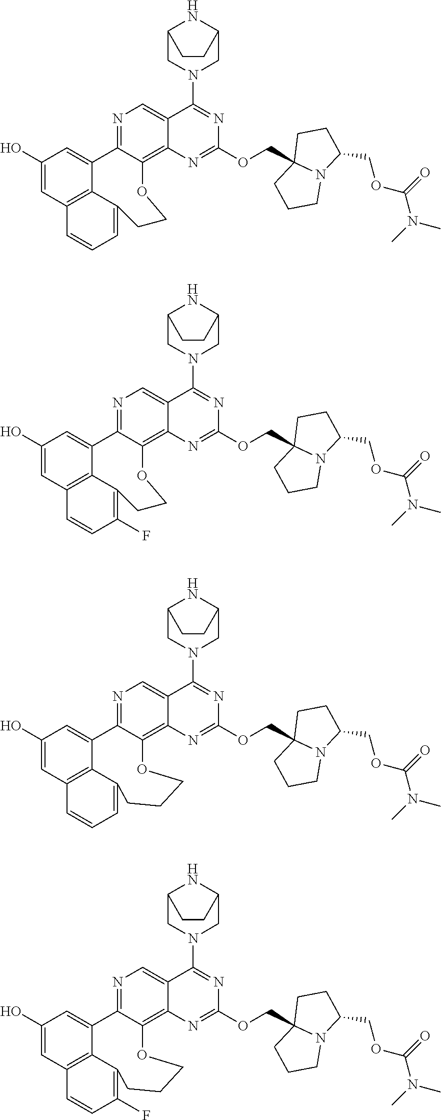 [SMILES: CN(C)C(=O)OC[C@H]1CC[C@@]2(COc3nc(N4CC5CCC(C4)N5)c4cnc5c(c4n3)OCCc3cccc4cc(O)cc-5c34)CCCN12 ; CN(C)C(=O)OC[C@H]1CC[C@@]2(COc3nc(N4CC5CCC(C4)N5)c4cnc5c(c4n3)OCCc3c(F)ccc4cc(O)cc-5c34)CCCN12 ; CN(C)C(=O)OC[C@H]1CC[C@@]2(COc3nc(N4CC5CCC(C4)N5)c4cnc5c(c4n3)OCCCc3cccc4cc(O)cc-5c34)CCCN12 ; CN(C)C(=O)OC[C@H]1CC[C@@]2(COc3nc(N4CC5CCC(C4)N5)c4cnc5c(c4n3)OCCCc3c(F)ccc4cc(O)cc-5c34)CCCN12]

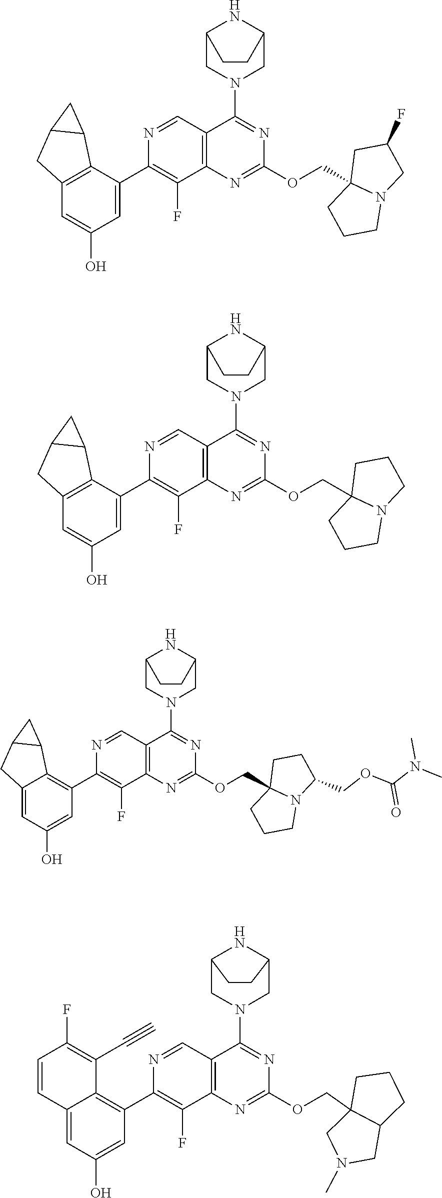 [SMILES: Oc1cc2c(c(-c3ncc4c(N5CC6CCC(C5)N6)nc(OC[C@@]56CCCN5C[C@H](F)C6)nc4c3F)c1)C1CC1C2 ; Oc1cc2c(c(-c3ncc4c(N5CC6CCC(C5)N6)nc(OCC56CCCN5CCC6)nc4c3F)c1)C1CC1C2 ; CN(C)C(=O)OC[C@H]1CC[C@@]2(COc3nc(N4CC5CCC(C4)N5)c4cnc(-c5cc(O)cc6c5C5CC5C6)c(F)c4n3)CCCN12 ; C#Cc1c(F)ccc2cc(O)cc(-c3ncc4c(N5CC6CCC(C5)N6)nc(OCC56CCCC5CN(C)C6)nc4c3F)c12]

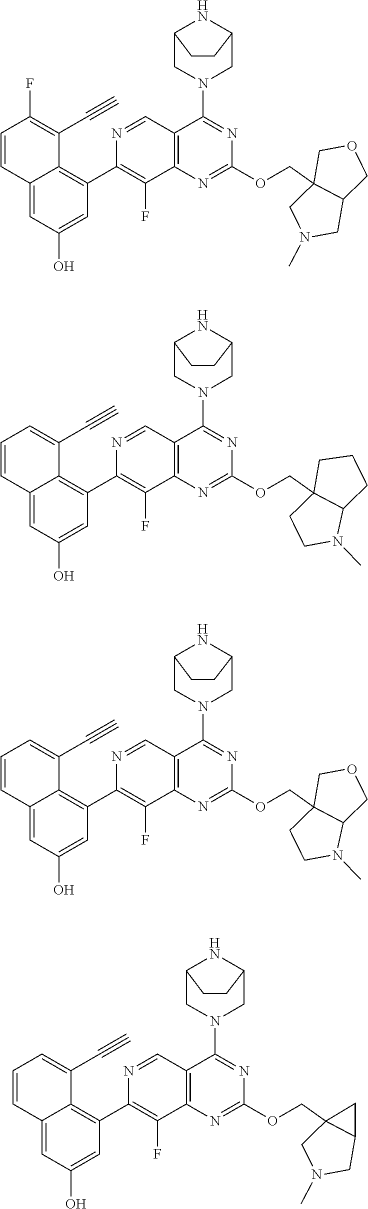 [SMILES: C#Cc1c(F)ccc2cc(O)cc(-c3ncc4c(N5CC6CCC(C5)N6)nc(OCC56COCC5CN(C)C6)nc4c3F)c12 ; C#Cc1cccc2cc(O)cc(-c3ncc4c(N5CC6CCC(C5)N6)nc(OCC56CCCC5N(C)CC6)nc4c3F)c12 ; C#Cc1cccc2cc(O)cc(-c3ncc4c(N5CC6CCC(C5)N6)nc(OCC56CCN(C)C5COC6)nc4c3F)c12 ; C#Cc1cccc2cc(O)cc(-c3ncc4c(N5CC6CCC(C5)N6)nc(OCC56CC5CN(C)C6)nc4c3F)c12]

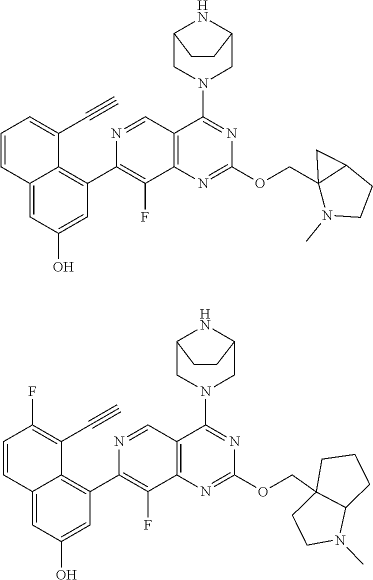 [SMILES: C#Cc1cccc2cc(O)cc(-c3ncc4c(N5CC6CCC(C5)N6)nc(OCC56CC5CCN6C)nc4c3F)c12 ; C#Cc1c(F)ccc2cc(O)cc(-c3ncc4c(N5CC6CCC(C5)N6)nc(OCC56CCCC5N(C)CC6)nc4c3F)c12]

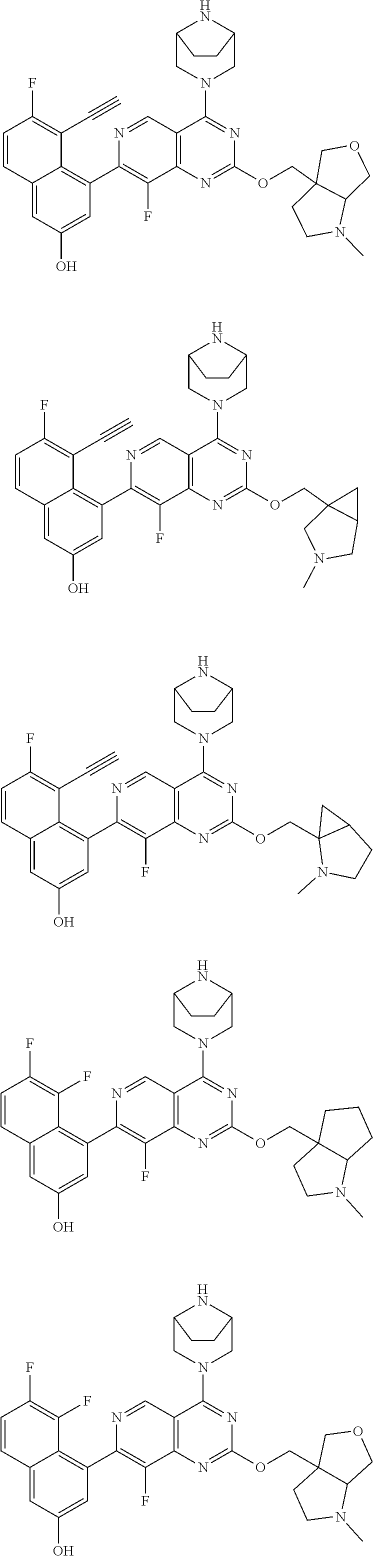 [SMILES: C#Cc1c(F)ccc2cc(O)cc(-c3ncc4c(N5CC6CCC(C5)N6)nc(OCC56CCN(C)C5COC6)nc4c3F)c12 ; C#Cc1c(F)ccc2cc(O)cc(-c3ncc4c(N5CC6CCC(C5)N6)nc(OCC56CC5CN(C)C6)nc4c3F)c12 ; C#Cc1c(F)ccc2cc(O)cc(-c3ncc4c(N5CC6CCC(C5)N6)nc(OCC56CC5CCN6C)nc4c3F)c12 ; CN1CCC2(COc3nc(N4CC5CCC(C4)N5)c4cnc(-c5cc(O)cc6ccc(F)c(F)c56)c(F)c4n3)CCCC12 ; CN1CCC2(COc3nc(N4CC5CCC(C4)N5)c4cnc(-c5cc(O)cc6ccc(F)c(F)c56)c(F)c4n3)COCC12]

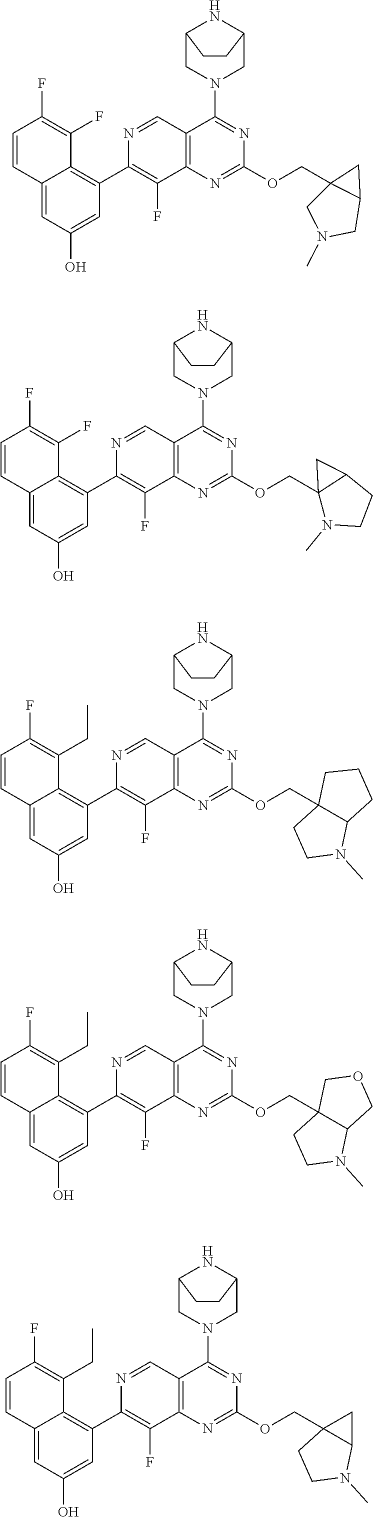 [SMILES: CN1CC2CC2(COc2nc(N3CC4CCC(C3)N4)c3cnc(-c4cc(O)cc5ccc(F)c(F)c45)c(F)c3n2)C1 ; CN1CCC2CC21COc1nc(N2CC3CCC(C2)N3)c2cnc(-c3cc(O)cc4ccc(F)c(F)c34)c(F)c2n1 ; CCc1c(F)ccc2cc(O)cc(-c3ncc4c(N5CC6CCC(C5)N6)nc(OCC56CCCC5N(C)CC6)nc4c3F)c12 ; CCc1c(F)ccc2cc(O)cc(-c3ncc4c(N5CC6CCC(C5)N6)nc(OCC56CCN(C)C5COC6)nc4c3F)c12 ; CCc1c(F)ccc2cc(O)cc(-c3ncc4c(N5CC6CCC(C5)N6)nc(OCC56CCN(C)C5C6)nc4c3F)c12]

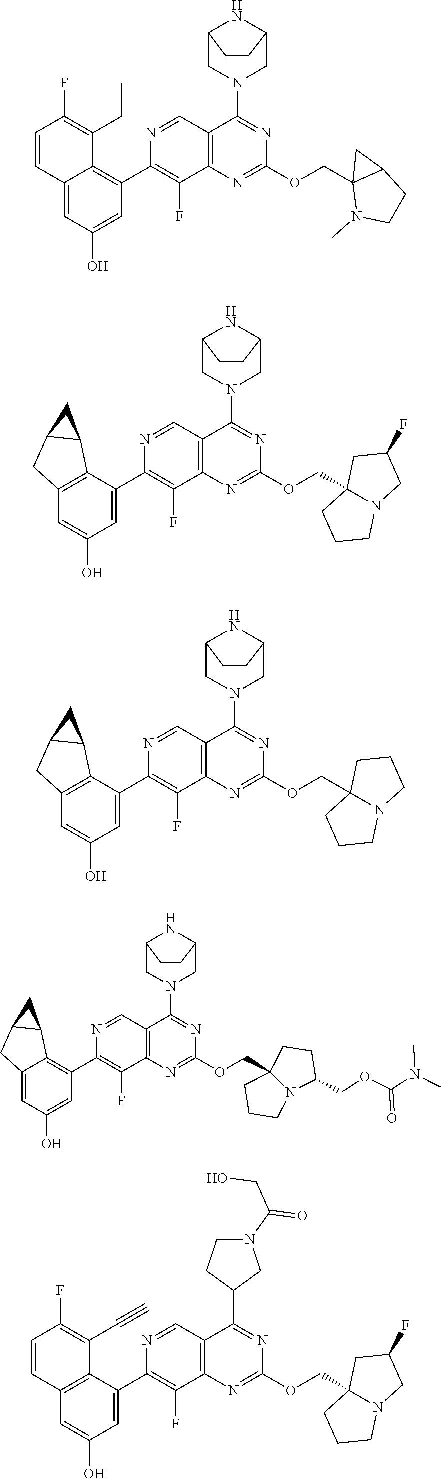 [SMILES: CCc1c(F)ccc2cc(O)cc(-c3ncc4c(N5CC6CCC(C5)N6)nc(OCC56CC5CCN6C)nc4c3F)c12 ; Oc1cc2c(c(-c3ncc4c(N5CC6CCC(C5)N6)nc(OC[C@@]56CCCN5C[C@H](F)C6)nc4c3F)c1)[C@@H]1C[C@@H]1C2 ; Oc1cc2c(c(-c3ncc4c(N5CC6CCC(C5)N6)nc(OCC56CCCN5CCC6)nc4c3F)c1)[C@@H]1C[C@@H]1C2 ; CN(C)C(=O)OC[C@H]1CC[C@@]2(COc3nc(N4CC5CCC(C4)N5)c4cnc(-c5cc(O)cc6c5[C@@H]5C[C@@H]5C6)c(F)c4n3)CCCN12 ; C#Cc1c(F)ccc2cc(O)cc(-c3ncc4c(C5CCN(C(=O)CO)C5)nc(OC[C@@]56CCCN5C[C@H](F)C6)nc4c3F)c12]

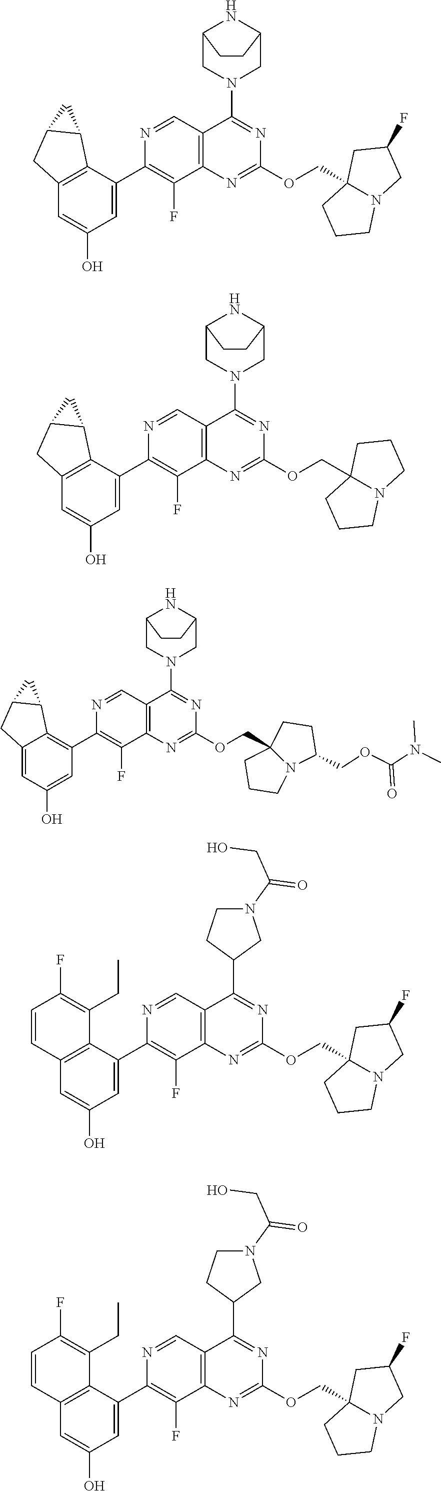 [SMILES: Oc1cc2c(c(-c3ncc4c(N5CC6CCC(C5)N6)nc(OC[C@@]56CCCN5C[C@H](F)C6)nc4c3F)c1)[C@H]1C[C@H]1C2 ; Oc1cc2c(c(-c3ncc4c(N5CC6CCC(C5)N6)nc(OCC56CCCN5CCC6)nc4c3F)c1)[C@H]1C[C@H]1C2 ; CN(C)C(=O)OC[C@H]1CC[C@@]2(COc3nc(N4CC5CCC(C4)N5)c4cnc(-c5cc(O)cc6c5[C@H]5C[C@H]5C6)c(F)c4n3)CCCN12 ; CCc1c(F)ccc2cc(O)cc(-c3ncc4c(C5CCN(C(=O)CO)C5)nc(OC[C@@]56CCCN5C[C@H](F)C6)nc4c3F)c12 ; CCc1c(F)ccc2cc(O)cc(-c3ncc4c(C5CCN(C(=O)CO)C5)nc(OC[C@@]56CCCN5C[C@H](F)C6)nc4c3F)c12]

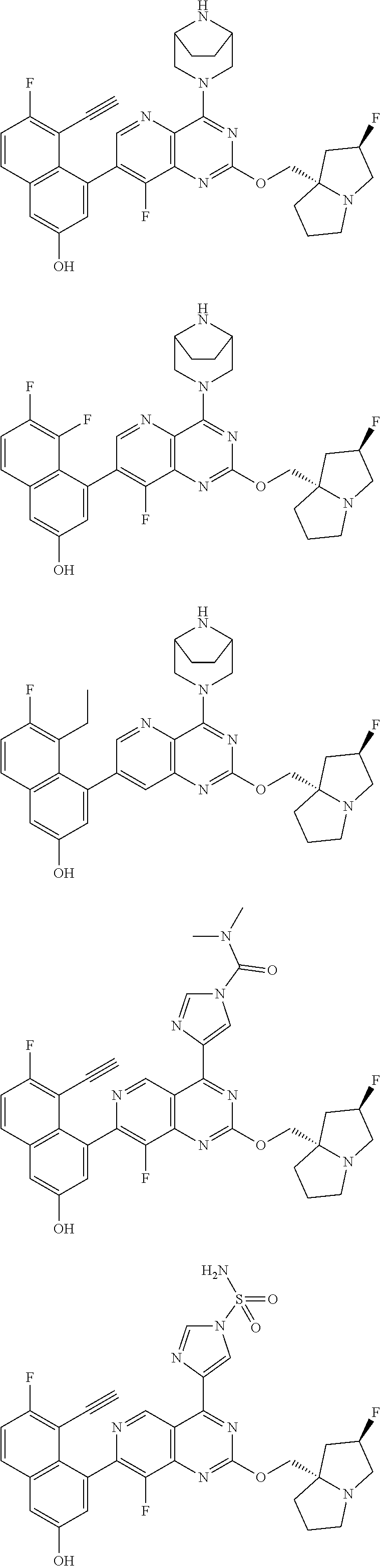 [SMILES: C#Cc1c(F)ccc2cc(O)cc(-c3cnc4c(N5CC6CCC(C5)N6)nc(OC[C@@]56CCCN5C[C@H](F)C6)nc4c3F)c12 ; Oc1cc(-c2cnc3c(N4CC5CCC(C4)N5)nc(OC[C@@]45CCCN4C[C@H](F)C5)nc3c2F)c2c(F)c(F)ccc2c1 ; CCc1c(F)ccc2cc(O)cc(-c3cnc4c(N5CC6CCC(C5)N6)nc(OC[C@@]56CCCN5C[C@H](F)C6)nc4c3)c12 ; C#Cc1c(F)ccc2cc(O)cc(-c3ncc4c(-c5cn(C(=O)N(C)C)cn5)nc(OC[C@@]56CCCN5C[C@H](F)C6)nc4c3F)c12 ; C#Cc1c(F)ccc2cc(O)cc(-c3ncc4c(-c5cn(S(N)(=O)=O)cn5)nc(OC[C@@]56CCCN5C[C@H](F)C6)nc4c3F)c12]

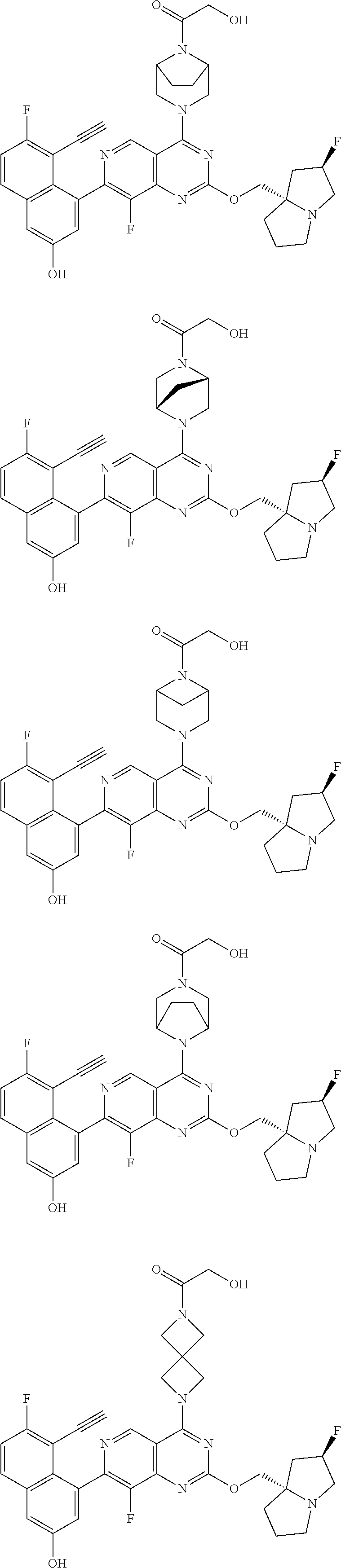 [SMILES: C#Cc1c(F)ccc2cc(O)cc(-c3ncc4c(N5CC6CCC(C5)N6C(=O)CO)nc(OC[C@@]56CCCN5C[C@H](F)C6)nc4c3F)c12 ; C#Cc1c(F)ccc2cc(O)cc(-c3ncc4c(N5C[C@@H]6C[C@H]5CN6C(=O)CO)nc(OC[C@@]56CCCN5C[C@H](F)C6)nc4c3F)c12 ; C#Cc1c(F)ccc2cc(O)cc(-c3ncc4c(N5CC6CC(C5)N6C(=O)CO)nc(OC[C@@]56CCCN5C[C@H](F)C6)nc4c3F)c12 ; C#Cc1c(F)ccc2cc(O)cc(-c3ncc4c(N5C6CCC5CN(C(=O)CO)C6)nc(OC[C@@]56CCCN5C[C@H](F)C6)nc4c3F)c12 ; C#Cc1c(F)ccc2cc(O)cc(-c3ncc4c(N5CC6(CN(C(=O)CO)C6)C5)nc(OC[C@@]56CCCN5C[C@H](F)C6)nc4c3F)c12]

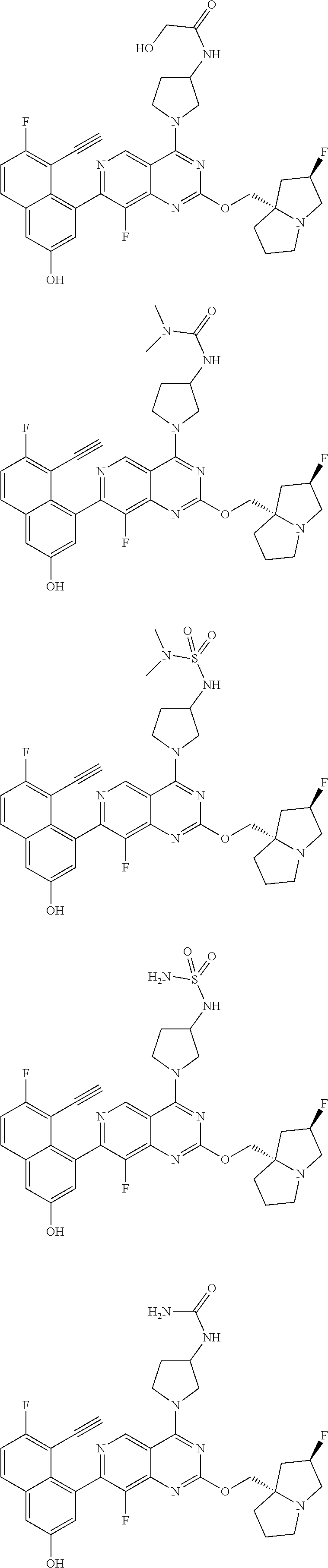 [SMILES: C#Cc1c(F)ccc2cc(O)cc(-c3ncc4c(N5CCC(NC(=O)CO)C5)nc(OC[C@@]56CCCN5C[C@H](F)C6)nc4c3F)c12 ; C#Cc1c(F)ccc2cc(O)cc(-c3ncc4c(N5CCC(NC(=O)N(C)C)C5)nc(OC[C@@]56CCCN5C[C@H](F)C6)nc4c3F)c12 ; C#Cc1c(F)ccc2cc(O)cc(-c3ncc4c(N5CCC(NS(=O)(=O)N(C)C)C5)nc(OC[C@@]56CCCN5C[C@H](F)C6)nc4c3F)c12 ; C#Cc1c(F)ccc2cc(O)cc(-c3ncc4c(N5CCC(NS(N)(=O)=O)C5)nc(OC[C@@]56CCCN5C[C@H](F)C6)nc4c3F)c12 ; C#Cc1c(F)ccc2cc(O)cc(-c3ncc4c(N5CCC(NC(N)=O)C5)nc(OC[C@@]56CCCN5C[C@H](F)C6)nc4c3F)c12]

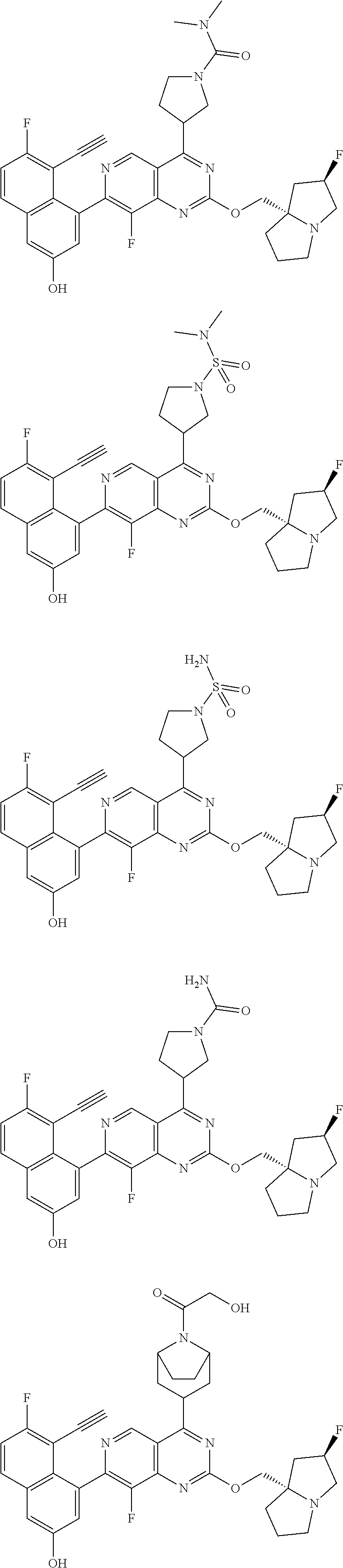 [SMILES: C#Cc1c(F)ccc2cc(O)cc(-c3ncc4c(C5CCN(C(=O)N(C)C)C5)nc(OC[C@@]56CCCN5C[C@H](F)C6)nc4c3F)c12 ; C#Cc1c(F)ccc2cc(O)cc(-c3ncc4c(C5CCN(S(=O)(=O)N(C)C)C5)nc(OC[C@@]56CCCN5C[C@H](F)C6)nc4c3F)c12 ; C#Cc1c(F)ccc2cc(O)cc(-c3ncc4c(C5CCN(S(N)(=O)=O)C5)nc(OC[C@@]56CCCN5C[C@H](F)C6)nc4c3F)c12 ; C#Cc1c(F)ccc2cc(O)cc(-c3ncc4c(C5CCN(C(N)=O)C5)nc(OC[C@@]56CCCN5C[C@H](F)C6)nc4c3F)c12 ; C#Cc1c(F)ccc2cc(O)cc(-c3ncc4c(C5CC6CCC(C5)N6C(=O)CO)nc(OC[C@@]56CCCN5C[C@H](F)C6)nc4c3F)c12]

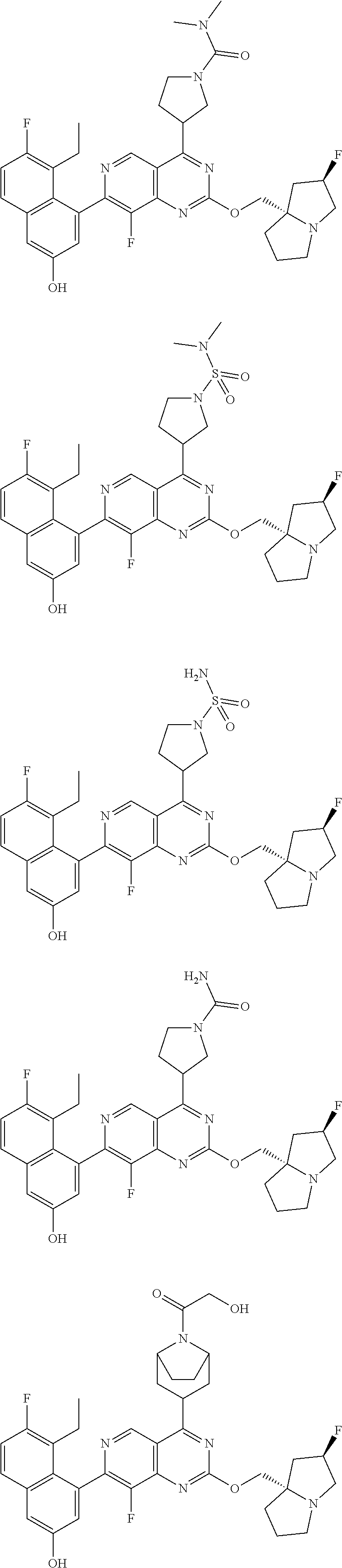 [SMILES: CCc1c(F)ccc2cc(O)cc(-c3ncc4c(C5CCN(C(=O)N(C)C)C5)nc(OC[C@@]56CCCN5C[C@H](F)C6)nc4c3F)c12 ; CCc1c(F)ccc2cc(O)cc(-c3ncc4c(C5CCN(S(=O)(=O)N(C)C)C5)nc(OC[C@@]56CCCN5C[C@H](F)C6)nc4c3F)c12 ; CCc1c(F)ccc2cc(O)cc(-c3ncc4c(C5CCN(S(N)(=O)=O)C5)nc(OC[C@@]56CCCN5C[C@H](F)C6)nc4c3F)c12 ; CCc1c(F)ccc2cc(O)cc(-c3ncc4c(C5CCN(C(N)=O)C5)nc(OC[C@@]56CCCN5C[C@H](F)C6)nc4c3F)c12 ; CCc1c(F)ccc2cc(O)cc(-c3ncc4c(C5CC6CCC(C5)N6C(=O)CO)nc(OC[C@@]56CCCN5C[C@H](F)C6)nc4c3F)c12]

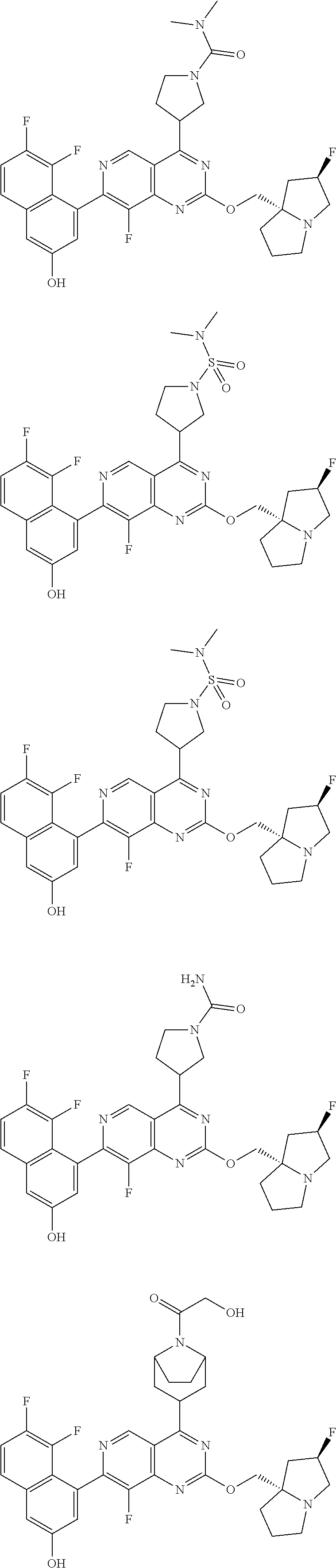 [SMILES: CN(C)C(=O)N1CCC(c2nc(OC[C@@]34CCCN3C[C@H](F)C4)nc3c(F)c(-c4cc(O)cc5ccc(F)c(F)c45)ncc23)C1 ; CN(C)S(=O)(=O)N1CCC(c2nc(OC[C@@]34CCCN3C[C@H](F)C4)nc3c(F)c(-c4cc(O)cc5ccc(F)c(F)c45)ncc23)C1 ; CN(C)S(=O)(=O)N1CCC(c2nc(OC[C@@]34CCCN3C[C@H](F)C4)nc3c(F)c(-c4cc(O)cc5ccc(F)c(F)c45)ncc23)C1 ; NC(=O)N1CCC(c2nc(OC[C@@]34CCCN3C[C@H](F)C4)nc3c(F)c(-c4cc(O)cc5ccc(F)c(F)c45)ncc23)C1 ; O=C(CO)N1C2CCC1CC(c1nc(OC[C@@]34CCCN3C[C@H](F)C4)nc3c(F)c(-c4cc(O)cc5ccc(F)c(F)c45)ncc13)C2]

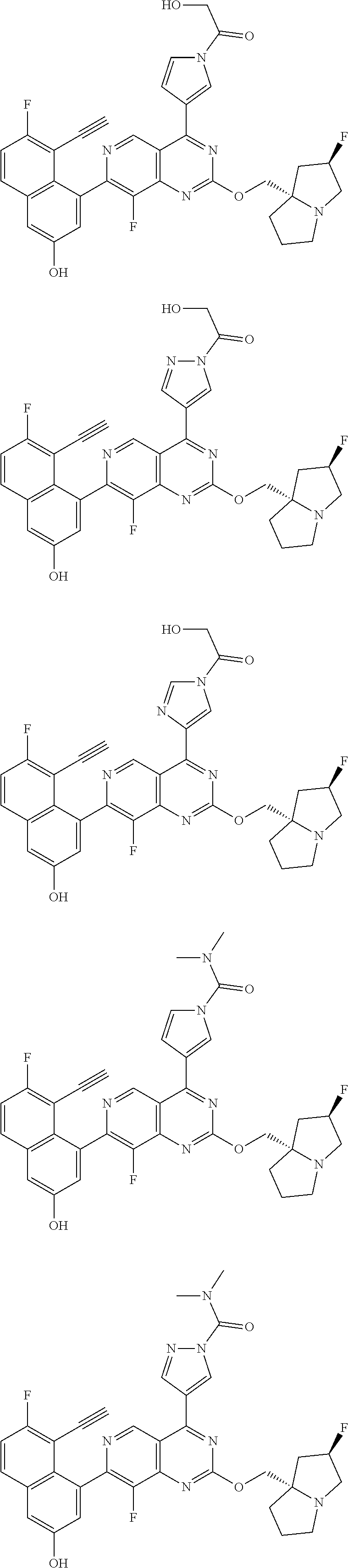 [SMILES: C#Cc1c(F)ccc2cc(O)cc(-c3ncc4c(-c5ccn(C(=O)CO)c5)nc(OC[C@@]56CCCN5C[C@H](F)C6)nc4c3F)c12 ; C#Cc1c(F)ccc2cc(O)cc(-c3ncc4c(-c5cnn(C(=O)CO)c5)nc(OC[C@@]56CCCN5C[C@H](F)C6)nc4c3F)c12 ; C#Cc1c(F)ccc2cc(O)cc(-c3ncc4c(-c5cn(C(=O)CO)cn5)nc(OC[C@@]56CCCN5C[C@H](F)C6)nc4c3F)c12 ; C#Cc1c(F)ccc2cc(O)cc(-c3ncc4c(-c5ccn(C(=O)N(C)C)c5)nc(OC[C@@]56CCCN5C[C@H](F)C6)nc4c3F)c12 ; C#Cc1c(F)ccc2cc(O)cc(-c3ncc4c(-c5cnn(C(=O)N(C)C)c5)nc(OC[C@@]56CCCN5C[C@H](F)C6)nc4c3F)c12]

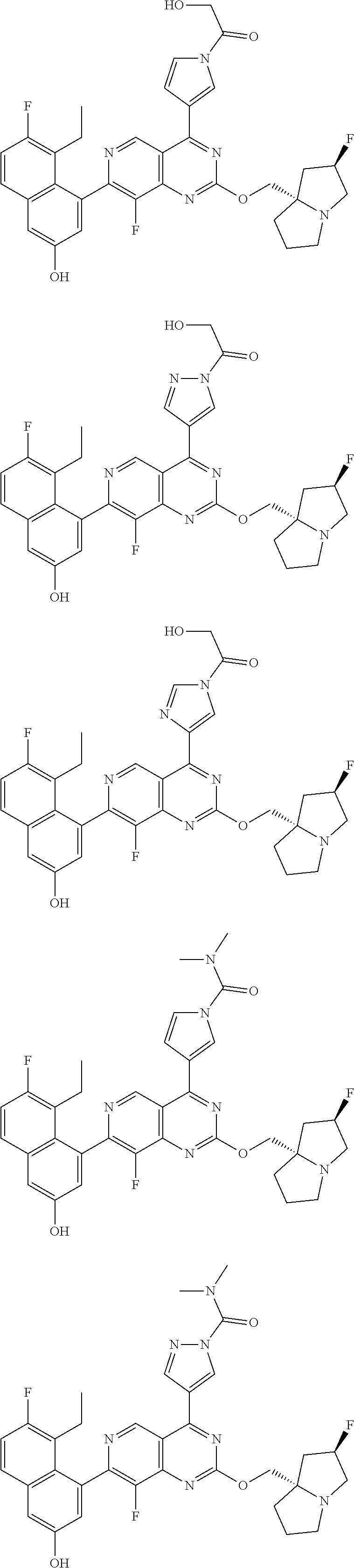 [SMILES: CCc1c(F)ccc2cc(O)cc(-c3ncc4c(-c5ccn(C(=O)CO)c5)nc(OC[C@@]56CCCN5C[C@H](F)C6)nc4c3F)c12 ; CCc1c(F)ccc2cc(O)cc(-c3ncc4c(-c5cnn(C(=O)CO)c5)nc(OC[C@@]56CCCN5C[C@H](F)C6)nc4c3F)c12 ; CCc1c(F)ccc2cc(O)cc(-c3ncc4c(-c5cn(C(=O)CO)cn5)nc(OC[C@@]56CCCN5C[C@H](F)C6)nc4c3F)c12 ; CCc1c(F)ccc2cc(O)cc(-c3ncc4c(-c5ccn(C(=O)N(C)C)c5)nc(OC[C@@]56CCCN5C[C@H](F)C6)nc4c3F)c12 ; CCc1c(F)ccc2cc(O)cc(-c3ncc4c(-c5cnn(C(=O)N(C)C)c5)nc(OC[C@@]56CCCN5C[C@H](F)C6)nc4c3F)c12]

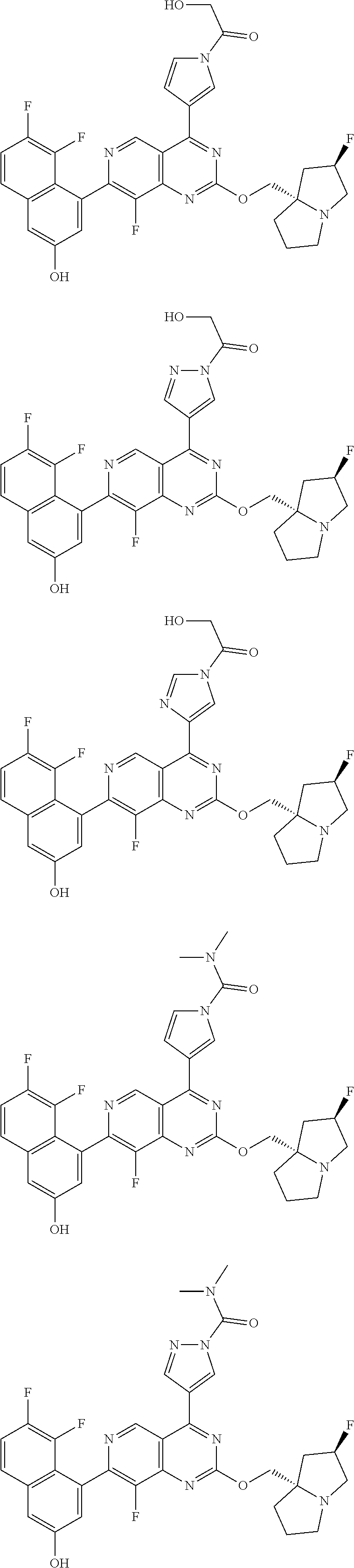 [SMILES: O=C(CO)n1ccc(-c2nc(OC[C@@]34CCCN3C[C@H](F)C4)nc3c(F)c(-c4cc(O)cc5ccc(F)c(F)c45)ncc23)c1 ; O=C(CO)n1cc(-c2nc(OC[C@@]34CCCN3C[C@H](F)C4)nc3c(F)c(-c4cc(O)cc5ccc(F)c(F)c45)ncc23)cn1 ; O=C(CO)n1cnc(-c2nc(OC[C@@]34CCCN3C[C@H](F)C4)nc3c(F)c(-c4cc(O)cc5ccc(F)c(F)c45)ncc23)c1 ; CN(C)C(=O)n1ccc(-c2nc(OC[C@@]34CCCN3C[C@H](F)C4)nc3c(F)c(-c4cc(O)cc5ccc(F)c(F)c45)ncc23)c1 ; CN(C)C(=O)n1cc(-c2nc(OC[C@@]34CCCN3C[C@H](F)C4)nc3c(F)c(-c4cc(O)cc5ccc(F)c(F)c45)ncc23)cn1]

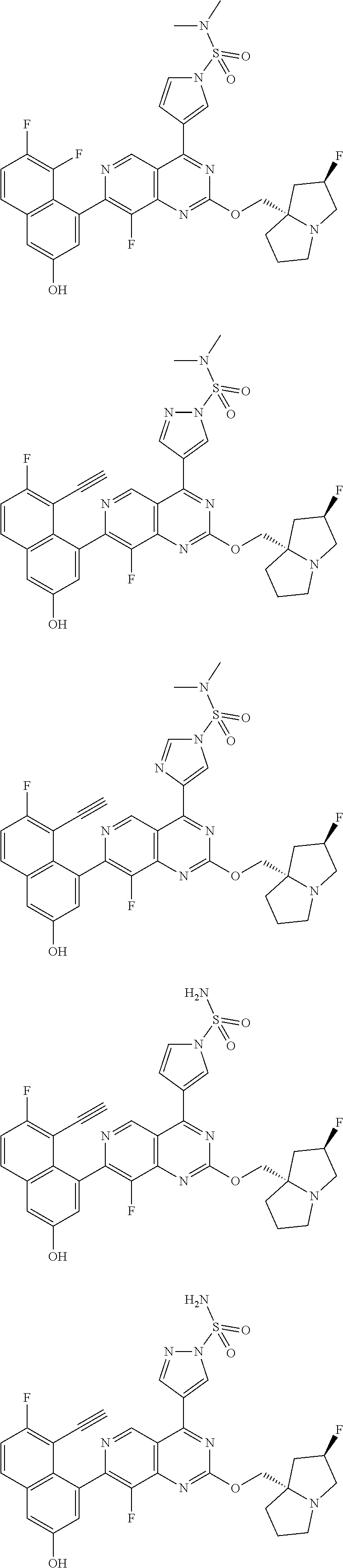 [SMILES: CN(C)S(=O)(=O)n1ccc(-c2nc(OC[C@@]34CCCN3C[C@H](F)C4)nc3c(F)c(-c4cc(O)cc5ccc(F)c(F)c45)ncc23)c1 ; C#Cc1c(F)ccc2cc(O)cc(-c3ncc4c(-c5cnn(S(=O)(=O)N(C)C)c5)nc(OC[C@@]56CCCN5C[C@H](F)C6)nc4c3F)c12 ; C#Cc1c(F)ccc2cc(O)cc(-c3ncc4c(-c5cn(S(=O)(=O)N(C)C)cn5)nc(OC[C@@]56CCCN5C[C@H](F)C6)nc4c3F)c12 ; C#Cc1c(F)ccc2cc(O)cc(-c3ncc4c(-c5ccn(S(N)(=O)=O)c5)nc(OC[C@@]56CCCN5C[C@H](F)C6)nc4c3F)c12 ; C#Cc1c(F)ccc2cc(O)cc(-c3ncc4c(-c5cnn(S(N)(=O)=O)c5)nc(OC[C@@]56CCCN5C[C@H](F)C6)nc4c3F)c12]

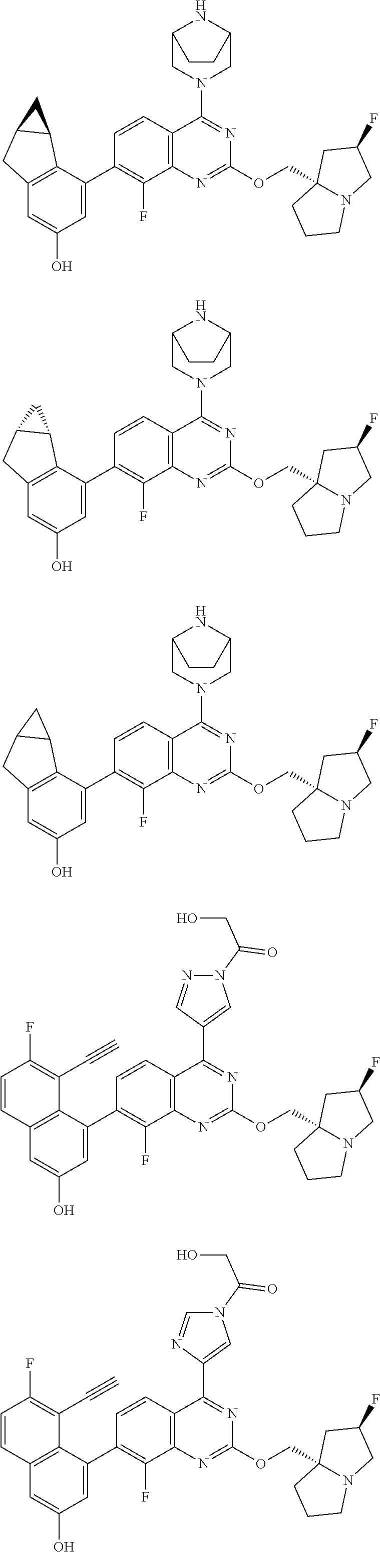 [SMILES: Oc1cc2c(c(-c3ccc4c(N5CC6CCC(C5)N6)nc(OC[C@@]56CCCN5C[C@H](F)C6)nc4c3F)c1)[C@@H]1C[C@@H]1C2 ; Oc1cc2c(c(-c3ccc4c(N5CC6CCC(C5)N6)nc(OC[C@@]56CCCN5C[C@H](F)C6)nc4c3F)c1)[C@H]1C[C@H]1C2 ; Oc1cc2c(c(-c3ccc4c(N5CC6CCC(C5)N6)nc(OC[C@@]56CCCN5C[C@H](F)C6)nc4c3F)c1)C1CC1C2 ; C#Cc1c(F)ccc2cc(O)cc(-c3ccc4c(-c5cnn(C(=O)CO)c5)nc(OC[C@@]56CCCN5C[C@H](F)C6)nc4c3F)c12 ; C#Cc1c(F)ccc2cc(O)cc(-c3ccc4c(-c5cn(C(=O)CO)cn5)nc(OC[C@@]56CCCN5C[C@H](F)C6)nc4c3F)c12]

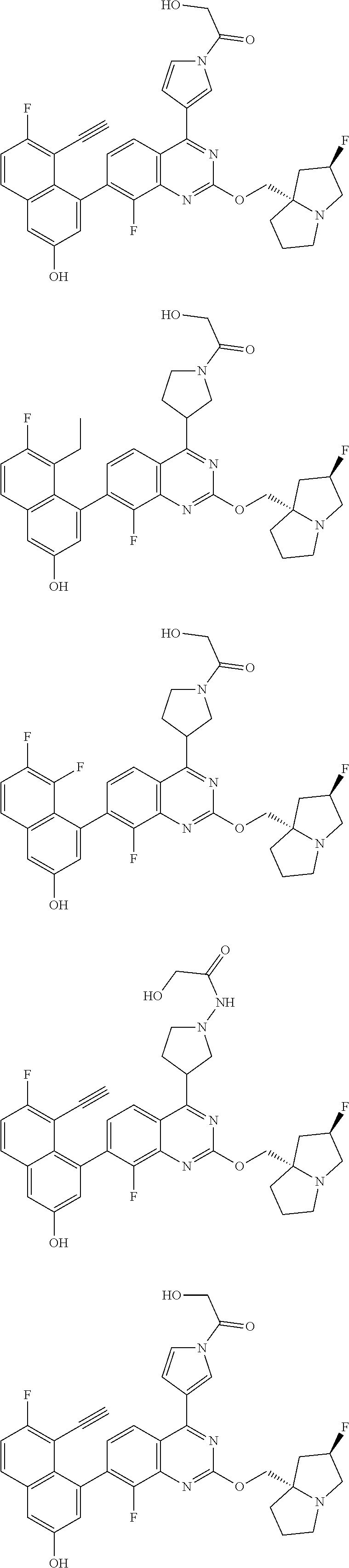 [SMILES: C#Cc1c(F)ccc2cc(O)cc(-c3ccc4c(-c5ccn(C(=O)CO)c5)nc(OC[C@@]56CCCN5C[C@H](F)C6)nc4c3F)c12 ; CCc1c(F)ccc2cc(O)cc(-c3ccc4c(C5CCN(C(=O)CO)C5)nc(OC[C@@]56CCCN5C[C@H](F)C6)nc4c3F)c12 ; O=C(CO)N1CCC(c2nc(OC[C@@]34CCCN3C[C@H](F)C4)nc3c(F)c(-c4cc(O)cc5ccc(F)c(F)c45)ccc23)C1 ; C#Cc1c(F)ccc2cc(O)cc(-c3ccc4c(C5CCN(NC(=O)CO)C5)nc(OC[C@@]56CCCN5C[C@H](F)C6)nc4c3F)c12 ; C#Cc1c(F)ccc2cc(O)cc(-c3ccc4c(-c5ccn(C(=O)CO)c5)nc(OC[C@@]56CCCN5C[C@H](F)C6)nc4c3F)c12]

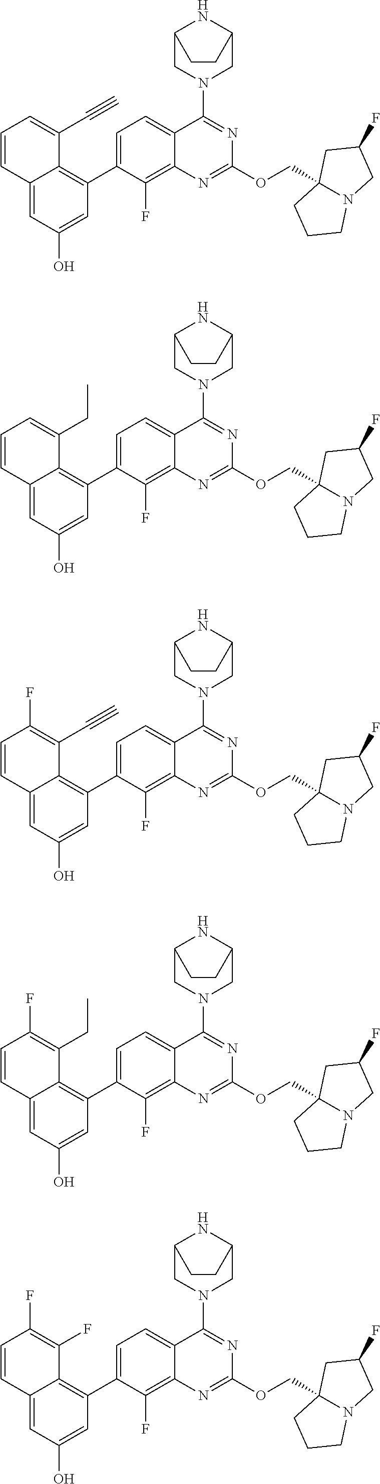 [SMILES: C#Cc1cccc2cc(O)cc(-c3ccc4c(N5CC6CCC(C5)N6)nc(OC[C@@]56CCCN5C[C@H](F)C6)nc4c3F)c12 ; CCc1cccc2cc(O)cc(-c3ccc4c(N5CC6CCC(C5)N6)nc(OC[C@@]56CCCN5C[C@H](F)C6)nc4c3F)c12 ; C#Cc1c(F)ccc2cc(O)cc(-c3ccc4c(N5CC6CCC(C5)N6)nc(OC[C@@]56CCCN5C[C@H](F)C6)nc4c3F)c12 ; CCc1c(F)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(F)c(F)ccc2c1]

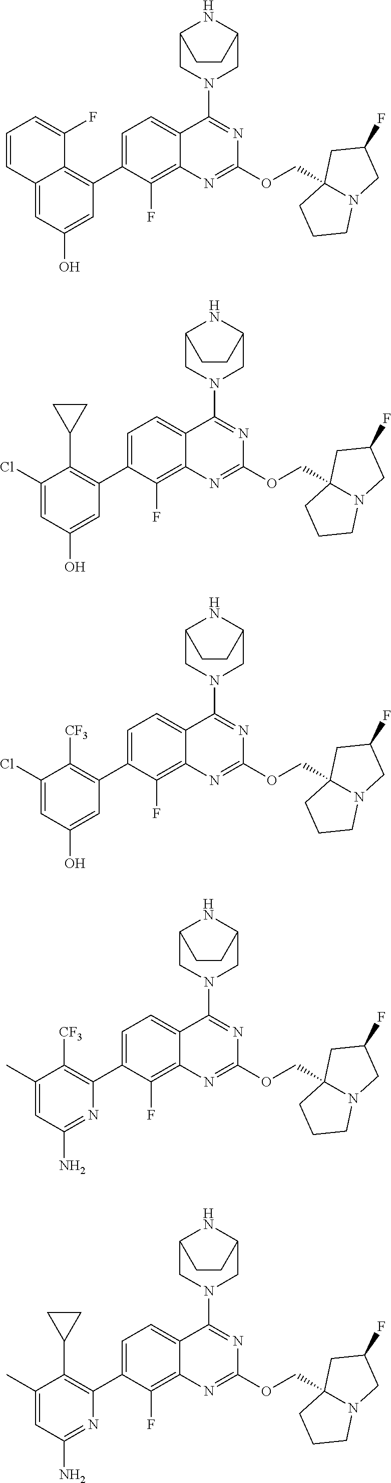 [SMILES: Oc1cc(-c2ccc3c(N4CC5CCC(C4)N5)nc(OC[C@@]45CCCN4C[C@H](F)C5)nc3c2F)c2c(F)cccc2c1 ; Oc1cc(Cl)c(C2CC2)c(-c2ccc3c(N4CC5CCC(C4)N5)nc(OC[C@@]45CCCN4C[C@H](F)C5)nc3c2F)c1 ; Oc1cc(Cl)c(C(F)(F)F)c(-c2ccc3c(N4CC5CCC(C4)N5)nc(OC[C@@]45CCCN4C[C@H](F)C5)nc3c2F)c1 ; Cc1cc(N)nc(-c2ccc3c(N4CC5CCC(C4)N5)nc(OC[C@@]45CCCN4C[C@H](F)C5)nc3c2F)c1C(F)(F)F ; Cc1cc(N)nc(-c2ccc3c(N4CC5CCC(C4)N5)nc(OC[C@@]45CCCN4C[C@H](F)C5)nc3c2F)c1C1CC1]

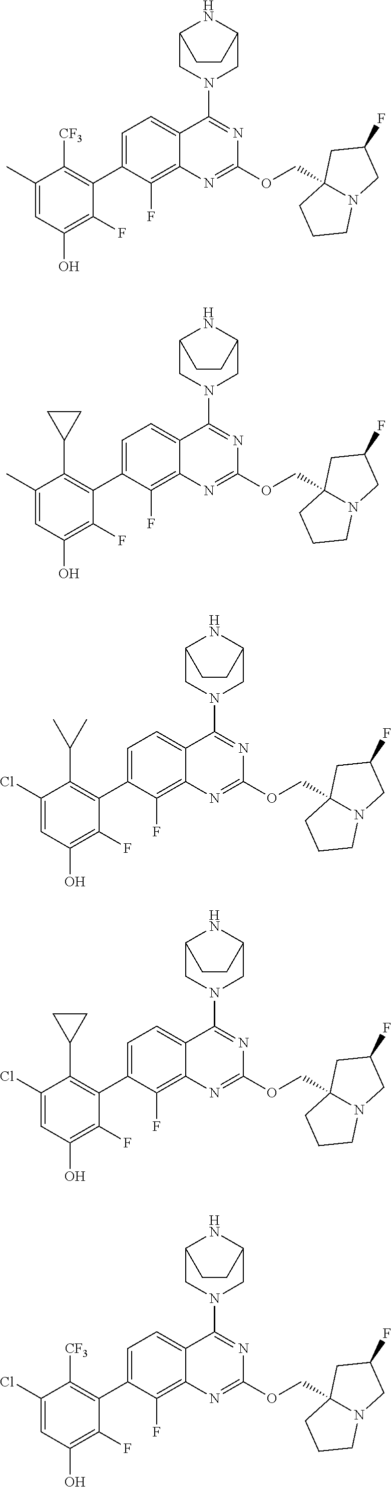 [SMILES: Cc1cc(O)c(F)c(-c2ccc3c(N4CC5CCC(C4)N5)nc(OC[C@@]45CCCN4C[C@H](F)C5)nc3c2F)c1C(F)(F)F ; Cc1cc(O)c(F)c(-c2ccc3c(N4CC5CCC(C4)N5)nc(OC[C@@]45CCCN4C[C@H](F)C5)nc3c2F)c1C1CC1 ; CC(C)c1c(Cl)cc(O)c(F)c1-c1ccc2c(N3CC4CCC(C3)N4)nc(OC[C@@]34CCCN3C[C@H](F)C4)nc2c1F ; Oc1cc(Cl)c(C2CC2)c(-c2ccc3c(N4CC5CCC(C4)N5)nc(OC[C@@]45CCCN4C[C@H](F)C5)nc3c2F)c1F ; Oc1cc(Cl)c(C(F)(F)F)c(-c2ccc3c(N4CC5CCC(C4)N5)nc(OC[C@@]45CCCN4C[C@H](F)C5)nc3c2F)c1F]

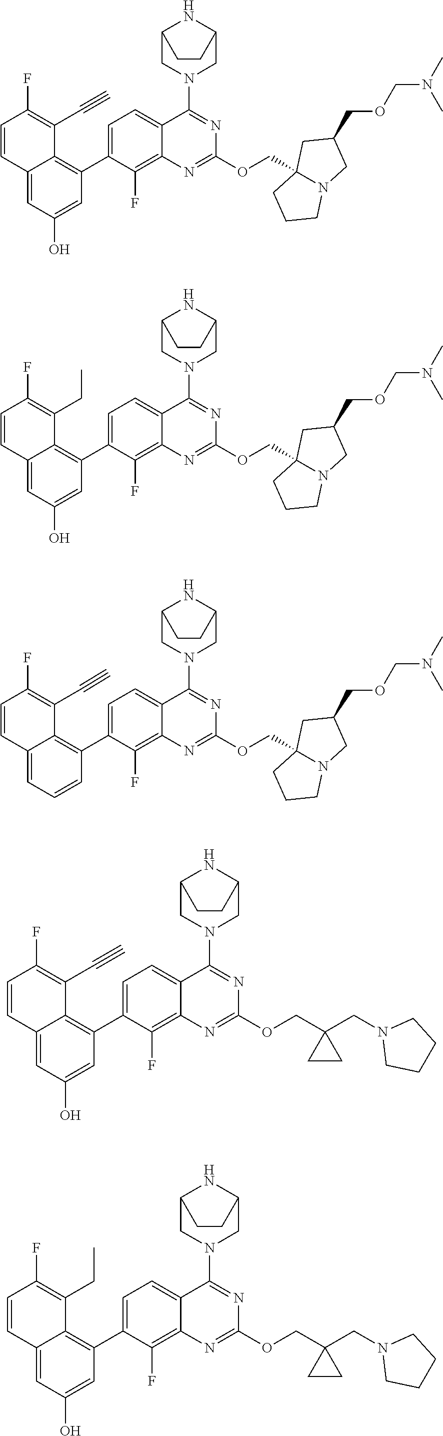 [SMILES: C#Cc1c(F)ccc2cc(O)cc(-c3ccc4c(N5CC6CCC(C5)N6)nc(OC[C@@]56CCCN5C[C@H](COCN(C)C)C6)nc4c3F)c12 ; CCc1c(F)ccc2cc(O)cc(-c3ccc4c(N5CC6CCC(C5)N6)nc(OC[C@@]56CCCN5C[C@H](COCN(C)C)C6)nc4c3F)c12 ; C#Cc1c(F)ccc2cccc(-c3ccc4c(N5CC6CCC(C5)N6)nc(OC[C@@]56CCCN5C[C@H](COCN(C)C)C6)nc4c3F)c12 ; C#Cc1c(F)ccc2cc(O)cc(-c3ccc4c(N5CC6CCC(C5)N6)nc(OCC5(CN6CCCC6)CC5)nc4c3F)c12 ; CCc1c(F)ccc2cc(O)cc(-c3ccc4c(N5CC6CCC(C5)N6)nc(OCC5(CN6CCCC6)CC5)nc4c3F)c12]

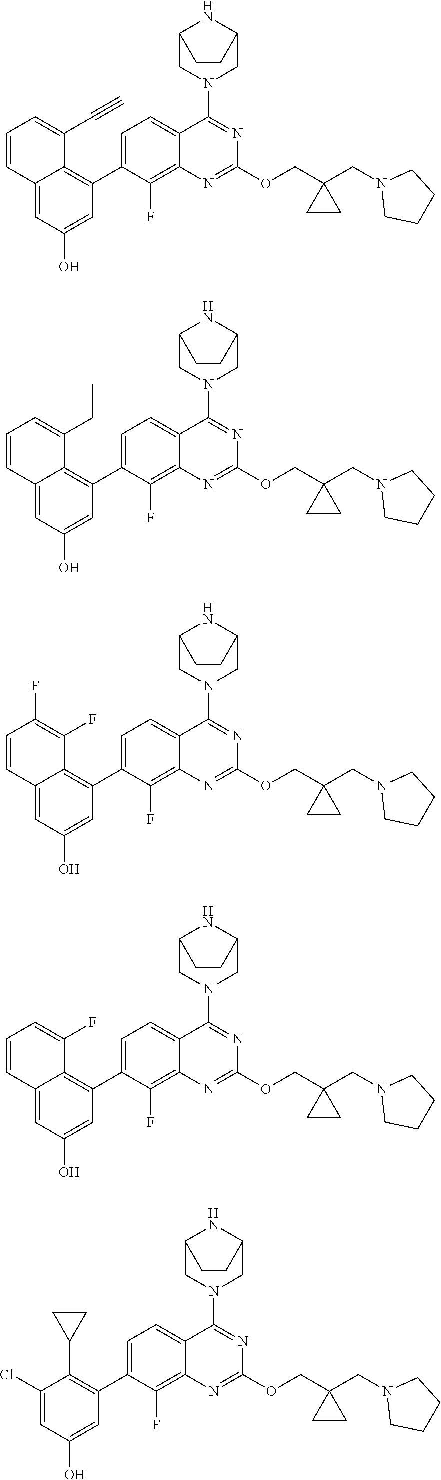 [SMILES: C#Cc1cccc2cc(O)cc(-c3ccc4c(N5CC6CCC(C5)N6)nc(OCC5(CN6CCCC6)CC5)nc4c3F)c12 ; CCc1cccc2cc(O)cc(-c3ccc4c(N5CC6CCC(C5)N6)nc(OCC5(CN6CCCC6)CC5)nc4c3F)c12 ; Oc1cc(-c2ccc3c(N4CC5CCC(C4)N5)nc(OCC4(CN5CCCC5)CC4)nc3c2F)c2c(F)c(F)ccc2c1 ; Oc1cc(-c2ccc3c(N4CC5CCC(C4)N5)nc(OCC4(CN5CCCC5)CC4)nc3c2F)c2c(F)cccc2c1 ; Oc1cc(Cl)c(C2CC2)c(-c2ccc3c(N4CC5CCC(C4)N5)nc(OCC4(CN5CCCC5)CC4)nc3c2F)c1]

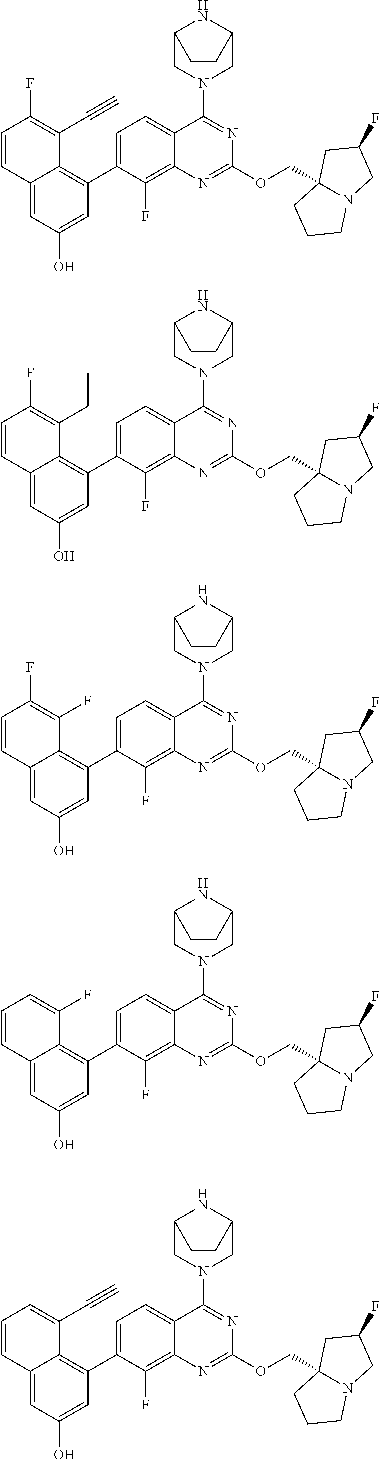 [SMILES: C#Cc1c(F)ccc2cc(O)cc(-c3ccc4c(N5CC6CCC(C5)N6)nc(OC[C@@]56CCCN5C[C@H](F)C6)nc4c3F)c12 ; CCc1c(F)ccc2cc(O)cc(-c3ccc4c(N5CC6CCC(C5)N6)nc(OC[C@@]56CCCN5C[C@H](F)C6)nc4c3F)c12 ; Oc1cc(-c2ccc3c(N4CC5CCC(C4)N5)nc(OC[C@@]45CCCN4C[C@H](F)C5)nc3c2F)c2c(F)c(F)ccc2c1 ; Oc1cc(-c2ccc3c(N4CC5CCC(C4)N5)nc(OC[C@@]45CCCN4C[C@H](F)C5)nc3c2F)c2c(F)cccc2c1 ; C#Cc1cccc2cc(O)cc(-c3ccc4c(N5CC6CCC(C5)N6)nc(OC[C@@]56CCCN5C[C@H](F)C6)nc4c3F)c12]

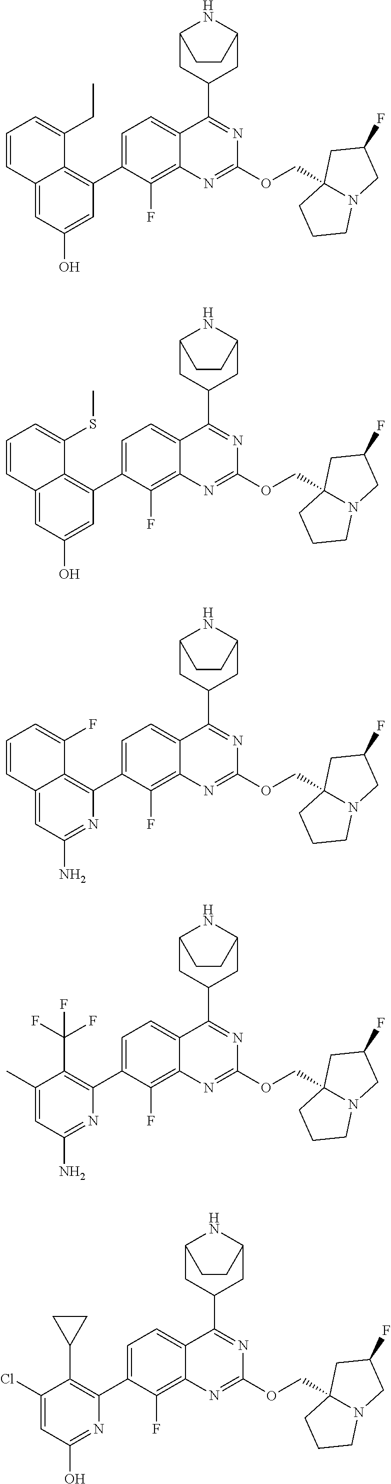 [SMILES: CCc1cccc2cc(O)cc(-c3ccc4c(C5CC6CCC(C5)N6)nc(OC[C@@]56CCCN5C[C@H](F)C6)nc4c3F)c12 ; CSc1cccc2cc(O)cc(-c3ccc4c(C5CC6CCC(C5)N6)nc(OC[C@@]56CCCN5C[C@H](F)C6)nc4c3F)c12 ; Nc1cc2cccc(F)c2c(-c2ccc3c(C4CC5CCC(C4)N5)nc(OC[C@@]45CCCN4C[C@H](F)C5)nc3c2F)n1 ; Cc1cc(N)nc(-c2ccc3c(C4CC5CCC(C4)N5)nc(OC[C@@]45CCCN4C[C@H](F)C5)nc3c2F)c1C(F)(F)F ; Oc1cc(Cl)c(C2CC2)c(-c2ccc3c(C4CC5CCC(C4)N5)nc(OC[C@@]45CCCN4C[C@H](F)C5)nc3c2F)n1]

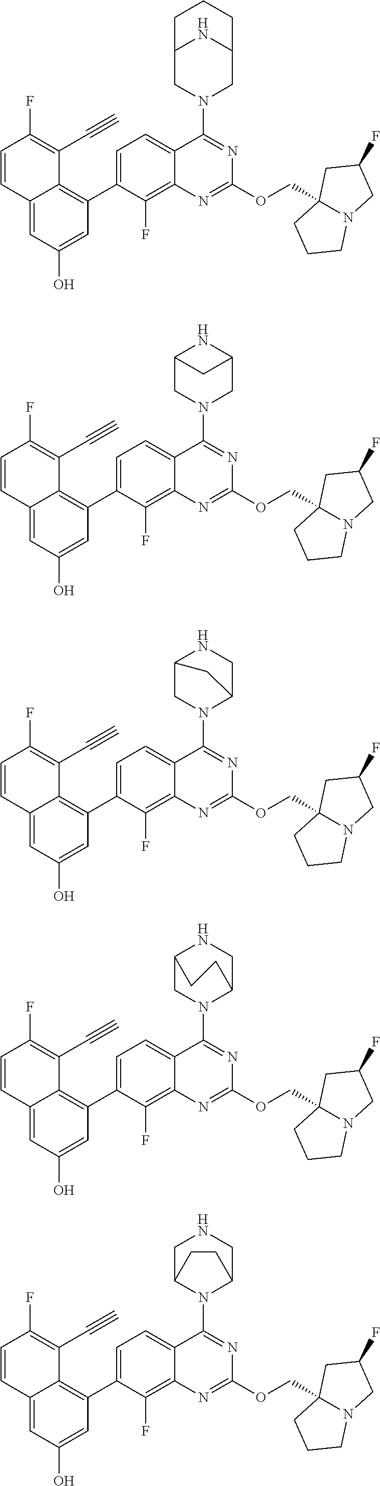 [SMILES: C#Cc1c(F)ccc2cc(O)cc(-c3ccc4c(N5CC6CCCC(C5)N6)nc(OC[C@@]56CCCN5C[C@H](F)C6)nc4c3F)c12 ; C#Cc1c(F)ccc2cc(O)cc(-c3ccc4c(N5CC6CC(C5)N6)nc(OC[C@@]56CCCN5C[C@H](F)C6)nc4c3F)c12 ; C#Cc1c(F)ccc2cc(O)cc(-c3ccc4c(N5CC6CC5CN6)nc(OC[C@@]56CCCN5C[C@H](F)C6)nc4c3F)c12 ; C#Cc1c(F)ccc2cc(O)cc(-c3ccc4c(N5CC6CCC5CN6)nc(OC[C@@]56CCCN5C[C@H](F)C6)nc4c3F)c12 ; C#Cc1c(F)ccc2cc(O)cc(-c3ccc4c(N5C6CCC5CNC6)nc(OC[C@@]56CCCN5C[C@H](F)C6)nc4c3F)c12]

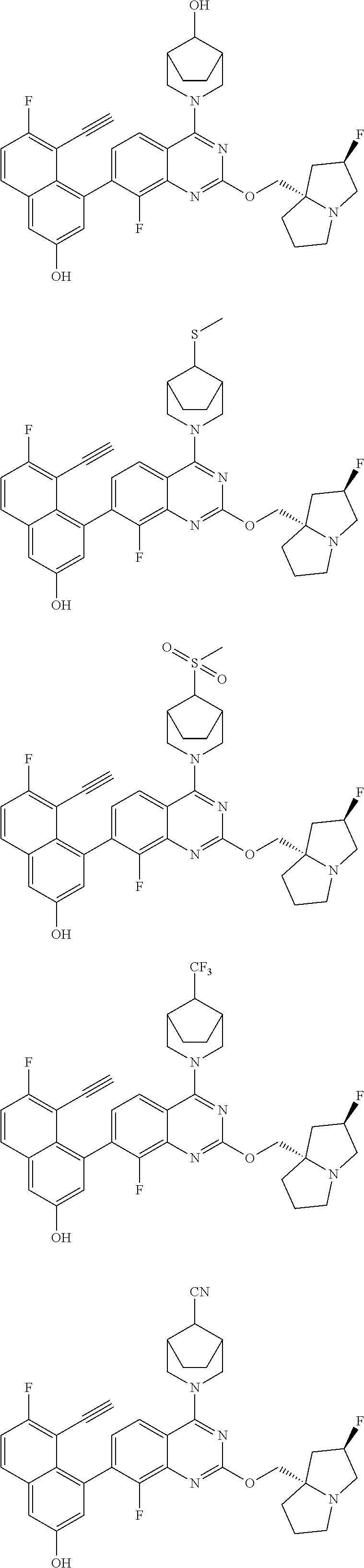 [SMILES: C#Cc1c(F)ccc2cc(O)cc(-c3ccc4c(N5CC6CCC(C5)C6O)nc(OC[C@@]56CCCN5C[C@H](F)C6)nc4c3F)c12 ; C#Cc1c(F)ccc2cc(O)cc(-c3ccc4c(N5CC6CCC(C5)C6SC)nc(OC[C@@]56CCCN5C[C@H](F)C6)nc4c3F)c12 ; C#Cc1c(F)ccc2cc(O)cc(-c3ccc4c(N5CC6CCC(C5)C6S(C)(=O)=O)nc(OC[C@@]56CCCN5C[C@H](F)C6)nc4c3F)c12 ; C#Cc1c(F)ccc2cc(O)cc(-c3ccc4c(N5CC6CCC(C5)C6C(F)(F)F)nc(OC[C@@]56CCCN5C[C@H](F)C6)nc4c3F)c12 ; C#Cc1c(F)ccc2cc(O)cc(-c3ccc4c(N5CC6CCC(C5)C6C#N)nc(OC[C@@]56CCCN5C[C@H](F)C6)nc4c3F)c12]

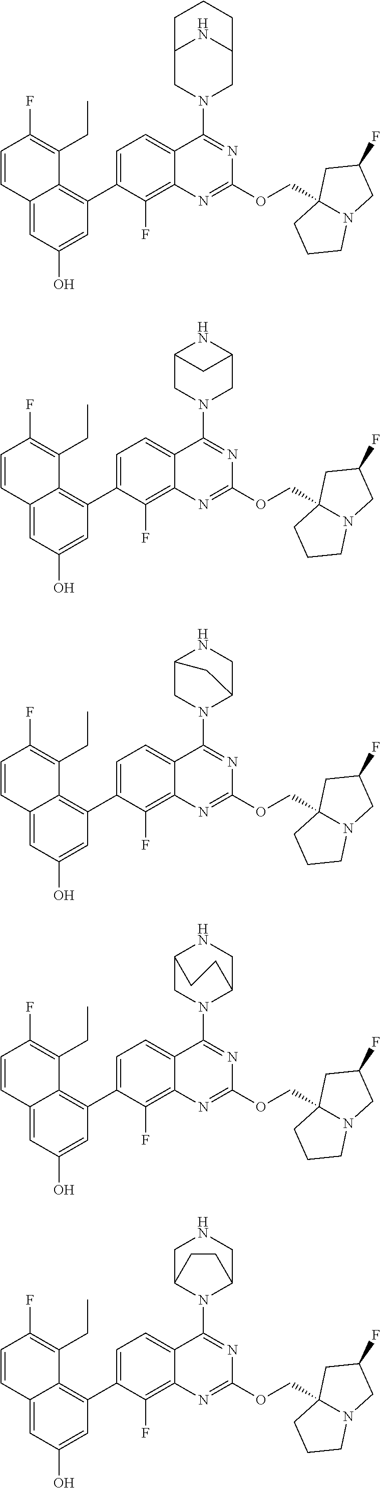 [SMILES: CCc1c(F)ccc2cc(O)cc(-c3ccc4c(N5CC6CCCC(C5)N6)nc(OC[C@@]56CCCN5C[C@H](F)C6)nc4c3F)c12 ; CCc1c(F)ccc2cc(O)cc(-c3ccc4c(N5CC6CC(C5)N6)nc(OC[C@@]56CCCN5C[C@H](F)C6)nc4c3F)c12 ; CCc1c(F)ccc2cc(O)cc(-c3ccc4c(N5CC6CC5CN6)nc(OC[C@@]56CCCN5C[C@H](F)C6)nc4c3F)c12 ; CCc1c(F)ccc2cc(O)cc(-c3ccc4c(N5CC6CCC5CN6)nc(OC[C@@]56CCCN5C[C@H](F)C6)nc4c3F)c12 ; CCc1c(F)ccc2cc(O)cc(-c3ccc4c(N5C6CCC5CNC6)nc(OC[C@@]56CCCN5C[C@H](F)C6)nc4c3F)c12]

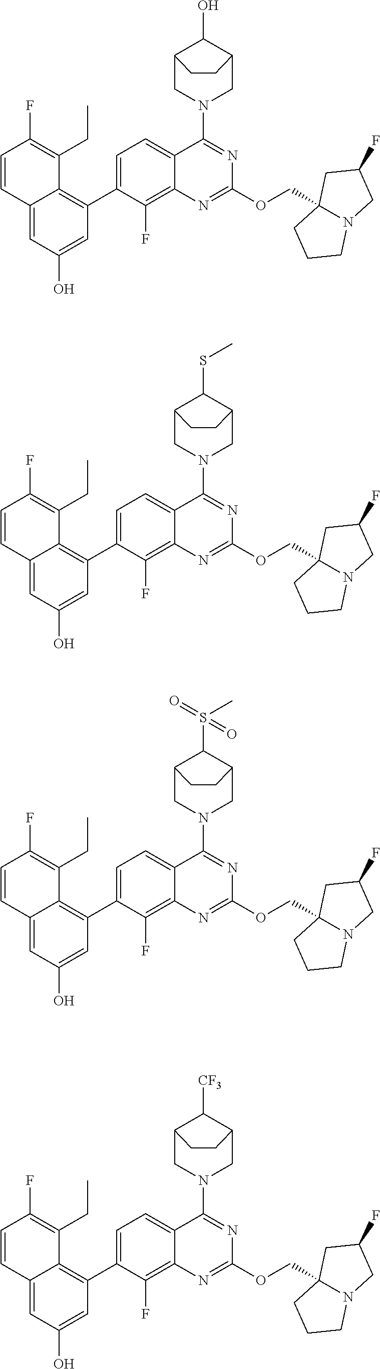 [SMILES: CCc1c(F)ccc2cc(O)cc(-c3ccc4c(N5CC6CCC(C5)C6O)nc(OC[C@@]56CCCN5C[C@H](F)C6)nc4c3F)c12 ; CCc1c(F)ccc2cc(O)cc(-c3ccc4c(N5CC6CCC(C5)C6SC)nc(OC[C@@]56CCCN5C[C@H](F)C6)nc4c3F)c12 ; CCc1c(F)ccc2cc(O)cc(-c3ccc4c(N5CC6CCC(C5)C6S(C)(=O)=O)nc(OC[C@@]56CCCN5C[C@H](F)C6)nc4c3F)c12 ; CCc1c(F)ccc2cc(O)cc(-c3ccc4c(N5CC6CCC(C5)C6C(F)(F)F)nc(OC[C@@]56CCCN5C[C@H](F)C6)nc4c3F)c12]

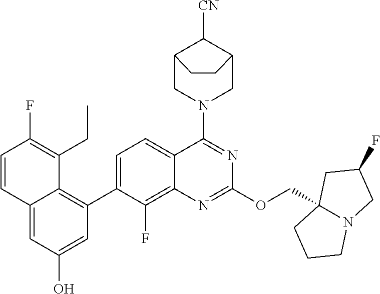 [SMILES: CCc1c(F)ccc2cc(O)cc(-c3ccc4c(N5CC6CCC(C5)C6C#N)nc(OC[C@@]56CCCN5C[C@H](F)C6)nc4c3F)c12]

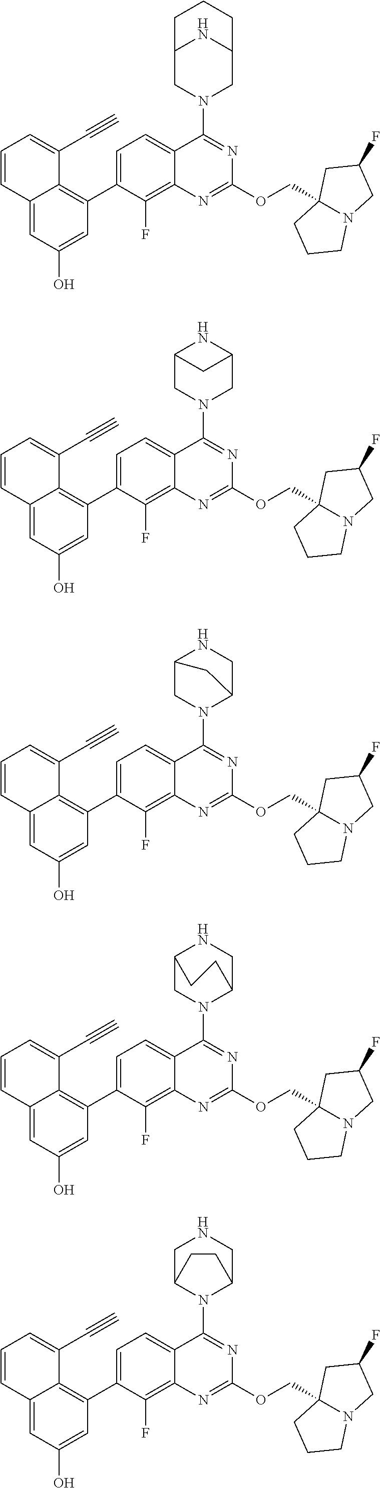 [SMILES: C#Cc1cccc2cc(O)cc(-c3ccc4c(N5CC6CCCC(C5)N6)nc(OC[C@@]56CCCN5C[C@H](F)C6)nc4c3F)c12 ; C#Cc1cccc2cc(O)cc(-c3ccc4c(N5CC6CC(C5)N6)nc(OC[C@@]56CCCN5C[C@H](F)C6)nc4c3F)c12 ; C#Cc1cccc2cc(O)cc(-c3ccc4c(N5CC6CC5CN6)nc(OC[C@@]56CCCN5C[C@H](F)C6)nc4c3F)c12 ; C#Cc1cccc2cc(O)cc(-c3ccc4c(N5CC6CCC5CN6)nc(OC[C@@]56CCCN5C[C@H](F)C6)nc4c3F)c12 ; C#Cc1cccc2cc(O)cc(-c3ccc4c(N5C6CCC5CNC6)nc(OC[C@@]56CCCN5C[C@H](F)C6)nc4c3F)c12]

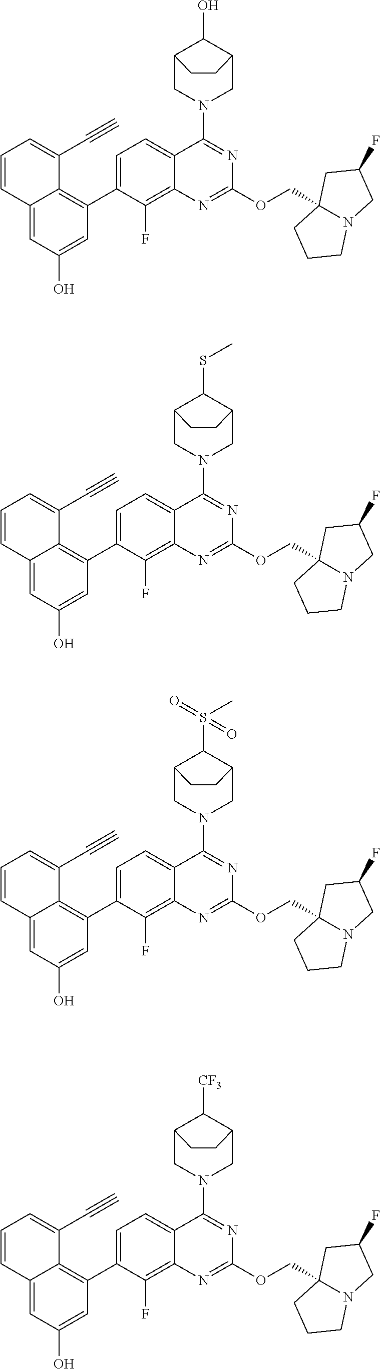 [SMILES: C#Cc1cccc2cc(O)cc(-c3ccc4c(N5CC6CCC(C5)C6O)nc(OC[C@@]56CCCN5C[C@H](F)C6)nc4c3F)c12 ; C#Cc1cccc2cc(O)cc(-c3ccc4c(N5CC6CCC(C5)C6SC)nc(OC[C@@]56CCCN5C[C@H](F)C6)nc4c3F)c12 ; C#Cc1cccc2cc(O)cc(-c3ccc4c(N5CC6CCC(C5)C6S(C)(=O)=O)nc(OC[C@@]56CCCN5C[C@H](F)C6)nc4c3F)c12 ; C#Cc1cccc2cc(O)cc(-c3ccc4c(N5CC6CCC(C5)C6C(F)(F)F)nc(OC[C@@]56CCCN5C[C@H](F)C6)nc4c3F)c12]

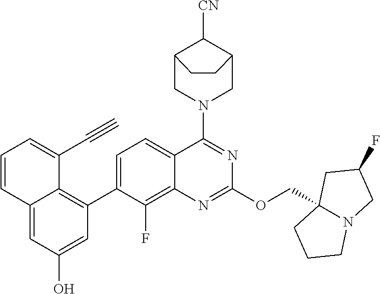 [SMILES: C#Cc1cccc2cc(O)cc(-c3ccc4c(N5CC6CCC(C5)C6C#N)nc(OC[C@@]56CCCN5C[C@H](F)C6)nc4c3F)c12]

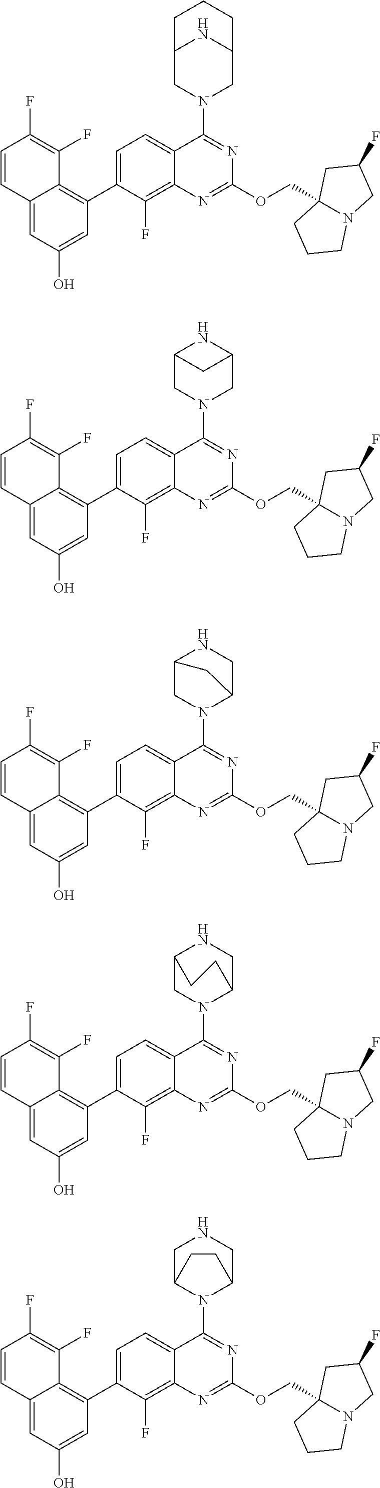 [SMILES: Oc1cc(-c2ccc3c(N4CC5CCCC(C4)N5)nc(OC[C@@]45CCCN4C[C@H](F)C5)nc3c2F)c2c(F)c(F)ccc2c1 ; Oc1cc(-c2ccc3c(N4CC5CC(C4)N5)nc(OC[C@@]45CCCN4C[C@H](F)C5)nc3c2F)c2c(F)c(F)ccc2c1 ; Oc1cc(-c2ccc3c(N4CC5CC4CN5)nc(OC[C@@]45CCCN4C[C@H](F)C5)nc3c2F)c2c(F)c(F)ccc2c1 ; Oc1cc(-c2ccc3c(N4CC5CCC4CN5)nc(OC[C@@]45CCCN4C[C@H](F)C5)nc3c2F)c2c(F)c(F)ccc2c1 ; Oc1cc(-c2ccc3c(N4C5CCC4CNC5)nc(OC[C@@]45CCCN4C[C@H](F)C5)nc3c2F)c2c(F)c(F)ccc2c1]

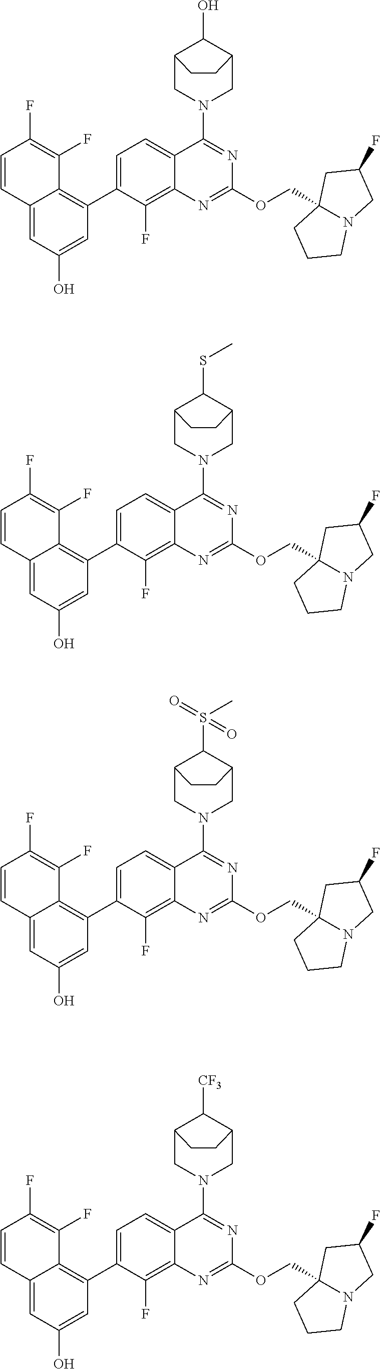 [SMILES: Oc1cc(-c2ccc3c(N4CC5CCC(C4)C5O)nc(OC[C@@]45CCCN4C[C@H](F)C5)nc3c2F)c2c(F)c(F)ccc2c1 ; CSC1C2CCC1CN(c1nc(OC[C@@]34CCCN3C[C@H](F)C4)nc3c(F)c(-c4cc(O)cc5ccc(F)c(F)c45)ccc13)C2 ; CS(=O)(=O)C1C2CCC1CN(c1nc(OC[C@@]34CCCN3C[C@H](F)C4)nc3c(F)c(-c4cc(O)cc5ccc(F)c(F)c45)ccc13)C2 ; Oc1cc(-c2ccc3c(N4CC5CCC(C4)C5C(F)(F)F)nc(OC[C@@]45CCCN4C[C@H](F)C5)nc3c2F)c2c(F)c(F)ccc2c1]

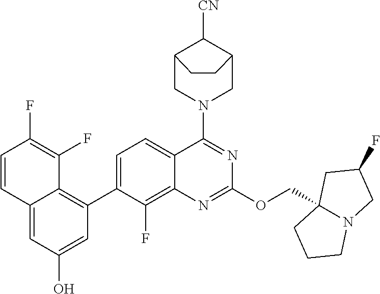 [SMILES: N#CC1C2CCC1CN(c1nc(OC[C@@]34CCCN3C[C@H](F)C4)nc3c(F)c(-c4cc(O)cc5ccc(F)c(F)c45)ccc13)C2]

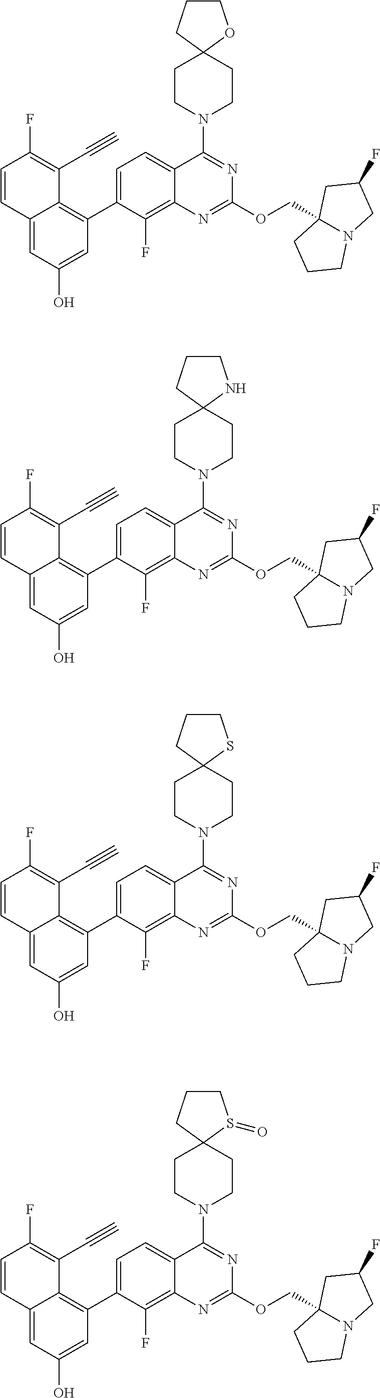 [SMILES: C#Cc1c(F)ccc2cc(O)cc(-c3ccc4c(N5CCC6(CCCO6)CC5)nc(OC[C@@]56CCCN5C[C@H](F)C6)nc4c3F)c12 ; C#Cc1c(F)ccc2cc(O)cc(-c3ccc4c(N5CCC6(CCCN6)CC5)nc(OC[C@@]56CCCN5C[C@H](F)C6)nc4c3F)c12 ; C#Cc1c(F)ccc2cc(O)cc(-c3ccc4c(N5CCC6(CCCS6)CC5)nc(OC[C@@]56CCCN5C[C@H](F)C6)nc4c3F)c12 ; C#Cc1c(F)ccc2cc(O)cc(-c3ccc4c(N5CCC6(CCCS6=O)CC5)nc(OC[C@@]56CCCN5C[C@H](F)C6)nc4c3F)c12]

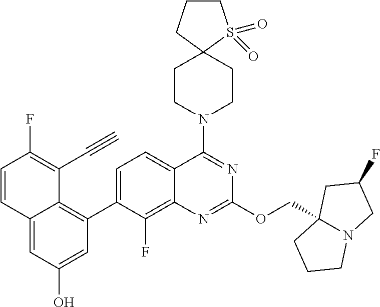 [SMILES: C#Cc1c(F)ccc2cc(O)cc(-c3ccc4c(N5CCC6(CCCS6(=O)=O)CC5)nc(OC[C@@]56CCCN5C[C@H](F)C6)nc4c3F)c12]

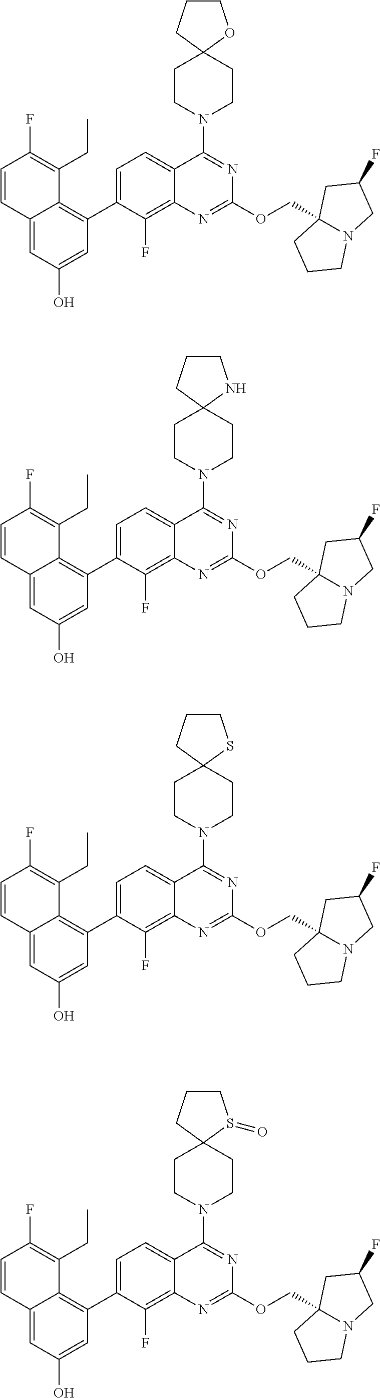 [SMILES: CCc1c(F)ccc2cc(O)cc(-c3ccc4c(N5CCC6(CCCO6)CC5)nc(OC[C@@]56CCCN5C[C@H](F)C6)nc4c3F)c12 ; CCc1c(F)ccc2cc(O)cc(-c3ccc4c(N5CCC6(CCCN6)CC5)nc(OC[C@@]56CCCN5C[C@H](F)C6)nc4c3F)c12 ; CCc1c(F)ccc2cc(O)cc(-c3ccc4c(N5CCC6(CCCS6)CC5)nc(OC[C@@]56CCCN5C[C@H](F)C6)nc4c3F)c12 ; CCc1c(F)ccc2cc(O)cc(-c3ccc4c(N5CCC6(CCCS6=O)CC5)nc(OC[C@@]56CCCN5C[C@H](F)C6)nc4c3F)c12]

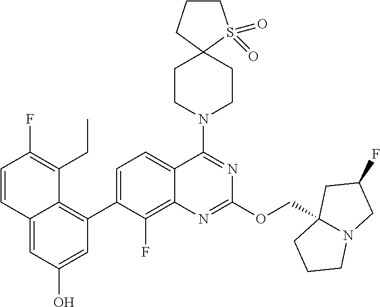 [SMILES: CCc1c(F)ccc2cc(O)cc(-c3ccc4c(N5CCC6(CCCS6(=O)=O)CC5)nc(OC[C@@]56CCCN5C[C@H](F)C6)nc4c3F)c12]

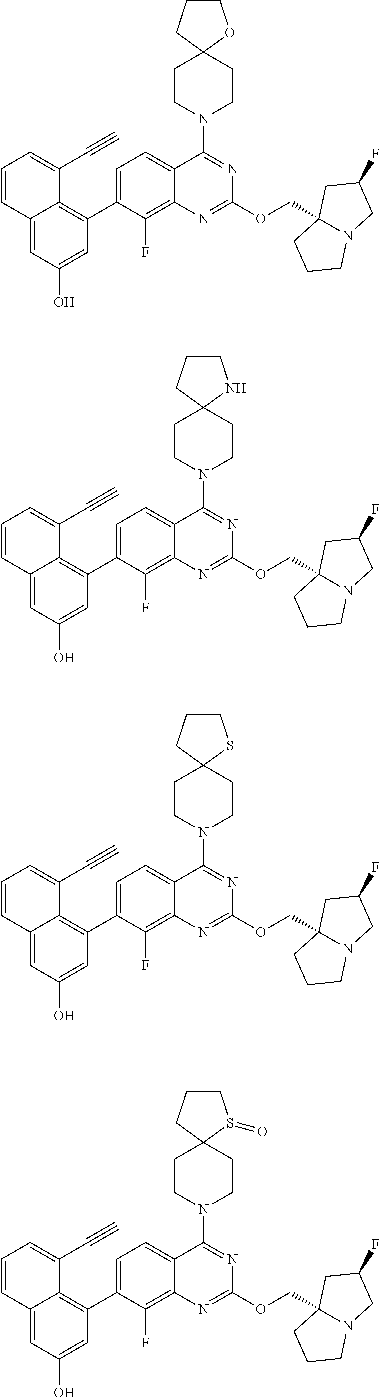 [SMILES: C#Cc1cccc2cc(O)cc(-c3ccc4c(N5CCC6(CCCO6)CC5)nc(OC[C@@]56CCCN5C[C@H](F)C6)nc4c3F)c12 ; C#Cc1cccc2cc(O)cc(-c3ccc4c(N5CCC6(CCCN6)CC5)nc(OC[C@@]56CCCN5C[C@H](F)C6)nc4c3F)c12 ; C#Cc1cccc2cc(O)cc(-c3ccc4c(N5CCC6(CCCS6)CC5)nc(OC[C@@]56CCCN5C[C@H](F)C6)nc4c3F)c12 ; C#Cc1cccc2cc(O)cc(-c3ccc4c(N5CCC6(CCCS6=O)CC5)nc(OC[C@@]56CCCN5C[C@H](F)C6)nc4c3F)c12]

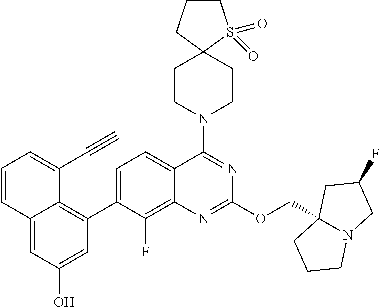 [SMILES: C#Cc1cccc2cc(O)cc(-c3ccc4c(N5CCC6(CCCS6(=O)=O)CC5)nc(OC[C@@]56CCCN5C[C@H](F)C6)nc4c3F)c12]

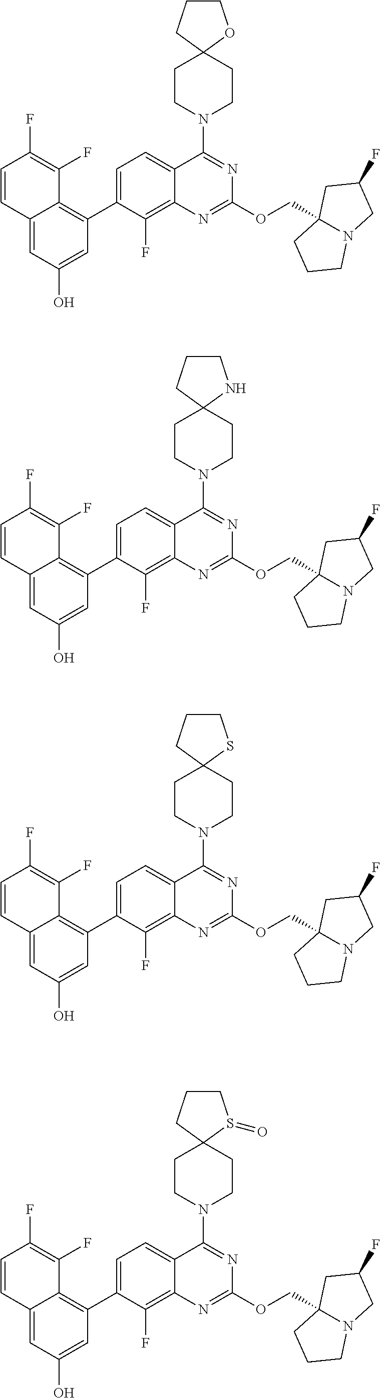 [SMILES: Oc1cc(-c2ccc3c(N4CCC5(CCCO5)CC4)nc(OC[C@@]45CCCN4C[C@H](F)C5)nc3c2F)c2c(F)c(F)ccc2c1 ; Oc1cc(-c2ccc3c(N4CCC5(CCCN5)CC4)nc(OC[C@@]45CCCN4C[C@H](F)C5)nc3c2F)c2c(F)c(F)ccc2c1 ; Oc1cc(-c2ccc3c(N4CCC5(CCCS5)CC4)nc(OC[C@@]45CCCN4C[C@H](F)C5)nc3c2F)c2c(F)c(F)ccc2c1 ; O=S1CCCC12CCN(c1nc(OC[C@@]34CCCN3C[C@H](F)C4)nc3c(F)c(-c4cc(O)cc5ccc(F)c(F)c45)ccc13)CC2]

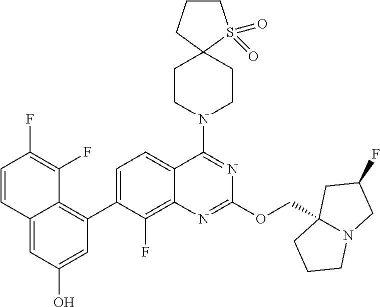 [SMILES: O=S1(=O)CCCC12CCN(c1nc(OC[C@@]34CCCN3C[C@H](F)C4)nc3c(F)c(-c4cc(O)cc5ccc(F)c(F)c45)ccc13)CC2]

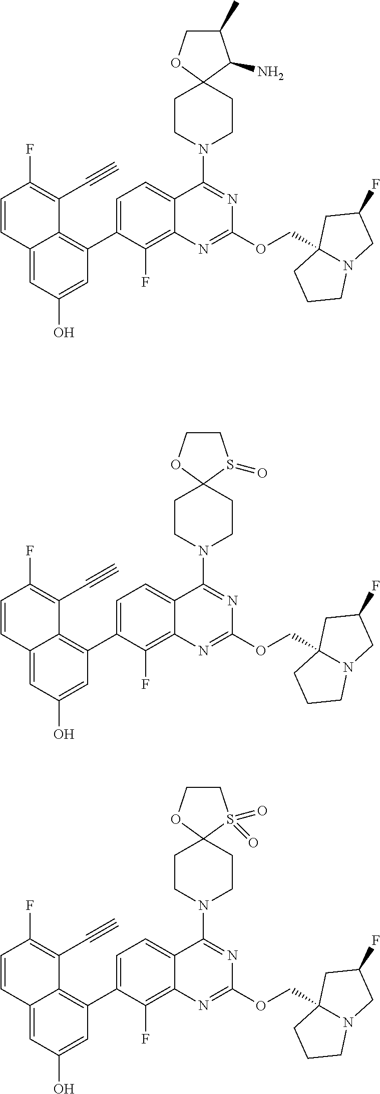 [SMILES: C#Cc1c(F)ccc2cc(O)cc(-c3ccc4c(N5CCC6(CC5)OC[C@@H](C)[C@H]6N)nc(OC[C@@]56CCCN5C[C@H](F)C6)nc4c3F)c12 ; C#Cc1c(F)ccc2cc(O)cc(-c3ccc4c(N5CCC6(CC5)OCCS6=O)nc(OC[C@@]56CCCN5C[C@H](F)C6)nc4c3F)c12 ; C#Cc1c(F)ccc2cc(O)cc(-c3ccc4c(N5CCC6(CC5)OCCS6(=O)=O)nc(OC[C@@]56CCCN5C[C@H](F)C6)nc4c3F)c12]

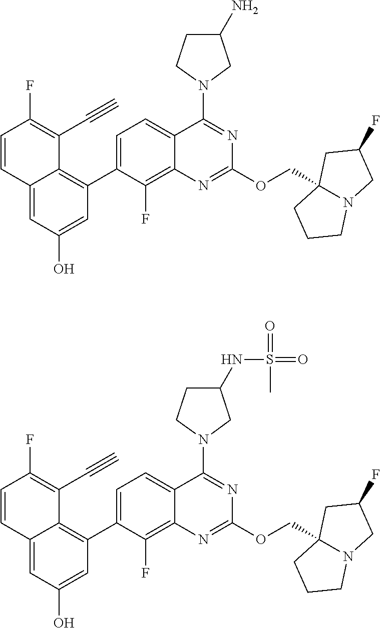 [SMILES: C#Cc1c(F)ccc2cc(O)cc(-c3ccc4c(N5CCC(N)C5)nc(OC[C@@]56CCCN5C[C@H](F)C6)nc4c3F)c12 ; C#Cc1c(F)ccc2cc(O)cc(-c3ccc4c(N5CCC(NS(C)(=O)=O)C5)nc(OC[C@@]56CCCN5C[C@H](F)C6)nc4c3F)c12]

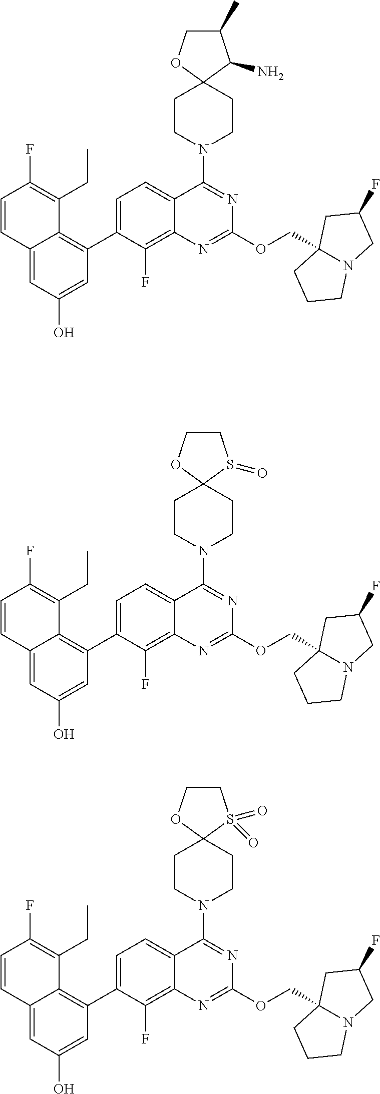 [SMILES: CCc1c(F)ccc2cc(O)cc(-c3ccc4c(N5CCC6(CC5)OC[C@@H](C)[C@H]6N)nc(OC[C@@]56CCCN5C[C@H](F)C6)nc4c3F)c12 ; CCc1c(F)ccc2cc(O)cc(-c3ccc4c(N5CCC6(CC5)OCCS6=O)nc(OC[C@@]56CCCN5C[C@H](F)C6)nc4c3F)c12 ; CCc1c(F)ccc2cc(O)cc(-c3ccc4c(N5CCC6(CC5)OCCS6(=O)=O)nc(OC[C@@]56CCCN5C[C@H](F)C6)nc4c3F)c12]

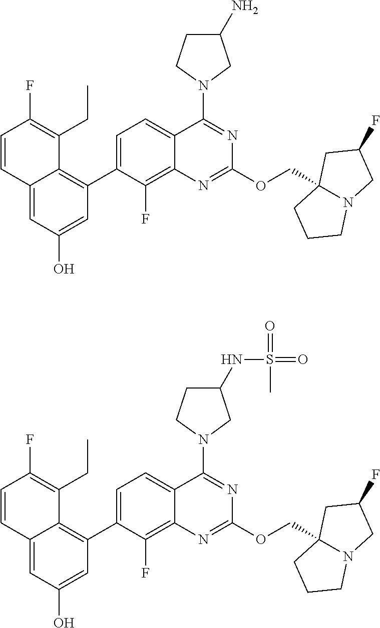 [SMILES: CCc1c(F)ccc2cc(O)cc(-c3ccc4c(N5CCC(N)C5)nc(OC[C@@]56CCCN5C[C@H](F)C6)nc4c3F)c12 ; CCc1c(F)ccc2cc(O)cc(-c3ccc4c(N5CCC(NS(C)(=O)=O)C5)nc(OC[C@@]56CCCN5C[C@H](F)C6)nc4c3F)c12]

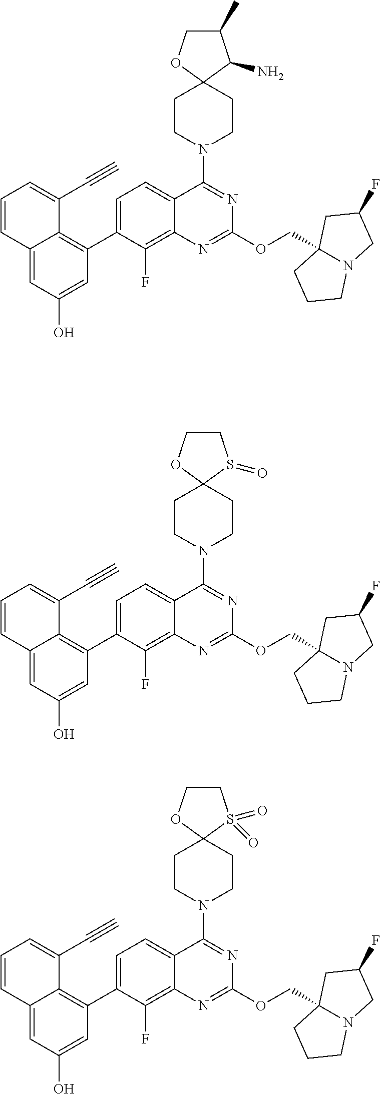 [SMILES: C#Cc1cccc2cc(O)cc(-c3ccc4c(N5CCC6(CC5)OC[C@@H](C)[C@H]6N)nc(OC[C@@]56CCCN5C[C@H](F)C6)nc4c3F)c12 ; C#Cc1cccc2cc(O)cc(-c3ccc4c(N5CCC6(CC5)OCCS6=O)nc(OC[C@@]56CCCN5C[C@H](F)C6)nc4c3F)c12 ; C#Cc1cccc2cc(O)cc(-c3ccc4c(N5CCC6(CC5)OCCS6(=O)=O)nc(OC[C@@]56CCCN5C[C@H](F)C6)nc4c3F)c12]

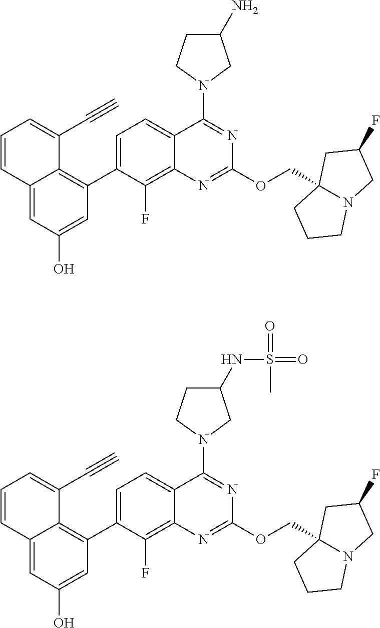 [SMILES: C#Cc1cccc2cc(O)cc(-c3ccc4c(N5CCC(N)C5)nc(OC[C@@]56CCCN5C[C@H](F)C6)nc4c3F)c12 ; C#Cc1cccc2cc(O)cc(-c3ccc4c(N5CCC(NS(C)(=O)=O)C5)nc(OC[C@@]56CCCN5C[C@H](F)C6)nc4c3F)c12]

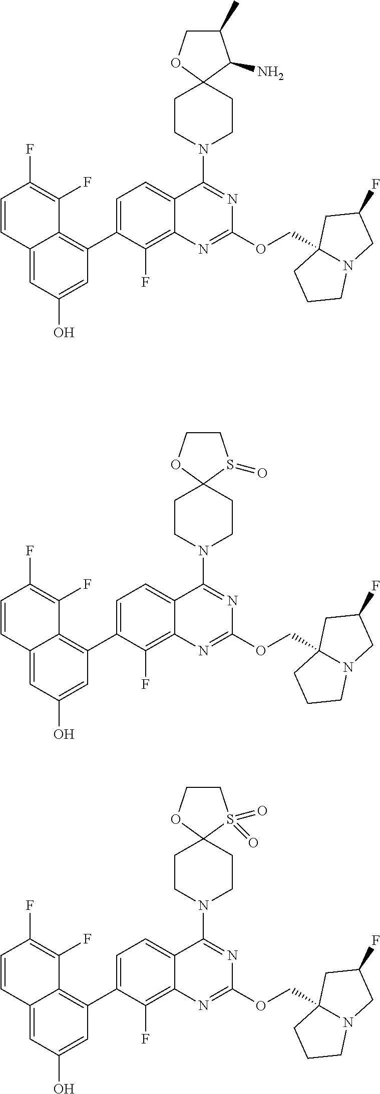 [SMILES: C[C@@H]1COC2(CCN(c3nc(OC[C@@]45CCCN4C[C@H](F)C5)nc4c(F)c(-c5cc(O)cc6ccc(F)c(F)c56)ccc34)CC2)[C@@H]1N ; O=S1CCOC12CCN(c1nc(OC[C@@]34CCCN3C[C@H](F)C4)nc3c(F)c(-c4cc(O)cc5ccc(F)c(F)c45)ccc13)CC2 ; O=S1(=O)CCOC12CCN(c1nc(OC[C@@]34CCCN3C[C@H](F)C4)nc3c(F)c(-c4cc(O)cc5ccc(F)c(F)c45)ccc13)CC2]

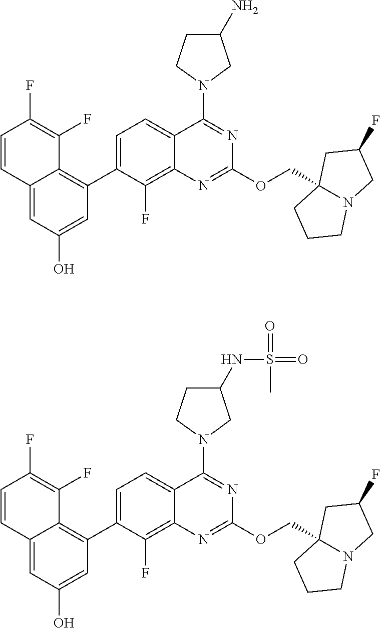 [SMILES: NC1CCN(c2nc(OC[C@@]34CCCN3C[C@H](F)C4)nc3c(F)c(-c4cc(O)cc5ccc(F)c(F)c45)ccc23)C1 ; CS(=O)(=O)NC1CCN(c2nc(OC[C@@]34CCCN3C[C@H](F)C4)nc3c(F)c(-c4cc(O)cc5ccc(F)c(F)c45)ccc23)C1]

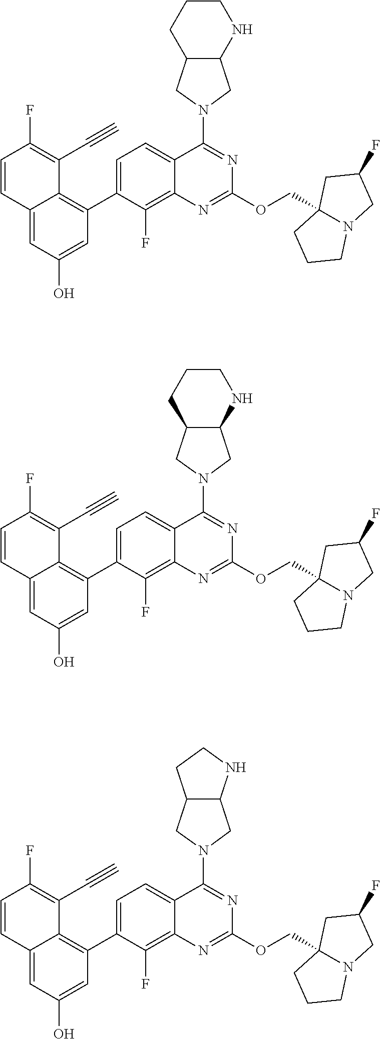 [SMILES: C#Cc1c(F)ccc2cc(O)cc(-c3ccc4c(N5CC6CCCNC6C5)nc(OC[C@@]56CCCN5C[C@H](F)C6)nc4c3F)c12 ; C#Cc1c(F)ccc2cc(O)cc(-c3ccc4c(N5C[C@H]6CCCN[C@H]6C5)nc(OC[C@@]56CCCN5C[C@H](F)C6)nc4c3F)c12 ; C#Cc1c(F)ccc2cc(O)cc(-c3ccc4c(N5CC6CCNC6C5)nc(OC[C@@]56CCCN5C[C@H](F)C6)nc4c3F)c12]

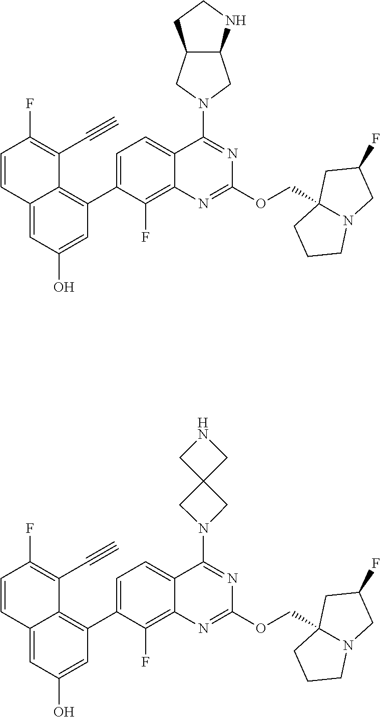 [SMILES: C#Cc1c(F)ccc2cc(O)cc(-c3ccc4c(N5C[C@H]6CCN[C@H]6C5)nc(OC[C@@]56CCCN5C[C@H](F)C6)nc4c3F)c12 ; C#Cc1c(F)ccc2cc(O)cc(-c3ccc4c(N5CC6(CNC6)C5)nc(OC[C@@]56CCCN5C[C@H](F)C6)nc4c3F)c12]

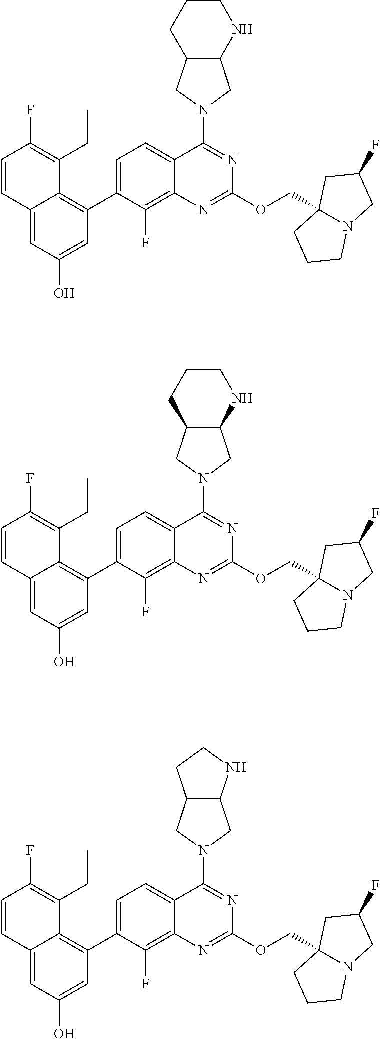 [SMILES: CCc1c(F)ccc2cc(O)cc(-c3ccc4c(N5CC6CCCNC6C5)nc(OC[C@@]56CCCN5C[C@H](F)C6)nc4c3F)c12 ; CCc1c(F)ccc2cc(O)cc(-c3ccc4c(N5C[C@H]6CCCN[C@H]6C5)nc(OC[C@@]56CCCN5C[C@H](F)C6)nc4c3F)c12 ; CCc1c(F)ccc2cc(O)cc(-c3ccc4c(N5CC6CCNC6C5)nc(OC[C@@]56CCCN5C[C@H](F)C6)nc4c3F)c12]

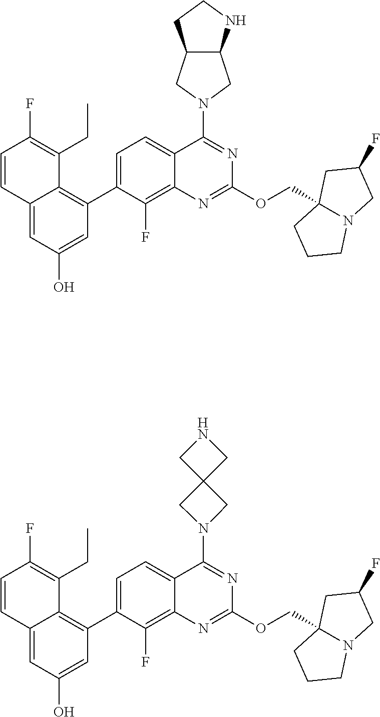 [SMILES: CCc1c(F)ccc2cc(O)cc(-c3ccc4c(N5C[C@H]6CCN[C@H]6C5)nc(OC[C@@]56CCCN5C[C@H](F)C6)nc4c3F)c12 ; CCc1c(F)ccc2cc(O)cc(-c3ccc4c(N5CC6(CNC6)C5)nc(OC[C@@]56CCCN5C[C@H](F)C6)nc4c3F)c12]

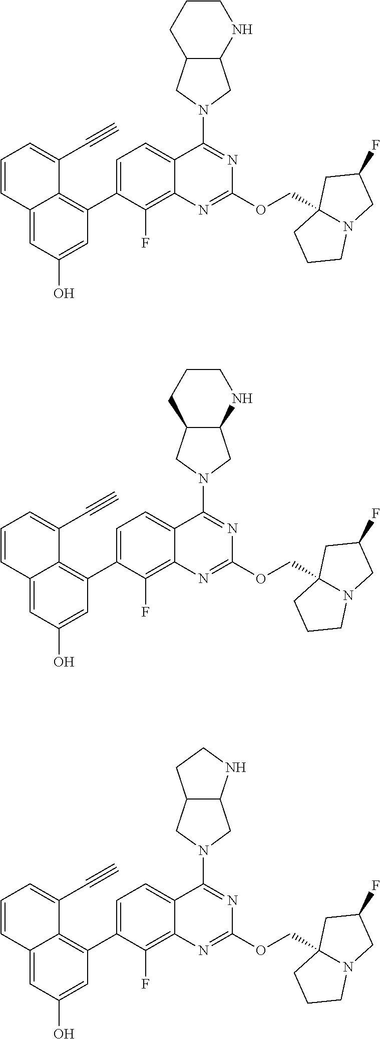 [SMILES: C#Cc1cccc2cc(O)cc(-c3ccc4c(N5CC6CCCNC6C5)nc(OC[C@@]56CCCN5C[C@H](F)C6)nc4c3F)c12 ; C#Cc1cccc2cc(O)cc(-c3ccc4c(N5C[C@H]6CCCN[C@H]6C5)nc(OC[C@@]56CCCN5C[C@H](F)C6)nc4c3F)c12 ; C#Cc1cccc2cc(O)cc(-c3ccc4c(N5CC6CCNC6C5)nc(OC[C@@]56CCCN5C[C@H](F)C6)nc4c3F)c12]

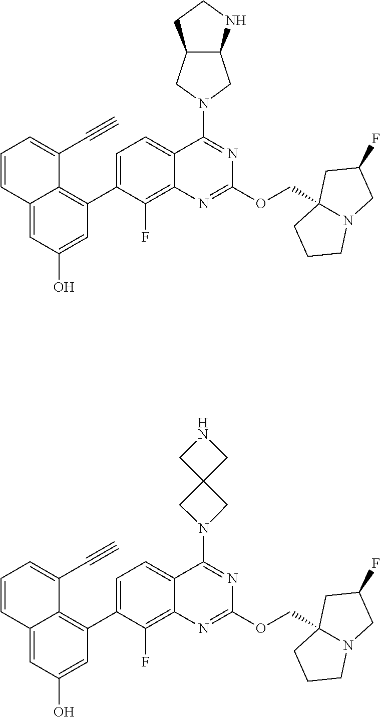 [SMILES: C#Cc1cccc2cc(O)cc(-c3ccc4c(N5C[C@H]6CCN[C@H]6C5)nc(OC[C@@]56CCCN5C[C@H](F)C6)nc4c3F)c12 ; C#Cc1cccc2cc(O)cc(-c3ccc4c(N5CC6(CNC6)C5)nc(OC[C@@]56CCCN5C[C@H](F)C6)nc4c3F)c12]

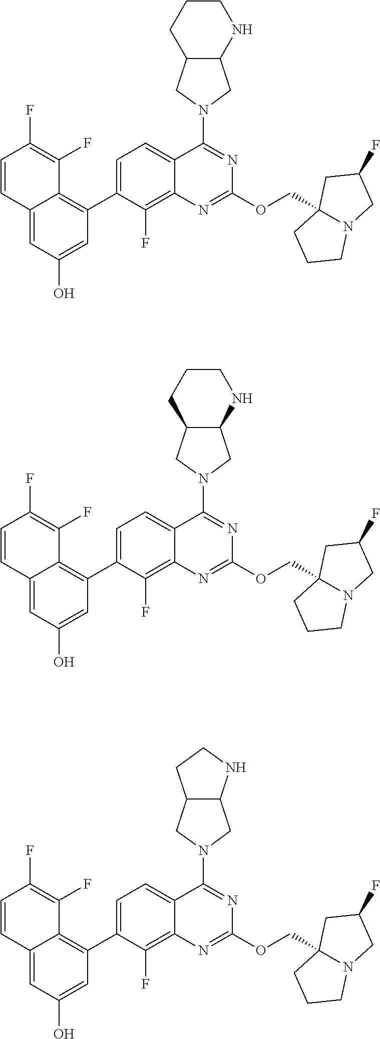 [SMILES: Oc1cc(-c2ccc3c(N4CC5CCCNC5C4)nc(OC[C@@]45CCCN4C[C@H](F)C5)nc3c2F)c2c(F)c(F)ccc2c1 ; Oc1cc(-c2ccc3c(N4C[C@H]5CCCN[C@H]5C4)nc(OC[C@@]45CCCN4C[C@H](F)C5)nc3c2F)c2c(F)c(F)ccc2c1 ; Oc1cc(-c2ccc3c(N4CC5CCNC5C4)nc(OC[C@@]45CCCN4C[C@H](F)C5)nc3c2F)c2c(F)c(F)ccc2c1]

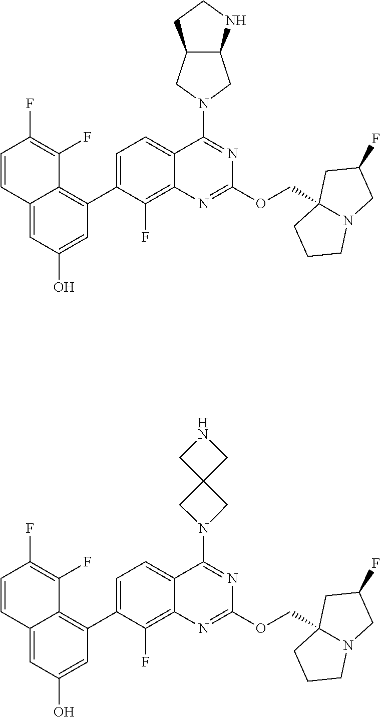 [SMILES: Oc1cc(-c2ccc3c(N4C[C@H]5CCN[C@H]5C4)nc(OC[C@@]45CCCN4C[C@H](F)C5)nc3c2F)c2c(F)c(F)ccc2c1 ; Oc1cc(-c2ccc3c(N4CC5(CNC5)C4)nc(OC[C@@]45CCCN4C[C@H](F)C5)nc3c2F)c2c(F)c(F)ccc2c1]

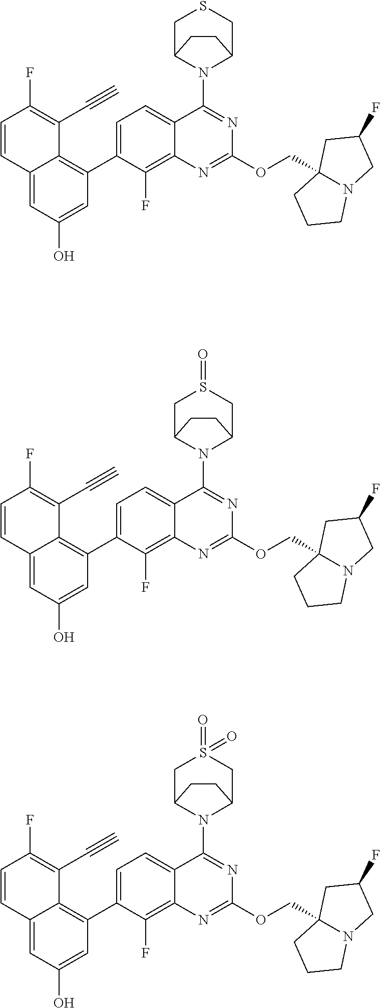 [SMILES: C#Cc1c(F)ccc2cc(O)cc(-c3ccc4c(N5C6CCC5CSC6)nc(OC[C@@]56CCCN5C[C@H](F)C6)nc4c3F)c12 ; C#Cc1c(F)ccc2cc(O)cc(-c3ccc4c(N5C6CCC5CS(=O)C6)nc(OC[C@@]56CCCN5C[C@H](F)C6)nc4c3F)c12 ; C#Cc1c(F)ccc2cc(O)cc(-c3ccc4c(N5C6CCC5CS(=O)(=O)C6)nc(OC[C@@]56CCCN5C[C@H](F)C6)nc4c3F)c12]

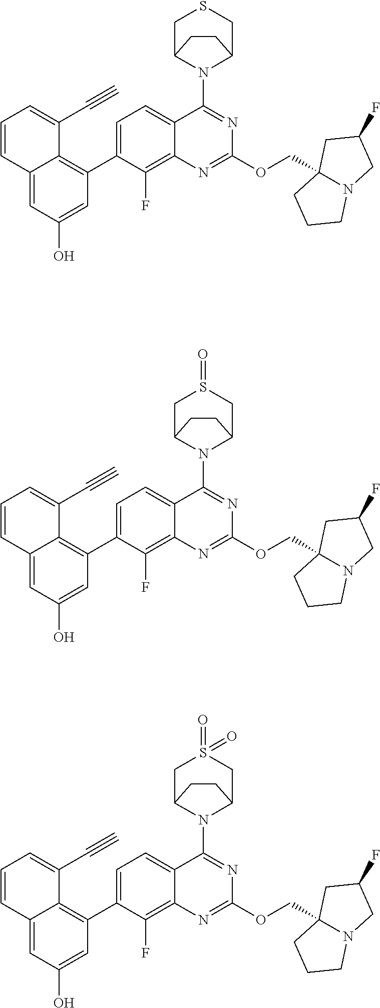 [SMILES: C#Cc1cccc2cc(O)cc(-c3ccc4c(N5C6CCC5CSC6)nc(OC[C@@]56CCCN5C[C@H](F)C6)nc4c3F)c12 ; C#Cc1cccc2cc(O)cc(-c3ccc4c(N5C6CCC5CS(=O)C6)nc(OC[C@@]56CCCN5C[C@H](F)C6)nc4c3F)c12 ; C#Cc1cccc2cc(O)cc(-c3ccc4c(N5C6CCC5CS(=O)(=O)C6)nc(OC[C@@]56CCCN5C[C@H](F)C6)nc4c3F)c12]

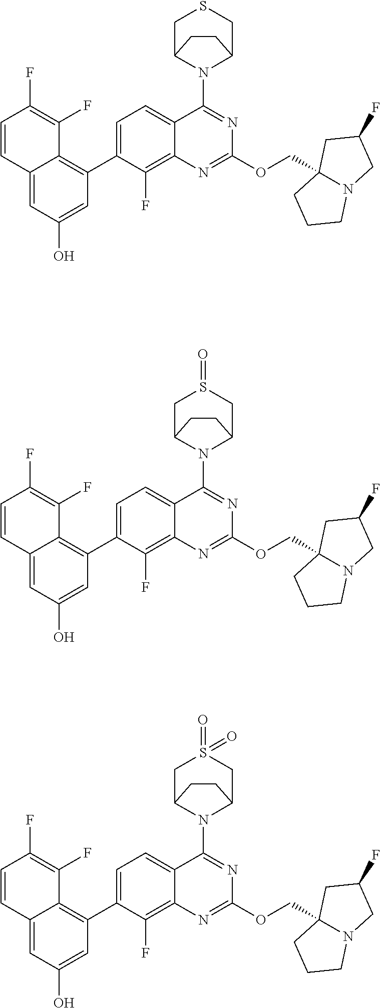 [SMILES: Oc1cc(-c2ccc3c(N4C5CCC4CSC5)nc(OC[C@@]45CCCN4C[C@H](F)C5)nc3c2F)c2c(F)c(F)ccc2c1 ; O=S1CC2CCC(C1)N2c1nc(OC[C@@]23CCCN2C[C@H](F)C3)nc2c(F)c(-c3cc(O)cc4ccc(F)c(F)c34)ccc12 ; O=S1(=O)CC2CCC(C1)N2c1nc(OC[C@@]23CCCN2C[C@H](F)C3)nc2c(F)c(-c3cc(O)cc4ccc(F)c(F)c34)ccc12]

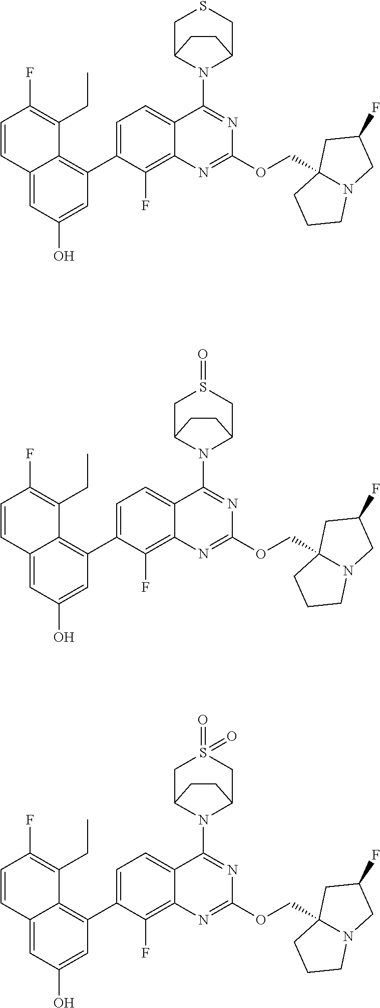 [SMILES: CCc1c(F)ccc2cc(O)cc(-c3ccc4c(N5C6CCC5CSC6)nc(OC[C@@]56CCCN5C[C@H](F)C6)nc4c3F)c12 ; CCc1c(F)ccc2cc(O)cc(-c3ccc4c(N5C6CCC5CS(=O)C6)nc(OC[C@@]56CCCN5C[C@H](F)C6)nc4c3F)c12 ; CCc1c(F)ccc2cc(O)cc(-c3ccc4c(N5C6CCC5CS(=O)(=O)C6)nc(OC[C@@]56CCCN5C[C@H](F)C6)nc4c3F)c12]

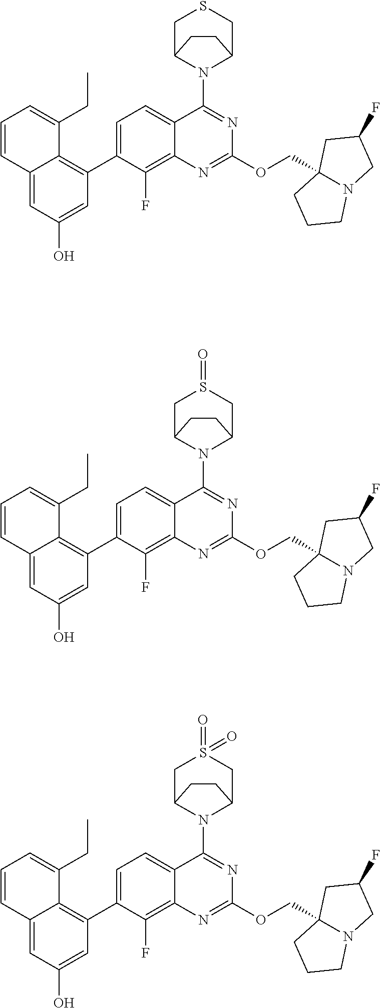 [SMILES: CCc1cccc2cc(O)cc(-c3ccc4c(N5C6CCC5CSC6)nc(OC[C@@]56CCCN5C[C@H](F)C6)nc4c3F)c12 ; CCc1cccc2cc(O)cc(-c3ccc4c(N5C6CCC5CS(=O)C6)nc(OC[C@@]56CCCN5C[C@H](F)C6)nc4c3F)c12 ; CCc1cccc2cc(O)cc(-c3ccc4c(N5C6CCC5CS(=O)(=O)C6)nc(OC[C@@]56CCCN5C[C@H](F)C6)nc4c3F)c12]

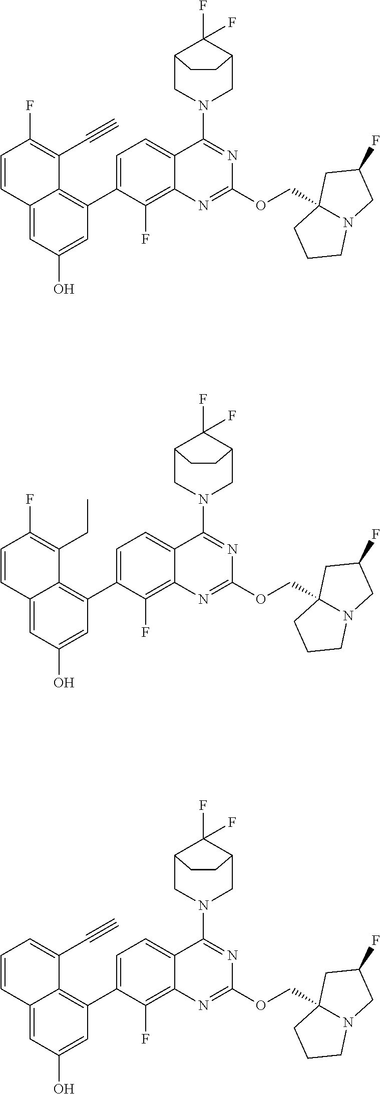 [SMILES: C#Cc1c(F)ccc2cc(O)cc(-c3ccc4c(N5CC6CCC(C5)C6(F)F)nc(OC[C@@]56CCCN5C[C@H](F)C6)nc4c3F)c12 ; CCc1c(F)ccc2cc(O)cc(-c3ccc4c(N5CC6CCC(C5)C6(F)F)nc(OC[C@@]56CCCN5C[C@H](F)C6)nc4c3F)c12 ; C#Cc1cccc2cc(O)cc(-c3ccc4c(N5CC6CCC(C5)C6(F)F)nc(OC[C@@]56CCCN5C[C@H](F)C6)nc4c3F)c12]

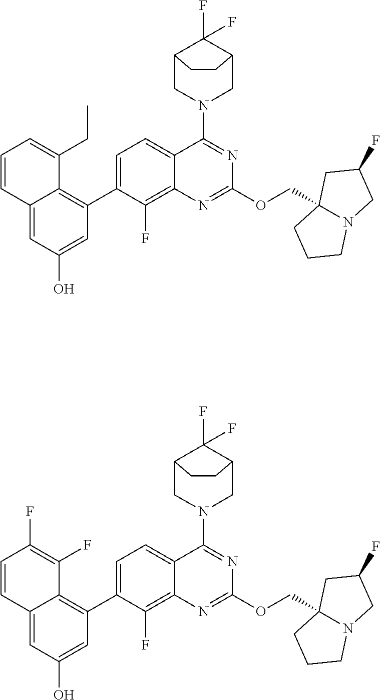 [SMILES: CCc1cccc2cc(O)cc(-c3ccc4c(N5CC6CCC(C5)C6(F)F)nc(OC[C@@]56CCCN5C[C@H](F)C6)nc4c3F)c12 ; Oc1cc(-c2ccc3c(N4CC5CCC(C4)C5(F)F)nc(OC[C@@]45CCCN4C[C@H](F)C5)nc3c2F)c2c(F)c(F)ccc2c1]

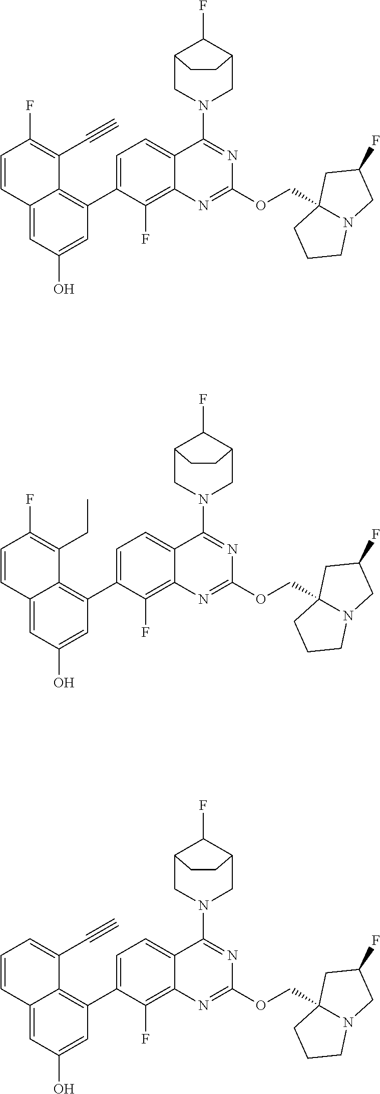 [SMILES: C#Cc1c(F)ccc2cc(O)cc(-c3ccc4c(N5CC6CCC(C5)C6F)nc(OC[C@@]56CCCN5C[C@H](F)C6)nc4c3F)c12 ; CCc1c(F)ccc2cc(O)cc(-c3ccc4c(N5CC6CCC(C5)C6F)nc(OC[C@@]56CCCN5C[C@H](F)C6)nc4c3F)c12 ; C#Cc1cccc2cc(O)cc(-c3ccc4c(N5CC6CCC(C5)C6F)nc(OC[C@@]56CCCN5C[C@H](F)C6)nc4c3F)c12]

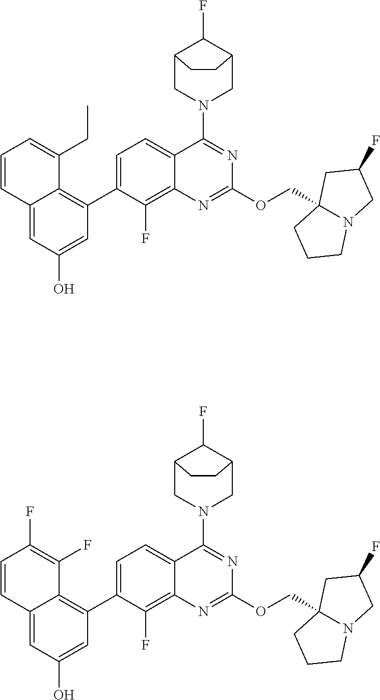 [SMILES: CCc1cccc2cc(O)cc(-c3ccc4c(N5CC6CCC(C5)C6F)nc(OC[C@@]56CCCN5C[C@H](F)C6)nc4c3F)c12 ; Oc1cc(-c2ccc3c(N4CC5CCC(C4)C5F)nc(OC[C@@]45CCCN4C[C@H](F)C5)nc3c2F)c2c(F)c(F)ccc2c1]

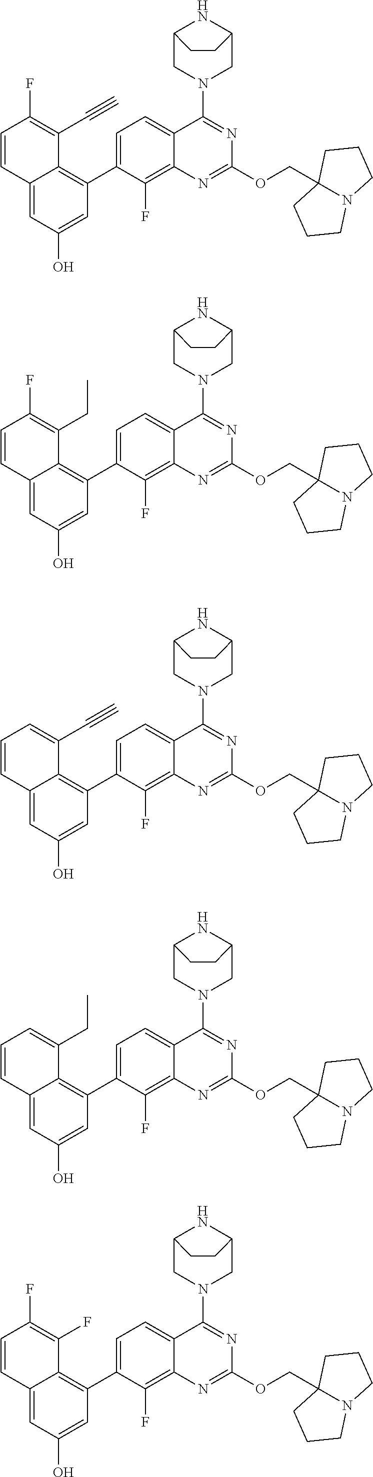 [SMILES: C#Cc1c(F)ccc2cc(O)cc(-c3ccc4c(N5CC6CCC(C5)N6)nc(OCC56CCCN5CCC6)nc4c3F)c12 ; CCc1c(F)ccc2cc(O)cc(-c3ccc4c(N5CC6CCC(C5)N6)nc(OCC56CCCN5CCC6)nc4c3F)c12 ; C#Cc1cccc2cc(O)cc(-c3ccc4c(N5CC6CCC(C5)N6)nc(OCC56CCCN5CCC6)nc4c3F)c12 ; CCc1cccc2cc(O)cc(-c3ccc4c(N5CC6CCC(C5)N6)nc(OCC56CCCN5CCC6)nc4c3F)c12 ; Oc1cc(-c2ccc3c(N4CC5CCC(C4)N5)nc(OCC45CCCN4CCC5)nc3c2F)c2c(F)c(F)ccc2c1]

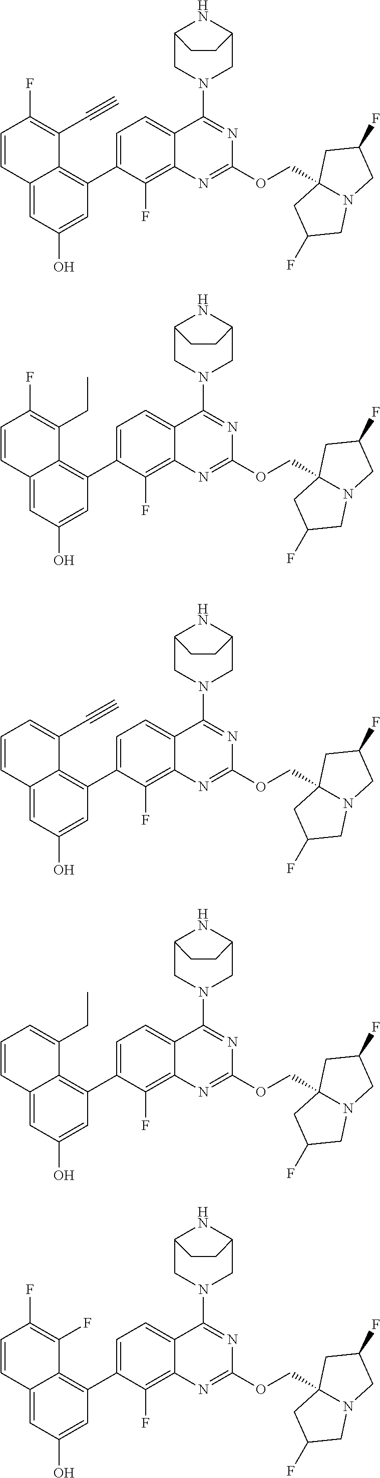 [SMILES: C#Cc1c(F)ccc2cc(O)cc(-c3ccc4c(N5CC6CCC(C5)N6)nc(OC[C@@]56CC(F)CN5C[C@H](F)C6)nc4c3F)c12 ; CCc1c(F)ccc2cc(O)cc(-c3ccc4c(N5CC6CCC(C5)N6)nc(OC[C@@]56CC(F)CN5C[C@H](F)C6)nc4c3F)c12 ; C#Cc1cccc2cc(O)cc(-c3ccc4c(N5CC6CCC(C5)N6)nc(OC[C@@]56CC(F)CN5C[C@H](F)C6)nc4c3F)c12 ; CCc1cccc2cc(O)cc(-c3ccc4c(N5CC6CCC(C5)N6)nc(OC[C@@]56CC(F)CN5C[C@H](F)C6)nc4c3F)c12 ; Oc1cc(-c2ccc3c(N4CC5CCC(C4)N5)nc(OC[C@@]45CC(F)CN4C[C@H](F)C5)nc3c2F)c2c(F)c(F)ccc2c1]

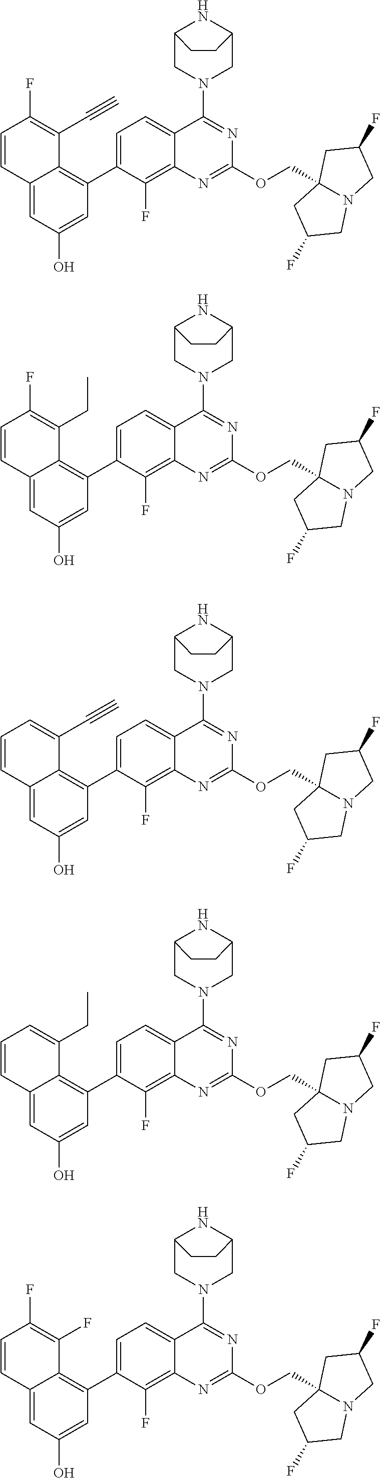

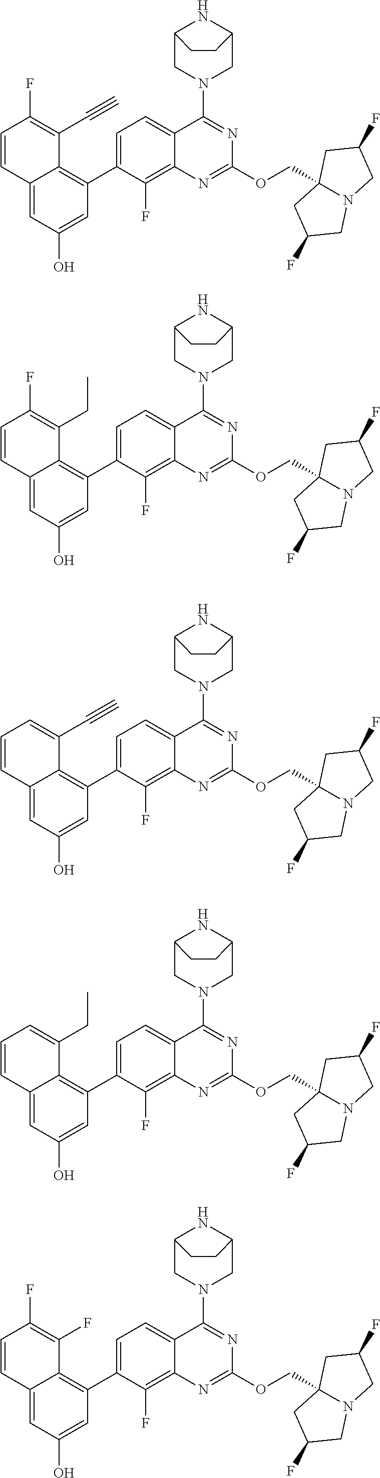 [SMILES: C#Cc1c(F)ccc2cc(O)cc(-c3ccc4c(N5CC6CCC(C5)N6)nc(OC[C@@]56C[C@H](F)CN5C[C@H](F)C6)nc4c3F)c12 ; CCc1c(F)ccc2cc(O)cc(-c3ccc4c(N5CC6CCC(C5)N6)nc(OC[C@@]56C[C@H](F)CN5C[C@H](F)C6)nc4c3F)c12 ; C#Cc1cccc2cc(O)cc(-c3ccc4c(N5CC6CCC(C5)N6)nc(OC[C@@]56C[C@H](F)CN5C[C@H](F)C6)nc4c3F)c12 ; CCc1cccc2cc(O)cc(-c3ccc4c(N5CC6CCC(C5)N6)nc(OC[C@@]56C[C@H](F)CN5C[C@H](F)C6)nc4c3F)c12 ; Oc1cc(-c2ccc3c(N4CC5CCC(C4)N5)nc(OC[C@@]45C[C@H](F)CN4C[C@H](F)C5)nc3c2F)c2c(F)c(F)ccc2c1]

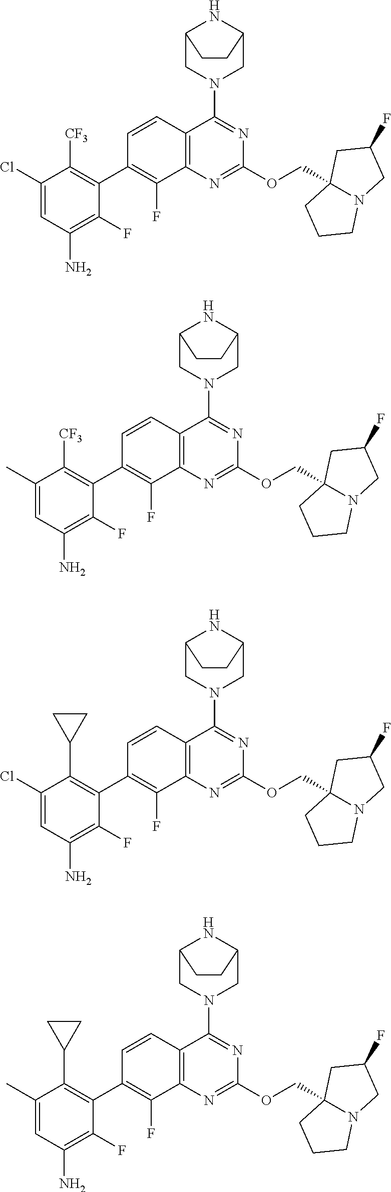 [SMILES: Nc1cc(Cl)c(C(F)(F)F)c(-c2ccc3c(N4CC5CCC(C4)N5)nc(OC[C@@]45CCCN4C[C@H](F)C5)nc3c2F)c1F ; Cc1cc(N)c(F)c(-c2ccc3c(N4CC5CCC(C4)N5)nc(OC[C@@]45CCCN4C[C@H](F)C5)nc3c2F)c1C(F)(F)F ; Nc1cc(Cl)c(C2CC2)c(-c2ccc3c(N4CC5CCC(C4)N5)nc(OC[C@@]45CCCN4C[C@H](F)C5)nc3c2F)c1F ; Cc1cc(N)c(F)c(-c2ccc3c(N4CC5CCC(C4)N5)nc(OC[C@@]45CCCN4C[C@H](F)C5)nc3c2F)c1C1CC1]

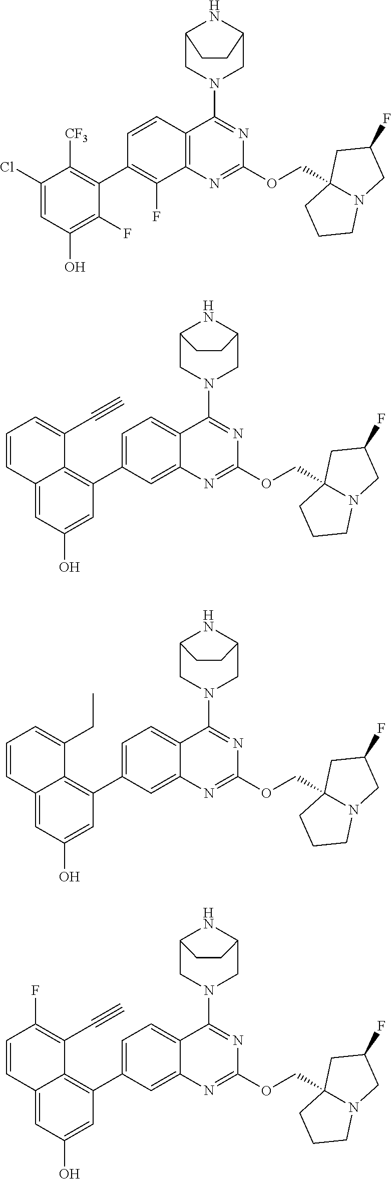 [SMILES: Oc1cc(Cl)c(C(F)(F)F)c(-c2ccc3c(N4CC5CCC(C4)N5)nc(OC[C@@]45CCCN4C[C@H](F)C5)nc3c2F)c1F ; C#Cc1cccc2cc(O)cc(-c3ccc4c(N5CC6CCC(C5)N6)nc(OC[C@@]56CCCN5C[C@H](F)C6)nc4c3)c12 ; CCc1cccc2cc(O)cc(-c3ccc4c(N5CC6CCC(C5)N6)nc(OC[C@@]56CCCN5C[C@H](F)C6)nc4c3)c12 ; C#Cc1c(F)ccc2cc(O)cc(-c3ccc4c(N5CC6CCC(C5)N6)nc(OC[C@@]56CCCN5C[C@H](F)C6)nc4c3)c12]

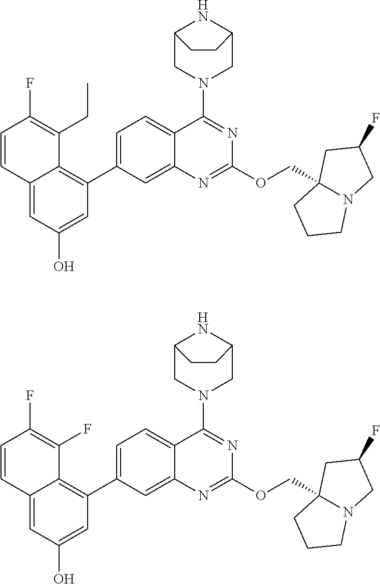 [SMILES: CCc1c(F)ccc2cc(O)cc(-c3ccc4c(N5CC6CCC(C5)N6)nc(OC[C@@]56CCCN5C[C@H](F)C6)nc4c3)c12 ; Oc1cc(-c2ccc3c(N4CC5CCC(C4)N5)nc(OC[C@@]45CCCN4C[C@H](F)C5)nc3c2)c2c(F)c(F)ccc2c1]

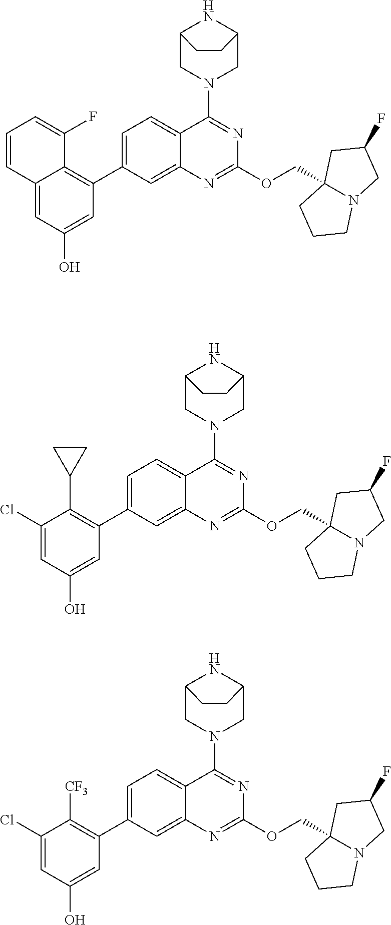 [SMILES: Oc1cc(-c2ccc3c(N4CC5CCC(C4)N5)nc(OC[C@@]45CCCN4C[C@H](F)C5)nc3c2)c2c(F)cccc2c1 ; Oc1cc(Cl)c(C2CC2)c(-c2ccc3c(N4CC5CCC(C4)N5)nc(OC[C@@]45CCCN4C[C@H](F)C5)nc3c2)c1 ; Oc1cc(Cl)c(C(F)(F)F)c(-c2ccc3c(N4CC5CCC(C4)N5)nc(OC[C@@]45CCCN4C[C@H](F)C5)nc3c2)c1]

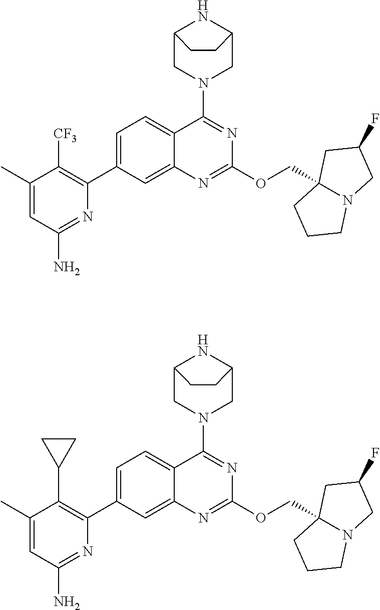 [SMILES: Cc1cc(N)nc(-c2ccc3c(N4CC5CCC(C4)N5)nc(OC[C@@]45CCCN4C[C@H](F)C5)nc3c2)c1C(F)(F)F ; Cc1cc(N)nc(-c2ccc3c(N4CC5CCC(C4)N5)nc(OC[C@@]45CCCN4C[C@H](F)C5)nc3c2)c1C1CC1]

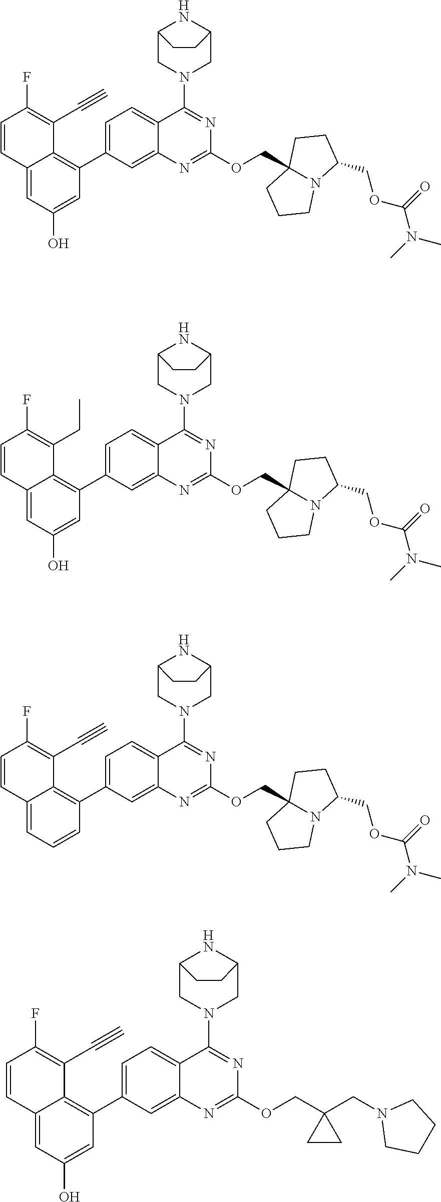 [SMILES: C#Cc1c(F)ccc2cc(O)cc(-c3ccc4c(N5CC6CCC(C5)N6)nc(OC[C@]56CCCN5[C@@H](COC(=O)N(C)C)CC6)nc4c3)c12 ; CCc1c(F)ccc2cc(O)cc(-c3ccc4c(N5CC6CCC(C5)N6)nc(OC[C@]56CCCN5[C@@H](COC(=O)N(C)C)CC6)nc4c3)c12 ; C#Cc1c(F)ccc2cccc(-c3ccc4c(N5CC6CCC(C5)N6)nc(OC[C@]56CCCN5[C@@H](COC(=O)N(C)C)CC6)nc4c3)c12 ; C#Cc1c(F)ccc2cc(O)cc(-c3ccc4c(N5CC6CCC(C5)N6)nc(OCC5(CN6CCCC6)CC5)nc4c3)c12]

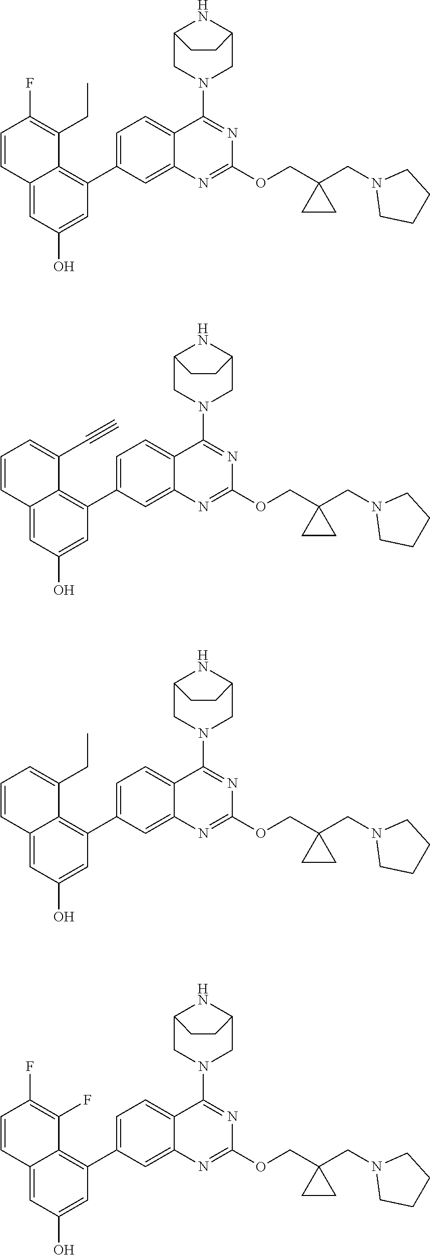 [SMILES: CCc1c(F)ccc2cc(O)cc(-c3ccc4c(N5CC6CCC(C5)N6)nc(OCC5(CN6CCCC6)CC5)nc4c3)c12 ; C#Cc1cccc2cc(O)cc(-c3ccc4c(N5CC6CCC(C5)N6)nc(OCC5(CN6CCCC6)CC5)nc4c3)c12 ; CCc1cccc2cc(O)cc(-c3ccc4c(N5CC6CCC(C5)N6)nc(OCC5(CN6CCCC6)CC5)nc4c3)c12 ; Oc1cc(-c2ccc3c(N4CC5CCC(C4)N5)nc(OCC4(CN5CCCC5)CC4)nc3c2)c2c(F)c(F)ccc2c1]

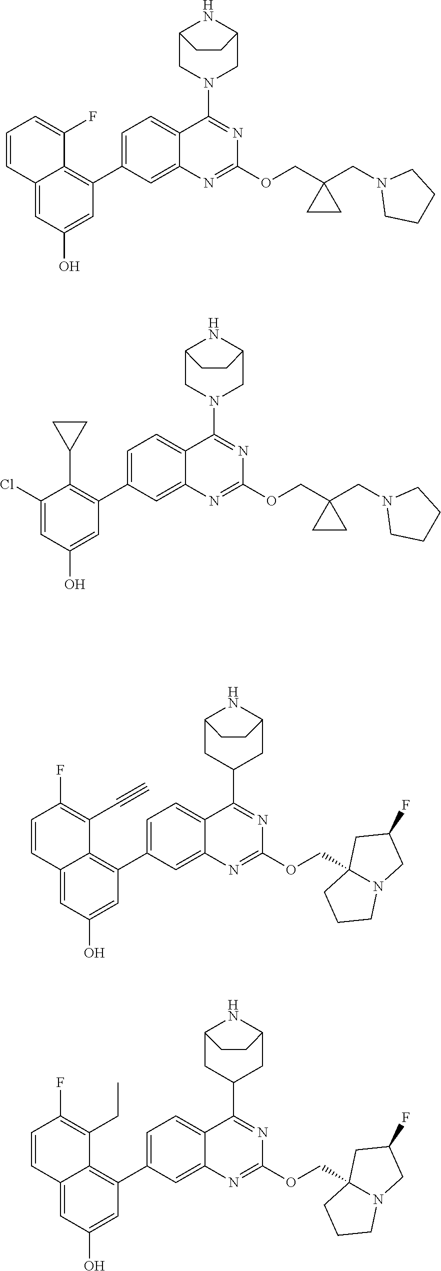 [SMILES: Oc1cc(-c2ccc3c(N4CC5CCC(C4)N5)nc(OCC4(CN5CCCC5)CC4)nc3c2)c2c(F)cccc2c1 ; Oc1cc(Cl)c(C2CC2)c(-c2ccc3c(N4CC5CCC(C4)N5)nc(OCC4(CN5CCCC5)CC4)nc3c2)c1 ; C#Cc1c(F)ccc2cc(O)cc(-c3ccc4c(C5CC6CCC(C5)N6)nc(OC[C@@]56CCCN5C[C@H](F)C6)nc4c3)c12 ; CCc1c(F)ccc2cc(O)cc(-c3ccc4c(C5CC6CCC(C5)N6)nc(OC[C@@]56CCCN5C[C@H](F)C6)nc4c3)c12]

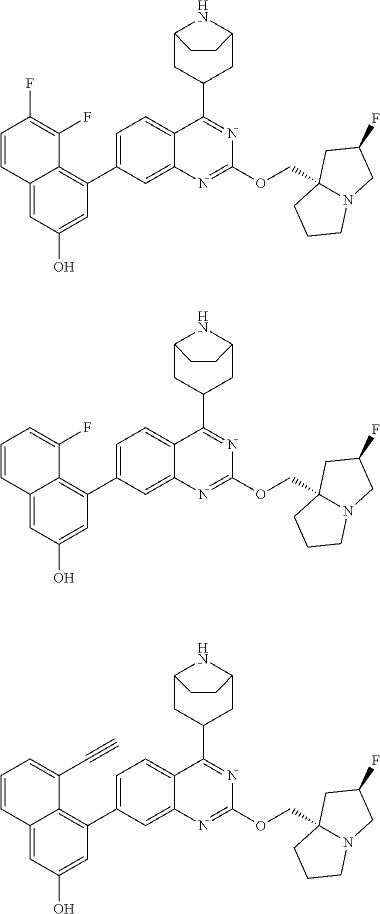 [SMILES: Oc1cc(-c2ccc3c(C4CC5CCC(C4)N5)nc(OC[C@@]45CCCN4C[C@H](F)C5)nc3c2)c2c(F)c(F)ccc2c1 ; Oc1cc(-c2ccc3c(C4CC5CCC(C4)N5)nc(OC[C@@]45CCCN4C[C@H](F)C5)nc3c2)c2c(F)cccc2c1 ; C#Cc1cccc2cc(O)cc(-c3ccc4c(C5CC6CCC(C5)N6)nc(OC[C@@]56CCCN5C[C@H](F)C6)nc4c3)c12]

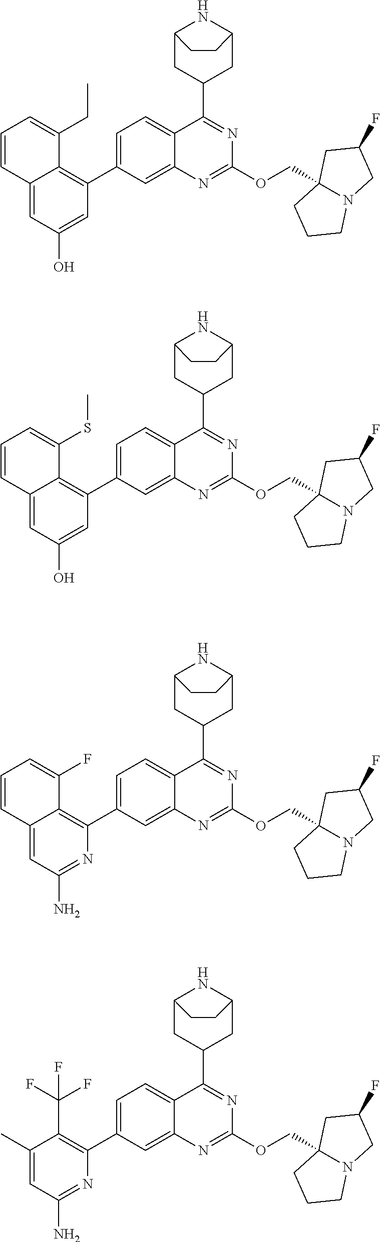 [SMILES: CCc1cccc2cc(O)cc(-c3ccc4c(C5CC6CCC(C5)N6)nc(OC[C@@]56CCCN5C[C@H](F)C6)nc4c3)c12 ; CSc1cccc2cc(O)cc(-c3ccc4c(C5CC6CCC(C5)N6)nc(OC[C@@]56CCCN5C[C@H](F)C6)nc4c3)c12 ; Nc1cc2cccc(F)c2c(-c2ccc3c(C4CC5CCC(C4)N5)nc(OC[C@@]45CCCN4C[C@H](F)C5)nc3c2)n1 ; Cc1cc(N)nc(-c2ccc3c(C4CC5CCC(C4)N5)nc(OC[C@@]45CCCN4C[C@H](F)C5)nc3c2)c1C(F)(F)F]

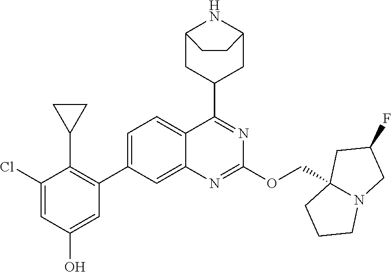 [SMILES: Oc1cc(Cl)c(C2CC2)c(-c2ccc3c(C4CC5CCC(C4)N5)nc(OC[C@@]45CCCN4C[C@H](F)C5)nc3c2)c1]

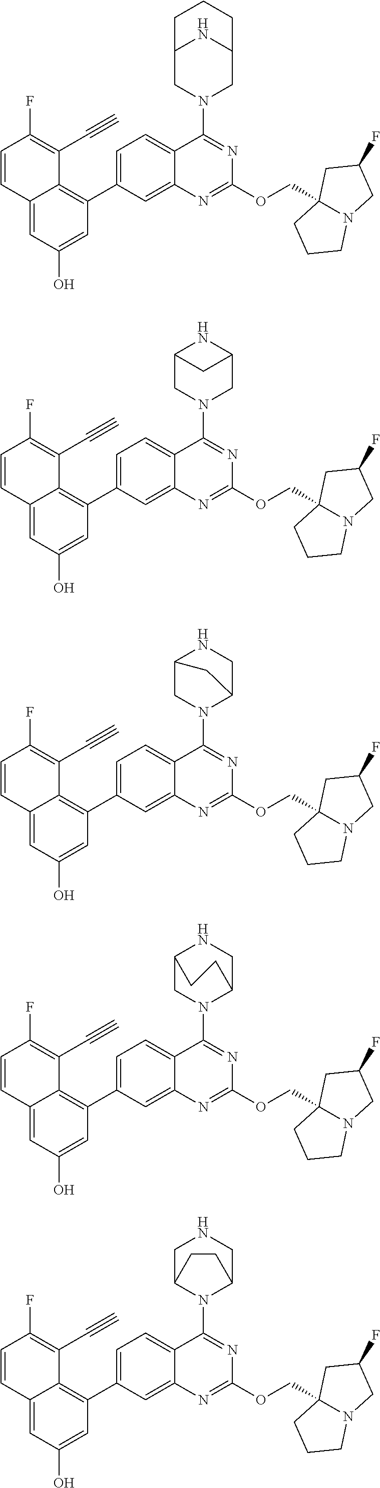 [SMILES: C#Cc1c(F)ccc2cc(O)cc(-c3ccc4c(N5CC6CCCC(C5)N6)nc(OC[C@@]56CCCN5C[C@H](F)C6)nc4c3)c12 ; C#Cc1c(F)ccc2cc(O)cc(-c3ccc4c(N5CC6CC(C5)N6)nc(OC[C@@]56CCCN5C[C@H](F)C6)nc4c3)c12 ; C#Cc1c(F)ccc2cc(O)cc(-c3ccc4c(N5CC6CC5CN6)nc(OC[C@@]56CCCN5C[C@H](F)C6)nc4c3)c12 ; C#Cc1c(F)ccc2cc(O)cc(-c3ccc4c(N5CC6CCC5CN6)nc(OC[C@@]56CCCN5C[C@H](F)C6)nc4c3)c12 ; C#Cc1c(F)ccc2cc(O)cc(-c3ccc4c(N5C6CCC5CNC6)nc(OC[C@@]56CCCN5C[C@H](F)C6)nc4c3)c12]

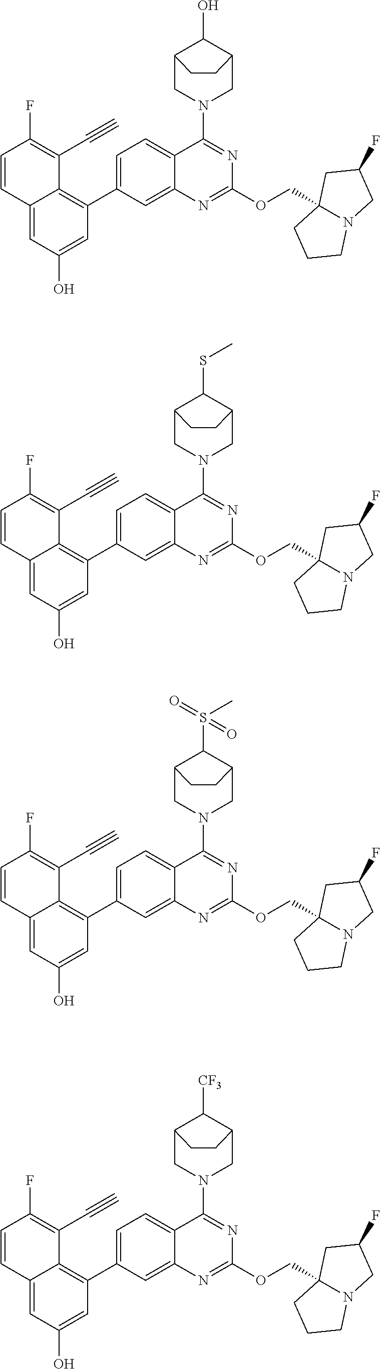 [SMILES: C#Cc1c(F)ccc2cc(O)cc(-c3ccc4c(N5CC6CCC(C5)C6O)nc(OC[C@@]56CCCN5C[C@H](F)C6)nc4c3)c12 ; C#Cc1c(F)ccc2cc(O)cc(-c3ccc4c(N5CC6CCC(C5)C6SC)nc(OC[C@@]56CCCN5C[C@H](F)C6)nc4c3)c12 ; C#Cc1c(F)ccc2cc(O)cc(-c3ccc4c(N5CC6CCC(C5)C6S(C)(=O)=O)nc(OC[C@@]56CCCN5C[C@H](F)C6)nc4c3)c12 ; C#Cc1c(F)ccc2cc(O)cc(-c3ccc4c(N5CC6CCC(C5)C6C(F)(F)F)nc(OC[C@@]56CCCN5C[C@H](F)C6)nc4c3)c12]

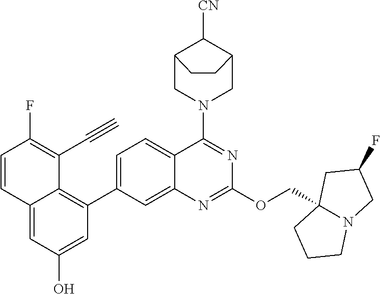 [SMILES: C#Cc1c(F)ccc2cc(O)cc(-c3ccc4c(N5CC6CCC(C5)C6C#N)nc(OC[C@@]56CCCN5C[C@H](F)C6)nc4c3)c12]

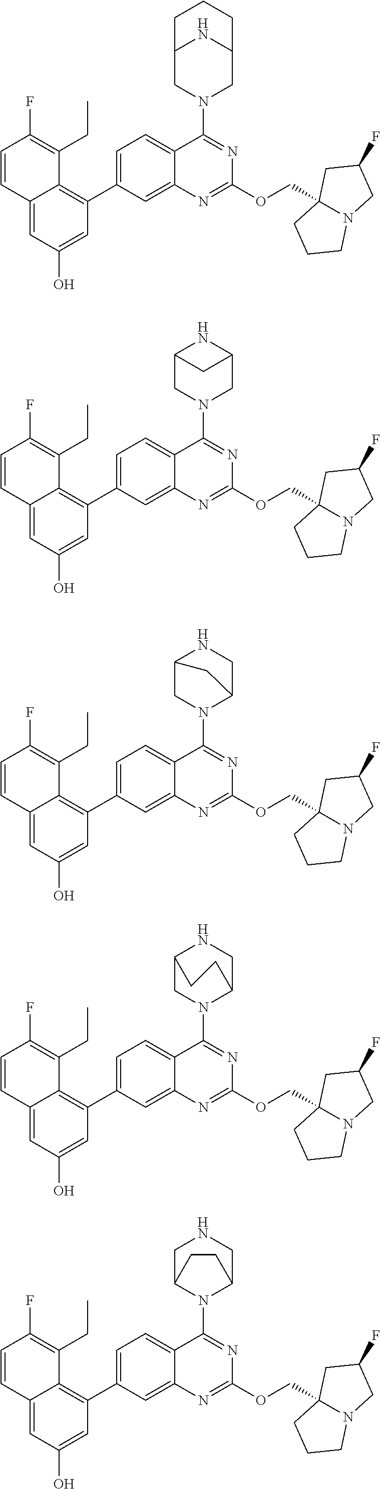 [SMILES: CCc1c(F)ccc2cc(O)cc(-c3ccc4c(N5CC6CCCC(C5)N6)nc(OC[C@@]56CCCN5C[C@H](F)C6)nc4c3)c12 ; CCc1c(F)ccc2cc(O)cc(-c3ccc4c(N5CC6CC(C5)N6)nc(OC[C@@]56CCCN5C[C@H](F)C6)nc4c3)c12 ; CCc1c(F)ccc2cc(O)cc(-c3ccc4c(N5CC6CC5CN6)nc(OC[C@@]56CCCN5C[C@H](F)C6)nc4c3)c12 ; CCc1c(F)ccc2cc(O)cc(-c3ccc4c(N5CC6CCC5CN6)nc(OC[C@@]56CCCN5C[C@H](F)C6)nc4c3)c12 ; CCc1c(F)ccc2cc(O)cc(-c3ccc4c(N5C6CCC5CNC6)nc(OC[C@@]56CCCN5C[C@H](F)C6)nc4c3)c12]

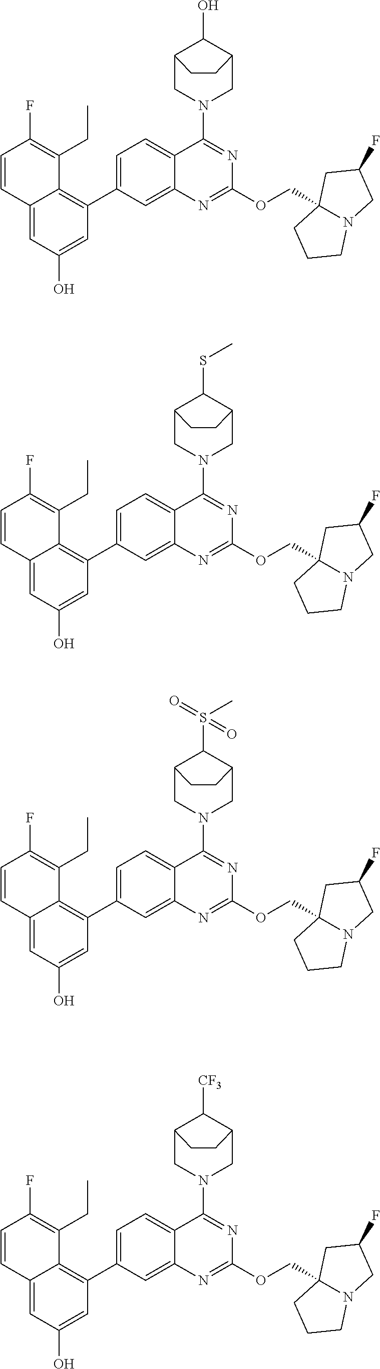 [SMILES: CCc1c(F)ccc2cc(O)cc(-c3ccc4c(N5CC6CCC(C5)C6O)nc(OC[C@@]56CCCN5C[C@H](F)C6)nc4c3)c12 ; CCc1c(F)ccc2cc(O)cc(-c3ccc4c(N5CC6CCC(C5)C6SC)nc(OC[C@@]56CCCN5C[C@H](F)C6)nc4c3)c12 ; CCc1c(F)ccc2cc(O)cc(-c3ccc4c(N5CC6CCC(C5)C6S(C)(=O)=O)nc(OC[C@@]56CCCN5C[C@H](F)C6)nc4c3)c12 ; CCc1c(F)ccc2cc(O)cc(-c3ccc4c(N5CC6CCC(C5)C6C(F)(F)F)nc(OC[C@@]56CCCN5C[C@H](F)C6)nc4c3)c12]

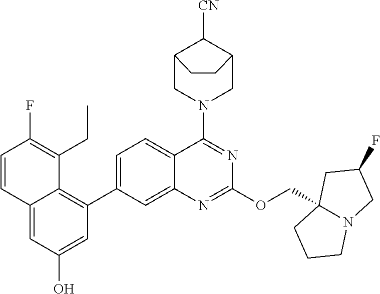 [SMILES: CCc1c(F)ccc2cc(O)cc(-c3ccc4c(N5CC6CCC(C5)C6C#N)nc(OC[C@@]56CCCN5C[C@H](F)C6)nc4c3)c12]

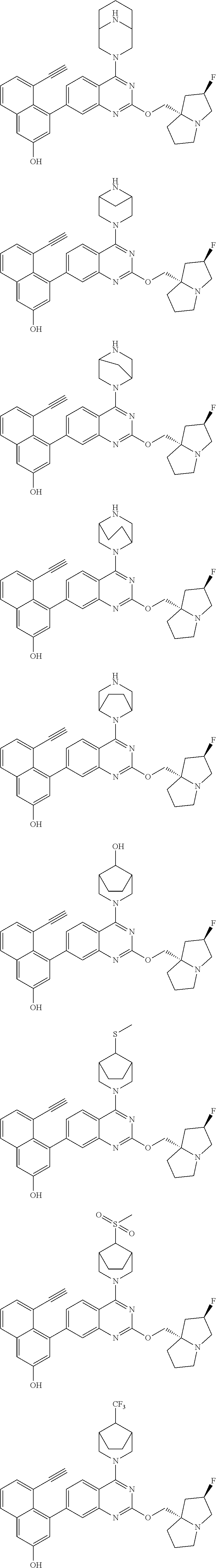

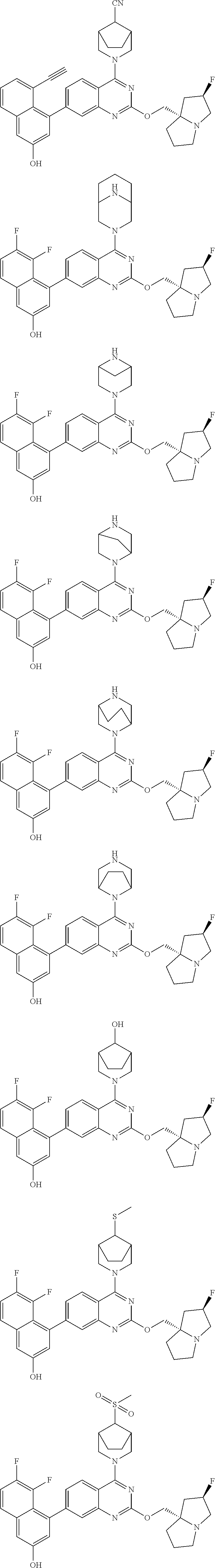

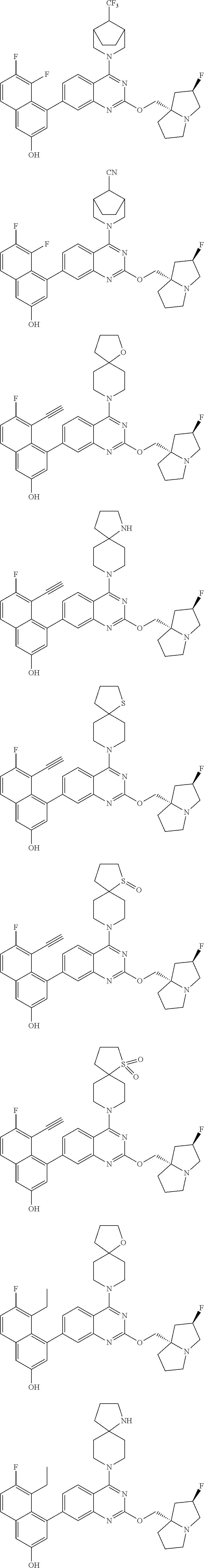

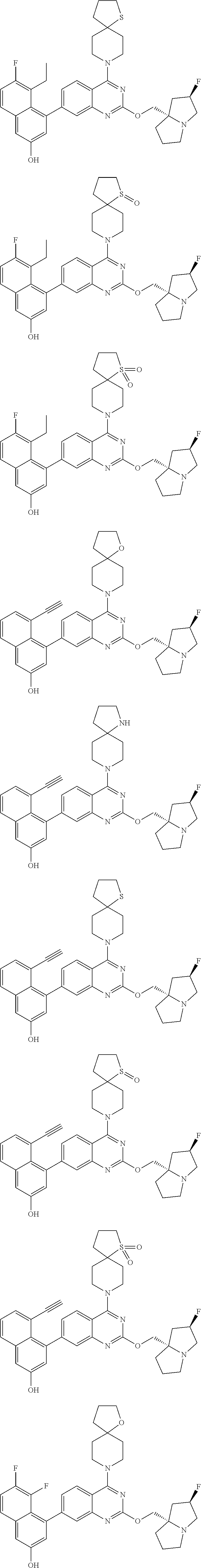

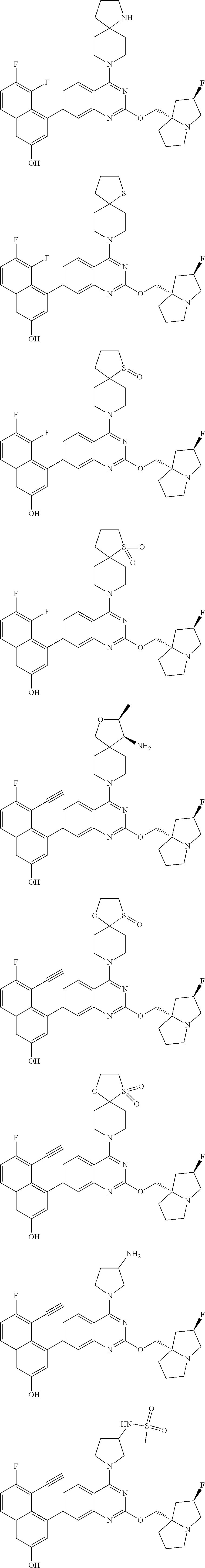

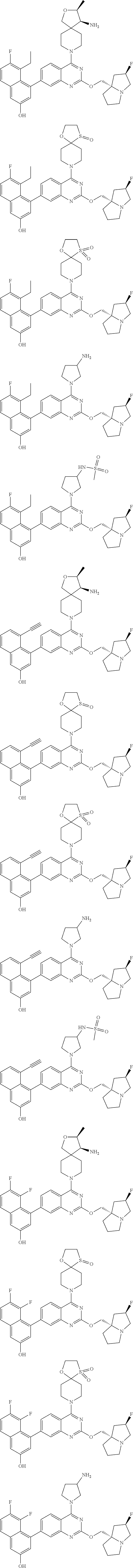

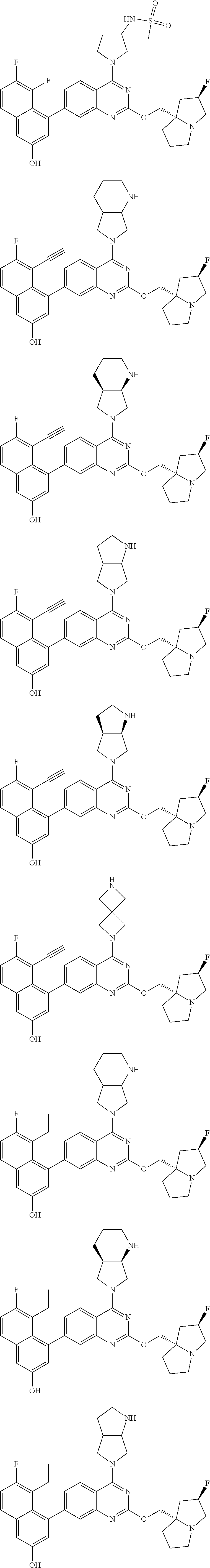

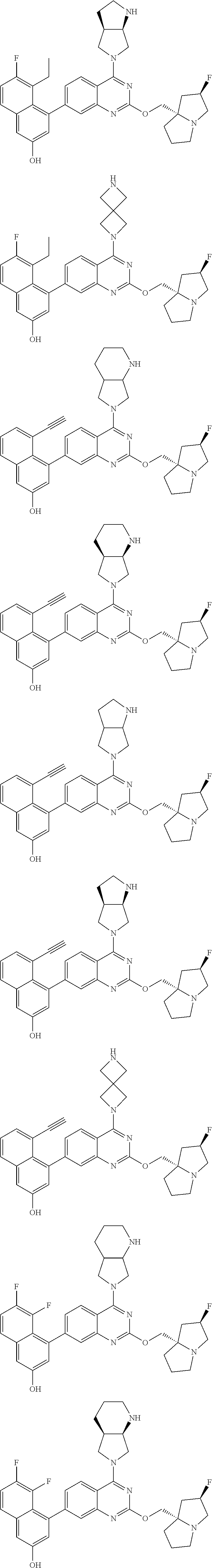

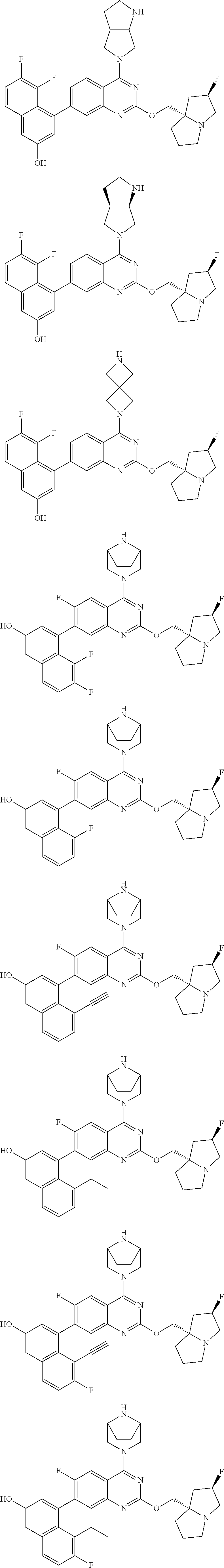

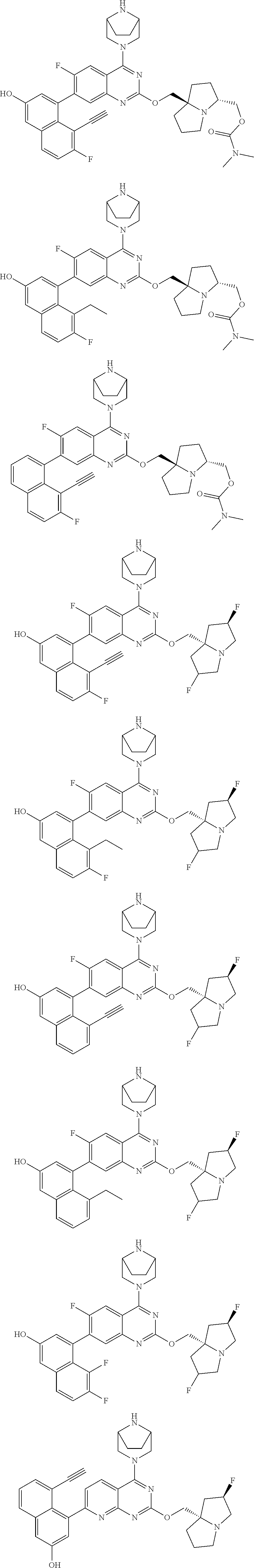

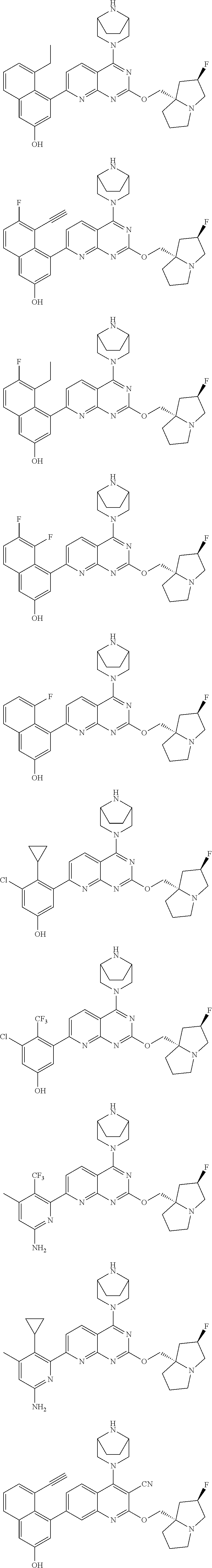

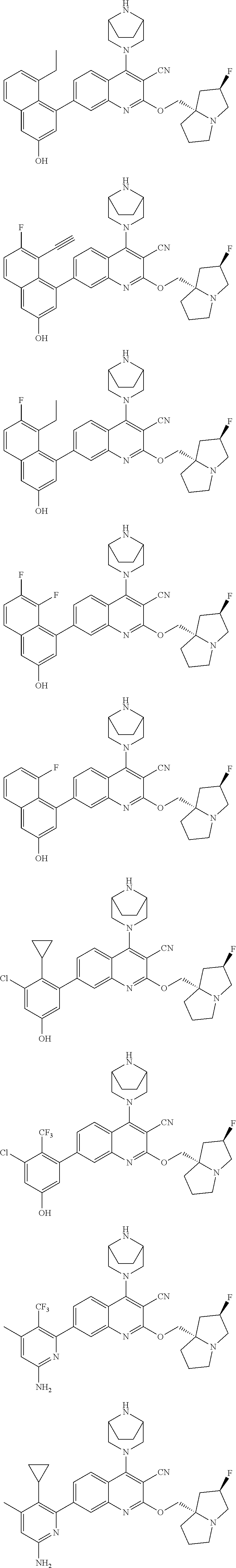

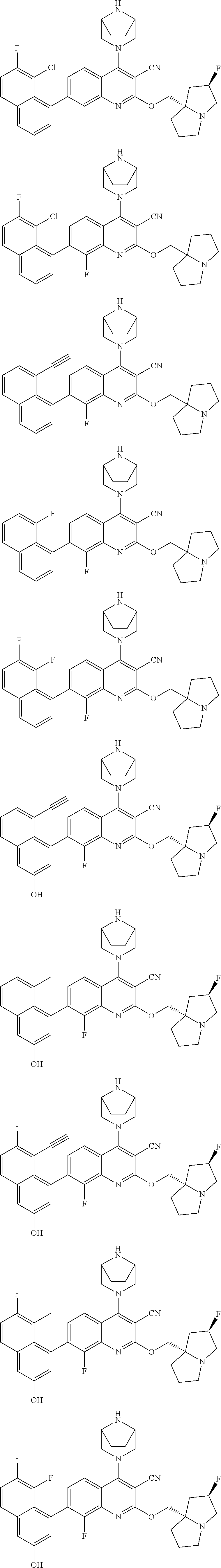

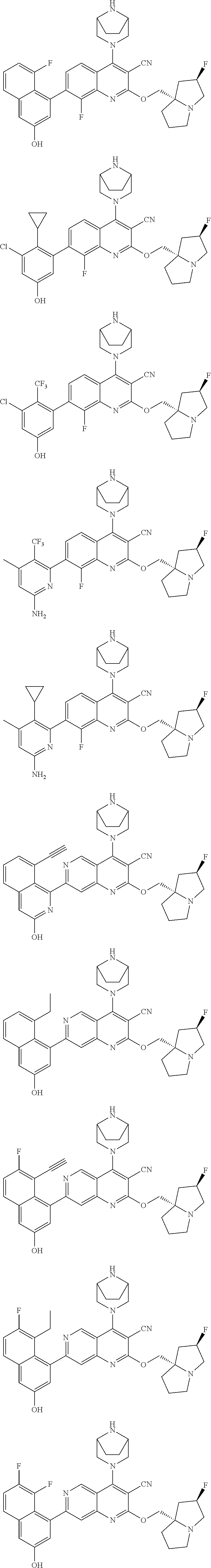

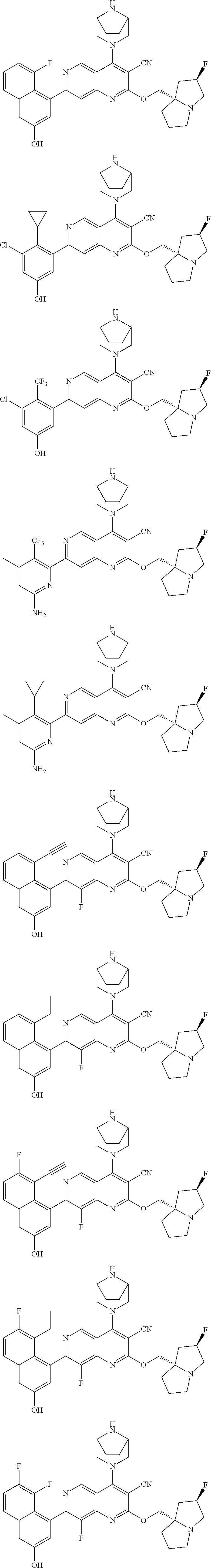

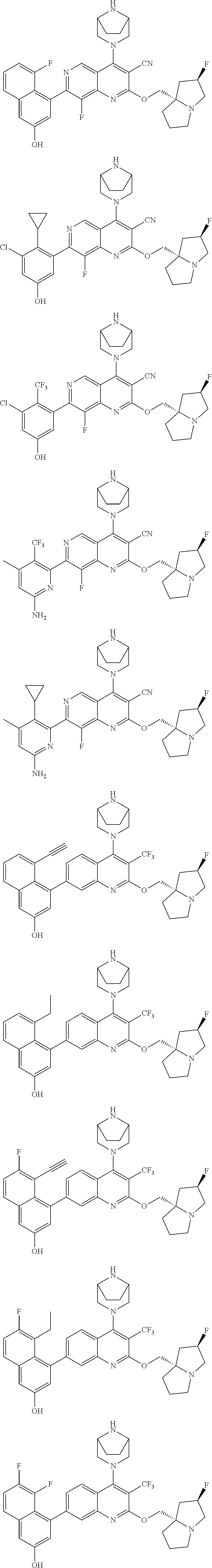

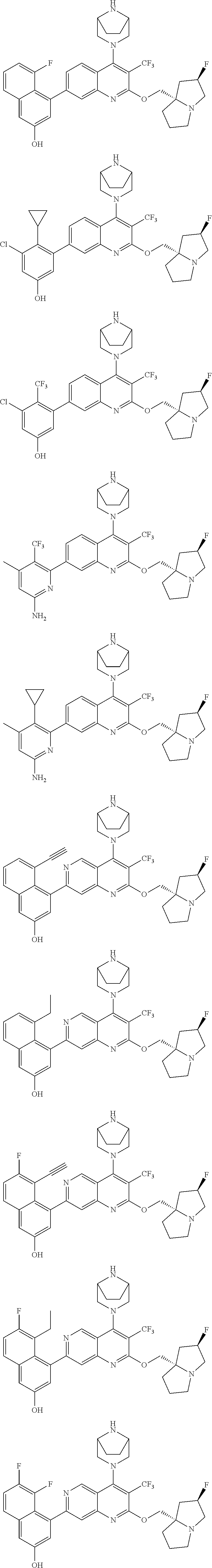

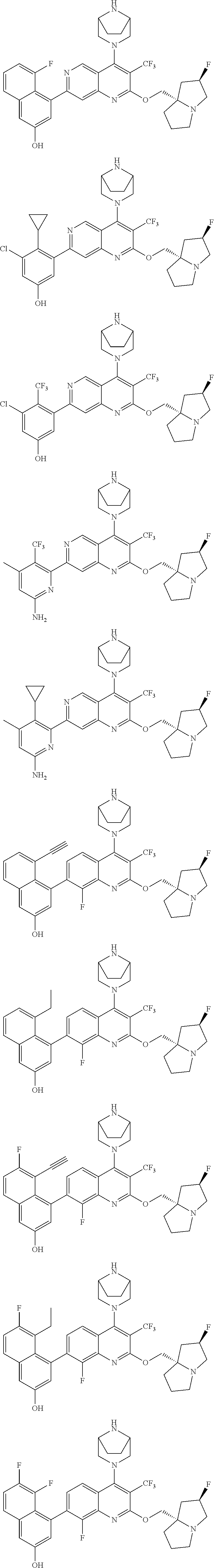

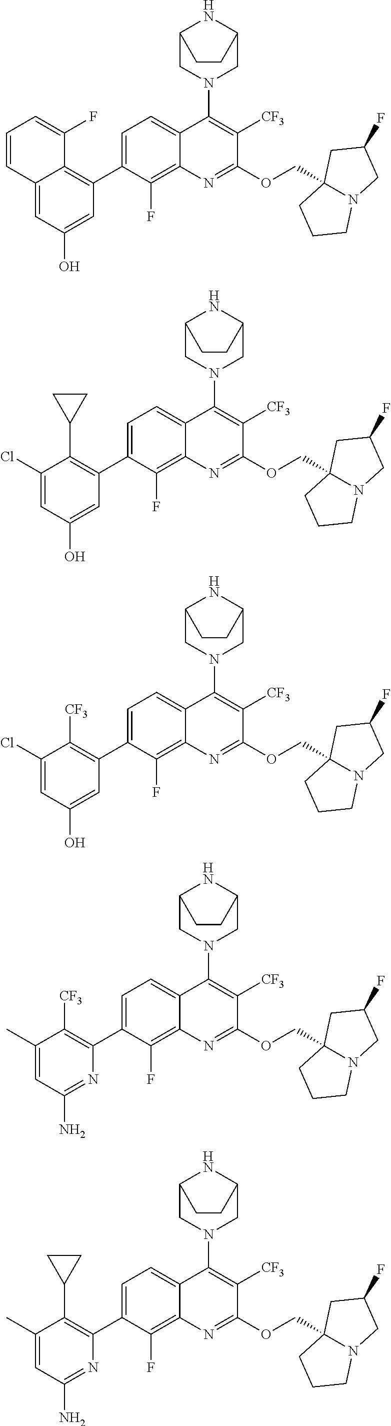 [SMILES: Oc1cc(-c2ccc3c(N4CC5CCC(C4)N5)c(C(F)(F)F)c(OC[C@@]45CCCN4C[C@H](F)C5)nc3c2F)c2c(F)cccc2c1 ; Oc1cc(Cl)c(C2CC2)c(-c2ccc3c(N4CC5CCC(C4)N5)c(C(F)(F)F)c(OC[C@@]45CCCN4C[C@H](F)C5)nc3c2F)c1 ; Oc1cc(Cl)c(C(F)(F)F)c(-c2ccc3c(N4CC5CCC(C4)N5)c(C(F)(F)F)c(OC[C@@]45CCCN4C[C@H](F)C5)nc3c2F)c1 ; Cc1cc(N)nc(-c2ccc3c(N4CC5CCC(C4)N5)c(C(F)(F)F)c(OC[C@@]45CCCN4C[C@H](F)C5)nc3c2F)c1C(F)(F)F ; Cc1cc(N)nc(-c2ccc3c(N4CC5CCC(C4)N5)c(C(F)(F)F)c(OC[C@@]45CCCN4C[C@H](F)C5)nc3c2F)c1C1CC1]

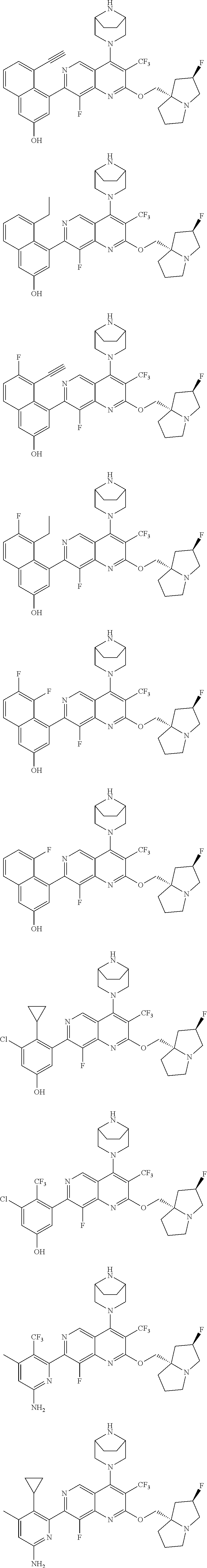

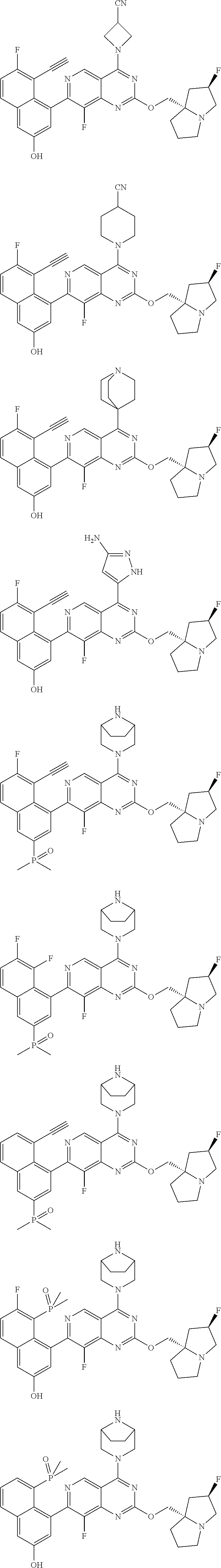

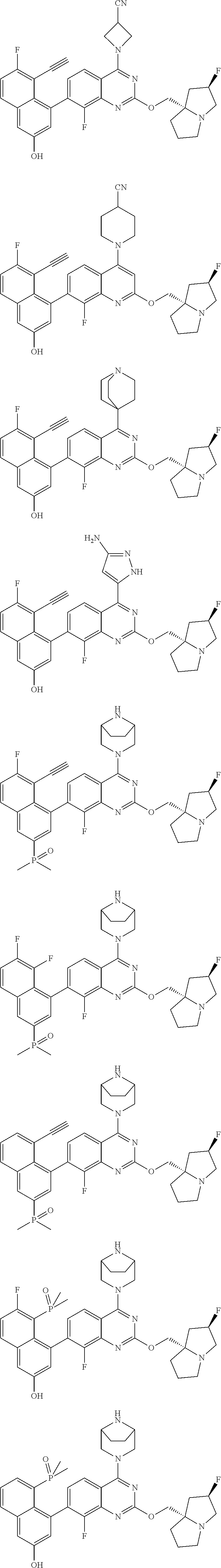

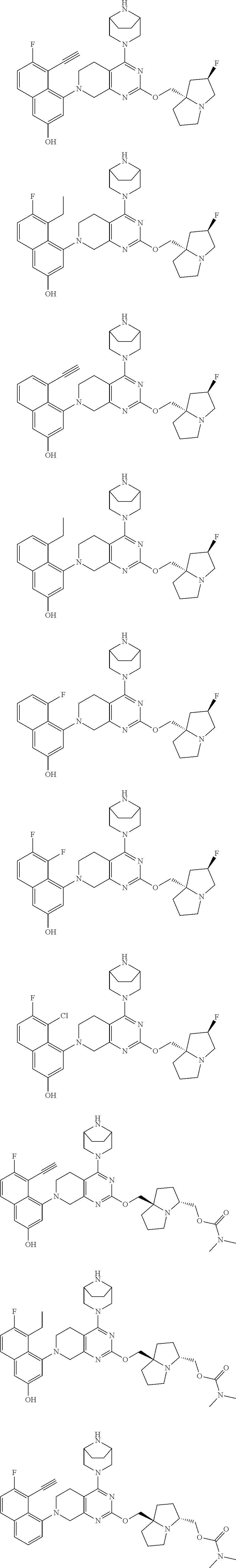

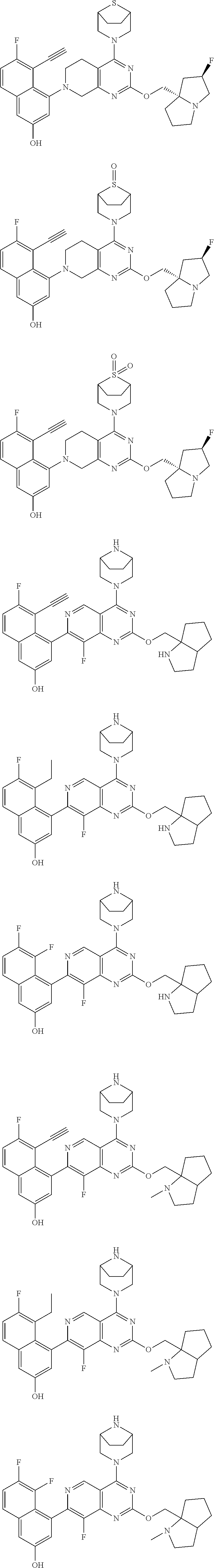

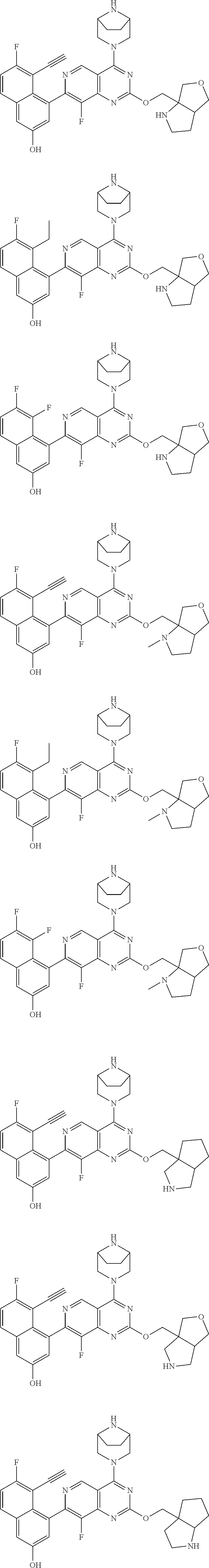

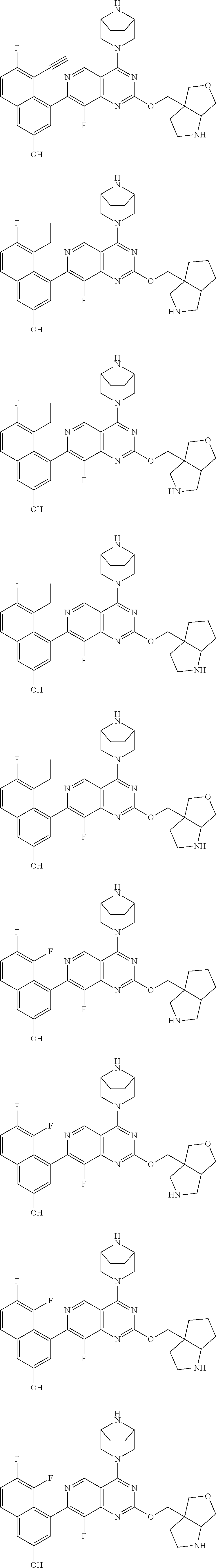

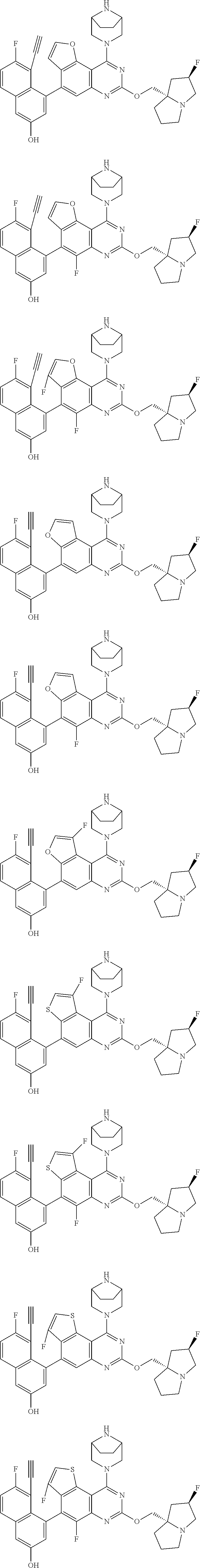

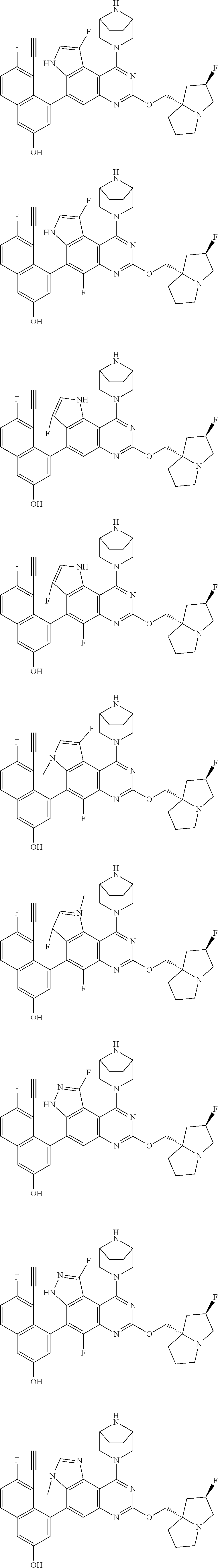

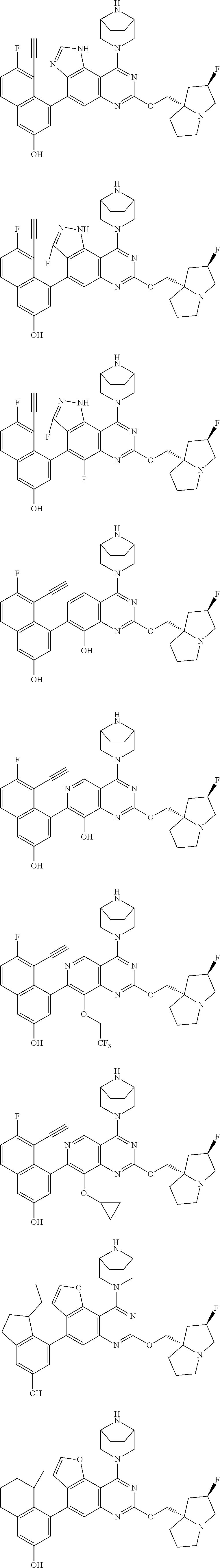

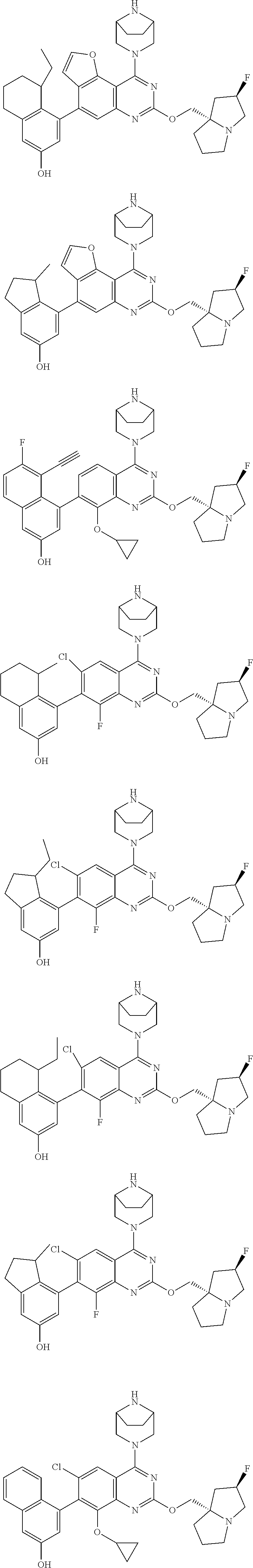

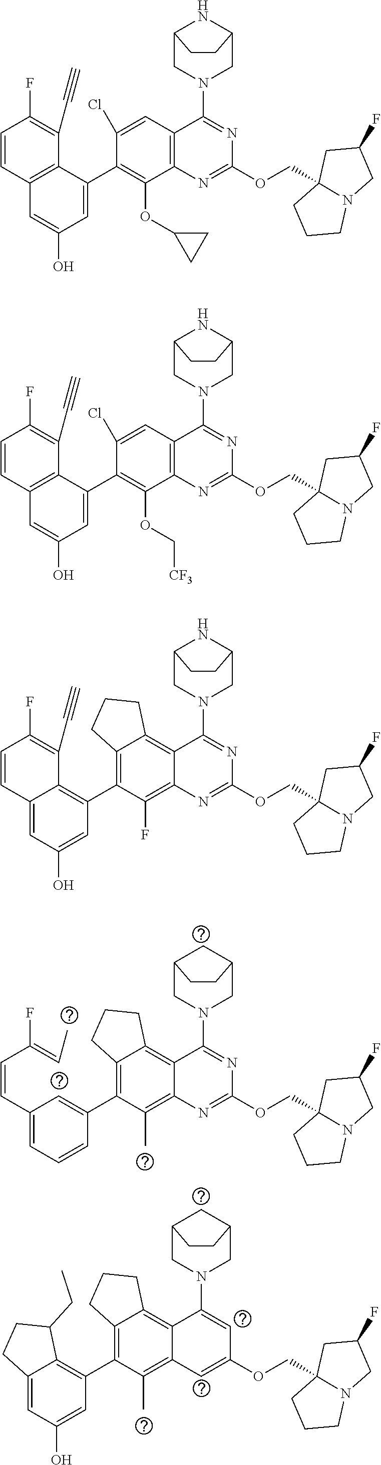 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3OC3CC3)c12 ; C#Cc1c(F)ccc2cc(O)cc(-c3c(Cl)cc4c(N5CC6CCC(C5)N6)nc(OC[C@@]56CCCN5C[C@H](F)C6)nc4c3OCC(F)(F)F)c12 ; C#Cc1c(F)ccc2cc(O)cc(-c3c4c(c5c(N6CC7CCC(C6)N7)nc(OC[C@@]67CCCN6C[C@H](F)C7)nc5c3F)CCC4)c12 ; C/C=C(F)/C=C\c1cccc(-c2c3c(c4c(N5CC6CCC(C6)C5)nc(OC[C@@]56CCCN5C[C@H](F)C6)nc4c2C)CCC3)c1 ; CCC1CCc2cc(O)cc(-c3c4c(c5c(N6CC7CCC(C7)C6)cc(OC[C@@]67CCCN6C[C@H](F)C7)cc5c3C)CCC4)c21]

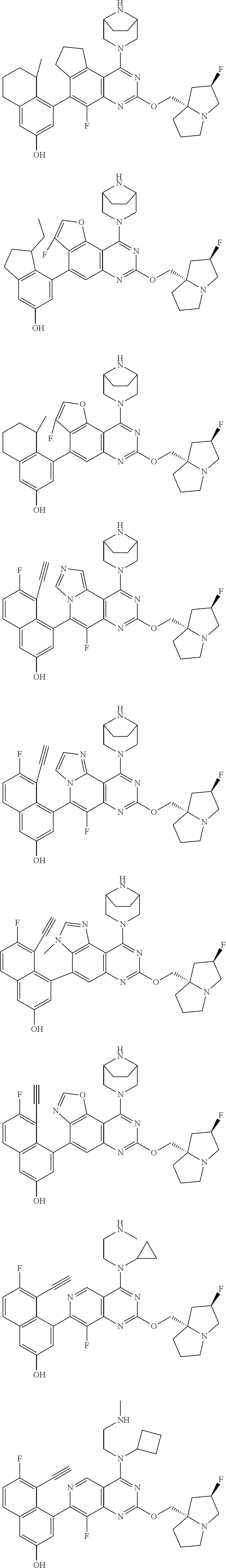

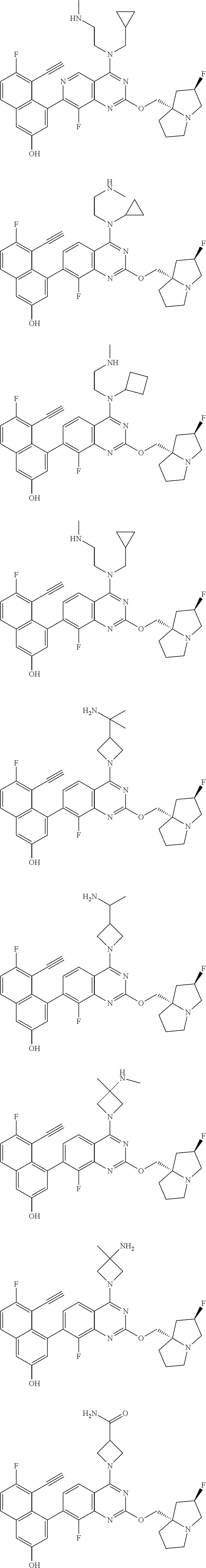

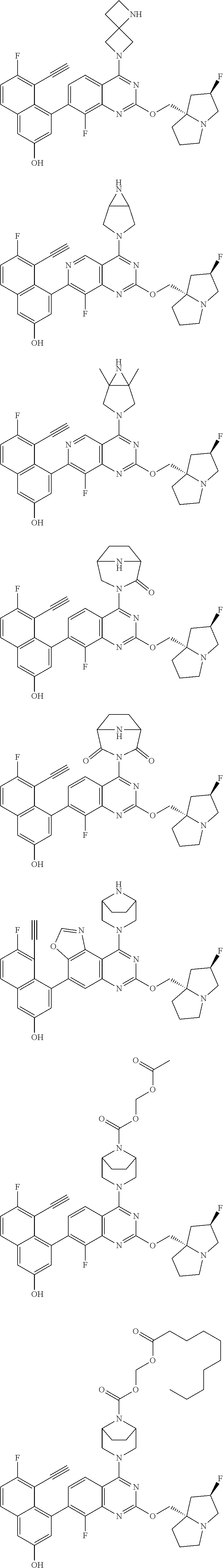

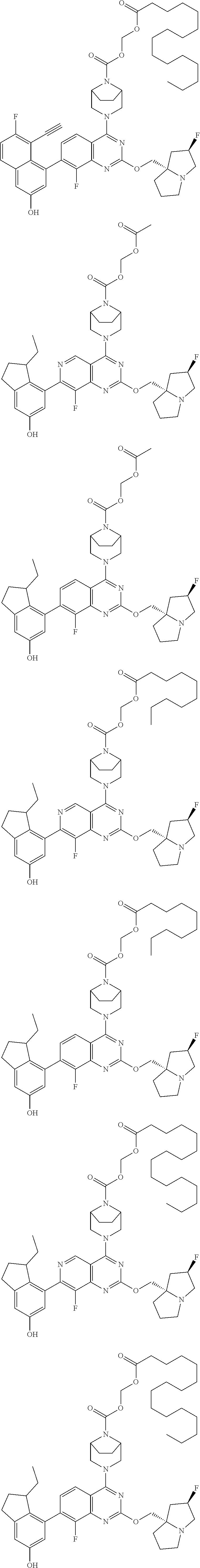

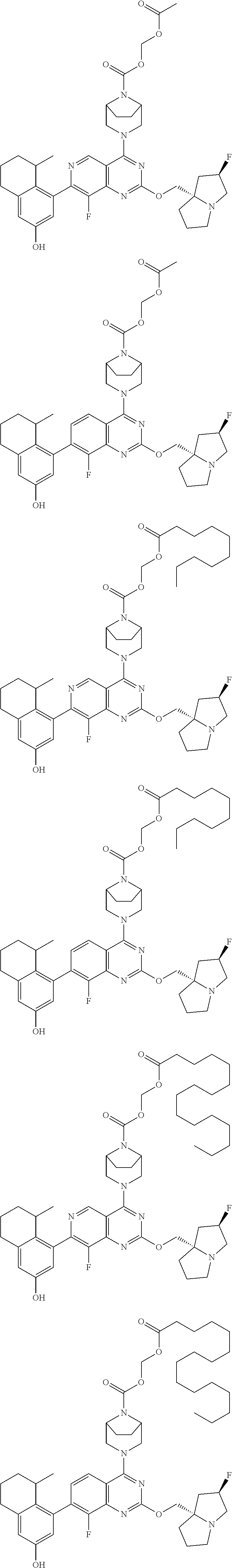

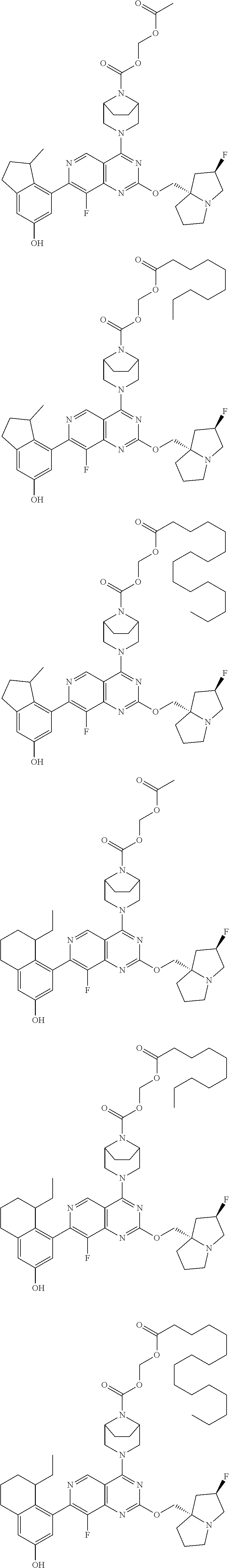

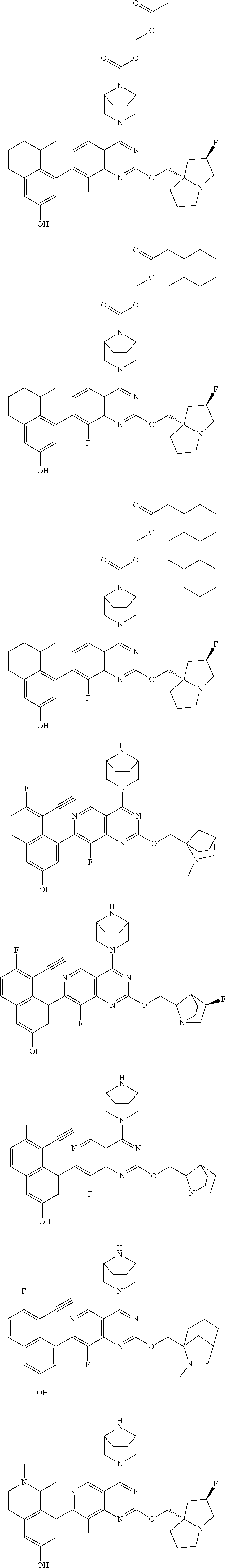

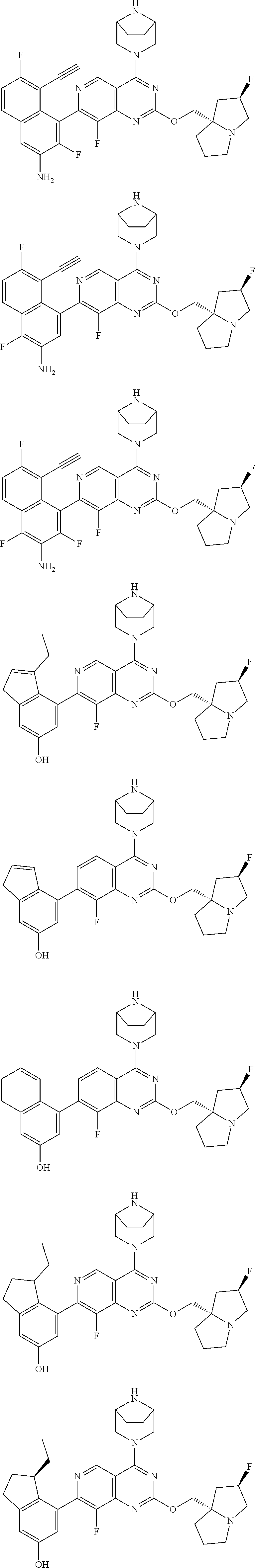

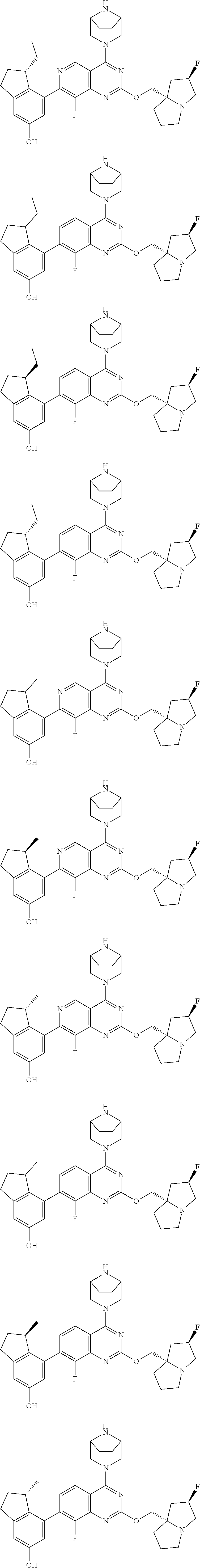

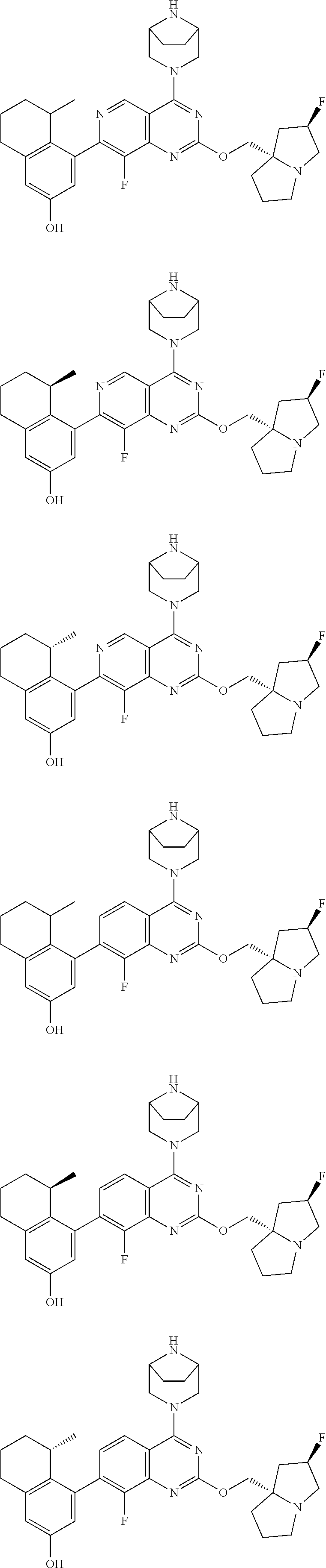

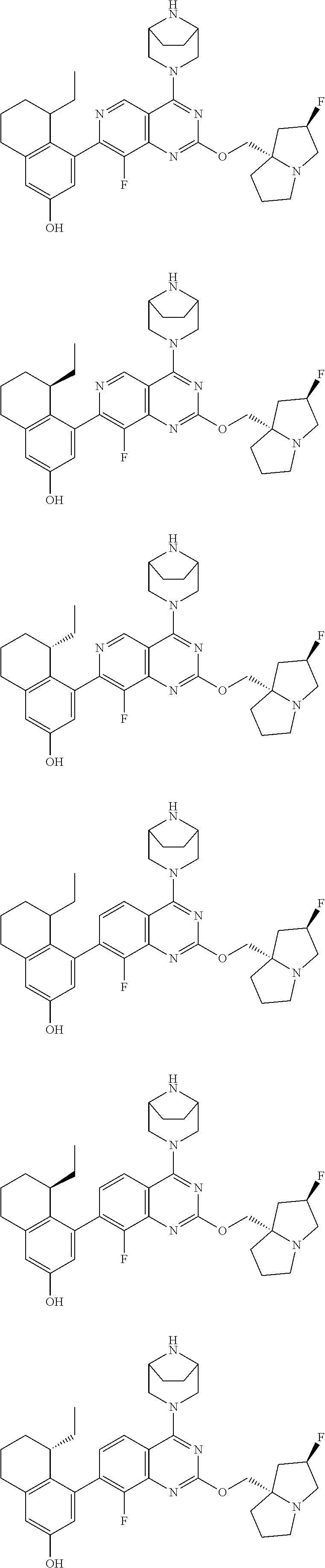

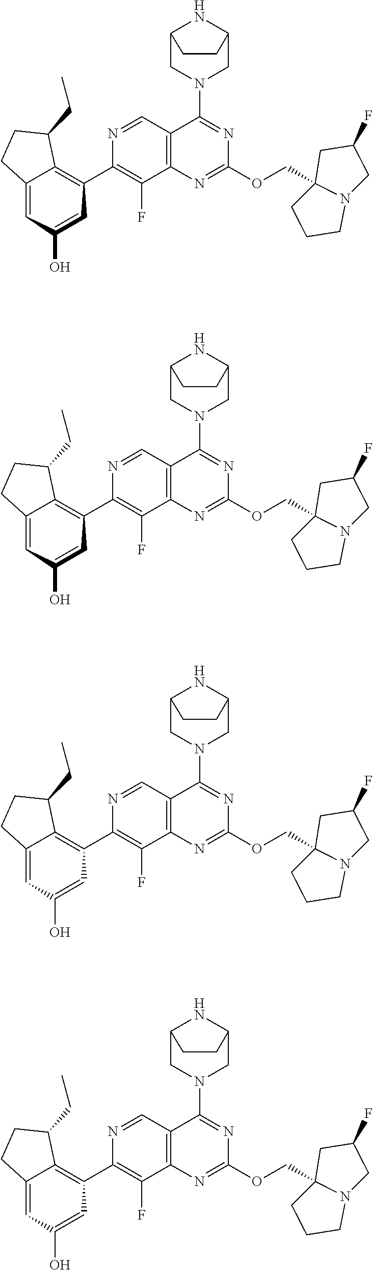

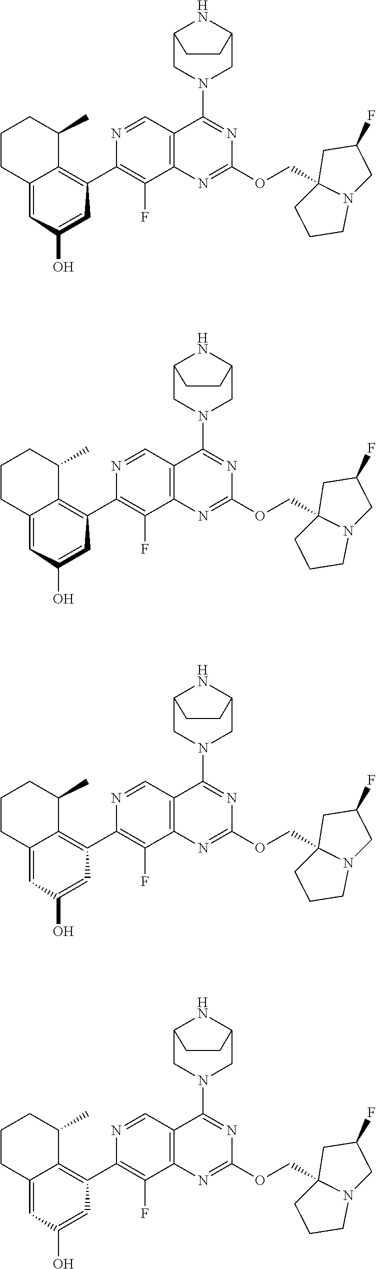

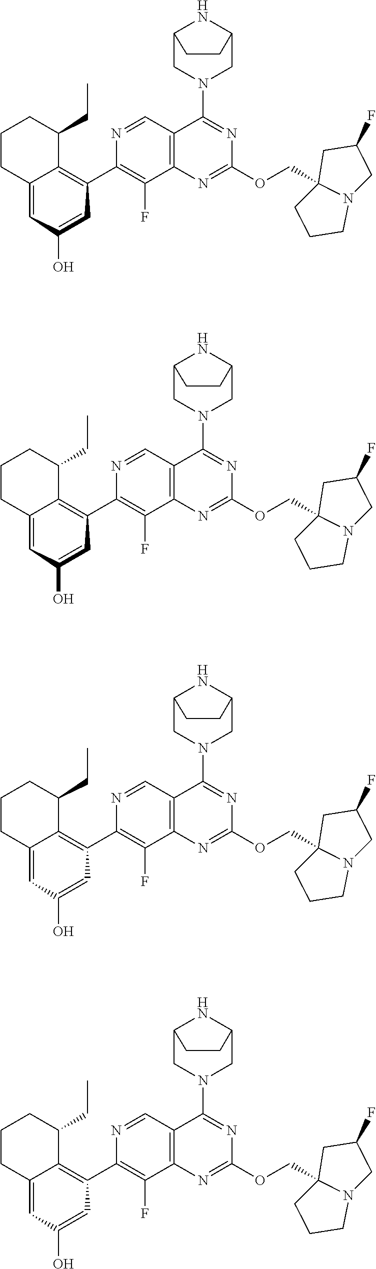

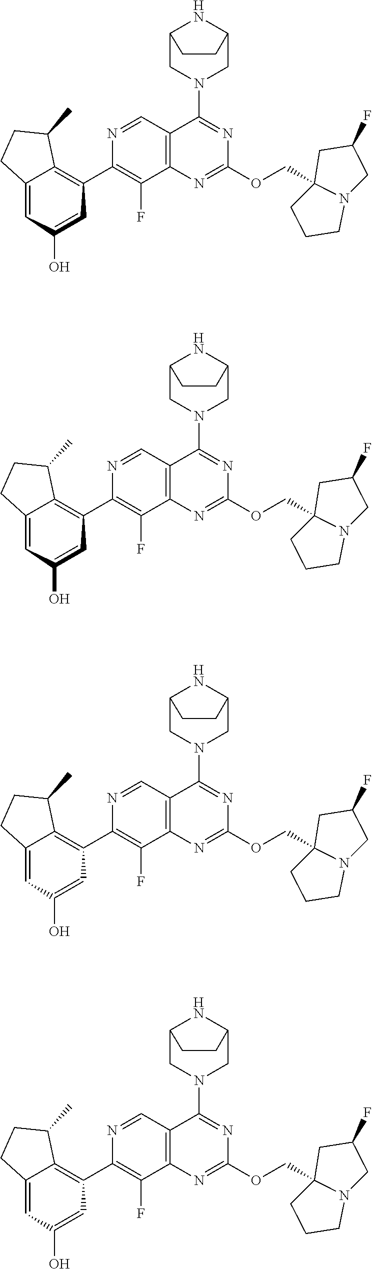

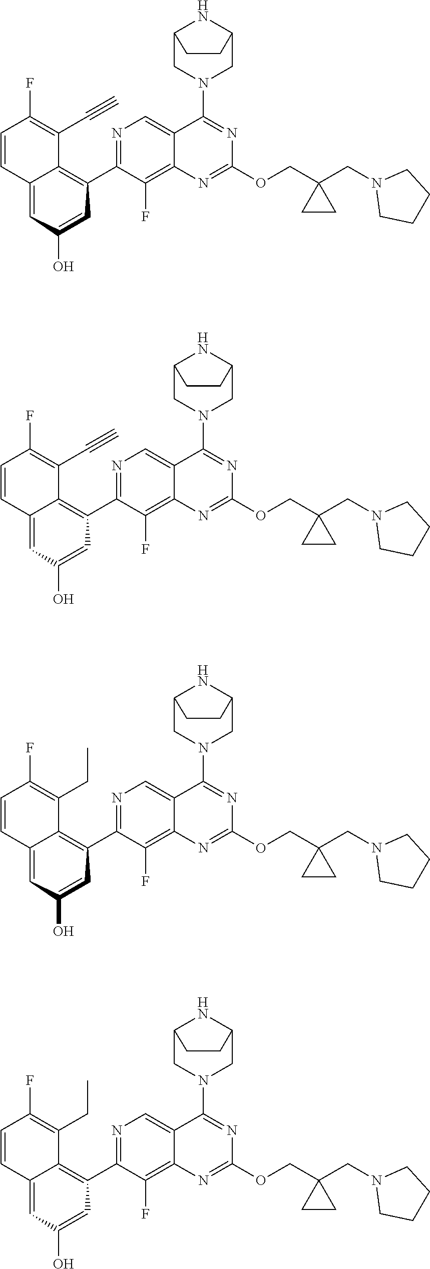

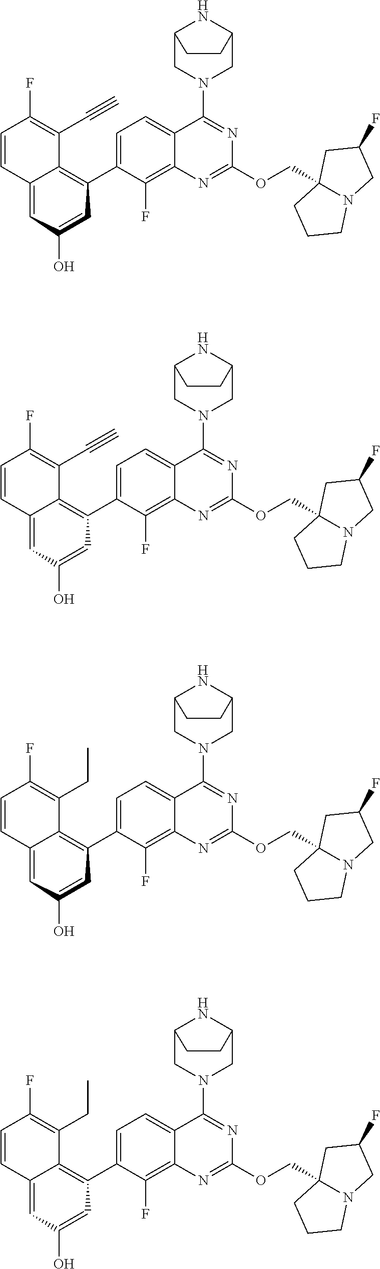

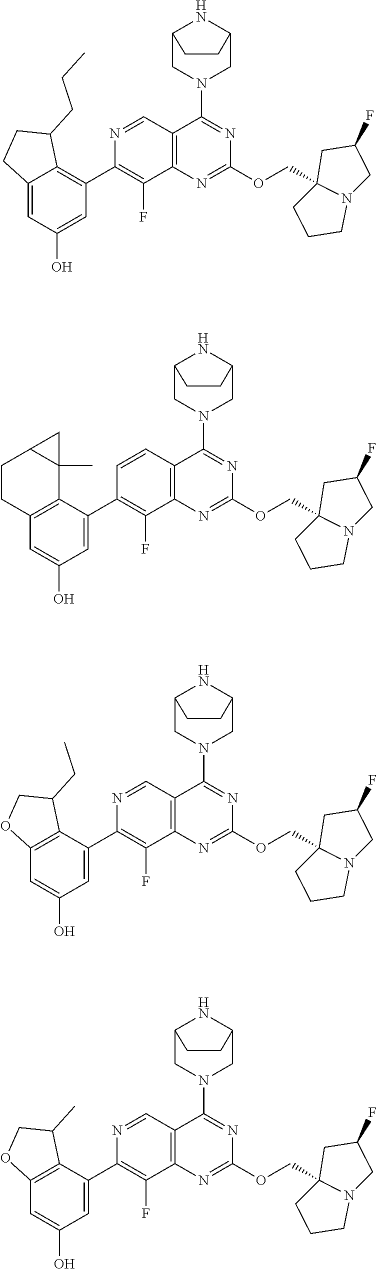 [SMILES: CCCC1CCc2cc(O)cc(-c3ncc4c(N5CC6CCC(C5)N6)nc(OC[C@@]56CCCN5C[C@H](F)C6)nc4c3F)c21 ; CC12CC1CCc1cc(O)cc(-c3ccc4c(N5CC6CCC(C5)N6)nc(OC[C@@]56CCCN5C[C@H](F)C6)nc4c3F)c12 ; CCC1COc2cc(O)cc(-c3ncc4c(N5CC6CCC(C5)N6)nc(OC[C@@]56CCCN5C[C@H](F)C6)nc4c3F)c21 ; CC1COc2cc(O)cc(-c3ncc4c(N5CC6CCC(C5)N6)nc(OC[C@@]56CCCN5C[C@H](F)C6)nc4c3F)c21]

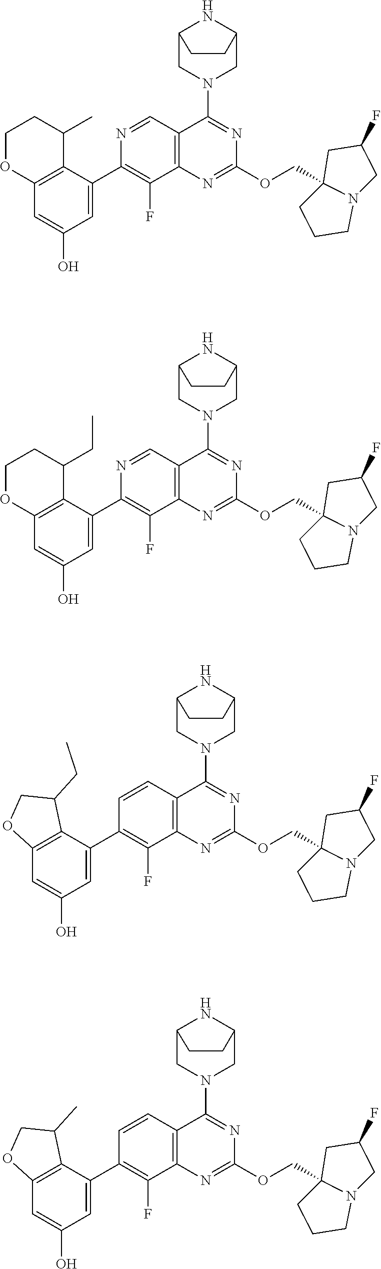 [SMILES: CC1CCOc2cc(O)cc(-c3ncc4c(N5CC6CCC(C5)N6)nc(OC[C@@]56CCCN5C[C@H](F)C6)nc4c3F)c21 ; CCC1CCOc2cc(O)cc(-c3ncc4c(N5CC6CCC(C5)N6)nc(OC[C@@]56CCCN5C[C@H](F)C6)nc4c3F)c21 ; CCC1COc2cc(O)cc(-c3ccc4c(N5CC6CCC(C5)N6)nc(OC[C@@]56CCCN5C[C@H](F)C6)nc4c3F)c21 ; CC1COc2cc(O)cc(-c3ccc4c(N5CC6CCC(C5)N6)nc(OC[C@@]56CCCN5C[C@H](F)C6)nc4c3F)c21]

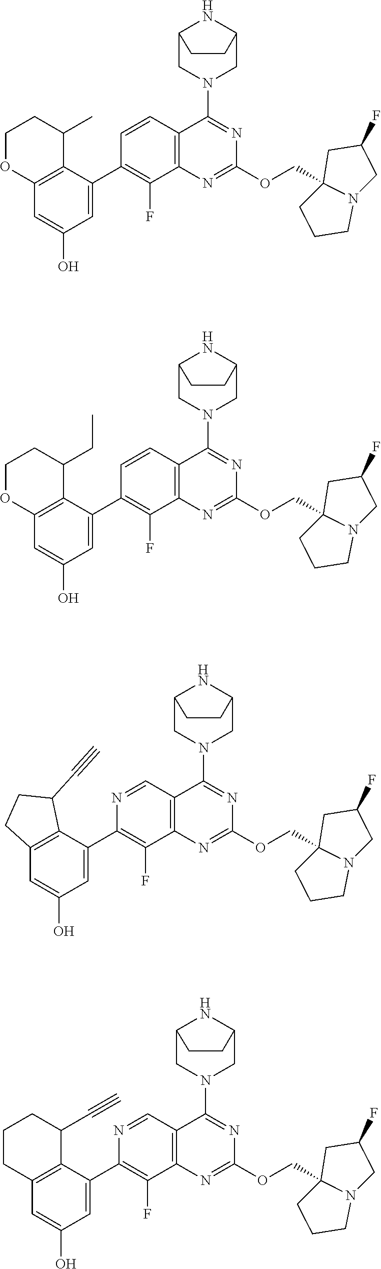 [SMILES: CC1CCOc2cc(O)cc(-c3ccc4c(N5CC6CCC(C5)N6)nc(OC[C@@]56CCCN5C[C@H](F)C6)nc4c3F)c21 ; CCC1CCOc2cc(O)cc(-c3ccc4c(N5CC6CCC(C5)N6)nc(OC[C@@]56CCCN5C[C@H](F)C6)nc4c3F)c21 ; C#CC1CCc2cc(O)cc(-c3ncc4c(N5CC6CCC(C5)N6)nc(OC[C@@]56CCCN5C[C@H](F)C6)nc4c3F)c21 ; C#CC1CCCc2cc(O)cc(-c3ncc4c(N5CC6CCC(C5)N6)nc(OC[C@@]56CCCN5C[C@H](F)C6)nc4c3F)c21]

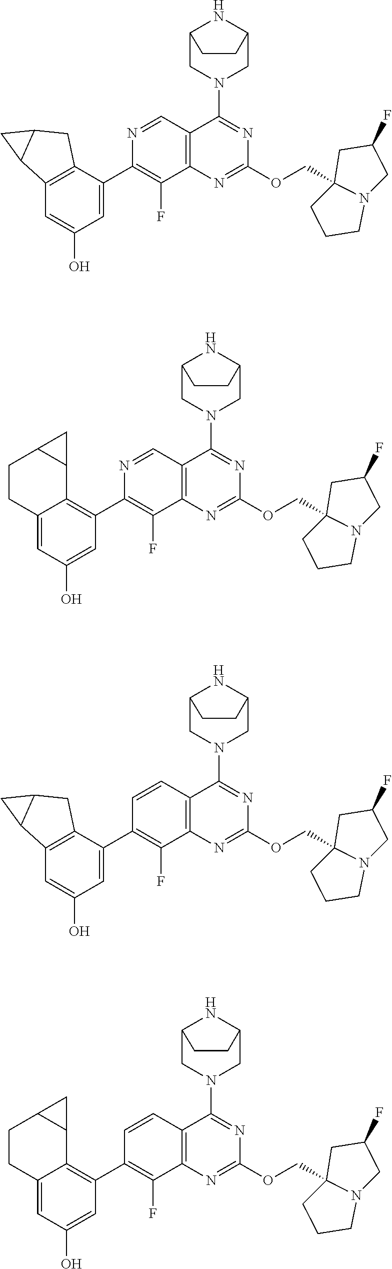 [SMILES: Oc1cc(-c2ncc3c(N4CC5CCC(C4)N5)nc(OC[C@@]45CCCN4C[C@H](F)C5)nc3c2F)c2c(c1)C1CC1C2 ; Oc1cc2c(c(-c3ncc4c(N5CC6CCC(C5)N6)nc(OC[C@@]56CCCN5C[C@H](F)C6)nc4c3F)c1)C1CC1CC2 ; Oc1cc(-c2ccc3c(N4CC5CCC(C4)N5)nc(OC[C@@]45CCCN4C[C@H](F)C5)nc3c2F)c2c(c1)C1CC1C2 ; Oc1cc2c(c(-c3ccc4c(N5CC6CCC(C5)N6)nc(OC[C@@]56CCCN5C[C@H](F)C6)nc4c3F)c1)C1CC1CC2]

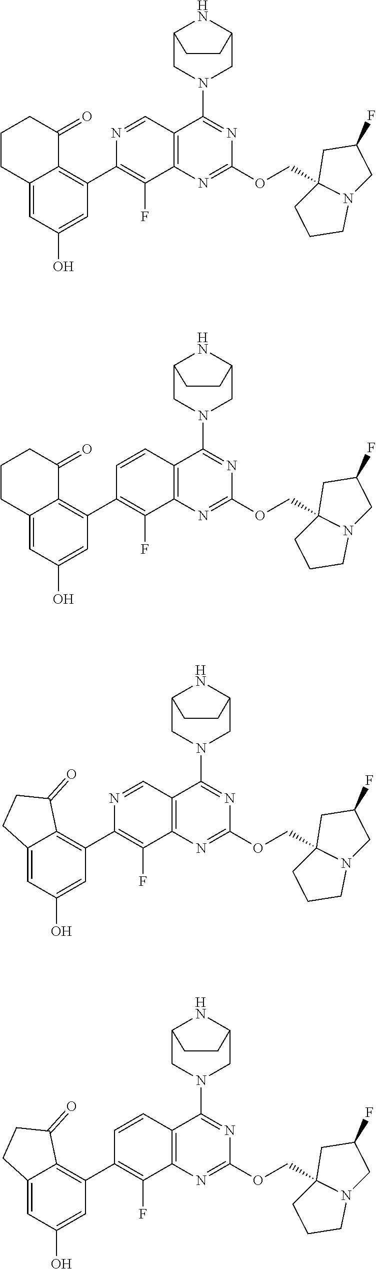 [SMILES: O=C1CCCc2cc(O)cc(-c3ncc4c(N5CC6CCC(C5)N6)nc(OC[C@@]56CCCN5C[C@H](F)C6)nc4c3F)c21 ; O=C1CCCc2cc(O)cc(-c3ccc4c(N5CC6CCC(C5)N6)nc(OC[C@@]56CCCN5C[C@H](F)C6)nc4c3F)c21 ; O=C1CCc2cc(O)cc(-c3ncc4c(N5CC6CCC(C5)N6)nc(OC[C@@]56CCCN5C[C@H](F)C6)nc4c3F)c21 ; O=C1CCc2cc(O)cc(-c3ccc4c(N5CC6CCC(C5)N6)nc(OC[C@@]56CCCN5C[C@H](F)C6)nc4c3F)c21]

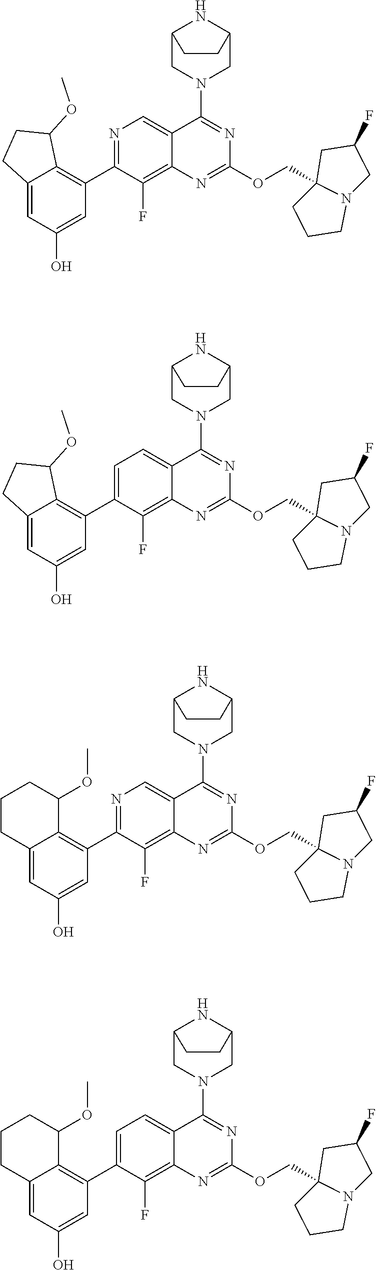 [SMILES: COC1CCc2cc(O)cc(-c3ncc4c(N5CC6CCC(C5)N6)nc(OC[C@@]56CCCN5C[C@H](F)C6)nc4c3F)c21 ; COC1CCc2cc(O)cc(-c3ccc4c(N5CC6CCC(C5)N6)nc(OC[C@@]56CCCN5C[C@H](F)C6)nc4c3F)c21 ; COC1CCCc2cc(O)cc(-c3ncc4c(N5CC6CCC(C5)N6)nc(OC[C@@]56CCCN5C[C@H](F)C6)nc4c3F)c21 ; COC1CCCc2cc(O)cc(-c3ccc4c(N5CC6CCC(C5)N6)nc(OC[C@@]56CCCN5C[C@H](F)C6)nc4c3F)c21]

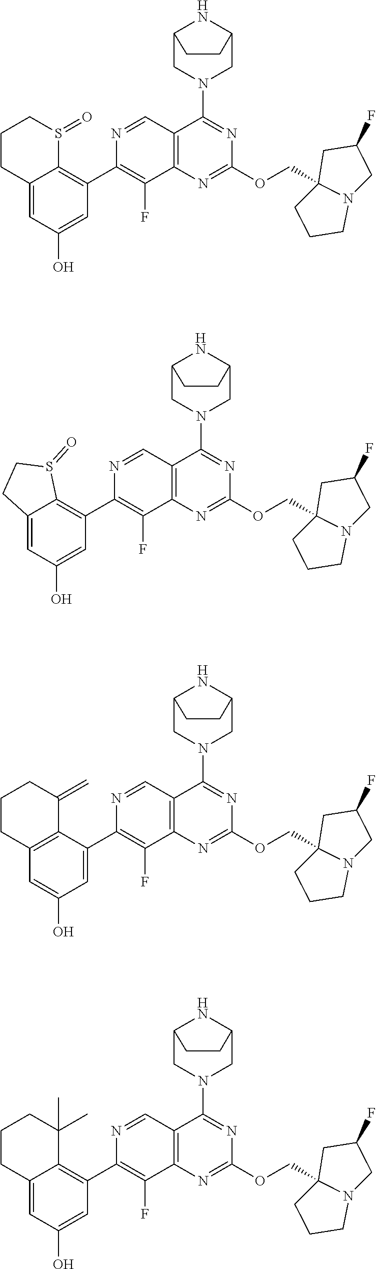 [SMILES: O=S1CCCc2cc(O)cc(-c3ncc4c(N5CC6CCC(C5)N6)nc(OC[C@@]56CCCN5C[C@H](F)C6)nc4c3F)c21 ; O=S1CCc2cc(O)cc(-c3ncc4c(N5CC6CCC(C5)N6)nc(OC[C@@]56CCCN5C[C@H](F)C6)nc4c3F)c21 ; C=C1CCCc2cc(O)cc(-c3ncc4c(N5CC6CCC(C5)N6)nc(OC[C@@]56CCCN5C[C@H](F)C6)nc4c3F)c21 ; CC1(C)CCCc2cc(O)cc(-c3ncc4c(N5CC6CCC(C5)N6)nc(OC[C@@]56CCCN5C[C@H](F)C6)nc4c3F)c21]

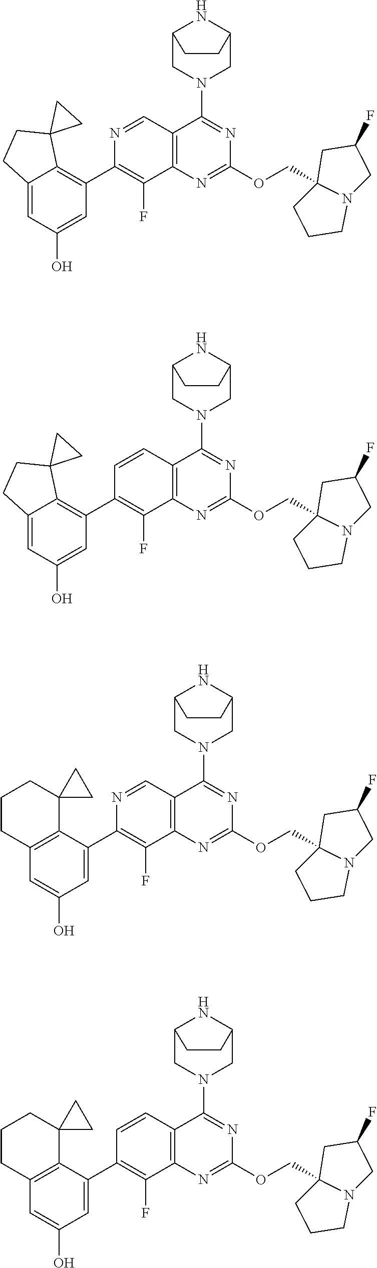 [SMILES: Oc1cc2c(c(-c3ncc4c(N5CC6CCC(C5)N6)nc(OC[C@@]56CCCN5C[C@H](F)C6)nc4c3F)c1)C1(CC2)CC1 ; Oc1cc2c(c(-c3ccc4c(N5CC6CCC(C5)N6)nc(OC[C@@]56CCCN5C[C@H](F)C6)nc4c3F)c1)C1(CC2)CC1 ; Oc1cc2c(c(-c3ncc4c(N5CC6CCC(C5)N6)nc(OC[C@@]56CCCN5C[C@H](F)C6)nc4c3F)c1)C1(CCC2)CC1 ; Oc1cc2c(c(-c3ccc4c(N5CC6CCC(C5)N6)nc(OC[C@@]56CCCN5C[C@H](F)C6)nc4c3F)c1)C1(CCC2)CC1]

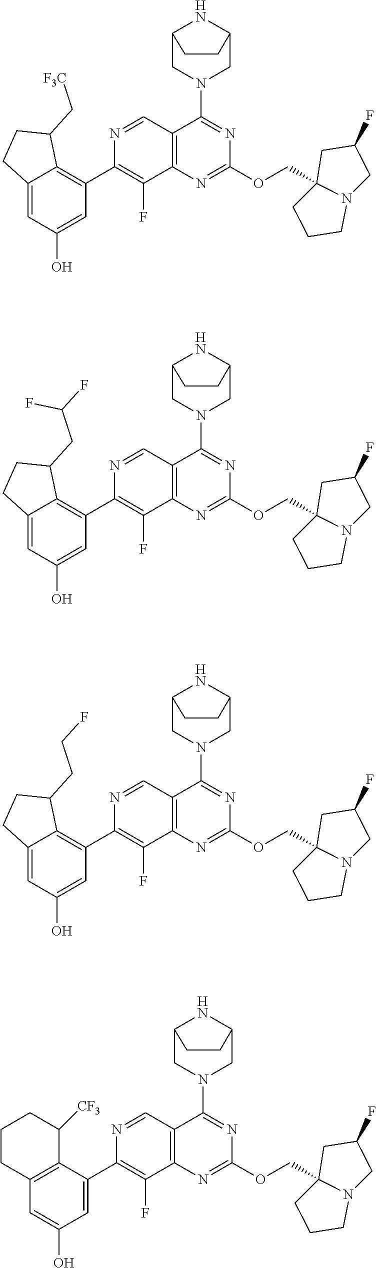 [SMILES: Oc1cc2c(c(-c3ncc4c(N5CC6CCC(C5)N6)nc(OC[C@@]56CCCN5C[C@H](F)C6)nc4c3F)c1)C(CC(F)(F)F)CC2 ; Oc1cc2c(c(-c3ncc4c(N5CC6CCC(C5)N6)nc(OC[C@@]56CCCN5C[C@H](F)C6)nc4c3F)c1)C(CC(F)F)CC2 ; Oc1cc2c(c(-c3ncc4c(N5CC6CCC(C5)N6)nc(OC[C@@]56CCCN5C[C@H](F)C6)nc4c3F)c1)C(CCF)CC2 ; Oc1cc2c(c(-c3ncc4c(N5CC6CCC(C5)N6)nc(OC[C@@]56CCCN5C[C@H](F)C6)nc4c3F)c1)C(C(F)(F)F)CCC2]

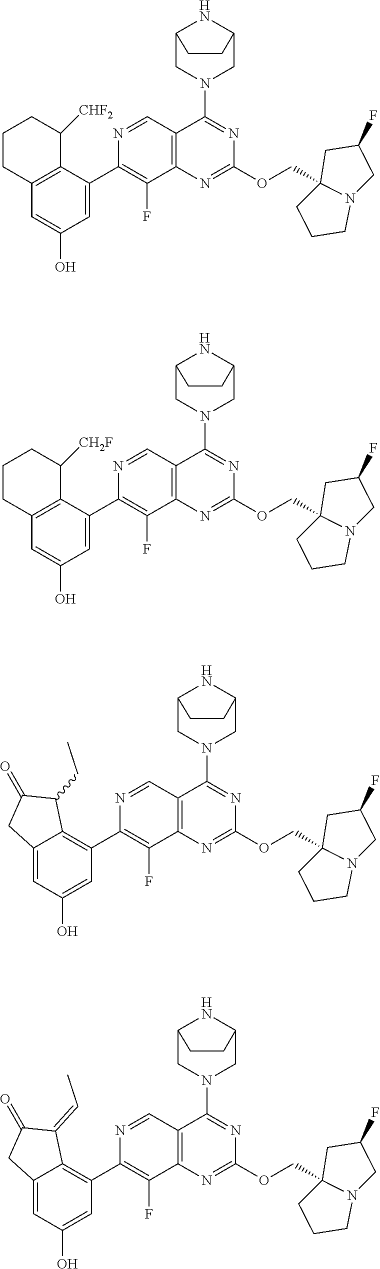

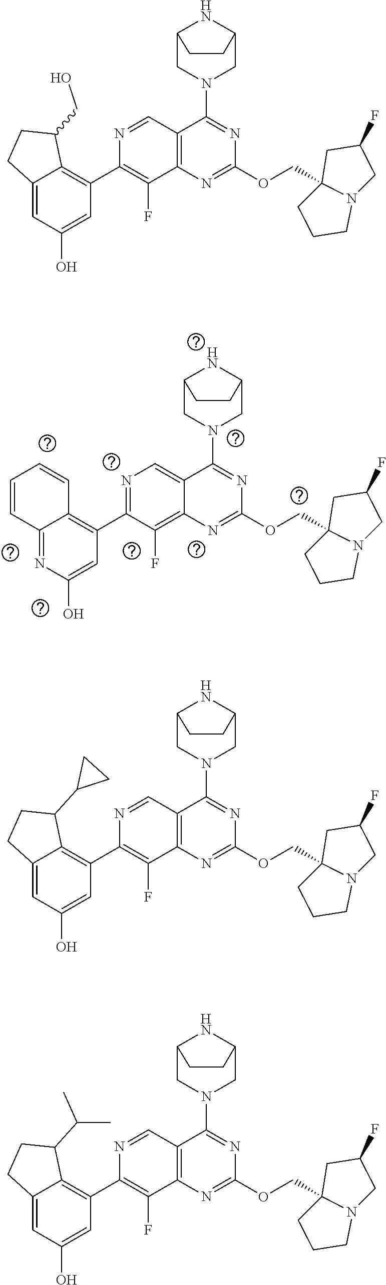 [SMILES: OCC1CCc2cc(O)cc(-c3ncc4c(N5CC6CCC(C5)N6)nc(OC[C@@]56CCCN5C[C@H](F)C6)nc4c3F)c21 ; Oc1cc(-c2ncc3c(N4CC5CCC(C4)N5)nc(OC[C@@]45CCCN4C[C@H](F)C5)nc3c2F)c2ccccc2n1 ; Oc1cc2c(c(-c3ncc4c(N5CC6CCC(C5)N6)nc(OC[C@@]56CCCN5C[C@H](F)C6)nc4c3F)c1)C(C1CC1)CC2 ; CC(C)C1CCc2cc(O)cc(-c3ncc4c(N5CC6CCC(C5)N6)nc(OC[C@@]56CCCN5C[C@H](F)C6)nc4c3F)c21]

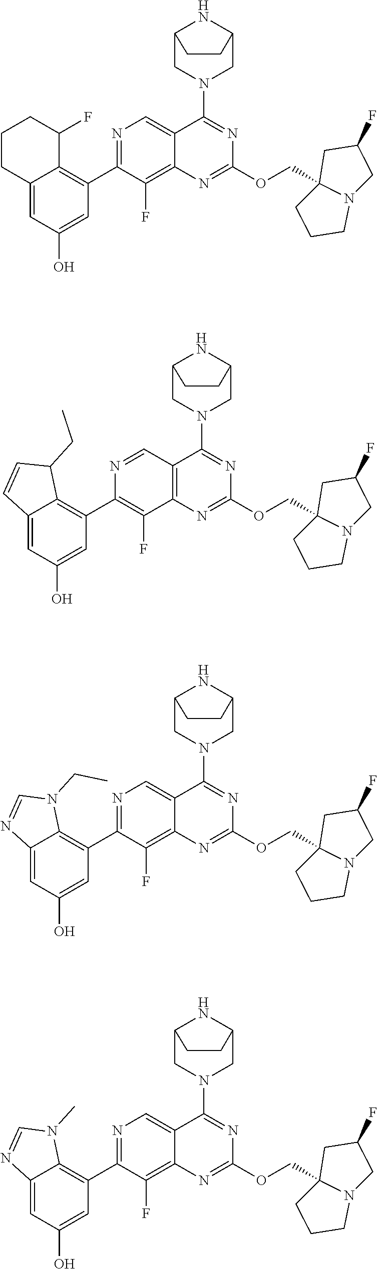 [SMILES: Oc1cc2c(c(-c3ncc4c(N5CC6CCC(C5)N6)nc(OC[C@@]56CCCN5C[C@H](F)C6)nc4c3F)c1)C(F)CCC2 ; CCC1C=Cc2cc(O)cc(-c3ncc4c(N5CC6CCC(C5)N6)nc(OC[C@@]56CCCN5C[C@H](F)C6)nc4c3F)c21 ; CCn1cnc2cc(O)cc(-c3ncc4c(N5CC6CCC(C5)N6)nc(OC[C@@]56CCCN5C[C@H](F)C6)nc4c3F)c21 ; Cn1cnc2cc(O)cc(-c3ncc4c(N5CC6CCC(C5)N6)nc(OC[C@@]56CCCN5C[C@H](F)C6)nc4c3F)c21]

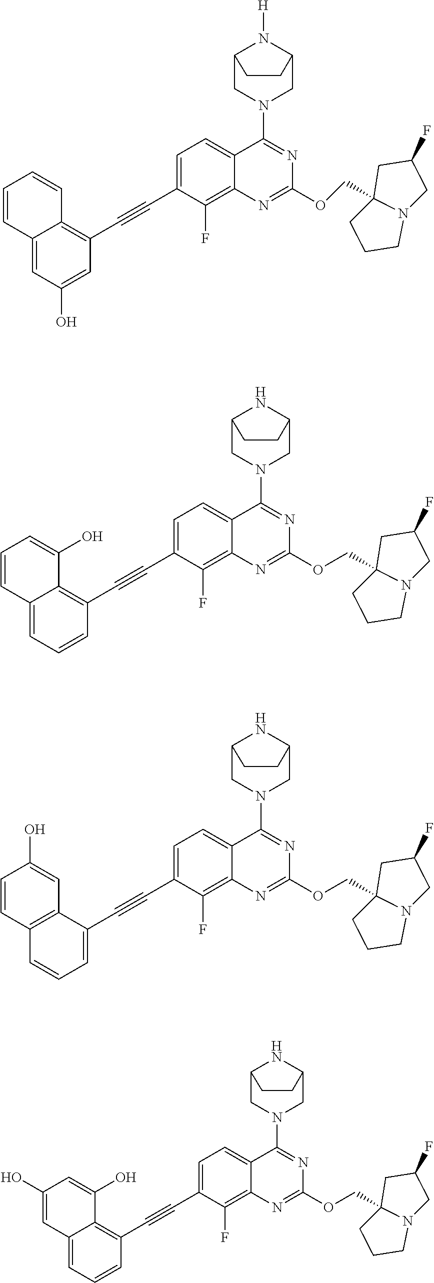 [SMILES: Oc1cc(C#Cc2ccc3c(N4CC5CCC(C4)N5)nc(OC[C@@]45CCCN4C[C@H](F)C5)nc3c2F)c2ccccc2c1 ; Oc1cccc2cccc(C#Cc3ccc4c(N5CC6CCC(C5)N6)nc(OC[C@@]56CCCN5C[C@H](F)C6)nc4c3F)c12 ; Oc1ccc2cccc(C#Cc3ccc4c(N5CC6CCC(C5)N6)nc(OC[C@@]56CCCN5C[C@H](F)C6)nc4c3F)c2c1 ; Oc1cc(O)c2c(C#Cc3ccc4c(N5CC6CCC(C5)N6)nc(OC[C@@]56CCCN5C[C@H](F)C6)nc4c3F)cccc2c1]

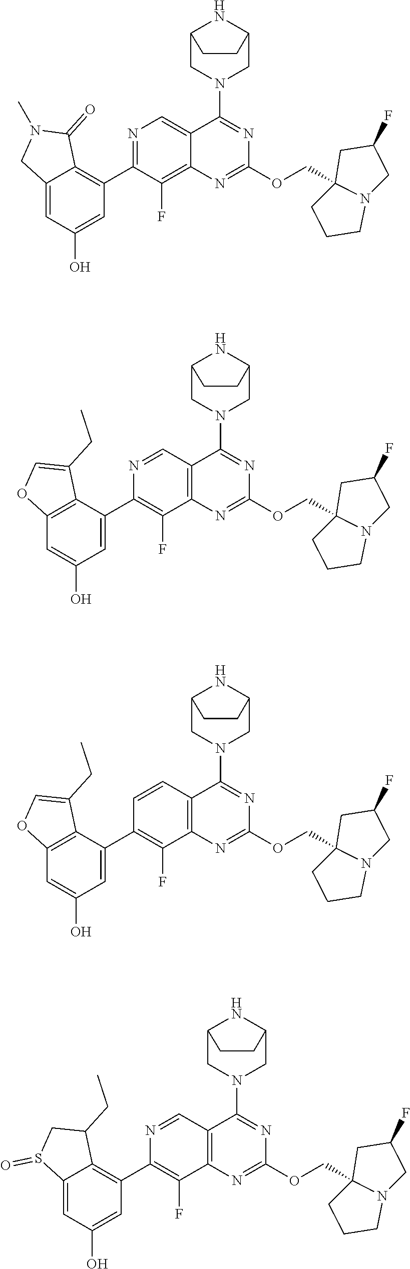 [SMILES: CN1Cc2cc(O)cc(-c3ncc4c(N5CC6CCC(C5)N6)nc(OC[C@@]56CCCN5C[C@H](F)C6)nc4c3F)c2C1=O ; CCc1coc2cc(O)cc(-c3ncc4c(N5CC6CCC(C5)N6)nc(OC[C@@]56CCCN5C[C@H](F)C6)nc4c3F)c12 ; CCc1coc2cc(O)cc(-c3ccc4c(N5CC6CCC(C5)N6)nc(OC[C@@]56CCCN5C[C@H](F)C6)nc4c3F)c12 ; CCC1CS(=O)c2cc(O)cc(-c3ncc4c(N5CC6CCC(C5)N6)nc(OC[C@@]56CCCN5C[C@H](F)C6)nc4c3F)c21]

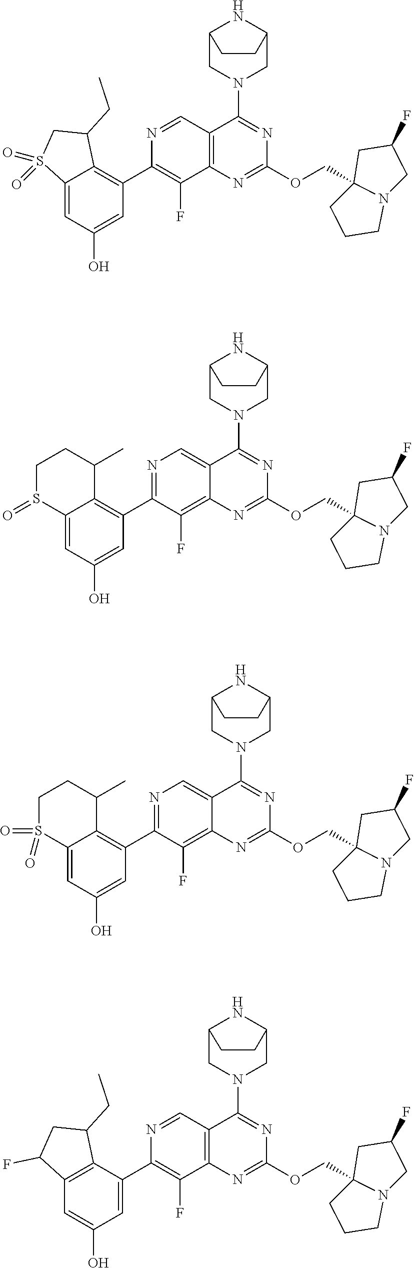 [SMILES: CCC1CS(=O)(=O)c2cc(O)cc(-c3ncc4c(N5CC6CCC(C5)N6)nc(OC[C@@]56CCCN5C[C@H](F)C6)nc4c3F)c21 ; CC1CCS(=O)c2cc(O)cc(-c3ncc4c(N5CC6CCC(C5)N6)nc(OC[C@@]56CCCN5C[C@H](F)C6)nc4c3F)c21 ; CC1CCS(=O)(=O)c2cc(O)cc(-c3ncc4c(N5CC6CCC(C5)N6)nc(OC[C@@]56CCCN5C[C@H](F)C6)nc4c3F)c21 ; CCC1CC(F)c2cc(O)cc(-c3ncc4c(N5CC6CCC(C5)N6)nc(OC[C@@]56CCCN5C[C@H](F)C6)nc4c3F)c21]

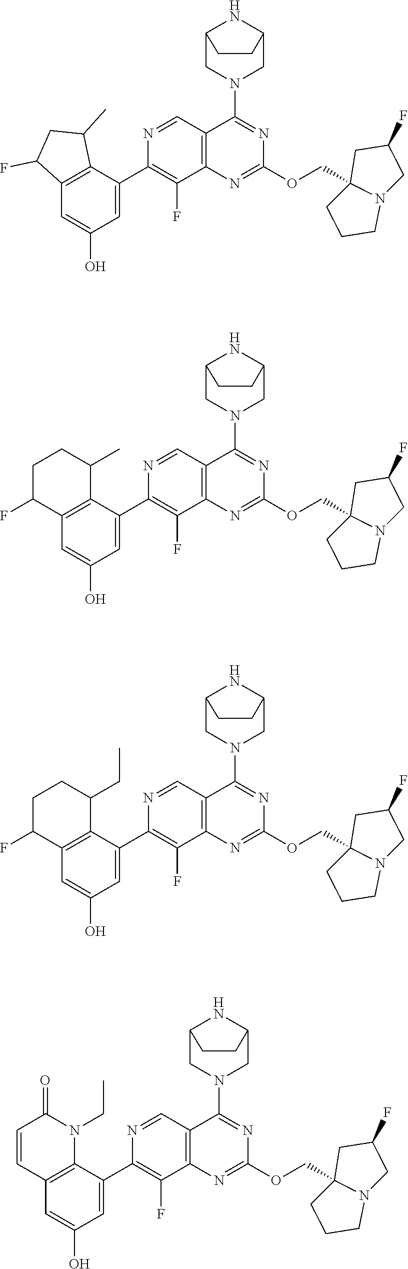 [SMILES: CC1CC(F)c2cc(O)cc(-c3ncc4c(N5CC6CCC(C5)N6)nc(OC[C@@]56CCCN5C[C@H](F)C6)nc4c3F)c21 ; CC1CCC(F)c2cc(O)cc(-c3ncc4c(N5CC6CCC(C5)N6)nc(OC[C@@]56CCCN5C[C@H](F)C6)nc4c3F)c21 ; CCC1CCC(F)c2cc(O)cc(-c3ncc4c(N5CC6CCC(C5)N6)nc(OC[C@@]56CCCN5C[C@H](F)C6)nc4c3F)c21 ; CCn1c(=O)ccc2cc(O)cc(-c3ncc4c(N5CC6CCC(C5)N6)nc(OC[C@@]56CCCN5C[C@H](F)C6)nc4c3F)c21]

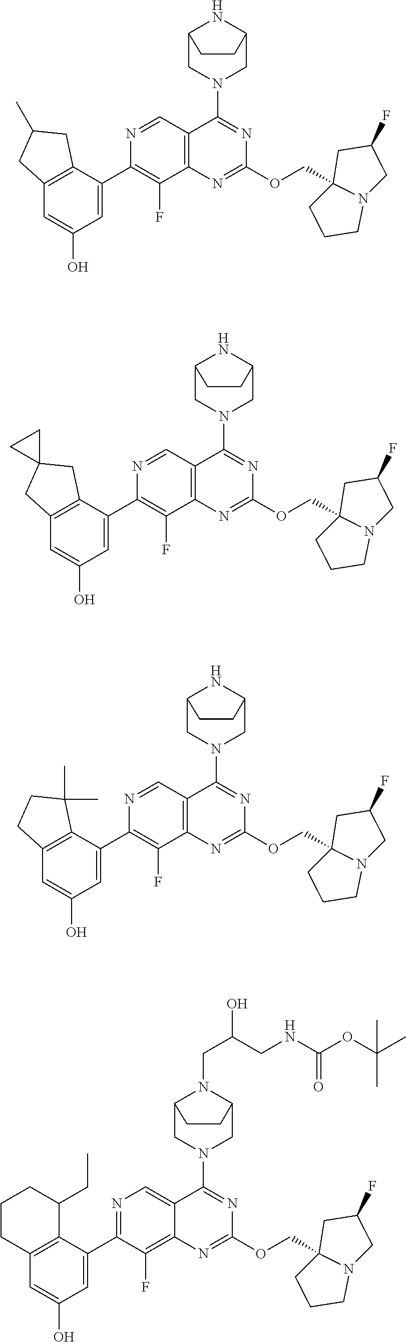

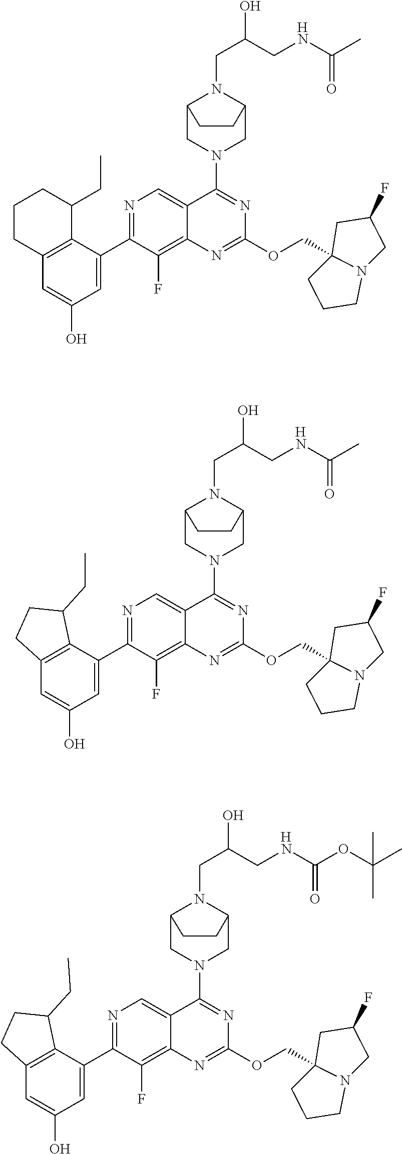

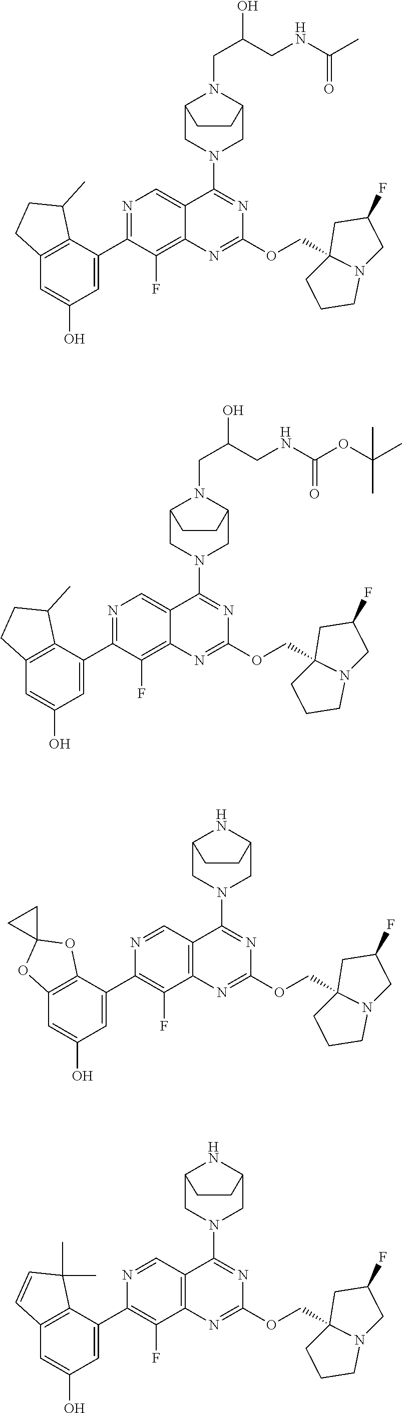

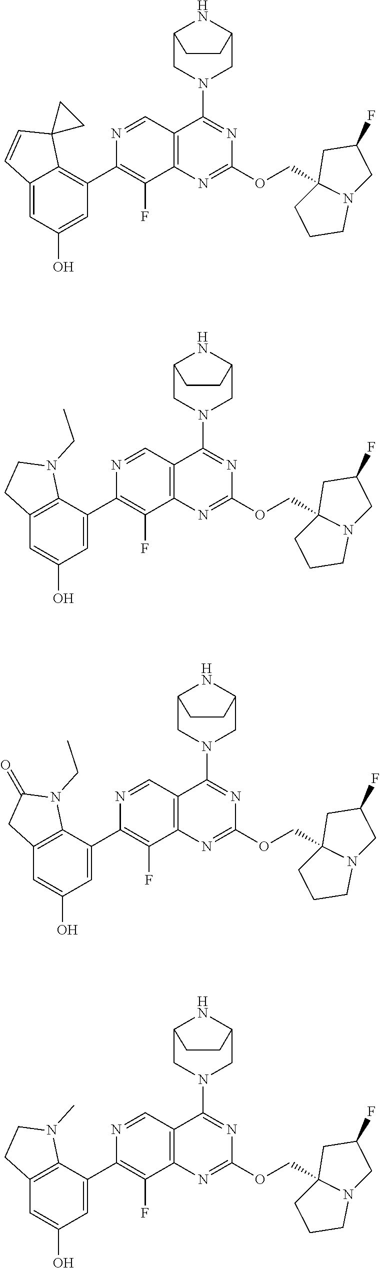 [SMILES: Oc1cc2c(c(-c3ncc4c(N5CC6CCC(C5)N6)nc(OC[C@@]56CCCN5C[C@H](F)C6)nc4c3F)c1)C1(C=C2)CC1 ; CCN1CCc2cc(O)cc(-c3ncc4c(N5CC6CCC(C5)N6)nc(OC[C@@]56CCCN5C[C@H](F)C6)nc4c3F)c21 ; CCN1C(=O)Cc2cc(O)cc(-c3ncc4c(N5CC6CCC(C5)N6)nc(OC[C@@]56CCCN5C[C@H](F)C6)nc4c3F)c21 ; CN1CCc2cc(O)cc(-c3ncc4c(N5CC6CCC(C5)N6)nc(OC[C@@]56CCCN5C[C@H](F)C6)nc4c3F)c21]

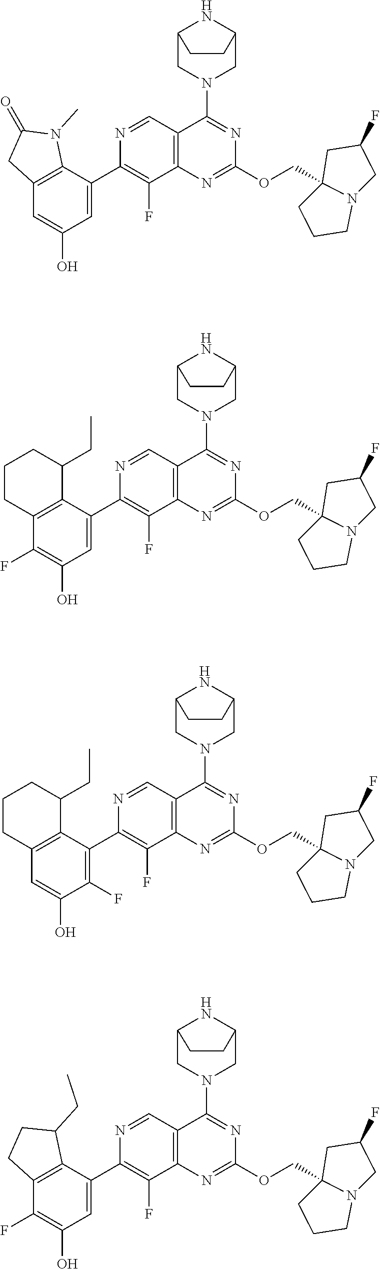 [SMILES: CN1C(=O)Cc2cc(O)cc(-c3ncc4c(N5CC6CCC(C5)N6)nc(OC[C@@]56CCCN5C[C@H](F)C6)nc4c3F)c21 ; CCC1CCCc2c(F)c(O)cc(-c3ncc4c(N5CC6CCC(C5)N6)nc(OC[C@@]56CCCN5C[C@H](F)C6)nc4c3F)c21 ; CCC1CCCc2cc(O)c(F)c(-c3ncc4c(N5CC6CCC(C5)N6)nc(OC[C@@]56CCCN5C[C@H](F)C6)nc4c3F)c21 ; CCC1CCc2c(F)c(O)cc(-c3ncc4c(N5CC6CCC(C5)N6)nc(OC[C@@]56CCCN5C[C@H](F)C6)nc4c3F)c21]

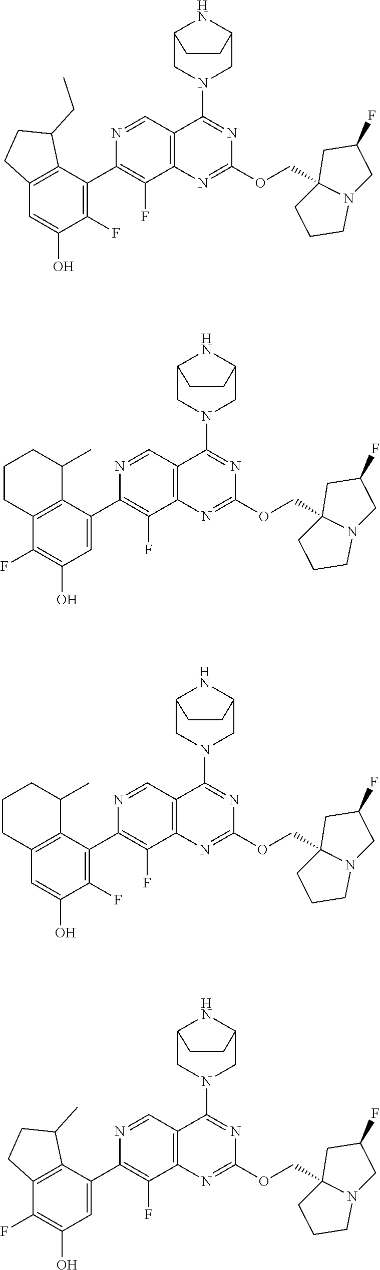 [SMILES: CCC1CCc2cc(O)c(F)c(-c3ncc4c(N5CC6CCC(C5)N6)nc(OC[C@@]56CCCN5C[C@H](F)C6)nc4c3F)c21 ; CC1CCCc2c(F)c(O)cc(-c3ncc4c(N5CC6CCC(C5)N6)nc(OC[C@@]56CCCN5C[C@H](F)C6)nc4c3F)c21 ; CC1CCCc2cc(O)c(F)c(-c3ncc4c(N5CC6CCC(C5)N6)nc(OC[C@@]56CCCN5C[C@H](F)C6)nc4c3F)c21 ; CC1CCc2c(F)c(O)cc(-c3ncc4c(N5CC6CCC(C5)N6)nc(OC[C@@]56CCCN5C[C@H](F)C6)nc4c3F)c21]

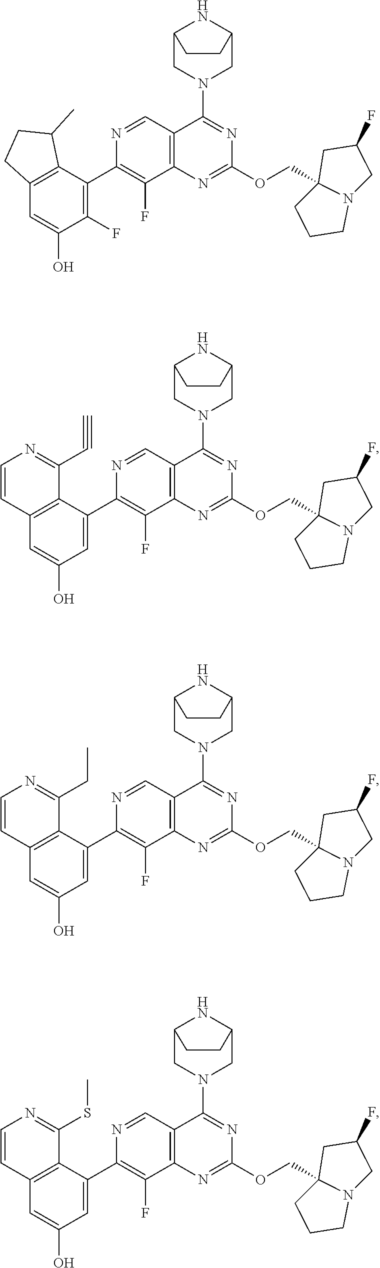 [SMILES: CC1CCc2cc(O)c(F)c(-c3ncc4c(N5CC6CCC(C5)N6)nc(OC[C@@]56CCCN5C[C@H](F)C6)nc4c3F)c21 ; C#Cc1nccc2cc(O)cc(-c3ncc4c(N5CC6CCC(C5)N6)nc(OC[C@@]56CCCN5C[C@H](F)C6)nc4c3F)c12 ; CCc1nccc2cc(O)cc(-c3ncc4c(N5CC6CCC(C5)N6)nc(OC[C@@]56CCCN5C[C@H](F)C6)nc4c3F)c12 ; CSc1nccc2cc(O)cc(-c3ncc4c(N5CC6CCC(C5)N6)nc(OC[C@@]56CCCN5C[C@H](F)C6)nc4c3F)c12]

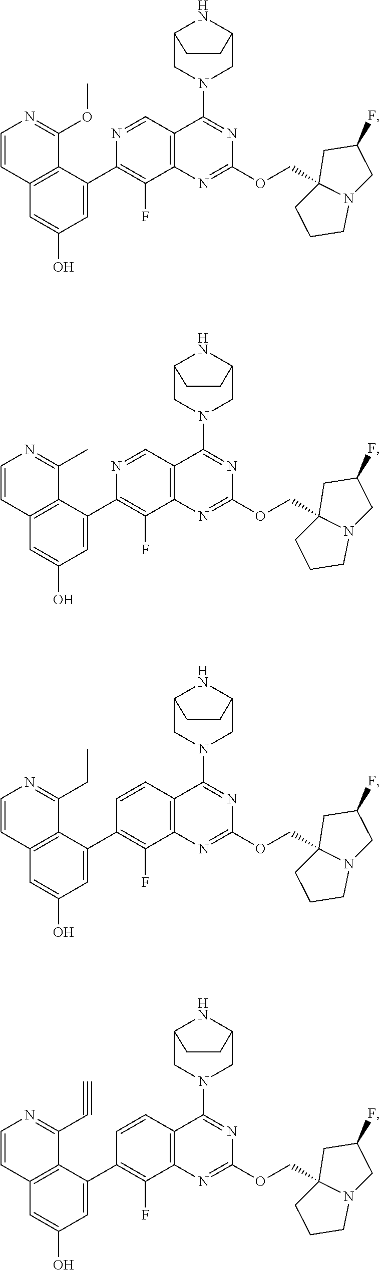 [SMILES: COc1nccc2cc(O)cc(-c3ncc4c(N5CC6CCC(C5)N6)nc(OC[C@@]56CCCN5C[C@H](F)C6)nc4c3F)c12 ; Cc1nccc2cc(O)cc(-c3ncc4c(N5CC6CCC(C5)N6)nc(OC[C@@]56CCCN5C[C@H](F)C6)nc4c3F)c12 ; CCc1nccc2cc(O)cc(-c3ccc4c(N5CC6CCC(C5)N6)nc(OC[C@@]56CCCN5C[C@H](F)C6)nc4c3F)c12 ; C#Cc1nccc2cc(O)cc(-c3ccc4c(N5CC6CCC(C5)N6)nc(OC[C@@]56CCCN5C[C@H](F)C6)nc4c3F)c12]

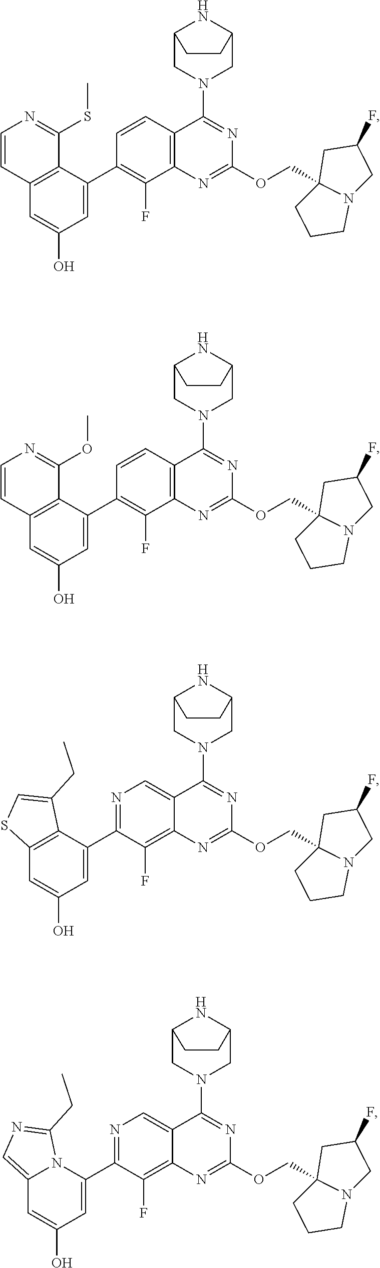 [SMILES: CSc1nccc2cc(O)cc(-c3ccc4c(N5CC6CCC(C5)N6)nc(OC[C@@]56CCCN5C[C@H](F)C6)nc4c3F)c12 ; COc1nccc2cc(O)cc(-c3ccc4c(N5CC6CCC(C5)N6)nc(OC[C@@]56CCCN5C[C@H](F)C6)nc4c3F)c12 ; CCc1csc2cc(O)cc(-c3ncc4c(N5CC6CCC(C5)N6)nc(OC[C@@]56CCCN5C[C@H](F)C6)nc4c3F)c12 ; CCc1ncc2cc(O)cc(-c3ncc4c(N5CC6CCC(C5)N6)nc(OC[C@@]56CCCN5C[C@H](F)C6)nc4c3F)n12]

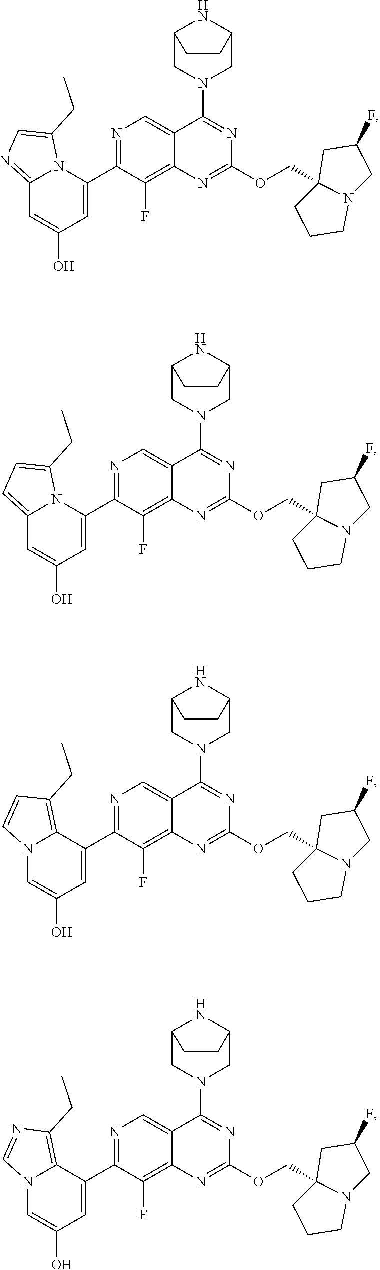 [SMILES: CCc1cnc2cc(O)cc(-c3ncc4c(N5CC6CCC(C5)N6)nc(OC[C@@]56CCCN5C[C@H](F)C6)nc4c3F)n12 ; CCc1ccc2cc(O)cc(-c3ncc4c(N5CC6CCC(C5)N6)nc(OC[C@@]56CCCN5C[C@H](F)C6)nc4c3F)n12 ; CCc1ccn2cc(O)cc(-c3ncc4c(N5CC6CCC(C5)N6)nc(OC[C@@]56CCCN5C[C@H](F)C6)nc4c3F)c12 ; CCc1ncn2cc(O)cc(-c3ncc4c(N5CC6CCC(C5)N6)nc(OC[C@@]56CCCN5C[C@H](F)C6)nc4c3F)c12]

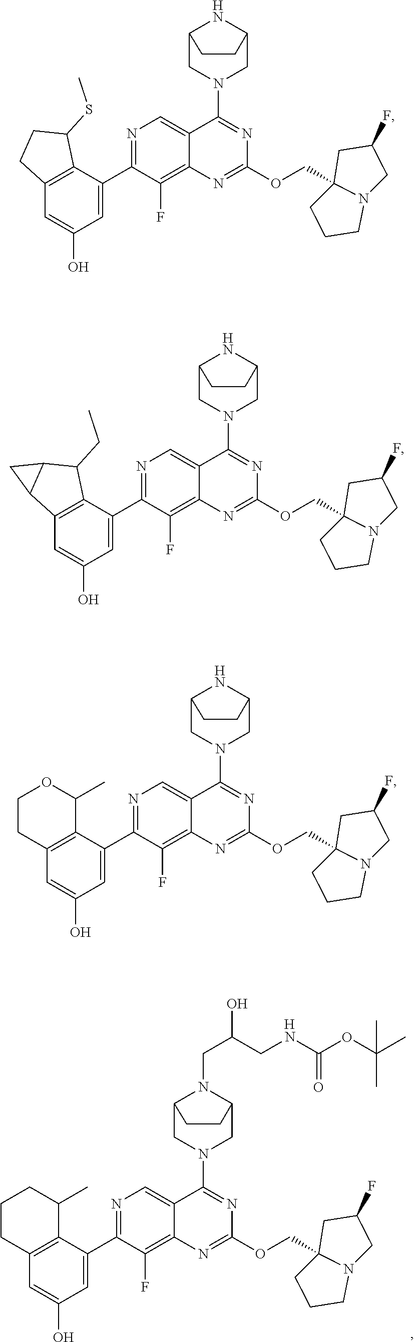

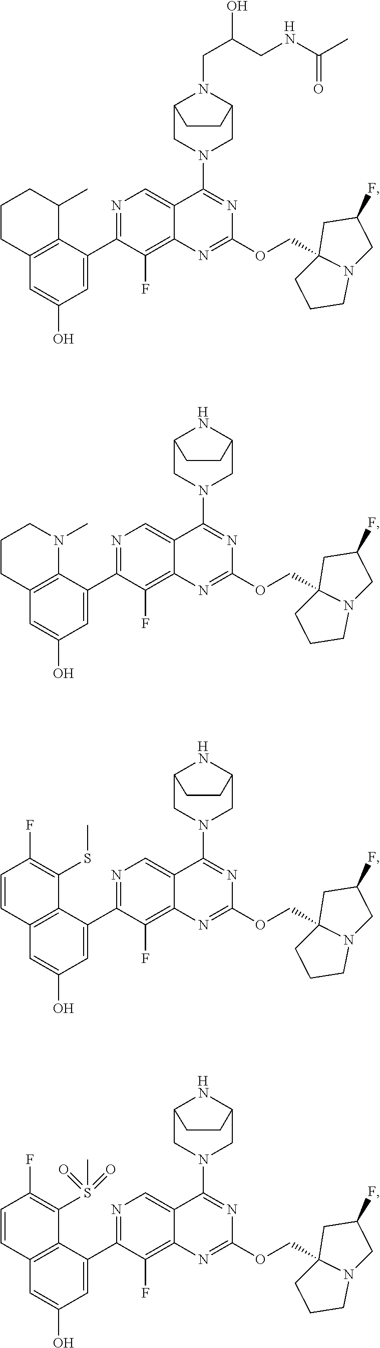

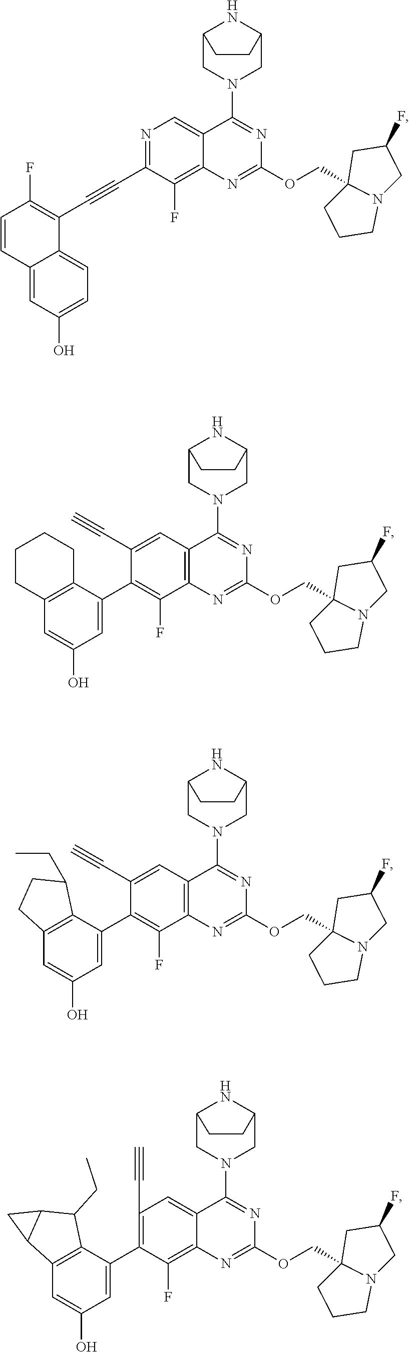

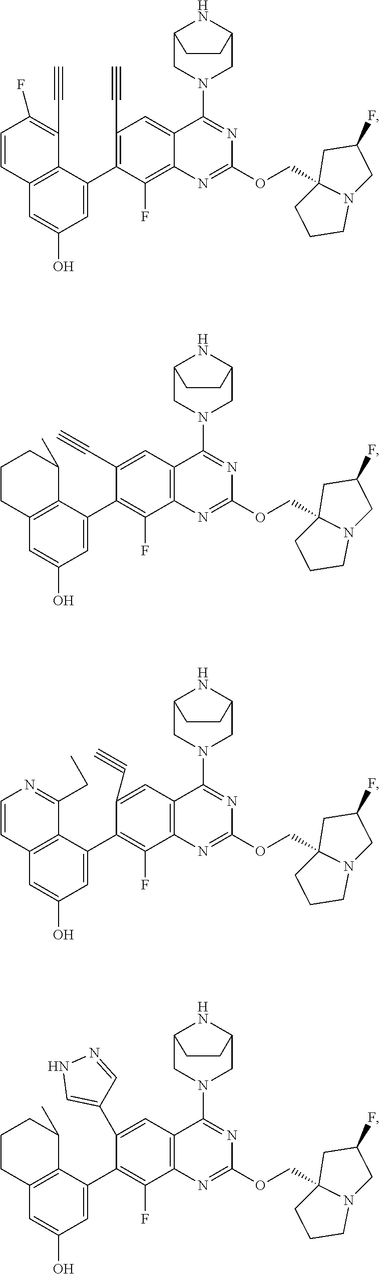

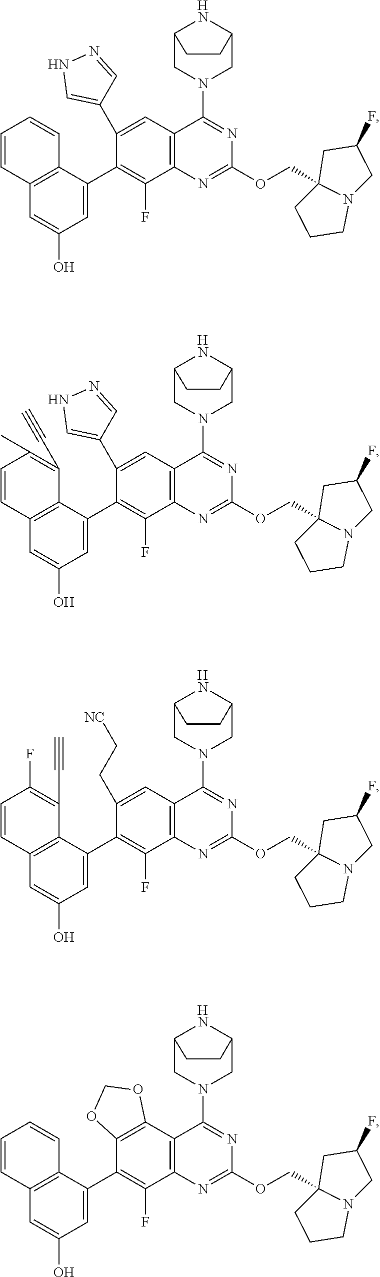

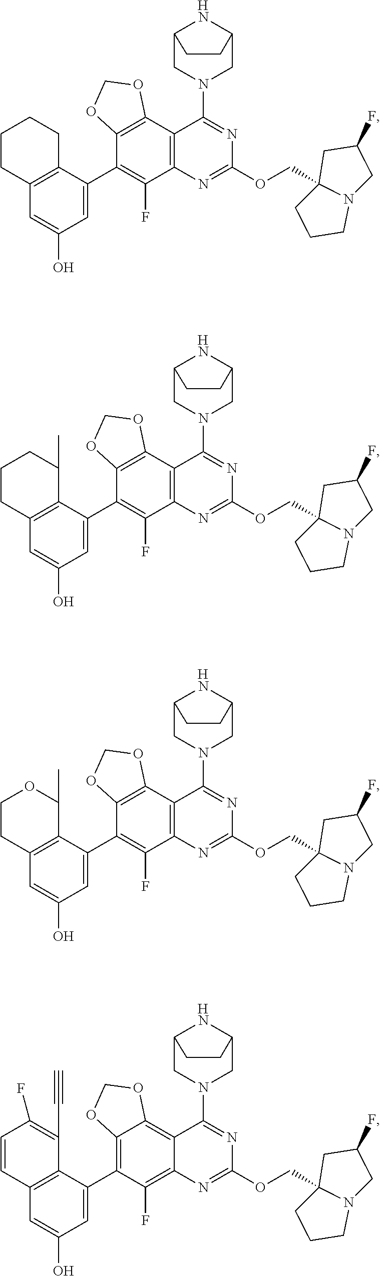

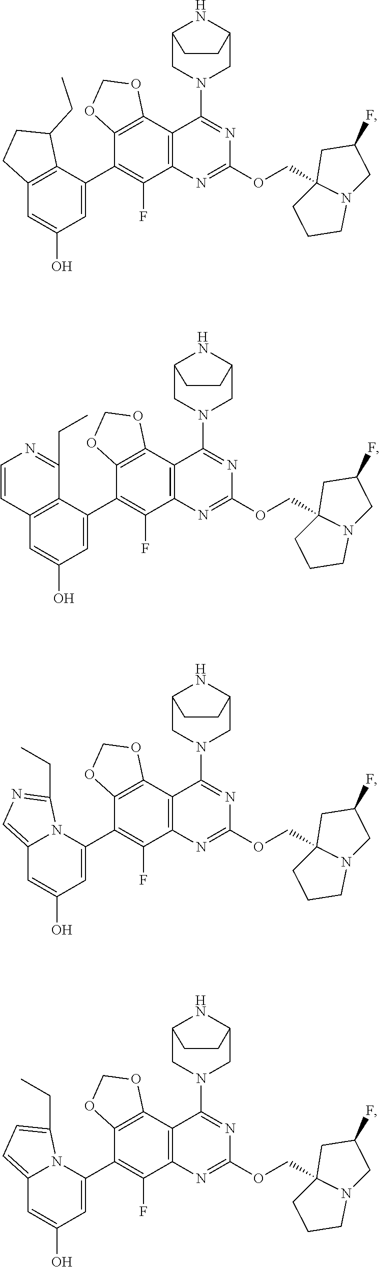

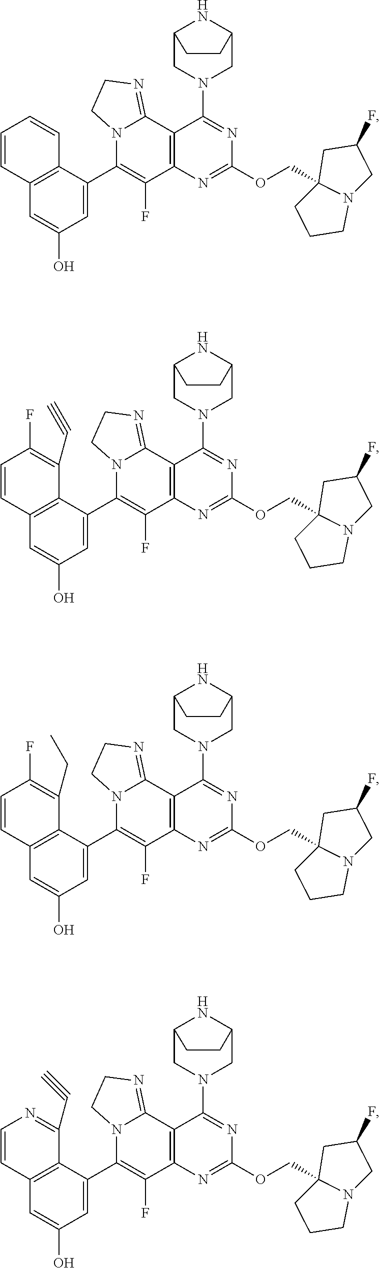 [SMILES: Oc1cc(C2=C(F)c3nc(OC[C@@]45CCCN4C[C@H](F)C5)nc(N4CC5CCC(C4)N5)c3C3=NCCN32)c2ccccc2c1 ; C#Cc1c(F)ccc2cc(O)cc(C3=C(F)c4nc(OC[C@@]56CCCN5C[C@H](F)C6)nc(N5CC6CCC(C5)N6)c4C4=NCCN43)c12 ; CCc1c(F)ccc2cc(O)cc(C3=C(F)c4nc(OC[C@@]56CCCN5C[C@H](F)C6)nc(N5CC6CCC(C5)N6)c4C4=NCCN43)c12 ; C#Cc1nccc2cc(O)cc(C3=C(F)c4nc(OC[C@@]56CCCN5C[C@H](F)C6)nc(N5CC6CCC(C5)N6)c4C4=NCCN43)c12]

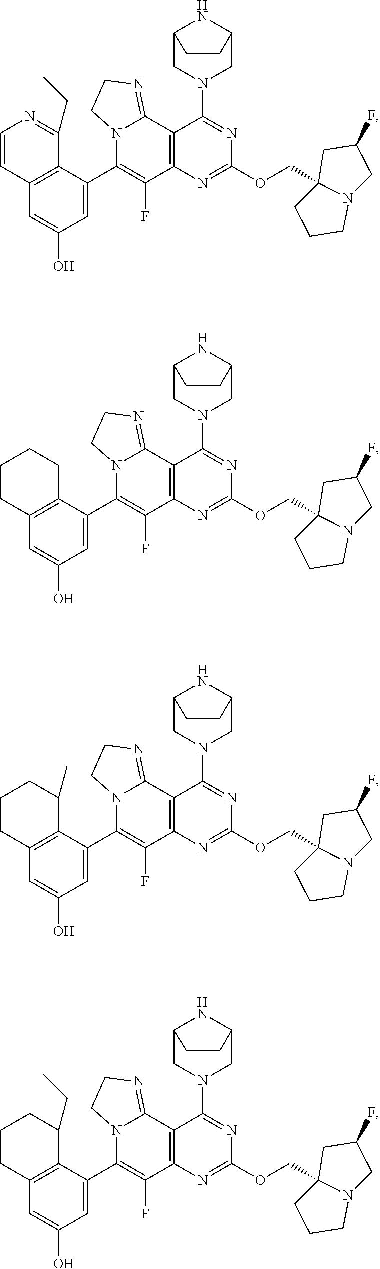 [SMILES: CCc1nccc2cc(O)cc(C3=C(F)c4nc(OC[C@@]56CCCN5C[C@H](F)C6)nc(N5CC6CCC(C5)N6)c4C4=NCCN43)c12 ; Oc1cc2c(c(C3=C(F)c4nc(OC[C@@]56CCCN5C[C@H](F)C6)nc(N5CC6CCC(C5)N6)c4C4=NCCN43)c1)CCCC2 ; CC1CCCc2cc(O)cc(C3=C(F)c4nc(OC[C@@]56CCCN5C[C@H](F)C6)nc(N5CC6CCC(C5)N6)c4C4=NCCN43)c21 ; CCC1CCCc2cc(O)cc(C3=C(F)c4nc(OC[C@@]56CCCN5C[C@H](F)C6)nc(N5CC6CCC(C5)N6)c4C4=NCCN43)c21]

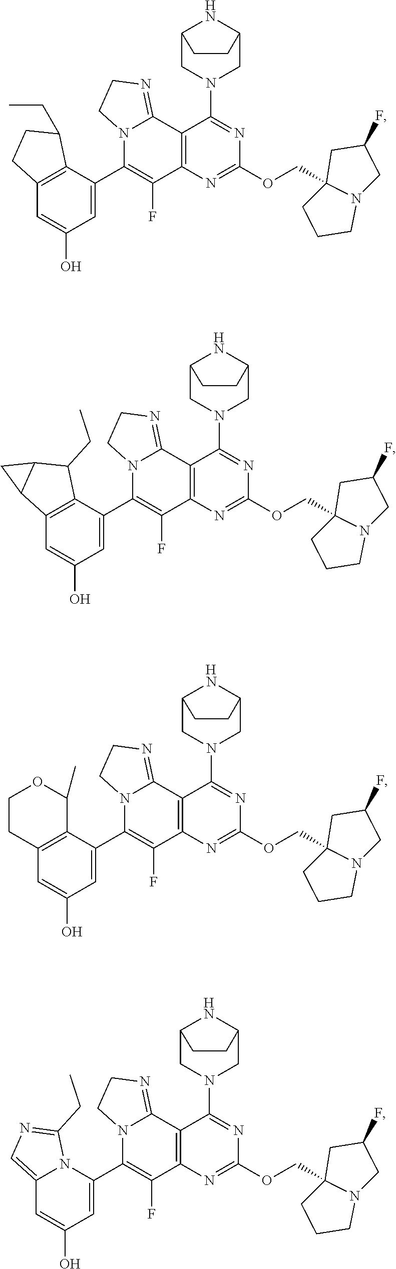

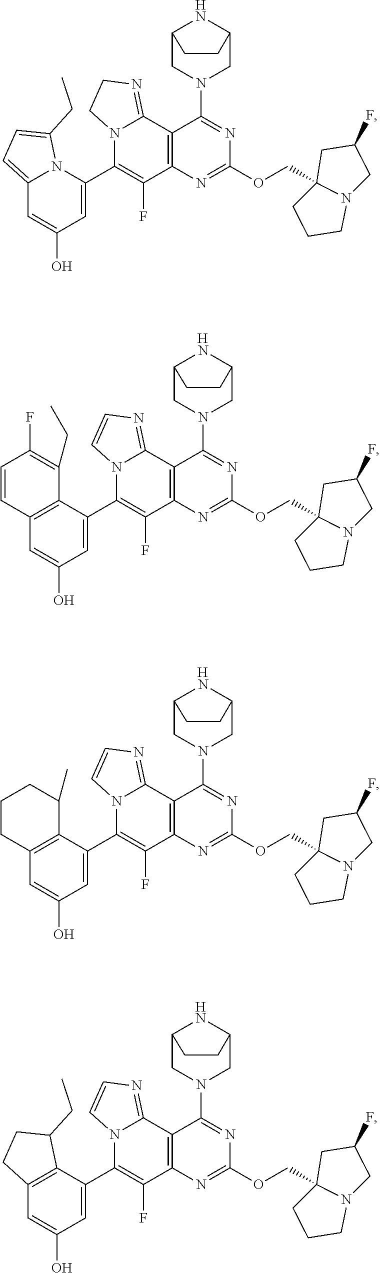

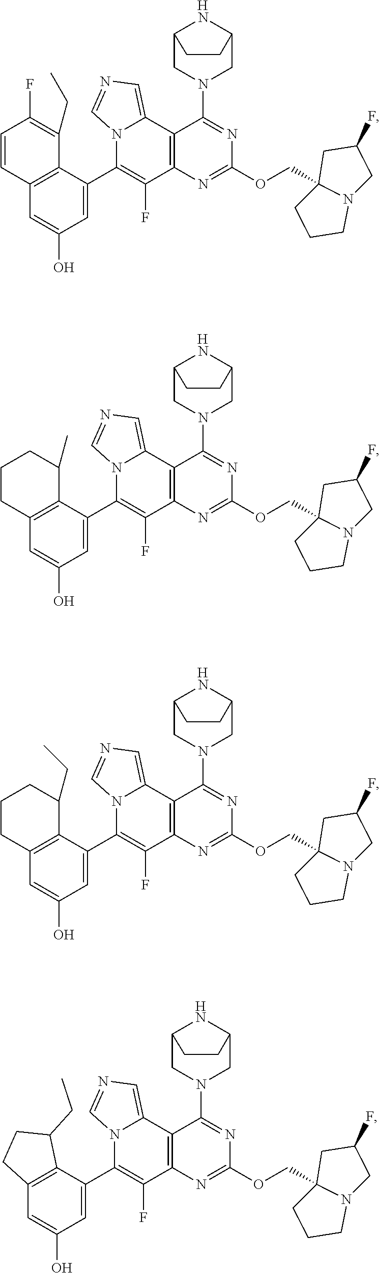

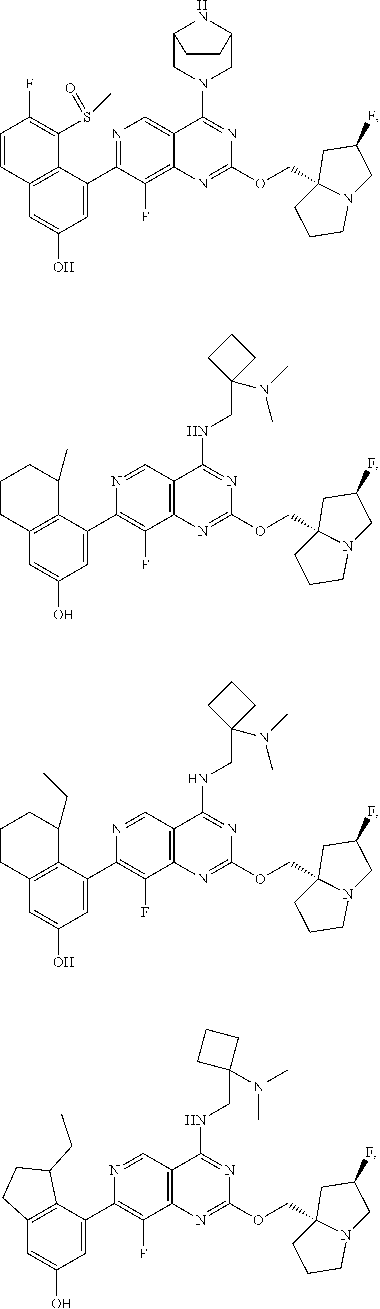

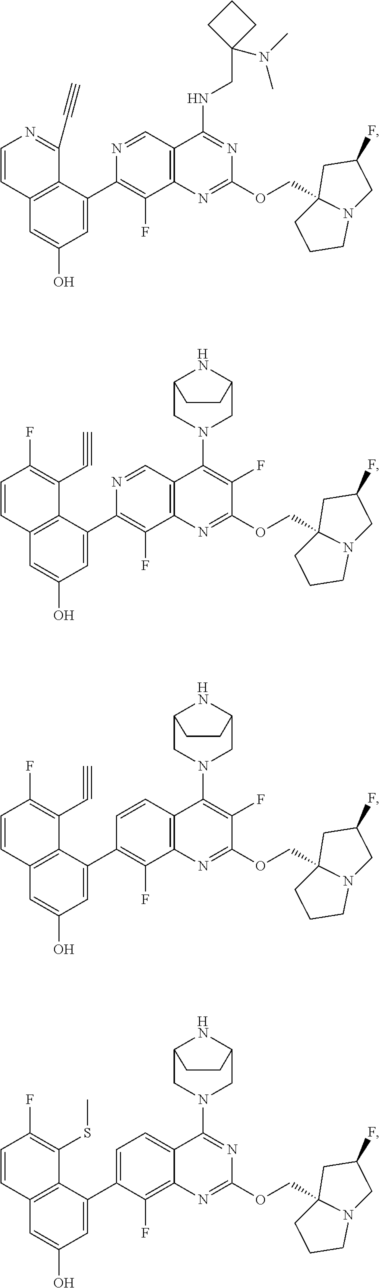

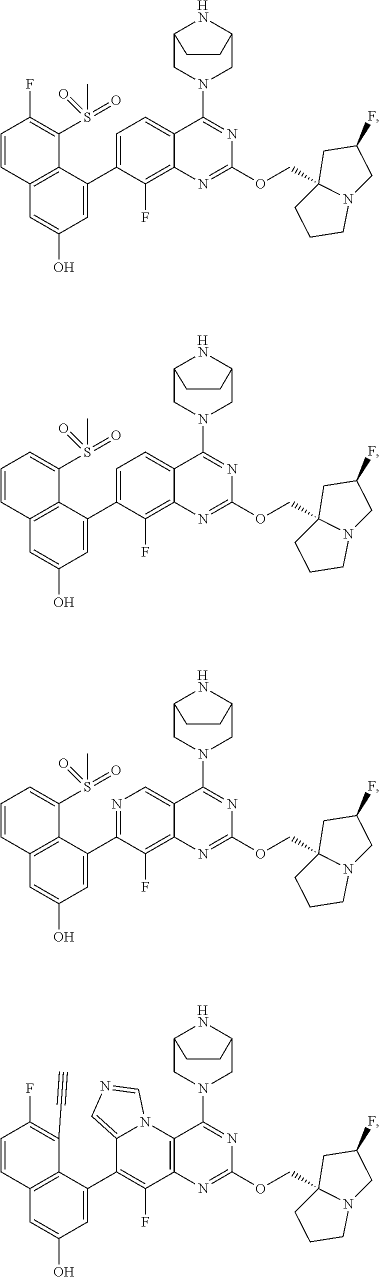

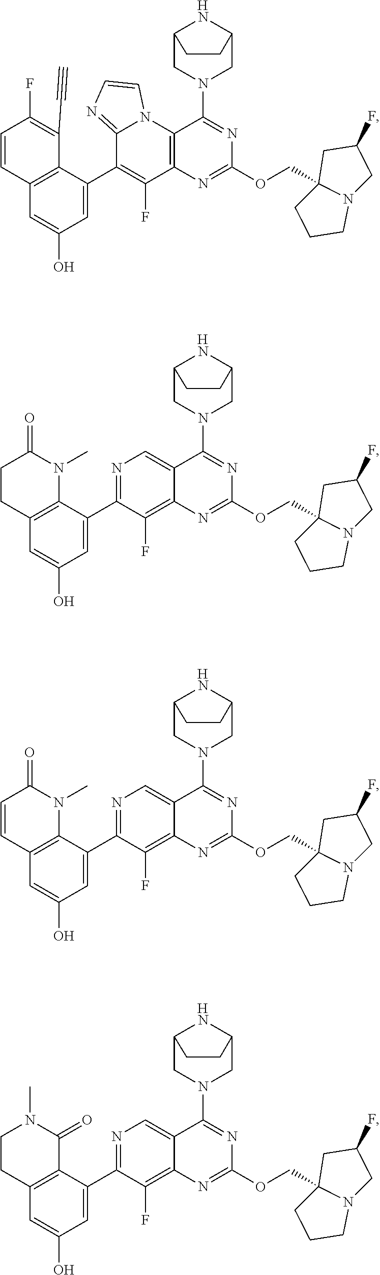

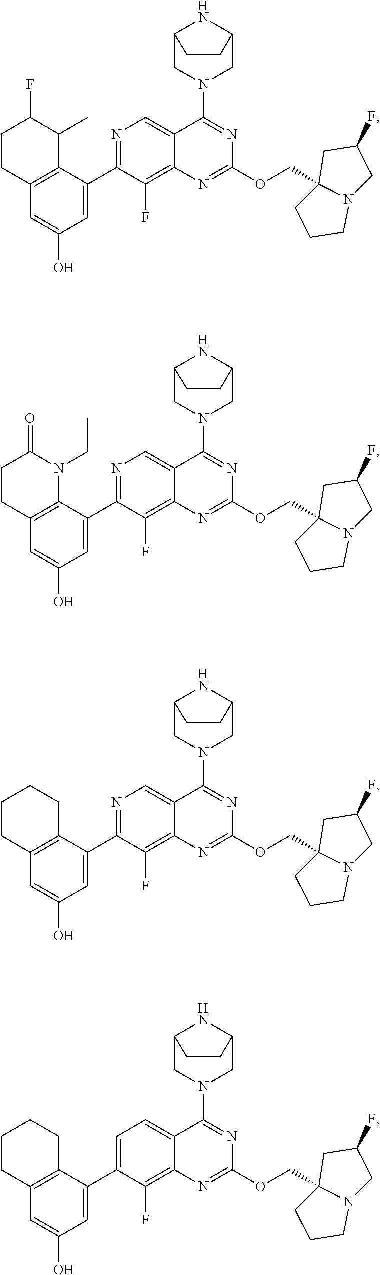

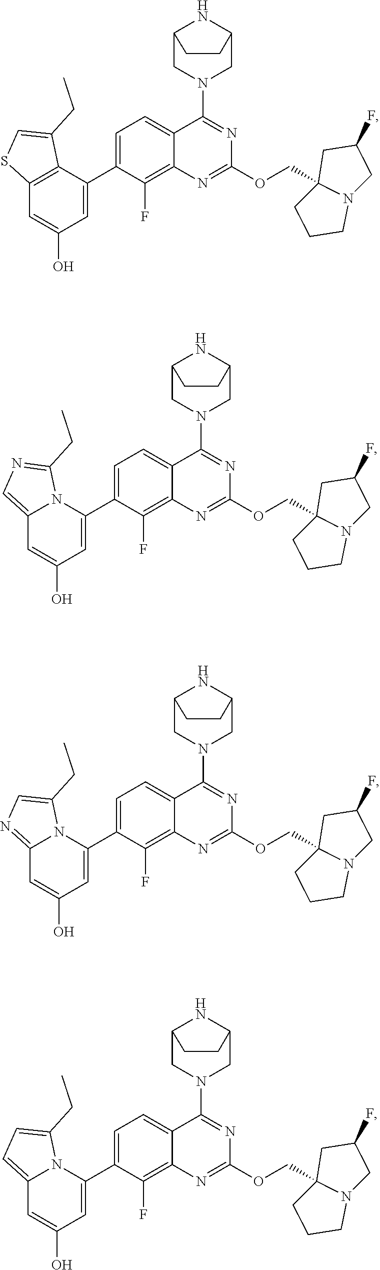 [SMILES: CCc1csc2cc(O)cc(-c3ccc4c(N5CC6CCC(C5)N6)nc(OC[C@@]56CCCN5C[C@H](F)C6)nc4c3F)c12 ; CCc1ncc2cc(O)cc(-c3ccc4c(N5CC6CCC(C5)N6)nc(OC[C@@]56CCCN5C[C@H](F)C6)nc4c3F)n12 ; CCc1cnc2cc(O)cc(-c3ccc4c(N5CC6CCC(C5)N6)nc(OC[C@@]56CCCN5C[C@H](F)C6)nc4c3F)n12 ; CCc1ccc2cc(O)cc(-c3ccc4c(N5CC6CCC(C5)N6)nc(OC[C@@]56CCCN5C[C@H](F)C6)nc4c3F)n12]

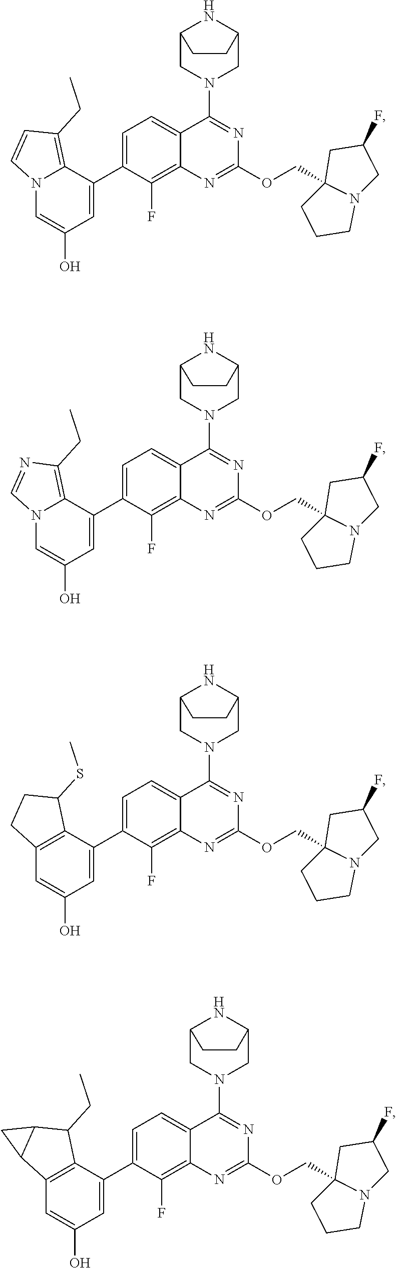 [SMILES: CCc1ccn2cc(O)cc(-c3ccc4c(N5CC6CCC(C5)N6)nc(OC[C@@]56CCCN5C[C@H](F)C6)nc4c3F)c12 ; CCc1ncn2cc(O)cc(-c3ccc4c(N5CC6CCC(C5)N6)nc(OC[C@@]56CCCN5C[C@H](F)C6)nc4c3F)c12 ; CSC1CCc2cc(O)cc(-c3ccc4c(N5CC6CCC(C5)N6)nc(OC[C@@]56CCCN5C[C@H](F)C6)nc4c3F)c21 ; CCC1c2c(-c3ccc4c(N5CC6CCC(C5)N6)nc(OC[C@@]56CCCN5C[C@H](F)C6)nc4c3F)cc(O)cc2C2CC21]

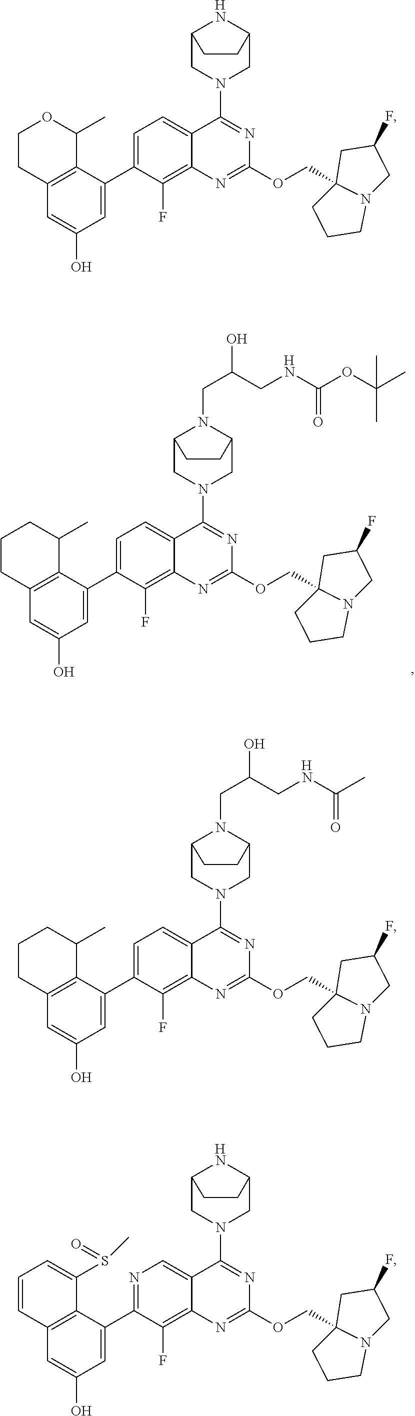

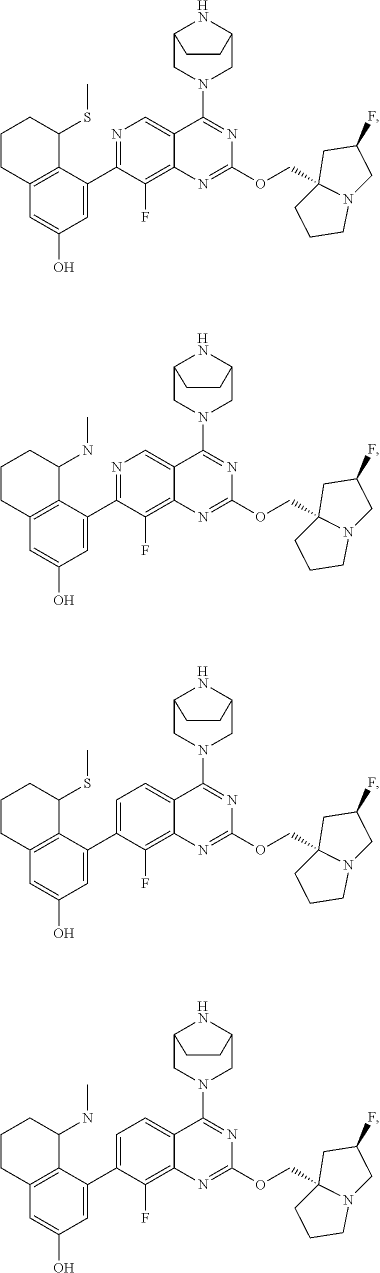

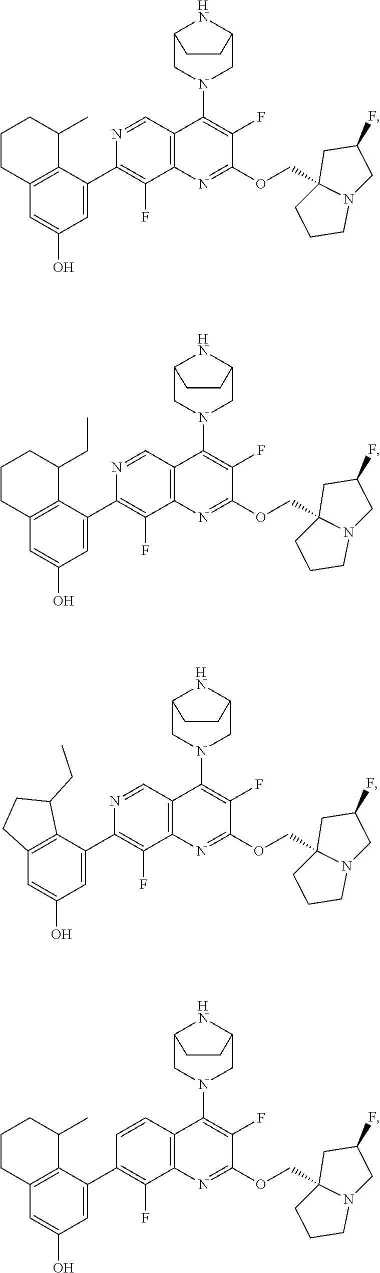

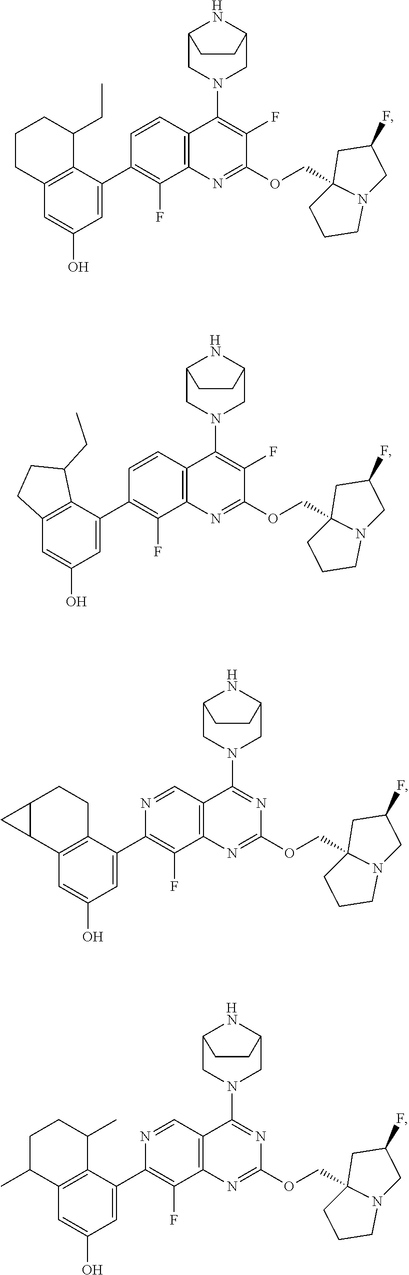

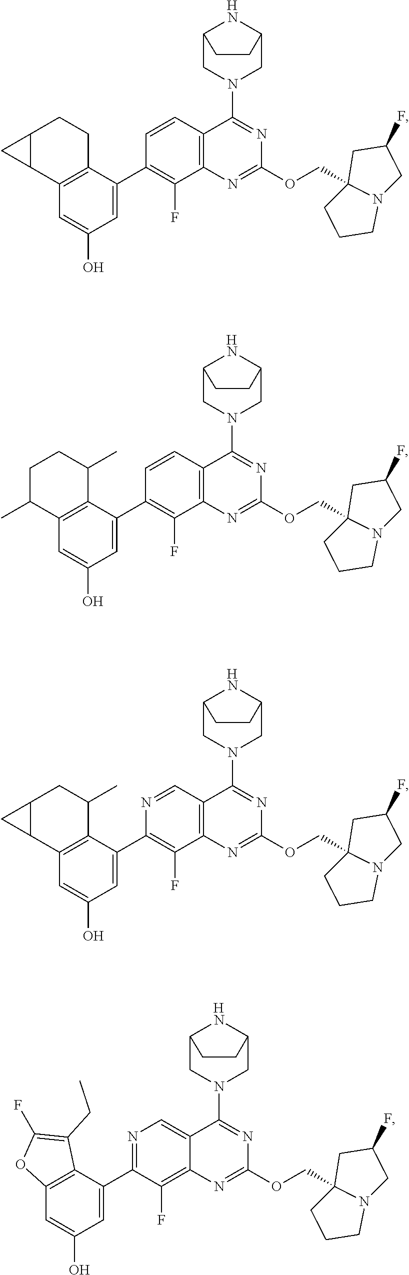

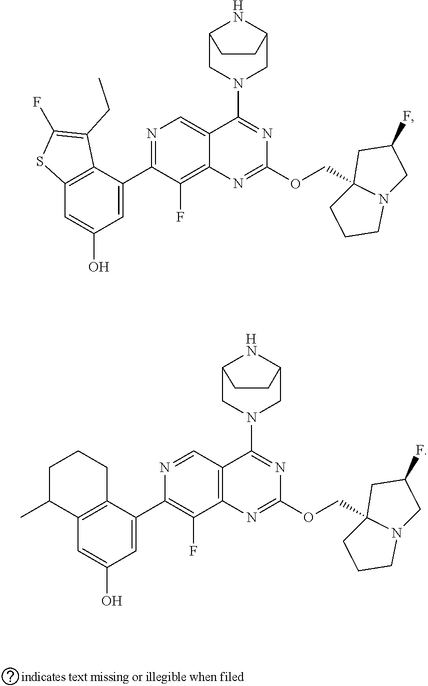

In another aspect of the present invention, there is provided an exemplary method for preparing the compound of formula (I) and (II) or a pharmaceutically acceptable salt, tautomer or stereoisomer thereof:

### Preparation Method 1:

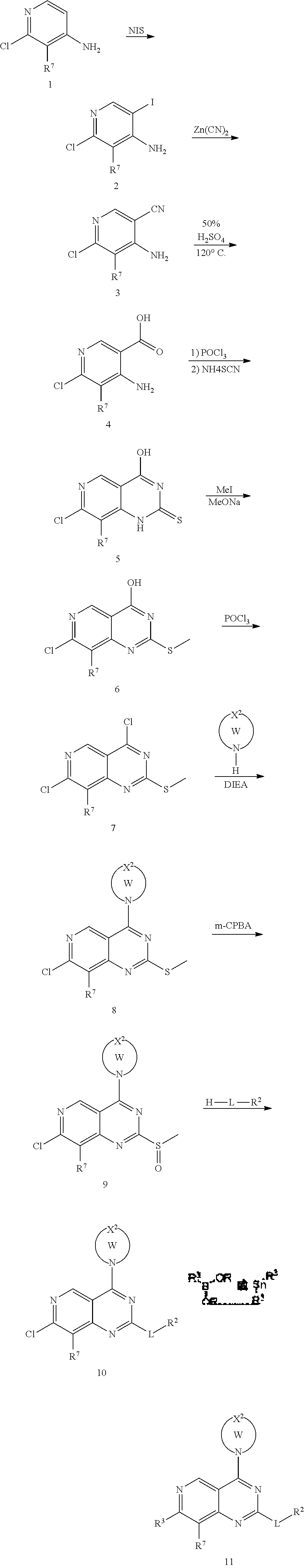 [0065] wherein R¹, R², R³, L, and W are as defined hereinbefore. Compound 1 was iodinated with NIS in the presence of p-toluenesulfonic acid to give compound 2. Compound 3 was obtained by the reaction of compound 2 with Zn (CN) 2 under palladium catalysis (such as Pd (PPh₃)₄), Compound 3 undergoes hydrolysis reaction by heating in 50% H₂SO₄ to 120° C. to obtain compound 4. Compound 4 first reacts with POCl₃ to obtain acyl chloride, and then cyclizes with NH₄SCN to obtain compound 5. Compound 5 undergoes sulfur methylation under sodium methoxide conditions to obtain compound 6. Compound 6 reacts with POCl₃ to obtain compound 7. Compound 7 undergoes a substitution reaction under alkaline conditions (such as triethylamine, diisopropylethylamine, etc.) to obtain compound 8, Compound 8 undergoes an oxidation reaction under the conditions of oxidants such as m-chloroperoxybenzoic acid to obtain the intermediate sulfoxide 9, Compound 9 undergoes a substitution reaction under alkaline conditions (triethylamine, sodium hydride, sodium tert butanol, etc.) to obtain the target intermediate 10, Compound 10 reacts with aryl borate esters or aryltin reagents by Suzuki Coupling or Stille reaction to give the compound (I), wherein, when X² is N-PG, the protective group (PG) is removed by hydrogenation or acid to obtain the compound (I).

### Preparation Method 2:

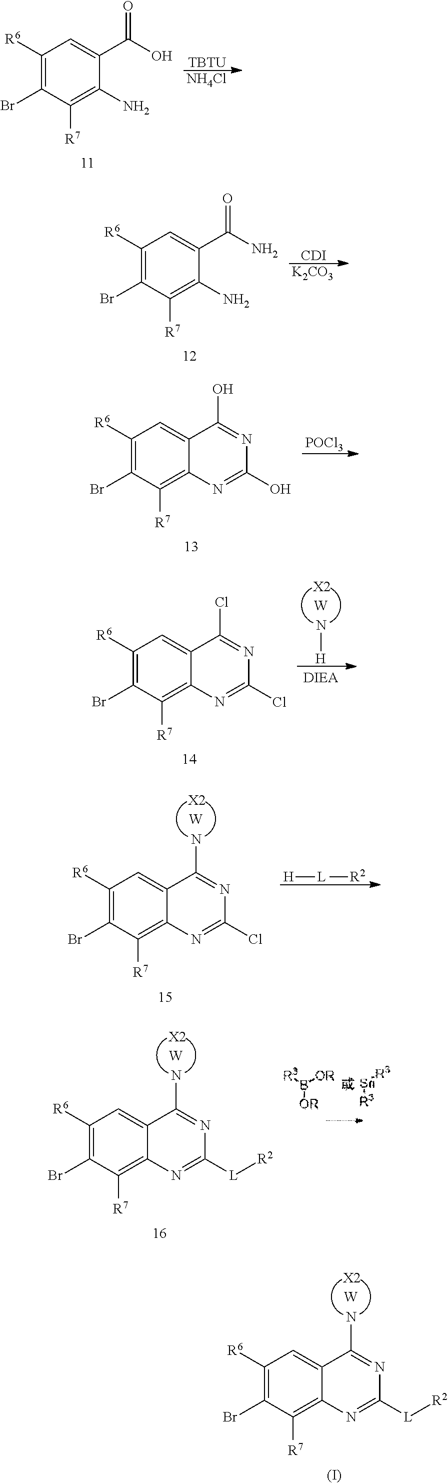

Compound 11 undergoes a condensation reaction with NH₄Cl under the conditions of a condensation agent (such as TBTU) to obtain compound 12, Compound 12 undergoes cyclization reaction with CDI under alkaline conditions (such as K₂CO₃) to obtain compound 13, Compound 13 reacts with POCl₃ to obtain dichloride 14, Compound 14 undergoes a substitution reaction under alkaline conditions (such as triethylamine, diisopropylethylamine, etc.) to obtain compound 15, Compound 15 undergoes a substitution reaction under alkaline conditions (triethylamine, sodium hydride, sodium tert butanol, etc.) to obtain the target intermediate 16, Compound 16 undergoes Suzuki Coupling reaction or Still reaction with aryl borate ester or aryl tin reagent to obtain a compound of formula (I); wherein, when X² is N-PG, the protective group (PG) is removed by hydrogenation or acid to obtain the compound (I).

### Preparation Method 3:

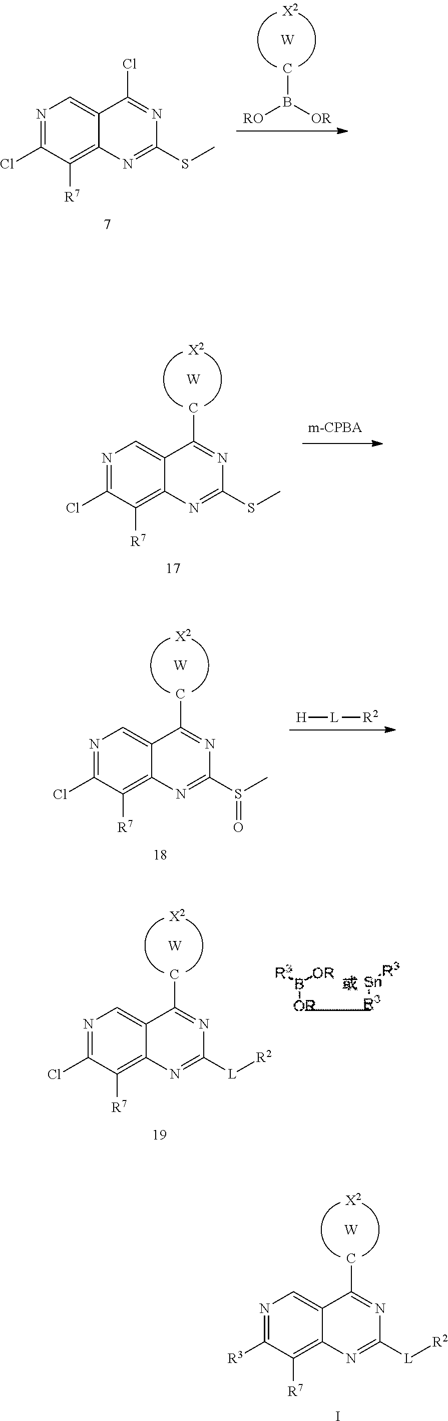

Compound 7 undergoes Suzuki Coupling reaction with borate ester reagent to obtain compound 17, compound 17 undergoes an oxidation reaction under the conditions of oxidants (such as m-chloroperoxybenzoic acid) to obtain the intermediate sulfoxide 18, compound 18 undergoes a substitution reaction under alkaline conditions (triethylamine, sodium hydride, sodium tert butanol, etc.) to obtain the target intermediate 19, compound 19 undergoes Suzuki Coupling reaction or Still reaction with aryl borate ester or aryl tin reagent to obtain a compound of formula (I); wherein, when X² is N-PG, the protective group (PG) is removed by hydrogenation or acid to obtain the compound (I).

### Preparation Method 4:

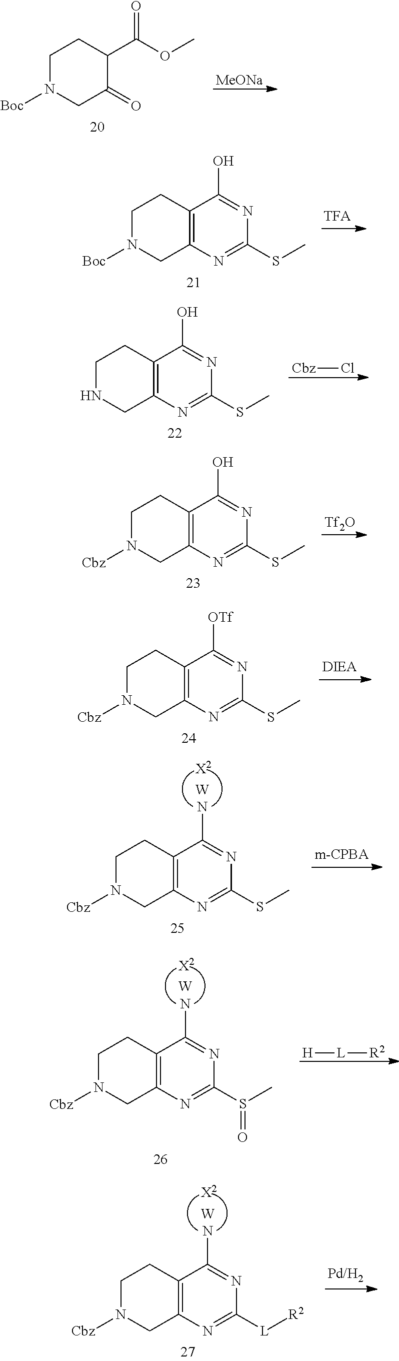

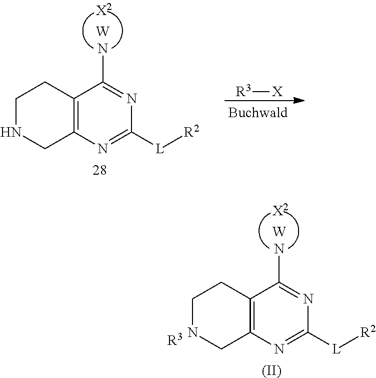

Compound 20 undergoes thiomethylation with thiourea under alkaline conditions (MeONa et al.) to obtain compound 21, compound 20 undergoes thiomethylation with thiourea under alkaline conditions (MeONa et al.) to obtain compound 21, compound 21 is obtained by removing the protective group under acidic conditions to obtain compound 22, compound 22 reacts with Cbz-Cl under alkaline conditions (TEA, DIEA, etc.) to obtain compound 23, compound 23 reacts with trifluoromethanesulfonic anhydride under alkaline conditions (TEA, DIEA, etc.) to obtain compound 24, compound 24 undergoes a substitution reaction under alkaline conditions (such as triethylamine, diisopropylethylamine, etc.) to obtain compound 25, compound 25 undergoes an oxidation reaction under the conditions of oxidants such as m-chloroperoxybenzoic acid to obtain the intermediate sulfoxide 26, compound 26 undergoes a substitution reaction under alkaline conditions (triethylamine, sodium hydride, sodium tert butanol, etc.) to obtain the target intermediate 27, Compound 27 is deprotected under palladium catalyzed hydrogenation to obtain compound 28, Compound 28 undergoes Buchwald reaction with aryl halides to obtain compound (II), wherein, when X² is N-PG, the protective group (PG) is removed by hydrogenation or acid to obtain the compound (II).

### Preparation Method 5:

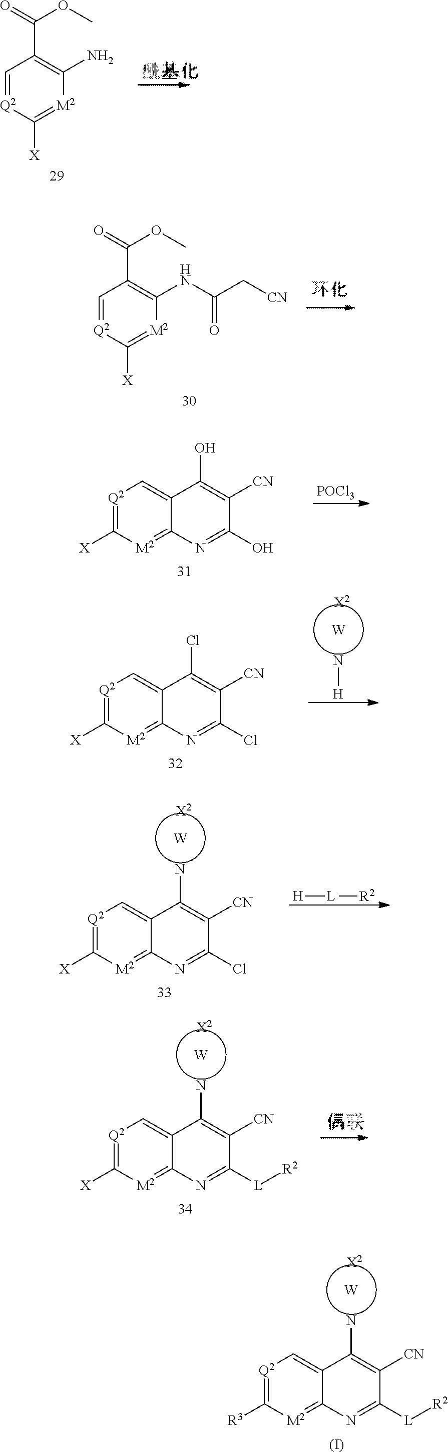

Compound 29 undergoes acylation with cyanoacetic acid or cyanoacetyl chloride to obtain compound 30, compound 30 undergoes cyclization reaction under alkaline conditions (MeONa, t-BuONa, NaH, etc.) to obtain compound 31, compound 31 reacts with POCl3 to obtain dichloride 32, compound 32 undergoes a substitution reaction under alkaline conditions (such as triethylamine, diisopropylethylamine, etc.) to obtain compound 33, Compound 33 undergoes a substitution reaction under alkaline conditions (triethylamine, sodium hydride, sodium tert butanol, etc.) to obtain the target intermediate 34, compound 34 undergoes Suzuki Coupling reaction or Still reaction with aryl borate ester or aryl tin reagent to obtain a compound of formula (I); wherein, when X² is N-PG, the protective group (PG) is removed by hydrogenation or acid to obtain the compound (II).

Other general synthesis methods are provided in the examples. It would be obvious to those of ordinary skill in the art that the compound of formula (I) and (II) can be prepared according to one or more methods or in other means known in the art. Obviously, in general, when following the general route described herein, it is necessary to use diversely substituted starting materials and/or protecting groups to obtain the desired compounds. Various substituents can also be added at different points in the synthesis route to prepare the desired compounds.

The present invention relates to a pharmaceutical composition of the compound of formula (I) or a pharmaceutically acceptable salt, prodrug and solvate thereof.

In yet another aspect of the present invention, there is provided a method of using the compound or pharmaceutical composition of the present invention to treat a disease condition, including but not limited to conditions (such as cancer) associated with G12 KRAS, HRAS or NRAS mutations. The cancer is pancreatic cancer, lung cancer, colorectal cancer, etc., which are mediated by G12D mutation.

The compounds of formula (I) and (II) of the invention have good physical and chemical properties and safety toxicity parameters, and can be used for the treatment of cancer and inflammation in mammals.

In other examples, a method for inhibiting the proliferation of a cell population is further provided, which comprises bringing the cell population into contact with any one of the compounds with structure (I) and (II).

Other embodiments relate to a pharmaceutical composition. The pharmaceutical composition comprises any one (or more) of the aforementioned compounds and a pharmaceutically acceptable carrier.

In some embodiments, the pharmaceutical composition is formulated for oral administration. In other embodiments, the pharmaceutical composition is formulated for injection. In more embodiments, the pharmaceutical composition comprises the compound disclosed herein and another therapeutic agent (e.g., an anticancer agent). Non-limiting examples of such therapeutic agents are described hereinafter.

Suitable routes of administration include but are not limited to oral, intravenous, rectal, aerosol, parenteral, ocular, pulmonary, mucosal, percutaneous, vaginal, auricular, nasal and local administrations. In addition, by way of example only, parenteral delivery includes intramuscular, subcutaneous, intravenous, and intramedullary injections, as well as intrathecal, direct intraventricular, intraperitoneal, endolymphangial and intranasal injections.

### DETAILED DESCRIPTION OF EMBODIMENTS

Unless otherwise indicated, the entire disclosure of the present invention is defined by the following terms:

The term “prodrug” refers to any derivative that can be converted into the corresponding active pharmaceutical compound in an organism. The prodrug of the compound described herein can easily undergo chemical changes under physiological conditions and is thus transformed into the compound of the present invention. In addition, the prodrug can be converted into the compound of the present invention in vivo by a chemical or biochemical method.

Unless otherwise specified, the term “pharmaceutically acceptable salt” includes salts of acidic groups (e.g., but not limited to, potassium salt, sodium salt, magnesium salt, calcium salt, etc.) or salts of basic groups (e.g., but not limited to, formate, acetate, citrate, tartrate, methanesulfonate, malate or sulfate, hydrochloride, phosphate, nitrate, and carbonate) that can be present in the compound of the present invention.

The term “solvate” refers to a complex molecular compound formed by solute molecules or ions attracting adjacent solvent molecules via intermolecular forces such as Coulomb force, van der Waals force, charge transfer force and hydrogen bond in a solution. In one embodiment, the solvent is water, that is, the compound of the present invention forms a hydrate.

The compound of the present invention or the pharmaceutically acceptable salt thereof may contain one or more asymmetric centers, and can thus produce enantiomers, diastereomers and other stereoisomeric forms. As for the absolute stereochemical configuration of amino acids, it is defined as (R)- or (S)-configuration or as (D)- or (L)-configuration. The present invention is intended to include all such possible isomers, as well as racemic and optically pure forms thereof. Optically active (+) and (−), (R)- and (S)-, or (D)- and (L)-isomers can be obtained by chiral synthesis or chiral preparation, or by resolution using conventional techniques such as chromatography and fractional crystallization. Conventional techniques for preparing/separating individual enantiomers include chiral synthesis from suitable optically pure precursors and resolution of racemates (or racemates of salts or derivatives) using, for example, chiral high-pressure liquid chromatography (HPLC). The present invention provides pure isomers and isomer mixtures, a preparation method therefor, the use thereof, and compositions comprising same. For the sake of simplicity, it will be referred to as the compound of formula (I) and (II) hereinafter, which refers to both pure optical isomers and, if appropriate, mixtures of isomers at various ratios.

The compound of the present invention may be present in a specific. Unless otherwise specified, the term “tautomer” or “tautomeric form” means that at room temperature, isomers of different functional groups are in dynamic equilibrium and can quickly transform into each other. If tautomers are possible (such as in a solution), the chemical equilibrium of tautomers can be achieved. For example, proton tautomers (also referred to as prototropic tautomers) include mutual transformation by proton migration, such as keto-enol isomerization and imine-enamine isomerization. Valencetautomers include mutual transformation by recombination of some bonding electrons.

The term “alkyl” refers to alkyl selected from straight-chain and branched-chain saturated alkyl groups, which contain 1 to 18 (such as 1 to 12, further 1 to 10, and further 1 to 8 or 1 to 6 or 1 to 4) carbon atoms. Examples of alkyl (i.e. C₁₋₆ alkyl) containing 1 to 6 carbon atoms include but are not limited to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or sec-butyl (“s-Bu”), 1,1-dimethylethyl or tert butyl (“t-Bu”), 1-pentyl, 2-pentyl, 3-pentyl, 2-methyl-2-butyl, 3-methyl-2-butyl 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl.

The term “halogen” in the present invention refers to fluorine (F), chlorine (Cl), bromine (Br) and iodine (I).

The term “halogenated alkyl” in the present invention refers to the alkyl in which one or more of the hydrogen is substituted by one or more halogen atoms, such as fluorine (F), chlorine (Cl), bromine (Br) and iodine (I). Examples of halogenated alkyl include halogenated C₁₋₈ alkyl, halogenated C₁₋₆ alkyl or halogenated C₁₋₄ alkyl, but are not limited to —CF₃, —CH₂Cl, —CH₂CF₃, —CCl₂, CF₃, etc.

The term “alkenyl” in the present invention refers to alkyl selected from straight-chain and branched-chain alkyl, which contain at least one C═C double bond and 2 to 18 (such as 2 to 8, further as 2 to 6) carbon atoms. Examples of alkenyl such as C₂₋₆ alkenyl include, but are not limited to, vinyl (ethenyl or vinyl), propyl-1-enyl, propyl-2-enyl, 2-methylpropyl-1-enyl, butyl-1-enyl, butyl-2-enyl, butyl-3-enyl, butyl-1,3-dienyl, 2-methyl butyl-1,3-dienyl, hexyl-1-enyl, hexyl-2-enyl, hexyl-3-enyl, hexyl-4-enyl and hexyl-1,3-dienyl.

The term “alkynyl” in the present invention refers to alkyl selected from straight-chain and branched-chain alkyl groups, which contain at least one C≡C triple bond and 2 to 18 carbon atoms (such as 2 to 8, and further to 2 to 6). Examples of alkynyl groups such as C₂₋₆ alkynyl include, but are not limited to, ethynyl, 1-propargyl, 2-propargyl (propargyl), 1-butynyl, 2-butynyl and 3-butynyl.

The term “alkoxy” in the present invention refers to the alkyl as defined above, which is bonded to oxygen, and is represented by —O alkyl. Examples of alkoxy groups such as C₁₋₆ alkoxy or C₁₋₄ alkoxy include, but are not limited to, methoxy, ethoxy, isopropoxy, propoxy, n-butoxy, tert-butoxy, pentoxy and hexoxy, etc.

The term “cycloalkyl” in the present invention refers to the alkyl group selected from saturated and partially unsaturated cyclic hydrocarbons, which contains monocyclic and polycyclic (such as bicyclic and tricyclic) groups. For example, the cycloalkyl group can contain 3 to 12 carbon atoms (such as 3 to 10, further as 3 to 8, further as 3 to 6, 3 to 5, or 3 to 4). Even further, for example, the cycloalkyl group can be selected from a monocyclic group containing 3 to 12 (such as 3 to 10, further such as 3 to 8, 3 to 6) carbon atoms. Examples of monocycloalkyl groups include cyclopropyl, cyclopentyl, cyclopentyl, 1-cyclopentyl-1-enyl, 1-cyclopentyl-2-enyl, 1-cyclopentyl-3-enyl, cyclohexyl, 1-cyclohexyl-1-enyl, 1-cyclohexyl-2-enyl, 1-cyclohexyl-3-enyl, cyclohexadienyl, cycloheptyl, cyclooctyl, cyclononyl, cyclodecyl, cycloundecyl and cyclododecyl. In particular, examples of saturated monocycloalkyl groups such as C₃₋₈ cycloalkyl groups include, but are not limited to, cyclopropyl, cyclobutyl, cyclopentyl, cyclohexyl, cycloheptyl and cyclooctyl. In a preferred embodiment, the cycloalkyl is a single ring (abbreviated as C₃₋₆ cycloalkyl) containing 3 to 6 carbon atoms, including, but not limited to, cyclopropyl, cyclobutyl, cyclopentyl and cyclohexyl. Examples of bicyclic cycloalkyl groups include those with 7 to 12 ring atoms arranged as bicyclic groups selected from [4,4], [4,5], [5,5], [5,6] or [6,6] ring systems, or bridged bicyclic groups selected from bicyclic [2.2.1] heptane, bicyclic [2.2.2] octane, and bicyclic [3.2.2]nonane. Other examples of bicyclic cycloalkyl groups include those arranged as bicyclic cycloalkyl groups selected from the [5,6] and [6,6] ring systems, such as

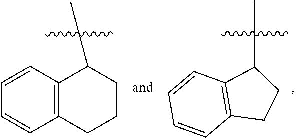 [SMILES: *C1CCCc2ccccc21 ; *C1CCc2ccccc21]

wherein the wavy line represents the attachment point. The ring can be saturated or have at least one double bond (i.e. partially unsaturated), but not completely conjugated and not aromatic, as aromatic groups are defined in this article.

The term ‘aryl’ used alone or in combination with other terms refers to a group selected from:
a. 5-membered and 6-membered carbon ring aromatic ring, such as phenyl;
b. A double-ring system, such as a 7- to 12-membered double-ring system, in which at least one ring is carbon ring and aromatic, such as naphthyl; and
c. A tricyclic system, such as a 10- to 15-membered tricyclic system, in which at least one ring is carbon ring and aromatic, such as fluorenyl.

The terms “aromatic ring” and “aryl” are used interchangeably in the disclosure of the present invention. In some embodiments, monocyclic or bicyclic aromatic rings have 5 to 10 cyclic carbon atoms (i.e., C₅₋₁₀ aryl). Examples of monocyclic or bicyclic aromatic rings include, but are not limited to, phenyl, naphthalene-1-yl, naphthalene-2-yl, anthracene, phenanthrene, etc. In some embodiments, the aromatic ring is a naphthalene ring (naphthalene-1-yl or naphthalene-2-yl) or benzene ring. In some embodiments, the aromatic ring is a benzene ring.

The term “heteroaryl” in the present invention refers to a group selected from:
a. 5, 6 or 7-membered aromatic single ring containing at least one heteroatom, such as 1 to 4 heteroatoms, or in some embodiments containing 1 to 3 heteroatoms, and in some embodiments containing 1 to 2 heteroatoms, those heteroatoms were selected from nitrogen (N), sulfur (S), and oxygen (O) (as one or more ring atoms), with the remaining ring atoms is carbon;
b. 7 to 12-membered bicycles, containing at least one heteroatom, such as 1 to 4 heteroatoms, or in some embodiments 1 to 3 heteroatoms, or in other embodiments 1 or 2 heteroatoms, those heteroatoms were selected from N, O and S (as one or more ring atoms), the remaining ring atoms are carbon, and at least one of the rings is aromatic and there is at least one heteroatom in the aromatic ring; and
c. 11 to 14-membered three ring, containing at least one heteroatom, such as 1 to 4 heteroatoms, or in some embodiments 1 to 3 heteroatoms, or in other embodiments 1 or 2 heteroatoms, those heteroatoms were selected from N, O, and S (as one or more ring atoms), with the remaining ring atoms are carbon, and at least one of the rings is aromatic and there is at least one heteroatom in the aromatic ring.

In a preferred embodiment, the heteroaryl is a 5 to 6-membered heteroaryl containing one nitrogen atom and 0 or one additional heteroatom selected from N, O and S, including, but not limited to, pyridyl, isoxazolyl and oxazolyl.

When the total number of S and O atoms in the heteroaryl exceeds 1, those heteroatoms are not adjacent to each other. In some embodiments, the total number of S and O atoms in the heteroaryl is not more than 2. In some embodiments, the total number of S and O atoms in the aromatic heterocyclic ring is not greater than 1. When heteroaryl contain more than one heteroatom ring member, heteroatoms can be the same or different. Nitrogen atoms in one or more rings of heteroaryl groups can be oxidized to form N-oxides.

The terms “aromatic heterocycles” and “heteroaryl” are used interchangeably in the disclosure of this article. In some embodiments, monocyclic or bicyclic aromatic heterocycles have 5, 6, 7, 8, 9 or 10 ring members, of which 1, 2, 3 or 4 heteroatom ring members are independently selected from nitrogen (N), sulfur (S) and oxygen (O), and the remaining ring members are carbon. In some embodiments, monocyclic or bicyclic aromatic heterocycles are monocycles or bicycles containing 1 or 2 heteroatom ring members independently selected from nitrogen (N), sulfur (S) and oxygen (O). In some embodiments, monocyclic or bicyclic aromatic heterocycles are 5- to 6-membered heteroaryl rings, which are single rings and have 1 or 2 heteroatom ring members independently selected from nitrogen (N), sulfur (S) and oxygen (O). In some embodiments, monocyclic or bicyclic aromatic heterocycles are 8- to 10-membered heteroaryl rings that are bicyclic and have 1 or 2 heteroatom ring members independently selected from nitrogen, sulfur, and oxygen.

Examples of heteroaromatic or mono or bicyclic aromatic heterocycles include but are not limited to (numbering from the designated priority 1 connection position) pyridinyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazolopyridyl, Isoxazolyl, oxazolyl, thiazolyl, isothiazolyl, thiadiazolyl (such as 1,2,3-thiadiazolyl, 1,2,4-thiadiazolyl or 1,3,4-thiadiazolyl), tetrazolyl, thiophenyl (e.g. thiophene-2-yl, thiophene-3-yl), triazinyl, benzothiophenyl, furyl (furyl or furanyl), benzofuranyl, benzimidazolyl, indolyl, isoindolyl, dihydroindolyl, oxadiazolyl (such as 1,2,3-oxadiazolyl, 1,2,4-oxadiazolyl or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), Quinolinyl, isoquinolinyl, pyrazolyl, pyrrolopyridyl (such as 1H-pyrrolo[2,3-b]pyridin-5-yl), pyrazolpyridyl (such as 1H-pyrrolo[3,4-b]pyridin-5-yl), benzoxazolyl (such as benzo[d]oxazole-6-yl), pteridinyl, purinyl, 1-oxa-2,3-diazolyl, 1-oxa-2,4-dizolyl, 1-oxa-2,5-diazolyl, 1-oxa-3,4-diazolyl, 1-thia-2,3-diazolyl, 1-thia-2,4-dizolyl, 1-thia-2,5-diazolyl, 1-thia-3,4-diazolyl, furazolyl (such as furoxan-2-yl, furoxan-3-yl), benzofuroxanyl, benzobenzothiazolyl, benzooxazolyl, quinazolinyl, quinoxalinyl, naphthyl, furan pyridyl, benzothiazolyl (such as benzo[d]thiazole-6-yl), indazolyl (such as 1H-indazole-5-yl), and 5,6,7,8-tetrahydroisoquinoline.

The term “heterocyclic” or “heterocyclic” or “heterocyclic base” refers to a ring selected from 3, 4, 5, 6, 7, 8, 9, 10, 11 or 12-membered single ring, bicyclic and tricyclic saturated ring and partially unsaturated ring, it contains at least one carbon atom and at least one heteroatom, such as 1 to 4 heteroatoms, further such as 1 to 3 heteroatoms or further such as 1 or 2 heteroatoms. These heteroatoms are selected from nitrogen (N), sulfur (S), oxygen (O), —SO— or —SO₂ (as one or more ring atoms).

In some embodiments, the heterocyclyl is a 4, 5, 6, 7, or 8-membered single ring with at least one heteroatom selected from N, O, and S. In some preferred embodiments, the heterocyclyl is a 4-, 5-, 6-, 7- or 8-membered saturated ring containing a nitrogen heteroatom. Example heterocyclyl are azetidinyl, pyrrolyl, piperidyl, azepanyl, and azocanyl. In other embodiments, the heterocyclyl is a 5-membered, 6-membered, 7-membered, or 8-membered saturated single ring, which contains one nitrogen atom and one additional heteroatom selected from —NH, —O—, —S—, —SO—, or —SO₂—. The exemplary heterocyclyl is morpholine, morpholinyl, or piperazinyl ring. In some embodiments, the heterocyclyl is a 7- to 12-membered saturated bicyclic ring, which contains a nitrogen atom and 0 or 1 or 2 additional heteroatoms selected from—NH, —O—, —S—, —SO— or —SO₂—. In some preferred embodiments, the heterocyclyl is a double ring bridging ring or a spirocycle.

The term ‘heterocycle’ in this article also refers to a 5-to-7-membered heterocycle that contains at least one heteroatom selected from N, O, and S, fused with a 5, 6, and/or 7-membered cycloalkyl, carbon ring aromatic ring, or heteroaromatic ring, provided that the entire ring structure is non aromatic.

Heterocycles are not heteroaryl groups as defined in this article. In a preferred embodiment, the heterocyclic group is a 5-to-6-membered heterocyclic group consisting of one nitrogen atom and 0 or 1 additional heteroatom selected from N, O, and S, including but not limited to pyrrolyl, dihydropyridinyl, morpholine, morpholinyl, and tetrahydropyranyl.

Examples of heterocycles include, but are not limited to (numbering starting with the connection position designated as priority 1) 1-pyrrolidinyl, 2-pyrrolidinyl, 2,4-imidazolidine, 2,3-pyrrolidinyl, 1-piperidinyl, 2-piperidinyl, 3-piperidinyl, 4-piperidinyl, 2,5-piperazinyl, pyranyl, morpholinyl, morpholinyl, 2-morpholinyl, 3-morpholinyl, oxiranyl, aziridinyl, cyclothioethanyl, azetidinyl, oxetanyl, thietanyl, 1,2-dithietanyl, 1,3-dithietanyl, dihydropyridyl, tetrahydropyridyl, thiomorphinyl, thioxanyl, piperazinyl, homopiperazinyl, homopiperidyl, azepanyl, oxepanyl, thiepanyl, 1,4-oxathianyl, 1,4-dioxoheptanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothiophenyl, dihydropyranyl, dihydrofuranyl, tetrahydrofuranyl, tetrahydrothienyl, tetrahydropyranyl, tetrahydrothiopyranyl, 1-pyrrolizinyl, 2-pyrrolizinyl, 3-pyrrolizinyl, dihydroindolyl, 2H-pyranyl, 4H-pyranyl, 1,4-dioxanyl, 1,3-dioxolanyl, pyrazolidinyl, pyrazolinyl, pyrazolinyl, pyrazolidyl, dithianyl, dithiolanyl, pyrazolidinyl, Imidazolinyl, pyrimidinonyl, 1,1-dioxo-thiomorpholinyl, 3-azabicyclo[3.1.0]hexanyl, 3-azabicyclo[4.1.0]heptyl, azabicyclo[2.2.2]hexanyl, tetrahydro-1H-pyrrolizin-7-yl, tetrahydro-1H-furano[3,4-b]pyrrole-3a-yl, tetrahydro-1H-furano[3,4-c]pyrrole-3a-yl, tetrahydro-1H-furano[3,4-b]pyrrole-6a-yl, octahydrocyclopentano[b]pyrrole-6a-yl, octahydrocyclopentano[c]pyrrole-3a-yl, octahydropentano[b]pyrrole-3a-yl 3-azabicyclic[3.1.0] hexan-1-yl or 2-azabicyclic[3.1.0]hexan-1-yl. The substituted heterocycles also include ring systems partially substituted by one or more oxygenyl, such as piperidinyl-N-oxide, morpholinyl-N-oxide, 1-oxo-1-thiomorpholinyl, and 1,1-dioxo-1-thiomorpholinyl.

In some embodiments, heterocyclyl is non-aromatic fused bicyclic heterocyclyl, such as the fused bicyclic heterocycles listed above, and the following non-aromatic fused bicyclic heterocyclyl.

The term “optional” or “optionally” means that the subsequently described event or condition possibly, but not necessarily, occurs, and the description includes the case where the event or condition occurs and the case where the event or condition does not occur.

The term “substituted” means that any one or more hydrogen atoms on a specific atom are replaced with a substituent, which may include heavy hydrogen and hydrogen variants, as long as the valence state of the specific atom is normal and the substituted compound is stable. When the substituent is oxygen (i.e., ═O), it is meant that two hydrogen atoms are replaced. Substitution with oxygen does not occur on aromatic groups. The term “optionally substituted” refers to either substituted or unsubstituted. Unless otherwise specified, the type and number of substituents may be arbitrary on the basis of being achievable in chemistry.

When any variable (such as R) appears more than once in the composition or structure of a compound, the definition thereof in each case is independent. Therefore, for example, if one group is substituted with 0-2 R, the group can be optionally substituted with at most two R, and R in each case has an independent option. In addition, a combination of substituents and/or variants thereof is allowed only if such a combination produces a stable compound. The term “one or more” disclosed in this article. The following group substituents “include, for example, 1 to 5 substituents (such as 1 to 4, further such as 1, 2 or 3), provided that the valence permits.

Unless otherwise specified, the term “heteroalkyl”, by itself or in combination with another term, represents a stable linear or branched alkyl radical or its composition which consists of a certain number of carbon atoms and at least one heteroatom or heteroatom radical. In some embodiments, the heteroatom is selected from B, O, N, and S, wherein the nitrogen and sulfur atoms are optionally oxidized and the nitrogen heteroatom is optionally quaternized. In other embodiments, the heteroatom radical is selected from —C(═O)O—, —C(═O)—, —C(═S)—, —S(═O), —S(═O)₂—, —C(═O)N(H)—, —N(H)—, —C(═NH)—, —S(═O)₂N(H)—, and —S(═O)N(H)—. In some embodiments, the heteroalkyl is C₁-C₆ heteroalkyl; and in other embodiments, the heteroalkyl is C₁-C₃ heteroalkyl. The heteroatom or heteroatom radical can be located in any internal position of the heteroalkyl, including the position at which the alkyl is connected to the remainder of the molecule, but the terms “alkoxy”, “alkylamino” and “alkylthio” (or thioalkoxy) are customary expressions and refer to alkyl groups that are connected to the remainder of the molecule via an oxygen atom, amino, or a sulfur atom, respectively. Examples of heteroalkyl include, but are not limited to, —OCH₃, —OCH₂CH₃, —OCH₂CH₂CH₃, —OCH₂(CH₃)₂, —CH₂—CH₂—O—CH₃, —NHCH₃, —N(CH₃)₂, —NHCH₂CH₃, —N(CH₃)(CH₂CH₃), —CH₂—CH₂—NH—CH₃, —CH₂—CH₂—N(CH₃)—CH₃, —SCH₃, —SCH₂CH₃, —SCH₂CH₂CH₃, —SCH₂(CH₃)₂, —CH₂—SCH₂—CH₃, —CH₂—CH₂, —S(═O)—CH₃, —CH₂—CH₂—S(═O)₂—CH₃, —CH═CHO—CH₃, —CH₂—CH═N—OCH₃, and —CH═CHNCCH₃)—CH₃. At most two heteroatoms can be continuous, e.g., in —CH₂—NH—OCH₃.

Unless otherwise specified, the termalkyl, alkenyl, alkynyl, cycloalkyl, aryl, heterocyclyl and heteroaryl moieties in the present invention can be each independently substituted by one or more groups selected from the following group: hydroxyl, oxygen, halogen, cyano, nitro, trifluoromethyl, azido, amino, carboxyl, sulfhydryl.

### Synthesis

All suitable solvents commonly used in organic reactions can be used in the following steps of the preparation method of the present invention, e.g., but not limited to, aliphatic and aromatic, optional hydrocarbons or halogenated hydrocarbons (e.g., pentane, hexane, heptane, cyclohexane, petroleum ether, gasoline, volatile oils, benzene, toluene, xylene, dichloromethane, dichloroethane, chloroform, carbon tetrachloride, chlorobenzene and o-dichlorobenzene), aliphatic and aromatic, optional alcohols (e.g., methanol, ethanol, propanol, isopropanol, tert-butanol, and ethylene glycol), ethers (e.g., diethyl ether, dibutyl ether, ethylene glycol dimethyl ether, diethylene glycol dimethyl ether, tetrahydrofuran and dioxane), esters (e.g., methyl acetate or ethyl acetate), nitriles (e.g., acetonitrile or propionitrile), ketones (e.g., acetone and butanone), amides (e.g., dimethylformamide, dimethylacetamide, and N-methylpyrrolidone), dimethyl sulfoxide, tetramethylene sulfone, hexamethylphosphoryl triamine, N,N-dimethylpropylene urea (DMPU), etc.

The following abbreviations are used in the present invention: DCM stands for dichloromethane; CHCl₃ stands for trichloromethane; EA stands for ethyl acetate; THF stands for tetrahydrofuran; MeCN stands for acetonitrile; MeOH stands for methanol; EtOH stands for ethanol; i-PrOH stands for isopropanol; PE stands for petroleum ether; toulene stands for methylbenzene; DMSO stands for dimethyl sulfoxide; DMF stands for N,N-dimethylformamide; DMA stands for N,N-dimethylacetamide; CDCl₃ stands for deuterated chloroform; D₂O stands for heavy water; (CD₃)₂SO stands for deuterated DMSO; CD₃OD stands for deuterated methanol; CuI stands for cuprous iodide; DIPEA stands for diisopropylethylamine; TEA stands for triethylamine; K₂CO₃ stands for potassium carbonate; Cs₂CO₃ stands for cesium carbonate; Na₂CO₃ stands for sodium carbonate; NaHCO₃ stands for sodium bicarbonate; NaOH stands for sodium hydroxide; KOH stands for potassium hydroxide; LiHMDS stands for potassium hexamethyldisilazide; CDI stands for 1,1′-carbonyl imidazole; MS stands for mass spectrometry; NMR stands for nuclear magnetic resonance; TFA stands for trifluoroacetic acid; BINAP stands for (2R,3S)-2,2′-diphenylphosphine-1,1′-binaphthyl; BOC stands for tert-butoxycarbonyl; Cbz stands for benzyloxycarbonyl; DBU stands for bicyclo-1,5-diaza-5-undecene; DCC stands for 1,3-dicyclohexylcarbodiimide; DCE stands for 1,2-dichloroethane; DMAP stands for 4-dimethylaminopyridine; dppf stands for bis(diphenylphosphino)ferrocene; LiAlH4 stands for lithium aluminium hydride; LDA stands for lithium diisopropylamide; m-CPBA stands for m-chloroperoxybenzoic acid; MTM stands for dimethyl sulfide; NBS stands for N-bromosuccinimide; NCS stands for N-chlorosuccinimide; NIS stands for N-iodosuccinimide; PCC stands for pyridinium dichromate; TBAF stands for tetrabutylamine fluoride; THP stands for tetrahydropyranyl; TMEDA stands for tetramethylethylene diamine; TMS stands for trimethylsilyl; TMP stands for 2,2,6,6-tetramethylpiperidine; Ts stands for p-toluenesulfonyl; Pd(PPh₃)₄ stands for tetrakis(triphenylphosphine)palladium; PdCl₂(dppf) stands for 1,1′-bis(diphenylphosphino)ferrocene palladium(II) dichloride; Pd₂(dba)₃ stands for tris(dibenzylideneacetone)dipalladium; HOBT stands for 1-hydroxybenzotriazole; HATU stands for 2-(7-oxidobenzotriazole)-N,N,N′,N′-tetramethyluronium hexafluorophosphate; TBTU stands for O-benzotriazole-N,N,N′,N′-tetramethyluronium tetrafluoroborate; Tf₂O stands for trifluoroacetic anhydride; Pd(OAc)₂ stands for palladium diacetate; RuPhos stands for 2-dicyclohexylphosphino-2′,6′-diisopropoxy-1,1′-biphenyl; Pd(PPh₃)₂Cl₂ stands for bis(triphenylphosphine)palladium(II) dichloride; Sphos stands for 3,2-dicyclohexylphosphino-2′,6′-dimethoxybiphenyl; XantPhos stands for 4,5-bis(diphenylphosphino)-9,9-dimethylxanthene; MeONa stands for sodium methoxide; n-BuLi stands for n-butyl lithium; t-BuONa stands for sodium tert-butoxide; t-BuOK stands for potassium tert-butoxide; KSCN stands for potassium thiocyanate; CuBr stands for cuprous bromide; NaNO₂ stands for sodium nitrite; urea stands for carbamide; POCl₃ stands for phosphorus oxychloride; BBr₃ stands for boron tribromide; NH₄Cl stands for ammonium chloride; Mel stands for iodomethane; NMP stands for N-methylpyrrolidone; K₃PO₄ stands for potassium phosphate; column chromatography stands for column chromatography separation; Ac stands for acetyl; Bn stands for benzyl; Fmoc stands for fluorenylmethyloxycarbonyl; Cy stands for cyclohexyl; Tf stands for trifluoromethylsulfonyl; and PDC stands for pyridine dichromate.

### Synthesis Examples

### Preparation of Intermediate

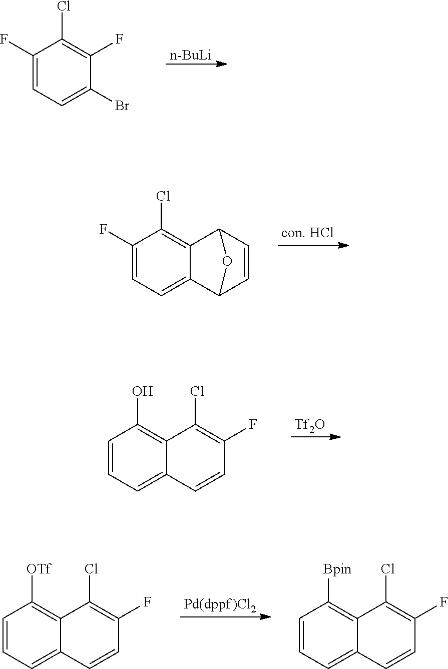

### Step 1: Synthesis of 5-chloro-6-fluoro-1,4-dihydro-1,4-epoxynaphthalene

The compound 1-bromo-3-chloro-2,4-difluorobenzene (5.0 g, 21.98 mmol) and furan (2.99 g, 43.97 mmol) were dissolved in anhydrous toluene (50 mL); in a nitrogen atmosphere, after the reaction liquid was cooled to −15° C., n-BuLi (10.6 mL, 26.38 mmol) was added dropwise to the reaction liquid, and after the dropwise addition was complete, the reaction liquid was slowly heated to room temperature and reacted under stirring for 12 h; and after the reaction was complete, the reaction was quenched with saturated ammonium chloride and extracted with methyl tert-butyl ether, and the organic phase was washed with a saturated aqueous NaCl solution, dried with anhydrous sodium sulfate, and concentrated to obtain a brown oil, which was directly used for the next step. (4.3 g, yield: 100%).

### Step 2: Synthesis of 8-chloro-7-fluoronaphthalen-1-ol

The crude compound obtained from the previous step (synthesis of 8-chloro-7-fluoronaphthalen-1-ol), i.e., 5-chloro-6-fluoro-1,4-dihydro-1,4-epoxynaphthalene, (4.3 g, 21.98 mmol) was dissolved in ethanol (10 ml) and concentrated hydrochloric acid (8 mL), and the mixture was heated to 80° C. and reacted under stirring for 4 h. After the reaction was complete, the reaction liquid was cooled to room temperature, diluted with water, extracted with ethyl acetate, the organic phase was washed with a saturated aqueous NaCl solution, dried with anhydrous sodium sulfate, and concentrated to obtain a brown oil, the brown oil was placed in a refrigerator for 24 h to precipitate out a solid, which was diluted with petroleum ether, filtered, washed with petroleum ether, and dried to obtain an off-white solid. 1.3 g, yield: 30%. 1H NMR (400 MHz, CDCl3) δ 7.91 (s, 1H), 7.75 (dd, J=9.1, 5.6 Hz, 1H), 7.44-7.34 (m, 2H), 7.30 (d, J=8.7 Hz, 1H), 7.08 (d, J=7.1 Hz, 1H).

### Step 3: Synthesis of 8-chloro-7-fluoronaphthalen-1-yl trifluoromethanesulfonate

The compound 8-chloro-7-fluoronaphthalen-1-ol (1.0 g, 5.08 mmol) was dissolved in anhydrous dichloromethane (10 mL), DIEA (3.94 g, 30.51 mmol) and a molecular sieve (1 g) were added, the mixture was stirred for 10 min at room temperature and then cooled to −40° C., and trifluoromethanesulfonic anhydride (1.86 g, 6.61 mmol) was added dropwise to the reaction liquid; after a stirred reaction was carried out for 20 min, the reaction was quenched with water and extracted with dichloromethane, and the organic phase was washed with a saturated aqueous NaCl solution, dried with anhydrous sodium sulfate, concentrated and separated by column chromatography to obtain a yellow solid. (1.65 g, yield: 98.8%). 1H NMR (400 MHz, CDCl3) δ 7.90 (d, J=8.1 Hz, 1H), 7.84 (dd, J=9.0, 5.4 Hz, 1H), 7.59 (d, J=7.7 Hz, 1H), 7.51 (s, 1H), 7.44 (s, 1H).

### Step 4: Synthesis of 2-(8-chloro-7-fluoronaphthalen-1-yl)-4,4,5,5-tetramethyl-1,3,2-dioxaborane

The compound 8-chloro-7-fluoronaphthalen-1-yl trifluoromethanesulfonate (1.65 g, 5.02 mmol) and pinacol borate (2.53 g, 10.04 mmol) were dissolved in anhydrous DMF (20 mL), potassium acetate (2.44 g, 24.85 mmol) and Pd(dppf)Cl₂ (366 mg, 0.50 mmol) were added, and after displacement with nitrogen, a stirred reaction was carried out in a nitrogen atmosphere for 12 h. After the reaction was complete, the reaction liquid was cooled to room temperature, diluted with water and extracted with ethyl acetate, and the organic phase was washed with a saturated aqueous NaCl solution, dried with anhydrous sodium sulfate, concentrated and separated by column chromatography to obtain an off-white solid. (1.25 g, yield: 82%). 1H NMR (400 MHz, CDCl3) δ 7.83 (t, J=10.4 Hz, 1H), 7.75 (dd, J=9.0, 5.5 Hz, 1H), 7.70 (d, J=6.8 Hz, 1H), 7.50-7.44 (m, 1H), 7.32 (t, J=8.7 Hz, 1H), 1.45 (s, 12H).

### Synthesis of triisopropyl((6-(methoxymethoxy)-8-(4,4,5,5-tetramethyl-1,3,2-dioxaborolan-2-yl)naphthalen-1-yl)ethynyl)silane

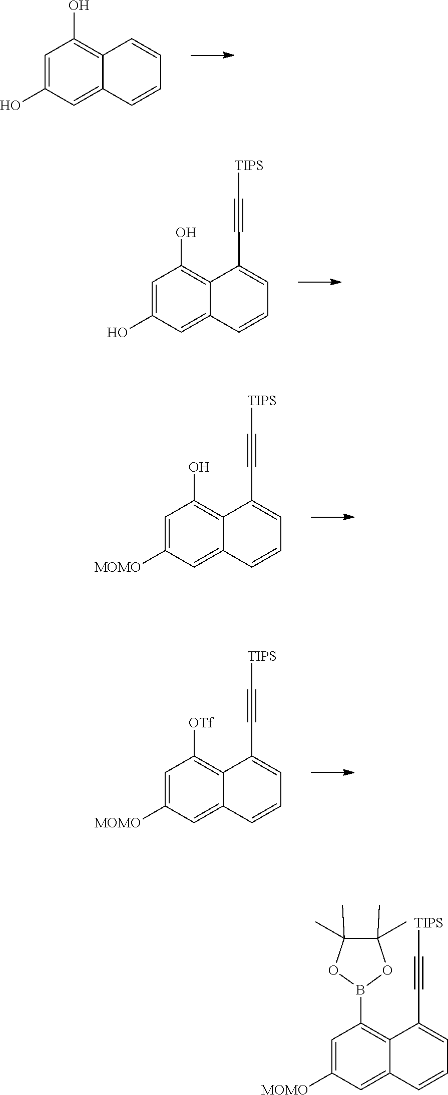

### Step 1: Synthesis of 8-(triisopropylsilyl) ethyne) naphthalene-1,3-diol

The compound naphthalene-1,3-diol (10.0 g, 62.43 mmol), 2-bromoacetynyl (triisopropyl) silane (19.58 g, 74.92 mmol), dichloro (p-methylisopropyl benzene) ruthenium (II) dimer (3.82 g, 6.24 mmol), and potassium acetate (12.26 g, 124.86 mmol) were dissolved in 1,4-dioxane (120 mL), and stir for 12 hours under nitrogen atmosphere at 110° C. After the reaction is completed, the reaction was cooled to room temperature, diluted the reaction solution with ethyl acetate, filtered, washed the organic phase with water, dried with anhydrous sodium sulfate, concentrated, and separated by column chromatography (PE/EA=50/1) to obtain a white like solid. (14.0 g, yield: 66%). ¹H NMR (400 MHz, CDCl₃) δ 9.30 (s, 1H), 7.62 (d, J=8.3 Hz, 1H), 7.46 (dd, J=7.1, 1.1 Hz, 1H), 7.29 (dd, J=8.2, 7.3 Hz, 1H), 6.74 (d, J=2.5 Hz, 1H), 6.62 (d, J=2.5 Hz, 1H), 4.91 (s, 1H), 1.25-1.14 (m, 21H).

### Step 2: Synthesis of 3-(methoxymethoxy)-8-(triisopropylsilyl) ethynyl) naphthalene-1-ol

The compound 8-((triisopropylsilyl) ethynyl) naphthalene-1 (14 g, 41.11 mmol) was dissolved in dichloromethane (100 mL). DIEA (16.99 mL, 102.78 mmol) and bromomethyl ether (4.03 mL, 49.33 mmol) were added under ice water bath cooling and stirred for 10 minutes. After the reaction was completed, the reaction solution was diluted with water, extracted with dichloromethane, the organic p by coluhase was dried with anhydrous sodium sulfate, concentrated, and separated by column chromatography (PE/EA=100/1) to obtain a yellowish oil. (13.6 g, yield: 86%). ¹H NMR (400 MHz, CDCl₃) δ 9.25 (s, 1H), 7.68 (d, J=8.2 Hz, 1H), 7.51-7.45 (m, 1H), 7.33-7.28 (m, 1H), 6.97 (d, J=2.4 Hz, 1H), 6.76 (d, J=2.4 Hz, 1H), 5.26 (s, 2H), 3.50 (s, 3H), 1.20-1.12 (m, 21H).

### Step 3: Synthesis of 3-(methoxymethoxy)-8-((triisopropylsilyl) ethynyl) naphthalen-1-yltrifluoromethanesulfonate

The compound 3-(methoxymethoxy)-8-((triisopropylsilyl) ethynyl) naphthalene-1-ol (13 g, 33.80 mmol) was dissolved in dichloromethane (100 mL), after the reaction was cooled to −40° C., DIEA (13.11 g, 101.41 mmol) was added, then dripped with trifluoromethanesulfonic anhydride (14.30 g, 50.70 mmol), and then stirred for 30 minutes. After the reaction was completed, the reaction was quenched with water, extracted with dichloromethane, concentrated, and separated by column chromatography (PE/EA=20/1) to obtain yellow oil. (16.23 g, yield: 93%). ¹H NMR (400 MHz, CDCl₃) δ 7.74 (dd, J=10.5, 4.3 Hz, 2H), 7.46-7.40 (m, 2H), 7.31 (d, J=2.3 Hz, 1H), 5.29 (s, 2H), 3.52 (s, 3H), 1.21-1.11 (m, 21H).

### Step 4: synthesis of triisopropyl (6-(methoxymethoxy)-8-(4-methoxymethoxy)-5-tetramethyl-1-methylidene-3-dioxoboropentane-2-yl) naphthalene-1-ethynyl) silane

The compound 3-(methoxymethoxy)-8-((triisopropylsilyl) ethynyl) naphthalen-1-yltrifluoromethanesulfonate (16.0 g, 30.97 mmol), 4,4,4′,4′,5,5,5′,5′-octamethyl-2,2′-bi(1,3,2-dioxaborolane) (15.73 g, 61.94 mmol), potassium acetate (10.64 g, 108.40 mmol) and Pd(dppf)Cl₂(2.26 g, 3.09 mmol) was dissolved in toluene (160 mL), after displacement with nitrogen, a stirred reaction was carried out in a nitrogen atmosphere at 110° C. for 12 h. After the reaction was completed, cooled to room temperature, add ethyl acetate to dilute the reaction solution, filter to remove insoluble substances, wash the organic phase with water, dry with anhydrous sodium sulfate, concentrate, and separate by column chromatography to obtain a light yellow solid. (6.2 g, yield: 41%). 1H NMR (400 MHz, CDCl3) δ 7.70-7.67 (m, 2H), 7.46 (d, J=2.1 Hz, 1H), 7.37-7.32 (m, 2H), 5.28 (s, 2H), 3.50 (s, 3H), 1.43 (s, 12H), 1.15 (s, 21H).

### Synthesis of Intermediate (1-(pyrrolidine-1-ylmethyl)cyclopropyl)methanol

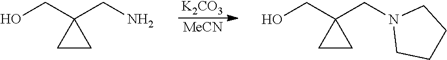

The compound (1-(aminomethyl) cyclopropyl) methanol (500 mg, 4.94 mmol) and 1,4-dibromobutane (1.12 g, 5.19 mmol) was dissolved in acetonitrile (20 mL), then potassium carbonate (1.78 g, 12.85 mmol) was added and stirred at room temperature for 12 hours. After the reaction was completed, filter off the solid, concentrate the organic phase and separate it by column chromatography (DCM/7M NH₃ in MeOH=100/1) to obtain a colorless oil. (390 mg, yield: 50.8%). ¹H NMR (400 MHz, CDCl₃) δ 3.55 (s, 2H), 2.70-2.53 (m, 6H), 1.83-1.69 (m, 4H), 0.49 (q, J=4.6 Hz, 2H), 0.36 (t, J=5.2 Hz, 2H).

### Synthesis of Intermediate 2-(7,8-difluoro-3-(methoxymethoxy)naphthalen-1-yl)-4,4,5,5-tetramethyl-1,3,2-dioxaborolane

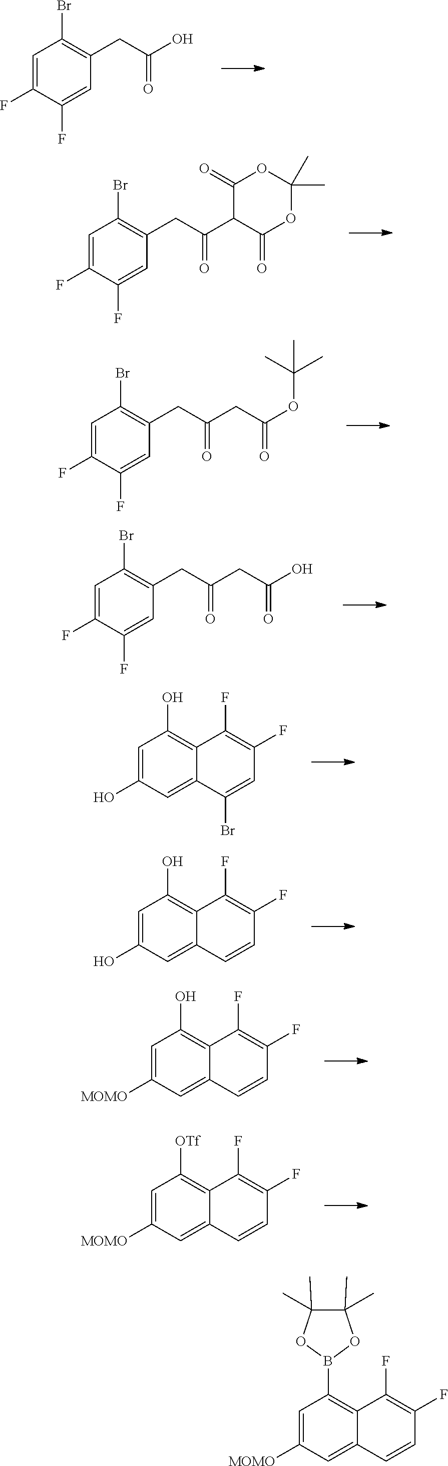

### Step 1: Synthesis of 5-(2-(2-bromo-4,5-difluorophenyl)acetyl)-2,2-dimethyl-1,3-dioxane-4,6-dione

Compound 2-(2-bromo-4) 5-difluorophenyl) acetic acid (1.5 g, 5.98 mmol) and Meldrum's acid (947 mg, 6.57 mmol) were dissolved in acetonitrile (20 mL), DMAP (73 mg, 0.60 mmol) and DIEA (1.66 g, 12.86 mmol) were added, then pivaloyl chloride (792 mg, 6.57 mmol) was slowly dropped into the reaction solution and stirred at room temperature for 3 hours. After the reaction was finished, 1N hydrochloric acid (35 mL) was added under ice bath cooling, the solid was precipitated, filtered, the solid was washed with water, and the white solid is obtained by vacuum drying. (2.2 g, yield: 97%). ¹H NMR (400 MHz, CDCl₃) δ 15.51 (s, 1H), 7.44 (dd, J=9.5, 7.4 Hz, 1H), 7.15 (dd, J=10.5, 8.0 Hz, 1H), 4.56 (s, 2H), 1.78 (s, 6H).

### Step 2: Synthesis of tert-butyl 4-(2-bromo-4,5-difluorophenyl)-3-oxobutanoate

Compound of 5-(2-(2-bromo-4,5-difluorophenyl)acetyl)-2,2-dimethyl-1,3-dioxane-4,6-dione (2.2 g, 5.83 mmol) was dissolved in tert-butanol (20 mL) and heated to 90° C. reaction for 16 hours. After the reaction was finished, concentrated reaction solution removes the tert-butyl alcohol to give an oil which was directly used for the next step. (2.0 g, yield: 100%). ¹H NMR (400 MHz, CDCl₃) δ 7.41 (dd, J=9.5, 7.5 Hz, 1H), 7.09 (dd, J=10.6, 8.1 Hz, 1H), 3.96 (s, 2H), 3.46 (s, 2H), 1.48 (s, 9H).

### Step 3: Synthesis of 4-(2-bromo-4,5-difluorophenyl)-3-oxobutanoic acid

Compound tert-butyl 4-(2-bromo-4,5-difluorophenyl)-3-oxobutanoate 2.0 g, 5.73 mmol) was dissolved in dichloromethane (20 mL), added trifluoroacetic acid (10 mL), and stirred at room temperature for 1 hour. After the reaction was completed, concentrate the reaction solution to obtain an off-white solid. (1.6 g, yield: 100%).

### Step 4: Synthesis of 5-bromo-7,8-difluoronaphthalene-1,3-diol

compound 4-(2-bromo-4,5-difluorophenyl)-3-oxobutanoic acid (1.6 g, 5.73 mmol) was dissolved in trifluoromethanesulfonic acid (20 mL), heated to 100° C., and stirred for 24 hours. After the reaction was completed, cooled to room temperature, slowly add the reaction solution to ice water, precipitate solids, filter to obtain crude products, and use petroleum ether to pulp and filter to obtain an off-white solid. (1.2 g, yield: 76%). ¹H NMR (400 MHz, DMSO) δ 10.53 (d, J=2.2 Hz, 1H), 10.14 (s, 1H), 7.94 (dd, J=10.0, 7.0 Hz, 1H), 6.90 (s, 1H), 6.66 (d, J=1.7 Hz, 1H).

### Step 5: Synthesis of 7,8-difluoronaphthalene-1,3-diol

Compound 5-bromo-7,8-difluoronaphthalene-1,3-diol (1.2 g, 4.36 mmol) was dissolved in methanol (20 mL), 10% Pd/C (0.2 g) was added in nitrogen atmosphere, hydrogen was replaced, and the reaction was stirred at room temperature in a hydrogen atmosphere for 2 hours. After the reaction was completed, the Pd/C was removed by filtration and washed with methanol. The organic phase was concentrated to give an off-white solid. (800 mg, yield: 93%).

### Step 6: Synthesis of 7,8-difluoro-3-(methoxymethoxy)naphthalen-1-ol

Compound 7,8-difluoronaphthalene-1,3-diol (0.80 g, 4.08 mmol) was dissolved in dichloromethane (10 mL). DIEA (791 mg, 6.12 mmol) and bromomethyl ether (535 mg, 6.12 mmol) were added under ice water bath cooling and stirred for 10 minutes. After the reaction was completed, the reaction solution was diluted with water, extracted with dichloromethane, the organic phase was dried with anhydrous sodium sulfate, concentrated, and separated by column chromatography (PE/EA=100/1) to give a yellowish oil. (0.9 g, yield: 92%).

### Step 7: Synthesis of 7,8-difluoro-3-(methoxymethoxy)naphthalen-1-yl trifluoromethanesulfonate

The compound 3-(methoxymethoxy)-8-((triisopropylsilyl) ethynyl) naphthalene-1-ol (0.9 g, 3.75 mmol) was dissolved in dichloromethane (10 mL), cooled to −40° C., added DIEA (1.45 g, 11.24 mmol), then dripped with trifluoromethanesulfonic anhydride (1.59 g, 5.62 mmol), and then stirred for 30 minutes. After the reaction was completed, the reaction was quenched with water, extracted with dichloromethane, concentrated, and separated by column chromatography (PE/EA=20/1) to obtain a white solid. (1.25 g, yield: 90%). 1H NMR (400 MHz, CDCl3) δ 7.58-7.52 (m, 2H), 7.46-7.44 (m, 1H), 7.43-7.36 (m, 1H), 7.29 (d, J=2.0 Hz, 1H), 5.30 (s, 2H), 3.54 (s, 3H).

### Step 8: Synthesis of 2-(7,8-difluoro-3-(methoxymethoxy)naphthalen-1-yl)-4,4,5,5-tetramethyl-1,3,2-dioxaborolane

Compound 7,8-difluoro-3-(methoxymethoxy)naphthalen-1-yl trifluoromethanesulfonate (1.25 g, 3.36 mmol), 4,4,4′,4′,5,5,5′,5′-octamethyl-2,2′-bi(1,3,2-dioxaborolane) (1.71 g, 6.72 mmol), potassium acetate (1.65 g, 16.80 mmol), and Pd(dppf)Cl₂ (249 mg, 0.34 mmol) were dissolved in DMF (10 mL), replaced with nitrogen, and heated to 80° C. for 12 hours in a nitrogen atmosphere. After the reaction was completed, cooled to room temperature, added ethyl acetate to dilute the reaction solution, filtered to remove insoluble substances, washed the organic phase with water, dried with anhydrous sodium sulfate, concentrated, and separated by column chromatography to obtain a light yellow solid. (0.9 g, yield: 76%).

¹H NMR (400 MHz, CDCl₃) δ 7.49-7.44 (m, 1H), 7.44-7.41 (m, 1H), 7.41-7.38 (m, 1H), 5.29 (s, 2H), 3.51 (s, 3H), 1.45 (s, 12H).

### Synthesis of Intermediate ((2-fluoro-6-(methoxymethoxy)-8-(4,4,5,5-tetramethyl-1,3,2-dioxaborolan-2-yl)naphthalen-1-yl)ethynyl)triisopropylsilane

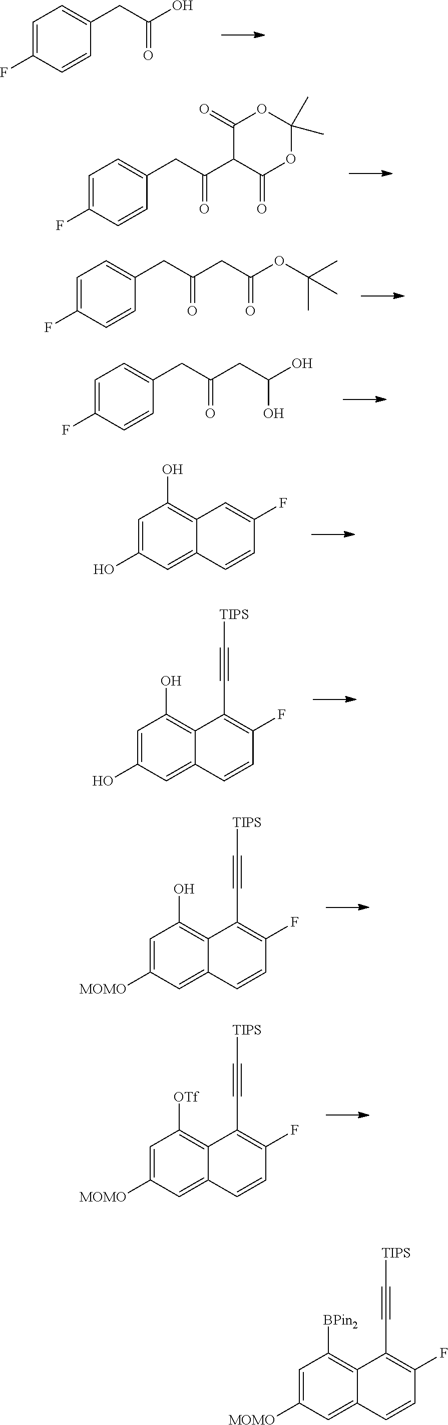

### Step 1: Synthesis of 5-(2-(4-fluorophenyl)acetyl)-2,2-dimethyl-1,3-dioxane-4,6-dione

2-(4-fluorophenyl) acetic acid (20 g, 130 mmol), Meldrum's acid (20.6 g, 143 mmol), and DMAP (1.58 g, 13 mmol) were dissolved in acetonitrile (100 mL), control temperature below 45° C., added DIPEA (36 g, 280 mmol), then added pivaloyl chloride (17.2 g, 143 mmol), stirred at room temperature for 4 hours, TLC (petroleum ether/ethyl acetate=1 bank 1) detection reaction was complete, drop 2N hydrochloric acid (100 mL) in ice bath, precipitate a large amount of yellow solid, filtrated, washed with water and dried to give a white solid. (32 g, yield: 88.1%). ¹H NMR (400 MHz, DMSO-d₆) δ 7.45-7.32 (m, 2H), 7.16 (t, J=8.9 Hz, 2H), 4.36 (s, 2H), 1.70 (s, 6H).

### Step 2: Synthesis of tert-butyl 4-(4-fluorophenyl)-3-oxobutanoate

5-(2-(4-fluorophenyl)acetyl)-2,2-dimethyl-1,3-dioxane-4,6-dione (32 g, 114 mmol) was dissolved in tert-butanol (300 mL) and heated to 90° C. reaction for 2 hours. The reaction was detected by TLC (petroleum ether/ethyl acetate=2-hand 1) completely, concentrated to dry, and directly used in the next reaction. ¹H NMR (400 MHz, DMSO-d₆) δ 7.26-7.08 (m, 4H), 3.86 (s, 2H), 3.54 (s, 2H), 1.40 (s, 9H).

### Step 3: Synthesis of 4-(4-fluorophenyl)-3-oxobutanoic acid

tert-butyl 4-(4-fluorophenyl)-3-oxobutanoate (29 g, 114 mmol) was dissolved in dichloromethane (200 mL). Trifluoroacetic acid (150 mL) was added and reacted at room temperature for 1 hour. The reaction was detected by TLC (petroleum ether/ethyl acetate=2 Acetate) completely, concentrated to dry and directly used in the next reaction. ¹H NMR (400 MHz, CDCl₃) δ 11.78 (s, 1H), 7.18 (dd, J=8.6, 5.5 Hz, 2H), 7.08-7.02 (m, 2H), 3.83 (s, 2H), 3.54 (s, 2H).

### Step 4: Synthesis of 7-fluoronaphthalene-1,3-diol

4-(4-fluorophenyl)-3-oxobutanoic acid (22.4 g, 114 mmol) was dissolved in trifluoromethanesulfonic acid (150 mL) and reacted at room temperature for 17 hours. TLC (petroleum ether/ethyl acetate=2/1) detected that the reaction was complete, dripping into ice water, ethyl acetate extraction, concentrated to a small amount of solvent, adding petroleum ether (500 mL), precipitation of a large number of solids, filtration, drying to a yellowish solid. (19.2 g, yield: 92.3%). ¹H NMR (400 MHz, DMSO-d₆) δ 10.18 (s, 1H), 9.48 (s, 1H), 7.63 (dd, J=9.0, 5.6 Hz, 1H), 7.56 (dd, J=10.8, 2.8 Hz, 1H), 7.24 (td, J=8.8, 2.8 Hz, 1H), 6.63 (d, J=2.1 Hz, 1H), 6.55 (d, J=2.1 Hz, 1H).

### Step 5: Synthesis of 7-fluoro-8-((triisopropylsilyl)ethynyl)naphthalene-1,3-diol

7-fluoronaphthalene-1,3-diol (5 g, 27.5 mmol), (bromoacetynyl) triisopropylsilane (7.53 g, 28.8 mmol), and potassium acetate (5.39 g, 55 mmol) were dissolved in dioxane (100 mL). Ruthenium dichloride and 1-isopropyl-4-toluene dimer (1.68 g, 2.75 mmol) were added, protected by argon gas, heated to 110° C. for 20 hours, and the reaction was completely detected by TLC (petroleum ether/ethyl acetate=2/1). The reaction was cooled to room temperature, extracted with ethyl acetate, concentrated, and purified by column chromatography to obtain a black oily substance. (7.4 g, yield: 75.2%).

### Step 6: Synthesis of 7-fluoro-3-(methoxymethoxy)-8-((triisopropylsilyl)ethynyl)naphthalen-1-ol

7-fluoro-8-((triisopropylsilyl)ethynyl)naphthalene-1,3-diol (7.4 g, 20.6 mmol) was dissolved in dichloromethane (100 mL), DIPEA (7.99 g, 61.9 mmol) and MOMBr (2.58 g, 20.6 mmol) were added to the mixture in ice bath, reacted at room temperature for 15 minutes, TLC (petroleum ether/ethyl acetate=5 ppg) was detected completely, water was added, extracted with dichloromethane, concentrated, and purified by column chromatography to obtain a yellow solid. (4.9 g, yield: 59%). ¹H NMR (400 MHz, CDCl₃) δ 9.12 (s, 1H), 7.65 (dd, J=9.1, 5.6 Hz, 1H), 7.18 (t, J=8.8 Hz, 1H), 6.96 (d, J=2.4 Hz, 1H), 6.80 (d, J=2.4 Hz, 1H), 5.24 (s, 2H), 3.50 (s, 3H), 1.18 (d, J=5.3 Hz, 21H).

### Step 7: Synthesis of 7-fluoro-3-(methoxymethoxy)-8-((triisopropylsilyl)ethynyl)naphthalen-1-yl trifluoromethanesulfonate

7-fluoro-3-(methoxymethoxy)-8-((triisopropylsilyl)ethynyl)naphthalen-1-ol (4.9 g, 12.2 mmol) was dissolved in dichloromethane (100 mL), added DIPEA (4.7 g, 36.6 mmol), cooled to −60° C., added trifluoromethanesulfonic anhydride (5.15 g, 18.3 mmol), reacted for 1 hour, TLC (petroleum ether/ethyl acetate=10 g) was detected completely, iced water was added, extracted with dichloromethane, concentrated, and purified by column chromatography to obtain a yellow oil. (6.0 g, yield: 92.3%). ¹H NMR (400 MHz, CDCl₃) δ 7.70 (dd, J=9.1, 5.4 Hz, 1H), 7.42 (d, J=2.4 Hz, 1H), 7.37-7.28 (m, 2H), 5.27 (s, 2H), 3.51 (s, 3H), 1.17 (d, J=6.0 Hz, 21H).

### Step 8: Synthesis of ((2-fluoro-6-(methoxymethoxy)-8-(4,4,5,5-tetramethyl-1,3,2-dioxaborolan-2-yl)naphthalen-1-yl)ethynyl)triisopropylsilane

7-fluoro-3-(methoxymethoxy)-8-((triisopropylsilyl)ethynyl)naphthalen-1-yl trifluoromethanesulfonate (5.0 g, 9.35 mmol), diborate pinacol ester (4.75 g, 18.7 mmol), and potassium acetate (2.75 g, 28.1 mmol) were dissolved in toluene (200 mL). [1,1-bis (diphenylphosphorus) ferrocene]palladium dichloride (684 mg, 0.935 mmol) was added, protected by argon gas, and heated to 130° C. for 5 hours. TLC (petroleum ether/ethyl acetate=5° C.) detected that the reaction was complete, cooled to room temperature, filtered, concentrated to dry, added water, extracted with ethyl acetate, concentrated and purified by column chromatography. (2.4 g, yield: 50.1%). ¹H NMR (400 MHz, CDCl₃) δ 7.65 (dd, J=9.0, 5.6 Hz, 1H), 7.50 (d, J=2.6 Hz, 1H), 7.37 (d, J=2.6 Hz, 1H), 7.21 (t, J=8.8 Hz, 1H), 5.26 (s, 2H), 3.50 (s, 3H), 1.43 (s, 12H), 1.15 (d, J=2.4 Hz, 21H).

### Synthesis of the intermediate 2-(4-(benzyloxy)-1,1a, 6,6a-tetrahydrocyclopropa[a]inden-2-yl)-4,4,5,5-tetramethyl-1,3,2-dioxaborolane

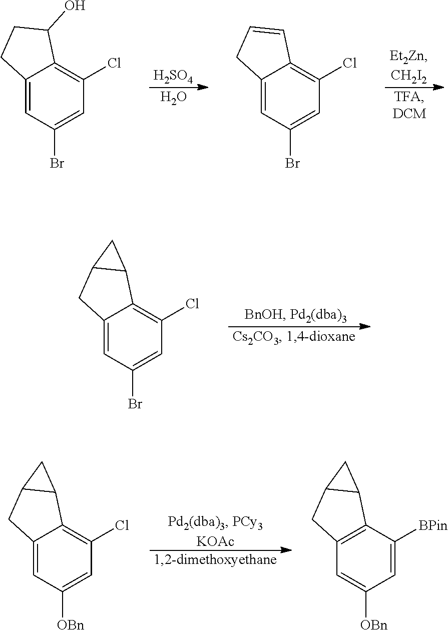

### Step 1: Synthesis of 6-bromo-4-chloro-1H-indene

Mix concentrated sulfuric acid (8 mL) with water (40 mL). Add 5-bromo-7-chloro-2,3-dihydro-1H-inden-1-ol (5 g, 20.2 mol). The reaction mixture was stirred under reflux for 12 hours. After cooled to room temperature, water (150 mL) and methyl tert-butyl ether (150 mL) were added. After shaking and delamination, the aqueous phase was extracted by methyl tert-butyl ether (50 mL). The combined organic phase was washed with saturated sodium bicarbonate solution (50 mL) and saturated salt solution (50 mL), dried with sodium sulfate and evaporated at reduced pressure. The residue was purified by column chromatography (silica gel, petroleum ether) to obtain a colorless oil (4.2 g, yield: 90%).

### Step 2: Synthesis of 4-bromo-2-chloro-1,1a, 6,6a-tetrahydrocyclopropa[a]indene

The anhydrous dichloromethane (20 mL) solution of diethyl zinc/n-hexane (2m mL 18.3 min) was cooled in an ice water bath. A solution of Trifluoroacetic acid (2.72 mL, 36.6 mmol) in dichloromethane (20 mL) was dripped slowly. After stirring at this temperature for 20 minutes, an anhydrous dichloromethane (20 mL) solution of diiodomethane (2.96 mL, 36.6 mmol) was added. After continuing stirring for 20 minutes, a solution of anhydrous dichloromethane (10 mL) of 6-bromo-4-chloro-1H-indene (4.2 g, 18.3 mmol) slowly added dropwise. Removed the ice bath and stirred for 24 hours. Slowly added dilute hydrochloric acid (1N mL 150 min) under stirring. The mixture was extracted by petroleum ether (200 mL×2). The combined organic phase was washed with brine, dried with sodium sulfate and evaporated at reduced pressure to give a yellow oil. purified by column chromatography (silica gel, petroleum ether) to obtain 4-bromo-2-chloro-1,1a, 6,6a-tetrahydrocyclopropa[a]indene as a colorless oil (3.79 g, yield 85%).

### Step 3: Synthesis of 4-(benzyloxy)-2-chloro-1,1a, 6,6a-tetrahydrocyclopropa[a]indene

Under nitrogen atmosphere, pour pre deoxidized 1,4-dioxane (100 mL) into a flask containing 4-bromo-2-chloro-1,1a, 6,6a-tetrahydrocyclopropane[a]indene (3.79 g, 15.6 mmol), tribenzylidene acetone dipalladium (2.75 g, 3.0 mmol), benzyl alcohol (2.05 g, 19 mmol), and cesium carbonate (7.86 g, 30 mmol). The reaction mixture was stirred at 100° C. for 15 hours. After the reaction cooled to room temperature, added methyl tert butyl ether (200 mL) and water (200 mL) for extraction. After separation, the organic phase was washed with brine (50 mL), and then dried with sodium sulfate, concentrated to obtain a viscous substance under reduced pressure. The product was obtained as a yellow solid (1.9 g, yield: 45%) through column chromatography (silica gel, ethyl acetate:petroleum ether=1:20).

### Step 4: Synthesis of 2-(4-(benzyloxy)-1,1a, 6,6a-tetrahydrocyclopropa[a]inden-2-yl)-4,4,5,5-tetramethyl-1,3,2-dioxaborolane

Under the protection of argon gas, a mixture of 4-(benzyloxy)-2-chloro-1,1a, 6,6a-tetrahydrocyclopropa[a]indene (1.9 g, 7.0 mmol), bipinnacol borate (3.05 g, 12 mmol), tribenzylidene acetone dipalladium (1.3 g, 1.4 mmol), tricyclohexylphosphine (0.785 g, 2.8 mmol), potassium acetate (1.39 g, 14 mmol), and ethylene glycol dimethyl ether (25 mL) were heated in microwave for 2 hours at 150° C. After cooling the reaction to room temperature, add ethyl acetate (100 mL) and water (100 mL) for extraction. The aqueous phase was extracted with ethyl acetate (50 mL). The combined organic phase was washed with brine (30 mL), dried with sodium sulfate, and subjected to reduced pressure rotary evaporation to obtain a viscous substance. A yellow solid product (1.72 g, yield 68%) was obtained by column chromatography (silica gel, ethyl acetate:petroleum ether=1:20).

### Synthesis of Intermediate tert-butyl (1S,5R)-2-oxo-3,8-diazabicyclo[3.2.1]octane-8-carboxylate

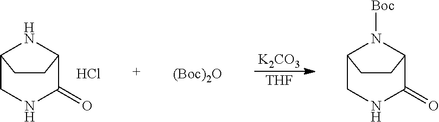 [SMILES: "(Boc)2O" ; O=C1NCC2CCC1N2 ; Cl ; "K2CO3" ; CC(C)(C)OC(=O)N1C2CCC1C(=O)NC2 ; "THF"]

### Step 1: Synthesis of tert-butyl (1S,5R)-2-oxo-3,8-diazabicyclo[3.2.1]octane-8-carboxylate

3,8-diazabicyclo [3.2.1] oct-2-one hydrochloride (97 mg, 0.6 mmol) and potassium carbonate (165 mg, 1.2 mmol) were dissolved in tetrahydrofuran (2 mL), di-tert-butyl dicarbonate (157 mg, 0.72 mmol) was added. The mixture was stirred overnight. After the reaction was completed, the solid was filtered off, and the filtrate is concentrated before column chromatography to obtain a white solid. (116 mg, yield: 85%). MS m/z: 227.33 [M+H]+.

### Synthesis of intermediate 2-(8-methoxy-3-(methoxymethoxy)-5,6,7,8-tetrahydronaphthalen-1-yl)-4,4,5,5-tetramethyl-1,3,2-dioxaborolane

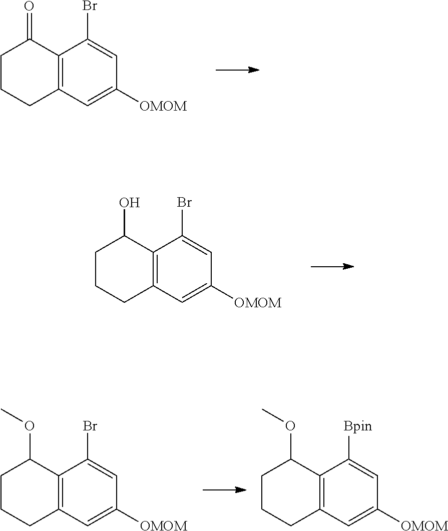

### Step 1: Synthesis of 8-bromo-6-(methoxymethoxy)-1,2,3,4-tetrahydronaphthalen-1-ol

Under argon atmosphere, 8-bromo-6-methoxymethoxy-3,4-dihydronaphthalen-1-one (146 mg, 0.5 mmol) was dissolved in methanol (2 mL), and sodium borohydride (38 mg, 1 mmol) was added at 0° C. Slowly increase the temperature to room temperature and stirred overnight. After the reaction was completed, concentrated the reaction solution under reduced pressure. Saturated ammonium chloride solution (2 mL) was added to the bottle and extracted with cyclohexane (2 mL×3). The extracted organic phase was washed with saturated salt water, dried with sodium sulfate and concentrated under reduced pressure to obtain a colorless oily product (about 150 mg). No further purification is required.

### Step 2: Synthesis of 8-bromo-1-methoxy-6-(methoxymethoxy)-1,2,3,4-tetrahydronaphthalene

Under argon atmosphere, 8-bromo-6-methoxymethoxy-1,2,3,4-tetrahydronaphthalen-1-ol (150 mg, 0.5 mmol) was dissolved in dry DMF, and sodium hydride (320 mg, 4 mmol, 60% mixed in mineral oil) and iodomethane (0.62 mL, 5 mmol) were added. The suspension was stirred at room temperature for 2 hours. After the complete conversion, concentrated the reaction solution under reduced pressure and pulped with petroleum ether, and filtered, and the crude product was separated by silica gel column chromatography (petroleum ether:ethyl acetate=20:1) to obtain a colorless oil 100 mg. ¹H NMR (400 MHz, CDCl₃) δ 7.16 (d, J=2.5 Hz, 1H), 6.73 (d, J=2.5 Hz, 1H), 5.12 (s, 2H), 4.39 (t, J=2.8 Hz, 1H), 3.46 (d, J=15.2 Hz, 7H), 2.92-2.73 (m, 1H), 2.65 (ddd, J=17.2, 12.1, 5.8 Hz, 1H), 2.38-2.25 (m, 1H). 1.90 (tdq, J=13.4, 8.3, 2.8 Hz, 1H), 1.70 (dh, J=13.0, 3.1 Hz, 1H), 1.49 (tt, J=13.8, 3.3 Hz, 1H).

### Step 3: Synthesis of Intermediate 2-(8-methoxy-3-(methoxymethoxy)-5,6,7,8-tetrahydronaphthalen-1-yl)-4,4,5,5-tetramethyl-1,3,2-dioxaborolane

In argon atmosphere, 8-bromo-1-methoxy-6-methoxymethoxy-1 mg (98 mg, 0.33 mmol) was dissolved in tetrahydrofuran (2 mL), and the temperature was reduced to low temperature by using dry ice ethanol bath. After that, n-butyl lithium (0.2 mL, 2.5m in THF) was added to the solution. Keep low temperature, stir for 40 minutes, 2-isopropoxy-4,4,5,5-tetramethyl-1,3,2-dioxaborolane (89 mg, 0.48 mmol) was add. Then slowly warm up to room temperature. After the reaction was completed, concentrated the reaction solution under reduced pressure and pulped with petroleum ether, purified by TLC to give a colorless oil 39 mg. ¹H NMR (400 MHz, CDCl₃) δ 7.24 (d, J=2.8 Hz, 1H), 6.81 (d, J=2.8 Hz, 1H), 5.28-5.07 (m, 2H), 4.84 (t, J=4.0 Hz, 1H), 3.52-3.35 (m, 6H), 2.87-2.58 (m, 2H), 2.15 (d, J=13.9 Hz, 1H), 1.91 (d, J=13.0 Hz, 1H). 1.78-1.61 (m, 2H), 1.43-1.26 (m, 12H).

The preparation method of intermediate 2-(8-methoxy-3-methoxymethoxy)-5,6,7,8-tetrahydronaphthalen-1-yl-4,4,5,5,5-tetramethyl-1,3,2-dioxabiorane was used to obtain 2-(3-methoxy-6-(methoxymethoxy)-2,3-dihydro-1H-inden-4-yl)-4,4,5,5-tetramethyl-1,3,2-dioxaborolane m/z: 334.72 [M+H]⁺.

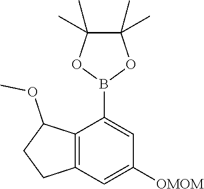

Synthesis of intermediate 2-(5-(methoxymethoxy)-7b-methyl-1a, 2,3,7b-tetrahydro-1H-cyclopropa[a]naphthalen-7-yl)-4,4,5,5-tetramethyl-1,3,2-dioxaborolane:

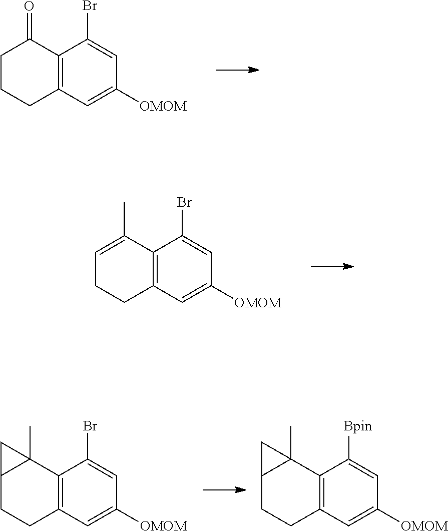

### Step 1: Synthesis of 5-bromo-7-(methoxymethoxy)-4-methyl-1,2-dihydronaphthalene

MePPh3I (1.78 g, 5 mmol) was dissolved in 40 ml anhydrous tert-butyl methyl ether and t-BuOK/THF (5 ml, 5 mmol) was added in argon atmosphere, the reaction system turns orange. After the reaction system was stirred for 0.5 hour, 8-bromo-6-methoxy-3-dihydronaphthalene-1-one (284 mg, 1 mmol) was added, and the reaction system was stirred overnight at room temperature. After the reaction was completed, the reaction was quenched with 20 ml water, extracted with 100 ml ethyl acetate, the organic phase was dried by anhydrous sodium sulfate, the crude product was decompressed and removed from the solvent, and the crude product was separated by column chromatography (EA:PET/1:10) to obtain a light yellow liquid (221 mg, yield: 78%). 1H NMR (400 MHz, CDCl3) δ 7.08 (d, J=2.6 Hz, 1H), 6.79-6.69 (m, 1H), 5.91 (td, J=5.2, 1.5 Hz, 1H), 5.07 (s, 2H), 3.40 (s, 3H), 2.55 (dd, J=8.7, 6.2 Hz, 2H), 2.25 (Q, J=1.6 Hz, 3H), 1.98 (dtt, J=9.1, 5.4). 1.7 Hz, 2H).

### Step 2: Synthesis of 7-bromo-5-(methoxymethoxy)-7b-methyl-1a, 2,3,7b-tetrahydro-1H-cyclopropa[a]naphthalene

Under argon atmosphere, a diethyl zinc solution (0.23 mL, 2M in n-hexane) was dissolved in 3 mL of dichloromethane, and trifluoroacetic acid (34 mg, 0.3 mmol) was added. The mixture was stirred at 0° C. for 20 minutes, followed by the addition of diiodomethane (120 mg, 0.45 mmol) to the reaction. After 20 minutes, 5-bromo-7-methoxymethoxy-4-methyl-1,2-dihydronaphthalene (42 mg, 0.15 mmol) was added to the reaction. Slowly raise the temperature to room temperature and stir overnight. After the reaction was completed, the reaction was quenched with saturated ammonium chloride solution (4 mL), extracted with ethyl acetate (3 mL×3). The organic phase was washed with brine (3 mL), then dried with sodium sulfate and concentrated under reduced pressure to give a residue which was separated by pre-TLC to give the product (20 mg, yield: 44.8%).

### Step 3: Synthesis of 2-(5-(methoxymethoxy)-7b-methyl-1a, 2,3,7b-tetrahydro-1H-cyclopropa[a]naphthalen-7-yl)-4,4,5,5-tetramethyl-1,3,2-dioxaborolane

Compound 7-bromo-5-methoxymethoxy-7b-methyl-1a, B₂Pin₂ (25 mg, 0.067 mmol), KOAc (16 mg, 0.1). 16 mmol) and Pd (dppf) Cl₂ (11 mg, 0.013 mmol) were dissolved in 1 ml DMF, and the reaction system was heated to 110° C. and stirred overnight under argon. After the reaction was completed, the crude product of the solvent is removed under reduced pressure, and the crude product is separated by column chromatography to obtain a colorless oil (4 mg, yield: 17%).

### Synthesis of Intermediate 1-ethynyl-3-(methoxymethoxy) naphthalene

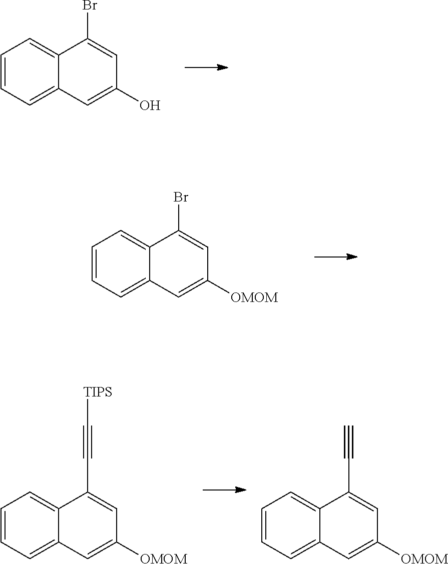

### Step 1: Synthesis of 1-bromo-3-(methoxymethoxy) naphthalene

4-bromonaphthalene-2-phenol (1.0 g, 4.48 mmol) and DIEA (1.49 mL, 8.96 mmol) were dissolved in 20 ml DCM, and MOMBr (0.44 mL, 5.4 mmol) was slowly added under ice water bath. After the addition is completed, the system turns pale yellow, and the reaction system continues to be stirred for 5 minutes. After the TLC monitoring reaction is completed, add 10 ml of water to the reaction system for quenching, and then add 50 mL DCM for extraction and separation. The organic phase was dried with anhydrous sodium sulfate and the crude product of the solvent was removed under reduced pressure. The crude product was separated by column chromatography (eluent: EA:PET/1:10) to obtain a colorless oily liquid (1.1 g, yield: 91.7%). MS m/z: 267 [M+H]⁺. ¹H NMR (400 MHz, CDCl₃) δ 8.10-8.05 (m, 2H), 7.77 (s, 1H), 7.50-7.40 (m, 2H), 7.30 (s, 1H), 6.12 (s, 2H), 3.30 (s, 3H).

### Step 2: Synthesis of triisopropyl ((3-(methoxymethoxy) naphthalen-1-yl) ethynyl) silane

The compound 1-bromo-3-(methoxymethoxy) naphthalene (534 mg, 2 mmol) and diethylamine (2 mL) was dissolved in 2 mL THF. After displacing argon gas, CuI and Pd(PPh₃)₄ was added. After displacing argon gas again, 1 mL of triisopropylsilyne was added. the reaction mixture was heated to 65° C. and stir overnight. After the reaction was completed, the organic solvent was removed under reduce pressure to obtain a crude product. The crude product was separated by column chromatography (eluent: EA:PET/1:10) to obtain a colorless oily liquid (420 mg, yield: 57%). MS m/z: 369 [M+H]⁺. ¹H NMR (400 MHz, CDCl₃) δ 8.31-8.15 (m, 1H), 7.97-7.65 (m, 1H), 7.50-7.40 (m, 2H), 7.30 (s, 1H), 6.12 (s, 2H), 3.30 (s, 3H), 1.50-1.40 (m, 3H), 0.97 (d, J=4.2 Hz, 18H).

### Step 3: Synthesis of 1-ethynyl-3-(methoxymethoxy) naphthalene

The compound triisopropyl ((3-(methoxymethoxy) naphthalene-1-yl) ethynyl) silane (420 mg, 1.14 mmol) was dissolved in 3 ml DMF solvent, then CsF (866 mg, 5.70 mmol) was added and stirred at room temperature overnight under argon protection. After the reaction is completed, add 100 mL of EA and 10 mL of water for extraction. After separation, wash the organic phase twice with 10 mL of water, and wash three times with 100 mL of saturated sodium chloride solution, the organic phase was dried with anhydrous sodium sulfate and the crude product of the solvent was removed under reduced pressure. The crude product was purified by PLC (eluent: EA:PET/1:5) to obtain a light red oily liquid (200 mg, yield: 88%). MS m/z: 213 [M+H]⁺.

### Synthesis of Intermediate 2-(3-ethyl-6-(methoxymethoxy)-2,3-dihydro-1H-inden-4-yl)-4,4,5,5-tetramethyl-1,3,2-dioxaborolane

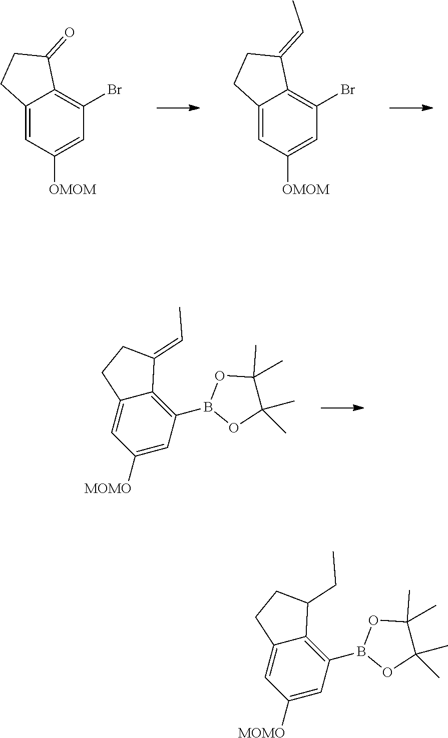

### Step 1: Synthesis of (E)-7-bromo-1-ethylidene-5-(methoxymethoxy)-2,3-dihydro-1H-indene

EtPPh₃I (2.09 g, 5 mmol) was dissolved in 40 mL anhydrous tert-butyl methyl ether and t-BuOK/THF (5 mL, 5 mmol) was added in argon atmosphere, the reaction system turns orange yellow. After the reaction system was stirred for 1 hour, the THF solution of 7-bromo-5-methoxymethoxy-2methoxy-3-dihydro-1H-indole-1-one (271 mg, 1 mmol) was added, and the reaction system was stirred overnight at room temperature. After the reaction was completed, the reaction system was quenched with 20 mL water, extracted with 100 mL ethyl acetate, the organic phase was dried by anhydrous sodium sulfate, and the crude product was decompressed to remove the solvent. The crude product was separated by column chromatography (EA:PET/1:10) to obtain a light yellow liquid (180 mg, yield: 64%). MS m/z: 283 [M+H]⁺. ¹H NMR (400 MHz, CDCl₃) δ 7.01 (dd, J=5.2, 2.2 Hz, 1H), 6.82 (dd, J=12.5, 2.2 Hz, 1H), 6.74-6.63 (m, 1H), 5.07 (d, J=5.9 Hz, 2H), 3.40 (d, J=4.2 Hz, 3H), 2.86 (dd, J=9.1, 5.6 Hz, 1H). 2.77-2.64 (m, 2H), 2.60 (t, J=6.9 Hz, 1H), 1.83 (dt, J=7.3, 1.6 Hz, 1H), 1.74 (dt, J=7.0, 1.6 Hz, 2H).

### Step 2: synthesis of (E)-2-(3-ethylidene-6-(methoxymethoxy)-2,3-dihydro-1H-inden-4-yl)-4,4,5,5-tetramethyl-1,3,2-dioxaborolane

Dissolve (E)-7-bromo-1-ethylidene-5-(methoxymethoxy)-2,3-dihydro-1H-indene (180 mg, 0.64 mmol), B₂Pin₂(324 mg, 1.28 mmol), KOAc (126 mg, 1.28 mmol) and Pd(dppf)Cl₂(47 mg, 0.064 mmol) in 10 mL of dioxane. Run evacuating and backfilling with argon and then heat the reaction at 100° C. overnight. After completion, remove the solvent under vacuum to give crude product. The crude product was purified with column chromatography to give colorless oil (130 mg, yield: 61%). MS m/z: 331[M+H]⁺ 0.1H NMR (400 MHz, CDCl3) δ 7.16 (d, J=2.5 Hz, 1H), 6.90 (d, J=2.5 Hz, 1H), 5.13-5.03 (m, 3H), 3.40 (s, 4H), 2.95-2.75 (m, 1H), 2.72-2.61 (m, 1H), 2.17-1.91 (m, 2H), 1.84 (ddt, J=12.7, 8.0, 1.5 Hz, 1H), 1.32-1.27 (m, 1H), 1.23-1.14 (m, 5H), 0.88 (t, J=7.3 Hz, 4H)

### Step 3: synthesis of 2-(3-ethyl-6-(methoxymethoxy)-2,3-dihydro-1H-inden-4-yl)-4,4,5,5-tetramethyl-1,3,2-dioxaborolane

Dissolve compound (E/S)-2-(3-ethylidene-6-(methoxymethoxy)-2,3-dihydro-1H-inden-4-yl)-4,4,5,5-tetramethyl-1,3,2-dioxaborolane (130 mg, 0.39 mmol) in 5 mL of ethyl acetate. Add 120 mg of Pd/C (10%) and then run evacuating and backfilling with hydrogen. The reaction was stirred at room temperature overnight. After completion, filter the reaction mixture. The solvent was removed under vacuum to leave pasty solid (100 mg, yield: 80%). MS m/z: 331[M+H]+. 1H NMR (400 MHz, CDCl3) δ 7.16 (d, J=2.5 Hz, 1H), 6.90 (d, J=2.5 Hz, 1H), 6.02 (s, 2H), 3.40 (s, 3H), 3.33-3.22 (m, 2H), 2.72-2.61 (m, 1H), 2.17-1.52 (m, 6H), 1.25 (s, 12H), 0.88 (t, J=7.3 Hz, 3H)

### Example 1: Synthesis of 4-(4-((1R,5S)-3,8-diazabicyclo[3.2.1]octan-3-yl)-8-fluoro-2-((1-(pyrrolidin-1-ylmethyl)cyclopropyl)methoxy)pyrido[4,3-d]pyrimidin-7-yl)-5-ethynyl naphthalen-2-ol

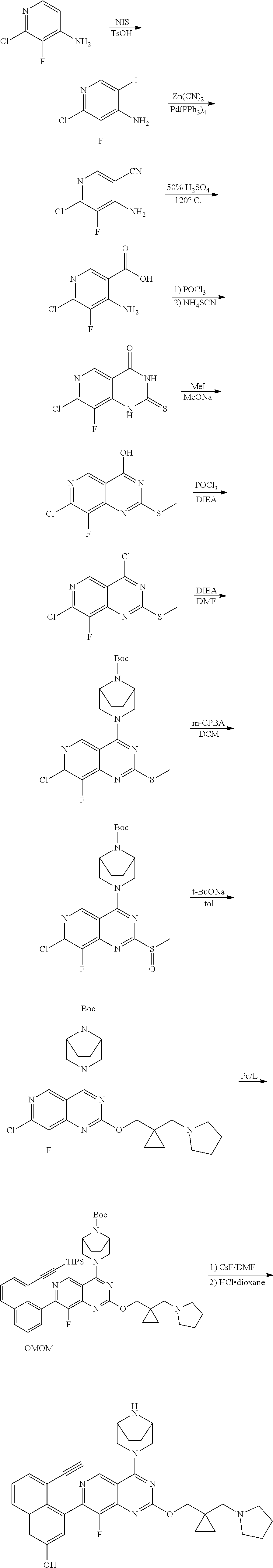

### Step 1: synthesis of 2-chloro-3-fluoro-5-iodopyridin-4-amine

2-chloro-3-fluoropyridin-4-amine (4.22 g, 28.80 mmol) was dissolved in acetonitrile (50 mL). NIS (7.77 g, 34.55 mmol) and p-toluene sulfonic acid (248 mg, 1.44 mmol) were added in. The reaction was heated at 70° C. for 16 hours. After completion, the reaction was cooled to room temperature. Water was added to dilute the reaction mixture. Solid precipitated. Solid was collected by filtering. Solid was eluted with saturated aqueous Na2S2O3 and water successively and then dried in vacuum to give the target compound which was used in next step directly. (7.5 g, yield: 98%). 1H NMR (400 MHz, CDCl3) δ 8.17 (s, 1H), 4.83 (s, 2H).

### Step 2: synthesis of 4-amino-6-chloro-5-fluoronicotinonitrile

Dissolve 2-chloro-3-fluoro-5-iodopyridin-4-amine (7.7 g, 28.26 mmol) and Zn(CN)2 (4.32 g, 36.74 mmol) in DMF (150 mL). Add Pd(PH3)4 (1.63 g, 1.41 mmol) and 4 Å molecular sieves (2.5 g). Run evacuating and backfilling with nitrogen. Heat and stir the reaction at 100° C. for 3 hours under nitrogen atmosphere. After completion, remove solid by filtering. Let the filtrate cooled to room temperature and add 300 mL of water to dilute the reaction solution. Solid precipitated. Filter the mixture and elute the solid by water. Solid was dried in vacuum to give the crude product which was used directly in next step. (4.85 g, yield: 100%). 1H NMR (400 MHz, DMSO) δ 8.20 (s, 1H), 7.66 (s, 2H).

### Step 3: synthesis of 4-amino-6-chloro-5-fluoronicotinic acid

Dissolve 4-amino-6-chloro-5-fluoronicotinonitrile (4.85 g, 28.26 mmol) in 50% H2SO4 (50 mL). Heat the mixture at 120° C. and stirred it for 6 hours. After completion, let the mixture cooled to room temperature. Poured the mixture into ice slowly. Solid precipitated. Filter and elute the solid with water. Dissolve the solid with ethyl acetate and wash the solution with saturated aqueous solidum carbonate. The aqueous layer was treated with 10% aqueous hydrochloric acid to pH 2-3. Solid precipitated. Collected the solid by filtering and dried the solid under vacuum to give off-white solid. (4.62 g, yield: 85.8%). 1H NMR (400 MHz, DMSO) δ 8.36 (s, 1H), 7.59 (s, 2H).

### Step 4: synthesis of 7-chloro-8-fluoro-2-thioxo-2,3-dihydropyrido[4,3-d]pyrimidin-4(1H)-one

A mixture of 4-amino-6-chloro-5-fluoronicotinic acid and POCl3 (50 mL) was heated at 90° C. and stirred for 4 hours. After completion, the mixture was cooled to room temperature and then concentrated. The resulting oil was dissolved in anhydrous THF (20 mL). The solution was added dropwise to a solution of ammonium thiocyanate (3.67 g, 48.28 mmol) in THF (80 mL). The mixture was stirred at room temperature for 24 hours. Added water and extract the mixture with ethyl acetate. The organic layer was washed with brine, dried over Na2SO4 and then concentrated to give yellow solid. 10 mL of ethyl acetate was added. Solid was crushed and collected by filtering to give light yellow solid. (4.52 g, yield: 80.8%). 1H NMR (400 MHz, DMSO) δ 13.29 (s, 1H), 12.85 (s, 1H), 8.64 (s, 1H).

### Step 5: synthesis of 7-chloro-8-fluoro-2-(methylthio)pyrido[4,3-d]pyrimidin-4-ol

Dissolve 7-chloro-8-fluoro-2-thioxo-2,3-dihydropyrido[4,3-d]pyrimidin-4(1H)-one (4.52 g, 19.51 mmol) in anhydrous DMF (50 mL). Add sodium methoxide (1.06 g, 19.51 mmol). After stirring at room temperature for 10 min, add methyl iodide (2.77 g, 1.21 mL, 19.51 mmol) and stir the reaction at room temperature for 2 hours. After completion, add cooled water. Solid precipitated. Collected the solid by filtering and elute the solid by water. Drying in vacuum gave yellow solid (3.0 g, yield: 66%). 1H NMR (400 MHz, DMSO) δ 13.24 (s, 1H), 8.81 (s, 1H), 2.62 (s, 3H).

### Step 6: synthesis of 4,7-dichloro-8-fluoro-2-(methylthio)pyrido[4,3-d]pyrimidine

Dissolve 7-chloro-8-fluoro-2-(methylthio)pyrido[4,3-d]pyrimidin-4-ol (420 mg, 1.71 mmol) in POCl3 (4 mL) and then add DIEA (442 mg, 3.42 mmol). Heat the reaction at 90° C. for 3 hours. After completion, cool the reaction to room temperature. Run concentration to remove excess POCl3. Dissolve the residue in ethyl acetate, wash the solution by water and brine successively. Dry the organic layer with sodium sulfate and the concentrate it to give crude product which was used directly in next step. (450 mg, yield: 100%).

### Step 7: synthesis of tert-butyl (1R,5S)-3-(7-chloro-8-fluoro-2-(methylthio)pyrido[4,3-d]pyrimidin-4-yl)-3,8-diazabicyclo[3.2.1]octane-8-carboxylate

Dissolve 4,7-dichloro-8-fluoro-2-(methylthio)pyrido[4,3-d]pyrimidine (120 mg, 0.46 mmol) and tert-butyl (1R,5S)-3,8-diazabicyclo[3.2.1]octane-8-carboxylate (107 mg, 0.50 mmol) in anhydrous DMF (5 mL). Add DIEA (89 mg, 0.69 mmol) with cooling by ice-water bath. The reaction was stirred in ice-water batch for 30 min. After completion, add water to dilute the reaction. Extract the reaction with ethyl acetate. The organic layer was washed with brine twice and then dried over sodium sulfate. Concentration gave yellow solid. Used petroleum ether to wash the solid and run filtering and drying under vacuum to give light yellow solid. (200 mg, yield: 99%). 1H NMR (400 MHz, CDCl3) δ 8.71 (s, 1H), 4.57-4.28 (m, 4H), 3.65 (s, 2H), 2.62 (s, 3H), 2.08-1.84 (m, 4H), 1.69 (d, J=7.4 Hz, 2H), 1.52 (s, 9H).

### Step 8: synthesis of tert-butyl (1R,5S)-3-(7-chloro-8-fluoro-2-(methylsulfinyl)pyrido[4,3-d]pyrimidin-4-yl)-3,8-diazabicyclo[3.2.1]octane-8-carboxylate

Dissolve tert-butyl (1R,5S)-3-(7-chloro-8-fluoro-2-(methylthio)pyrido[4,3-d]pyrimidin-4-yl)-3,8-diazabicyclo[3.2.1]octane-8-carboxylate (200 mg, 0.45 mmol) in DCM (10 mL). Cooled the solution in ice-water bath to 0° C.-10° C., then add 85% m-CPBA (116 mg, 0.54 mmol). The reaction was stirred at 0° C.-10° C. for 20 min. After completion, quench the reaction by adding saturated aqueous sodium thiosulfate. Use DCM to extract the reaction. The organic layer was washed with saturated aqueous sodium bicarbonate and brine successively and dried over sodium sulfate. Concentration gave light yellow solid which was used directly in next step. (200 mg, yield: 99%). 1H NMR (400 MHz, CDCl3) δ 8.91 (s, 1H), 4.77-4.35 (m, 4H), 3.77 (s, 2H), 2.98 (s, 3H), 2.08-1.84 (m, 4H), 1.69 (d, J=7.4 Hz, 2H), 1.52 (s, 9H).

### Step 9: synthesis of tert-butyl (1R,5S)-3-(7-chloro-8-fluoro-2-((1-(pyrrolidin-1-ylmethyl)Cyclopropyl)methoxy)pyrido[4,3-d]pyrimidin-4-yl)-3,8-diazabicyclo[3.2.1]octane-8-carboxylate

Dissolve tert-butyl (1R,5S)-3-(7-chloro-8-fluoro-2-(methylsulfinyl)pyrido[4,3-d]pyrimidin-4-yl)-3,8-diazabicyclo[3.2.1]octane-8-carboxylate (200 mg, 0.44 mol) and (1-(pyrrolidin-1-ylmethyl)cyclopropyl)methanol (82 mg, 0.53 mmol) in anhydrous toluene (5 mL). Add sodium tert-butoxide (55 mg, 0.57 mmol) under cooling with ice-water bath. Stir the reaction in ice-water bath for 30 min. After completion, quench the reaction by adding ice-water. Use ethyl acetate to extract the reaction. The organic layer was dried over sodium sulfate and then concentrated. Isolation with column chromatography gave off-white solid. (150 mg, yield: 62.5%)

### Step 10: synthesis of tert-butyl (1R,5S)-3-(8-fluoro-7-(3-(methoxymethoxy)-8-((triisopropylsilyl)ethynyl)naphthalen-1-yl)-2-((1-(pyrrolidin-1-ylmethyl)cyclopropyl)methoxy)pyrido[4,3-d]pyrimidin-4-yl)-3,8-diazabicyclo[3.2.1]octane-8-carboxylate

Add tert-butyl (1R,5S)-3-(7-chloro-8-fluoro-2-((1-(pyrrolidin-1-ylmethyl)cyclopropyl)methoxy) pyrido[4,3-d]pyrimidin-4-yl)-3,8-diazabicyclo[3.2.1]octane-8-carboxylate (50 mg, 0.09 mmol), triisopropyl((6-(methoxymethoxy)-8-(4,4,5,5-tetramethyl-1,3,2-dioxaborolan-2-yl)naphthalen-1-yl)ethynyl)silane (57 mg, 0.11 mmol) and cesium carbonate (74 mg, 0.23 mmol) into 1,4-dioxane/water=5/1 (3 mL). Add Pd(dppf)Cl₂ (13 mg, 0.02 mmol), run evacuating and backfilling with argon for 3 times. Heat the reaction at 100° C. for 8 hours. After completion, cooled the reaction to room temperature and add water. Use ethyl acetate to extract the reaction. The organic layer was dried over sodium sulfate and concentrated. Purification with prep-TLC gave off-white solid. (35 mg, yield: 42%)

### Step 11: synthesis of 4-(4-((1R,5S)-3,8-diazabicyclo[3.2.1]octan-3-yl)-8-fluoro-2-((1-(pyrrolidin-1-ylmethyl)cyclopropyl)methoxy)pyrido[4,3-d]pyrimidin-7-yl)-5-ethynylnaphthalen-2-ol

Dissolve tert-butyl (1R,5S)-3-(8-fluoro-7-(3-(methoxymethoxy)-8-((triisopropylsilyl)ethynyl) naphthalen-1-yl)-2-((1-(pyrrolidin-1-ylmethyl)cyclopropyl)methoxy) pyrido[4,3-d]pyrimidin-4-yl)-3,8-diazabicyclo[3.2.1]octane-8-carboxylate (35 mg, 0.04 mmol) in DMF (3 mL). Add CsF (30 mg, 0.20 mmol). Stir the reaction at room temperature for 2 hours. After completion, add water and extract the reaction with ethyl acetate. The organic layer was washed with brine, dried over sodium sulfate and concentrated. The crude product was dissolved in acetonitrile (0.5 mL). Then 4M HCl in 1,4-dioxane (0.5 mL) was added. The mixture was stirred at room temperature for 30 min. After completion, saturated aqueous sodium bicarbonate was added carefully to adjust pH to 7. Use DCM to run extraction. The organic layer was dried over sodium sulfate and concentrated. Purification with prep-TLC (DCM/MeOH(7N NH₃)=10/1) gave light yellow solid (15 mg, yield: 64%). MS m/z: 597.6 [M+H]+

The compounds of Examples 2-77 were prepared by preparation method 1.

| | | | m/z: ES⁺ |
|---|---|---|---|
| Ex. | Compound name | Structural formula | [M + H] |
| 2 | 4-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- ethynyl-6- fluoronaphthalen-2- ol | 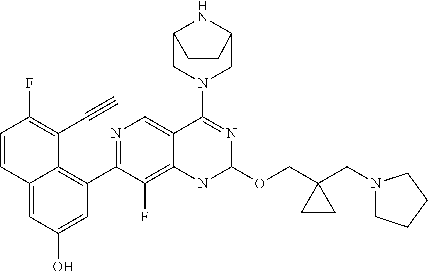 | 602.2 |
| 3 | 4-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- ethyl-6- fluoronaphthalen-2- ol | 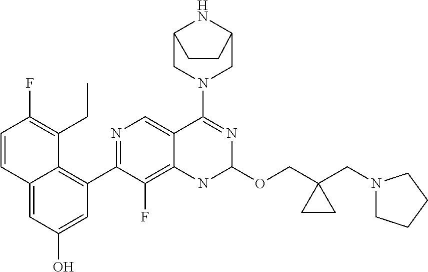 | 601 |
| 4 | 4-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- ethylnaphthalen-2-ol | 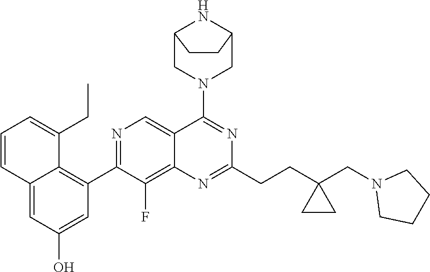 [SMILES: CCc1cccc2cc(O)cc(-c3ncc4c(N5CC6CCC(C5)N6)nc(CCC5(CN6CCCC6)CC5)nc4c3F)c12] | 583 |
| 5 | 4-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5, 6-difluoronaphthalen- 2-ol | 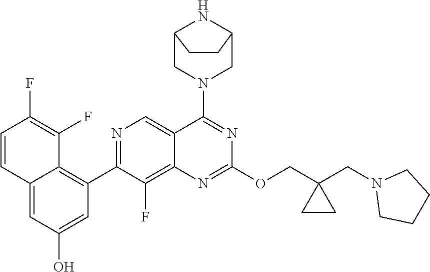 [SMILES: Oc1cc(-c2ncc3c(N4CC5CCC(C4)N5)nc(OCC4(CN5CCCC5)CC4)nc3c2F)c2c(F)c(F)ccc2c1] | 591 |
| 6 | 4-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- fluoronaphthalen-2-ol | 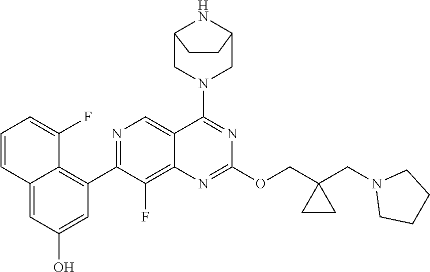 [SMILES: Oc1cc(-c2ncc3c(N4CC5CCC(C4)N5)nc(OCC4(CN5CCCC5)CC4)nc3c2F)c2c(F)cccc2c1] | 573.6 |
| 7 | 1-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7- yl)isoquinolin-3- amine | 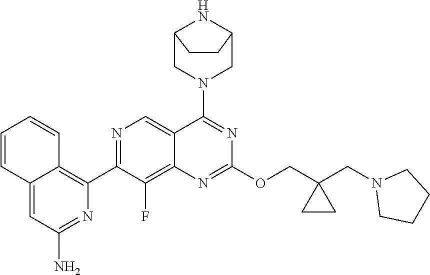 [SMILES: Nc1cc2ccccc2c(-c2ncc3c(N4CC5CCC(C4)N5)nc(OCC4(CN5CCCC5)CC4)nc3c2F)n1] | 555.6 |
| 8 | 6-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-4- methyl-5- (trifluoromethyl) pyridin-2-amine | 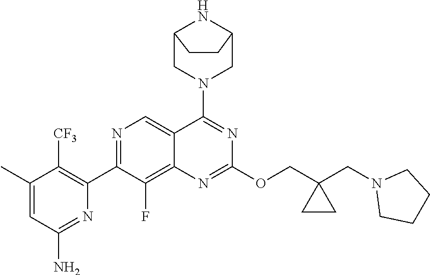 [SMILES: Cc1cc(N)nc(-c2ncc3c(N4CC5CCC(C4)N5)nc(OCC4(CN5CCCC5)CC4)nc3c2F)c1C(F)(F)F] | 587 |
| 9 | 6-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- cyclopropyl-4- methylpyridin-2- amine | 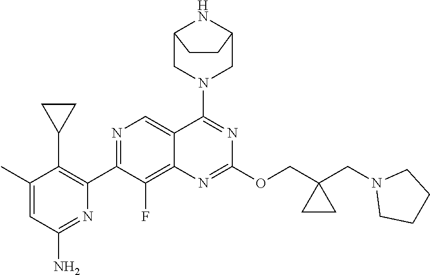 [SMILES: Cc1cc(N)nc(-c2ncc3c(N4CC5CCC(C4)N5)nc(OCC4(CN5CCCC5)CC4)nc3c2F)c1C1CC1] | 559 |
| 10 | 3-(4-((1R,5S)-3,8- diazabicyclo[3.2.1] octan-3-yl)-8-fluoro- 2-((1-(pyrrolidin-1- ylmethyl)cyclopropyl) methoxy)pyrido[4,3- d]pyrimidin-7-yl)-5- chloro-4- cyclopropylphenol | 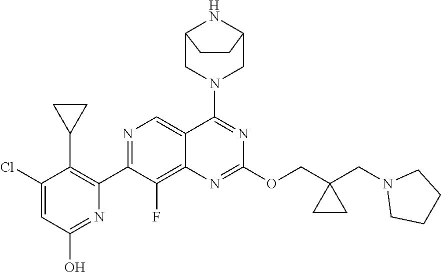 [SMILES: Oc1cc(Cl)c(C2CC2)c(-c2ncc3c(N4CC5CCC(C4)N5)nc(OCC4(CN5CCCC5)CC4)nc3c2F)n1] | 579 |
| 11 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethynylnaphthalen-2- ol | 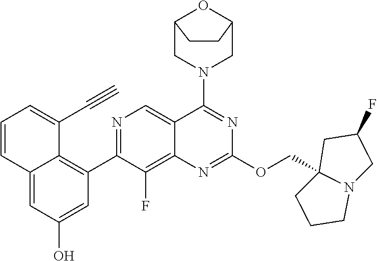 [SMILES: C#Cc1cccc2cc(O)cc(-c3ncc4c(N5CC6CCC(C5)O6)nc(OC[C@@]56CCCN5C[C@H](F)C6)nc4c3F)c12] | 584.6 |
| 12 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethynyl-6- fluoronaphthalen-2- ol | 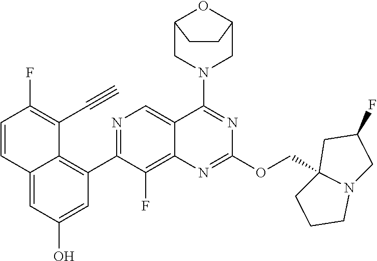 [SMILES: C#Cc1c(F)ccc2cc(O)cc(-c3ncc4c(N5CC6CCC(C5)O6)nc(OC[C@@]56CCCN5C[C@H](F)C6)nc4c3F)c12] | 602.2 |
| 13 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethyl-6- fluoronaphthalen-2- ol | 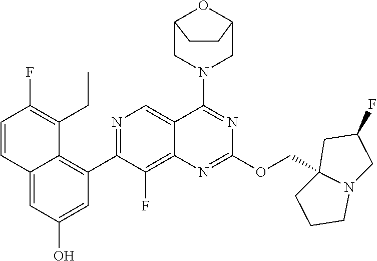 [SMILES: CCc1c(F)ccc2cc(O)cc(-c3ncc4c(N5CC6CCC(C5)O6)nc(OC[C@@]56CCCN5C[C@H](F)C6)nc4c3F)c12] | 606.2 |
| 14 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethylnaphthalen-2-ol | 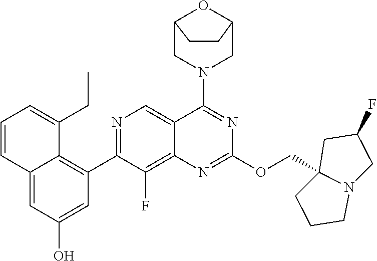 [SMILES: CCc1cccc2cc(O)cc(-c3ncc4c(N5CC6CCC(C5)O6)nc(OC[C@@]56CCCN5C[C@H](F)C6)nc4c3F)c12] | 588.3 |
| 15 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- fluoronaphthalen-2- ol | 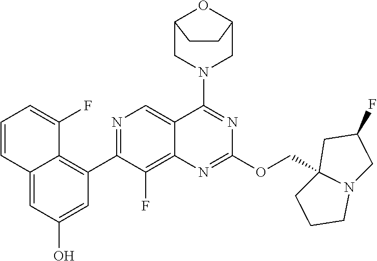 [SMILES: Oc1cc(-c2ncc3c(N4CC5CCC(C4)O5)nc(OC[C@@]45CCCN4C[C@H](F)C5)nc3c2F)c2c(F)cccc2c1] | 578.3 |
| 16 | 4-(4-((1R,5S)-8-oxa- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5,6- difluoronaphthalen-2- ol | 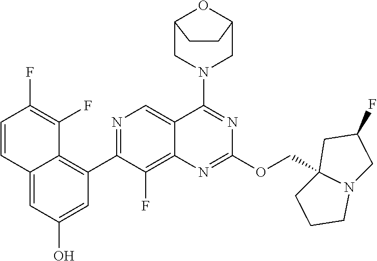 [SMILES: Oc1cc(-c2ncc3c(N4CC5CCC(C4)O5)nc(OC[C@@]45CCCN4C[C@H](F)C5)nc3c2F)c2c(F)c(F)ccc2c1] | 596.2 |
| 17 | 4-(4-((1R,5S)-3-thia- 8-azabicyclo[3.2.1] octan-8-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethynyl-6- fluoronaphthalen-2- ol | 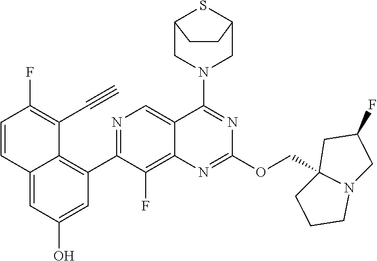 [SMILES: C#Cc1c(F)ccc2cc(O)cc(-c3ncc4c(N5CC6CCC(C5)S6)nc(OC[C@@]56CCCN5C[C@H](F)C6)nc4c3F)c12] | 618.6 |
| 18 | (1R,5S)-8-(7-(8- ethynyl-7-fluoro-3- hydroxynaphthalen- 1-yl)-8-fluoro-2- (((2R,7aS)-2- fluorotetrahydro-1H- pyrrolizin-7a(5H)- yl)methoxy)pyrido [4,3-d]pyrimidin-4- yl)-3-thia-8- azabicyclo[3.2.1] octane 3-oxide | 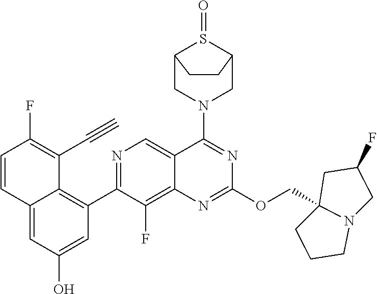 [SMILES: C#Cc1c(F)ccc2cc(O)cc(-c3ncc4c(N5CC6CCC(C5)S6=O)nc(OC[C@@]56CCCN5C[C@H](F)C6)nc4c3F)c12] | 634.3 |
| 19 | (1R,5S)-8-(7-(8- ethynyl-7-fluoro-3- hydroxynaphthalen- 1-yl)-8-fluoro-2- (((2R,7aS)-2- fluorotetrahydro-1H- pyrrolizin-7a(5H)- yl)methoxy)pyrido [4,3-d]pyrimidin-4- yl)-3-thia-8- azabicyclo[3.2.1] octane 3,3-dioxide | 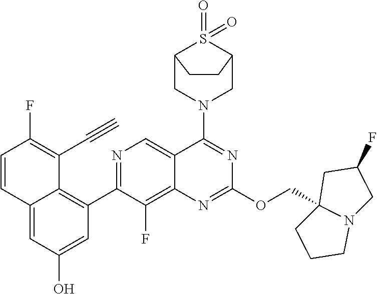 [SMILES: C#Cc1c(F)ccc2cc(O)cc(-c3ncc4c(N5CC6CCC(C5)S6(=O)=O)nc(OC[C@@]56CCCN5C[C@H](F)C6)nc4c3F)c12] | 650.3 |
| 20 | 4-(4-((1R,5S)-8- thia-3-azabicyclo [3.2.1]octan-3-yl)- 8-fluoro-2-(((2R, 7aS)-2- fluorotetrahydro-1H- pyrrolizin-7a(5H)- yl)methoxy)pyrido [4,3-d]pyrimidin-7- yl)-5- ethynylnaphthalen- 2-ol | 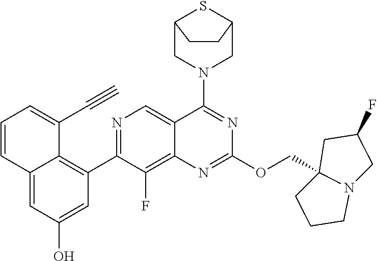 [SMILES: C#Cc1cccc2cc(O)cc(-c3ncc4c(N5CC6CCC(C5)S6)nc(OC[C@@]56CCCN5C[C@H](F)C6)nc4c3F)c12] | 600.7 |
| 21 | 4-(4-((1R,5S)-8-thia- 3-azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethynyl-6- fluoronaphthalen-2- ol | 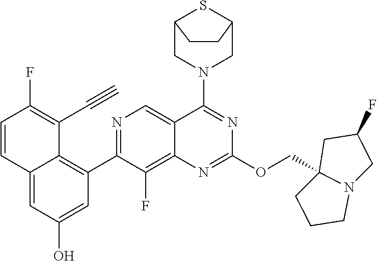 [SMILES: C#Cc1c(F)ccc2cc(O)cc(-c3ncc4c(N5CC6CCC(C5)S6)nc(OC[C@@]56CCCN5C[C@H](F)C6)nc4c3F)c12] | 618 |
| 22 | (1R,5S,8R)-3-(7-(8- ethynyl-7-fluoro-3- hydroxynaphthalen- 1-yl)-8-fluoro-2- (((2R,7aS)-2- fluorotetrahydro- 1H-pyrrolizin-7a(5H)- yl)methoxy)pyrido [4,3-d]pyrimidin-4-yl)- 8-thia-3-azabicyclo [3.2.1]octane 8-oxide | 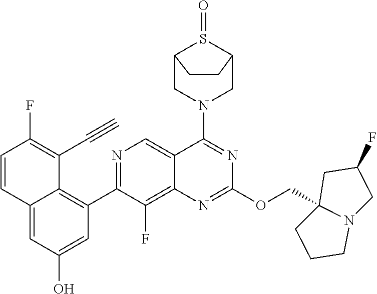 [SMILES: C#Cc1c(F)ccc2cc(O)cc(-c3ncc4c(N5CC6CCC(C5)S6=O)nc(OC[C@@]56CCCN5C[C@H](F)C6)nc4c3F)c12] | 634 |
| 23 | (1R,5S)-3-(7-(8- ethynyl-7-fluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl)-8- thia-3-azabicyclo[3.2.1] octane 8,8-dioxide | 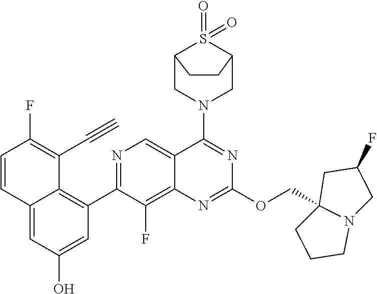 [SMILES: C#Cc1c(F)ccc2cc(O)cc(-c3ncc4c(N5CC6CCC(C5)S6(=O)=O)nc(OC[C@@]56CCCN5C[C@H](F)C6)nc4c3F)c12] | 650 |
| 24 | 4-(4-((1R,5S)-8-thia-3- azabicyclo[3.2.1]octan- 3-yl)-8-fluoro-2-(((2R, 7aS)-2-fluorotetrahydro- 1H-pyrrolizin-7a(5H)- yl)methoxy)pyrido[4,3- d]pyrimidin-7-yl)-5-ethyl- 6-fluoronaphthalen-2-ol | 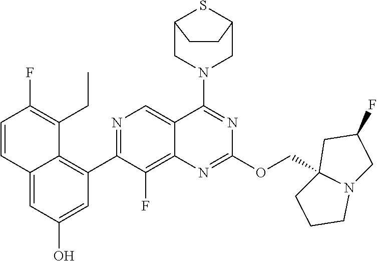 [SMILES: CCc1c(F)ccc2cc(O)cc(-c3ncc4c(N5CC6CCC(C5)S6)nc(OC[C@@]56CCCN5C[C@H](F)C6)nc4c3F)c12] | 622 |
| 25 | 4-(4-((1R,5S)-8-thia-3- azabicyclo[3.2.1]octan- 3-yl)-8-fluoro-2-(((2R, 7aS)-2-fluorotetrahydro- 1H-pyrrolizin-7a(5H)- yl)methoxy)pyrido[4,3- d]pyrimidin-7-yl)-5- ethylnaphthalen-2-ol | 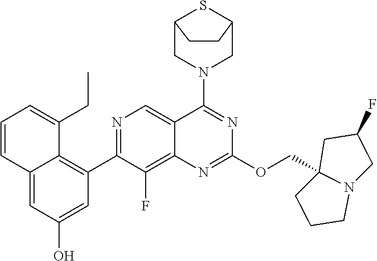 [SMILES: CCc1cccc2cc(O)cc(-c3ncc4c(N5CC6CCC(C5)S6)nc(OC[C@@]56CCCN5C[C@H](F)C6)nc4c3F)c12] | 604.7 |
| 26 | 4-(4-((1R,5S)-8-thia-3- azabicyclo[3.2.1]octan- 3-yl)-8-fluoro-2-(((2R, 7aS)-2-fluorotetrahydro- 1H-pyrrolizin-7a(5H)- yl)methoxy)pyrido[4,3- d]pyrimidin-7-yl)-5- fluoronaphthalen-2-ol | 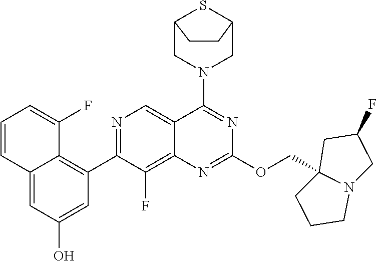 [SMILES: Oc1cc(-c2ncc3c(N4CC5CCC(C4)S5)nc(OC[C@@]45CCCN4C[C@H](F)C5)nc3c2F)c2c(F)cccc2c1] | 594.6 |
| 27 | 4-(4-((1R,5S)-8-thia- 3-azabicyclo[3.2.1] octan-3-yl)-8-fluoro-2- (((2R,7aS)-2- fluorotetrahydro-1H- pyrrolizin-7a(5H)-yl) methoxy)pyrido[4,3- d]pyrimidin-7-yl)-5,6- difluoronaphthalen-2- ol | 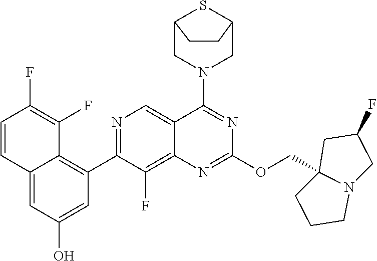 [SMILES: Oc1cc(-c2ncc3c(N4CC5CCC(C4)S5)nc(OC[C@@]45CCCN4C[C@H](F)C5)nc3c2F)c2c(F)c(F)ccc2c1] | 612 |
| 28 | (1R,5S,8R)-3-(7-(7,8- difluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl)-8- thia-3-azabicyclo [3.2.1]octane 8-oxide | 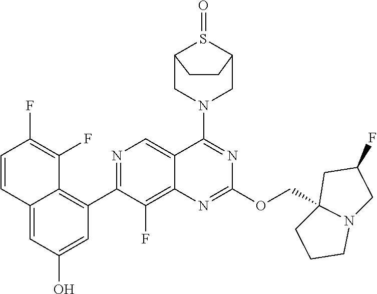 [SMILES: O=S1C2CCC1CN(c1nc(OC[C@@]34CCCN3C[C@H](F)C4)nc3c(F)c(-c4cc(O)cc5ccc(F)c(F)c45)ncc13)C2] | 628.2 |
| 29 | (1R,5S)-3-(7-(7,8- difluoro-3- hydroxynaphthalen- 1-yl)-8-fluoro-2- (((2R,7aS)-2- fluorotetrahydro-1H- pyrrolizin-7a(5H)-yl) methoxy)pyrido[4,3- d]pyrimidin-4-yl)-8- thia-3-azabicyclo [3.2.1 ]octane 8,8- dioxide | 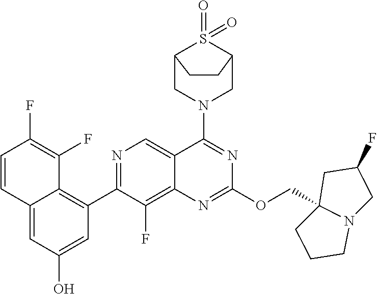 [SMILES: O=S1(=O)C2CCC1CN(c1nc(OC[C@@]34CCCN3C[C@H](F)C4)nc3c(F)c(-c4cc(O)cc5ccc(F)c(F)c45)ncc13)C2] | 644.2 |
| 30 | 4-(4-((1R,5S)-8,8- difluoro-3- azabicyclo[3.2.1] octan-3-yl)-8-fluoro- 2-(((2R,7aS)-2- fluorotetrahydro-1H- pyrrolizin-7a(5H)-yl) methoxy)pyrido[4,3- d]pyrimidin-7-yl)-5- ethynyl-6- fluoronaphthalen-2-ol | 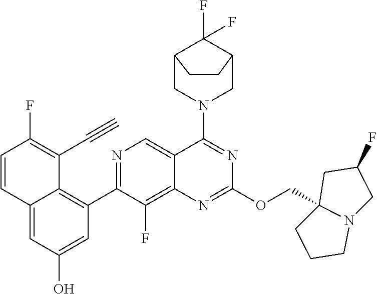 [SMILES: C#Cc1c(F)ccc2cc(O)cc(-c3ncc4c(N5CC6CCC(C5)C6(F)F)nc(OC[C@@]56CCCN5C[C@H](F)C6)nc4c3F)c12] | 636.6 |
| 31 | 4-(4-((1R,5S)-8,8- difluoro-3-azabicyclo [3.2.1]octan-3-yl)- 8-fluoro-2-(((2R,7aS)- 2-fluorotetrahydro- 1H-pyrrolizin-7a(5H)- yl)methoxy)pyrido[4,3- d]pyrimidin-7-yl)-5- ethyl-6- fluoronaphthalen-2-ol | 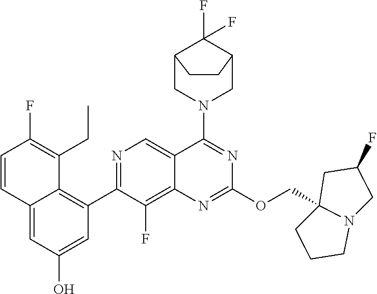 [SMILES: CCc1c(F)ccc2cc(O)cc(-c3ncc4c(N5CC6CCC(C5)C6(F)F)nc(OC[C@@]56CCCN5C[C@H](F)C6)nc4c3F)c12] | 640.6 |
| 32 | 4-(4-((1R,5S)-8,8- difluoro-3-azabicyclo [3.2.1]octan-3-yl)-8- fluoro-2-(((2R,7aS)-2- fluorotetrahydro-1H- pyrrolizin-7a(5H)-yl) methoxy)pyrido[4,3- d]pyrimidin-7-yl)-5- ethynylnaphthalen-2- ol | 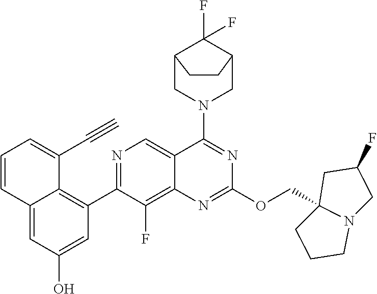 [SMILES: C#Cc1cccc2cc(O)cc(-c3ncc4c(N5CC6CCC(C5)C6(F)F)nc(OC[C@@]56CCCN5C[C@H](F)C6)nc4c3F)c12] | 618.6 |
| 33 | 4-(4-((1R,5S)-8,8- difluoro-3-azabicyclo [3.2.1]octan-3-yl)-8- fluoro-2-(((2R,7aS)- 2-fluorotetrahydro- 1H-pyrrolizin-7a(5H)- yl)methoxy)pyrido[4,3- d]pyrimidin-7-yl)-5,6- difluoronaphthalen-2- ol | 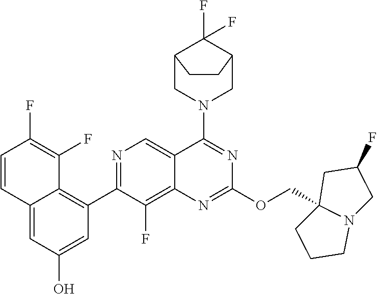 [SMILES: Oc1cc(-c2ncc3c(N4CC5CCC(C4)C5(F)F)nc(OC[C@@]45CCCN4C[C@H](F)C5)nc3c2F)c2c(F)c(F)ccc2c1] | 630 |
| 34 | 5-ethynyl-6-fluoro-4- (8-fluoro-4-((1R,5S, 8R)-8-fluoro-3- azabicyclo [3.2.1]octan-3-yl)-2- (((2R,7aS)-2- fluorotetrahydro-1H- pyrrolizin-7a(5H)-yl) methoxy)pyrido[4,3- d]pyrimidin-7-yl) naphthalen-2-ol | 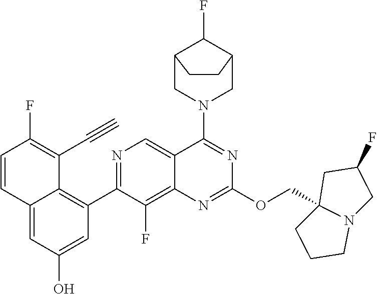 [SMILES: C#Cc1c(F)ccc2cc(O)cc(-c3ncc4c(N5CC6CCC(C5)C6F)nc(OC[C@@]56CCCN5C[C@H](F)C6)nc4c3F)c12] | 618 |
| 35 | 5-ethyl-6-fluoro-4-(8- fluoro-4-((1R,5S,8R)- 8-fluoro-3-azabicyclo [3.2.1]octan-3-yl)-2- (((2R,7aS)-- fluorotetrahydro-1H- pyrrolizin-7a(5H)- yl)methoxy)pyrido[4,3- d]pyrimidin-7-yl) naphthalen-2-ol | 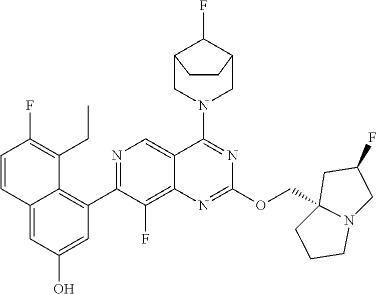 [SMILES: CCc1c(F)ccc2cc(O)cc(-c3ncc4c(N5CC6CCC(C5)C6F)nc(OC[C@@]56CCCN5C[C@H](F)C6)nc4c3F)c12] | 622 |
| 36 | 5-ethynyl-4-(8-fluoro- 4-((1R,5S,8R)-8-fluoro- 3-azabicyclo[3.2.1] octan-3-yl)-2-(((2R,7aS)- 2-fluorotetrahydro- 1H-pyrrolizin-7a(5H)- yl)methoxy)pyrido[4,3- d]pyrimidin-7-yl) naphthalen-2-ol | 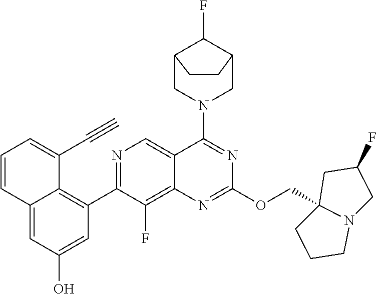 [SMILES: C#Cc1cccc2cc(O)cc(-c3ncc4c(N5CC6CCC(C5)C6F)nc(OC[C@@]56CCCN5C[C@H](F)C6)nc4c3F)c12] | 600 |
| 37 | 5,6-difluoro-4-(8-fluoro- 4-((1R,5S,8R)-8-fluoro- 3-azabicyclo[3.2.1] octan-3-yl)-2-(((2R,7aS)- 2-fluorotetrahydro- 1H-pyrrolizin-7a(5H)- yl)methoxy)pyrido[4,3- d]pyrimidin-7-yl) naphthalen-2-ol | 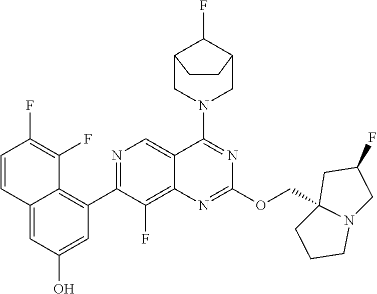 [SMILES: Oc1cc(-c2ncc3c(N4CC5CCC(C4)C5F)nc(OC[C@@]45CCCN4C[C@H](F)C5)nc3c2F)c2c(F)c(F)ccc2c1] | 612 |
| 38 | 1-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-7,8- difluoroisoquinolin-3- amine | 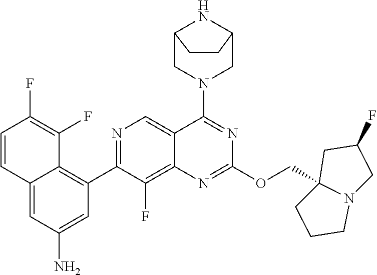 [SMILES: Nc1cc(-c2ncc3c(N4CC5CCC(C4)N5)nc(OC[C@@]45CCCN4C[C@H](F)C5)nc3c2F)c2c(F)c(F)ccc2c1] | 595.25 |
| 39 | 1-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-8- chloro-7- fluoroisoquinolin-3- amine | 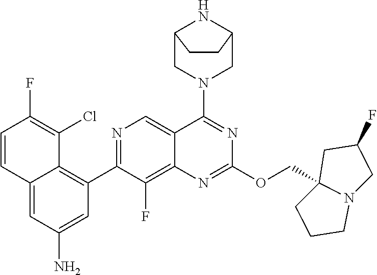 [SMILES: Nc1cc(-c2ncc3c(N4CC5CCC(C4)N5)nc(OC[C@@]45CCCN4C[C@H](F)C5)nc3c2F)c2c(Cl)c(F)ccc2c1] | 611.22 |
| 40 | 1-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-8- ethynyl-7- fluoroisoquinolin-3- amine | 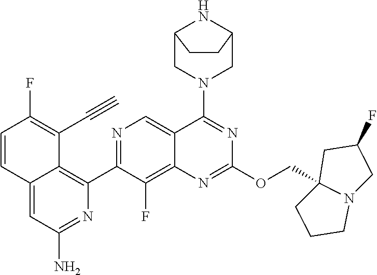 [SMILES: C#Cc1c(F)ccc2cc(N)nc(-c3ncc4c(N5CC6CCC(C5)N6)nc(OC[C@@]56CCCN5C[C@H](F)C6)nc4c3F)c12] | 601.26 |
| 41 | 6-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-4- methyl-5- (trifluoromethyl) pyridin-2-amine | 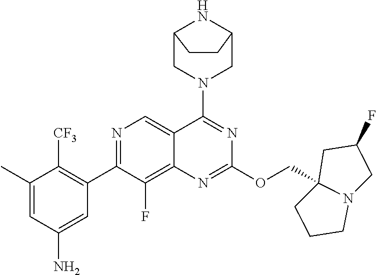 [SMILES: Cc1cc(N)cc(-c2ncc3c(N4CC5CCC(C4)N5)nc(OC[C@@]45CCCN4C[C@H](F)C5)nc3c2F)c1C(F)(F)F] | 591.25 |
| 42 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl) naphthalen-2-amine | 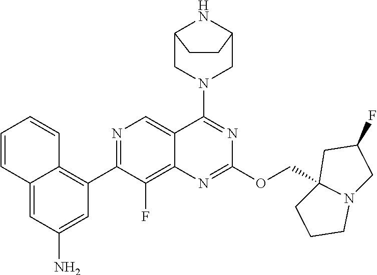 [SMILES: Nc1cc(-c2ncc3c(N4CC5CCC(C4)N5)nc(OC[C@@]45CCCN4C[C@H](F)C5)nc3c2F)c2ccccc2c1] | 558.27 |
| 43 | 4-(4-(3,9-diazabicyclo [3.3.1]nonan-3-yl)-8- fluoro-2-(((2R,7aS)-2- fluorotetrahydro-1H- pyrrolizin-7a(5H)- yl)methoxy)pyrido[4,3- d]pyrimidin-7-yl)-5- ethynyl-6- fluoronaphthalen-2-ol | 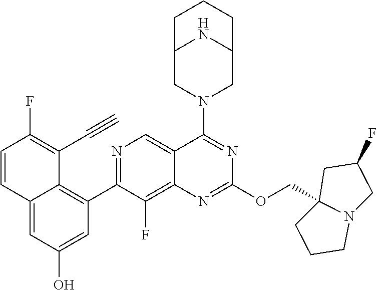 [SMILES: C#Cc1c(F)ccc2cc(O)cc(-c3ncc4c(N5CC6CCCC(C5)N6)nc(OC[C@@]56CCCN5C[C@H](F)C6)nc4c3F)c12] | 615.26 |
| 44 | 4-(4-(3,9-diazabicyclo [3.3.1]nonan-3-yl)-8- fluoro-2-(((2R,7aS)-2- fluorotetrahydro-1H- pyrrolizin-7a(5H)- yl)methoxy)pyrido[4,3- d]pyrimidin-7-yl)-5- ethynylnaphthalen-2-ol | 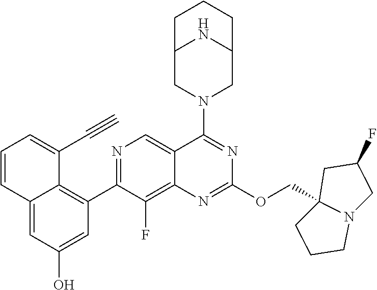 [SMILES: C#Cc1cccc2cc(O)cc(-c3ncc4c(N5CC6CCCC(C5)N6)nc(OC[C@@]56CCCN5C[C@H](F)C6)nc4c3F)c12] | 597.2 |
| 45 | 4-(4-(3,6-diazabicyclo [3.1.1]heptan-3-yl)-8- fluoro-2-(((2R,7aS)-2- fluorotetrahydro-1H- pyrrolizin-7a(5H)- yl)methoxy)pyrido[4,3- d]pyrimidin-7-yl)-5- ethynyl-6- fluoronaphthalen-2-ol | 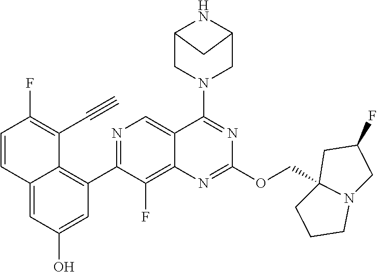 [SMILES: C#Cc1c(F)ccc2cc(O)cc(-c3ncc4c(N5CC6CC(C5)N6)nc(OC[C@@]56CCCN5C[C@H](F)C6)nc4c3F)c12] | 587.23 |
| 46 | 4-(4-(2,5-diazabicyclo [2.2.2]octan-2-yl)-8- fluoro-2-(((2R,7aS)-2- fluorotetrahydro-1H- pyrrolizin-7a(5H)- yl)methoxy)pyrido[4,3- d]pyrimidin-7-yl)-5- ethynyl-6- fluoronaphthalen-2-ol | 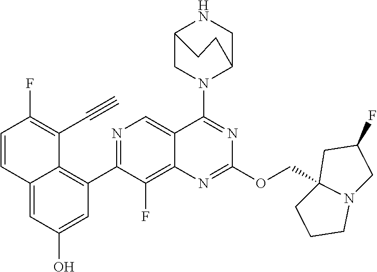 [SMILES: C#Cc1c(F)ccc2cc(O)cc(-c3ncc4c(N5CC6CCC5CN6)nc(OC[C@@]56CCCN5C[C@H](F)C6)nc4c3F)c12] | 601.25 |
| 47 | 5-ethynyl-6-fluoro-4- (8-fluoro-2-(((2R,7aS)- 2-fluorotetrahydro- 1H-pyrrolizin-7a(5H)- yl)methoxy)-4-(1,8- diazaspiro[4.5]decan- 8-yl)pyrido[4,3-d] pyrimidin-7-yl) naphthalen-2-ol | 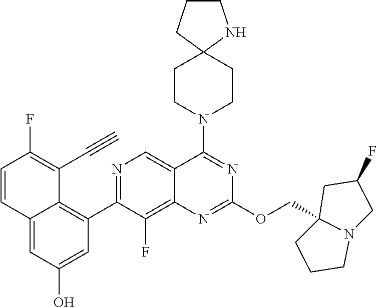 [SMILES: C#Cc1c(F)ccc2cc(O)cc(-c3ncc4c(N5CCC6(CCCN6)CC5)nc(OC[C@@]56CCCN5C[C@H](F)C6)nc4c3F)c12] | 629.28 |
| 48 | 5-ethynyl-4-(8-fluoro- 2-(((2R,7aS)-2- fluorotetrahydro-1H- pyrrolizin-7a(5H)- yl)methoxy)-4-(1,8- diazaspiro[4.5]decan- 8-yl)pyrido[4,3-d] pyrimidin-7-yl) naphthalen-2-ol | 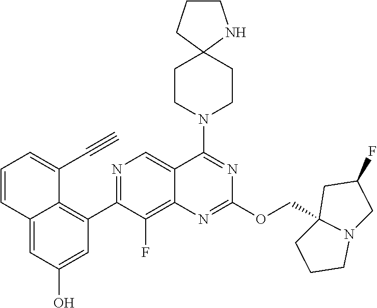 [SMILES: C#Cc1cccc2cc(O)cc(-c3ncc4c(N5CCC6(CCCN6)CC5)nc(OC[C@@]56CCCN5C[C@H](F)C6)nc4c3F)c12] | 611.7 |
| 49 | 5-ethynyl-6-fluoro-4- (8-fluoro-2-(((2R, 7aS)-2- fluorotetrahydro-1H- pyrrolizin-7a(5H)-yl) methoxy)-4-(1- thia-8- azaspiro[4.5]decan-8- yl)pyrido[4,3-d] pyrimidin-7- yl)naphthalen-2-ol | 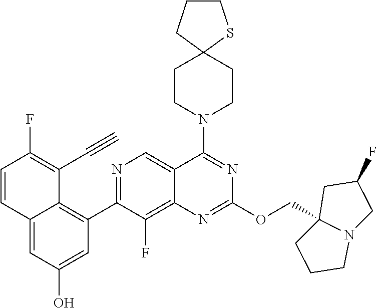 [SMILES: C#Cc1c(F)ccc2cc(O)cc(-c3ncc4c(N5CCC6(CCCS6)CC5)nc(OC[C@@]56CCCN5C[C@H](F)C6)nc4c3F)c12] | 646.24 |
| 50 | 8-(7-(8-ethynyl-7- fluoro-3- hydroxynaphthalen-1- yl)-8-fluoro-2-(((2R, 7aS)-2- fluorotetrahydro-1H- pyrrolizin-7a(5H)-yl) methoxy)pyrido[4,3- d]pyrimidin-4-yl)-1- thia-8-azaspiro[4.5] decane 1,1-dioxide | 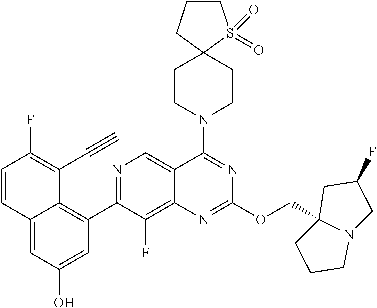 [SMILES: C#Cc1c(F)ccc2cc(O)cc(-c3ncc4c(N5CCC6(CCCS6(=O)=O)CC5)nc(OC[C@@]56CCCN5C[C@H](F)C6)nc4c3F)c12] | 678.23 |
| 51 | 4-(4-(3- aminopyrrolidin-1- yl)-8-fluoro-2-(((2R, 7aS)-2- fluorotetrahydro- 1H-pyrrolizin-7a(5H)- yl)methoxy)pyrido[4,3- d]pyrimidin-7-yl)-5- ethynyl-6- fluoronaphthalen-2-ol | 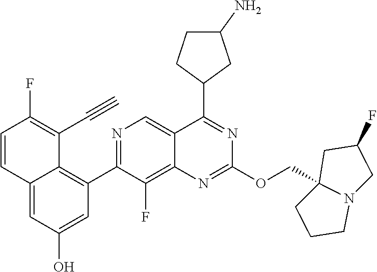 [SMILES: C#Cc1c(F)ccc2cc(O)cc(-c3ncc4c(C5CCC(N)C5)nc(OC[C@@]56CCCN5C[C@H](F)C6)nc4c3F)c12] | 575.23 |
| 52 | 5-ethynyl-6-fluoro-4- (8-fluoro-2-(((2R,7aS)- 2-fluorotetrahydro-1H- pyrrolizin-7a(5H)-yl) methoxy)-4-(octahydro- 6H-pyrrolo[3,4-b] pyridin-6-yl)pyrido[4,3- d]pyrimidin-7- yl)naphthalen-2-ol | 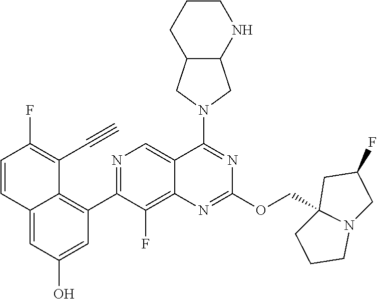 [SMILES: C#Cc1c(F)ccc2cc(O)cc(-c3ncc4c(N5CC6CCCNC6C5)nc(OC[C@@]56CCCN5C[C@H](F)C6)nc4c3F)c12] | 615.26 |
| 53 | 5-ethynyl-4-(8-fluoro- 2-(((2R,7aS)-2- fluorotetrahydro-1H- pyrrolizin-7a(5H)-yl) methoxy)-4-(octahydro- 6H-pyrrolo[3,4-b] pyridin-6-yl)pyrido[4,3- d]pyrimidin-7- yl)naphthalen-2-ol | 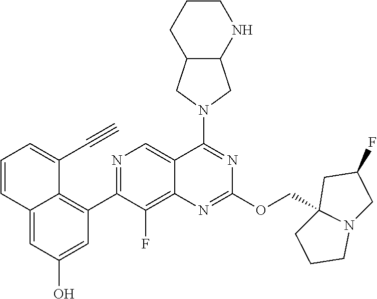 [SMILES: C#Cc1cccc2cc(O)cc(-c3ncc4c(N5CC6CCCNC6C5)nc(OC[C@@]56CCCN5C[C@H](F)C6)nc4c3F)c12] | 597.6 |
| 54 | 5-ethynyl-6-fluoro-4- (8-fluoro-2-(((2R,7aS)- 2-fluorotetrahydro-1H- pyrrolizin-7a(5H)-yl) methoxy)-4- (hexahydropyrrolo[3,4- b]pyrrol-5(1H)-yl)pyrido [4,3-d]pyrimidin-7-yl) naphthalen-2-ol | 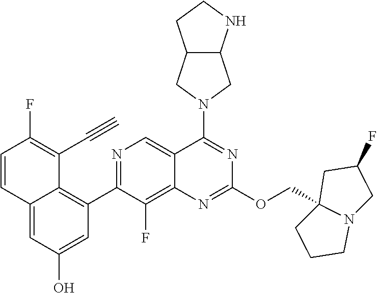 [SMILES: C#Cc1c(F)ccc2cc(O)cc(-c3ncc4c(N5CC6CCNC6C5)nc(OC[C@@]56CCCN5C[C@H](F)C6)nc4c3F)c12] | 601.25 |
| 55 | 4-(4-((3S,4S)-4-amino- 3-methyl-2-oxa-8- azaspiro[4.5]decan-8- yl)-8-fluoro-2-(((2R, 7aS)-2- fluorotetrahydro-1H- pyrrolizin-7a(5H)- yl)methoxy)pyrido[4,3- d]pyrimidin-7-yl)-5- ethynyl-6- fluoronaphthalen-2-ol | 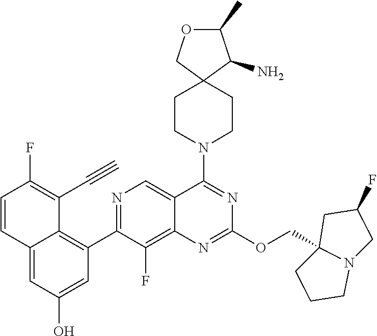 [SMILES: C#Cc1c(F)ccc2cc(O)cc(-c3ncc4c(N5CCC6(CC5)CO[C@@H](C)[C@H]6N)nc(OC[C@@]56CCCN5C[C@H](F)C6)nc4c3F)c12] | 659.29 |
| 56 | 4-(4-((3S,4S)-4-amino- 3-methyl-2-oxa-8- azaspiro[4.5]decan-8- yl)-8-fluoro-2-(((2R, 7aS)-2- fluorotetrahydro-1H- pyrrolizin-7a(5H)- yl)methoxy)pyrido[4,3- d]pyrimidin-7-yl)-5- ethynylnaphthalen-2-ol | 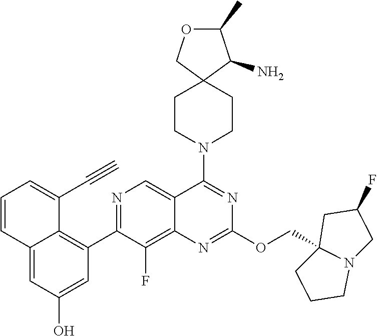 [SMILES: C#Cc1cccc2cc(O)cc(-c3ncc4c(N5CCC6(CC5)CO[C@@H](C)[C@H]6N)nc(OC[C@@]56CCCN5C[C@H](F)C6)nc4c3F)c12] | 641.7 |
| 57 | 4-(4-((3S,4S)-4-amino- 3-methyl-2-oxa-8- azaspiro[4.5]decan-8- yl)-8-fluoro-2-(((2R, 7aS)-2- fluorotetrahydro-1H- pyrrolizin-7a(5H)- yl)methoxy)pyrido[4,3- d]pyrimidin-7-yl)-5,6- difluoronaphthalen-2-ol | 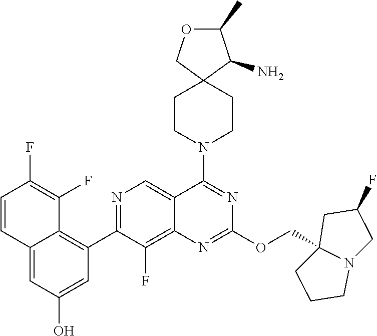 [SMILES: C[C@@H]1OCC2(CCN(c3nc(OC[C@@]45CCCN4C[C@H](F)C5)nc4c(F)c(-c5cc(O)cc6ccc(F)c(F)c56)ncc34)CC2)[C@@H]1N] | 653.6 |
| 58 | 5-ethynyl-6-fluoro-4- (8-fluoro-2-(((2R,7aS)- 2-fluorotetrahydro-1H- pyrrolizin-7a(5H)-yl) methoxy)-4-(3- (methylamino)pyrrolidin- 1-yl)pyrido[4,3-d] pyrimidin-7- yl)naphthalen-2-ol | 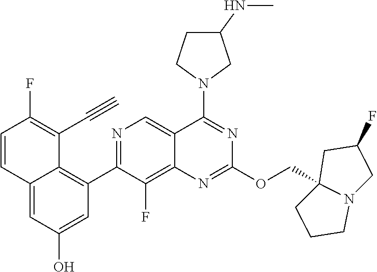 [SMILES: C#Cc1c(F)ccc2cc(O)cc(-c3ncc4c(N5CCC(NC)C5)nc(OC[C@@]56CCCN5C[C@H](F)C6)nc4c3F)c12] | 589.4 |
| 59 | N-(1-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl) pyrrolidin-3-yl) methanesulfonamide | 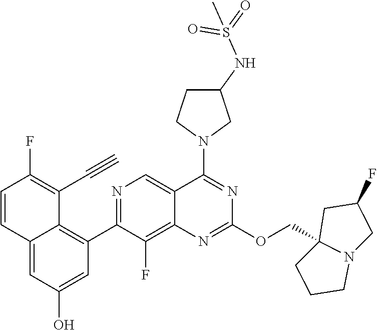 [SMILES: C#Cc1c(F)ccc2cc(O)cc(-c3ncc4c(N5CCC(NS(C)(=O)=O)C5)nc(OC[C@@]56CCCN5C[C@H](F)C6)nc4c3F)c12] | 653.21 |
| 60 | (1R,5S,8R)-3-(7-(8- ethynyl-7-fluoro-3- hydroxynaphthalen- 1-yl)-8-fluoro-2- (((2R,7aS)-2- fluorotetrahydro-1H- pyrrolizin-7a(5H)- yl)methoxy)pyrido[4,3- d]pyrimidin-4-yl)-3- azabicyclo[3.2.1]octan- 8-ol | 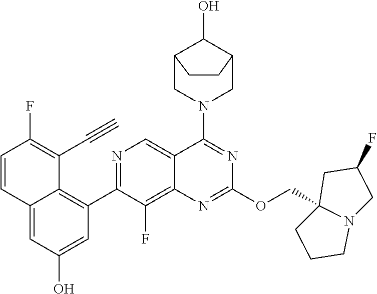 [SMILES: C#Cc1c(F)ccc2cc(O)cc(-c3ncc4c(N5CC6CCC(C5)C6O)nc(OC[C@@]56CCCN5C[C@H](F)C6)nc4c3F)c12] | 616.25 |
| 61 | (1R,5S,8R)-3-(7-(8- ethynyl-3- hydroxynaphthalen-1- yl)-8-fluoro-2-(((2R, 7aS)-2- fluorotetrahydro-1H- pyrrolizin-7a(5H)-yl) methoxy)pyrido[4,3- d]pyrimidin-4-yl)-3- azabicyclo[3.2.1] octan-8-ol | 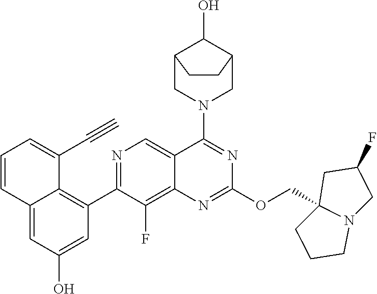 [SMILES: C#Cc1cccc2cc(O)cc(-c3ncc4c(N5CC6CCC(C5)C6O)nc(OC[C@@]56CCCN5C[C@H](F)C6)nc4c3F)c12] | 598.26 |
| 62 | (1R,5S,8S)-3-(7-(8- ethynyl-7-fluoro-3- hydroxynaphthalen- 1-yl)-8-fluoro-2-(((2R, 7aS)-2- fluorotetrahydro-1H- pyrrolizin-7a(5H)- yl)methoxy)pyrido[4,3- d]pyrimidin-4-yl)-3- azabicyclo[3.2.1] octane-8-carbonitrile | 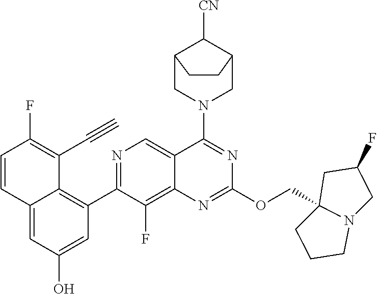 [SMILES: C#Cc1c(F)ccc2cc(O)cc(-c3ncc4c(N5CC6CCC(C5)C6C#N)nc(OC[C@@]56CCCN5C[C@H](F)C6)nc4c3F)c12] | 625.25 |
| 63 | 4-(4-((1R,5S,8R)-8- amino-3-azabicyclo [3.2.1]octan-3-yl)-8- fluoro-2-(((2R,7aS)-2- fluorotetrahydro-1H- pyrrolizin-7a(5H)-yl) methoxy)pyrido[4,3- d]pyrimidin-7-yl)-5- ethynyl-6- fluoronaphthalen-2-ol | 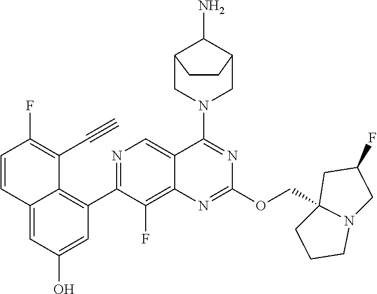 [SMILES: C#Cc1c(F)ccc2cc(O)cc(-c3ncc4c(N5CC6CCC(C5)C6N)nc(OC[C@@]56CCCN5C[C@H](F)C6)nc4c3F)c12] | 615.26 |
| 64 | 5-ethynyl-6-fluoro-4- (8-fluoro-2-(((2R,7aS)- 2-fluorotetrahydro- 1H-pyrrolizin-7a(5H)- yl)methoxy)-4-((1R,5S, 8R)-8-(methylthio)-3- azabicyclo[3.2.1]octan- 3-yl)pyrido[4,3-d] pyrimidin-7-yl) naphthalen-2-ol | 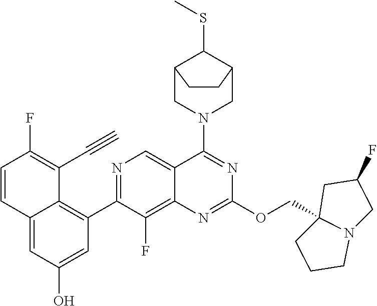 [SMILES: C#Cc1c(F)ccc2cc(O)cc(-c3ncc4c(N5CC6CCC(C5)C6SC)nc(OC[C@@]56CCCN5C[C@H](F)C6)nc4c3F)c12] | 646.24 |
| 65 | 5-ethynyl-6-fluoro-4- (8-fluoro-2-(((2R,7aS)- 2-fluorotetrahydro- 1H-pyrrolizin-7a(5H)- yl)methoxy)-4-((1R,5S, 8R)-8-(methylsulfinyl)- 3-azabicyclo[3.2.1] octan-3-yl)pyrido[4,3- d]pyrimidin-7-yl) naphthalen-2-ol | 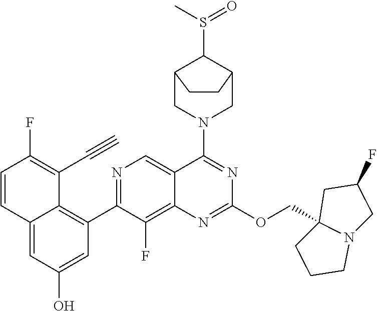 [SMILES: C#Cc1c(F)ccc2cc(O)cc(-c3ncc4c(N5CC6CCC(C5)C6S(C)=O)nc(OC[C@@]56CCCN5C[C@H](F)C6)nc4c3F)c12] | 662.23 |
| 66 | 5-ethynyl-6-fluoro-4- (8-fluoro-2-(((2R,7aS)- 2-fluorotetrahydro- 1H-pyrrolizin-7a(5H)- yl)methoxy)-4-((1R,5S, 8R)-8-(methylsulfonyl)- 3-azabicyclo[3.2.1] octan-3-yl)pyrido[4,3- d]pyrimidin-7-yl) naphthalen-2-ol | 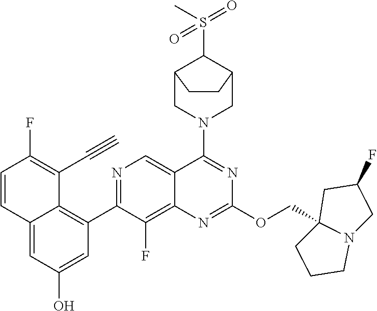 [SMILES: C#Cc1c(F)ccc2cc(O)cc(-c3ncc4c(N5CC6CCC(C5)C6S(C)(=O)=O)nc(OC[C@@]56CCCN5C[C@H](F)C6)nc4c3F)c12] | 678.23 |
| 67 | 5-ethynyl-6-fluoro-4- (8-fluoro-2-(((2R,7aS)- 2-fluorotetrahydro- 1H-pyrrolizin-7a(5H)- yl)methoxy)-4-((1R,5S, 8R)-8- ((trifluoromethyl) sulfonyl)-3- azabicyclo[3.2.1] octan-3-yl)pyrido[4,3- d]pyrimidin-7- yl)naphthalen-2-ol | 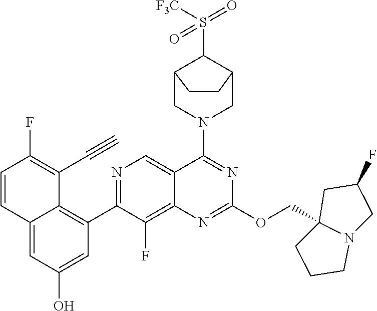 [SMILES: C#Cc1c(F)ccc2cc(O)cc(-c3ncc4c(N5CC6CCC(C5)C6S(=O)(=O)C(F)(F)F)nc(OC[C@@]56CCCN5C[C@H](F)C6)nc4c3F)c12] | 732.20 |
| 68 | 1-(7-(8-ethynyl-7- fluoro-3- hydroxynaphthalen-1- yl)-8-fluoro-2-(((2R, 7aS)-2- fluorotetrahydro-1H- pyrrolizin-7a(5H)-yl) methoxy)pyrido[4,3- d]pyrimidin-4- yl)piperidine-4- carbonitrile | 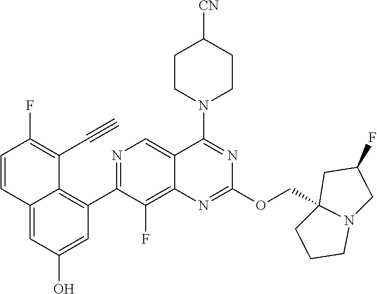 [SMILES: C#Cc1c(F)ccc2cc(O)cc(-c3ncc4c(N5CCC(C#N)CC5)nc(OC[C@@]56CCCN5C[C@H](F)C6)nc4c3F)c12] | 599.23 |
| 69 | 1-(7-(8-ethynyl-7- fluoro-3- hydroxynaphthalen-1- yl)-8-fluoro-2-(((2R, 7aS)-2- fluorotetrahydro-1H- pyrrolizin-7a(5H)-yl) methoxy)pyrido[4,3- d]pyrimidin-4-yl) azetidine-3- carbonitrile | 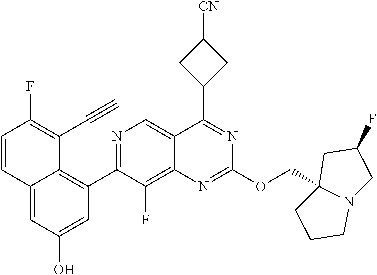 [SMILES: C#Cc1c(F)ccc2cc(O)cc(-c3ncc4c(C5CC(C#N)C5)nc(OC[C@@]56CCCN5C[C@H](F)C6)nc4c3F)c12] | 571.20 |
| 70 | 5-ethynyl-6-fluoro-4- (8-fluoro-2-(((2R, 7aS)-2- fluorotetrahydro- 1H-pyrrolizin-7a(5H)- yl)methoxy)-4- (2,6-diazaspiro[3.3] heptan-2-yl)pyrido[4,3- d]pyrimidin-7- yl)naphthalen-2-ol | 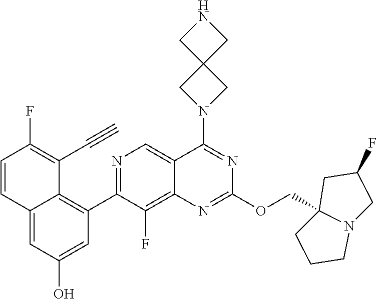 [SMILES: C#Cc1c(F)ccc2cc(O)cc(-c3ncc4c(N5CC6(CNC6)C5)nc(OC[C@@]56CCCN5C[C@H](F)C6)nc4c3F)c12] | 587.23 |
| 71 | 5-ethynyl-6-fluoro-4- (8-fluoro-2-(((2R, 7aS)-2- fluorotetrahydro-1H- pyrrolizin-7a(5H)-yl) methoxy)-4- (quinuclidin-4-yl) pyrido[4,3-d] pyrimidin-7-yl) naphthalen-2-ol | 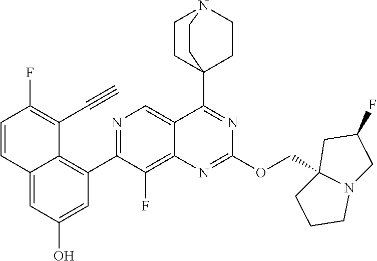 [SMILES: C#Cc1c(F)ccc2cc(O)cc(-c3ncc4c(C56CCN(CC5)CC6)nc(OC[C@@]56CCCN5C[C@H](F)C6)nc4c3F)c12] | 600.25 |
| 72 | 6-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-5- cyclopropyl-4- methylpyridin-2-amine | 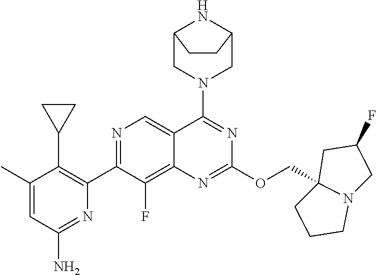 [SMILES: Cc1cc(N)nc(-c2ncc3c(N4CC5CCC(C4)N5)nc(OC[C@@]45CCCN4C[C@H](F)C5)nc3c2F)c1C1CC1] | 563.30 |
| 73 | 2-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)pyrido[4,3- d]pyrimidin-7-yl)-1, 1a, 6,6a- tetrahydrocyclopropa [a]inden-4-ol | 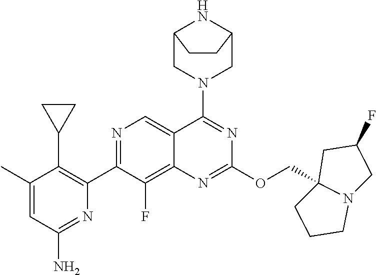 [SMILES: Cc1cc(N)nc(-c2ncc3c(N4CC5CCC(C4)N5)nc(OC[C@@]45CCCN4C[C@H](F)C5)nc3c2F)c1C1CC1] | 561.4 |
| 74 | 4-(4-((1R,5S)-3,8- diazabicyclo [3.2.1] octan-3-yl)-8- fluoro-2-((1- methylhexahydrocyclo- penta[b]pyrrol-3a(1H)- yl)methoxy)pyrido[4,3- d]pyrimidin-7-yl)-5- ethynyl-6- fluoronaphthalen-2-ol | 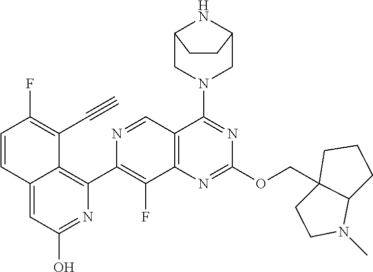 [SMILES: C#Cc1c(F)ccc2cc(O)nc(-c3ncc4c(N5CC6CCC(C5)N6)nc(OCC56CCCC5N(C)CC6)nc4c3F)c12] | 597.4 |
| 75 | 4-(4-((1R,5S)-3,8- diazabicyclo[3.2.1] octan-3-yl)-8-fluoro- 2-((1-methyltetrahydro- 1H-furo[3,4-b]pyrrol- 3a(4H)-yl)methoxy) pyrido[4,3-d]pyrimidin- 7-yl)-5-ethynyl-6- fluoronaphthalen-2-ol | 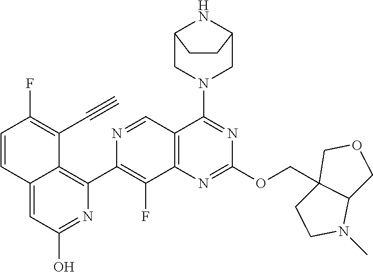 [SMILES: C#Cc1c(F)ccc2cc(O)nc(-c3ncc4c(N5CC6CCC(C5)N6)nc(OCC56CCN(C)C5COC6)nc4c3F)c12] | 599.4 |
| 76 | 4-(4-((1R,5S)-3,8- diazabicyclo[3.2.1]octan- 3-yl)-8-fluoro-2-((3- methyl-3-azabicyclo [3.1.0]hexan-1- yl)methoxy)pyrido[4,3- d]pyrimidin-7-yl)-5- ethynyl-6- fluoronaphthalen-2-ol | 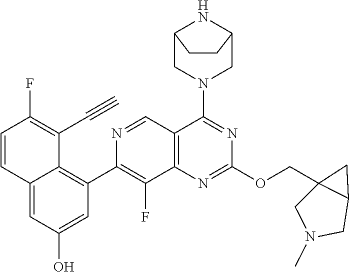 [SMILES: C#Cc1c(F)ccc2cc(O)cc(-c3ncc4c(N5CC6CCC(C5)N6)nc(OCC56CC5CN(C)C6)nc4c3F)c12] | 569.4 |
| 77 | 4-(4-((1R,5S)-3,8- diazabicyclo[3.2.1] octan-3-yl)-8-fluoro- 2-((2-methyl-2- azabicyclo[3.1.0]hexan- 1-yl)methoxy)pyrido [4,3-d]pyrimidin-7-yl)- 5-ethynyl-6- fluoronaphthalen-2-ol | 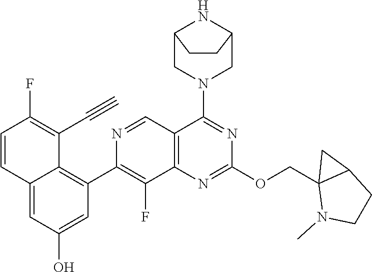 [SMILES: C#Cc1c(F)ccc2cc(O)cc(-c3ncc4c(N5CC6CCC(C5)N6)nc(OCC56CC5CCN6C)nc4c3F)c12] | 569.4 |

### Example 78: synthesis of 4-(4-((1R,5S)-8-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol

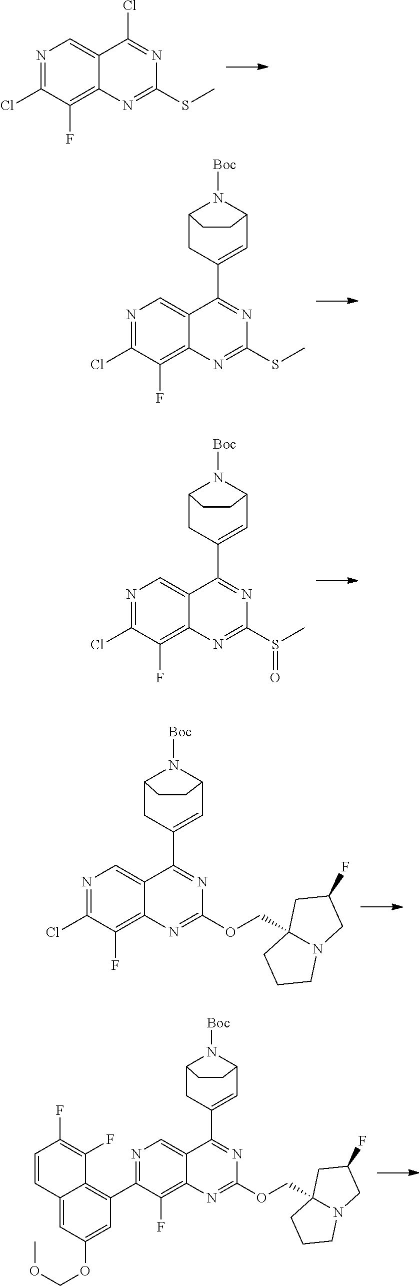 [SMILES: CSc1nc(Cl)c2cnc(Cl)c(F)c2n1 ; CSc1nc(C2=CC3CCC(C2)N3C(=O)OC(C)(C)C)c2cnc(Cl)c(F)c2n1 ; CS(=O)c1nc(C2=CC3CCC(C2)N3C(=O)OC(C)(C)C)c2cnc(Cl)c(F)c2n1 ; CC(C)(C)OC(=O)N1C2C=C(c3nc(OC[C@@]45CCCN4C[C@H](F)C5)nc4c(F)c(Cl)ncc34)CC1CC2 ; COCOc1cc(-c2ncc3c(C4=CC5CCC(C4)N5C(=O)OC(C)(C)C)nc(OC[C@@]45CCCN4C[C@H](F)C5)nc3c2F)c2c(F)c(F)ccc2c1]

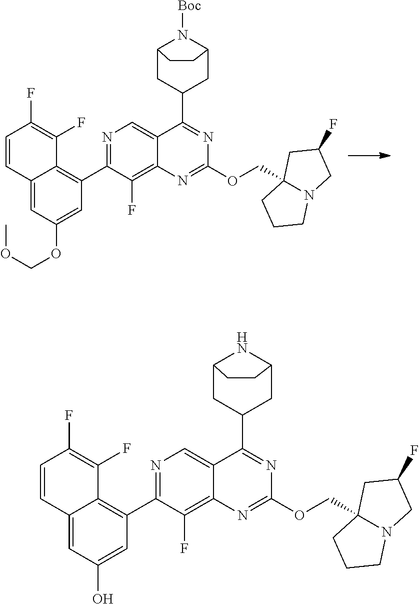 [SMILES: COCOc1cc(-c2ncc3c(C4CC5CCC(C4)N5C(=O)OC(C)(C)C)nc(OC[C@@]45CCCN4C[C@H](F)C5)nc3c2F)c2c(F)c(F)ccc2c1 ; Oc1cc(-c2ncc3c(C4CC5CCC(C4)N5)nc(OC[C@@]45CCCN4C[C@H](F)C5)nc3c2F)c2c(F)c(F)ccc2c1]

### Step 1: synthesis of tert-butyl (1S,5R)-3-(7-chloro-8-fluoro-2-(methylthio)pyrido[4,3-d]pyrimidin-4-yl)-8-azabicyclo[3.2.1]oct-2-ene-8-carboxylate

Add 4,7-dichloro-8-fluoro-2-(methylthio)pyrido[4,3-d]pyrimidine (500 mg, 1.89 mmol), tert-butyl (1S,5R)-3-(4,4,5,5-tetramethyl-1,3,2-dioxaborolan-2-yl)-8-azabicyclo[3.2.1]oct-2-ene-8-carboxylate (826 mg, 2.46 mmol), potassium carbonate (653 mg, 4.73 mmol) and Pd(dppf)Cl₂ (138 mg, 0.19 mmol) to 1,4-dioxane/water=5/1 (10 mL). Run evacuating and backfilling with argon. Heat the reaction at 100° C. and stir for 12 hours. After completion, cool the reaction to room temperature, add water, use ethyl acetate to run extraction. The organic layer was dried over sodium sulfate and concentrated. Purification by prep-TLC gave off-white solid (300 mg, yield: 36.3%). MS m/z: 437 [M+H]+.

### Step 2: synthesis of tert-butyl (1S,5R)-3-(7-chloro-8-fluoro-2-(methylsulfinyl)pyrido[4,3-d]pyrimidin-4-yl)-8-azabicyclo[3.2.1]oct-2-ene-8-carboxylate

Dissolve tert-butyl (1S,5R)-3-(7-chloro-8-fluoro-2-(methylthio)pyrido[4,3-d]pyrimidin-4-yl)-8-azabicyclo[3.2.1]oct-2-ene-8-carboxylate (300 mg, 0.69 mmol) in DCM (10 mL), add m-CPBA (168 mg, 0.82 mmol, 85%) with cooling by ice-water bath. Stir the reaction in ice-water bath for 10 min. After completion, the reaction was quenched by addition of saturated aqueous sodium thiosulfate. The reaction was extracted with DCM. The organic layer was washed with saturated aqueous sodium bicarbonate and brine, dried over sodium sulfate. Concentration gave off-white solid which was used directly in next step (310 mg, yield: 99%).

### Step 3: synthesis of tert-butyl (1S,5R)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]oct-2-ene-8-carboxylate

Dissolve tert-butyl (1S,5R)-3-(7-chloro-8-fluoro-2-(methylsulfinyl)pyrido[4,3-d]pyrimidin-4-yl)-8-azabicyclo[3.2.1]oct-2-ene-8-carboxylate (310 mg, 0.68 mmol) and ((2R,7aS)-2-fluorotetrahydro-1H-pyrrolizin-7a(5H)-yl)methanol (142 mg, 0.89 mmol) in anhydrous toluene (10 mL). Add sodium tert-butoxide (79 mg, 0.82 mmol) under cooling with ice-water bath. Stir the reaction for 30 min under cooling with ice-water bath. After completion, add cooled water to quench the reaction. The reaction was extracted with ethyl acetate. The organic layer was washed with brine, dried over sodium sulfate and concentrated. Purification with column chromatography gave off-white solid (243 mg, yield: 64%). MS m/z: 548 [M+H]+

### Step 4: synthesis of tert-butyl (1S,5R)-3-(7-(7,8-difluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]oct-2-ene-8-carboxylate

Dissolve tert-butyl (1S,5R)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]oct-2-ene-8-carboxylate (50 mg, 0.09 mmol), 2-(7,8-difluoro-3-(methoxymethoxy)naphthalen-1-yl)-4,4,5,5-tetramethyl-1,3,2-dioxaborolane (46 mg, 0.13 mmol), cesium carbonate (82 mg, 0.25 mmol) and Pd(dppf)Cl₂ (8 mg, 0.01 mmol) in 1,4-dioxane/water=5/1 (3 mL). Run evacuating and backfilling with nitrogen, heat the reaction at 100° C. and stir for 12 hours. After completion, the reaction was cooled to room temperature and water was added. Ethyl acetate was used for extraction, the organic layer was dried over sodium sulfate and concentrated. Purification by prep-TLC gave off-white solid (30 mg, yield: 44%). MS m/z: 736 [M+H]+

### Step 5: synthesis of tert-butyl (1R,5S)-3-(7-(7,8-difluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]octane-8-carboxylate

Dissolve tert-butyl (1S,5R)-3-(7-(7,8-difluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]oct-2-ene-8-carboxylate (30 mg, 0.04 mmol) in methanol (5 mL). Add 10% Pd\C (50 mg) under nitrogen atmosphere. Run evacuating and backfilling with hydrogen. Stir the reaction at room temperature for 3 hours. After completion, remove Pd\C by filtering. The following concentration gave off-white solid which was used directly in next step (30 mg, yield: 100%). MS m/z: 738 [M+H]+

### Step 6: synthesis of 4-(4-((1R,5S)-8-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol

Dissolve tert-butyl (1R,5S)-3-(7-(7,8-difluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-azabicyclo[3.2.1]octane-8-carboxylate (30 mg, 0.04 mmol) in acetonitrile (1 mL). Then add a solution of HCl in 1,4-dioxane (1 mL, 4 M). Stir the reaction at room temperature for 30 min. After completion, add aqueous sodium bicarbonate carefully to adjust pH to 7. The mixture was extracted with DCM. The organic layer was dried over sodium sulfate and concentrated. Purification with prep-TLC (DCM/MeOH(7N NH₃)=10/1) gave light yellow solid (15 mg, yield: 62%). MS m/z: 594 [M+H]⁺.

The compounds of Examples 79-88 were prepared by preparation method 78.

| | | | m/z: |
|---|---|---|---|
| Ex. | Compound name | Structural formula | ES⁺[M + H] |
| 79 | 4-(4-((1R,5S)-8- azabicyclo[3.2.1] octan-3-yl)-8-fluoro- 2-(((2R,7aS)-2- fluorotetrahydro- 1H-pyrrolizin- 7a(5H)-yl)methoxy) pyrido[4,3-d] pyrimidin-7-yl)-5- ethynyl-6- fluoronaphthalen-2- ol | 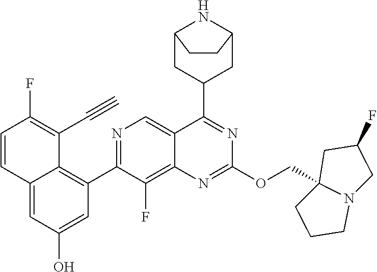 [SMILES: C#Cc1c(F)ccc2cc(O)cc(-c3ncc4c(C5CC6CCC(C5)N6)nc(OC[C@@]56CCCN5C[C@H](F)C6)nc4c3F)c12] | 600.2 |
| 80 | 4-(4-((1R,5S)-8- azabicyclo[3.2.1] octan-3-yl)-8-fluoro- 2-(((2R,7aS)-2- fluorotetrahydro- 1H-pyrrolizin- 7a(5H)-yl)methoxy) pyrido[4,3-d] pyrimidin-7-yl)-5- ethyl-6- fluoronaphthalen-2- ol | 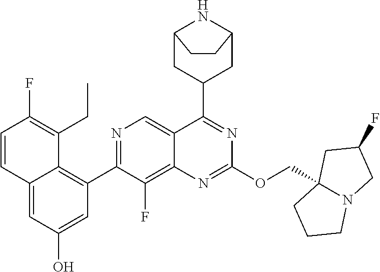 [SMILES: CCc1c(F)ccc2cc(O)cc(-c3ncc4c(C5CC6CCC(C5)N6)nc(OC[C@@]56CCCN5C[C@H](F)C6)nc4c3F)c12] | 604.2 |
| 81 | 4-(4-((1R,5S)-8- azabicyclo[3.2.1] octan-3-yl)-8-fluoro- 2-(((2R,7aS)-2- fluorotetrahydro- 1H-pyrrolizin-7a(5H)- yl)methoxy)pyrido [4,3-d]pyrimidin-7- yl)-5-ethylnaphthalen- 2-ol | 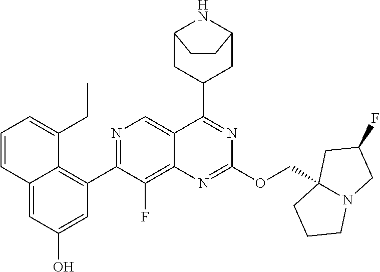 [SMILES: CCc1cccc2cc(O)cc(-c3ncc4c(C5CC6CCC(C5)N6)nc(OC[C@@]56CCCN5C[C@H](F)C6)nc4c3F)c12] | 586.3 |
| 82 | 4-(4-((1R,5S)-8- azabicyclo[3.2.1] octan-3-yl)-8-fluoro- 2-(((2R,7aS)-2- fluorotetrahydro-1H- pyrrolizin-7a(5H)- yl)methoxy)pyrido [4,3-d]pyrimidin-7- yl)-5- fluoronaphthalen-2- ol | 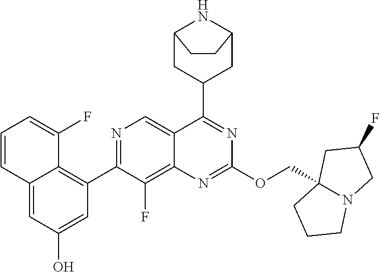 [SMILES: Oc1cc(-c2ncc3c(C4CC5CCC(C4)N5)nc(OC[C@@]45CCCN4C[C@H](F)C5)nc3c2F)c2c(F)cccc2c1] | 576.6 |
| 83 | 4-(4-((1R,5S)-8- azabicyclo[3.2.1] octan-3-yl)-8-fluoro- 2-(((2R,7aS)-2- fluorotetrahydro-1H- pyrrolizin-7a(5H)- yl)methoxy)pyrido [4,3-d]pyrimidin-7- yl)-5- ethynylnaphthalen- 2-ol | 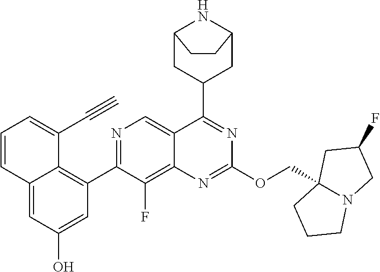 [SMILES: C#Cc1cccc2cc(O)cc(-c3ncc4c(C5CC6CCC(C5)N6)nc(OC[C@@]56CCCN5C[C@H](F)C6)nc4c3F)c12] | 582.4 |
| 84 | 1-(3-(7-(8-ethynyl- 7-fluoro-3- hydroxynaphthalen- 1-yl)-8-fluoro-2- (((2R,7aS)-2- fluorotetrahydro-1H- pyrrolizin-7a(5H)-yl) methoxy)pyrido[4,3- d]pyrimidin- 4-yl) pyrrolidin-1-yl)-2- hydroxyethan-1-one | 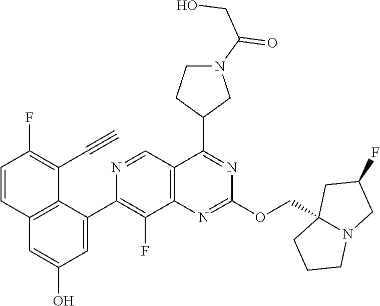 [SMILES: C#Cc1c(F)ccc2cc(O)cc(-c3ncc4c(C5CCN(C(=O)CO)C5)nc(OC[C@@]56CCCN5C[C@H](F)C6)nc4c3F)c12] | 618.4 |
| 85 | 1-(3-(7-(7,8-difluoro- 3-hydroxynaphthalen- 1-yl)-8-fluoro-2- (((2R,7aS)-2- fluorotetrahydro-1H- pyrrolizin-7a(5H)-yl) methoxy)pyrido[4,3- d]pyrimidin-4-yl) pyrrolidin-1-yl)-2- hydroxyethan-1-one | 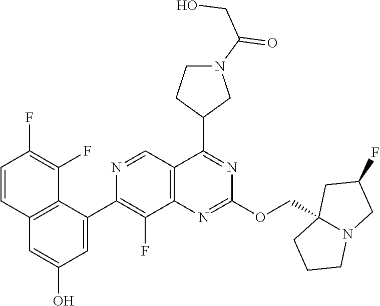 [SMILES: O=C(CO)N1CCC(c2nc(OC[C@@]34CCCN3C[C@H](F)C4)nc3c(F)c(-c4cc(O)cc5ccc(F)c(F)c45)ncc23)C1] | 612.3 |
| 86 | 1-(3-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl)-1H- pyrrol-1-yl)-2- hydroxyethan-1-one | 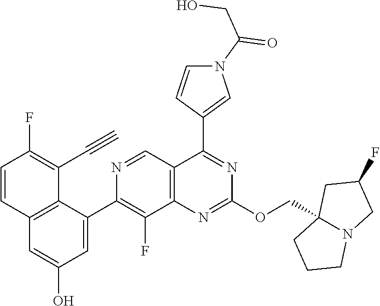 [SMILES: C#Cc1c(F)ccc2cc(O)cc(-c3ncc4c(-c5ccn(C(=O)CO)c5)nc(OC[C@@]56CCCN5C[C@H](F)C6)nc4c3F)c12] | 614.3 |
| 87 | 1-(4-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl)-1H- pyrazol-1-yl)-2- hydroxyethan-1-one | 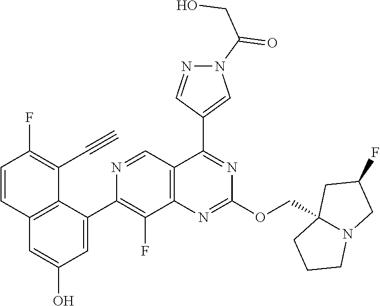 [SMILES: C#Cc1c(F)ccc2cc(O)cc(-c3ncc4c(-c5cnn(C(=O)CO)c5)nc(OC[C@@]56CCCN5C[C@H](F)C6)nc4c3F)c12] | 615.3 |
| 88 | 1-(4-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)pyrido[4,3- d]pyrimidin-4-yl)-1H- imidazol-1-yl)-2- hydroxyethan-1-one | 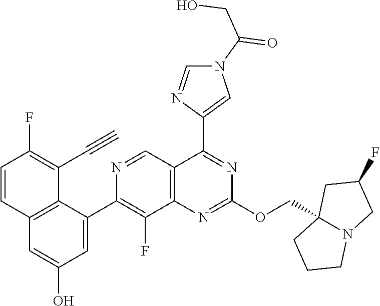 [SMILES: C#Cc1c(F)ccc2cc(O)cc(-c3ncc4c(-c5cn(C(=O)CO)cn5)nc(OC[C@@]56CCCN5C[C@H](F)C6)nc4c3F)c12] | 615.3 |

### Example 89: synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol

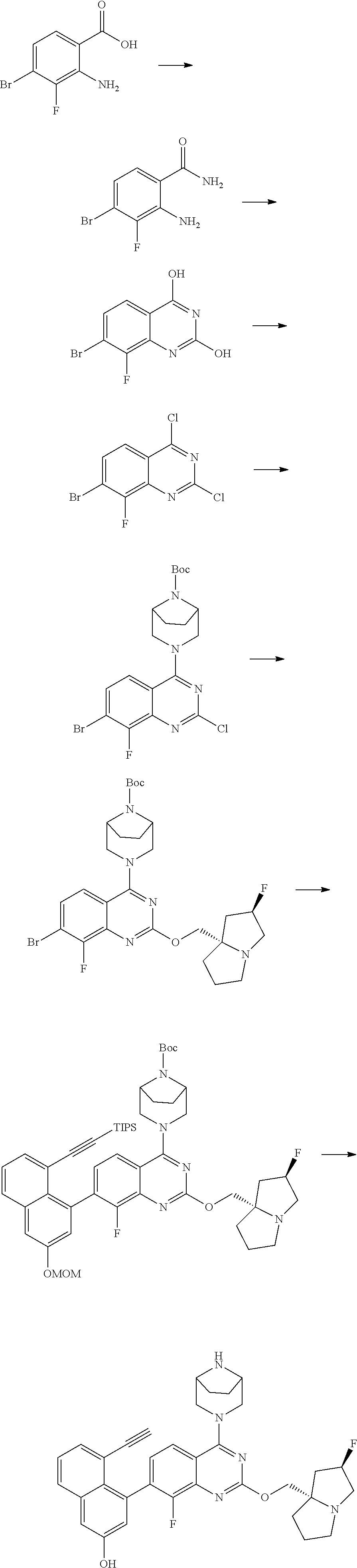

### Step 1: synthesis of 2-amino-4-bromo-3-fluorobenzamide

Dissolve 2-amino-4-bromo-3-fluorobenzoic acid (5.0 g, 20 mmol) in 50 mL DMF, add TBTU (16.0 g, 50 mmol), NH₄Cl (27.0 g, 50 mmol) and DIEA (14 mL, 80 mmol) at room temperature in one portion. The reaction was stirred at room temperature for 3 hours. After completion, add 300 mL of water into the reaction. Much solid precipitated. When there is no more solid precipitated, collect the light yellow solid by filtering and use the product directly in next step (3.7 g, yield: 74%). 1HNMR (400 MHz, DMSO) δ 7.92 (s, 2H), 7.66 (s, 1H), 6.94 (s, 1H), 6.25 (s, 2H).

### Step 2: synthesis of 7-bromo-8-fluoroquinazoline-2,4-diol

Dissolve 2-amino-4-bromo-3-fluorobenzamide (3.6 g, 14.4 mmol) in 40 mL of DMF, add CDI (9.3 g, 57.7 mmol) and K₂CO₃ (8.0 g, 50 mmol) in one portion at room temperature. The reaction was heated at 80° C. and stirred overnight. After completion, add 300 mL of water into the reaction. Much solid precipitated. When there is no more solid precipitating, collect the light yellow solid by filtering and use the product directly in next step (3.9 g, yield: 90%). 1HNMR (400 MHz, DMSO) δ 11.34 (s, 1H), 11.40 (s, 1H), 8.12 (s, 1H), 7.93 (s, 1H).

### Step 3: synthesis of 7-bromo-2,4-dichloro-8-fluoroquinazoline

Dissolve 7-bromo-8-fluoroquinazoline-2,4-diol (3.9 g, 14 mmol) in 50 mL of POCl3, add 5 mL of N,N-diethyl aniline at room temperature. The reaction was heated at 110° C. and stirred overnight. After completion, solvent was removed under vacuum to give crude product. The crude product was purified with column chromatography (PE) to give yellow solid (2.62 g, 60%). 1HNMR (400 MHz, CDCl3) δ 8.12 (s, 1H), 7.93 (s, 1H).

### Step 4: synthesis of tert-butyl (1R,5S)-3-(7-bromo-2-chloro-8-fluoroquinazolin-4-yl)-3,8-diazabicyclo[3.2.1]octane-8-carboxylate

7-bromo-2,4-dichloro-8-fluoroquinazoline (1.0 g, 3.38 mmol) and tert-butyl (1R,5S)-3,8-diazabicyclo[3.2.1]octane-8-carboxylate (789 mg, 3.72 mmol) were added to anhydrous DMF (10 mL). DIEA (655 mg, 0.83 mL, 5.07 mmol) was added with ice-water bath. The reaction was stirred in ice-water bath for 30 min. After completion, water was added and ethyl acetate was used to extract the reaction. The organic layer was washed with brine twice, dried over sodium sulfate and concentrated to give light yellow solid. Washing with PE, filtering and dried in vacuum gave light yellow solid (1.5 g, yield: 94%). 1H NMR (400 MHz, CDCl3) δ 7.57-7.42 (m, 2H), 4.37 (s, 4H), 3.68 (d, J=71.0 Hz, 2H), 1.99-1.88 (m, 2H), 1.73 (s, 2H), 1.52 (s, 9H).

### Step 5: synthesis of tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

((2R,7aS)-2-fluorotetrahydro-1H-pyrrolizin-7a(5H)-yl)methanol (220 mg, 1.38 mmol) was dissolved in anhydrous THF (5 mL). With cooling by ice-water bath, 60% NaH (51 mg, 1.27 mmol) was added, the reaction was stirred in ice-water bath for 20 min. tert-butyl (1R,5S)-3-(7-bromo-2-chloro-8-fluoroquinazolin-4-yl)-3,8-diazabicyclo[3.2.1]octane-8-carboxylate (500 mg, 1.06 mmol) was added. The stirring was kept for 4 more hours. After completion, the reaction was quenched by adding cooled water and extracted with ethyl acetate. The organic layer was washed with brine, dried over sodium sulfate and concentrated. Purification with column chromatography gave off-white solid (260 mg, yield: 41%). 1H NMR (400 MHz, CDCl3) δ 7.41 (d, J=9.1 Hz, 1H), 7.32-7.27 (m, 1H), 5.28 (d, J=52 Hz, 1H), 4.33 (s, 4H), 4.24 (d, J=10.3 Hz, 1H), 4.12 (d, J=10.3 Hz, 1H), 3.54 (s, 2H), 3.20 (d, J=28.1 Hz, 3H), 3.03-2.93 (m, 1H), 2.33-2.11 (m, 3H), 1.76 (d, J=7.2 Hz, 2H), 1.63 (s, 2H), 1.51 (s, 9H).

### Step 6: synthesis of tert-butyl (1R,5S)-3-(8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3,8-diazabicyclo[3.2.1]octane-8-carboxylate

tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60 mg, 0.10 mmol), triisopropyl((6-(methoxymethoxy)-8-(4,4,5,5-tetramethyl-1,3,2-dioxaborolan-2-yl)naphthalen-1-yl)ethynyl)silane (65 mg, 0.13 mmol), cesium carbonate (82 mg, 0.25 mmol) and Pd(dppf)Cl₂ (8 mg, 0.01 mmol) were added to 1,4-dioxane/water=5/1 (3 mL). Run evacuating and backfilling with nitrogen, heat the reaction at 100° C. for 12 hours. After completion, cooled the reaction to room temperature, add water and extract the reaction with ethyl acetate. The organic layer was dried over sodium sulfate and concentrated. Purification with TLC gave off-white solid (20 mg, yield: 23%). 1H NMR (400 MHz, CDCl3) δ 7.80 (d, J=8.2 Hz, 1H), 7.65 (d, J=7.1 Hz, 1H), 7.58 (d, J=8.9 Hz, 1H), 7.48 (s, 1H), 7.41 (d, J=7.9 Hz, 1H), 7.24-7.19 (m, 1H), 7.15 (d, J=2.6 Hz, 1H), 5.31 (s, 3H), 4.36 (s, 4H), 4.06 (s, 2H), 3.52 (s, 3H), 3.31-3.17 (m, 2H), 3.05-2.90 (m, 1H), 2.23 (s, 3H), 1.94 (d, J=31.1 Hz, 8H), 1.51 (d, J=3.3 Hz, 9H), 1.25 (d, J=5.3 Hz, 21H).

### Step 7: synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol

tert-butyl (1R,5S)-3-(8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3,8-diazabicyclo[3.2.1]octane-8-carboxylate (20 mg, 0.02 mmol) was dissolved in DMF (3 mL). CsF (17 mg, 0.11 mmol) was added and the mixture was stirred at room temperature for 2 hours. After completion, water was added and the mixture was extracted with ethyl acetate. The organic layer was washed with brine, dried over sodium sulfate and concentrated. The crude product was dissolved in acetonitrile (0.5 mL), 4M HCl in 1,4-dioxane (0.5 mL) was added. The mixture was stirred at room temperature for 30 min. After completion, saturated aqueous sodium bicarbonate was carefully added to adjust pH to 7. The mixture was extracted with DCM, the organic layer was dried over sodium sulfate and concentrated. Purification with prep-TLC (DCM/MeOH (7N NH₃)=10/1) gave light yellow solid (8 mg, yield: 61%). 1H NMR (400 MHz, CDCl3) δ 7.75 (d, J=8.2 Hz, 1H), 7.52 (dd, J=12.8, 6.0 Hz, 2H), 7.35-7.24 (m, 3H), 7.19-7.08 (m, 2H), 5.37 (t, J=29.1 Hz, 1H), 4.46 (s, 2H), 4.35 (s, 1H), 3.81 (s, 2H), 3.76-3.65 (m, 4H), 3.10 (dd, J=14.7, 7.4 Hz, 2H), 2.45-2.17 (m, 11H). MS m/z: 582.6 [M+H]+

### Example 90: synthesis of 5-ethynyl-6-fluoro-4-(8-fluoro-2-(((2R,7aS)-2-fluorotetrahydro-1H-pyrrolizin-7a(5H)-yl)methoxy)-4-(1,8-diazaspiro[4,5]decan-8-yl)quinazolin-7-yl)naphthalen-2-ol

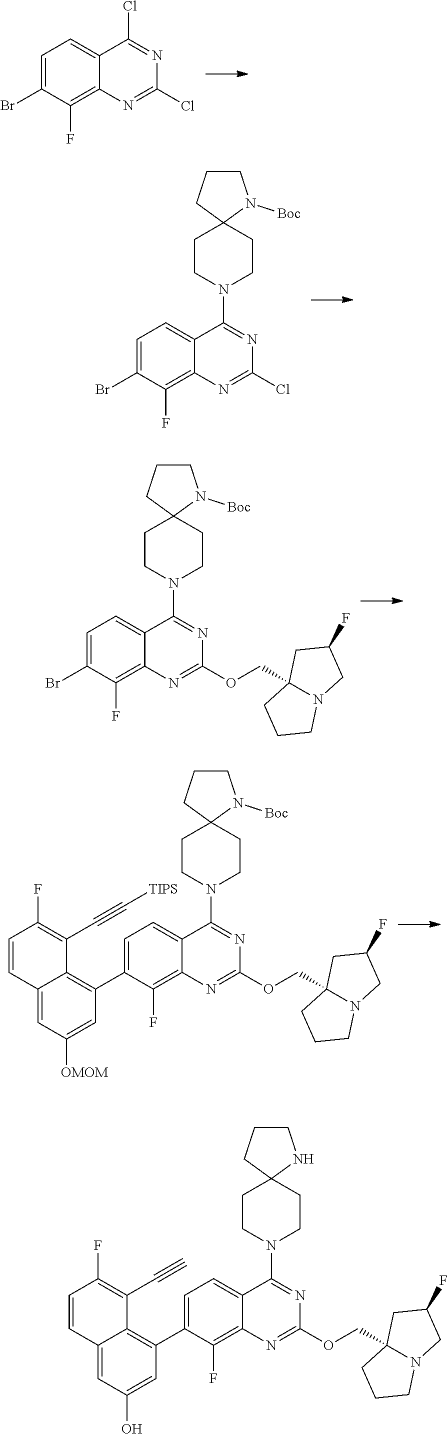

### Step 1: synthesis of tert-butyl 8-(7-bromo-2-chloro-8-fluoroquinazolin-4-yl)-1,8-diazaspiro[4.5]decane-1-carboxylate

7-bromo-2,4-dichloro-8-fluoroquinazoline (150 mg, 0.51 mmol) [symbol] tert-butyl 1,8-diazaspiro[4.5]decane-1-carboxylate (128 mg, 0.53 mmol) was dissolved in DMF (3 mL). With cooling by ice-water bath, DIEA (98 mg, 0.76 mmol) was added. The reaction was stirred in ice-water bath for 15 min. After completion, water was added. The mixture was extracted with ethyl acetate. The organic layer was washed with brine twice, dried over sodium sulfate and concentrated to give light yellow solid. Solid was washed with PE and dried in vacuo to give light yellow solid (240 mg, yield: 95%). 1H NMR (400 MHz, CDCl3) δ 7.64-7.42 (m, 2H), 4.44 (s, 2H), 3.47 (s, 2H), 3.26 (t, J=13.0 Hz, 2H), 2.98 (d, J=13.7 Hz, 1H), 2.76 (s, 1H), 2.04 (d, J=29.8 Hz, 2H), 1.84 (dd, J=13.6, 6.8 Hz, 2H), 1.55-1.53 (m, 2H), 1.48-1.34 (m, 9H).

### Step 2: synthesis of tert-butyl 8-(7-bromo-8-fluoro-2-(((2R,7aS)-2-fluorotetrahydro-1H-pyrrolizin-7a(5H)-yl)methoxy)quinazolin-4-yl)-1,8-diazaspiro[4.5]decane-1-carboxylate

((2R,7aS)-2-fluorotetrahydro-1H-pyrrolizin-7a(5H)-yl)methanol (84 mg, 0.53 mmol) was dissolved in anhydrous THF (5 mL). With ice-water bath cooling, 60% NaH (21 mg, 0.53 mmol) was added. The reaction was stirred in ice-water bath for 20 min. tert-butyl 8-(7-bromo-2-chloro-8-fluoroquinazolin-4-yl)-1,8-diazaspiro[4.5]decane-1-carboxylate (240 mg, 0.48 mmol) was added and the stirring was kept for 4 hours more. After completion, the reaction was quenched with water and extracted with ethyl acetate. The organic layer was dried over sodium sulfate and concentrated. Purification with column chromatography gave off-white solid (160 mg, yield: 54%). 1H NMR (400 MHz, MeOD) δ 7.68 (d, J=8.0 Hz, 1H), 7.47-7.43 (m, 1H), 5.33 (d, J=54.0 Hz, 1H), 4.53-4.47 (m, 2H), 4.34-4.19 (m, 2H), 3.46 (t, J=6.7 Hz, 2H), 3.39-3.31 (m, 4H), 3.08-3.04 (m, 1H), 2.93-2.86 (m, 1H), 2.77-2.72 (m, 1H), 2.45-1.78 (m, 11H), 1.53 (d, J=13.0 Hz, 2H), 1.39 (d, J=33.8 Hz, 9H).

### Step 3: synthesis of tert-butyl 8-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-1,8-diazaspiro[4.5]decane-1-carboxylate

tert-butyl 8-(7-bromo-8-fluoro-2-(((2R,7aS)-2-fluorotetrahydro-1H-pyrrolizin-7a(5H)-yl)methoxy)quinazolin-4-yl)-1,8-diazaspiro[4.5]decane-1-carboxylate (50 mg, 0.08 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (50 mg, 0.10 mmol), cesium carbonate (50 mg, 0.15 mmol) and Pd(dppf)Cl₂ (9 mg, 0.01 mmol) was added to 1,4-dioxane/water=5/1(3 mL). Run evacuating and backfilling with nitrogen, the reaction was stirred at 100° C. in nitrogen atmosphere for 12 hours. After completion, the reaction was cooled to room temperature. Water was added and the reaction was extracted with ethyl acetate. The organic layer was dried over sodium sulfate and concentrated. Purification with prep-TLC gave off-white solid (55 mg, yield: 59%). MS m/z: 928 [M+H]+

### Step 4: synthesis of 5-ethynyl-6-fluoro-4-(8-fluoro-2-(((2R,7aS)-2-fluorotetrahydro-1H-pyrrolizin-7a(5H)-yl)methoxy)-4-(1,8-diazaspiro[4.5]decan-8-yl)quinazolin-7-yl)naphthalen-2-ol

tert-butyl 8-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-1,8-diazaspiro[4.5]decane-1-carboxylate (50 mg, 0.05 mmol) was dissolve in DMF (3 mL). CsF (41 mg, 0.27 mmol) was added and the reaction was stirred at room temperature for 2 hours. After completion, water was added and the mixture was extracted with ethyl acetate. The organic layer was washed with sodium sulfate and concentrated. The crude product was dissolved in acetonitrile (2 mL). 4M HCl in 1,4-dioxane (1 mL) was added, the mixture was stirred at room temperature for 30 min. After completion, saturated aqueous sodium bicarbonate was added carefully to adjust pH to 7. The mixture was extracted with DCM. The organic layer was dried over sodium sulfate and concentrated. Pre-TLC (DCM/MeOH(7N NH₃)=10/1) gave light yellow solid (25 mg, yield: 74%). MS m/z: 628.64 [M+H]+

The compounds of Examples 91-156 were prepared by the preparation method for Example 89

| Ex. | Compound name | Structural formula | m/z: ES⁺[M + H] |
|---|---|---|---|
| 91 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)- yl)methoxy)quinazolin- 7-yl)-5-ethynyl-6- fluoronaphthalen-2-ol | 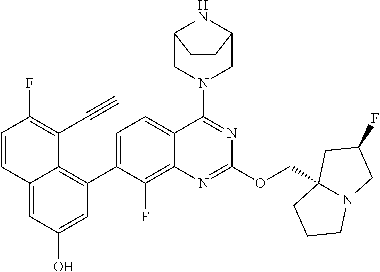 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | ¹H NMR (400 MHZ, MeOD) δ 7.85 (dd, J = 8.4, 4.6 Hz, 2H), 7.31 (d, J = 2.6 Hz, 3H), 7.09 (s, 1H), 5.48 (d, J = 53.2 Hz, 1H), 4.75- 4.46 (m, 4H), 4.05 (s, 2H), 3.90-3.63 (m, 5H), 2.79 (s, 1H), 2.67-2.43 (m, 2H), 2.38- 2.20 (m, 4H), 2.07 (s, 5H). MS m/z: 600.62 |
| 92 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)-yl) methoxy)quinazolin- 7-yl)-5-ethyl-6- fluoronaphthalen-2-ol | 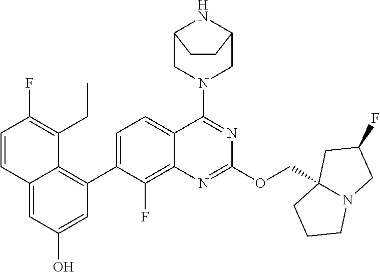 [SMILES: CCc1c(F)ccc2cc(O)cc(-c3ccc4c(N5CC6CCC(C5)N6)nc(OC[C@@]56CCCN5C[C@H](F)C6)nc4c3F)c12] | 604.28 |
| 93 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)-yl) methoxy)quinazolin- 7-yl)-5- ethylnaphthalen-2-ol | 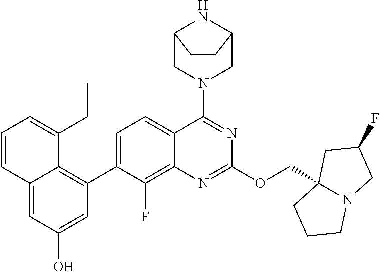 [SMILES: CCc1cccc2cc(O)cc(-c3ccc4c(N5CC6CCC(C5)N6)nc(OC[C@@]56CCCN5C[C@H](F)C6)nc4c3F)c12] | 586 |
| 94 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)-yl) methoxy)quinazolin- 7-yl)-5,6- difluoronaphthalen- 2-ol | 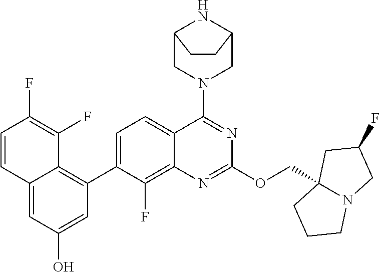 [SMILES: Oc1cc(-c2ccc3c(N4CC5CCC(C4)N5)nc(OC[C@@]45CCCN4C[C@H](F)C5)nc3c2F)c2c(F)c(F)ccc2c1] | 594.24 |
| 95 | 4-(4-((1R,5S)-3,8- diazabicyclo[3.2.1] octan-3-yl)-8-fluoro- 2-(((2R,7aS)-2- fluorotetrahydro-1H- pyrrolizin-7a(5H)-yl) methoxy)quinazolin- 7-yl)-5- fluoronaphthalen-2- ol | 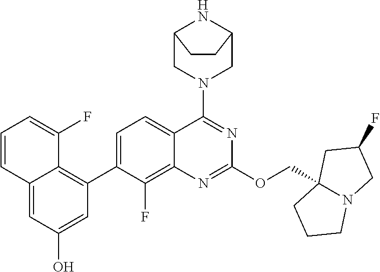 [SMILES: Oc1cc(-c2ccc3c(N4CC5CCC(C4)N5)nc(OC[C@@]45CCCN4C[C@H](F)C5)nc3c2F)c2c(F)cccc2c1] | 576.25 |
| 96 | ((3R,7aR)-7a-(((4- ((1R,5S)-3,8- diazabicyclo[3.2.1] octan-3-yl)-7-(8- ethynyl-7-fluoro-3- hydroxynaphthalen- 1-yl)-8- fluoroquinazolin-2- yl)oxy)methyl) hexahydro-1H- pyrrolizin-3- yl)methyl dimethylcarbamate | 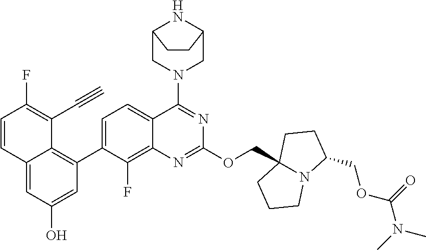 [SMILES: C#Cc1c(F)ccc2cc(O)cc(-c3ccc4c(N5CC6CCC(C5)N6)nc(OC[C@]56CCCN5[C@@H](COC(=O)N(C)C)CC6)nc4c3F)c12] | 683.31 |
| 97 | ((3R,7aR)-7a-(((4- ((1R,5S)-3,8- diazabicyclo[3.2.1] octan-3-yl)-7-(8- ethyl-7-fluoro-3- hydroxynaphthalen- 1-yl)-8- fluoroquinazolin-2- yl)oxy)methyl) hexahydro-1H- pyrrolizin-3-yl) methyl dimethylcarbamate | 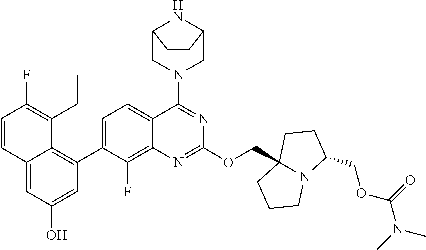 [SMILES: CCc1c(F)ccc2cc(O)cc(-c3ccc4c(N5CC6CCC(C5)N6)nc(OC[C@]56CCCN5[C@@H](COC(=O)N(C)C)CC6)nc4c3F)c12] | 687.34 |
| 98 | ((3R,7aR)-7a-(((4- ((1R,5S)-3,8- diazabicyclo[3.2.1] octan-3-yl)-7-(8- ethynyl-7- fluoronaphthalen-1- yl)-8- fluoroquinazolin-2- yl)oxy)methyl) hexahydro-1H- pyrrolizin-3-yl) methyl dimethylcarbamate | 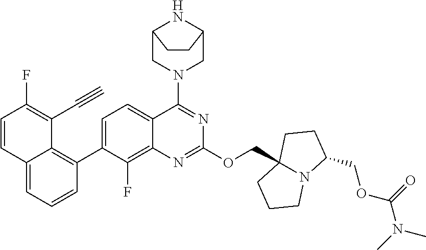 [SMILES: C#Cc1c(F)ccc2cccc(-c3ccc4c(N5CC6CCC(C5)N6)nc(OC[C@]56CCCN5[C@@H](COC(=O)N(C)C)CC6)nc4c3F)c12] | 667.31 |
| 99 | 4-(4-((1R,5S)-3,8- diazabicyclo[3.2.1] octan-3-yl)-8-fluoro- 2-((1-(pyrrolidin-1- ylmethyl)cyclopropyl) methoxy)quinazolin- 7-yl)-5-ethynyl-6- fluoronaphthalen-2-ol | 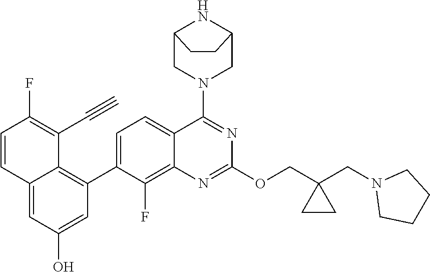 [SMILES: C#Cc1c(F)ccc2cc(O)cc(-c3ccc4c(N5CC6CCC(C5)N6)nc(OCC5(CN6CCCC6)CC5)nc4c3F)c12] | 596.28 |
| 100 | 4-(4-((1R,5S)-3,8- diazabicyclo[3.2.1] octan-3-yl)-8-fluoro- 2-((1-(pyrrolidin-1- ylmethyl)cyclopropyl) methoxy)quinazolin- 7-yl)-5-ethyl-6- fluoronaphthalen-2-ol | 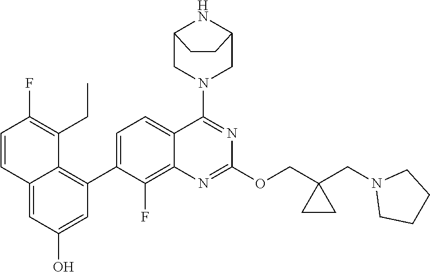 [SMILES: CCc1c(F)ccc2cc(O)cc(-c3ccc4c(N5CC6CCC(C5)N6)nc(OCC5(CN6CCCC6)CC5)nc4c3F)c12] | 600.31 |
| 101 | 4-(4-((1R,5S)-3,8- diazabicyclo[3.2.1] octan-3-yl)-8-fluoro- 2-((1-(pyrrolidin-1- ylmethyl)cyclopropyl) methoxy)quinazolin- 7-yl)-5,6- difluoronaphthalen-2- ol | 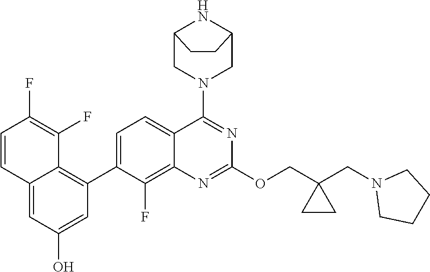 [SMILES: Oc1cc(-c2ccc3c(N4CC5CCC(C4)N5)nc(OCC4(CN5CCCC5)CC4)nc3c2F)c2c(F)c(F)ccc2c1] | 590.27 |
| 102 | 4-(4-((1R,5S)-3,8- diazabicyclo[3.2.1] octan-3-yl)-8-fluoro- 2-((1-(pyrrolidin-1- ylmethyl)cyclopropyl) methoxy)quinazolin- 7-yl)-5- fluoronaphthalen-2-ol | 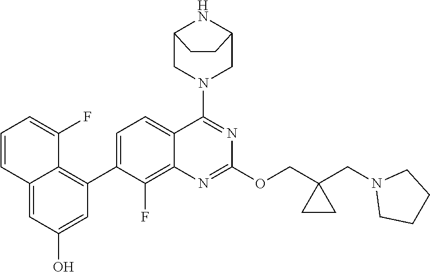 [SMILES: Oc1cc(-c2ccc3c(N4CC5CCC(C4)N5)nc(OCC4(CN5CCCC5)CC4)nc3c2F)c2c(F)cccc2c1] | 572.28 |
| 103 | 4-(4-((1R,5S)-3,8- diazabicyclo[3.2.1] octan-3-yl)-8-fluoro- 2-((1-(pyrrolidin-1- ylmethyl)cyclopropyl) methoxy)quinazolin- 7-yl)-5- ethynylnaphthalen-2-ol | 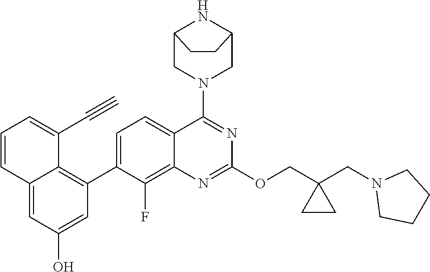 [SMILES: C#Cc1cccc2cc(O)cc(-c3ccc4c(N5CC6CCC(C5)N6)nc(OCC5(CN6CCCC6)CC5)nc4c3F)c12] | 578.29 |
| 104 | 4-(4-((1R,5S)-3,8- diazabicyclo[3.2.1] octan-3-yl)-8-fluoro-2- ((1-(pyrrolidin-1- ylmethyl)cyclopropyl) methoxy)quinazolin- 7-yl)-5-ethylnaphthalen- 2-ol | 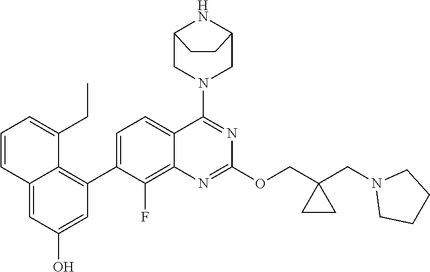 [SMILES: CCc1cccc2cc(O)cc(-c3ccc4c(N5CC6CCC(C5)N6)nc(OCC5(CN6CCCC6)CC5)nc4c3F)c12] | 582.32 |
| 105 | 4-(4-(3,9-diazabicyclo [3.3.1]nonan-3-yl)-8- fluoro-2-(((2R,7aS)-2- fluorotetrahydro- 1H-pyrrolizin-7a(5H)- yl)methoxy)quinazolin- 7-yl)-5-ethynyl-6- fluoronaphthalen-2-ol | 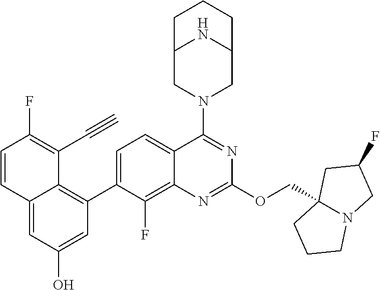 | 614.27 |
| 106 | 4-(4-(3,9-diazabicyclo [3.3.1]nonan-3-yl)-8- fluoro-2-(((2R,7aS)-2- fluorotetrahydro- 1H-pyrrolizin-7a(5H)- yl)methoxy)quinazolin- 7-yl)-5- ethynylnaphthalen-2-ol | 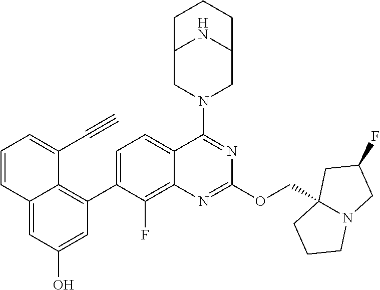 | 596 |
| 107 | 4-(4-(3,6-diazabicyclo [3.1.1 ]heptan-3-yl)-8- fluoro-2-(((2R,7aS)-2- fluorotetrahydro- 1H-pyrrolizin-7a(5H)- yl)methoxy)quinazolin- 7-yl)-5-ethynyl-6- fluoronaphthalen-2-ol | 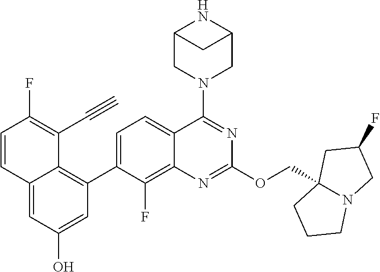 | 586.24 |
| 108 | 4-(4-(2,5-diazabicyclo [2.2.2]octan-2-yl)-8- fluoro-2-(((2R,7aS)-2- fluorotetrahydro- 1H-pyrrolizin-7a(5H)- yl)methoxy)quinazolin- 7-yl)-5-ethynyl-6- fluoronaphthalen-2-ol | 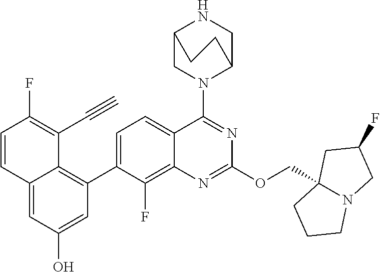 | 600.25 |
| 109 | 5-ethynyl-6-fluoro-4- (8-fluoro-2-(((2R,7aS)- 2-fluorotetrahydro-1H- pyrrolizin-7a(5H)-yl) methoxy)-4-(1-thia-8- azaspiro[4.5]decan-8- yl)quinazolin-7- yl)naphthalen-2-ol | 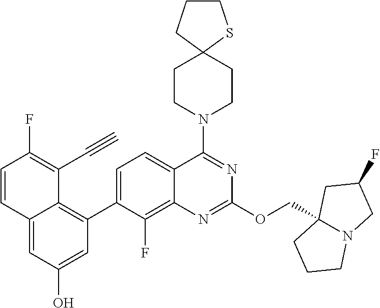 | 645.24 |
| 110 | 8-(7-(8-ethynyl-7- fluoro-3- hydroxynaphthalen-1- yl)-8-fluoro-2-(((2R, 7aS)-2- fluorotetrahydro-1H- pyrrolizin-7a(5H)-yl) methoxy)quinazolin- 4-yl)-1-thia-8-azaspiro [4.5]decane 1,1- dioxide | 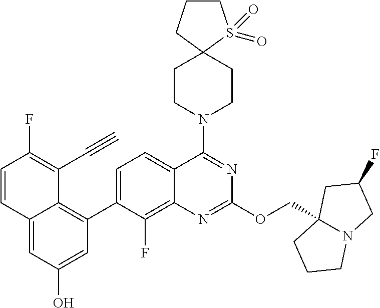 | 677.23 |
| 111 | 4-(4-(3- aminopyrrolidin-1-yl)- 8-fluoro-2-(((2R,7aS)- 2-fluorotetrahydro-1H- pyrrolizin-7a(5H)-yl) methoxy)quinazolin- 7-yl)-5-ethynyl-6- fluoronaphthalen-2-ol | 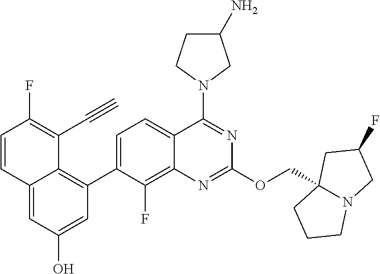 | 574.24 |
| 112 | 5-ethynyl-6-fluoro-4- (8-fluoro-2-(((2R,7aS)- 2-fluorotetrahydro-1H- pyrrolizin-7a(5H)-yl) methoxy)-4- (octahydro- 6H-pyrrolo[3,4-b] pyridin-6-yl) quinazolin-7-yl) naphthalen-2-ol | 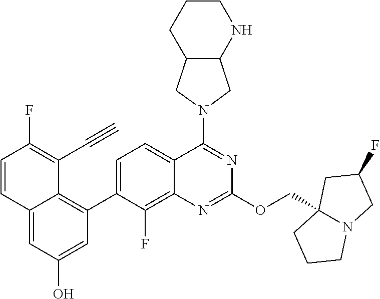 | 614.27 |
| 113 | 5-ethynyl-6-fluoro-4- (8-fluoro-2-(((2R,7aS)- 2-fluorotetrahydro-1H- pyrrolizin-7a(5H)-yl) methoxy)-4- (hexahydropyrrolo[3,4- b]pyrrol-5(1H)-yl) quinazolin-7-yl) naphthalen-2-ol | 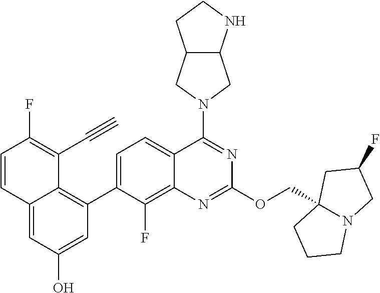 | 600.25 |
| 114 | 4-(4-((3S,4S)-4-amino- 3-methyl-2-oxa-8- azaspiro[4.5]decan-8- yl)-8-fluoro-2-(((2R, 7aS)-2-fluorotetrahydro- 1H-pyrrolizin-7a(5H)- yl)methoxy)quinazolin- 7-yl)-5-ethynyl-6- fluoronaphthalen-2-ol | 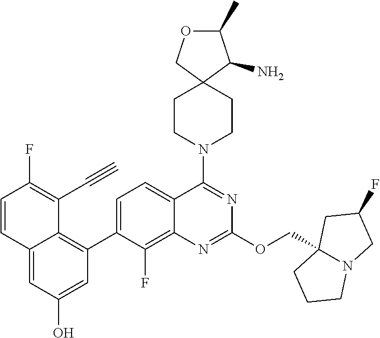 | 658.29 |
| 115 | 4-(4-((3S,4S)-4-amino- 3-methyl-2-oxa-8- azaspiro[4.5]decan-8- yl)-8-fluoro-2-(((2R, 7aS)-2-fluorotetrahydro- 1H-pyrrolizin-7a(5H)- yl)methoxy)quinazolin- 7-yl)-5- ethynylnaphthalen-2-ol | 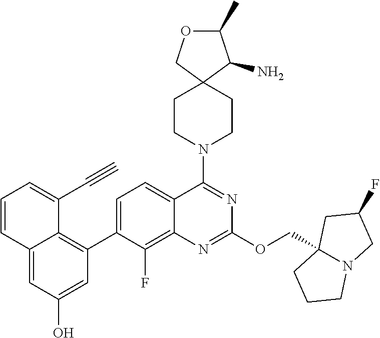 | 640.3 |
| 116 | 4-(4-(3-aminopyrrolidin- 1-yl)-8-fluoro-2-(((2R, 7aS)-2-fluorotetrahydro- 1H-pyrrolizin-7a(5H)- yl)methoxy)quinazolin- 7-yl)-5-ethynyl-6- fluoronaphthalen-2-ol | 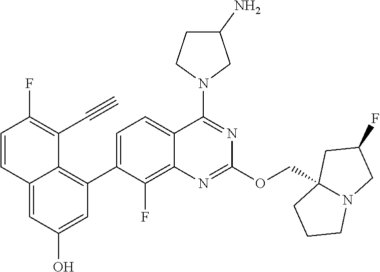 | 574.24 |
| 117 | N-(1-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)quinazolin- 4-yl)pyrrolidin-3- yl)methanesulfonamide | 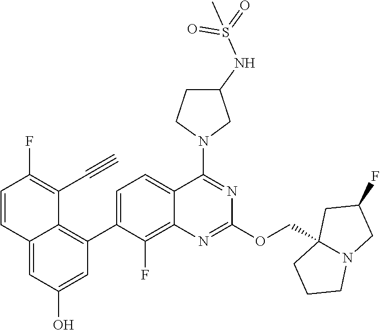 | 652.21 |
| 118 | (1R,5S,8R)-3-(7-(8- ethynyl-7-fluoro-3- hydroxynaphthalen- 1-yl)-8-fluoro-2- (((2R,7aS)-2- fluorotetrahydro-1H- pyrrolizin-7a(5H)- yl)methoxy)quinazolin- 4-yl)-3-azabicyclo [3.2.1]octan-8-ol | 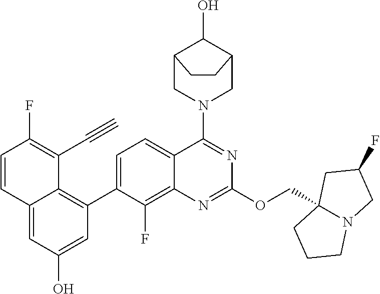 | 615.25 |
| 119 | 4-(4-((1R,5S)-8,8- difluoro-3-azabicyclo [3.2.1]octan-3-yl)-8- fluoro-2-(((2R,7aS)- 2-fluorotetrahydro- 1H-pyrrolizin-7a(5H)- yl)methoxy)quinazolin- 7-yl)-5-ethynyl-6- fluoronaphthalen-2-ol | 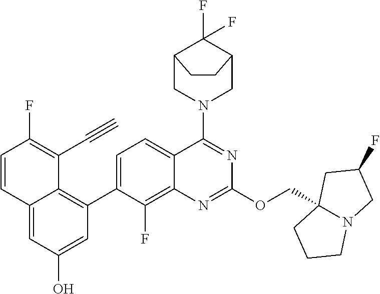 | 635.64 |
| 120 | 4-(4-((1R,5S)-8,8- difluoro-3-azabicyclo [3.2.1]octan-3-yl)-8- fluoro-2-(((2R,7aS)- 2-fluorotetrahydro- 1H-pyrrolizin-7a(5H)- yl)methoxy)quinazolin- 7-yl)-5-ethyl-6- fluoronaphthalen-2-ol | 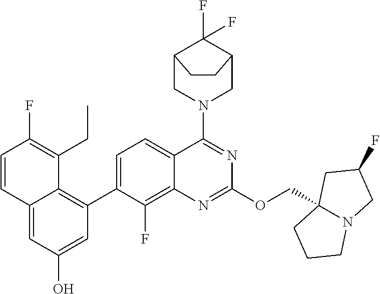 | 639 |
| 121 | 4-(4-((1R,5S)-8,8- difluoro-3-azabicyclo [3.2.1]octan-3-yl)-8- fluoro-2-(((2R,7aS)- 2-fluorotetrahydro- 1H-pyrrolizin-7a(5H)- yl)methoxy)quinazolin- 7-yl)-5- ethynylnaphthalen-2-ol | 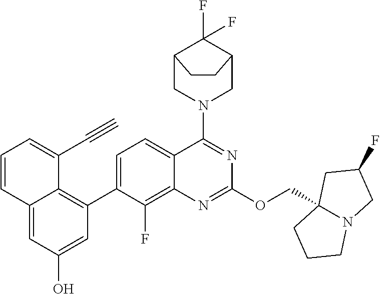 | 617 |
| 122 | 4-(4-((1R,5S)-8,8- difluoro-3-azabicyclo [3.2.1]octan-3-yl)-8- fluoro-2-(((2R,7aS)- 2-fluorotetrahydro- 1H-pyrrolizin-7a(5H)- yl)methoxy)quinazolin- 7-yl)-5,6- difluoronaphthalen-2-ol | 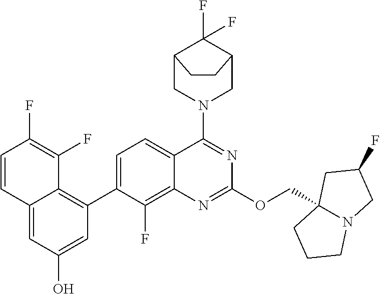 | 629 |
| 123 | 5-ethynyl-6-fluoro-4- (8-fluoro-4-((1R,5S, 8R)-8-fluoro-3- azabicyclo[3.2.1]octan- 3-yl)-2-(((2R,7aS)- 2-fluorotetrahydro-1H- pyrrolizin-7a(5H)-yl) methoxy)quinazolin-7- yl)naphthalen-2-ol | 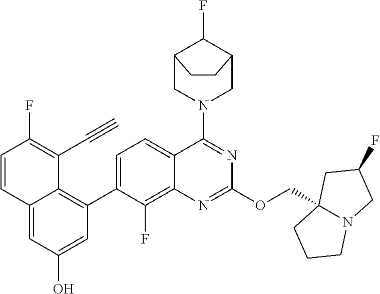 | 617 |
| 124 | 5-ethyl-6-fluoro-4-(8- fluoro-4-((1R,5S, 8R)-8-fluoro-3- azabicyclo[3.2.1]octan- 3-yl)-2-(((2R,7aS)-2- fluorotetrahydro-1H- pyrrolizin-7a(5H)- yl)methoxy)quinazolin- 7-yl)naphthalen-2-ol | 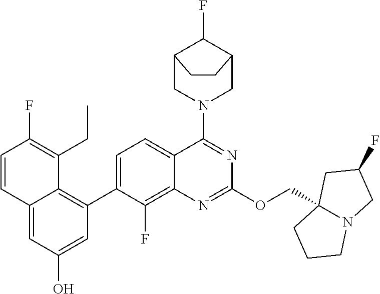 | 621 |
| 125 | 5-ethynyl-4-(8-fluoro- 4-((1R,5S,8R)-8- fluoro-3-azabicyclo [3.2.1]octan-3-yl)-2- (((2R,7aS)-2- fluorotetrahydro-1H- pyrrolizin-7a(5H)-yl) methoxy)quinazolin- 7-yl)naphthalen-2-ol | 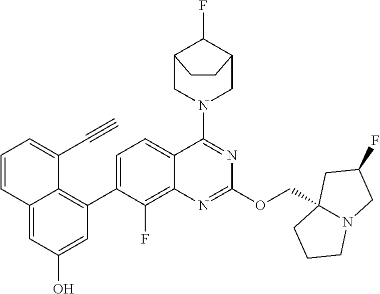 | 599 |
| 126 | 5,6-difluoro-4-(8- fluoro-4-((1R,5S,8R)- 8-fluoro-3-azabicyclo [3.2.1]octan-3-yl)-2- (((2R,7aS)-2- fluorotetrahydro-1H- pyrrolizin-7a(5H)-yl) methoxy)quinazolin- 7-yl)naphthalen-2-ol | 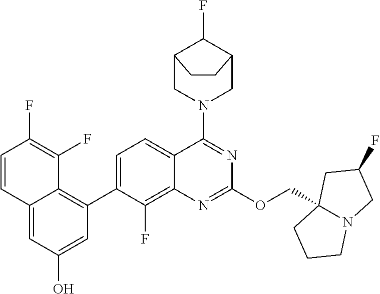 | 611 |
| 127 | (1R,5S,8S)-3-(7-(8- ethynyl-7-fluoro-3- hydroxynaphthalen- 1-yl)-8-fluoro-2- (((2R,7aS)-2- fluorotetrahydro-1H- pyrrolizin-7a(5H)-yl) methoxy)quinazolin- 4-yl)-3-azabicyclo [3.2.1 ]octane-8- carbonitrile | 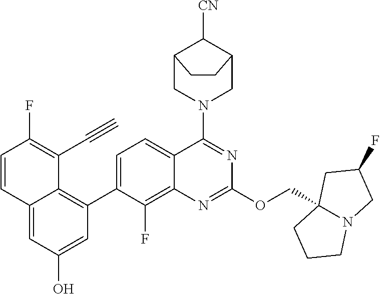 | 624.25 |
| 128 | 4-(4-((1R,5S,8R)-8- amino-3-azabicyclo [3.2.1]octan-3-yl)-8- fluoro-2-(((2R,7aS)- 2-fluorotetrahydro- 1H-pyrrolizin-7a(5H)- yl)methoxy)quinazolin- 7-yl)-5-ethynyl-6- fluoronaphthalen-2-ol | 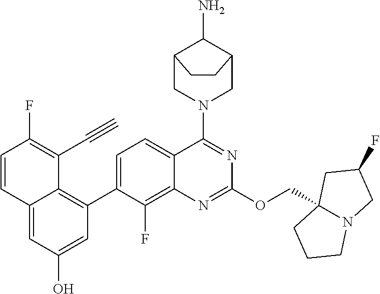 | 614.6 |
| 129 | 5-ethynyl-6-fluoro-4- (8-fluoro-2-(((2R,7aS)- 2-fluorotetrahydro- 1H-pyrrolizin-7a(5H)- yl)methoxy)-4-((1R, 5S,8R)-8-(methylthio)- 3-azabicyclo[3.2.1] octan-3-yl)quinazolin- 7-yl)naphthalen-2-ol | 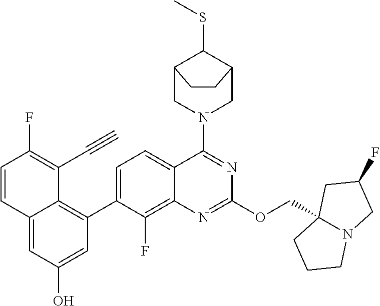 | 645.24 |
| 130 | 5-ethynyl-6-fluoro-4- (8-fluoro-2-(((2R,7aS)- 2-fluorotetrahydro-1H- pyrrolizin-7a(5H)- yl)methoxy)-4-((1R,5S, 8R)-8-(methylsulfinyl)- 3-azabicyclo[3.2.1] octan-3-yl)quinazolin- 7-yl)naphthalen-2-ol | 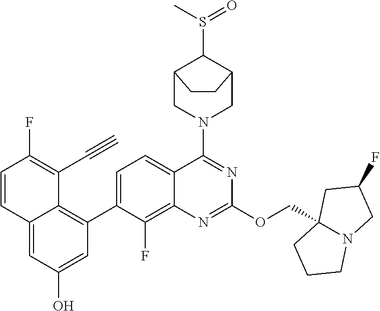 | 661.24 |
| 131 | 5-ethynyl-6-fluoro-4- (8-fluoro-2-(((2R,7aS)- 2-fluorotetrahydro-1H- pyrrolizin-7a(5H)- yl)methoxy)-4-((1R,5S, 8R)-8-(methylsulfonyl)- 3-azabicyclo[3.2.1] octan-3-yl)quinazolin- 7-yl)naphthalen-2-ol | 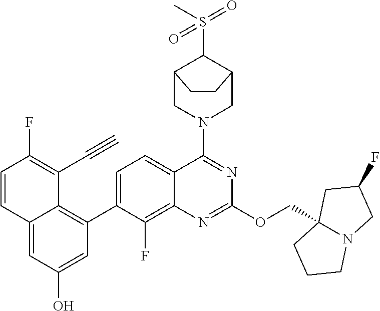 | 677.23 |
| 132 | 5-ethynyl-6-fluoro-4- (8-fluoro-2-(((2R,7aS)- 2-fluorotetrahydro-1H- pyrrolizin-7a(5H)-yl) methoxy)-4-((1R,5S, 8R)-8-((trifluoromethyl) sulfonyl)-3-azabicyclo [3.2.1]octan-3-yl) quinazolin-7-yl) naphthalen-2-ol | 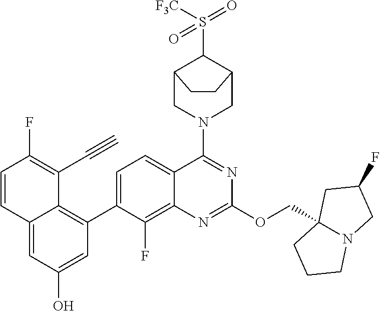 | 731.20 |
| 133 | 1-(7-(8-ethynyl-7- fluoro-3- hydroxynaphthalen-1- yl)-8-fluoro-2-(((2R, 7aS)-2-fluorotetrahydro- 1H-pyrrolizin-7a(5H)- yl)methoxy)quinazolin- 4-yl)piperidine-4- carbonitrile | 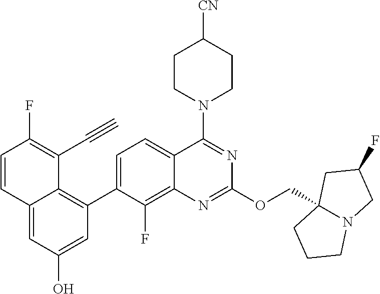 | 598.24 |
| 134 | 1-(7-(8-ethynyl-7- fluoro-3- hydroxynaphthalen-1- yl)-8-fluoro-2-(((2R, 7aS)-2- fluorotetrahydro-1H- pyrrolizin-7a(5H)-yl) methoxy)quinazolin- 4-yl)azetidine-3- carbonitrile | 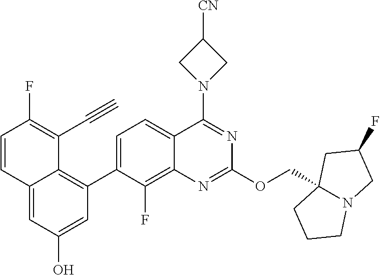 | 570.20 |
| 135 | 5-ethynyl-6-fluoro-4- (8-fluoro-2-(((2R, 7aS)-2- fluorotetrahydro-1H- pyrrolizin-7a(5H)-yl) methoxy)-4-(2,6- diazaspiro[3.3]heptan- 2-yl)quinazolin-7- yl)naphthalen-2-ol | 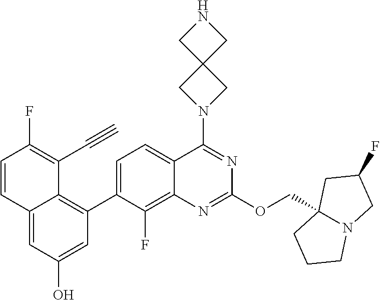 | 586.24 |
| 136 | 5-ethynyl-6-fluoro-4- (8-fluoro-2-(((2R,7aS)- 2-fluorotetrahydro-1H- pyrrolizin-7a(5H)- yl)methoxy)-4- (quinuclidin-4- yl)quinazolin-7- yl)naphthalen-2-ol | 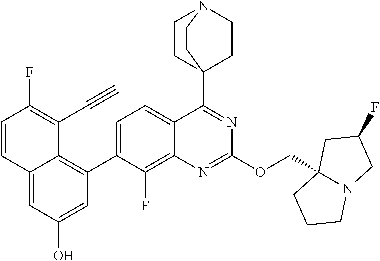 | 599.26 |
| 137 | 4-(4-((1R,5S)-8- azabicyclo[3.2.1] octan-3-yl)-8-fluoro- 2-(((2R,7aS)-2- fluorotetrahydro-1H- pyrrolizin-7a(5H)- yl)methoxy)quinazolin- 7-yl)-5-ethynyl-6- fluoronaphthalen-2-ol | 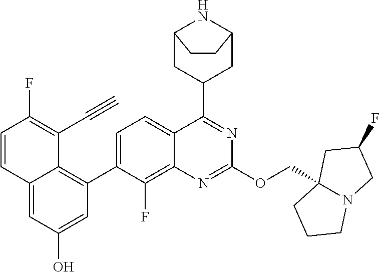 | 599.26 |
| 138 | 4-(4-((1R,5S)-8- azabicyclo[3.2.1]octan- 3-yl)-8-fluoro-2-(((2R, 7aS)-2- fluorotetrahydro-1H- pyrrolizin-7a(5H)-yl) methoxy)quinazolin-7- yl)-5- ethynylnaphthalen-2-ol | 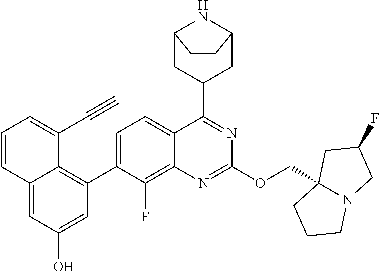 | 581 |
| 139 | 6-(4-((1R,5S)-3,8- diazabicyclo[3.2.1] octan-3-yl)-8-fluoro-2- (((2R,7aS)-2- fluorotetrahydro-1H- pyrrolizin-7a(5H)-yl) methoxy)quinazolin- 7-yl)-5-cyclopropyl-4- methylpyridin-2-amine | 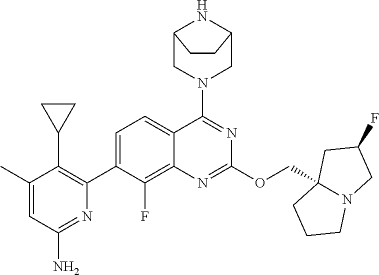 | 562.30 |
| 140 | 4-(4-((1R,5S)-3,8- diazabicyclo[3.2.1] octan-3-yl)-2-(((2R, 7aS)-2-fluorotetrahydro- 1H-pyrrolizin-7a(5H)- yl)methoxy)pyrido[2,3- d]pyrimidin-7-yl)-5- ethynyl-6- fluoronaphthalen-2-ol | 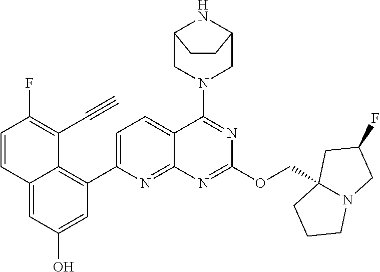 | 583.26 |
| 141 | 4-(4-((1R,5S)-3,8- diazabicyclo[3.2.1] octan-3-yl)-2-(((2R,7aS)- 2-fluorotetrahydro-1H- pyrrolizin-7a(5H)- yl)methoxy)pyrido[2,3- d]pyrimidin-7-yl)-5- ethyl-6-fluoronaphthalen- 2-ol | 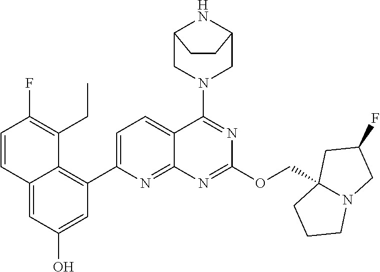 | 587.29 |
| 142 | 4-(4-((1R,5S)-3,8- diazabicyclo[3.2.1]octan- 3-yl)-2-(((2R,7aS)-2- fluorotetrahydro-1H- pyrrolizin-7a(5H)-yl) methoxy)pyrido[2,3- d]pyrimidin-7-yl)-5,6- difluoronaphthalen-2-ol | 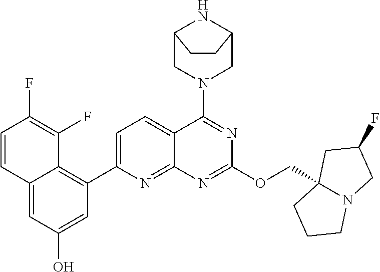 | 577.25 |
| 143 | 4-(4-((1R,5S)-3,8- diazabicyclo[3.2.1]octan- 3-yl)-2-(((2R,7aS)-2- fluorotetrahydro-1H- pyrrolizin-7a(5H)-yl) methoxy)pyrido[2,3- d]pyrimidin-7-yl)-5- fluoronaphthalen-2-ol | 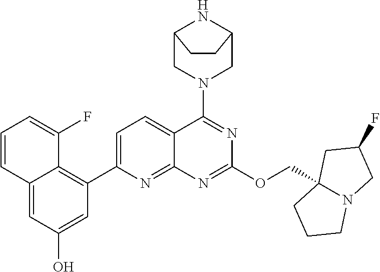 | 559.26 |
| 144 | 4-(4-((1R,5S)-3,8- diazabicyclo[3.2.1]octan- 3-yl)-2-(((2R,7aS)-2- fluorotetrahydro-1H- pyrrolizin-7a(5H)-yl) methoxy)pyrido[2,3- d]pyrimidin-7-yl)-5- ethynylnaphthalen-2-ol | 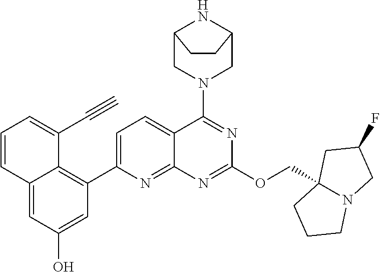 | 565.26 |
| 145 | ((3R,7aR)-7a-(((4-((1R, 5S)-3,8-diazabicyclo [3.2.1]octan-3-yl)-7-(8- ethynyl-7-fluoro-3- hydroxynaphthalen-1- yl)pyrido[2,3-d] pyrimidin-2-yl)oxy) methyl)hexahydro-1H- pyrrolizin-3-yl)methyl dimethylcarbamate | 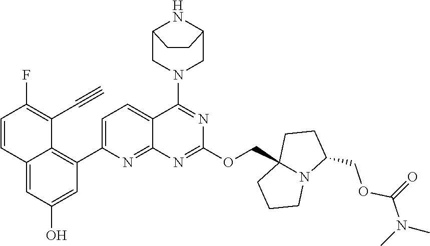 [SMILES: C#Cc1c(F)ccc2cc(O)cc(-c3ccc4c(N5CC6CCC(C5)N6)nc(OC[C@]56CCCN5[C@@H](COC(=O)N(C)C)CC6)nc4n3)c12] | 666.31 |
| 146 | ((3R,7aR)-7a-(((4-((1R, 5S)-3,8-diazabicyclo [3.2.1]octan-3-yl)-7-(8- ethyl-7-fluoro-3- hydroxynaphthalen-1- yl)pyrido[2,3-d] pyrimidin-2-yl)oxy) methyl)hexahydro-1H- pyrrolizin-3-yl)methyl dimethylcarbamate | 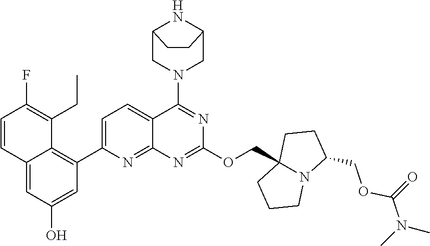 [SMILES: CCc1c(F)ccc2cc(O)cc(-c3ccc4c(N5CC6CCC(C5)N6)nc(OC[C@]56CCCN5[C@@H](COC(=O)N(C)C)CC6)nc4n3)c12] | 670.34 |
| 147 | ((3R,7aR)-7a-(((4-((1R, 5S)-3,8-diazabicyclo [3.2.1]octan-3-yl)-7-(8- ethynyl-7- fluoronaphthalen-1-yl) pyrido[2,3-d] pyrimidin-2-yl)oxy) methyl)hexahydro-1H- pyrrolizin-3-yl)methyl dimethylcarbamate | 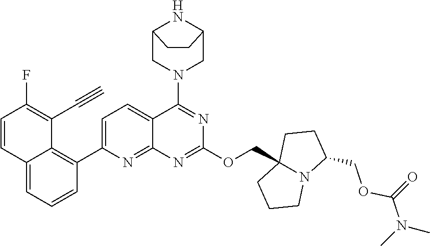 [SMILES: C#Cc1c(F)ccc2cccc(-c3ccc4c(N5CC6CCC(C5)N6)nc(OC[C@]56CCCN5[C@@H](COC(=O)N(C)C)CC6)nc4n3)c12] | 650.32 |
| 148 | 4-(4-((1R,5S)-3,8- diazabicyclo[3.2.1] octan-3-yl)-2-(((2R, 7aS)-2- fluorotetrahydro-1H- pyrrolizin-7a(5H)- yl)methoxy)quinazolin- 7-yl)-5-ethynyl-6- fluoronaphthalen-2-ol | 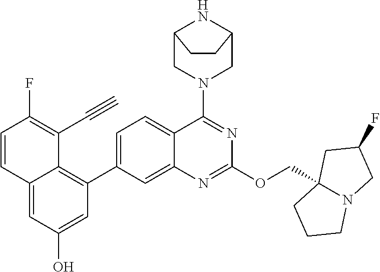 | 582.26 |
| 149 | 4-(4-((1R,5S)-3,8- diazabicyclo[3.2.1] octan-3-yl)-2-(((2R, 7aS)-2- fluorotetrahydro-1H- pyrrolizin-7a(5H)- yl)methoxy)quinazolin- 7-yl)-5-ethyl-6- fluoronaphthalen-2-ol | 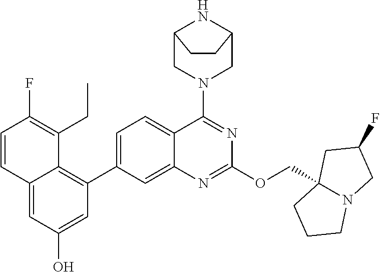 | 586.29 |
| 150 | 4-(4-((1R,5S)-3,8- diazabicyclo[3.2.1] octan-3-yl)-2-(((2R, 7aS)-2- fluorotetrahydro-1H- pyrrolizin-7a(5H)- yl)methoxy)quinazolin- 7-yl)-5,6- difluoronaphthalen-2-ol | 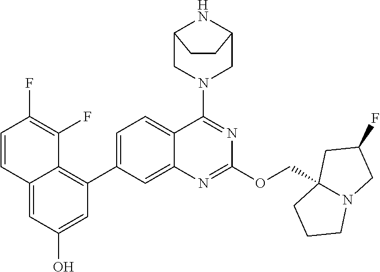 | 576.25 |
| 151 | 4-(4-((1R,5S)-3,8- diazabicyclo[3.2.1] octan-3-yl)-2-(((2R, 7aS)-2- fluorotetrahydro-1H- pyrrolizin-7a(5H)-yl) methoxy)quinazolin- 7-yl)-5- fluoronaphthalen-2-ol | 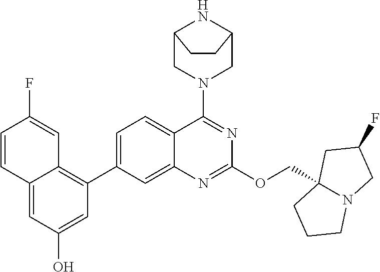 | 558.26 |
| 152 | 4-(4-((1R,5S)-3,8- diazabicyclo[3.2.1] octan-3-yl)-2-(((2R, 7aS)-2- fluorotetrahydro-1H- pyrrolizin-7a(5H)-yl) methoxy)quinazolin- 7-yl)-5- ethynylnaphthalen-2-ol | 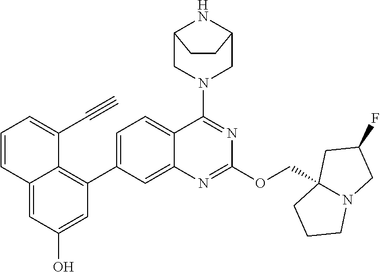 | 564.27 |
| 153 | ((3R,7aR)-7a-(((4-((1R, 5S)-3,8-diazabicyclo [3.2.1]octan-3-yl)-7-(8- ethynyl-7-fluoro-3- hydroxynaphthalen-1- yl)quinazolin-2-yl) oxy)methyl)hexahydro- 1H-pyrrolizin-3-yl) methyl dimethylcarbamate | 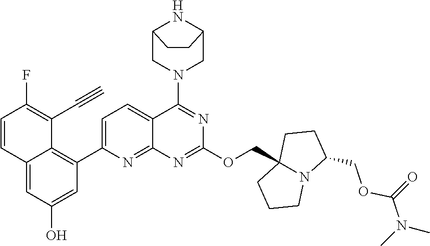 [SMILES: C#Cc1c(F)ccc2cc(O)cc(-c3ccc4c(N5CC6CCC(C5)N6)nc(OC[C@]56CCCN5[C@@H](COC(=O)N(C)C)CC6)nc4n3)c12] | 665.32 |
| 154 | ((3R,7aR)-7a-(((4-((1R, 5S)-3,8-diazabicyclo [3.2.1]octan-3-yl)-7-(8- ethyl-7-fluoro-3- hydroxynaphthalen-1- yl)quinazolin-2-yl) oxy)methyl)hexahydro- 1H-pyrrolizin-3-yl) methyl dimethylcarbamate | 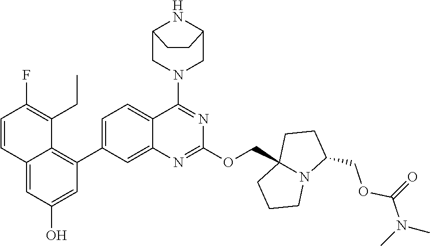 [SMILES: CCc1c(F)ccc2cc(O)cc(-c3ccc4c(N5CC6CCC(C5)N6)nc(OC[C@]56CCCN5[C@@H](COC(=O)N(C)C)CC6)nc4c3)c12] | 669.36 |
| 155 | ((3R,7aR)-7a-(((4-((1R, 5S)-3,8-diazabicyclo [3.2.1]octan-3-yl)-7-(8- ethynyl-7- fluoronaphthalen-1- yl)quinazolin-2-yl)oxy) methyl)hexahydro-1H- pyrrolizin-3-yl)methyl dimethylcarbamate | 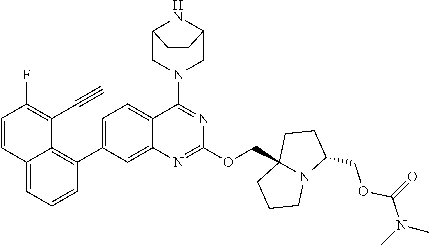 [SMILES: C#Cc1c(F)ccc2cccc(-c3ccc4c(N5CC6CCC(C5)N6)nc(OC[C@]56CCCN5[C@@H](COC(=O)N(C)C)CC6)nc4c3)c12] | 649.32 |
| 156 | 2-(4-((1R,5S)-3,8- diazabicyclo[3.2.1] octan-3-yl)-8-fluoro-2- (((2R,7aS)-2- fluorotetrahydro-1H- pyrrolizin-7a(5H)-yl) methoxy)quinazolin- 7-yl)-1,1a,6,6a- tetrahydrocyclopropa [a]inden-4-ol | 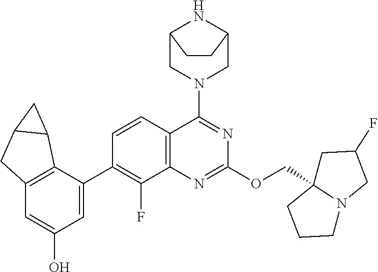 | 560.4 |

### Example 157: synthesis of 4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol

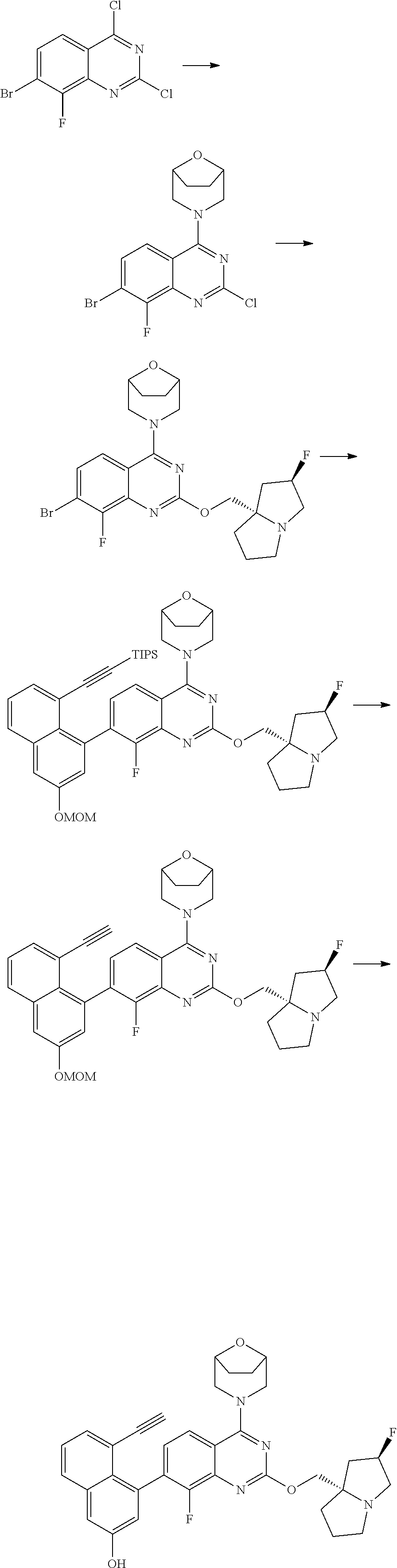

### Step 1: synthesis of (1R,5S)-3-(7-bromo-2-chloro-8-fluoroquinazolin-4-yl)-8-oxa-3-azabicyclo[3.2.1]octane

Dissolve compound 7-bromo-2,4-dichloro-8-fluoroquinazoline (200 mg, 0.68 mmol) and (1R,5S)-8-oxa-3-azabicyclic [3.2.1] octane hydrochloride (112 mg, 0.74 mmol) in anhydrous DMF (5 mL), then add DIEA (262 mg, 2.03 mmol) and stir at room temperature for 1 hour. After completion, dilute the reaction solution with ice water, solid precipitated. Solid was collected with filtering and dried in vacuum to give off-white solid (250 mg, yield: 100%). 1H NMR (400 MHz, CDCl3) δ 7.56-7.44 (m, 2H), 4.47 (d, J=2.0 Hz, 2H), 4.34 (d, J=12.8 Hz, 2H), 3.69 (d, J=11.8 Hz, 2H), 2.06-1.91 (m, 2H), 1.87-1.76 (m, 2H).

### Step 2: synthesis of (1R,5S)-3-(7-bromo-8-fluoro-2-(((2R,7aS)-2-fluorotetrahydro-1H-pyrrolizin-7a(5H)-yl)methoxy)quinazolin-4-yl)-8-oxa-3-azabicyclo[3.2.1]octane

Dissolve compound (2R,7aS)-2-fluorotetrahydro-1H-pyrrolizin-7a (5H)-yl) methanol (139 mg, 0.87 mmol) in anhydrous THF (5 ml), with ice water bath cooling, 60% NaH (34 mg, 0.81 mmol) was added, and stir for 15 minutes under ice water bath cooling Then, compound (1R,5S)-3-(7-bromo-2-chloro-8-fluoroquinazolin-4-yl)-8-oxa-3-azabicyclic [3.2.1] octane (250 mg, 0.67 mmol) was added to the reaction solution, and stirred for 4 hours under ice water bath cooling After the reaction is completed, ice water is added for quenching reaction, extracted with ethyl acetate. The organic layer is washed with brine, dried with sodium sulfate and concentrated. Pre-TLC purification gave white solid (162 mg, yield: 49%). 1H NMR (400 MHz, CDCl3) δ 7.41 (dd, J=9.1, 1.3 Hz, 1H), 7.32-7.27 (m, 1H), 5.30 (dd, J=34.3, 19.6 Hz, 1H), 4.44 (d, J=1.9 Hz, 2H), 4.32-4.19 (m, 3H), 4.12 (d, J=10.3 Hz, 1H), 3.61 (ddd, J=12.9, 5.0, 1.9 Hz, 2H), 3.22 (dd, J=27.4, 14.3 Hz, 3H), 3.03-2.91 (m, 1H), 2.35-2.11 (m, 3H), 2.04-1.77 (m, 7H).

### Step 3: synthesis of (1R,5S)-3-(8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-8-oxa-3-azabicyclo[3.2.1]octane

(1R,5S)-3-(7-bromo-8-fluoro-2-(((2R,7aS)-2-fluorotetrahydro-1H-pyrrolizin-7a(5H)-yl)methoxy)quinazolin-4-yl)-8-oxa-3-azabicyclo[3.2.1]octane (30 mg, 0.06 mmol), triisopropyl((6-(methoxymethoxy)-8-(4,4,5,5-tetramethyl-1,3,2-dioxaborolan-2-yl)naphthalen-1-yl)ethynyl)silane (33 mg, 0.07 mmol), cesium carbonate (50 mg, 0.15 mmol) and Pd(dppf)Cl₂(9 mg, 0.01 mmol) were added to 1,4-dioxane/ [symbol] =5/1 (3 mL). Run evacuating and backfilling with nitrogen, the reaction was stirred at 100° C. for 12 hours. After completion, the reaction was cooled to room temperature and diluted with ethyl acetate. The organic layer was dried over sodium sulfate and concentrated. Prep-TLC gave off-white solid (40 mg, yield: 83.6%). MS m/z: 783.7 [M+H]+

### Step 4: synthesis of (1R,5S)-3-(7-(8-ethynyl-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-8-oxa-3-azabicyclo[3.2.1]octane

(1R,5S)-3-(8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-8-oxa-3-azabicyclo[3.2.1]octane (40 mg, 0.05 mmol) was added in DMF (3 mL). CsF (39 mg, 0.26 mmol) was added. The reaction was stirred at room temperature for 2 hours. After completion, the reaction was diluted with water and extracted with ethyl acetate. The organic layer was dried over sodium sulfate and concentrated. The resulting crude product was used directly in next step (32 mg, yield: 100%).

### Step 5: synthesis of 4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol

(1R,5S)-3-(7-(8-ethynyl-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-8-oxa-3-azabicyclo[3.2.1]octane (32 mg, 0.05 mmol) was dissolved in acetonitrile (1 mL). 4N HCl in 1,4-dioxane (1 mL) was added. Three reaction was stirred at room temperature for 1 hours. After completion, carefully add saturated aqueous sodium bicarbonate to adjust pH to 7. Run extraction with DCM, the organic layer was dried over sodium sulfate and concentrated. Prep-TLC purification gave off-white solid (18 mg, yield: 61%). 1H NMR (400 MHz, CDCl3) δ 7.69 (d, J=8.2 Hz, 1H), 7.46 (t, J=6.8 Hz, 1H), 7.30-7.20 (m, 4H), 7.06 (dd, J=15.0, 7.1 Hz, 1H), 5.39 (d, J=11.3 Hz, 0.5H), 5.27 (d, J=13.6 Hz, 0.5H), 4.44-4.20 (m, 6H), 3.63-3.56 (m, 2H), 3.40-3.33 (m, 2H), 3.27-3.12 (m, 1H), 3.08-2.98 (m, 2H), 2.41 (d, J=2.8 Hz, 1H), 2.31-2.09 (m, 3H), 2.07-1.73 (m, 5H). MS m/z: 583.57 [M+H]+.

The compounds of Examples 158-169 were prepared by the preparation method for Example 157

| Ex. | Compound name | Structural formula | m/z: ES⁺[M + H] |
|---|---|---|---|
| 158 | 4-(4-((1R,5S)-8-thia-3-azabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-5-ethynyl-6- fluoronaphthalen-2-ol | 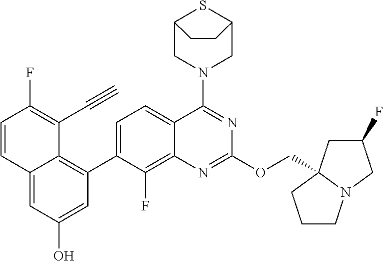 [SMILES: C#Cc1c(F)ccc2cc(O)cc(-c3ccc4c(N5CC6CCC(C5)S6)nc(OC[C@@]56CCCN5C[C@H](F)C6)nc4c3F)c12] | 617 |
| 159 | (1R,5S,8R)-3-(7-(8-ethynyl-7-fluoro-3- hydroxynaphthalen-1-yl)-8-fluoro-2-(((2R,7aS)-2- fluorotetrahydro-1H-pyrrolizin-7a(5H)- yl)methoxy)quinazolin-4-yl)-8-thia-3- azabicyclo[3.2.1]octane 8-oxide | 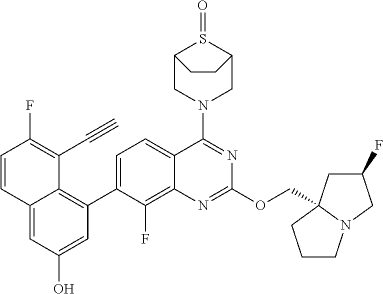 [SMILES: C#Cc1c(F)ccc2cc(O)cc(-c3ccc4c(N5CC6CCC(C5)S6=O)nc(OC[C@@]56CCCN5C[C@H](F)C6)nc4c3F)c12] | 633 |
| 160 | (1R,5S)-3-(7-(8-ethynyl-7-fluoro-3- hydroxynaphthalen-1-yl)-8-fluoro-2-(((2R,7aS)-2- fluorotetrahydro-1H-pyrrolizin-7a(5H)- yl)methoxy)quinazolin-4-yl)-8-thia-3- azabicyclo[3.2.1]octane 8,8-dioxide | 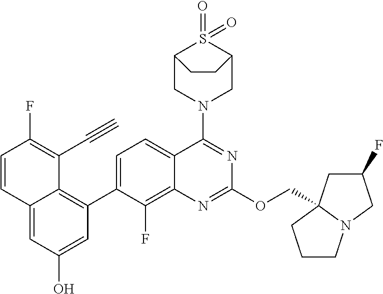 [SMILES: C#Cc1c(F)ccc2cc(O)cc(-c3ccc4c(N5CC6CCC(C5)S6(=O)=O)nc(OC[C@@]56CCCN5C[C@H](F)C6)nc4c3F)c12] | 649 |
| 161 | 4-(4-((1R,5S)-8-thia-3-azabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-5-ethyl-6- fluoronaphthalen-2-ol | 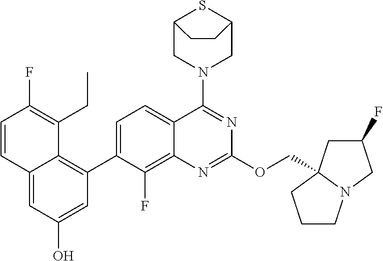 [SMILES: CCc1c(F)ccc2cc(O)cc(-c3ccc4c(N5CC6CCC(C5)S6)nc(OC[C@@]56CCCN5C[C@H](F)C6)nc4c3F)c12] | 621.7 |
| 162 | (1R,5S,8R)-3-(7-(8-ethyl-7-fluoro-3- hydroxynaphthalen-1-yl)-8-fluoro-2-(((2R,7aS)-2- fluorotetrahydro-1H-pyrrolizin-7a(5H)- yl)methoxy)quinazolin-4-yl)-8-thia-3- azabicyclo[3.2.1]octane 8-oxide | 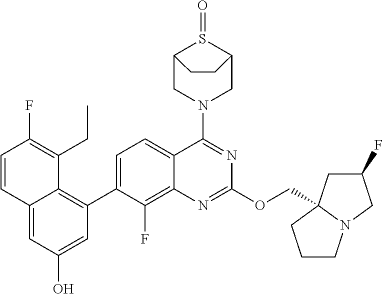 [SMILES: CCc1c(F)ccc2cc(O)cc(-c3ccc4c(N5CC6CCC(C5)S6=O)nc(OC[C@@]56CCCN5C[C@H](F)C6)nc4c3F)c12] | 637 |
| 163 | (1R,5S)-3-(7-(8-ethyl-7-fluoro-3-hydroxynaphthalen-1- yl)-8-fluoro-2-(((2R,7aS)-2-fluorotetrahydro-1H- pyrrolizin-7a(5H)-yl)methoxy)quinazolin-4-yl)-8-thia- 3-azabicyclo[3.2.1]octane 8,8-dioxide | 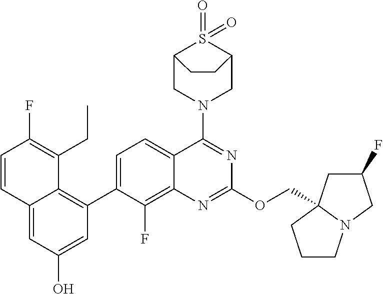 [SMILES: CCc1c(F)ccc2cc(O)cc(-c3ccc4c(N5CC6CCC(C5)S6(=O)=O)nc(OC[C@@]56CCCN5C[C@H](F)C6)nc4c3F)c12] | 653 |
| 164 | 4-(4-((1R,5S)-8-thia-3-azabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-5,6- difluoronaphthalen-2-ol | 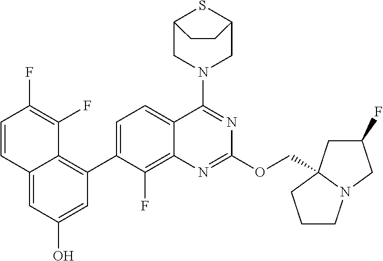 [SMILES: Oc1cc(-c2ccc3c(N4CC5CCC(C4)S5)nc(OC[C@@]45CCCN4C[C@H](F)C5)nc3c2F)c2c(F)c(F)ccc2c1] | 611 |
| 165 | (1R,5S,8R)-3-(7-(7,8-difluoro-3-hydroxynaphthalen-1- yl)-8-fluoro-2-(((2R,7aS)-2-fluorotetrahydro-1H- pyrrolizin-7a(5H)-yl)methoxy)quinazolin-4-yl)-8-thia- 3-azabicyclo[3.2.1]octane 8-oxide | 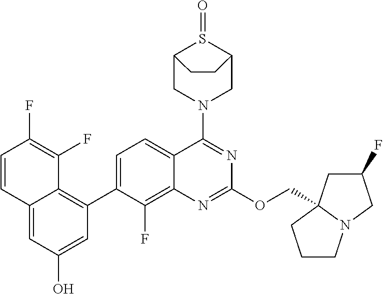 [SMILES: O=S1C2CCC1CN(c1nc(OC[C@@]34CCCN3C[C@H](F)C4)nc3c(F)c(-c4cc(O)cc5ccc(F)c(F)c45)ccc13)C2] | 627 |
| 166 | (1R,5S)-3-(7-(7,8-difluoro-3-hydroxynaphthalen-1-yl)- 8-fluoro-2-(((2R,7aS)-2-fluorotetrahydro-1H- pyrrolizin-7a(5H)-yl)methoxy)quinazolin-4-yl)-8-thia- 3-azabicyclo[3.2.1]octane 8,8-dioxide | 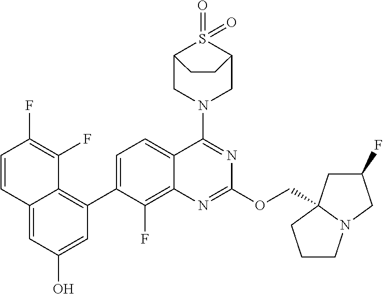 [SMILES: O=S1(=O)C2CCC1CN(c1nc(OC[C@@]34CCCN3C[C@H](F)C4)nc3c(F)c(-c4cc(O)cc5ccc(F)c(F)c45)ccc13)C2] | 643 |
| 167 | 4-(4-((1R,5S)-8-thia-3-azabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-5- fluoronaphthalen-2-ol | 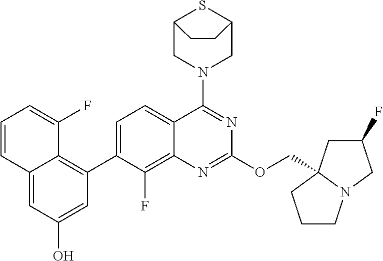 [SMILES: Oc1cc(-c2ccc3c(N4CC5CCC(C4)S5)nc(OC[C@@]45CCCN4C[C@H](F)C5)nc3c2F)c2c(F)cccc2c1] | 593 |
| 168 | (1R,5S,8R)-3-(8-fluoro-7-(8-fluoro-3- hydroxynaphthalen-1-yl)-2-(((2R,7aS)-2- fluorotetrahydro-1H-pyrrolizin-7a(5H)- yl)methoxy)quinazolin-4-yl)-8-thia-3- azabicyclo[3.2.1]octane 8-oxide | 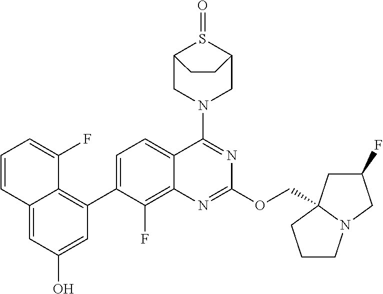 [SMILES: O=S1C2CCC1CN(c1nc(OC[C@@]34CCCN3C[C@H](F)C4)nc3c(F)c(-c4cc(O)cc5cccc(F)c45)ccc13)C2] | 609 |
| 169 | (1R,5S)-3-(8-fluoro-7-(8-fluoro-3-hydroxynaphthalen- 1-yl)-2-(((2R,7aS)-2-fluorotetrahydro-1H-pyrrolizin- 7a(5H)-yl)methoxy)quinazolin-4-yl)-8-thia-3- azabicyclo[3.2.1]octane 8,8-dioxide | 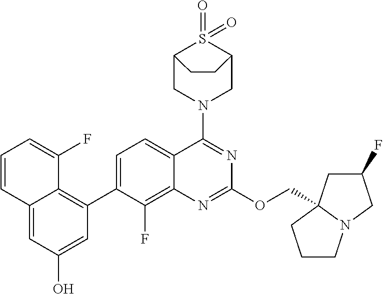 [SMILES: O=S1(=O)C2CCC1CN(c1nc(OC[C@@]34CCCN3C[C@H](F)C4)nc3c(F)c(-c4cc(O)cc5cccc(F)c45)ccc13)C2] | 625 |

### Example 170: synthesis of 4-((1R,5S)-3,8-diazabicyclo[3.2.1]octan-3-yl)-7-(8-chloro-7-fluoronaphthalen-1-yl)-2-(((2R,7aS)-2-fluorotetrahydro-1H-pyrrolizin-7a(5H)-yl)methoxy)quinoline-3-carbonitrile

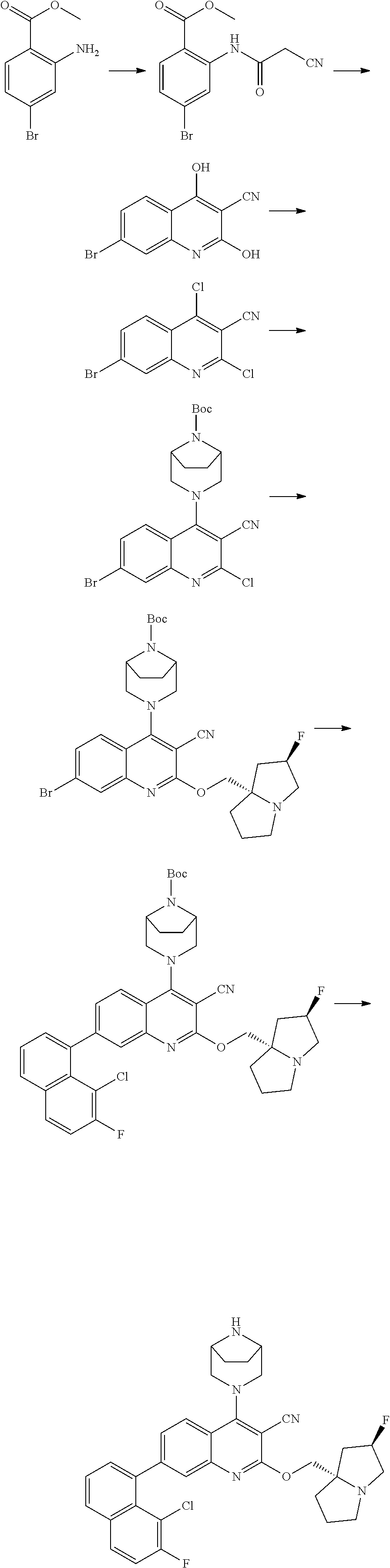

### Step 1: synthesis of methyl 4-bromo-2-(2-cyanoacetamido)benzoate

methyl 2-amino-4-bromobenzoate (10 g, 43.47 mmol) and cyanoacetic acid (4.44 g, 52.16 mmol) were dissolve in DCM (100 mL). With ice-water bath cooling, EDCI (12.5 g, 65.10 mmol) was added. The reaction was stirred in ice-water bath for 1 hours. After completion, water was added and the mixture was extracted with DCM. The organic layer was washed with brine, dried over sodium sulfate and concentrated to give target compound (12.9 g, yield: 100%). 1HNMR (400 MHz, CDCl3) δ 11.73 (s, 1H), 8.88 (d, J=1.5 Hz, 1H), 7.92 (d, J=8.6 Hz, 1H), 7.32 (dd, J=8.6, 1.8 Hz, 1H), 3.97 (s, 3H), 3.61 (s, 2H).

### Step 2: synthesis of 7-bromo-2,4-dihydroxyquinoline-3-carbonitrile

methyl 4-bromo-2-(2-cyanoacetamido)benzoate (12.9 g, 43.42 mmol) was dissolved in anhydrous methanol (100 mL). With ice-water bath cooling, a solution of 30% sodium methoxide (11.73 g, 65.13 mmol) was added. The reaction was stirred in ice-water bath for 30 min. After completion, the reaction was treated with 10% aqueous HCl to adjust pH to 2-3. 100 mL of water was added. The precipitated solid was collected by filtering and dried under vacuum to give off-white solid (11.18 g, yield: 97%). 1HNMR (400 MHz, DMSO) δ 11.62 (s, 1H), 7.90 (d, J=8.6 Hz, 1H), 7.45 (d, J=1.8 Hz, 1H), 7.37 (dd, J=8.6, 1.8 Hz, 1H).

### Step 3: synthesis of 7-bromo-2,4-dichloroquinoline-3-carbonitrile

7-bromo-2,4-dihydroxyquinoline-3-carbonitrile (11.0 g, 41.50 mmol) was dissolve in acetonitrile (10 mL) and POCl3 (40 mL). The reaction was heated at 90° C. and stirred for 16 hours. After completion, the reaction was cooled to room temperature and concentrated to give light yellow solid which was used directly in next step (12.53 g, yield: 100%). 1HNMR (400 MHz, CDCl3) δ 8.28 (d, J=1.8 Hz, 1H), 8.13 (d, J=8.9 Hz, 1H), 7.87 (dd, J=9.0, 1.9 Hz, 1H).

### Step 4: synthesis of tert-butyl (1R,5S)-3-(7-bromo-2-chloro-3-cyanoquinoline-4-yl)-3,8-diazabicyclo[3.2.1]octane-8-carboxylate

7-bromo-2,4-dichloroquinoline-3-carbonitrile (5.00 g, 16.56 mmol) was dissolved in anhydrous DMF (50 mL). With ice-water bath cooling, DIEA (12.84 g, 16.42 mL, 99.36 mmol) and tert-butyl (1R,5S)-3,8-diazabicyclo[3.2.1]octane-8-carboxylate (3.87 g, 18.22 mmol) were added successively. The reaction was stirred in ice-water bath for 10 min. After completion, 100 mL of cooled water was added under stirring. The solid was collected by filtering and eluted with water. Drying under vacuum gave light yellow solid which was used directly in next step (7.2 g, yield: 88.59%). 1HNMR (400 MHz, CDCl3) δ 8.18 (d, J=1.9 Hz, 1H), 8.05 (d, J=9.0 Hz, 1H), 7.67 (dd, J=9.0, 2.0 Hz, 1H), 4.43 (s, 2H), 3.90 (d, J=49.2 Hz, 2H), 3.41 (s, 2H), 2.24-2.06 (m, 4H), 1.53 (s, 9H).

### Step 5: synthesis of tert-butyl (1R,5S)-3-(7-bromo-3-cyano-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

((2R,7aS)-2-fluorotetrahydro-1H-pyrrolizin-7a(5H)-yl)methanol (44 mg, 0.27 mmol) was dissolved in anhydrous THF (3 ml). With ice-water bath cooling, 60% NaH (11 mg, 0.27 mmol) was added. The reaction was stirred in ice-water bath for 15 min. tert-butyl (1R,5S)-3-(7-bromo-2-chloro-3-cyanoquinolin-4-yl)-3,8-diazabicyclo[3.2.1]octane-8-carboxylate (100 mg, 0.21 mmol) was added. The stirring was kept in ice-water bath for 4 hours. After completion, the reaction was quenched by addition of water and extracted with ethyl acetate. The organic layer was washed with brine, dried over sodium sulfate and concentrated. Purification with prep-TLC gave off-white solid (120 mg, yield: 95%). 1HNMR (400 MHz, CDCl3) δ 7.95 (dd, J=9.4, 5.5 Hz, 2H), 7.47 (dd, J=8.9, 2.0 Hz, 1H), 5.37 (d, J=52.7 Hz, 1H), 4.34 (d, J=41.9 Hz, 4H), 3.90 (s, 1H), 3.76-3.62 (m, 1H), 3.28 (s, 5H), 3.03 (s, 1H), 2.13 (dd, J=34.9, 7.1 Hz, 10H), 1.52 (s, 9H).

### Step 6: synthesis of tert-butyl (1R,5S)-3-(7-(8-chloro-7-fluoronaphthalen-1-yl)-3-cyano-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

tert-butyl (1R,5S)-3-(7-bromo-3-cyano-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (50 mg, 0.08 mmol), 2-(8-chloro-7-fluoronaphthalen-1-yl)-4,4,5,5-tetramethyl-1,3,2-dioxaborolane (31 mg, 0.10 mmol), cesium carbonate (68 mg, 0.21 mmol) and Pd(PPh3)4 (28 mg, 0.02 mmol) were added to 1,4-dioxane/water=5/1(3 mL). Run evacuating and backfilling with nitrogen. The reaction was heated at 100° C. under nitrogen atmosphere for 6 hours. After completion, the reaction was cooled to room temperature. Water was added and the reaction was extracted with ethyl acetate. The organic layer was dried over sodium sulfate and concentrated. Purification with prep-TLC gave off-white solid (43 mg, yield: 73%).

### Step 7: synthesis of 4-((1R,5S)-3,8-diazabicyclo[3.2.1]octan-3-yl)-7-(8-chloro-7-fluoronaphthalen-1-yl)-2-(((2R,7aS)-2-fluorotetrahydro-1H-pyrrolizin-7a(5H)-yl)methoxy)quinoline-3-carbonitrile

tert-butyl (1R,5S)-3-(7-(8-chloro-7-fluoronaphthalen-1-yl)-3-cyano-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (43 mg, 0.06 mmol) was dissolved in (3 mL). TFA (1 mL) was added. The reaction was stirred at room temperature for 1 hour. After completion, the reaction solution was concentrated. Then, saturated aqueous sodium bicarbonate and water were added. The organic layer was separated out, dried over sodium sulfate and concentrated. Purification with prep-TLC gave off-white solid (30 mg, yield: 81%). 1H NMR (400 MHz, CDCl3) δ 8.12 (d, J=8.6 Hz, 1H), 7.95-7.85 (m, 2H), 7.76 (s, 1H), 7.56-7.49 (m, 1H), 7.46 (t, J=6.4 Hz, 1H), 7.40 (d, J=8.6 Hz, 1H), 7.36 (dd, J=8.6, 1.8 Hz, 1H), 5.37 (d, J=45.4 Hz, 1H), 4.32 (d, J=12.0 Hz, 2H), 3.95-3.74 (m, 2H), 3.70 (s, 2H), 3.50 (ddd, J=31.9, 21.2, 11.7 Hz, 6H), 3.25 (s, 1H), 3.03 (s, 1H), 2.44-2.14 (m, 5H), 2.12-2.06 (m, 3H). MS m/z: 600.5 [M+H]+

The compounds of Examples 171-198 were prepared by the preparation method for Example 170

| Ex. | Compound name | Structural formula | m/z: ES⁺[M + H] |
|---|---|---|---|
| 171 | 4-((1R,5S)-3,8-diazabicyclo[3.2.1]octan-3-yl)-7-(8- ethynyl-7-fluoro-3-hydroxynaphthalen-1-yl)-2- (((2R,7aS)-2-fluorotetrahydro-1H-pyrrolizin-7a(5H)- yl)methoxy)quinoline-3-carbonitrile | 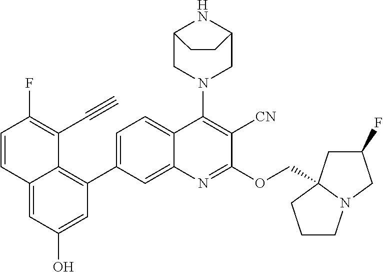 [SMILES: C#Cc1c(F)ccc2cc(O)cc(-c3ccc4c(N5CC6CCC(C5)N6)c(C#N)c(OC[C@@]56CCCN5C[C@H](F)C6)nc4c3)c12] | 606.7 |
| 172 | 4-((1R,5S)-3,8-diazabicyclo[3.2.1]octan-3-yl)-7-(8- ethyl-7-fluoro-3-hydroxynaphthalen-1-yl)-2- (((2R,7aS)-2-fluorotetrahydro-1H-pyrrolizin-7a(5H)- yl)methoxy)quinoline-3-carbonitrile | 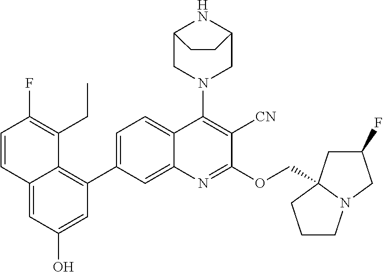 [SMILES: CCc1c(F)ccc2cc(O)cc(-c3ccc4c(N5CC6CCC(C5)N6)c(C#N)c(OC[C@@]56CCCN5C[C@H](F)C6)nc4c3)c12] | 610.7 |
| 173 | 4-((1R,5S)-3,8-diazabicyclo[3.2.1]octan-3-yl)-7-(8- ethynyl-3-hydroxynaphthalen-1-yl)-2-(((2R,7aS)-2- fluorotetrahydro-1H-pyrrolizin-7a(5H)- yl)methoxy)quinoline-3-carbonitrile | 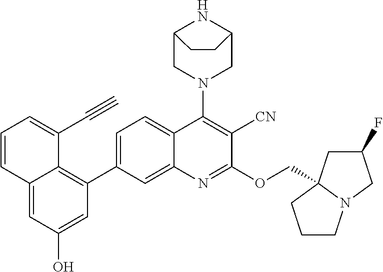 [SMILES: C#Cc1cccc2cc(O)cc(-c3ccc4c(N5CC6CCC(C5)N6)c(C#N)c(OC[C@@]56CCCN5C[C@H](F)C6)nc4c3)c12] | 588.7 |
| 174 | 4-((1R,5S)-3,8-diazabicyclo[3.2.1]octan-3-yl)-7-(8- ethyl-3-hydroxynaphthalen-1-yl)-2-(((2R,7aS)-2- fluorotetrahydro-1H-pyrrolizin-7a(5H)- yl)methoxy)quinoline-3-carbonitrile | 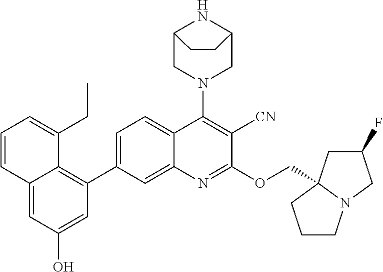 [SMILES: CCc1cccc2cc(O)cc(-c3ccc4c(N5CC6CCC(C5)N6)c(C#N)c(OC[C@@]56CCCN5C[C@H](F)C6)nc4c3)c12] | 592.7 |
| 175 | 4-((1R,5S)-3,8-diazabicyclo[3.2.1]octan-3-yl)-7-(7,8- difluoro-3-hydroxynaphthalen-1-yl)-2-(((2R,7aS)-2- fluorotetrahydro-1H-pyrrolizin-7a(5H)- yl)methoxy)quinoline-3-carbonitrile | 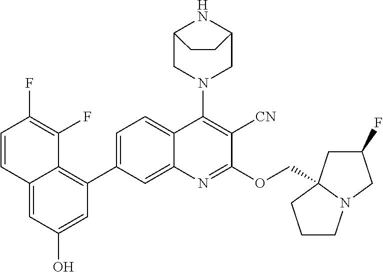 [SMILES: N#Cc1c(OC[C@@]23CCCN2C[C@H](F)C3)nc2cc(-c3cc(O)cc4ccc(F)c(F)c34)ccc2c1N1CC2CCC(C1)N2] | 600.6 |
| 176 | 4-((1R,5S)-3,8-diazabicyclo[3.2.1]octan-3-yl)-7-(8- fluoro-3-hydroxynaphthalen-1-yl)-2-(((2R,7aS)-2- fluorotetrahydro-1H-pyrrolizin-7a(5H)- yl)methoxy)quinoline-3-carbonitrile | 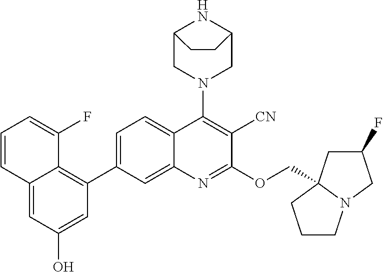 [SMILES: N#Cc1c(OC[C@@]23CCCN2C[C@H](F)C3)nc2cc(-c3cc(O)cc4cccc(F)c34)ccc2c1N1CC2CCC(C1)N2] | 582.6 |
| 177 | 4-((1R,5S)-3,8-diazabicyclo[3.2.1]octan-3-yl)-7-(8- ethynyl-7-fluoro-3-hydroxynaphthalen-1-yl)-8-fluoro- 2-(((2R,7aS)-2-fluorotetrahydro-1H-pyrrolizin-7a(5H)- yl)methoxy)quinoline-3-carbonitrile | 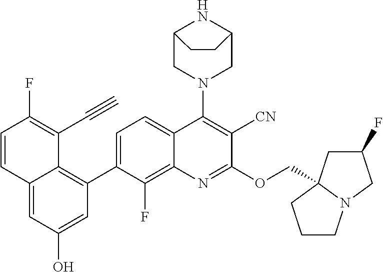 [SMILES: C#Cc1c(F)ccc2cc(O)cc(-c3ccc4c(N5CC6CCC(C5)N6)c(C#N)c(OC[C@@]56CCCN5C[C@H](F)C6)nc4c3F)c12] | 624.6 |
| 178 | 4-((1R,5S)-3,8-diazabicyclo[3.2.1]octan-3-yl)-7-(8- ethyl-7-fluoro-3-hydroxynaphthalen-1-yl)-8-fluoro-2- (((2R,7aS)-2-fluorotetrahydro-1H-pyrrolizin-7a(5H)- yl)methoxy)quinoline-3-carbonitrile | 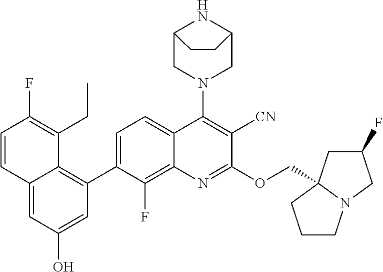 [SMILES: CCc1c(F)ccc2cc(O)cc(-c3ccc4c(N5CC6CCC(C5)N6)c(C#N)c(OC[C@@]56CCCN5C[C@H](F)C6)nc4c3F)c12] | 628.7 |
| 179 | 4-((1R,5S)-3,8-diazabicyclo[3.2.1]octan-3-yl)-7-(7,8- difluoro-3-hydroxynaphthalen-1-yl)-8-fluoro-2- (((2R,7aS)-2-fluorotetrahydro-1H-pyrrolizin-7a(5H)- yl)methoxy)quinoline-3-carbonitrile | 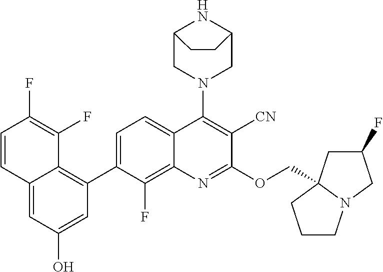 [SMILES: N#Cc1c(OC[C@@]23CCCN2C[C@H](F)C3)nc2c(F)c(-c3cc(O)cc4ccc(F)c(F)c34)ccc2c1N1CC2CCC(C1)N2] | 618.6 |
| 180 | 4-((1R,5S)-3,8-diazabicyclo[3.2.1]octan-3-yl)-8- fluoro-7-(8-fluoro-3-hydroxynaphthalen-1-yl)-2- (((2R,7aS)-2-fluorotetrahydro-1H-pyrrolizin-7a(5H)- yl)methoxy)quinoline-3-carbonitrile | 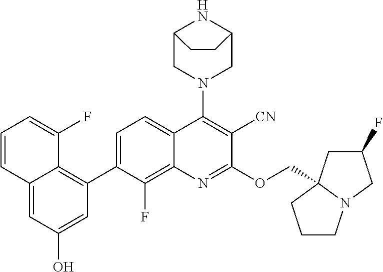 [SMILES: N#Cc1c(OC[C@@]23CCCN2C[C@H](F)C3)nc2c(F)c(-c3cc(O)cc4cccc(F)c34)ccc2c1N1CC2CCC(C1)N2] | 600.6 |
| 181 | 4-((1R,5S)-3,8-diazabicyclo[3.2.1]octan-3-yl)-7-(8- ethyl-3-hydroxynaphthalen-1-yl)-8-fluoro-2- (((2R,7aS)-2-fluorotetrahydro-1H-pyrrolizin-7a(5H)- yl)methoxy)quinoline-3-carbonitrile | 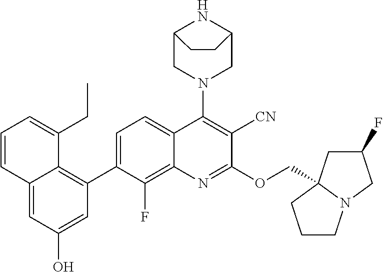 [SMILES: CCc1cccc2cc(O)cc(-c3ccc4c(N5CC6CCC(C5)N6)c(C#N)c(OC[C@@]56CCCN5C[C@H](F)C6)nc4c3F)c12] | 610 |
| 182 | 4-((1R,5S)-3,8-diazabicyclo[3.2.1]octan-3-yl)-7-(8- ethynyl-3-hydroxynaphthalen-1-yl)-8-fluoro-2- (((2R,7aS)-2-fluorotetrahydro-1H-pyrrolizin-7a(5H)- yl)methoxy)quinoline-3-carbonitrile | 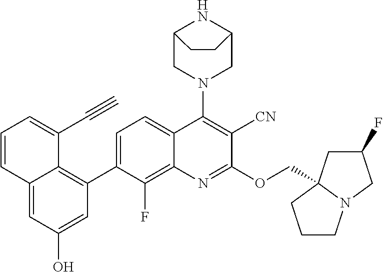 [SMILES: C#Cc1cccc2cc(O)cc(-c3ccc4c(N5CC6CCC(C5)N6)c(C#N)c(OC[C@@]56CCCN5C[C@H](F)C6)nc4c3F)c12] | 606 |
| 183 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)-3-(trifluoromethyl)quinolin-7-yl)-5- ethynyl-6-fluoronaphthalen-2-ol | 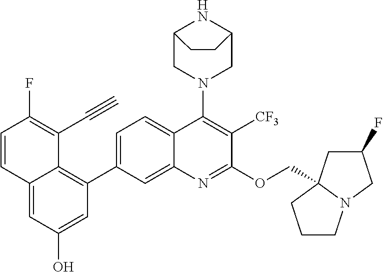 [SMILES: C#Cc1c(F)ccc2cc(O)cc(-c3ccc4c(N5CC6CCC(C5)N6)c(C(F)(F)F)c(OC[C@@]56CCCN5C[C@H](F)C6)nc4c3)c12] | 649 |
| 184 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)-3-(trifluoromethyl)quinolin-7-yl)-5- ethynylnaphthalen-2-ol | 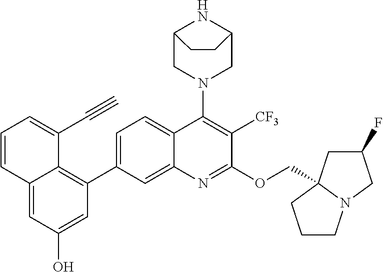 [SMILES: C#Cc1cccc2cc(O)cc(-c3ccc4c(N5CC6CCC(C5)N6)c(C(F)(F)F)c(OC[C@@]56CCCN5C[C@H](F)C6)nc4c3)c12] | 631 |
| 185 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)quinolin-7-yl)- 5-ethynyl-6-fluoronaphthalen-2-ol | 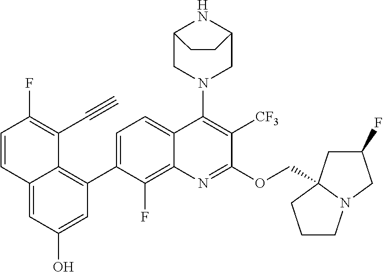 [SMILES: C#Cc1c(F)ccc2cc(O)cc(-c3ccc4c(N5CC6CCC(C5)N6)c(C(F)(F)F)c(OC[C@@]56CCCN5C[C@H](F)C6)nc4c3F)c12] | 667 |
| 186 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)quinolin-7-yl)- 5-ethyl-6-fluoronaphthalen-2-ol | 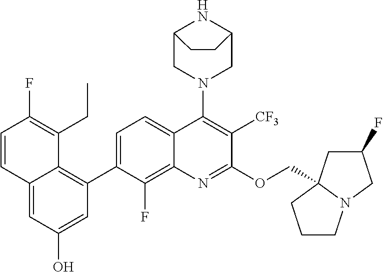 [SMILES: CCc1c(F)ccc2cc(O)cc(-c3ccc4c(N5CC6CCC(C5)N6)c(C(F)(F)F)c(OC[C@@]56CCCN5C[C@H](F)C6)nc4c3F)c12] | 671 |
| 187 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)quinolin-7-yl)- 5,6-difluoronaphthalen-2-ol | 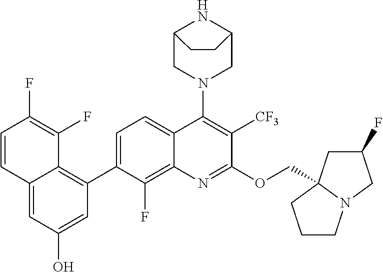 [SMILES: Oc1cc(-c2ccc3c(N4CC5CCC(C4)N5)c(C(F)(F)F)c(OC[C@@]45CCCN4C[C@H](F)C5)nc3c2F)c2c(F)c(F)ccc2c1] | 661 |
| 188 | 4-((1R,5S)-3,8-diazabicyclo[3.2.1]octan-3-yl)-7-(8- ethynyl-7-fluoro-3-hydroxynaphthalen-1-yl)-2- (((2R,7aS)-2-fluorotetrahydro-1H-pyrrolizin-7a(5H)- yl)methoxy)-1,6-naphthyridine-3-carbonitrile | 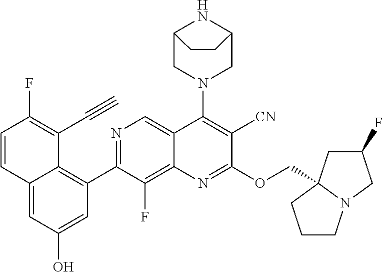 [SMILES: C#Cc1c(F)ccc2cc(O)cc(-c3ncc4c(N5CC6CCC(C5)N6)c(C#N)c(OC[C@@]56CCCN5C[C@H](F)C6)nc4c3F)c12] | 607 |
| 189 | 4-((1R,5S)-3,8-diazabicyclo[3.2.1]octan-3-yl)-7-(8- ethynyl-3-hydroxynaphthalen-1-yl)-8-fluoro-2- (((2R,7aS)-2-fluorotetrahydro-1H-pyrrolizin-7a(5H)- yl)methoxy)-1,6-naphthyridine-3-carbonitrile | 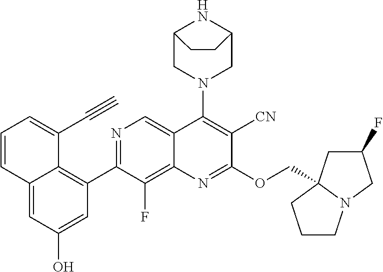 [SMILES: C#Cc1cccc2cc(O)cc(-c3ncc4c(N5CC6CCC(C5)N6)c(C#N)c(OC[C@@]56CCCN5C[C@H](F)C6)nc4c3F)c12] | 607 |
| 190 | 4-((1R,5S)-3,8-diazabicyclo[3.2.1]octan-3-yl)-7-(8- ethyl-3-hydroxynaphthalen-1-yl)-8-fluoro-2- (((2R,7aS)-2-fluorotetrahydro-1H-pyrrolizin-7a(5H)- yl)methoxy)-1,6-naphthyridine-3-carbonitrile | 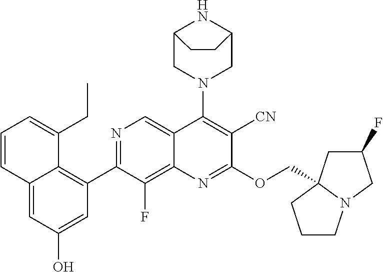 [SMILES: CCc1cccc2cc(O)cc(-c3ncc4c(N5CC6CCC(C5)N6)c(C#N)c(OC[C@@]56CCCN5C[C@H](F)C6)nc4c3F)c12] | 611 |
| 191 | 4-((1R,5S)-3,8-diazabicyclo[3.2.1]octan-3-yl)-7-(8- ethynyl-7-fluoro-3-hydroxynaphthalen-1-yl)-8-fluoro- 2-(((2R,7aS)-2-fluorotetrahydro-1H-pyrrolizin-7a(5H)- yl)methoxy)-1,6-naphthyridine-3-carbonitrile | 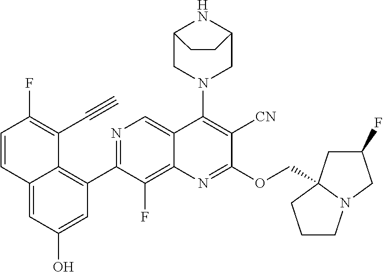 [SMILES: C#Cc1c(F)ccc2cc(O)cc(-c3ncc4c(N5CC6CCC(C5)N6)c(C#N)c(OC[C@@]56CCCN5C[C@H](F)C6)nc4c3F)c12] | 625.6 |
| 192 | 4-((1R,5S)-3,8-diazabicyclo[3.2.1]octan-3-yl)-7-(8- ethyl-7-fluoro-3-hydroxynaphthalen-1-yl)-8-fluoro-2- (((2R,7aS)-2-fluorotetrahydro-1H-pyrrolizin-7a(5H)- yl)methoxy)-1,6-naphthyridine-3-carbonitrile | 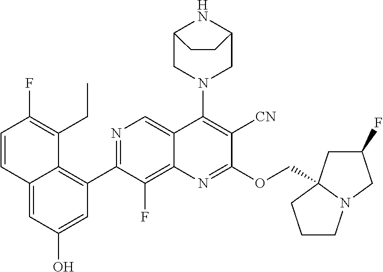 [SMILES: CCc1c(F)ccc2cc(O)cc(-c3ncc4c(N5CC6CCC(C5)N6)c(C#N)c(OC[C@@]56CCCN5C[C@H](F)C6)nc4c3F)c12] | 629 |
| 193 | 4-((1R,5S)-3,8-diazabicyclo[3.2.1]octan-3-yl)-7-(7,8- difluoro-3-hydroxynaphthalen-1-yl)-8-fluoro-2- (((2R,7aS)-2-fluorotetrahydro-1H-pyrrolizin-7a(5H)- yl)methoxy)-1,6-naphthyridine-3-carbonitrile | 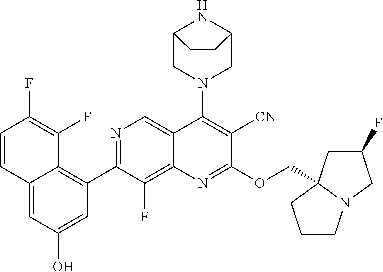 [SMILES: N#Cc1c(OC[C@@]23CCCN2C[C@H](F)C3)nc2c(F)c(-c3cc(O)cc4ccc(F)c(F)c34)ncc2c1N1CC2CCC(C1)N2] | 619.6 |
| 194 | 4-((1R,5S)-3,8-diazabicyclo[3.2.1]octan-3-yl)-8- fluoro-7-(8-fluoro-3-hydroxynaphthalen-1-yl)-2- (((2R,7aS)-2-fluorotetrahydro-1H-pyrrolizin-7a(5H)- yl)methoxy)-1,6-naphthyridine-3-carbonitrile | 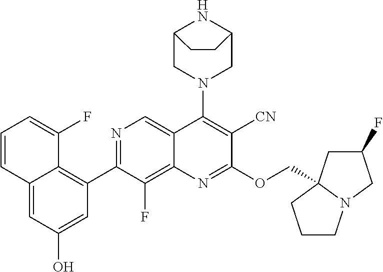 [SMILES: N#Cc1c(OC[C@@]23CCCN2C[C@H](F)C3)nc2c(F)c(-c3cc(O)cc4cccc(F)c34)ncc2c1N1CC2CCC(C1)N2] | 601.6 |
| 195 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)-3-(trifluoromethyl)-1,6-naphthyridin-7- yl)-5-ethynyl-6-fluoronaphthalen-2-ol | 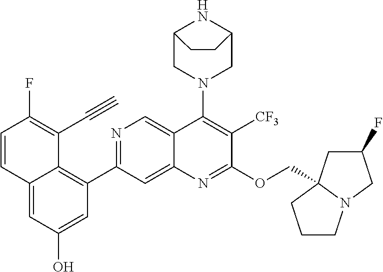 [SMILES: C#Cc1c(F)ccc2cc(O)cc(-c3cc4nc(OC[C@@]56CCCN5C[C@H](F)C6)c(C(F)(F)F)c(N5CC6CCC(C5)N6)c4cn3)c12] | 650 |
| 196 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)-1,6- naphthyridin-7-yl)-5-ethynyl-6-fluoronaphthalen-2-ol | 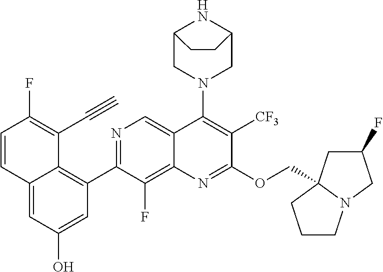 [SMILES: C#Cc1c(F)ccc2cc(O)cc(-c3ncc4c(N5CC6CCC(C5)N6)c(C(F)(F)F)c(OC[C@@]56CCCN5C[C@H](F)C6)nc4c3F)c12] | 668 |
| 197 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)-1,6- naphthyridin-7-yl)-5-ethyl-6-fluoronaphthalen-2-ol | 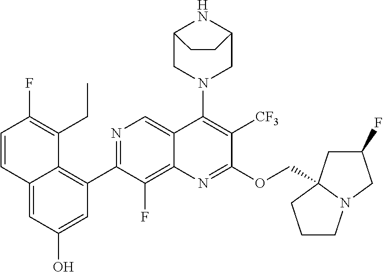 [SMILES: CCc1c(F)ccc2cc(O)cc(-c3ncc4c(N5CC6CCC(C5)N6)c(C(F)(F)F)c(OC[C@@]56CCCN5C[C@H](F)C6)nc4c3F)c12] | 672 |
| 198 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-3-(trifluoromethyl)-1,6- naphthyridin-7-yl)-5,6-difluoronaphthalen-2-ol | 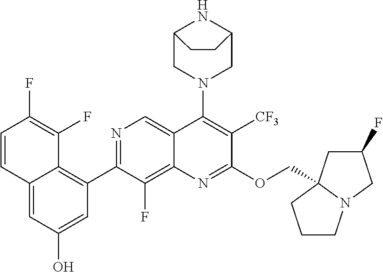 [SMILES: Oc1cc(-c2ncc3c(N4CC5CCC(C4)N5)c(C(F)(F)F)c(OC[C@@]45CCCN4C[C@H](F)C5)nc3c2F)c2c(F)c(F)ccc2c1] | 662 |

### Example 199: synthesis of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-5,6-difluoronaphthalen-2-ol

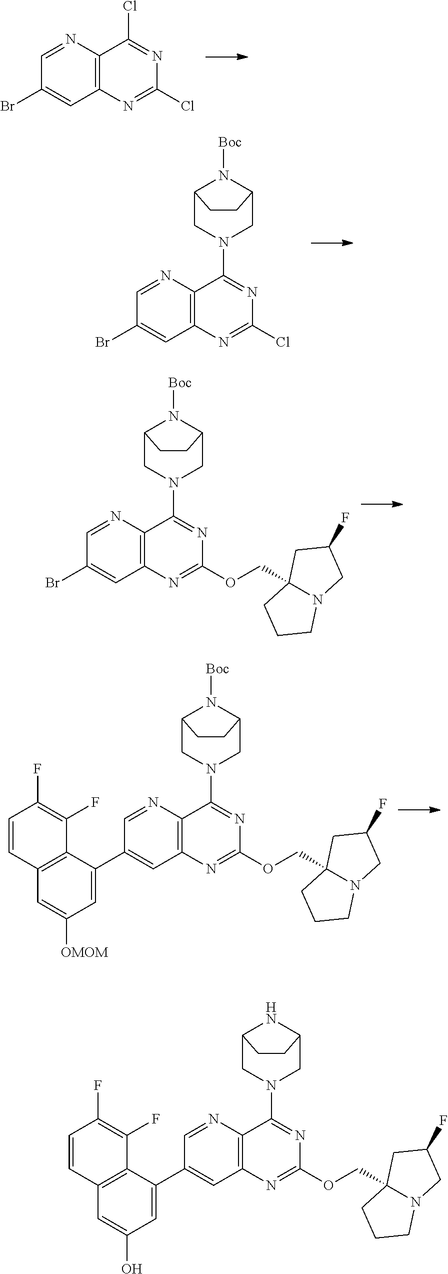

### Step 1: synthesis of tert-butyl (1R,5S)-3-(7-bromo-2-chloropyrido[3,2-d]pyrimidin-4-yl)-3,8-diazabicyclo[3.2.1]octane-8-carboxylate

A solution of 7-bromo-2,4-dichloropyrido[3,2-d]pyrimidine (0.72 g, 3.4 mmol) in DMF (15 mL) was cooled in ice-water bath. DIEA (1.0 mL, 6.1 mmol) was added dropwise followed by addition of tert-butyl (1R,5S)-3,8-diazabicyclo[3.2.1]octane-8-carboxylate. The reaction was stirred at this temperature for 2 hours. Ethyl acetate (120 mL) and water (100 mL) were added. After shaking and separation, the aqueous layer was extracted with ethyl acetate (50 mL). The combined organic layers were washed with water (40 mL×3) and brine (30 mL), dried over sodium sulfate and concentrated in vacuo. The residue was purified with column chromatography (silica gel,ethyl acetate:PE=1:4) to give light brown solid (1.25 g, yield: 90%).

### Step 2: synthesis of tert-butyl (1R,5S)-3-(7-bromo-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate

In ice-water bath, to a solution of ((2R,7aS)-2-fluorotetrahydro-1H-pyrrolizin-7a(5H)-yl)methanol (0.525 g, 3.3 mmol) in anhydrous THF (30 mL) was added sodium hydride in portions (77 mg, 3.2 mmol). The reaction was stirred at room temperature for 40 min and then cooled in ice-water bath. tert-butyl (1R,5S)-3-(7-bromo-2-chloropyrido[3,2-d]pyrimidin-4-yl)-3,8-diazabicyclo[3.2.1]octane-8-carboxylate (1.25 g, 2.75 mmol) was added. The ice-water bath was removed, the stirring was kept for 6 hours. The reaction was quenched by addition of water (100 mL). Ethyl acetate (100 mL) was added. After shaking and separation, the aqueous layer was extracted with ethyl acetate (50 mL). The combined organic layers were washed with brine (30 mL), dried over sodium sulfate and concentrated in vacuo. The leaving paste was purified with column chromatography (silica gel, methanol:DCM=1:20) to give tert-butyl (1R,5S)-3-(7-bromo-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate as yellow solid (0.95 g, yield: 60%).

### Step 3: synthesis of tert-butyl (1R,5S)-3-(7-(7,8-difluoro-3-(methoxymethoxy)naphthalen-1-yl)-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate

Under argon, a mixture of tert-butyl (1R,5S)-3-(7-bromo-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate (131 mg, 0.374 mmol), 2-(7,8-difluoro-3-(methoxymethoxy)naphthalen-1-yl)-4,4,5,5-tetramethyl-1,3,2-dioxaborolane, Pd(dppf)Cl₂ (15 mg, 0.02 mmol) and potassium carbonate (58 mg, 0.42 mmol) in 1,4-dioxane (3 mL) and water (0.6 mL) was stirred at 90° C. for 4 hours. Ethyl acetate (20 mL) and water (20 mL) were added. After shaking and separation, the aqueous layer was extracted with ethyl acetate (10 mL). The combined organic layers were washed with brine (10 mL), dried over sodium sulfate and concentrated in vacuo. The residue was purified with column chromatography (silica gel, methanol:DCM=1:20) to give light brown solid (75 mg, yield: 50%). MS m/z: 721.4 [M+H]+

### Step 4: synthesis of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-5,6-difluoronaphthalen-2-ol

In ice-water bath, to a solution of tert-butyl (1R,5S)-3-(7-(7,8-difluoro-3-(methoxymethoxy) naphthalen-1-yl)-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate (75 mg, 0.104 mmol) in acetonitrile (1.5 mL) was added a solution of HCl in 1,4-dioxane (4 M, 3 mL). The reaction was stirred at this temperature for 0.5 hour. The solution was concentrated under vacuum. DCM (20 mL) and saturated aqueous saturated sodium carbonate (10 mL) were added for extraction. The organic layer was washed with water (10 mL), dried over sodium sulfate and concentrated in vacuo. The residue was purified with prep-TLC (ammonia:methanol:DCM=1:10:80) to give yellow solid (29 mg, yield: 48%). MS m/z: 577.4 [M+H]⁺. 1H NMR (400 MHz, DMSO) δ 8.21 (s, 1H), 8.08 (s, 1H), 7.66-7.56 (m, 1H), 7.46-7.35 (m, 1H), 7.34-7.31 (m, 1H), 7.23 (d, J=2.0 Hz, 1H), 5.44-5.20 (m, 1H), 4.69-4.54 (m, 2H), 4.40-4.20 (m, 2H), 3.80-3.54 (m, 4H), 3.27-3.12 (m, 3H), 3.08-2.94 (m, 1H), 2.41-2.09 (m, 3H), 2.07-1.66 (m, 7H)

The compounds of Examples 200 and 201 were prepared by the preparation method for Example

| | | | m/z: |
|---|---|---|---|
| Ex. | Compound name | Structural formula | ES⁺[M + H] |
| 200 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)pyrido[3,2-d]pyrimidin-7-yl)-5-ethynyl-6- fluoronaphthalen-2-ol | 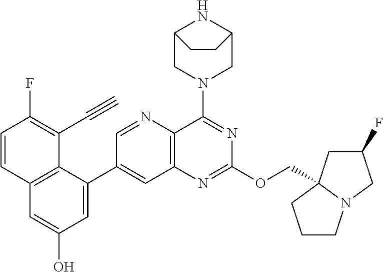 [SMILES: C#Cc1c(F)ccc2cc(O)cc(-c3cnc4c(N5CC6CCC(C5)N6)nc(OC[C@@]56CCCN5C[C@H](F)C6)nc4c3)c12] | 583.4 |
| 201 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)pyrido[3,2-d]pyrimidin-7-yl)-5-ethyl-6- fluoronaphthalen-2-ol | 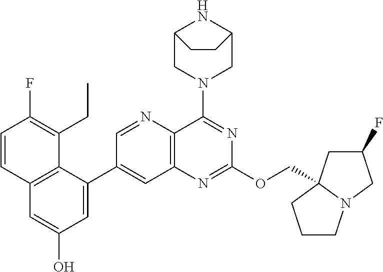 [SMILES: CCc1c(F)ccc2cc(O)cc(-c3cnc4c(N5CC6CCC(C5)N6)nc(OC[C@@]56CCCN5C[C@H](F)C6)nc4c3)c12] | 587.4 |

### Example 202: synthesis of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethynyl-6-fluoronaphthalen-2-ol

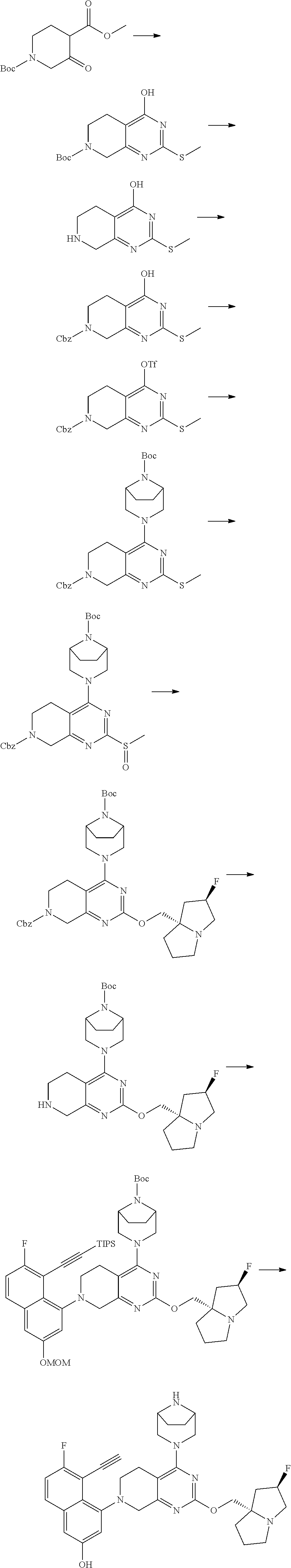

### Step 1: synthesis of tert-butyl 4-hydroxy-2-(methylthio)-5,8-dihydropyrido[3,4-d]pyrimidine-7(6H)-carboxylate

1-(tert-butyl) 4-methyl 3-oxopiperidine-1,4-dicarboxylate (20 g, 77.73 mmol) was dissolved in an aqueous solution (160 mL). 30% sodium methoxide solution (42.0 g, 233.20 mmol) and thiourea (8.88 g, 116.60 mmol) were added, the reaction was heated at 80° C. for 2 hours. After completion, the reaction was cooled to room temperature, iodomethane was added dropwise (13.79 g, 6.10 mL, 97.17 mmol) to the reaction solution. The reaction was stirred for 1 hour at room temperature. After completion, concentrate the reaction solution, dissolve the residue in water, adjust the pH to 6-7 with glacial acetic acid. Solid precipitated. Filtering gave white solid. (22 g, yield: 95%). 1H NMR (400 MHz, CDCl3) δ 11.40 (s, 1H), 4.33 (s, 2H), 3.60 (t, J=5.5 Hz, 2H), 2.56 (s, 5H), 1.49 (s, 9H).

### Step 2: synthesis of 2-(methylthio)-5,6,7,8-tetrahydropyrido[3,4-d]pyrimidin-4-ol

tert-butyl 4-hydroxy-2-(methylthio)-5,8-dihydropyrido[3,4-d]pyrimidine-7(6H)-carboxylate (22 g, 73.98 mmol) was dissolved in DCM (200 mL). TFA (50 mL) was added. The reaction was stirred at room temperature for 3 hours. After completion, the reaction mixture was concentrated. The leaving residue was extracted with saturated aqueous sodium bicarbonate and DCM. The organic layer was washed with brine, dried over sodium sulfate and concentrated to give crude product which was used directly in next step (14.6 g, yield: 100%).

### Step 3: synthesis of benzyl 4-hydroxy-2-(methylthio)-5,8-dihydropyrido[3,4-d]pyrimidine-7(6H)-carboxylate

2-(methylthio)-5,6,7,8-tetrahydropyrido[3,4-d]pyrimidin-4-ol (14.5 g, 73.51 mmol) was dissolved in anhydrous THF (150 mL). With ice-water bath cooling, TEA (15.37 mL, 110.26 mmol) was added, following by adding benzyl chloroformate (13.79 g, 80.86 mmol) dropwise. The reaction was stirred in ice-water bath for 1 hour. After completion, water was added and the mixture was extracted with ethyl acetate. The organic layer was washed with brine, dried over sodium sulfate and concentrated. Column chromatography purification gave target compound (24 g, yield: 98.5%).

### Step 4: synthesis of benzyl 2-(methylthio)-4-(((trifluoromethyl)sulfonyl)oxy)-5,8-dihydropyrido[3,4-d]pyrimidine-7(6H)-carboxylate

benzyl 4-hydroxy-2-(methylthio)-5,8-dihydropyrido[3,4-d]pyrimidine-7(6H)-carboxylate (24.0 g, 72.42 mmol) was dissolved in DCM (300 mL). With ice-water bath cooling, DIEA (14.04 g, 108.63 mmol) was added following by adding trifluoromethanesulfonic anhydride (22.48 g, 79.66 mol) dropwise. After addition, the reaction was stirred in ice-water bath for 1 hour. After completion, the reaction mixture was concentrated and the resulting oil was used directly in next step.

### Step 5: synthesis of benzyl 4-((1R,5S)-8-(tert-butoxycarbonyl)-3,8-diazabicyclo[3.2.1]octan-3-yl)-2-(methylthio)-5,8-dihydropyrido[3,4-d]pyrimidine-7(6H)-carboxylate

benzyl 2-(methylthio)-4-(((trifluoromethyl)sulfonyl)oxy)-5,8-dihydropyrido[3,4-d]pyrimidine-7(6H)-carboxylate (72.42 mmol) was dissolved in anhydrous DMF (300 mL). DIEA (14.04 g, 108.63 mmol) and tert-butyl (1R,5S)-3,8-diazabicyclo[3.2.1]octane-8-carboxylate (15.38 g, 72.42 mmol) were added. The reaction was stirred in ice-water bath for 30 min. Brine was added and the mixture was extracted with ethyl acetate. The organic layer was washed with brine, dried over sodium sulfate and concentrated. Purification by column chromatography gave off-white solid (26.6 g, yield: 70%). 1H NMR (400 MHz, CDCl3) δ 7.36 (d, J=9.9 Hz, 5H), 5.17 (s, 2H), 4.54 (s, 2H), 4.29 (s, 2H), 3.78 (s, 2H), 3.61 (d, J=4.8 Hz, 2H), 3.21 (s, 2H), 2.62 (s, 2H), 2.49 (s, 3H), 1.85 (dd, J=43.2, 5.8 Hz, 4H), 1.49 (s, 9H).

### Step 6: synthesis of benzyl 4-((1R,5S)-8-(tert-butoxycarbonyl)-3,8-diazabicyclo[3.2.1]octan-3-yl)-2-(methylsulfinyl)-5,8-dihydropyrido[3,4-d]pyrimidine-7(6H)-carboxylate

benzyl 4-((1R,5S)-8-(tert-butoxycarbonyl)-3,8-diazabicyclo[3.2.1]octan-3-yl)-2-(methylthio)-5,8-dihydropyrido[3,4-d]pyrimidine-7(6H)-carboxylate (26.6 g, 50.60 mmol) was dissolved in DCM (200 mL). With ice-water bath cooling, 85% m-CPBA (12.32 g, 60.72 mmol) was added. The reaction was stirred in ice-water bath for 30 min. After completion, saturated aqueous sodium thiosulfate was added to quench the reaction. Use DCM to run extraction, the organic layer was washed with aqueous sodium bicarbonate and brine, dried over sodium sulfate and off-white solid (27.4 g, yield: 100%). 1H NMR (400 MHz, CDCl3) δ 7.37 (s, 5H), 5.19 (s, 2H), 4.71 (s, 2H), 4.31 (s, 2H), 3.92 (s, 2H), 3.65 (s, 2H), 3.30-3.27 (m, 2H), 2.88 (s, 3H), 2.72 (s, 2H), 2.02-1.85 (m, 2H), 1.75 (d, J=7.4 Hz, 2H), 1.49 (s, 9H).

### Step 7: synthesis of benzyl 4-((1R,5S)-8-(tert-butoxycarbonyl)-3,8-diazabicyclo[3.2.1]octan-3-yl)-2-(((2R,7aS)-2-fluorotetrahydro-1H-pyrrolizin-7a(5H)-yl)methoxy)-5,8-dihydropyrido[3,4-d]pyrimidine-7(6H)-carboxylate

benzyl 4-((1R,5S)-8-(tert-butoxycarbonyl)-3,8-diazabicyclo[3.2.1]octan-3-yl)-2-(methylsulfinyl)-5,8-dihydropyrido[3,4-d]pyrimidine-7(6H)-carboxylate (2.0 g, 3.69 mmol) was dissolved in anhydrous toluene (20 mL). With ice-water bath cooling, ((2R,7aS)-2-fluorotetrahydro-1H-pyrrolizin-7a(5H)-yl)methanol (0.76 g, 4.80 mmol) was added. Then sodium tert-butoxide (0.53 g, 5.54 mmol) was added in portions. The reaction was stirred in ice-water bath for 20 min. After completion, the reaction was quenched by addition of water. The mixture was extracted with ethyl acetate. The organic layer was washed with brine, dried over sodium sulfate and concentrated. Purification with column chromatography gave off-white solid (1.53 g, yield: 65%). 1H NMR (300 MHz, CDCl3) δ 7.38 (s, 5H), 5.26 (d, J=38.3 Hz, 3H), 4.55 (s, 2H), 4.29 (s, 2H), 4.14-3.91 (m, 2H), 3.82 (d, J=12.8 Hz, 2H), 3.63 (s, 2H), 3.34-3.05 (m, 5H), 3.03-2.87 (m, 1H), 2.63 (s, 2H), 2.13 (s, 3H), 1.87 (ddd, J=23.6, 10.7, 6.0 Hz, 7H), 1.51 (s, 9H).

### Step 8: synthesis of tert-butyl (1R,5S)-3-(2-(((2R,7aS)-2-fluorotetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-8-carboxylate

benzyl 4-((1R,5S)-8-(tert-butoxycarbonyl)-3,8-diazabicyclo[3.2.1]octan-3-yl)-2-(((2R,7aS)-2-fluorotetrahydro-1H-pyrrolizin-7a(5H)-yl)methoxy)-5,8-dihydropyrido[3,4-d]pyrimidine-7(6H)-carboxylate (1.53 g, 2.40 mmol) was dissolved in methanol (30 mL) and NH₃/methanol (6M, 4 mL). Under nitrogen atmosphere, 10% wet Pd/C (300 mg) was added. The flask was evacuated and backfilled with hydrogen. The reaction was stirred at room temperature for 5 hours. After completion, the mixture was filtered through celite and eluted with methanol. The combined filtrates were concentrated to give off-white solid (1.1 g, yield: 91%). 1H NMR (300 MHz, CDCl3) δ 5.38-5.11 (m, 1H), 4.29 (s, 2H), 4.06 (t, J=8.7 Hz, 1H), 3.94 (t, J=5.0 Hz, 2H), 3.85 (d, J=12.1 Hz, 2H), 3.31-3.09 (m, 5H), 3.09-2.91 (m, 3H), 2.69-2.51 (m, 2H), 2.37-2.06 (m, 3H), 1.89 (dt, J=11.8, 8.1 Hz, 8H), 1.51 (s, 9H).

### Step 9: synthesis of tert-butyl (1R,5S)-3-(7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-8-carboxylate

tert-butyl (1R,5S)-3-(2-(((2R,7aS)-2-fluorotetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-8-carboxylate (50 mg, 0.10 mmol) and 7-fluoro-3-(methoxymethoxy)-8-((triisopropylsilyl)ethynyl)naphthalen-1-yl trifluoromethanesulfonate (46 mg, 0.12 mmol) were dissolved in anhydrous toluene (5 mL). Cesium carbonate (93 mg, 0.25 mmol), RuPhos (5 mg, 0.01 mmol) and Pd2(dba)3 (10 mg, 0.0 mmol) were added. Run evacuating and backfilling with nitrogen for three times. The reaction was heated at 100° C. for 12 hours. After completion, the reaction was cooled to room temperature and diluted with EA. The mixture was filtered and the filtrate was concentrated. The residue was purified with column chromatography to give off-white solid (30 mg, yield: 34%).

### Step 10: synthesis of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethynyl-6-fluoronaphthalen-2-ol

tert-butyl (1R,5S)-3-(7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-8-carboxylate (30 mg, 0.06 mmol) was dissolved in DMF (3 mL). CsF (42 mg, 0.28 mmol) was added. The reaction was stirred at room temperature for 2 hours. After completion, water was added and the mixture was extracted with ethyl acetate. The organic layer was washed with brine, dried over sodium sulfate and concentrated to give crude product. This product was dissolved in acetonitrile (2 mL). Then 4M HCl in 1,4-dioxane (1 mL) was added. The reaction was stirred at room temperature for 30 min. After completion, saturated aqueous sodium bicarbonate was added carefully to adjust pH to 7. The mixture was extracted with DCM. The organic layer was dried over sodium sulfate and concentrated. Purification with prep-TLC (DCM/MeOH(7N NH₃)=10/1) gave light yellow solid (20 mg, yield: 60%). MS m/z: 587.6 [M+H]+

The compounds of Examples 203-211 were prepared by the preparation method for Example 202

| | | | m/z: |
|---|---|---|---|
| | | | ES⁺ |
| Ex. | Compound name | Structural formula | [M + H] |
| 203 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5-ethyl-6-fluoronaphthalen-2-ol | 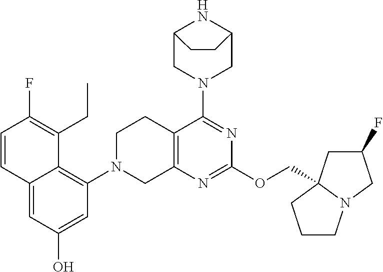 [SMILES: CCc1c(F)ccc2cc(O)cc(N3CCc4c(nc(OC[C@@]56CCCN5C[C@H](F)C6)nc4N4CC5CCC(C4)N5)C3)c12] | 591.7 |
| 204 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5,6-difluoronaphthalen-2-ol | 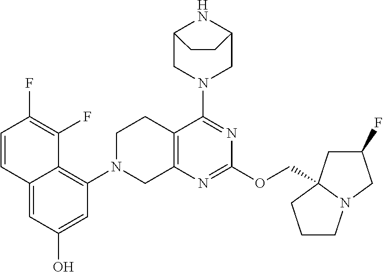 [SMILES: Oc1cc(N2CCc3c(nc(OC[C@@]45CCCN4C[C@H](F)C5)nc3N3CC4CCC(C3)N4)C2)c2c(F)c(F)ccc2c1] | 581.6 |
| 205 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5-fluoronaphthalen-2-ol | 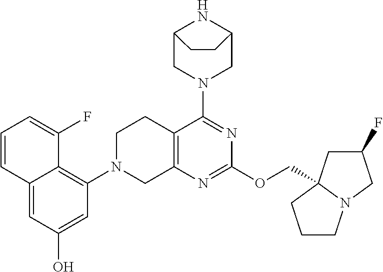 [SMILES: Oc1cc(N2CCc3c(nc(OC[C@@]45CCCN4C[C@H](F)C5)nc3N3CC4CCC(C3)N4)C2)c2c(F)cccc2c1] | 562.6 |
| 206 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5-ethynylnaphthalen-2-ol | 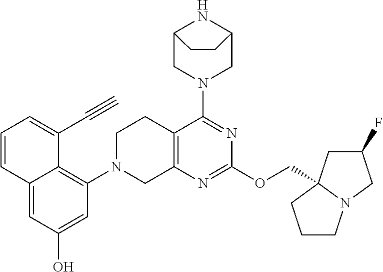 [SMILES: C#Cc1cccc2cc(O)cc(N3CCc4c(nc(OC[C@@]56CCCN5C[C@H](F)C6)nc4N4CC5CCC(C4)N5)C3)c12] | 569.7 |
| 207 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5-ethylnaphthalen-2-ol | 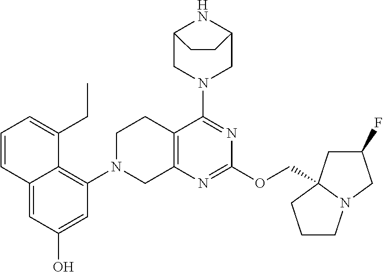 [SMILES: CCc1cccc2cc(O)cc(N3CCc4c(nc(OC[C@@]56CCCN5C[C@H](F)C6)nc4N4CC5CCC(C4)N5)C3)c12] | 573.3 |
| 208 | 4-(4-((1R,5S)-3,8-diazabicyclo[3.2.1]octan- 3-yl)-2-(((2R,7aS)-2-fluorotetrahydro-1H- pyrrolizin-7a(5H)-yl)methoxy)-5,8- dihydropyrido[3,4-d]pyrimidin- 7(6H)-yl)-5-chloro-6-fluoronaphthalen-2-ol | 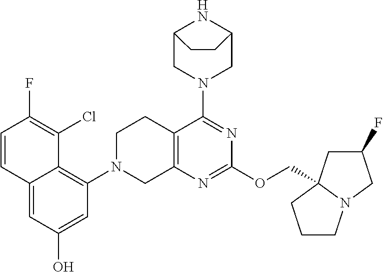 [SMILES: Oc1cc(N2CCc3c(nc(OC[C@@]45CCCN4C[C@H](F)C5)nc3N3CC4CCC(C3)N4)C2)c2c(Cl)c(F)ccc2c1] | 597 |
| 209 | ((3R,7aR)-7a-(((4-((1R,5S)-3,8- diazabicyclo[3.2.1]octan-3-yl)-7-(8- ethynyl-7-fluoro-3-hydroxynaphthalen- 1-yl)-5,6,7,8-tetrahydropyrido[3,4- d]pyrimidin-2-yl)oxy)methyl)hexahydro- 1H-pyrrolizin-3-yl)methyl dimethylcarbamate | 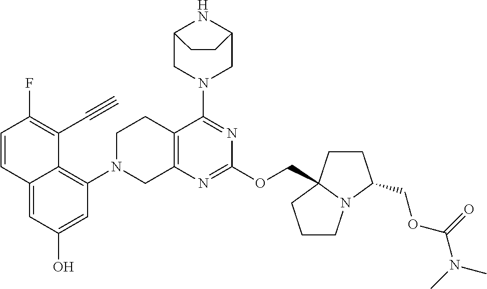 [SMILES: C#Cc1c(F)ccc2cc(O)cc(N3CCc4c(nc(OC[C@]56CCCN5[C@@H](COC(=O)N(C)C)CC6)nc4N4CC5CCC(C4)N5)C3)c12] | 670 |
| 210 | ((3R,7aR)-7a-(((4-((1R,5S)-3,8- diazabicyclo[3.2.1]octan-3-yl)-7-(8- ethyl-7-fluoro-3-hydroxynaphthalen- 1-yl)-5,6,7,8-tetrahydropyrido[3,4- d]pyrimidin-2-yl)oxy)methyl)hexahydro- 1H-pyrrolizin-3-yl)methyl dimethylcarbamate | 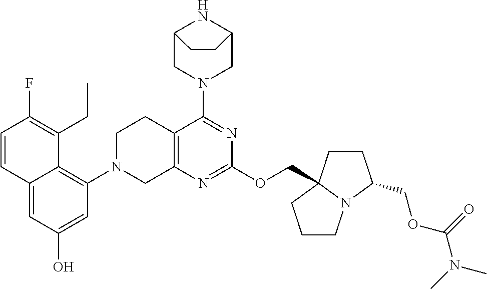 [SMILES: CCc1c(F)ccc2cc(O)cc(N3CCc4c(nc(OC[C@]56CCCN5[C@@H](COC(=O)N(C)C)CC6)nc4N4CC5CCC(C4)N5)C3)c12] | 674 |
| 211 | ((2R,7aS)-7a-(((4-((1R,5S)-3,8- diazabicyclo[3.2.1]octan-3-yl)-7-(8- ethynyl-7-fluoronaphthalen-1-yl)-5,6,7,8- tetrahydropyrido[3,4-d]pyrimidin-2- yl)oxy)methyl)hexahydro-1H- pyrrolizin-2-yl)methyl dimethylcarbamate | 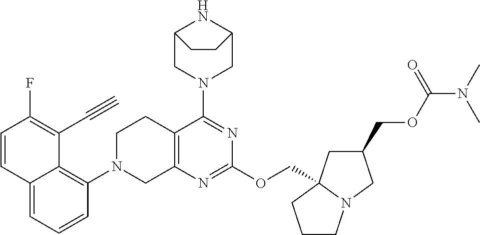 [SMILES: C#Cc1c(F)ccc2cccc(N3CCc4c(nc(OC[C@@]56CCCN5C[C@H](COC(=O)N(C)C)C6)nc4N4CC5CCC(C4)N5)C3)c12] | 654 |

### Example 212: synthesis of 4-(4-(3-(2-aminopropan-2-yl)azetidin-1-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol

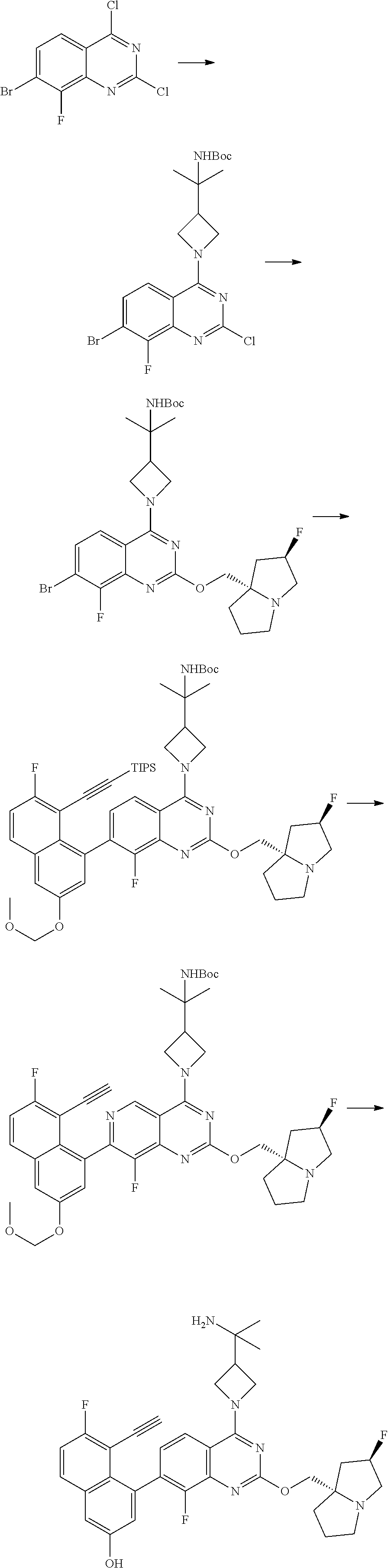

### Step 1: synthesis of tert-butyl (2-(1-(7-bromo-2-chloro-8-fluoroquinazolin-4-yl)azetidin-3-yl)propan-2-yl)carbamate

tert-butyl (2-(azetidin-3-yl)propan-2-yl)carbamate (107 mg, 0.5 mmol) and 7-bromo-2,4-dichloro-8-fluoroquinazoline (167 mg, 0.55 mmol) were dissolved in DMF (1.5 mL). DIEA (193 mg. 0.75 mmol) was added. Run evacuating and backfilling with nitrogen, the reaction was stirred at room temperature overnight. After completion, the mixture was concentrated in high vacuo and then purified with prep-TLC to give off-white solid (112 mg, yield: 47.5%). MS m/z: 473.44, 475.45 [M+H]+

### Step 2: synthesis of tert-butyl (2-(1-(7-bromo-8-fluoro-2-(((2R,7aS)-2-fluorotetrahydro-1H-pyrrolizin-7a(5H)-yl) methoxy)quinazolin-4-yl)azetidin-3-yl) propan-2-yl)carbamate

Under nitrogen atmosphere, tert-butyl (2-(1-(7-bromo-2-chloro-8-fluoroquinazolin-4-yl)azetidin-3-yl)propan-2-yl)carbamate (110 mg, 0.24 mmol) and ((2R,7aS)-2-fluorotetrahydro-1H-pyrrolizin-7a(5H)-yl)methanol (56 mg, 0.36 mmol) were dissolved in 1,4-dioxane (1 mL) and DCM (1 mL). NaH (15 mg, 0.36 mmol, 60% in mineral oil) was added at 0° C. The reaction was allowed to warm to room temperature gradually. After completion, the reaction was quenched by addition of saturated aqueous NH₄Cl (2 mL) and extracted with DCM (2 mL×3). The combined organics were washed with brine, dried over sodium sulfate and concentrated in vacuo. Purification with prep-TLC gave light yellow solid (92 mg, yield: 42.9%). MS m/z: 596.62, 598.66 [M+H]⁺.

### Step 3: synthesis of tert-butyl (2-(1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)azetidin-3-yl)propan-2-yl)carbamate

Under nitrogen atmosphere, tert-butyl (2-(1-(7-bromo-8-fluoro-2-(((2R,7aS)-2-fluorotetrahydro-1H-pyrrolizin-7a(5H)-yl)methoxy)quinazolin-4-yl)azetidin-3-yl)propan-2-yl)carbamate (92 mg, 0.15 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (118 mg, 0.23 mmol) were dissolved in 1,4-dioxane (3 mL) and water (0.8 mL). CataCXium A Pd G4 (11 mg, 0.015 mmol), potassium phosphate (97 mg, 0.45 mmol) were added. The reaction was warm up to 70° C. gradually. After completion, saturated aqueous NH₄Cl (3 mL) was added to quench the reaction. Ethyl acetate (2 mL×3) was used for extraction. The organic layer was washed with brine, dried over sodium sulfate and concentrated in vacuo. Purification with prep-TLC gave light yellow solid (77 mg, yield: 56.9%).

### Step 4: synthesis of tert-butyl (2-(1-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azetidin-3-yl)propan-2-yl)carbamate

tert-butyl (2-(1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)azetidin-3-yl)propan-2-yl)carbamate (77 mg, 0.086 mmol) was dissolved in THF (1 mL) and TBAF (0.21 mmol, 1M in THF) was added. Run evacuating and backfilling with nitrogen, the reaction was stirred at room temperature overnight. After completion, the mixture was concentrated. Purification with prep-TLC gave white solid (56 mg, yield: 91%). MS m/z: 746.96 [M+H]+

### Step 5: synthesis of 4-(4-(3-(2-aminopropan-2-yl)azetidin-1-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol

At 0° C., tert-butyl (2-(1-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azetidin-3-yl)propan-2-yl)carbamate (56 mg, 0.077 mmol) was dissolved in acetonitrile (1 mL). HCl (1 mL, 4M in 1,4-dioxane) was added. The mixture was allowed to warm to room temperature and stirred overnight. After completion, the mixture was concentrated. HCl was neutralized with aqueous sodium bicarbonate. The mixture was extracted with ethyl acetate (2 mL×3). The combined organics were washed with brine, dried over sodium sulfate and concentrated. Purification with prep-TLC gave white solid (38 mg, yield: 81.9%). MS m/z: 602.79 [M+H]+

The compounds of Examples 213-219 were prepared by the preparation method for Example 212

| | | | m/z: |
|---|---|---|---|
| Ex. | Compound name | Structural formula | ES⁺[M + H] |
| 213 | 4-(4-(3-(1-aminoethyl)azetidin-1-yl)- 8-fluoro-2-(((2R,7aS)-2- fluorotetrahydro-1H-pyrrolizin- 7a(5H)-yl)methoxy)quinazolin-7-yl)-5- ethynyl-6-fluoronaphthalen-2-ol | 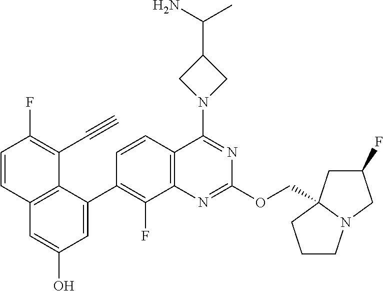 [SMILES: C#Cc1c(F)ccc2cc(O)cc(-c3ccc4c(N5CC(C(C)N)C5)nc(OC[C@@]56CCCN5C[C@H](F)C6)nc4c3F)c12] | 588.70 |
| 214 | 3-(7-(8-ethynyl-7-fluoro-3- hydroxynaphthalen-1-yl)-8- fluoro-2-(((2R,7aS)-2- fluorotetrahydro-1H-pyrrolizin- 7a(5H)-yl)methoxy)quinazolin- 4-yl)-3,8-diazabicyclo[3.2.1]octan-2-one | 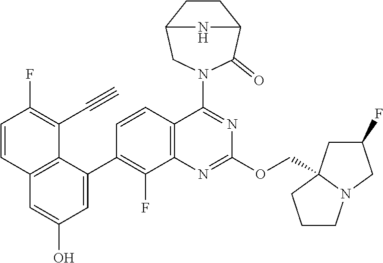 [SMILES: C#Cc1c(F)ccc2cc(O)cc(-c3ccc4c(N5CC6CCC(N6)C5=O)nc(OC[C@@]56CCCN5C[C@H](F)C6)nc4c3F)c12] | 614.67 |
| 215 | 1-(7-(8-ethynyl-7-fluoro-3- hydroxynaphthalen-1-yl)-8- fluoro-2-(((2R,7aS)-2- fluorotetrahydro-1H- pyrrolizin-7a(5H)- yl)methoxy)quinazolin- 4-yl)azetidine-3-carboxamide | 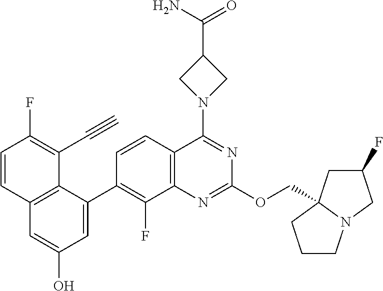 [SMILES: C#Cc1c(F)ccc2cc(O)cc(-c3ccc4c(N5CC(C(N)=O)C5)nc(OC[C@@]56CCCN5C[C@H](F)C6)nc4c3F)c12] | 588.3 |
| 216 | 5-ethynyl-6-fluoro-4-(8-fluoro-2- (((2R,7aS)-2-fluorotetrahydro- 1H-pyrrolizin-7a(5H)- yl)methoxy)-4-(1,6- diazaspiro[3.3]heptan- 6-yl)quinazolin-7- yl)naphthalen-2-ol | 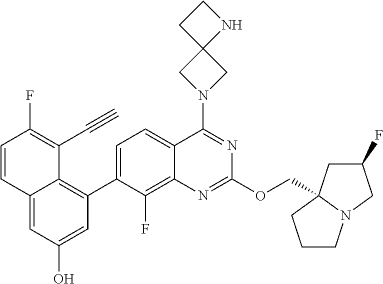 [SMILES: C#Cc1c(F)ccc2cc(O)cc(-c3ccc4c(N5CC6(CCN6)C5)nc(OC[C@@]56CCCN5C[C@H](F)C6)nc4c3F)c12] | 586.4 |
| 217 | 4-(4-(3-amino-3-methylazetidin- 1-yl)-8-fluoro-2-(((2R,7aS)-2- fluorotetrahydro-1H- pyrrolizin-7a(5H)- yl)methoxy)quinazolin-7- yl)-5-ethynyl-6- fluoronaphthalen-2-ol | 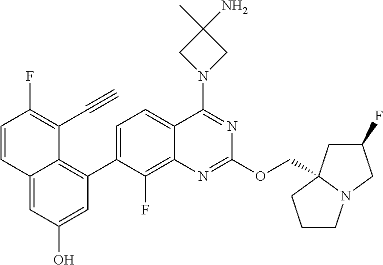 [SMILES: C#Cc1c(F)ccc2cc(O)cc(-c3ccc4c(N5CC(C)(N)C5)nc(OC[C@@]56CCCN5C[C@H](F)C6)nc4c3F)c12] | 574.4 |
| 218 | 5-ethynyl-6-fluoro-4-(8-fluoro-2- (((2R,7aS)-2-fluorotetrahydro- 1H-pyrrolizin-7a(5H)-yl)methoxy)-4- (3-methyl-3-(methylamino)azetidin- 1-yl)quinazolin-7- yl)naphthalen-2-ol | 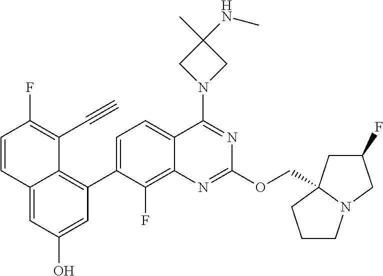 [SMILES: C#Cc1c(F)ccc2cc(O)cc(-c3ccc4c(N5CC(C)(NC)C5)nc(OC[C@@]56CCCN5C[C@H](F)C6)nc4c3F)c12] | 588.4 |
| 219 | 4-(4-(cyclopropyl(2-(methyl- amino)ethyl)amino)-8-fluoro-2- (((2R,7aS)-2-fluorotetrahydro-1H- pyrrolizin-7a(5H)- yl)methoxy)pyrido[4,3-d]pyrimidin- 7-yl)-5-ethynyl-6-fluoronaphthalen- 2-ol | 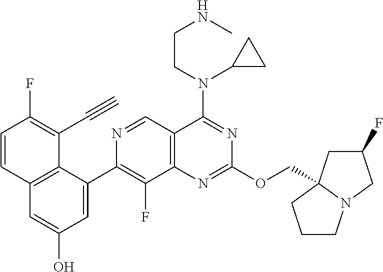 [SMILES: C#Cc1c(F)ccc2cc(O)cc(-c3ncc4c(N(CCNC)C5CC5)nc(OC[C@@]56CCCN5C[C@H](F)C6)nc4c3F)c12] | 603.4 |

### Example 220: synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethyl-2,3-dihydro-1H-inden-5-ol

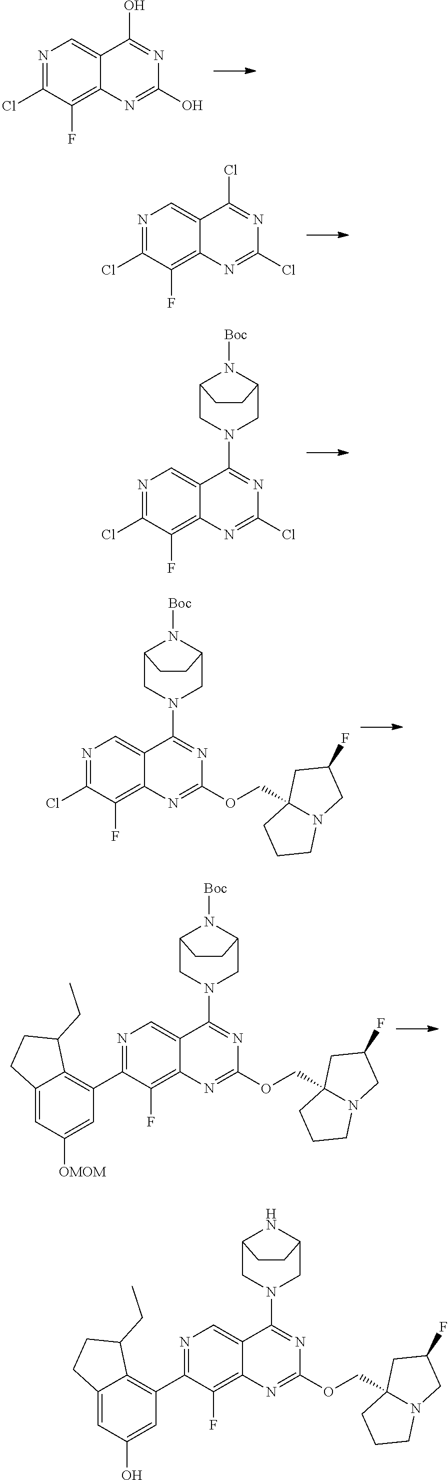

### Step 1: synthesis of 2,4,7-trichloro-8-fluoropyrido[4,3-d]pyrimidine

7-chloro-8-fluoropyrido[4,3-d]pyrimidine-2,4-diol (5.0 g, 23.1 mmol) was dissolved in 50 mL of POCl₃. 5 mL of N,N-diethyl aniline was added at room temperature. The reaction was heated at 110° C. at stirred overnight. After completion, the reaction mixture was treated with vacuum to remove organic solvents. The leaving crude product was purified with column chromatography (DCM) to give off-white solid (5.28 g, yield: 91%). MS m/z: 252[M+H]+

### Step 2: synthesis of tert-butyl (1R,5S)-3-(2,7-dichloro-8-fluoropyrido[4,3-d]pyrimidin-4-yl)-3,8-diazabicyclo[3.2.1]octane-8-carboxylate

2,4,7-trichloro-8-fluoropyrido[4,3-d]pyrimidine (2.5 g, 10 mmol) was dissolved in 50 mL of anhydrous THF, followed by addition of DIEA (2.8 ml, 20 mmol). In ice-water bath, tert-butyl (1R,5S)-3,8-diazabicyclo[3.2.1]octane-8-carboxylate (2.23 g, 10.5 mmol) was added. The reaction was stirred for 2 h. After completion, solvent was removed under vacuum to give crude product. This crude product was dissolved in 200 mL of ethyl acetate. The solution was washed with 50 mL of brine. The organic layer was dried over sodium sulfate and concentrated. The product was triturated in PE (200 mL) for 1 h. Filtering gave white solid (4.0 g, yield: 93%). MS m/z: 428[M+H]+

### Step 3: synthesis of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

((2R,7aS)-2-fluorotetrahydro-1H-pyrrolizin-7a(5H)-yl)methanol (482 mg, 3.03 mmol) was dissolved in anhydrous THF (10 mL). With ice-water bath cooling, 60% NaH (51 mg, 1.27 mmol) was added. The reaction was stirred in ice-water bath for 30 min. tert-butyl (1R,5S)-3-(2,7-dichloro-8-fluoropyrido[4,3-d]pyrimidin-4-yl)-3,8-diazabicyclo[3.2.1]octane-8-carboxylate (1.0 g, 2.33 mmol) was added. The stirred was kept overnight. After completion, the reaction was quenched by addition of cooled water. The mixture was extracted with ethyl acetate. The organic layer was washed with brine, dried over sodium sulfate and concentrated. Purification with column chromatography gave off-white solid (700 mg, yield: 55%).

### Step 4: synthesis of tert-butyl (1R,5S)-3-(7-(3-ethyl-6-(methoxymethoxy)-2,3-dihydro-1H-inden-4-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (45 mg, 0.13 mmol), 2-(3-ethyl-6-(methoxymethoxy)-2,3-dihydro-1H-inden-4-yl)-4,4,5,5-tetramethyl-1,3,2-dioxaborolane (55 mg, 0.1 mmol) and Cs₂CO₃ (65.16 mg, 0.2 mmol) were dissolved in 5 mL/1 mL of 1,4-dioxane/water. After evacuation and backfilling with argon, Pd(PPh₃)₄ (57.18 mg, 0.05 mmol) was added. After further evacuation and backfilling with argon, the reaction was heated at 100° C. overnight. After completion, solvent was removed in vacuo to give crude product. This crude product was purified with prep-TLC to give target compound as light yellow solid (59 mg, yield: 82%). MS m/z: 721[M+H]+

### Step 5: synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethyl-2,3-dihydro-1H-inden-5-ol

tert-butyl (1R,5S)-3-(7-(3-ethyl-6-(methoxymethoxy)-2,3-dihydro-1H-inden-4-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate was dissolved in 5 mL of HCl/dioxane (4M/L). The reaction was stirred at room temperature for 2 h. After completion, the reaction mixture was treated with vacuum to remove organic solvents. 100 mL of DCM was added to dissolve the residue. The solution was neutralized and washed with 10 mL of saturated aqueous sodium bicarbonate. The organic layer was dried over sodium sulfate and concentrated to give crude product. This crude product was purified with prep-TLC to give target compound as off-white solid (35 mg, yield: 48%). MS m/z: 577 [M+H]⁺. 1H NMR (400 MHz, DMSO) δ 9.31 (s, 1H), 9.07 (s, 1H), 7.24 (d, J=8.8 Hz, 1H), 6.87 (d, J=8.9 Hz, 1H), 6.72 (d, J=2.3 Hz, 1H), 6.67 (d, J=1.8 Hz, 1H), 5.33 (d, J=9.0 Hz, 1H), 5.21 (s, 1H), 4.49 (d, J=12.3 Hz, 2H), 4.27 (d, J=12.1 Hz, 1H), 4.11 (d, J=10.4 Hz, 1H), 4.02 (d, J=10.4 Hz, 1H), 3.65 (d, J=12.1 Hz, 1H), 3.50 (t, J=14.2 Hz, 1H), 3.16-3.05 (m, 1H), 3.01 (s, 1H), 2.94-2.72 (m, 3H), 2.24-1.92 (m, 4H), 1.93-1.71 (m, 3H), 1.63 (s, 3H), 1.24 (s, 2H), 1.11-0.92 (m, 3H), 0.53 (t, J=7.3 Hz, 3H).

| Ex. | Compound name | Structural formula | m/z: ES⁺[M + H] |
|---|---|---|---|
| 221 | 1^{st} eluting isomer (R)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol or (S)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol | 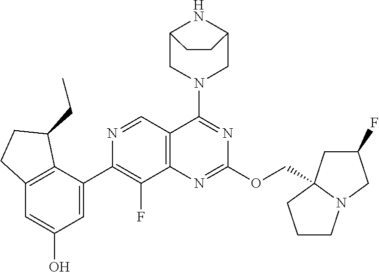 [SMILES: CC[C@@H]1CCc2cc(O)cc(-c3ncc4c(N5CC6CCC(C5)N6)nc(OC[C@@]56CCCN5C[C@H](F)C6)nc4c3F)c21] | 577.2 Column: Chiralcel OJ-3, 150 × 4.6 mm I.D., 3 um Mobile phase: A: CO2/B: IPA (0.1% IPAm, v/v Flow rate 2.5 mL/min Column temp .: 35° C. ABPR: 2000 psi |
| | | or | |
| | | 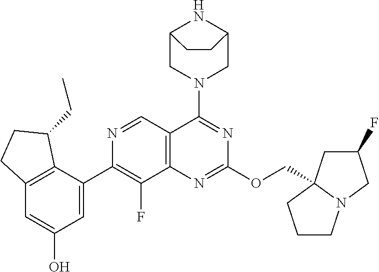 [SMILES: CC[C@H]1CCc2cc(O)cc(-c3ncc4c(N5CC6CCC(C5)N6)nc(OC[C@@]56CCCN5C[C@H](F)C6)nc4c3F)c21] | |
| 222 | 2^{nd} eluting isomer, (S)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol or (R)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol | 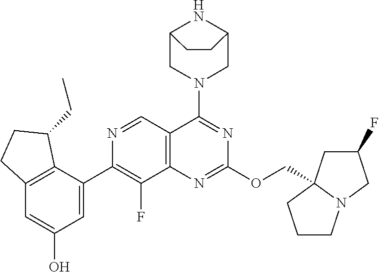 [SMILES: CC[C@H]1CCc2cc(O)cc(-c3ncc4c(N5CC6CCC(C5)N6)nc(OC[C@@]56CCCN5C[C@H](F)C6)nc4c3F)c21] | 577.2 Column: Chiralcel OJ-3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| | | or | |
| | | 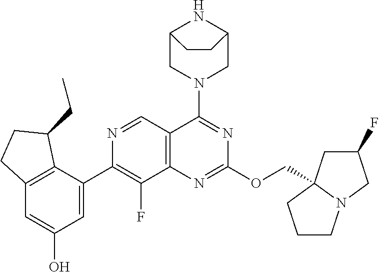 [SMILES: CC[C@@H]1CCc2cc(O)cc(-c3ncc4c(N5CC6CCC(C5)N6)nc(OC[C@@]56CCCN5C[C@H](F)C6)nc4c3F)c21] | |
| 223 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-methyl-2,3-dihydro-1H- inden-5-ol | 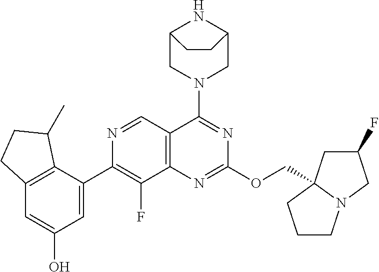 [SMILES: CC1CCc2cc(O)cc(-c3ncc4c(N5CC6CCC(C5)N6)nc(OC[C@@]56CCCN5C[C@H](F)C6)nc4c3F)c21] | 563.4 |
| 224 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-methyl-5,6,7,8- tetrahydronaphthalen-2-ol | 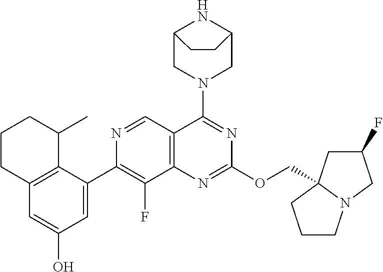 [SMILES: CC1CCCc2cc(O)cc(-c3ncc4c(N5CC6CCC(C5)N6)nc(OC[C@@]56CCCN5C[C@H](F)C6)nc4c3F)c21] | 577.2 |
| 225 | 1^{st} eluting isomer (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol or (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol | 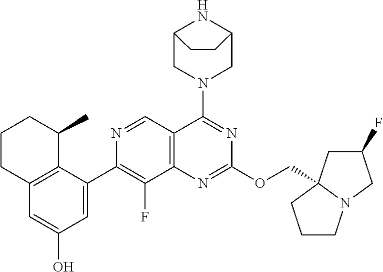 [SMILES: C[C@@H]1CCCc2cc(O)cc(-c3ncc4c(N5CC6CCC(C5)N6)nc(OC[C@@]56CCCN5C[C@H](F)C6)nc4c3F)c21] | 577.2 Column: Chiralcel OJ-3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| | | or | |
| | | 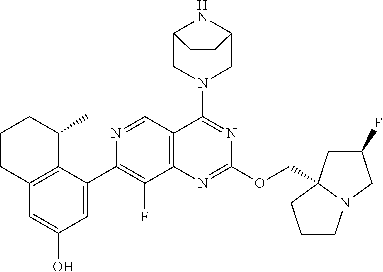 [SMILES: C[C@H]1CCCc2cc(O)cc(-c3ncc4c(N5CC6CCC(C5)N6)nc(OC[C@@]56CCCN5C[C@H](F)C6)nc4c3F)c21] | |
| 226 | 2nd eluting isomer, (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol or (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol | 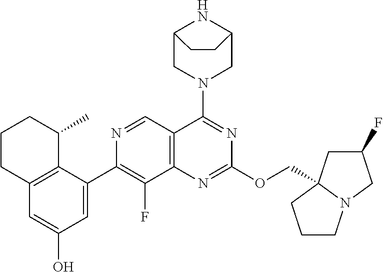 [SMILES: C[C@H]1CCCc2cc(O)cc(-c3ncc4c(N5CC6CCC(C5)N6)nc(OC[C@@]56CCCN5C[C@H](F)C6)nc4c3F)c21] | 577.2 Column: Chiralcel OJ-3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| | | or | |
| | | 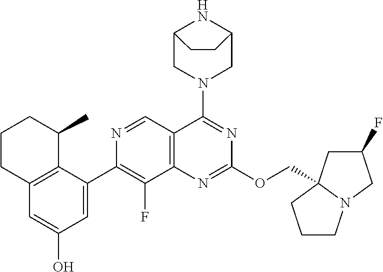 [SMILES: C[C@@H]1CCCc2cc(O)cc(-c3ncc4c(N5CC6CCC(C5)N6)nc(OC[C@@]56CCCN5C[C@H](F)C6)nc4c3F)c21] | |
| 227 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-ethyl-5,6,7,8- tetrahydronaphthalen-2-ol | 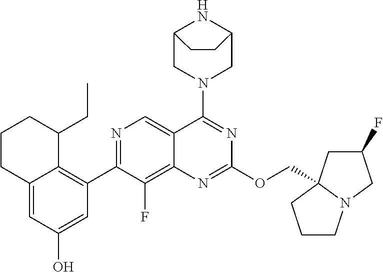 [SMILES: CCC1CCCc2cc(O)cc(-c3ncc4c(N5CC6CCC(C5)N6)nc(OC[C@@]56CCCN5C[C@H](F)C6)nc4c3F)c21] | 591.4 |
| 228 | 1^{st} eluting isomer (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol or (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol | 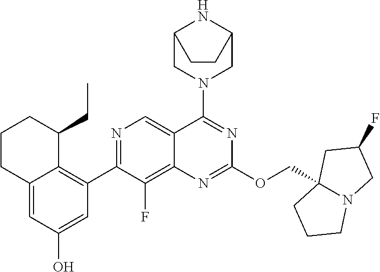 [SMILES: CC[C@@H]1CCCc2cc(O)cc(-c3ncc4c(N5CC6CCC(C5)N6)nc(OC[C@@]56CCCN5C[C@H](F)C6)nc4c3F)c21] | 591.4 Column: Chiralcel OJ-3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| | | or | |
| | | 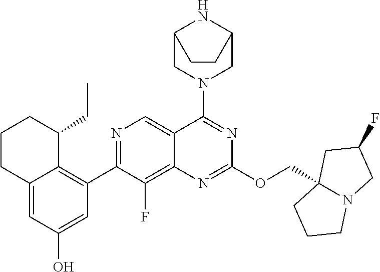 [SMILES: CC[C@H]1CCCc2cc(O)cc(-c3ncc4c(N5CC6CCC(C5)N6)nc(OC[C@@]56CCCN5C[C@H](F)C6)nc4c3F)c21] | |
| 229 | 1^{st} eluting isomer (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol or (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol | 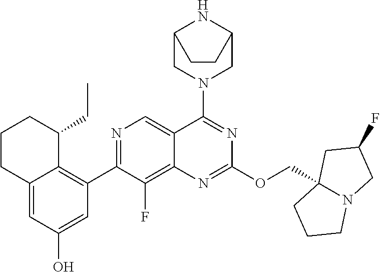 [SMILES: CC[C@H]1CCCc2cc(O)cc(-c3ncc4c(N5CC6CCC(C5)N6)nc(OC[C@@]56CCCN5C[C@H](F)C6)nc4c3F)c21] | 591.4 Column: Chiralcel OJ-3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| | | or | |
| | | 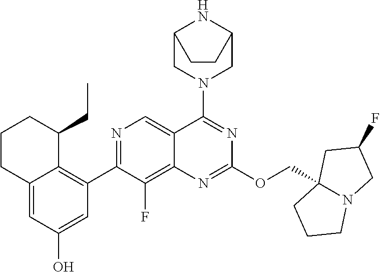 [SMILES: CC[C@@H]1CCCc2cc(O)cc(-c3ncc4c(N5CC6CCC(C5)N6)nc(OC[C@@]56CCCN5C[C@H](F)C6)nc4c3F)c21] | |
| 230 | (R)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol, S-axial chirality isomer | 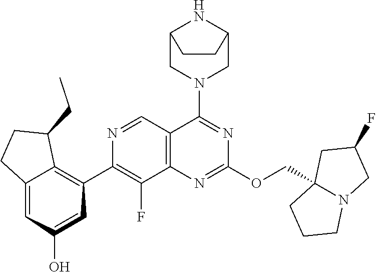 | 577.2 |
| 231 | (S)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol S-axial chirality isomer | 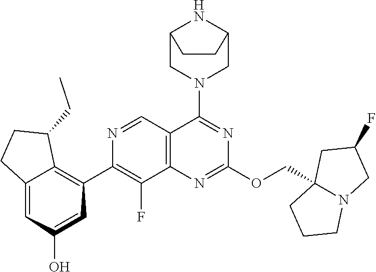 | 577.2 |
| 232 | (R)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol R-axial chirality isomer | 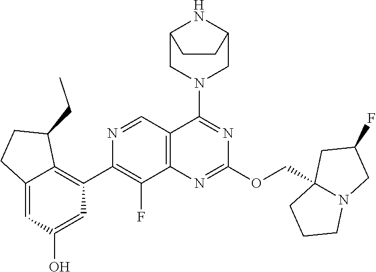 | 577.2 |
| 233 | (S)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethyl-2,3-dihydro-1H-inden-5-ol R-axial chirality isomer | 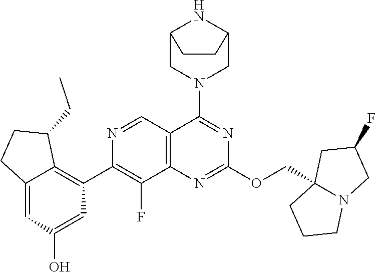 | 577.2 |
| 234 | (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol S-axial chirality isomer | 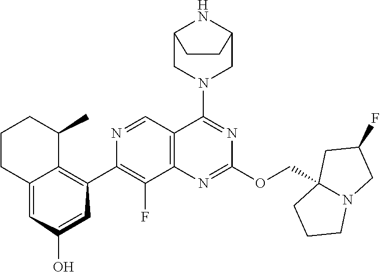 | 577.2 |
| 235 | (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol S-axial chirality isomer | 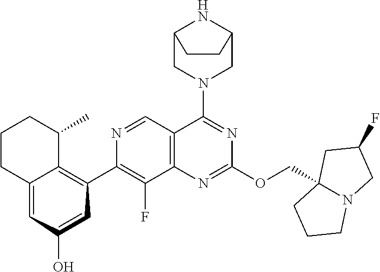 | 577.2 |
| 236 | (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol R-axial chirality isomer | 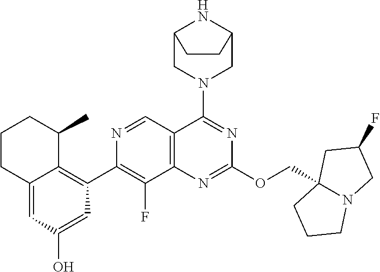 | 577.2 |
| 237 | (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-methyl-5,6,7,8-tetrahydronaphthalen-2- ol R-axial chirality isomer | 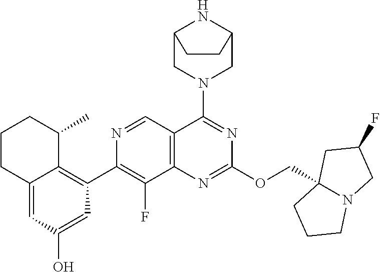 | 577.2 |
| 238 | (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol S-axial chirality isomer | 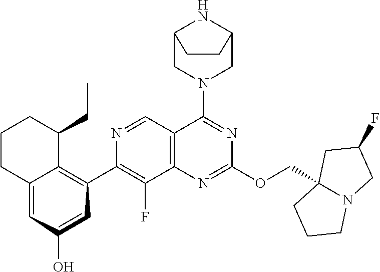 | 591.4 |
| 239 | (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol S-axial chirality isomer | 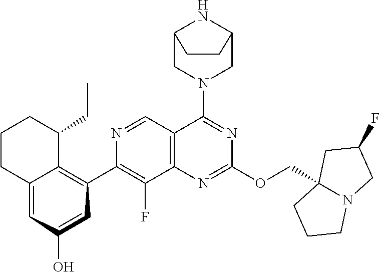 | 591.4 |
| 240 | (R)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol R-axial chirality isomer | 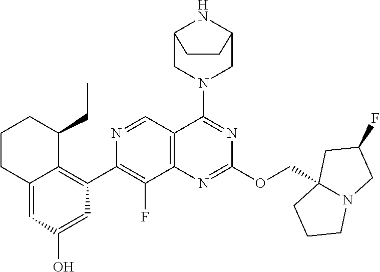 | 591.4 |
| 241 | (S)-4-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-5-ethyl-5,6,7,8-tetrahydronaphthalen-2-ol, R-axial chirality isomer | 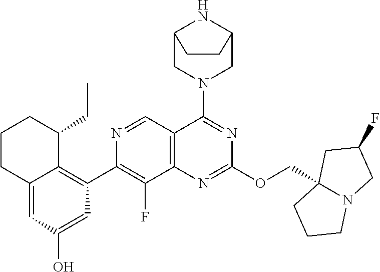 | 591.4 |
| 242 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-propyl-2,3-dihydro-1H- inden-5-ol | 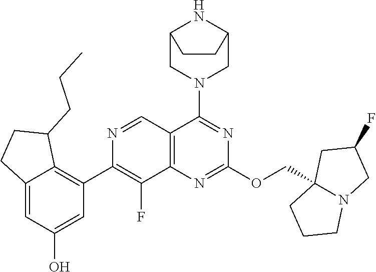 [SMILES: CCCC1CCc2cc(O)cc(-c3ncc4c(N5CC6CCC(C5)N6)nc(OC[C@@]56CCCN5C[C@H](F)C6)nc4c3F)c21] | 591.5 |
| 243 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-3-ethyl-2,3- dihydrobenzofuran-6-ol | 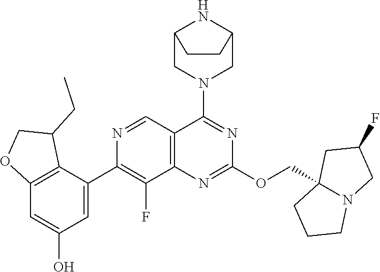 [SMILES: CCC1COc2cc(O)cc(-c3ncc4c(N5CC6CCC(C5)N6)nc(OC[C@@]56CCCN5C[C@H](F)C6)nc4c3F)c21] | 579.4 |
| 244 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-3-ethylbenzofuran-6-ol | 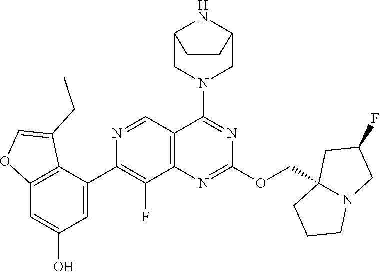 [SMILES: CCc1coc2cc(O)cc(-c3ncc4c(N5CC6CCC(C5)N6)nc(OC[C@@]56CCCN5C[C@H](F)C6)nc4c3F)c12] | 577.4 |
| 245 | 5-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1,1a,6,6a- tetrahydrocyclopropa[a]inden-3-ol | 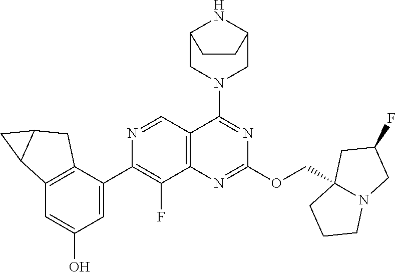 [SMILES: Oc1cc(-c2ncc3c(N4CC5CCC(C4)N5)nc(OC[C@@]45CCCN4C[C@H](F)C5)nc3c2F)c2c(c1)C1CC1C2] | 561.4 |
| 246 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1a,2,3,7b-tetrahydro-1H- cyclopropa[a]naphthalen-5-ol | 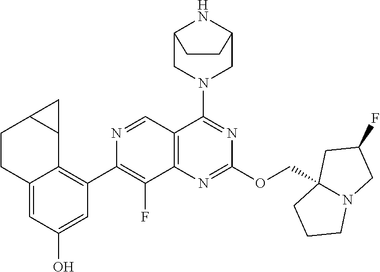 [SMILES: Oc1cc2c(c(-c3ncc4c(N5CC6CCC(C5)N6)nc(OC[C@@]56CCCN5C[C@H](F)C6)nc4c3F)c1)C1CC1CC2] | 575.4 |
| 247 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-ethynyl-2,3-dihydro-1H- inden-5-ol | 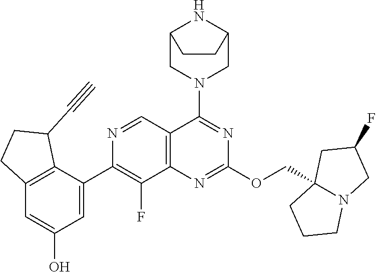 [SMILES: C#CC1CCc2cc(O)cc(-c3ncc4c(N5CC6CCC(C5)N6)nc(OC[C@@]56CCCN5C[C@H](F)C6)nc4c3F)c21] | 573.4 |
| 248 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-ethynyl-5,6,7,8- tetrahydronaphthalen-2-ol | 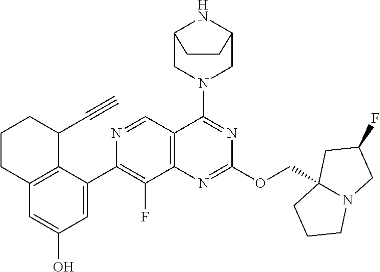 [SMILES: C#CC1CCCc2cc(O)cc(-c3ncc4c(N5CC6CCC(C5)N6)nc(OC[C@@]56CCCN5C[C@H](F)C6)nc4c3F)c21] | 587.4 |
| 249 | 8-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-6-hydroxy-3,4- dihydronaphthalen-1(2H)-one | 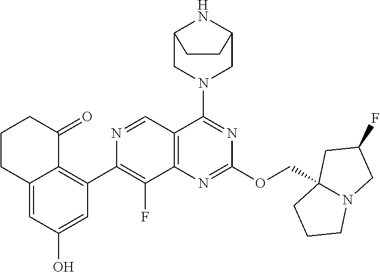 [SMILES: O=C1CCCc2cc(O)cc(-c3ncc4c(N5CC6CCC(C5)N6)nc(OC[C@@]56CCCN5C[C@H](F)C6)nc4c3F)c21] | 577.4 |
| 250 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-hydroxy-2,3-dihydro-1H- inden-1-one | 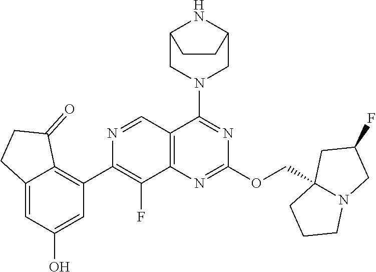 [SMILES: O=C1CCc2cc(O)cc(-c3ncc4c(N5CC6CCC(C5)N6)nc(OC[C@@]56CCCN5C[C@H](F)C6)nc4c3F)c21] | 562.4 |
| 251 | 8-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-6-hydroxythiochromane 1- oxide | 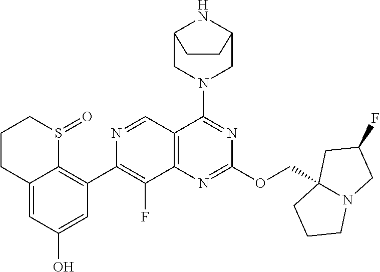 [SMILES: O=S1CCCc2cc(O)cc(-c3ncc4c(N5CC6CCC(C5)N6)nc(OC[C@@]56CCCN5C[C@H](F)C6)nc4c3F)c21] | 597.4 |
| 252 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-hydroxy-2,3- dihydrobenzo[b]thiophene 1-oxide | 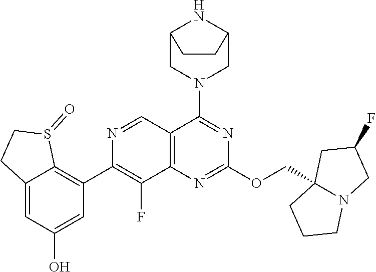 [SMILES: O=S1CCc2cc(O)cc(-c3ncc4c(N5CC6CCC(C5)N6)nc(OC[C@@]56CCCN5C[C@H](F)C6)nc4c3F)c21] | 573.3 |
| 253 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-methylene-5,6,7,8- tetrahydronaphthalen-2-ol | 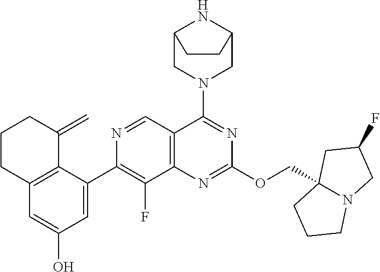 [SMILES: C=C1CCCc2cc(O)cc(-c3ncc4c(N5CC6CCC(C5)N6)nc(OC[C@@]56CCCN5C[C@H](F)C6)nc4c3F)c21] | 575.4 |
| 254 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5,5-dimethyl-5,6,7,8- tetrahydronaphthalen-2-ol | 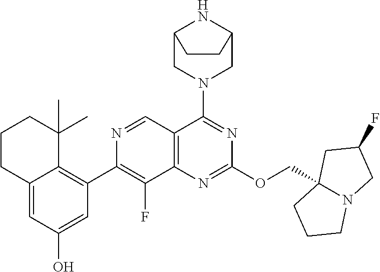 [SMILES: CC1(C)CCCc2cc(O)cc(-c3ncc4c(N5CC6CCC(C5)N6)nc(OC[C@@]56CCCN5C[C@H](F)C6)nc4c3F)c21] | 591.4 |
| 255 | 7′-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-2′,3′- dihydrospiro[cyclopropane-1,1′-inden]-5′-ol | 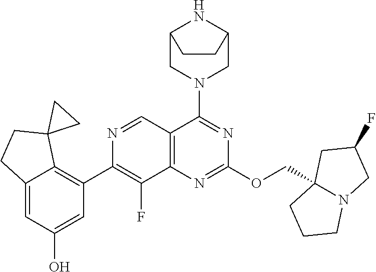 [SMILES: Oc1cc2c(c(-c3ncc4c(N5CC6CCC(C5)N6)nc(OC[C@@]56CCCN5C[C@H](F)C6)nc4c3F)c1)C1(CC2)CC1] | 575.4 |
| 256 | 8′-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-3′,4′-dihydro-2′H- spiro[cyclopropane-1,1′-naphthalen]-6′-ol | 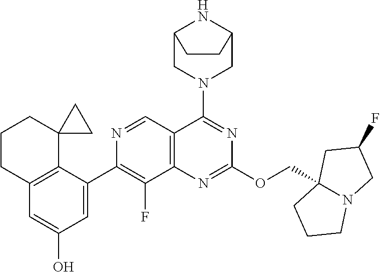 [SMILES: Oc1cc2c(c(-c3ncc4c(N5CC6CCC(C5)N6)nc(OC[C@@]56CCCN5C[C@H](F)C6)nc4c3F)c1)C1(CCC2)CC1] | 589.4 |
| 257 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-(2,2,2-trifluoroethyl)-2,3- dihydro-1H-inden-5-ol | 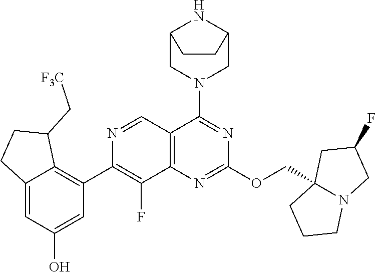 [SMILES: Oc1cc2c(c(-c3ncc4c(N5CC6CCC(C5)N6)nc(OC[C@@]56CCCN5C[C@H](F)C6)nc4c3F)c1)C(CC(F)(F)F)CC2] | 631.4 |
| 258 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-(2,2-difluoroethyl)-2,3- dihydro-1H-inden-5-ol | 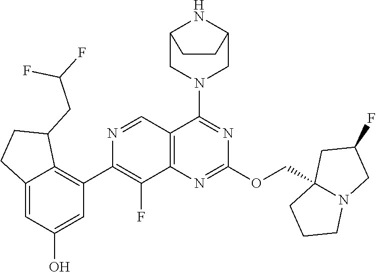 [SMILES: Oc1cc2c(c(-c3ncc4c(N5CC6CCC(C5)N6)nc(OC[C@@]56CCCN5C[C@H](F)C6)nc4c3F)c1)C(CC(F)F)CC2] | 613.4 |
| 259 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-(2-fluoroethyl)-2,3- dihydro-1H-inden-5-ol | 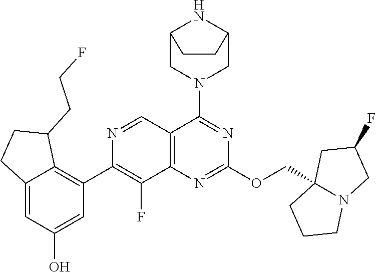 [SMILES: Oc1cc2c(c(-c3ncc4c(N5CC6CCC(C5)N6)nc(OC[C@@]56CCCN5C[C@H](F)C6)nc4c3F)c1)C(CCF)CC2] | 595.4 |
| 260 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-(trifluoromethyl)-5,6,7,8- tetrahydronaphthalen-2-ol | 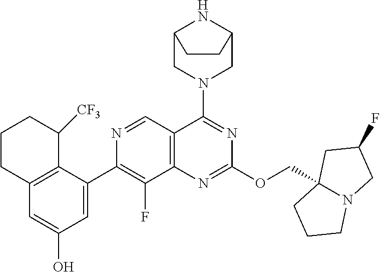 [SMILES: Oc1cc2c(c(-c3ncc4c(N5CC6CCC(C5)N6)nc(OC[C@@]56CCCN5C[C@H](F)C6)nc4c3F)c1)C(C(F)(F)F)CCC2] | 631.4 |
| 261 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-(difluoromethyl)-5,6,7,8- tetrahydronaphthalen-2-ol | 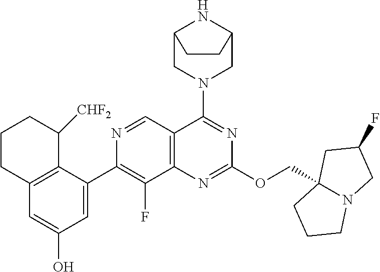 [SMILES: Oc1cc2c(c(-c3ncc4c(N5CC6CCC(C5)N6)nc(OC[C@@]56CCCN5C[C@H](F)C6)nc4c3F)c1)C(C(F)F)CCC2] | 613.4 |
| 262 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-(fluoromethyl)-5,6,7,8- tetrahydronaphthalen-2-ol | 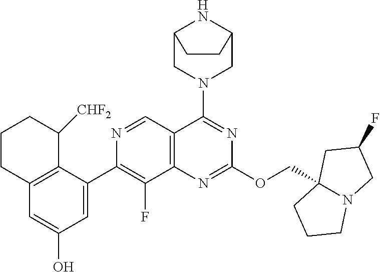 [SMILES: Oc1cc2c(c(-c3ncc4c(N5CC6CCC(C5)N6)nc(OC[C@@]56CCCN5C[C@H](F)C6)nc4c3F)c1)C(C(F)F)CCC2] | 595.4 |
| 263 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-ethyl-5-hydroxy-1,3- dihydro-2H-inden-2-one | 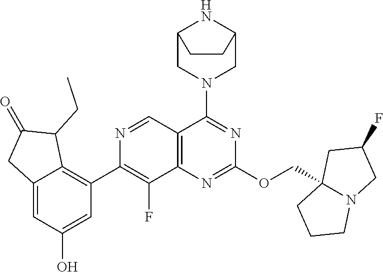 [SMILES: CCC1C(=O)Cc2cc(O)cc(-c3ncc4c(N5CC6CCC(C5)N6)nc(OC[C@@]56CCCN5C[C@H](F)C6)nc4c3F)c21] | 591.4 |
| 264 | (Z)-7-(4-((1R,5S)-3,8- diazabicyclo[3.2.1]octan-3-yl)-8-fluoro-2- (((2R,7aS)-2-fluorotetrahydro-1H-pyrrolizin- 7a(5H)-yl)methoxy)pyrido[4,3-d]pyrimidin-7- yl)-1-ethylidene-5-hydroxy-1,3-dihydro-2H- inden-2-one | 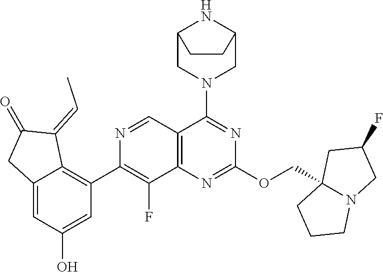 [SMILES: C/C=C1\C(=O)Cc2cc(O)cc(-c3ncc4c(N5CC6CCC(C5)N6)nc(OC[C@@]56CCCN5C[C@H](F)C6)nc4c3F)c21] | 589.4 |
| 265 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1 -(hydroxymethyl)-2,3- dihydro-1H-inden-5-ol | 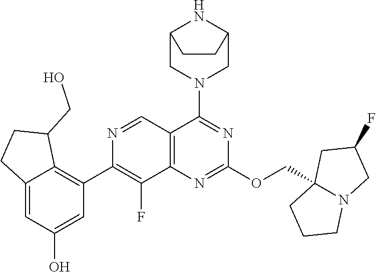 [SMILES: OCC1CCc2cc(O)cc(-c3ncc4c(N5CC6CCC(C5)N6)nc(OC[C@@]56CCCN5C[C@H](F)C6)nc4c3F)c21] | 579.4 |
| 266 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-2-methyl-2,3-dihydro-1H- inden-5-ol | 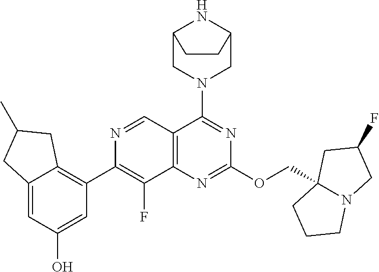 [SMILES: CC1Cc2cc(O)cc(-c3ncc4c(N5CC6CCC(C5)N6)nc(OC[C@@]56CCCN5C[C@H](F)C6)nc4c3F)c2C1] | 563.4 |
| 267 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-cyclopropyl-2,3-dihydro- 1H-inden-5-ol | 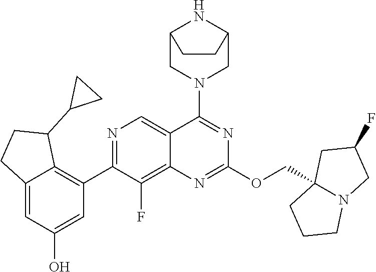 [SMILES: Oc1cc2c(c(-c3ncc4c(N5CC6CCC(C5)N6)nc(OC[C@@]56CCCN5C[C@H](F)C6)nc4c3F)c1)C(C1CC1)CC2] | 589.3 |
| 268 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-isopropyl-2,3-dihydro- 1H-inden-5-ol | 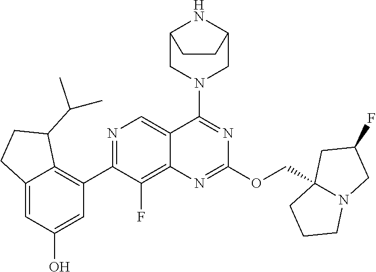 [SMILES: CC(C)C1CCc2cc(O)cc(-c3ncc4c(N5CC6CCC(C5)N6)nc(OC[C@@]56CCCN5C[C@H](F)C6)nc4c3F)c21] | 591.3 |
| 269 | 7-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-1-methoxy-2,3-dihydro-1H- inden-5-ol | 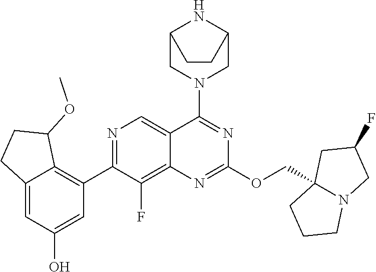 [SMILES: COC1CCc2cc(O)cc(-c3ncc4c(N5CC6CCC(C5)N6)nc(OC[C@@]56CCCN5C[C@H](F)C6)nc4c3F)c21] | 579.28 |
| 270 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-methoxy-5,6,7,8- tetrahydronaphthalen-2-ol | 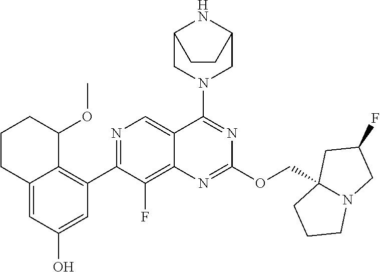 [SMILES: COC1CCCc2cc(O)cc(-c3ncc4c(N5CC6CCC(C5)N6)nc(OC[C@@]56CCCN5C[C@H](F)C6)nc4c3F)c21] | 593.4 |
| 271 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5,6,7,8- tetrahydronaphthalen-2-ol | 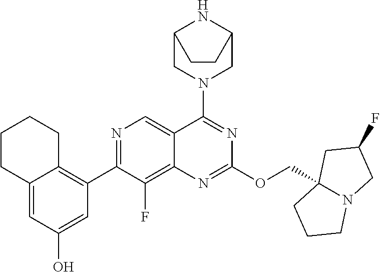 [SMILES: Oc1cc2c(c(-c3ncc4c(N5CC6CCC(C5)N6)nc(OC[C@@]56CCCN5C[C@H](F)C6)nc4c3F)c1)CCCC2] | 563.6 |
| 272 | 4-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-5-fluoro-5,6,7,8- tetrahydronaphthalen-2-ol | 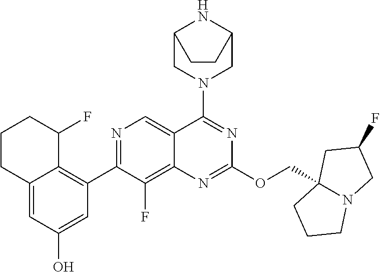 [SMILES: Oc1cc2c(c(-c3ncc4c(N5CC6CCC(C5)N6)nc(OC[C@@]56CCCN5C[C@H](F)C6)nc4c3F)c1)C(F)CCC2] | 581.4 |
| 273 | 8-(4-((1R,5S)-3,8-diazabicyclo[3.2.1]octan-3- yl)-8-fluoro-2-(((2R,7aS)-2-fluorotetrahydro- 1H-pyrrolizin-7a(5H)-yl)methoxy)pyrido[4,3- d]pyrimidin-7-yl)-6-hydroxy-2-methyl-3,4- dihydroisoquinolin-1(2H)-one | 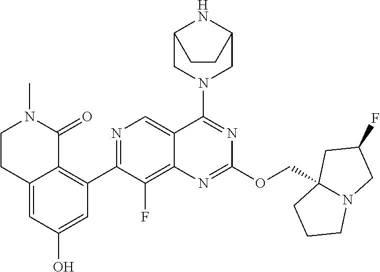 [SMILES: CN1CCc2cc(O)cc(-c3ncc4c(N5CC6CCC(C5)N6)nc(OC[C@@]56CCCN5C[C@H](F)C6)nc4c3F)c2C1=O] | 592.4 |
| 274 | 4-(4-((1R,5S)-3,8-diazabicyclo[3.2.1]octan-3- yl)-8-fluoro-2-((2-methyl-2- azabicyclo[2.2.1]heptan-1- yl)methoxy)pyrido[4,3-d]pyrimidin-7-yl)-5- ethynyl-6-fluoronaphthalen-2-ol | 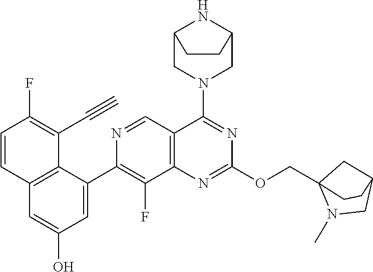 [SMILES: C#Cc1c(F)ccc2cc(O)cc(-c3ncc4c(N5CC6CCC(C5)N6)nc(OCC56CCC(CN5C)C6)nc4c3F)c12] | 583.4 |
| 275 | 4-(4-((1R,5S)-3,8-diazabicyclo[3.2.1]octan-3- yl)-8-fluoro-2-(((3R)-3-fluoro-1- azabicyclo[2.2.1]heptan-7- yl)methoxy)pyrido[4,3-d]pyrimidin-7-yl)-5- ethynyl-6-fluoronaphthalen-2-ol | 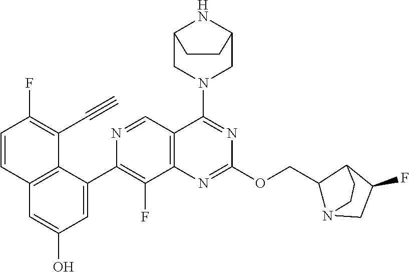 [SMILES: C#Cc1c(F)ccc2cc(O)cc(-c3ncc4c(N5CC6CCC(C5)N6)nc(OCC5C6CCN5C[C@@H]6F)nc4c3F)c12] | 587.3 |
| 276 | 4-(2-((1-azabicyclo[2.2.1]heptan-7- yl)methoxy)-4-((1R,5S)-3,8- diazabicyclo[3.2.1]octan-3-yl)-8- fluoropyrido[4,3-d]pyrimidin-7-yl)-5-ethynyl- 6-fluoronaphthalen-2-ol | 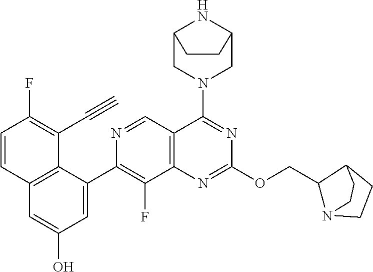 [SMILES: C#Cc1c(F)ccc2cc(O)cc(-c3ncc4c(N5CC6CCC(C5)N6)nc(OCC5C6CCN5CC6)nc4c3F)c12] | 569.4 |
| 277 | 4-(4-((1R,5S)-3,8-diazabicyclo[3.2.1]octan-3- yl)-8-fluoro-2-((6-methyl-6- azabicyclo[3.2.1]octan-5- yl)methoxy)pyrido[4,3-d]pyrimidin-7-yl)-5- ethynyl-6-fluoronaphthalen-2-ol | 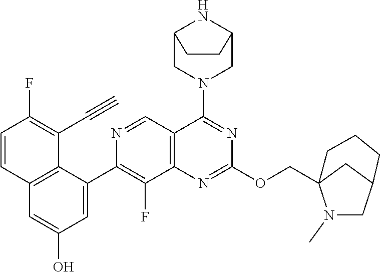 [SMILES: C#Cc1c(F)ccc2cc(O)cc(-c3ncc4c(N5CC6CCC(C5)N6)nc(OCC56CCCC(CN5C)C6)nc4c3F)c12] | 597.4 |

### Example 278: Synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethyl-1H-benzo[d]imidazol-5-ol

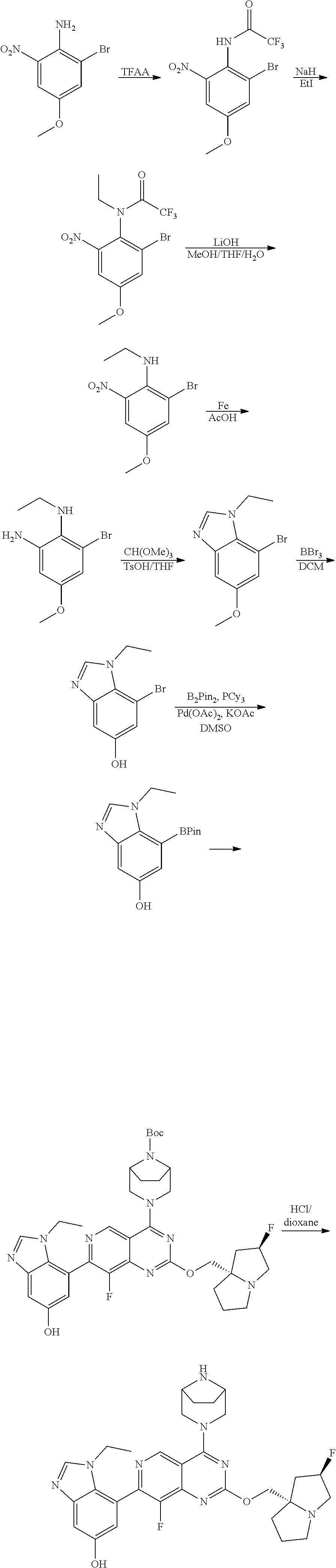

### Step 1: Synthesis of N-(2-bromo-4-methoxy-6-nitrophenyl)-2,2,2-trifluoroacetamide

2-Bromo-4-methoxy-6-nitroaniline (2 g, 8.1 mmol) in TFAA (20 mL) was stirred at 18° C. for 1 h. After the reaction was complete, the mixture was concentrated, diluted with sat. NaHCO₃ and extracted with ethyl acetate. The organic phase was washed with sat. NaCl, dried with anhydrous sodium sulfate and concentrated to obtain a yellow solid. (2.8 g, yield: 100%).

### Step 2: Synthesis of N-(2-bromo-4-methoxy-6-nitrophenyl)-N-ethyl-2,2,2-trifluoroacetamide

N-(2-bromo-4-methoxy-6-nitrophenyl)-2,2,2-trifluoroacetamide (2.8 g, 8.1 mmol) in THF (50 mL) at 0° C., was added NaH (356 mg, 8.9 mmol) portion wise. The mixture was stirred at 0° C. for 15 min. EtI (2.5 g, 16.2 mmol) was then added to the reaction liquid, the mixture was heated to 60° C. and reacted under stirring for 20 h. After the reaction was complete, the mixture was quenched by sat. NH₄Cl and extracted with ethyl acetate. The organic phase was washed with sat. NaCl, dried with anhydrous sodium sulfate, concentrated and purified by column chromatography to obtain a yellow solid. (1.7 g, yield: 56%).

### Step 3: Synthesis of 2-bromo-N-ethyl-4-methoxy-6-nitroaniline

N-(2-bromo-4-methoxy-6-nitrophenyl)-N-ethyl-2,2,2-trifluoroacetamide (1.7 g, 4.6 mmol) and LiOH·H₂O (966 mg, 23 mmol) were stirred in MeOH/H₂O/THF (10 mL/5 mL/10 mL) at room temperature for 2 h. After the reaction was complete, the mixture was extracted with ethyl acetate and sat. NaCl, concentrated to obtain a yellow solid. (1 g, yield: 79%).

### Step 4: Synthesis of 6-bromo-N1-ethyl-4-methoxybenzene-1,2-diamine

2-Bromo-N-ethyl-4-methoxy-6-nitroaniline (1 g, 3.6 mmol) and Fe powder (613 mg, 10.9 mmol) in AcOH (10 mL) were stirred at 60° C. for 2 h. After the reaction was complete, the mixture was filtered and concentrated, then dissolved in ethyl acetate and saturated sodium bicarbonate. The organic phase was washed with sat. NaCl, dried with anhydrous sodium sulfate, concentrated to obtain a yellow solid. (780 mg, yield: 88%).

### Step 5: Synthesis of 7-bromo-1-ethyl-5-methoxy-1H-benzo[d]imidazole

6-Bromo-N1-ethyl-4-methoxybenzene-1,2-diamine (780 mg, 3.2 mmol), trimethyl orthoformate (1.01 g, 9.6 mmol) and TsOH (55 mg, 0.32 mmol) in THF (10 mL) were stirred at 70° C. for 1 h. After the reaction was complete, the mixture was concentrated, then dissolved in ethyl acetate and saturated sodium bicarbonate. The organic phase was dried with anhydrous sodium sulfate, concentrated to obtain a yellow solid. (700 mg, yield: 86%).

### Step 6: Synthesis of 7-bromo-1-ethyl-1H-benzo[d]imidazol-5-ol

Compound 7-bromo-1-ethyl-5-methoxy-1H-benzo [d] imidazole (700 mg, 2.7 mmol) in anhydrous DCM (25 mL) at 0° C., was added BBr₃ (27 mL, 27 mmol, 1M), and stirred for 1 hour under ice water bath cooling. After the reaction was completed, the reaction solution was poured into saturated aqueous solution of sodium bicarbonate to quench the reaction. The mixture was extracted with dichloromethane. The organic phase was dried with anhydrous sodium sulfate, concentrated to obtain the crude product. (660 mg, yield: 100%). ¹H NMR (400 MHz, DMSO) δ 9.48 (s, 1H), 8.19 (s, 1H), 7.06-6.89 (m, 2H), 4.44 (d, J=7.1 Hz, 2H), 1.39 (t, J=7.1 Hz, 3H).

### Step 7: Synthesis of 1-ethyl-7-(4,4,5,5-tetramethyl-1,3,2-dioxaborolan-2-yl)-1H-benzo[d]imidazol-5-ol

A solution of 7-bromo-1-ethyl-1H-benzo[d]imidazol-5-ol (200 mg, 0.83 mmol), B₂Pin₂(316 mg, 1.24 mmol), KOAc (244 mg, 2.5 mmol), PCy₃ (46 mg, 0.16 mmol) and Pd(OAc)₂ (19 mg, 0.08 mmol) in DMSO (5 mL) was stirred under N₂ at 100° C. for 16h. After the reaction was complete, the mixture was extracted with ethyl acetate and H₂O. The organic phase was washed with sat. NaCl, dried with anhydrous sodium sulfate, concentrated and purified by column chromatography to obtain a brown solid. (84 mg, yield: 35%). ¹H NMR (400 MHz, DMSO) δ 9.05 (s, 1H), 8.10 (s, 1H), 7.14 (d, J=2.3 Hz, 1H), 7.07 (d, J=2.2 Hz, 1H), 4.50 (q, J=7.0 Hz, 2H), 1.35 (s, 12H), 1.28 (t, J=7.1 Hz, 3H).

### Step 8 to step 9: Synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethyl-1H-benzo[d]imidazol-5-ol

Using the method from step 4 to step 5 in the synthesis of Example 220, a yellow solid was obtained. (5 mg, two-step overall yield: 3%). ¹H NMR (400 MHz, DMSO) δ 9.12 (s, 1H), 8.33 (s, 1H), 8.14 (s, 1H), 7.11 (s, 1H), 6.80 (s, 1H), 5.35 (s, 1H), 5.22 (s, 1H), 4.41 (d, J=11.5 Hz, 2H), 4.12 (d, J=10.1 Hz, 1H), 4.02 (d, J=10.2 Hz, 1H), 3.87 (d, J=7.2 Hz, 1H), 3.72-3.50 (m, 3H), 3.05 (d, J=30.5 Hz, 2H), 2.83 (s, 1H), 2.20-1.93 (m, 2H), 1.81 (d, J=36.3 Hz, 2H), 1.60 (s, 2H), 1.23 (s, 2H), 0.92-0.71 (m, 3H).

Example 267 was obtained by using the method of Example 266.

| Ex. | Compound name | Structural formula | m/z: ES⁺ [M + H]⁺ |
|---|---|---|---|
| 279 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)pyrido[4,3- d]pyrimidin-7-yl)-1-methyl-1H-benzo[d]imidazol- 5-ol | 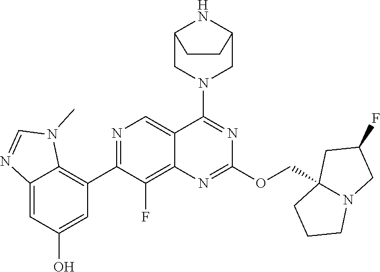 [SMILES: Cn1cnc2cc(O)cc(-c3ncc4c(N5CC6CCC(C5)N6)nc(OC[C@@]56CCCN5C[C@H](F)C6)nc4c3F)c21] | 563.4 |

### Example 280: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-methoxy-5,6,7,8-tetrahydronaphthalen-2-ol

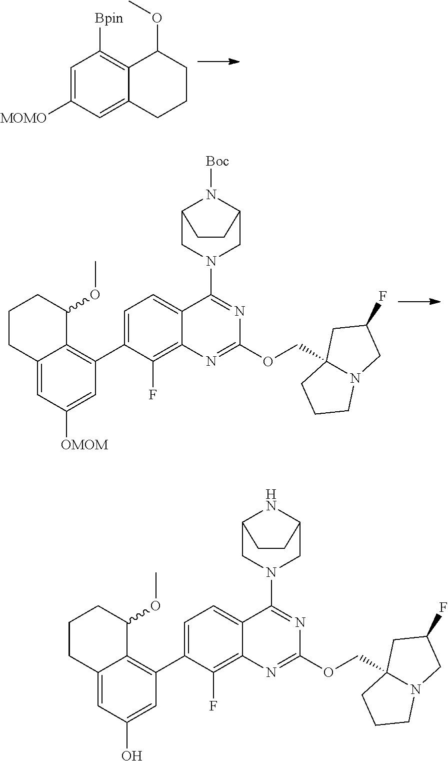

### Step 1: Synthesis of tert-butyl (1R,5S)-3-(8-fluoro-2-(((2R,7aS)-2-fluorotetrahydro-1H-pyrrolizin-7a(5H)-yl)methoxy)-7-(8-methoxy-3-(methoxymethoxy)-5,6,7,8-tetrahydronaphthalen-1-yl)quinazolin-4-yl)-3,8-diazabicyclo[3.2.1]octane-8-carboxylate

A solution of 2-(8-methoxy-3-(methoxymethoxy)-5,6,7,8-tetrahydronaphthalen-1-yl)-4,4,5,5-tetramethyl-1,3,2-dioxaborolane (20 mg, 0.057 mmol), tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (34 mg, 0.057 mmol) in 1,4-dioxane (0.8 mL) and H₂O (0.2 mL), was added cataCXium A Pd G4 (8 mg, 0.01 mmol) and K₃PO₄(36 mg, 0.16 mmol). The mixture was stirred under N₂ at 70° C. overnight. After the reaction was complete, the mixture was concentrated and purified by prep-TLC to give a white solid (29 mg, yield: 69%). MS m/z: 736.69 [M+H]⁺.

### Step 2: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-methoxy-5,6,7,8-tetrahydronaphthalen-2-ol

Compound tert-butyl (1R,5S)-3-(8-fluoro-2-(((2R,7aS)-2-fluorotetrahydro-1H-pyrrolizin-7a(5H)-yl)methoxy)-7-(8-methoxy-3-(methoxymethoxy)-5,6,7,8-tetrahydronaphthalen-1-yl)quinazolin-4-yl)-3,8-diazabicyclo[3.2.1]octane-8-carboxylate (29 mg, 0.039 mmol) was stirred in DCM (0.5 mL) and TFA (0.5 mL) under N₂ for 1h. The mixture was concentrated, extracted with ethyl acetate and saturated sodium bicarbonate. The organic phase was washed with sat. NaCl, dried with anhydrous sodium sulfate, concentrated and purified by prep-TLC to obtain a yellow solid. (1 mg, yield: 4.8%). MS m/z: 592.4 [M+H]⁺.

### Example 281: Synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-7b-methyl-1a, 2,3,7b-tetrahydro-1H-cyclopropa[a]naphthalen-5-ol

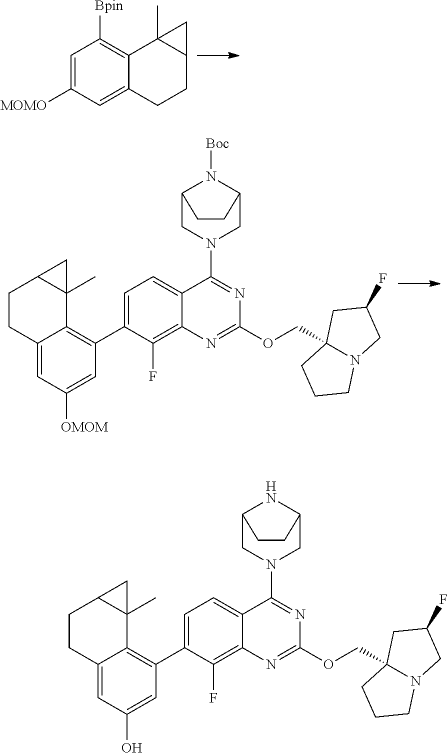

### Step 1: Synthesis of tert-butyl (1R,5S)-3-(8-fluoro-2-(((2R,7aS)-2-fluorotetrahydro-1H-pyrrolizin-7a(5H)-yl)methoxy)-7-(5-(methoxymethoxy)-7b-methyl-1a, 2,3,7b-tetrahydro-1H-cyclopropa[a]naphthalen-7-Vi)quinazolin-4-yl)-3,8-diazabicyclo[3.2.1]octane-8-carboxylate

A solution of 2-(5-(methoxymethoxy)-7b-methyl-1a, 2,3,7b-tetrahydro-1H-cyclopropa[a]naphthalen-7-yl)-4,4,5,5-tetramethyl-1,3,2-dioxaborolane (4 mg, 0.0116 mmol), tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (7 mg, 0.0116 mmol) in 1,4-dioxane (0.8 mL) and H₂O (0.2 mL), was added cataCXium A Pd G4 (2 mg, 0.002 mmol) and K₃PO₄(7 mg, 0.033 mmol). The mixture was stirred under N₂ at 70° C. overnight. After the reaction was complete, the mixture was concentrated and purified by prep-TLC to give a white solid (4.2 mg, yield: 50%).

### Step 2: Synthesis of 7-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-7b-methyl-1a, 2,3,7b-tetrahydro-1H-cyclopropane[a]naphthalen-5-ol

Compound tert-butyl (1R,5S)-3-(8-fluoro-2-(((2R,7aS)-2-fluorotetrahydro-1H-pyrrolizin-7a(5H)-yl)methoxy)-7-(5-(methoxymethoxy)-7b-methyl-1a, 2,3,7b-tetrahydro-1H-cyclopropa[a]naphthalen-7-yl)quinazolin-4-yl)-3,8-diazabicyclo[3.2.1]octane-8-carboxylate (4.2 mg, 0.005 mmol) was stirred in CH₃CN (0.5 mL) and HCl/dioxane (1 mL, 4M) under N₂ for 8h. The mixture was concentrated, extracted with ethyl acetate and saturated sodium bicarbonate. The organic phase was washed with sat. NaCl, dried with anhydrous sodium sulfate, concentrated and purified by prep-TLC to obtain a yellow solid. (1 mg, yield: 35%). MS m/z: 588.3 [M+H]⁺.

### Example 282: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-methyl-5,6,7,8-tetrahydronaphthalen-2-ol

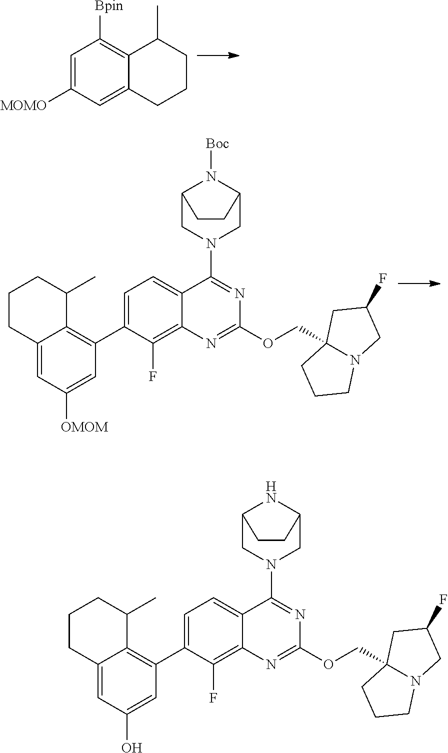

### Step 1: Synthesis of tert-butyl (1R,5S)-3-(8-fluoro-2-(((2R,7aS)-2-fluorotetrahydro-1H-pyrrolizin-7a(5H)-yl)methoxy)-7-(3-(methoxymethoxy)-8-methyl-5,6,7,8-tetrahydronaphthalen-1-yl)quinazolin-4-yl)-3,8-diazabicyclo[3.2.1]octane-8-carboxylate

A solution of 2-(3-(methoxymethoxy)-8-methyl-5,6,7,8-tetrahydronaphthalen-1-yl)-4,4,5,5-tetramethyl-1,3,2-dioxaborolane (40 mg, 0.125 mmol), tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (75 mg, 0.125 mmol) in 1,4-dioxane (2 mL) and H₂O (0.5 mL), was added cataCXium ANP G4 (18 mg, 0.025 mmol) and K₃PO₄ (80 mg, 0.375 mmol). The mixture was stirred under N₂ at 70° C. overnight. After the reaction was complete, the mixture was concentrated and purified by prep-TLC to give a white solid (69 mg, yield: 76%). MS m/z: 720.65 [M+H]⁺.

### Step 2: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-methyl-5,6,7,8-tetrahydronaphthalen-2-ol

Compound tert-butyl (1R,5S)-3-(8-fluoro-2-(((2R,7aS)-2-fluorotetrahydro-CH-pyrrolizin-7a(5H)-yl)methoxy)-7-(3-(methoxymethoxy)-8-methyl-5,6,7,8-tetrahydronaphthalen-1-yl)quinazolin-4-yl)-3,8-diazabicyclo[3.2.1]octane-8-carboxylate (30 mg, 0.04 mmol) was stirred in CH₃CN (1 mL) and HCl/dioxane (1 mL, 4M) under N₂ for 1h. The mixture was concentrated, extracted with ethyl acetate and saturated sodium bicarbonate. The organic phase was washed with sat. NaCl, dried with anhydrous sodium sulfate, concentrated and purified by prep-TLC to obtain a yellow solid. (18 mg, yield: 76%). MS m/z: 576.7 [M+H]⁺.

Examples 271-292 were obtained by using the method of Example 270.

| Ex. | Compound name | Structural formula | m/z: ES⁺ [M + H]⁺ |
|---|---|---|---|
| 283 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-1- ethyl-2,3-dihydro-1H-inden-5-ol | 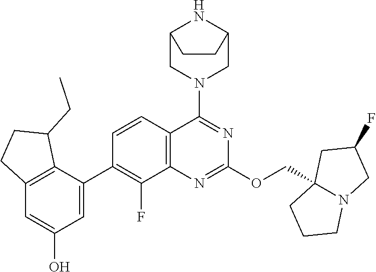 [SMILES: CCC1CCc2cc(O)cc(-c3ccc4c(N5CC6CCC(C5)N6)nc(OC[C@@]56CCCN5C[C@H](F)C6)nc4c3F)c21] | 576.5 |
| 284 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-1- ethyl-2,3-dihydro-1H-inden-5-ol, epimer-1 | 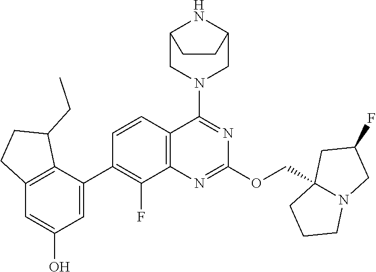 [SMILES: CCC1CCc2cc(O)cc(-c3ccc4c(N5CC6CCC(C5)N6)nc(OC[C@@]56CCCN5C[C@H](F)C6)nc4c3F)c21] | 576.5 Column: Chiralcel OJ- 3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| 285 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-1- ethyl-2,3-dihydro-1H-inden-5-ol, epimer-2 | 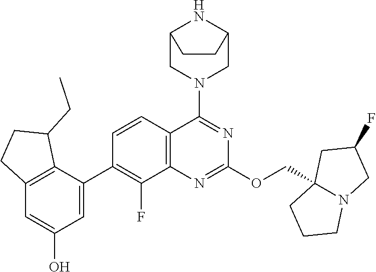 [SMILES: CCC1CCc2cc(O)cc(-c3ccc4c(N5CC6CCC(C5)N6)nc(OC[C@@]56CCCN5C[C@H](F)C6)nc4c3F)c21] | 576.5 Column: Chiralcel OJ- 3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| 286 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-1- methyl-2,3-dihydro-1H-inden-5-ol | 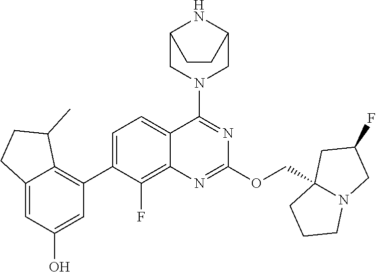 [SMILES: CC1CCc2cc(O)cc(-c3ccc4c(N5CC6CCC(C5)N6)nc(OC[C@@]56CCCN5C[C@H](F)C6)nc4c3F)c21] | 562.3 |
| 287 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- methyl-5,6,7,8-tetrahydronaphthalen-2-ol, epimer- 1 | 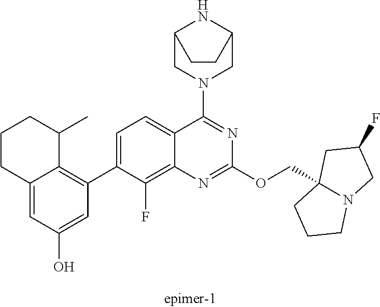 [SMILES: CC1CCCc2cc(O)cc(-c3ccc4c(N5CC6CCC(C5)N6)nc(OC[C@@]56CCCN5C[C@H](F)C6)nc4c3F)c21] | 576.7 |
| 288 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- methyl-5,6,7,8-tetrahydronaphthalen-2-ol, epimer-2 | 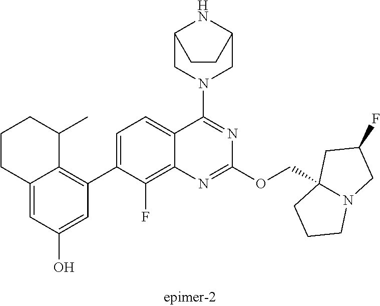 [SMILES: CC1CCCc2cc(O)cc(-c3ccc4c(N5CC6CCC(C5)N6)nc(OC[C@@]56CCCN5C[C@H](F)C6)nc4c3F)c21] | 576.7 |
| 289 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- ethyl-5,6,7,8-tetrahydronaphthalen-2-ol | 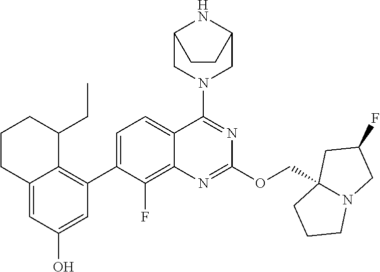 [SMILES: CCC1CCCc2cc(O)cc(-c3ccc4c(N5CC6CCC(C5)N6)nc(OC[C@@]56CCCN5C[C@H](F)C6)nc4c3F)c21] | 590.3 |
| 290 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- ethyl-5,6,7,8-tetrahydronaphthalen-2-ol, epimer-1 | 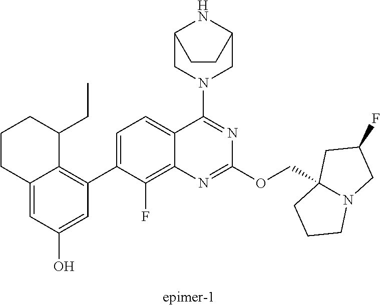 [SMILES: CCC1CCCc2cc(O)cc(-c3ccc4c(N5CC6CCC(C5)N6)nc(OC[C@@]56CCCN5C[C@H](F)C6)nc4c3F)c21] | 590.3 Column: Chiralcel OJ- 3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| 291 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- ethyl-5,6,7,8-tetrahydronaphthalen-2-ol, epimer-2 | 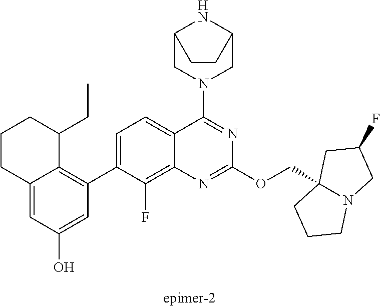 [SMILES: CCC1CCCc2cc(O)cc(-c3ccc4c(N5CC6CCC(C5)N6)nc(OC[C@@]56CCCN5C[C@H](F)C6)nc4c3F)c21] | 590.3 Column: Chiralcel OJ- 3, 150 × 4.6 mm I.D., 3 um Mobile phase: A: CO2/B: IPA (0.1% IPAm, v/v Flow rate 2.5 mL/min Column temp.: 35° C. ABPR: 2000 psi |
| 292 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-3- ethyl-2,3-dihydrobenzofuran-6-ol | 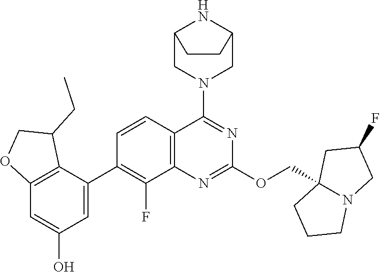 [SMILES: CCC1COc2cc(O)cc(-c3ccc4c(N5CC6CCC(C5)N6)nc(OC[C@@]56CCCN5C[C@H](F)C6)nc4c3F)c21] | 578.4 |
| 293 | 4-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-3- ethylbenzofuran-6-ol | 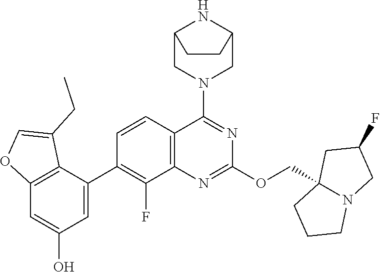 [SMILES: CCc1coc2cc(O)cc(-c3ccc4c(N5CC6CCC(C5)N6)nc(OC[C@@]56CCCN5C[C@H](F)C6)nc4c3F)c12] | 576.4 |
| 294 | 5-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)- 1,1a,6,6a-tetrahydrocyclopropa[a]inden-3-ol | 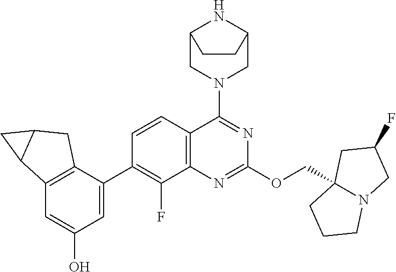 [SMILES: Oc1cc(-c2ccc3c(N4CC5CCC(C4)N5)nc(OC[C@@]45CCCN4C[C@H](F)C5)nc3c2F)c2c(c1)C1CC1C2] | 560.4 |
| 295 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)- 1a,2,3,7b-tetrahydro-1H-cyclopropa[a]naphthalen- 5-ol | 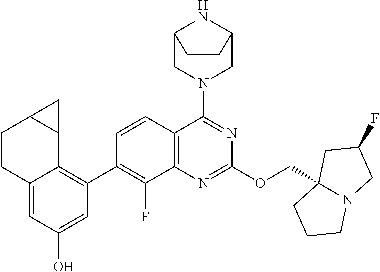 [SMILES: Oc1cc2c(c(-c3ccc4c(N5CC6CCC(C5)N6)nc(OC[C@@]56CCCN5C[C@H](F)C6)nc4c3F)c1)C1CC1CC2] | 574.4 |
| 296 | 8-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-6- hydroxy-3,4-dihydronaphthalen-1(2H)-one | 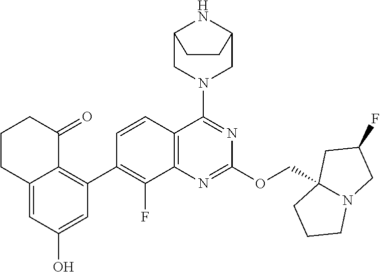 [SMILES: O=C1CCCc2cc(O)cc(-c3ccc4c(N5CC6CCC(C5)N6)nc(OC[C@@]56CCCN5C[C@H](F)C6)nc4c3F)c21] | 576.4 |
| 297 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-5- hydroxy-2,3-dihydro-1H-inden-1-one | 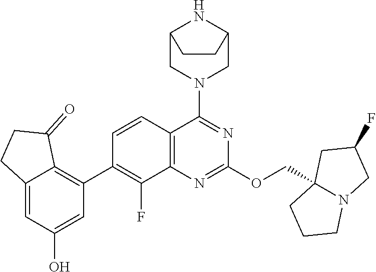 [SMILES: O=C1CCc2cc(O)cc(-c3ccc4c(N5CC6CCC(C5)N6)nc(OC[C@@]56CCCN5C[C@H](F)C6)nc4c3F)c21] | 561.4 |
| 298 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)quinazolin-7-yl)-1- methoxy-2,3-dihydro-1H-inden-5-ol | 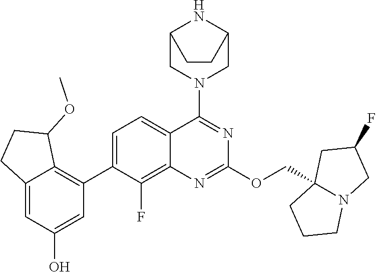 [SMILES: COC1CCc2cc(O)cc(-c3ccc4c(N5CC6CCC(C5)N6)nc(OC[C@@]56CCCN5C[C@H](F)C6)nc4c3F)c21] | 578.4 |
| 299 | 7-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)pyrido[4,3- d]pyrimidin-7-yl)-1,1-dimethyl-2,3-dihydro-1H- inden-5-ol | 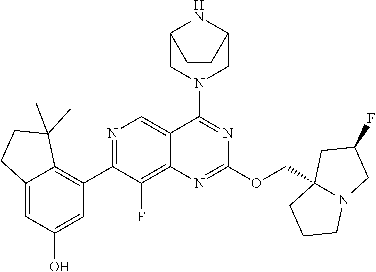 [SMILES: CC1(C)CCc2cc(O)cc(-c3ncc4c(N5CC6CCC(C5)N6)nc(OC[C@@]56CCCN5C[C@H](F)C6)nc4c3F)c21] | 577.4 |
| 300 | 4′-(4-((1R,5S)-3,8-diazabicyclo[3.2.1]octan-3-yl)- 8-fluoro-2-(((2R,7aS)-2-fluorotetrahydro-1H- pyrrolizin-7a(5H)-yl)methoxy)pyrido[4,3- d]pyrimidin-7-yl)-1′,3′-dihydrospiro[cyclopropane- 1,2′-inden]-6′-ol | 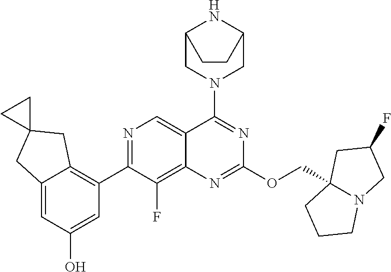 [SMILES: Oc1cc2c(c(-c3ncc4c(N5CC6CCC(C5)N6)nc(OC[C@@]56CCCN5C[C@H](F)C6)nc4c3F)c1)CC1(CC1)C2] | 575.4 |
| 301 | 4-(4-((1R,5S)-3,8-diazabicyclo[3.2.1]octan-3-yl)- 6-chloro-8-fluoro-2-(((2R,7aS)-2-fluorotetrahydro- 1H-pyrrolizin-7a(5H)-yl)methoxy)quinazolin-7- yl)-5-methyl-5,6,7,8-tetrahydronaphthalen-2-ol | 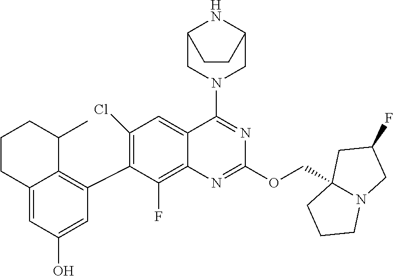 [SMILES: CC1CCCc2cc(O)cc(-c3c(Cl)cc4c(N5CC6CCC(C5)N6)nc(OC[C@@]56CCCN5C[C@H](F)C6)nc4c3F)c21] | 610.3 |
| 302 | 7-(4-((1R,5S)-3,8-diazabicyclo[3.2.1]octan-3-yl)- 6-chloro-8-fluoro-2-(((2R,7aS)-2-fluorotetrahydro- 1H-pyrrolizin-7a(5H)-yl)methoxy)quinazolin-7- yl)-1-ethyl-2,3-dihydro-1H-inden-5-ol | 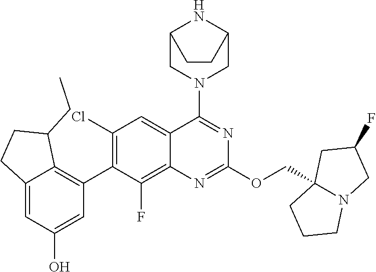 [SMILES: CCC1CCc2cc(O)cc(-c3c(Cl)cc4c(N5CC6CCC(C5)N6)nc(OC[C@@]56CCCN5C[C@H](F)C6)nc4c3F)c21] | 610.3 |
| 303 | 4-(4-((1R,5S)-3,8-diazabicyclo[3.2.1]octan-3-yl)- 6-chloro-8-fluoro-2-(((2R,7aS)-2-fluorotetrahydro- 1H-pyrrolizin-7a(5H)-yl)methoxy)quinazolin-7- yl)-5-ethyl-5,6,7,8-tetrahydronaphthalen-2-ol | 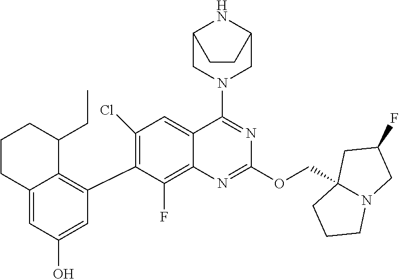 [SMILES: CCC1CCCc2cc(O)cc(-c3c(Cl)cc4c(N5CC6CCC(C5)N6)nc(OC[C@@]56CCCN5C[C@H](F)C6)nc4c3F)c21] | 624.3 |
| 304 | 7-(4-((1R,5S)-3,8-diazabicyclo[3.2.1]octan-3-yl)- 6-chloro-8-fluoro-2-(((2R,7aS)-2-fluorotetrahydro- 1H-pyrrolizin-7a(5H)-yl)methoxy)quinazolin-7- yl)-1-methyl-2,3-dihydro-1H-inden-5-ol | 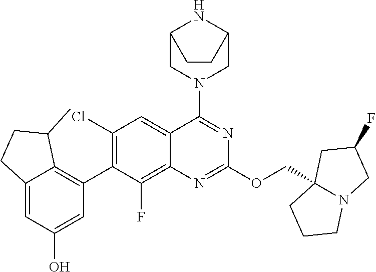 [SMILES: CC1CCc2cc(O)cc(-c3c(Cl)cc4c(N5CC6CCC(C5)N6)nc(OC[C@@]56CCCN5C[C@H](F)C6)nc4c3F)c21] | 596.3 |

### Example 305: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-7,8-dihydronaphthalen-2-ol

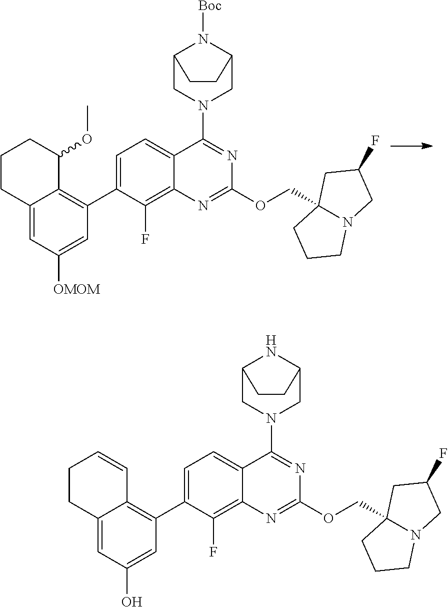

Compound tert-butyl (1R,5S)-3-(8-fluoro-2-(((2R,7aS)-2-fluorotetrahydro-1H-pyrrolizin-7a(5H)-yl)methoxy)-7-(8-methoxy-3-(methoxymethoxy)-5,6,7,8-tetrahydronaphthalen-1-yl)quinazolin-4-yl)-3,8-diazabicyclo[3.2.1]octane-8-carboxylate (17 mg, 0.023 mmol) was stirred in CH₃CN (1 mL) and HCl/dioxane (1 mL, 4M) under N₂ for 1h. The mixture was concentrated, extracted with ethyl acetate and saturated sodium bicarbonate. The organic phase was washed with sat. NaCl, dried with anhydrous sodium sulfate, concentrated and purified by prep-TLC to obtain a yellow solid. (11 mg, yield: 87%). MS m/z: 560.9 [M+H]⁺.

Example 293 was obtained by using the method of Example 294.

| Ex. | Compound name | Structural formula | m/z: ES⁺[M + H]⁺ |
|---|---|---|---|
| 306 | 4-(4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)quinazolin-7-yl)-1H-inden-6-ol | 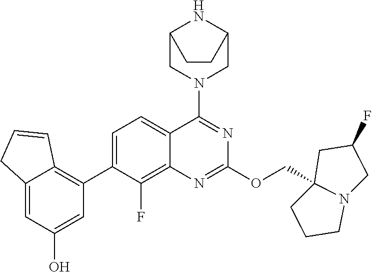 [SMILES: Oc1cc2c(c(-c3ccc4c(N5CC6CCC(C5)N6)nc(OC[C@@]56CCCN5C[C@H](F)C6)nc4c3F)c1)C=CC2] | 546.3 |

### Example 307: Synthesis of acetoxymethyl (1R,5S)-3-(7-(3-ethyl-6-hydroxy-2,3-dihydro-1H-inden-4-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

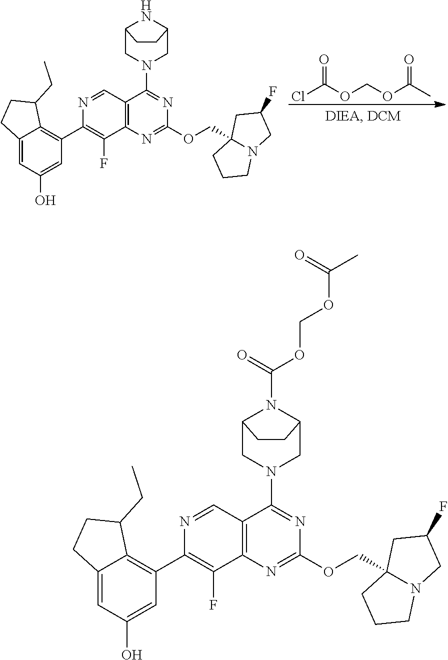

Compound 7-(4-((R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethyl-2,3-dihydro-1H-inden-5-ol (29 mg, 0.05 mmol) and DIEA (0.025 mL, 0.15 mmol) in DCM (1.5 mL) at −40° C., was added ((chlorocarbonyl)oxy)methyl acetate (9.2 mg, 0.06 mmol) in DCM (0.5 mL) dropwise. The mixture was stirred at −40° C. for 15 m². Then, warm to room temperature and diluted with DCM, concentrated, purified by prep-TLC (MeOH:DCM=1:10) to give a yellow solid (4.5 mg, 0.0065 mmol, 13% yield). MS m/z: 693.4 [M+H]⁺. ¹H NMR (400 MHz, DMSO) δ 9.31 (s, 1H), 9.07 (s, 1H), 7.24 (d, J=8.8 Hz, 1H), 6.87 (d, J=8.9 Hz, 1H), 6.72 (d, J=2.3 Hz, 1H), 6.67 (d, J=1.8 Hz, 1H), 5.84 (s, 2H), 5.33 (d, J=9.0 Hz, 1H), 5.21 (s, 1H), 4.49 (m, 2H), 4.27 (d, J=12.1 Hz, 1H), 4.11 (d, J=10.4 Hz, 1H), 4.02 (d, J=10.4 Hz, 1H), 3.65 (d, J=12.1 Hz, 1H), 3.50 (t, J=14.2 Hz, 1H), 3.16-3.05 (m, 1H), 3.01 (s, 1H), 2.94-2.72 (m, 3H), 2.24-1.92 (m, 7H), 1.93-1.71 (m, 3H), 1.63 (s, 3H), 1.24 (s, 2H), 1.11-0.92 (m, 3H), 0.53 (t, J=7.3 Hz, 3H).

Examples 296-314 were obtained by using the method of Example 295.

| | | | m/z: ES⁺ |
|---|---|---|---|
| Ex. | Compound name | Structural formula | [M + H]⁺ |
| 308 | (decanoyloxy)methyl (1R,5S)-3-(7-(8-ethynyl-7- fluoro-3-hydroxynaphthalen-1-yl)-8-fluoro-2- (((2R,7aS)-2-fluorotetrahydro-1H-pyrrolizin-7a(5H)- yl)methoxy)quinazolin-4-yl)-3,8- diazabicyclo[3.2.1]octane-8-carboxylate | 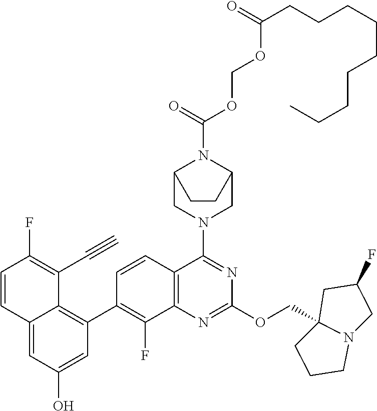 [SMILES: C#Cc1c(F)ccc2cc(O)cc(-c3ccc4c(N5CC6CCC(C5)N6C(=O)OCOC(=O)CCCCCCCCC)nc(OC[C@@]56CCCN5C[C@H](F)C6)nc4c3F)c12] | 828.5 |
| 309 | (palmitoyloxy)methyl (1R,5S)-3-(7-(8-ethynyl-7- fluoro-3-hydroxynaphthalen-1-yl)-8-fluoro-2- (((2R,7aS)-2-fluorotetrahydro-1H-pyrrolizin-7a(5H)- yl)methoxy)quinazolin-4-yl)-3,8- diazabicyclo[3.2.1]octane-8-carboxylate | 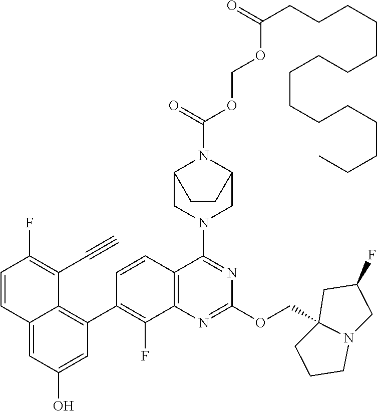 [SMILES: C#Cc1c(F)ccc2cc(O)cc(-c3ccc4c(N5CC6CCC(C5)N6C(=O)OCOC(=O)CCCCCCCCCCCCCCC)nc(OC[C@@]56CCCN5C[C@H](F)C6)nc4c3F)c12] | 911.6 |
| 310 | acetoxymethyl (1R,5S)-3-(7-(8-ethynyl-7-fluoro-3- hydroxynaphthalen-1-yl)-8-fluoro-2-(((2R,7aS)-2- fluorotetrahydro-1H-pyrrolizin-7a(5H)- yl)methoxy)quinazolin-4-yl)-3,8- diazabicyclo[3.2.1]octane-8-carboxylate | 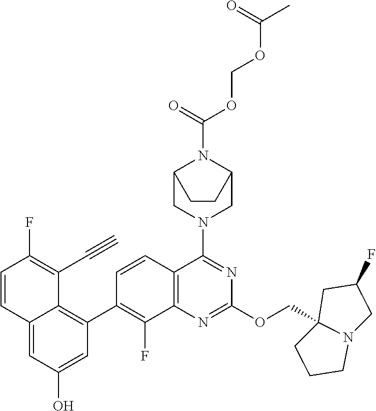 [SMILES: C#Cc1c(F)ccc2cc(O)cc(-c3ccc4c(N5CC6CCC(C5)N6C(=O)OCOC(C)=O)nc(OC[C@@]56CCCN5C[C@H](F)C6)nc4c3F)c12] | 716.4 |
| 311 | (decanoyloxy)methyl (1R,5S)-3-(7-(3-ethyl-6-hydroxy- 2,3-dihydro-1H-inden-4-yl)-8-fluoro-2-(((2R,7aS)-2- fluorotetrahydro-1H-pyrrolizin-7a(5H)- yl)methoxy)pyrido[4,3-d]pyrimidin-4-yl)-3,8- diazabicyclo[3.2.1]octane-8-carboxylate | 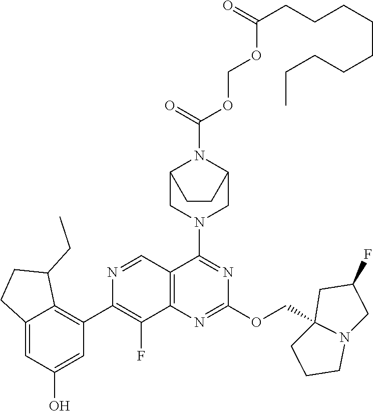 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(CC)CC5)ncc13)C2] | 805.6 |
| 312 | (decanoyloxy)methyl (1R,5S)-3-(7-(3-ethyl-6-hydroxy- 2,3-dihydro-1H-inden-4-yl)-8-fluoro-2-(((2R,7aS)-2- fluorotetrahydro-1H-pyrrolizin-7a(5H)- yl)methoxy)quinazolin-4-yl)-3,8- diazabicyclo[3.2.1]octane-8-carboxylate | 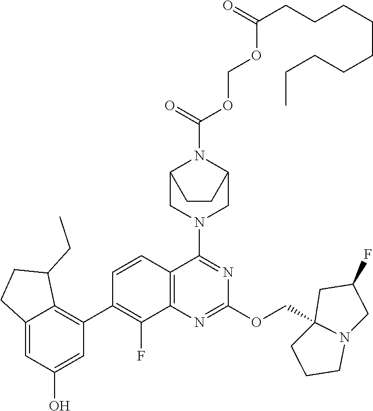 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(CC)CC5)ccc13)C2] | 804.6 |
| 313 | (palmitoyloxy)methyl (1R,5S)-3-(7-(3-ethyl-6- hydroxy-2,3-dihydro-1H-inden-4-yl)-8-fluoro-2- (((2R,7aS)-2-fluorotetrahydro-1H-pyrrolizin-7a(5H)- yl)methoxy)pyrido[4,3-d]pyrimidin-4-yl)-3,8- diazabicyclo[3.2.1]octane-8-carboxylate | 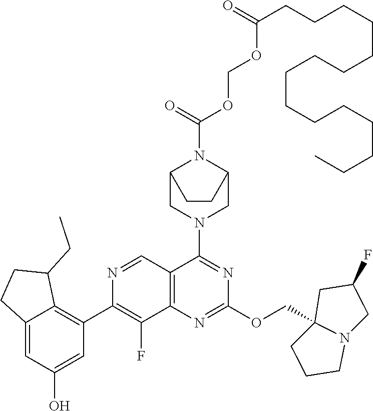 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(CC)CC5)ncc13)C2] | 889.7 |
| 314 | (palmitoyloxy)methyl (1R,5S)-3-(7-(3-ethyl-6- hydroxy-2,3-dihydro-1H-inden-4-yl)-8-fluoro-2- (((2R,7aS)-2-fluorotetrahydro-1H-pyrrolizin-7a(5H)- yl)methoxy)quinazolin-4-yl)-3,8- diazabicyclo[3.2.1]octane-8-carboxylate | 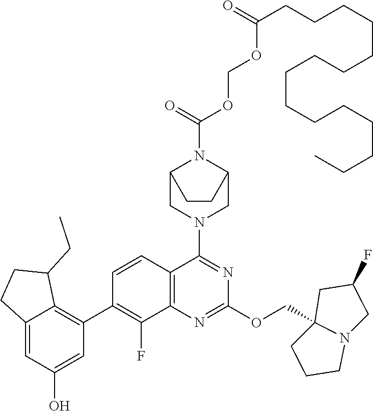 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(CC)CC5)ccc13)C2] | 888.7 |
| 315 | acetoxymethyl (1R,5S)-3-(8-fluoro-2-(((2R,7aS)-2- fluorotetrahydro-1H-pyrrolizin-7a(5H)-yl)methoxy)-7- (3-hydroxy-8-methyl-5,6,7,8-tetrahydronaphthalen-1- yl)pyrido[4,3-d]pyrimidin-4-yl)-3,8- diazabicyclo[3.2.1]octane-8-carboxylate | 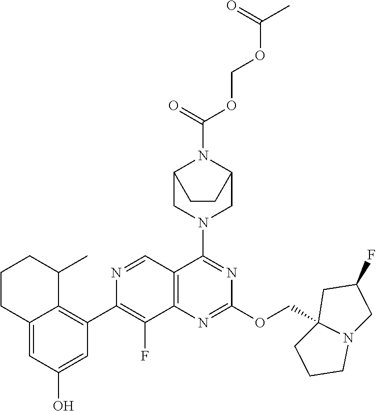 [SMILES: CC(=O)OCOC(=O)N1C2CCC1CN(c1nc(OC[C@@]34CCCN3C[C@H](F)C4)nc3c(F)c(-c4cc(O)cc5c4C(C)CCC5)ncc13)C2] | 693.3 |
| 316 | acetoxymethyl (1R,5S)-3-(8-fluoro-2-(((2R,7aS)-2- fluorotetrahydro-1H-pyrrolizin-7a(5H)-yl)methoxy)-7- (3-hydroxy-8-methyl-5,6,7,8-tetrahydronaphthalen-1- yl)quinazolin-4-yl)-3,8-diazabicyclo[3.2.1]octane-8- carboxylate | 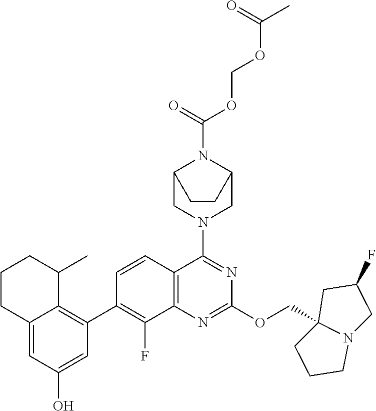 [SMILES: CC(=O)OCOC(=O)N1C2CCC1CN(c1nc(OC[C@@]34CCCN3C[C@H](F)C4)nc3c(F)c(-c4cc(O)cc5c4C(C)CCC5)ccc13)C2] | 692.8 |
| 317 | (decanoyloxy)methyl (1R,5S)-3-(8-fluoro-2- (((2R,7aS)-2-fluorotetrahydro-1H-pyrrolizin-7a(5H)- yl)methoxy)-7-(3-hydroxy-8-methyl-5,6,7,8- tetrahydronaphthalen-1-yl)pyrido[4,3-d]pyrimidin-4- yl)-3,8-diazabicyclo[3.2.1]octane-8-carboxylate | 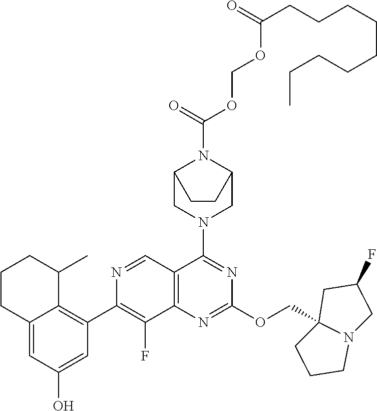 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(C)CCC5)ncc13)C2] | 805.5 |
| 318 | (decanoyloxy)methyl (1R,5S)-3-(8-fluoro-2- (((2R,7aS)-2-fluorotetrahydro-1H-pyrrolizin-7a(5H)- yl)methoxy)-7-(3-hydroxy-8-methyl-5,6,7,8- tetrahydronaphthalen-1-yl)quinazolin-4-yl)-3,8- diazabicyclo[3.2.1]octane-8-carboxylate | 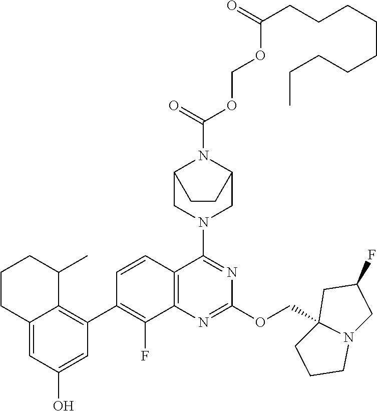 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(C)CCC5)ccc13)C2] | 804.5 |
| 319 | (palmitoyloxy)methyl (1R,5S)-3-(8-fluoro-2- (((2R,7aS)-2-fluorotetrahydro-1H-pyrrolizin-7a(5H)- yl)methoxy)-7-(3-hydroxy-8-methyl-5,6,7,8- tetrahydronaphthalen-1-yl)pyrido[4,3-d]pyrimidin-4- yl)-3,8-diazabicyclo[3.2.1]octane-8-carboxylate | 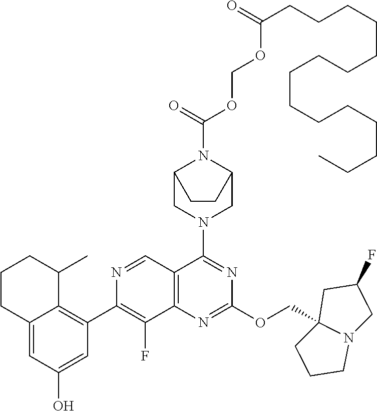 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(C)CCC5)ncc13)C2] | 889.4 |
| 320 | (palmitoyloxy)methyl (1R,5S)-3-(8-fluoro-2- (((2R,7aS)-2-fluorotetrahydro-1H-pyrrolizin-7a(5H)- yl)methoxy)-7-(3-hydroxy-8-methyl-5,6,7,8- tetrahydronaphthalen-1-yl)quinazolin-4-yl)-3,8- diazabicyclo[3.2.1]octane-8-carboxylate | 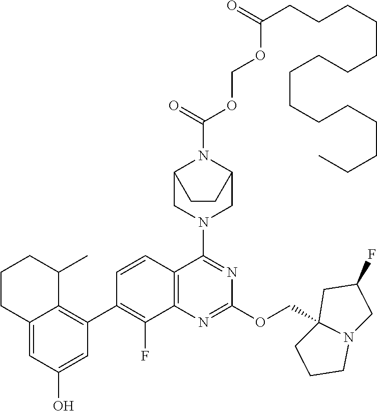 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(C)CCC5)ccc13)C2] | 888.4 |
| 321 | acetoxymethyl (1R,5S)-3-(7-(8-ethyl-3-hydroxy- 5,6,7,8-tetrahydronaphthalen-1-yl)-8-fluoro-2- (((2R,7aS)-2-fluorotetrahydro-1H-pyrrolizin-7a(5H)- yl)methoxy)pyrido[4,3-d]pyrimidin-4-yl)-3,8- diazabicyclo[3.2.1]octane-8-carboxylate | 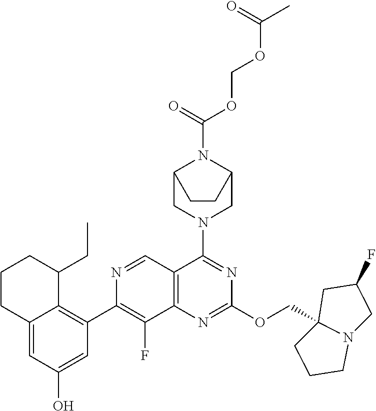 [SMILES: CCC1CCCc2cc(O)cc(-c3ncc4c(N5CC6CCC(C5)N6C(=O)OCOC(C)=O)nc(OC[C@@]56CCCN5C[C@H](F)C6)nc4c3F)c21] | 707.4 |
| 322 | (decanoyloxy)methyl (1R,5S)-3-(7-(8-ethyl-3-hydroxy- 5,6,7,8-tetrahydronaphthalen-1-yl)-8-fluoro-2- (((2R,7aS)-2-fluorotetrahydro-1H-pyrrolizin-7a(5H)- yl)methoxy)pyrido[4,3-d]pyrimidin-4-yl)-3,8- diazabicyclo[3.2.1]octane-8-carboxylate | 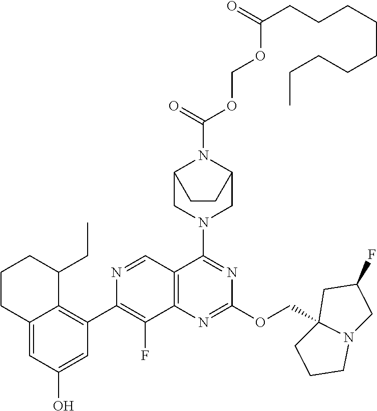 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(CC)CCC5)ncc13)C2] | 819.6 |
| 323 | (palmitoyloxy)methyl (1R,5S)-3-(7-(8-ethyl-3- hydroxy-5,6,7,8-tetrahydronaphthalen-1-yl)-8-fluoro-2- (((2R,7aS)-2-fluorotetrahydro-1H-pyrrolizin-7a(5H)- yl)methoxy)pyrido[4,3-d]pyrimidin-4-yl)-3,8- diazabicyclo[3.2.1]octane-8-carboxylate | 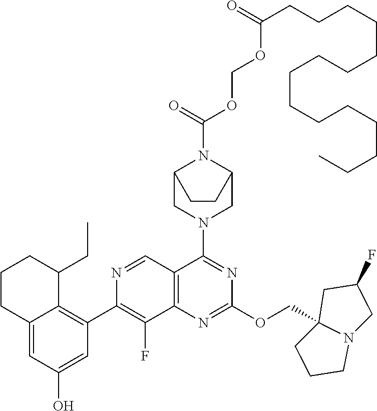 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(CC)CCC5)ncc13)C2] | 903.7 |
| 324 | acetoxymethyl (1R,5S)-3-(8-fluoro-2-(((2R,7aS)-2- fluorotetrahydro-1H-pyrrolizin-7a(5H)-yl)methoxy)-7- (6-hydroxy-3-methyl-2,3-dihydro-1H-inden-4- yl)pyrido[4,3-d]pyrimidin-4-yl)-3,8- diazabicyclo[3.2.1]octane-8-carboxylate | 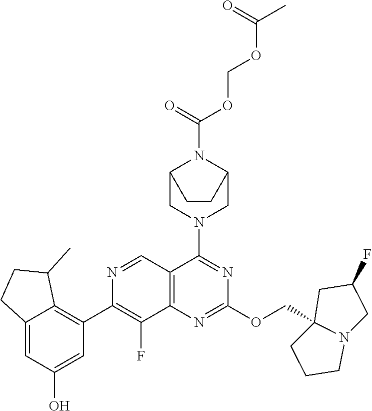 [SMILES: CC(=O)OCOC(=O)N1C2CCC1CN(c1nc(OC[C@@]34CCCN3C[C@H](F)C4)nc3c(F)c(-c4cc(O)cc5c4C(C)CC5)ncc13)C2] | 679.4 |
| 325 | (decanoyloxy)methyl (1R,5S)-3-(8-fluoro-2- (((2R,7aS)-2-fluorotetrahydro-1H-pyrrolizin-7a(5H)- yl)methoxy)-7-(6-hydroxy-3-methyl-2,3-dihydro-1H- inden-4-yl)pyrido[4,3-d]pyrimidin-4-yl)-3,8- diazabicyclo[3.2.1]octane-8-carboxylate | 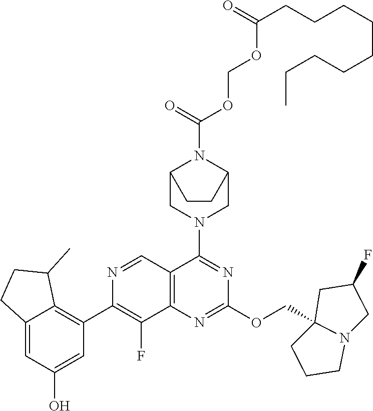 [SMILES: CCCCCCCCCC(=O)OCOC(=O)N1C2CCC1CN(c1nc(OC[C@@]34CCCN3C[C@H](F)C4)nc3c(F)c(-c4cc(O)cc5c4C(C)CC5)ncc13)C2] | 791.5 |
| 326 | (palmitoyloxy)methyl (1R,5S)-3-(8-fluoro-2- (((2R,7aS)-2-fluorotetrahydro-1H-pyrrolizin-7a(5H)- yl)methoxy)-7-(6-hydroxy-3-methyl-2,3-dihydro-1H- inden-4-yl)pyrido[4,3-d]pyrimidin-4-yl)-3,8- diazabicyclo[3.2.1]octane-8-carboxylate | 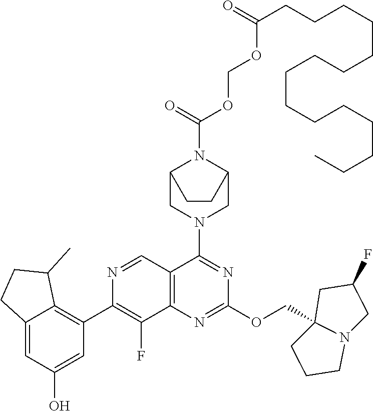 [SMILES: CCCCCCCCCCCCCCCC(=O)OCOC(=O)N1C2CCC1CN(c1nc(OC[C@@]34CCCN3C[C@H](F)C4)nc3c(F)c(-c4cc(O)cc5c4C(C)CC5)ncc13)C2] | 875.6 |

### Example 327: Synthesis of 4-(4-((1R,5S)-3,8-diazabicyclo[3.2.1]octan-3-yl)-8-cyclopropoxy-2-(((2R,7aS)-2-fluorotetrahydro-1H-pyrrolizin-7a(5H)-yl)methoxy)quinazolin-7-yl)-5-ethynyl-6-fluoronaphthalen-2-ol

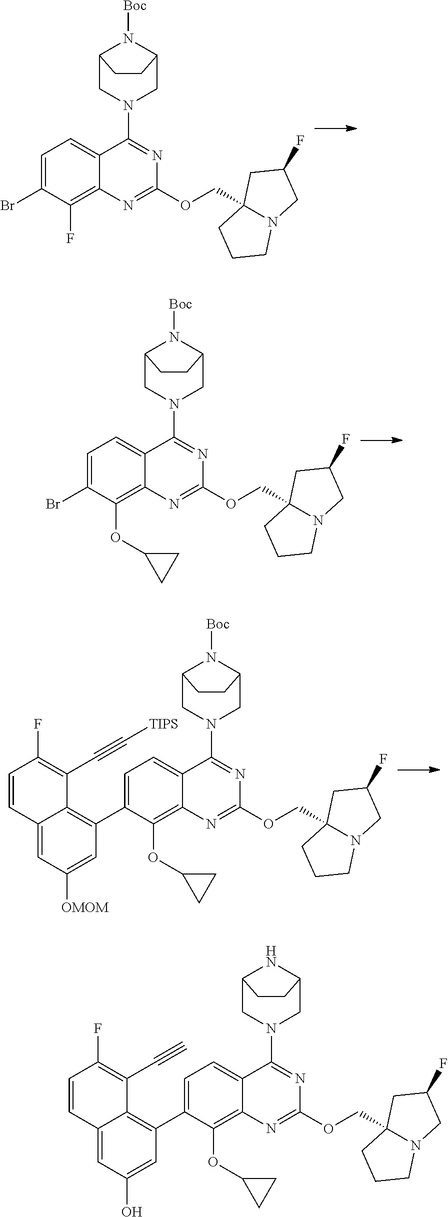

### Step 1: Synthesis of tert-butyl (1R,5S)-3-(7-bromo-8-cyclopropoxy-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

Cyclopropanol (99 mg, 1.68 mmol) was dissolved in tetrahydrofuran (5 mL) and cooled to 0° C., KHMDS (1 M/L, 1.7 ml) was added dropwise, and after 30 min, tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (200 mg, 0.337 mmol) was added; and the mixture was reacted at room temperature for 1 h, and the reaction was then quenched by adding a saturated ammonium chloride aqueous solution, extracted with ethyl acetate, concentrated and then purified by prep-TLC to obtain a white solid (210 mg, yield: 95%). MS m/z: 632.22 [M+H]⁺.

### Step 2: Synthesis of tert-butyl (1R,5S)-3-(8-cyclopropoxy-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

Compound tert-butyl (1R,5S)-3-(7-bromo-8-cyclopropoxy-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (70 mg, 0.11 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (73 mg, 0.14 mmol), Potassium carbonate (31 mg, 0.22 mmol) and Pd (dppf) Cl₂ (8 mg, 0.01 mmol) were stirred in 1,4-dioxane/water=5/1 (6 mL) under nitrogen atmosphere at 100° C. for 12 hours. After the reaction was completed, cooled to room temperature, the mixture was diluted with water, extracted with ethyl acetate. The organic phase was dried with anhydrous sodium sulfate, concentrated under reduced pressure to obtain the crude product. The crude product was purified by prep-TLC to obtain a white solid. (33 mg, yield: 36%). MS m/z: 938.5 [M+H]⁺.

### Step 3: Synthesis of 4-(4-((1R,5S)-3,8-diazabicyclo[3.2.1]octan-3-yl)-8-cyclopropoxy-2-(((2R,7aS)-2-fluorotetrahydro-1H-pyrrolizin-7a(5H)-yl)methoxy)quinazolin-7-yl)-5-ethynyl-6-fluoronaphthalen-2-ol

Compound tert-butyl (1R,5S)-3-(8-cyclopropoxy-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (30 mg, 0.031 mmol) was stirred in HCl/dioxane (5 mL, 4M) for 1h. The mixture was concentrated, extracted with DCM and saturated sodium bicarbonate. The organic phase was dried with anhydrous sodium sulfate, concentrated and purified by prep-TLC to obtain a yellow solid. (15 mg, yield: 80%). MS m/z: 638.3 [M+H]⁺.

Examples 316-322 were obtained by using the method of Example 315.

| | | | m/z: ES⁺ |
|---|---|---|---|
| Ex. | Compound name | Structural formula | [M + H]⁺ |
| 328 | 4-((1R,5S)-3,8-diazabicyclo[3.2.1]octan-3-yl)-7-(8- ethynyl-7-fluoro-3-hydroxynaphthalen-1-yl)-2- (((2R,7aS)-2-fluorotetrahydro-1H-pyrrolizin-7a(5H)- yl)methoxy)quinazolin-8-ol | 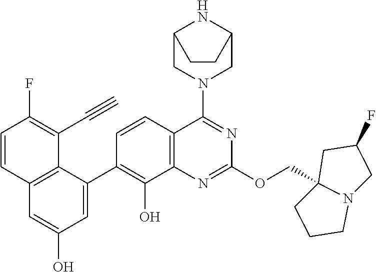 [SMILES: C#Cc1c(F)ccc2cc(O)cc(-c3ccc4c(N5CC6CCC(C5)N6)nc(OC[C@@]56CCCN5C[C@H](F)C6)nc4c3O)c12] | 597.4 |
| 329 | 4-(4-((1R,5S)-3,8-diazabicyclo[3.2.1]octan-3-yl)-6- chloro-8-cyclopropoxy-2-(((2R,7aS)-2- fluorotetrahydro-1H-pyrrolizin-7a(5H)- yl)methoxy)quinazolin-7-yl)naphthalen-2-ol | 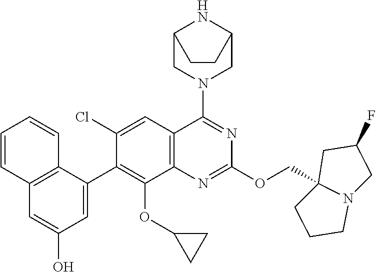 [SMILES: Oc1cc(-c2c(Cl)cc3c(N4CC5CCC(C4)N5)nc(OC[C@@]45CCCN4C[C@H](F)C5)nc3c2OC2CC2)c2ccccc2c1] | 630.4 |
| 330 | 4-(4-((1R,5S)-3,8-diazabicyclo[3.2.1]octan-3-yl)-6- chloro-8-cyclopropoxy-2-(((2R,7aS)-2- fluorotetrahydro-1H-pyrrolizin-7a(5H)- yl)methoxy)quinazolin-7-yl)-5-ethynyl-6- fluoronaphthalen-2-ol | 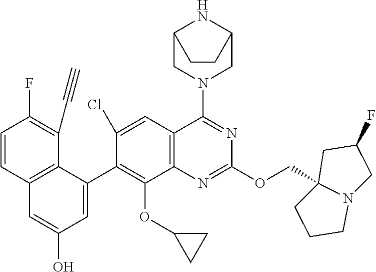 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3OC3CC3)c12] | 672.4 |
| 331 | 4-(4-((1R,5S)-3,8-diazabicyclo[3.2.1]octan-3-yl)-6- chloro-2-(((2R,7aS)-2-fluorotetrahydro-1H-pyrrolizin- 7a(5H)-yl)methoxy)-8-(2,2,2- trifluoroethoxy)quinazolin-7-yl)-5-ethynyl-6- fluoronaphthalen-2-ol | 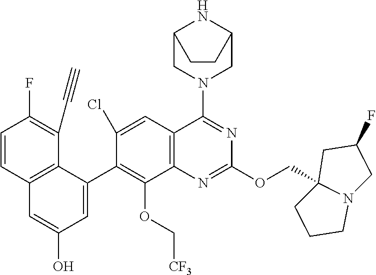 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3OCC(F)(F)F)c12] | 714.3 |
| 332 | 4-((1R,5S)-3,8-diazabicyclo[3.2.1]octan-3-yl)-7-(8- ethynyl-7-fluoro-3-hydroxynaphthalen-1-yl)-2- (((2R,7aS)-2-fluorotetrahydro-1H-pyrrolizin-7a(5H)- yl)methoxy)pyrido[4,3-d]pyrimidin-8-ol | 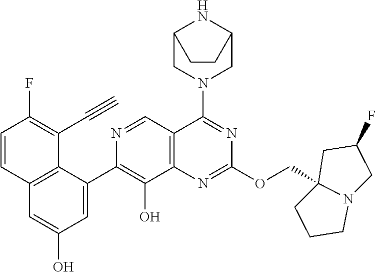 [SMILES: C#Cc1c(F)ccc2cc(O)cc(-c3ncc4c(N5CC6CCC(C5)N6)nc(OC[C@@]56CCCN5C[C@H](F)C6)nc4c3O)c12] | 599.4 |
| 333 | 4-(4-((1R,5S)-3,8-diazabicyclo[3.2.1]octan-3-yl)-2- (((2R,7aS)-2-fluorotetrahydro-1H-pyrrolizin-7a(5H)- yl)methoxy)-8-(2,2,2-trifluoroethoxy)pyrido[4,3- d]pyrimidin-7-yl)-5-ethynyl-6-fluoronaphthalen-2-ol | 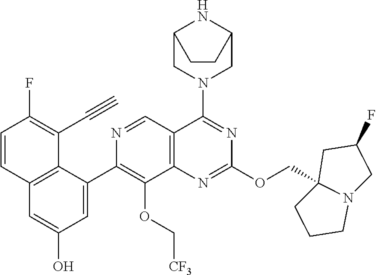 [SMILES: C#Cc1c(F)ccc2cc(O)cc(-c3ncc4c(N5CC6CCC(C5)N6)nc(OC[C@@]56CCCN5C[C@H](F)C6)nc4c3OCC(F)(F)F)c12] | 681.4 |
| 334 | 4-(4-((1R,5S)-3,8-diazabicyclo[3.2.1]octan-3-yl)-8- cyclopropoxy-2-(((2R,7aS)-2-fluorotetrahydro-1H- pyrrolizin-7a(5H)-yl)methoxy)pyrido[4,3- d]pyrimidin-7-yl)-5-ethynyl-6- fluoronaphthalen-2-ol | 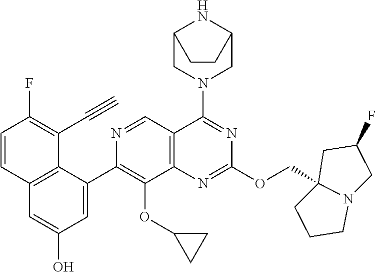 [SMILES: C#Cc1c(F)ccc2cc(O)cc(-c3ncc4c(N5CC6CCC(C5)N6)nc(OC[C@@]56CCCN5C[C@H](F)C6)nc4c3OC3CC3)c12] | 639.4 |

### Example 335: Synthesis of 4-(4-((1R,5S)-3,8-diazabicyclo[3.2.1]octan-3-yl)-3,8-difluoro-2-(((2R,7aS)-2-fluorotetrahydro-1H-pyrrolizin-7a(5H)-yl)methoxy)quinolin-7-yl)-5-ethynyl-6-fluoronanhthalen-2-ol

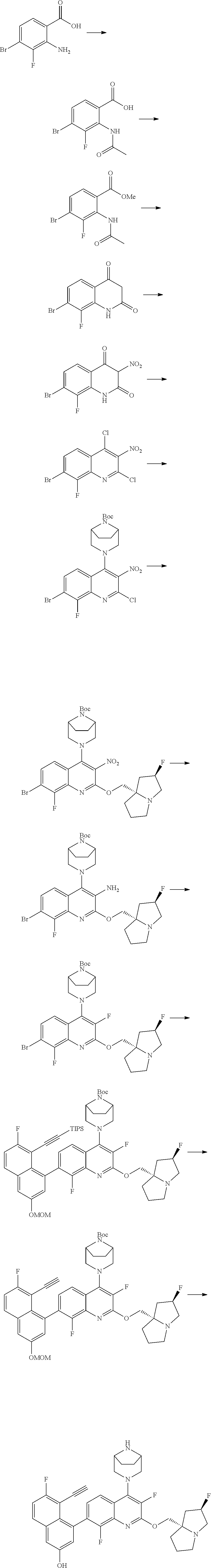

### Step 1: Synthesis of 2-acetamido-4-bromo-3-fluorobenzoic acid

Compound 2-amino-4-bromo-3-fluorobenzoic acid (1 g, 4.27 mmol) was stirred in acetic anhydride (10 mL) at 80° C. for 5h. The mixture was concentrated to obtain a white solid. (1.2 g, yield: 100%). ¹HNMR (400 MHz, CDCl₃) δ 7.86 (dd, J=8.2, 1.6 Hz, 1H), 7.68 (dd, J=8.5, 5.9 Hz, 1H), 2.54 (s, 3H).

### Step 2: Synthesis of methyl 2-acetamido-4-bromo-3-fluorobenzoate

Compound 2-acetamido-4-bromo-3-fluorobenzoic acid (200 mg, 0.8 mmol), EDCI (183 mg, 0.96 mmol) and DMAP (4.88 mg, 0.04 mmol) were stirred in DCM (10 mL) and MeOH (1 mL) at room temperature for 10 h. The mixture was extracted with DCM and H₂O, concentrated and purified by column chromatography to obtain a white solid. (200 mg, yield: 86.5%). ¹HNMR (400 MHz, CDCl₃) δ 8.98 (s, 1H), 7.62 (dd, J=8.6, 1.6 Hz, 1H), 7.44 (dd, J=8.6, 6.1 Hz, 1H), 3.92 (s, 3H), 2.25 (s, 3H).

### Step 3: Synthesis of 7-bromo-8-fluoroquinoline-2,4(1H,3H)-dione

KHMDS (12.4 mL, 12.4 mmol) in a single-mouth bottle was added methyl 2-acetamido-4-bromo-3-fluorobenzoate (1.2 g, 4.1 mmol) in THF under Ar at −78° C. The mixture was stirred at room temperature for 1 h, then the reaction liquid was poured onto crushed ice. White solid was precipitated out when pH was adjusted to 2.0 with 6N HCl. It was filtered and dried to give a white solid. (800 mg, yield: 75.4%). ¹H NMR (400 MHz, DMSO-d₆) δ 11.71 (s, 1H), 11.42 (s, 1H), 7.53 (d, J=8.7 Hz, 1H), 7.39 (t, J=7.3 Hz, 1H), 5.80 (s, 1H).

### Step 4: Synthesis of 7-bromo-8-fluoro-3-nitroquinoline-2,4(1H,3H)-dione

Compound 7-bromo-8-fluoroquinoline-2,4(1H,3H)-dione (280 mg, 1.08 mmol) in AcOH (10 mL), was added concentrated nitric acid (201 mg, 2.17 mmol). The mixture was stirred at 80° C. for 1 h, then the reaction liquid was poured onto crushed ice, extracted with EA, concentrated to give a yellow solid. (200 mg, yield: 60.9%). ¹H NMR (400 MHz, DMSO-d₆) δ 11.66 (s, 1H), 7.73 (dd, J=8.8, 1.4 Hz, 1H), 7.45 (dd, J=8.7, 6.1 Hz, 1H).

### Step 5: Synthesis of 7-bromo-2,4-dichloro-8-fluoro-3-nitroquinoline

Compound 7-bromo-8-fluoro-3-nitroquinoline-2,4(1H,3H)-dione (200 mg, 0.66 mmol) and DIPEA (170 mg, 1.32 mmol) were stirred in POCl₃ (10 mL) at 100° C. for 10 h. The mixture was concentrated, extracted with EA and H₂O, concentrated and purified by column chromatography to obtain a yellow solid. (100 mg, yield: 44.6%). ¹H NMR (400 MHz, DMSO-d₆) δ 8.22 (dd, J=9.1, 6.3 Hz, 1H), 8.11 (dd, J=9.1, 1.4 Hz, 1H).

### Step 6: Synthesis of tert-butyl (1R,5S)-3-(7-bromo-2-chloro-8-fluoro-3-nitroquinolin-4-yl)-3,8-diazabicyclo[3.2.1]octane-8-carboxylate

Compound 7-bromo-2,4-dichloro-8-fluoro-3-nitroquinoline (50 mg, 0.147 mmol) in DMF (10 mL), was added DIPEA (28.5 mg, 0.22 mmol) and tert-butyl (1R,5S)-3,8-diazabicyclo[3.2.1]octane-8-carboxylate (31.2 mg, 0.147 mmol). The mixture was stirred at room temperature for 2 h. After the reaction was complete, the reaction liquid was diluted with water and extracted with ethyl acetate, and the organic phase was washed with a saturated aqueous NaCl solution, dried with anhydrous sodium sulfate, concentrated and separated by prep-TLC to obtain a light yellow solid. (36 mg, yield: 47.5%).

### Step 7: Synthesis of tert-butyl (1R,5S)-3-(7-bromo-8-fluoro-2-(((2R,7aS)-2-fluorotetrahydro-1H-pyrrolizin-7a(5H)-yl)methoxy)-3-nitroquinolin-4-yl)-3,8-diazabicyclo[3.2.1]octane-8-carboxylate

The compound ((2R,7aS)-2-fluorotetrahydro-1H-pyrrolizin-7a(5H)-yl)methanol (14.4 mg, 0.09 mmol) was dissolved in anhydrous tetrahydrofuran (10 mL), 60% NaH (3.3 mg, 0.84 mmol) was added under ice-water bath cooling condition and reacted at 0° C. under stirring for 30 min, and tert-butyl (1R,5S)-3-(7-bromo-2-chloro-8-fluoro-3-nitroquinolin-4-yl)-3,8-diazabicyclo[3.2.1]octane-8-carboxylate (36 mg, 0.07 mmol) was added. After the reaction was complete, the reaction was quenched with cold water, extracted with ethyl acetate, and the organic phase was washed with a saturated aqueous NaCl solution, dried with anhydrous sodium sulfate, concentrated, and separated by column chromatography to obtain a yellow solid. (35 mg, yield: 78.6%). MS m/z: 638.1 [M+H]⁺.

### Step 8: Synthesis of tert-butyl (1R,5S)-3-(3-amino-7-bromo-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)-3-nitroquinolin-4-yl)-3,8-diazabicyclo[3.2.1]octane-8-carboxylate (200 mg, 0.313 mmol) in EtOH (10 mL) and H₂O (2 mL) was added iron powder (87.7 mg, 1.56 mmol) and AcOH (131.5 mg, 2.19 mmol). The mixture was stirred at room temperature for 2 h. The reaction was filtered, extracted with ethyl acetate, concentrated, and separated by column chromatography to obtain a yellow solid. (150 mg, yield: 78.9%).

### Step 9: Synthesis of tert-butyl (1R,5S)-3-(7-bromo-3,8-difluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

Compound tert-butyl (1R,5S)-3-(3-amino-7-bromo-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (150 mg, 0.247 mmol) in pyridine hydrogen fluoride (5 mL) at 0° C., was added NaNO₂ (22 mg, 0.321 mmol). The mixture was stirred at 80° C. for 10 h. After the reaction was complete, the reaction liquid was diluted with water and extracted with ethyl acetate, concentrated and separated by column chromatography to obtain a light yellow solid. (100 mg, yield: 66.7%).

### Step 10: Synthesis of tert-butyl (1R,5S)-3-(3,8-difluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

Compound tert-butyl (1R,5S)-3-(7-bromo-3,8-difluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (100 mg, 0.163 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (125.8 mg, 0.245 mmol) and Cs₂CO₃ (159 mg, 0.489 mmol) were dissolved in 1,4-dioxane and water (3 ml, 5/1), and after displacement with nitrogen, Pd(dppf)Cl₂ (11.9 mg, 0.0163 mmol) was added. The reaction system was stirred at 100° C. for 12 h. After the reaction was complete, the reaction system was extracted with ethyl acetate and separated, the organic phase was dried, and the solvent was then removed under reduced pressure to obtain a crude product. The crude product was separated by prep-TLC to obtain a white solid (80 mg, yield: 53.7%). MS m/z: 917.5 [M+H]+

### Step 11: Synthesis of 4-(4-((1R,5S)-3,8-diazabicyclo[3.2.1]octan-3-yl)-3,8-difluoro-2-(((2R,7aS)-2-fluorotetrahydro-1H-pyrrolizin-7a(5H)-yl)methoxy)quinolin-7-yl)-5-ethynyl-6-fluoronaphthalen-2-ol

Compound tert-butyl (1R,5S)-3-(3,8-difluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (80 mg, 0.087 mmol) and CsF (66 mg, 0.435 mmol) in DMF (3 mL) were stirred at room temperature for 2 h. After the reaction was complete, the reaction was diluted with water, extracted with ethyl acetate, and the organic phase was washed with a saturated aqueous NaCl solution, dried with anhydrous sodium sulfate, concentrated to give the crude product. Then the crude product was dissolved with CH₃CN (2 mL), HCl/1,4-dioxane (4M, 1 mL) was added and stirred at room temperature for 30 min. After the reaction was complete, pH of the solution was adjusted to 7 with saturated sodium bicarbonate aqueous solution, extracted with DCM, and the organic phase was dried with anhydrous sodium sulfate, concentrated, purified by prep-TLC (DCM/MeOH(7N NH₃)=10/1) to give a yellow solid (25 mg, yield: 46.6%). MS m/z: 617.2 [M+H]⁺

### Example 336: Synthesis of 4-(4-((1R,5S)-3,8-diazabicyclo[3.2.1]octan-3-yl)-8-fluoro-2-(((2R,7aS)-2-fluorotetrahvdro-1H-pyrrolizin-7a(5H)-yl)methoxy)quinazolin-7-yl)-6-fluoro-5-(methylthio)naphthalen-2-ol

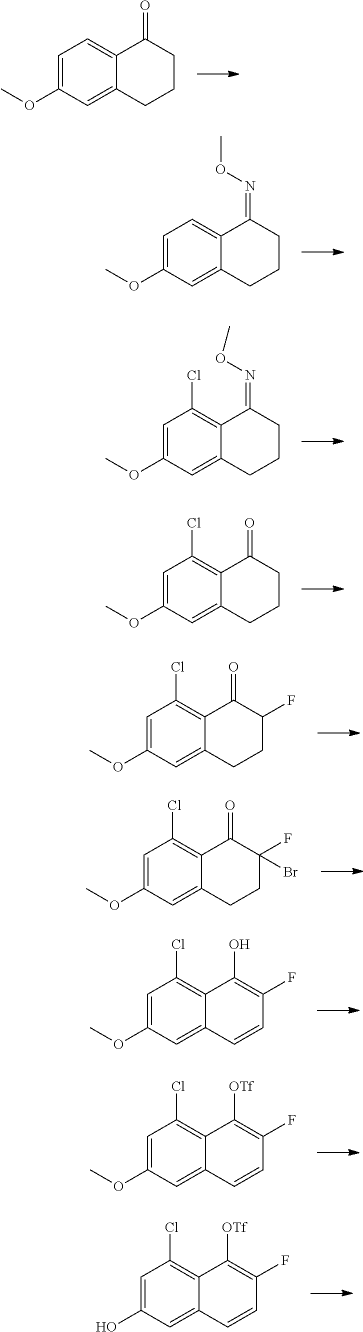

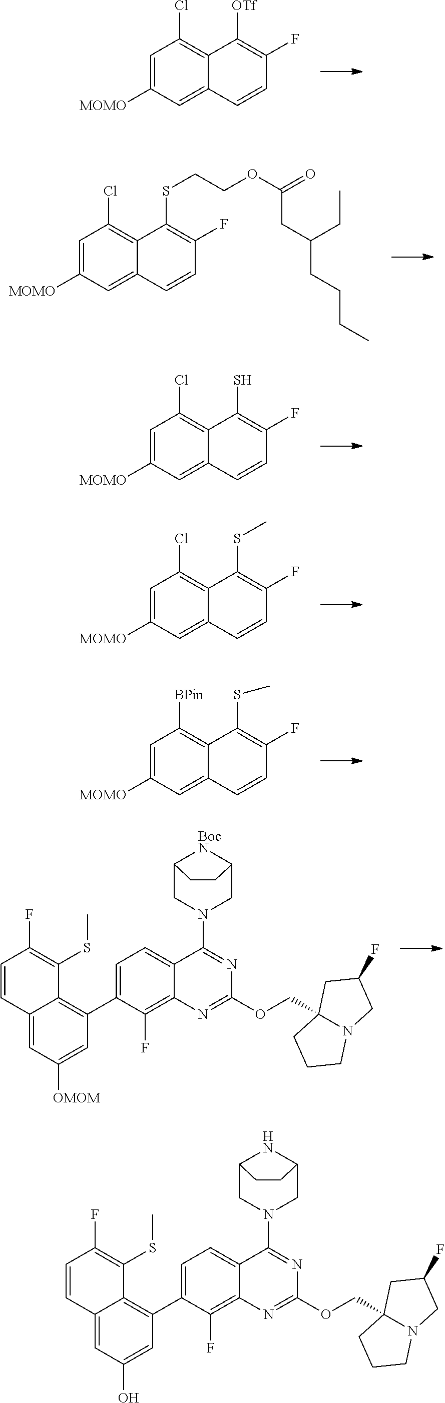

### Step 1: Synthesis of (Z)-6-methoxy-3,4-dihydronaphthalen-1(2H)-one O-methyl oxime

Compound 6-methoxy-3,4-dihydronaphthalen-1(2H)-one (2.1 g, 11.9 mmol), O-methylhydroxylamine hydrochloride (1.19 g, 14.3 mmol) and pyridine (1.41 g, 17.8 mmol) were stirred EtOH (50 mL) at room temperature for 5 h. The mixture was concentrated, diluted with water and extracted with DCM, concentrated to obtain a brown oil, used directly in the next step. ¹HNMR (400 MHz, CDCl₃) δ 7.92 (d, J=8.8 Hz, 1H), 6.75 (dd, J=8.8, 2.7 Hz, 1H), 6.63 (d, J=2.6 Hz, 1H), 3.96 (s, 3H), 3.81 (s, 3H), 2.71 (t, J=6.4 Hz, 4H), 1.88-1.78 (m, 2H).

### Step 2: Synthesis of (Z)-8-chloro-6-methoxy-3,4-dihydronaphthalen-1(2H)-one O-methyl oxime

Compound (Z)-6-methoxy-3,4-dihydronaphthalen-1(2H)-one O-methyl oxime (2.0 g, 9.74 mmol) and NCS (1.56 g, 11.7 mmol) in AcOH (50 mL) was added Pd(OAc)₂ (218 mg, 0.97 mmol) under Ar. The mixture was stirred at 90° C. for 1 h. The solution was poured into ice water, extracted with ethyl acetate, concentrated and separated by column chromatography to obtain a light yellow oil. (2 g, yield: 85.8%). ¹HNMR (400 MHz, CDCl₃) δ 6.86 (d, J=2.6 Hz, 1H), 6.60 (d, J=2.6 Hz, 1H), 4.01 (s, 3H), 3.79 (s, 3H), 2.74 (t, J=6.9 Hz, 2H), 2.64-2.53 (m, 2H), 1.79-1.67 (m, 2H).

### Step 3: Synthesis of 8-chloro-6-methoxy-3,4-dihydronaphthalen-1(2H)-one

Compound (Z)-8-chloro-6-methoxy-3,4-dihydronaphthalen-1(2H)-one O-methyl oxime (7.6 g, 31.7 mmol) was stirred in 1,4-dioxane (100 mL) and 6N HCl (100 mL) at 90° C. for 2 h. The solution was poured into ice water, extracted with ethyl acetate, concentrated and separated by column chromatography to obtain a light yellow solid. (6.0 g, yield: 90.9%). ¹HNMR (400 MHz, CDCl₃) δ 6.86 (d, J=2.6 Hz, 1H), 6.65 (d, J=2.6 Hz, 1H), 3.85 (d, J=6.0 Hz, 3H), 2.93 (t, J=6.1 Hz, 2H), 2.70-2.59 (m, 2H), 2.12-2.01 (m, 2H).

### Step 4: Synthesis of 8-chloro-2-fluoro-6-methoxy-3,4-dihydronaphthalen-1(2H)-one

Compound 8-chloro-6-methoxy-3,4-dihydronaphthalen-1(2H)-one (5.8 g, 27.5 mmol) in MeOH (150 mL) was added Selecetfluor (11.7 g, 33.0 mmol) and concentrated sulfuric acid (0.5 mL). The mixture was stirred at 50° C. for 5 h. The solution was concentrated, diluted with water, extracted with ethyl acetate, concentrated and separated by column chromatography to obtain a light yellow solid. (5.2 g, yield: 82.9%). ¹H NMR (400 MHz, CDCl₃) δ 6.90 (d, J=2.5 Hz, 1H), 6.64 (dd, J=2.5, 1.1 Hz, 1H), 5.04 (ddd, J=48.7, 11.8, 4.9 Hz, 1H), 3.86 (s, 3H), 3.11 (dt, J=6.6, 4.1 Hz, 2H), 2.50 (ddt, J=13.0, 8.3, 4.8 Hz, 1H), 2.41-2.26 (m, 1H).

### Step 5: Synthesis of 2-bromo-8-chloro-2-fluoro-6-methoxy-3,4-dihydronaphthalen-1(2H)-one

Compound 8-chloro-2-fluoro-6-methoxy-3,4-dihydronaphthalen-1(2H)-one (5.2 g, 22.8 mmol) in CH₃CN (100 mL), was added pyridinium tribromide (8.02 g, 25 mmol) under Ar. The mixture was stirred at 60° C. for 1 h. The solution was poured into ice water, extracted with ethyl acetate, concentrated and separated by column chromatography to obtain a light yellow solid. (6.8 g, yield: 97.1%). ¹H NMR (400 MHz, CDCl₃) δ 6.96 (d, J=2.5 Hz, 1H), 6.66 (dd, J=2.4, 1.1 Hz, 1H), 3.32 (ddd, J=17.4, 12.2, 5.0 Hz, 1H), 3.13-3.02 (m, 1H), 2.84-2.75 (m, 1H), 2.66-2.54 (m, 1H).

### Step 6: Synthesis of 8-chloro-2-fluoro-6-methoxynaphthalen-1-ol

Compound 2-bromo-8-chloro-2-fluoro-6-methoxy-3,4-dihydronaphthalen-1(2H)-one (6.6 g, 21.5 mmol) and LiBr (4.1 g, 47.2 mmol) were stirred in DMF (100 mL) at 100° C. for 1 h. The solution was poured into ice water, extracted with ethyl acetate, concentrated and separated by column chromatography to obtain a light yellow solid. (4.6 g, yield: 94.6%). ¹H NMR (400 MHz, CDCl₃) δ 7.30 (dd, J=10.0, 9.0 Hz, 1H), 7.25-7.20 (m, 1H), 7.19-7.16 (m, 1H), 7.01 (d, J=2.5 Hz, 1H), 3.89 (s, 3H).

### Step 7: Synthesis of 8-chloro-2-fluoro-6-methoxynaphthalen-1-yl trifluoromethanesulfonate

Compound 8-chloro-2-fluoro-6-methoxynaphthalen-1-ol (250 mg, 1.1 mmol) in DCM at −60° C. under Ar, was added DIPEA (426 mg, 3.3 mmol) and trifluoromethanesulfonic anhydride (467 mg, 1.65 mmol). The mixture was stirred at room temperature for 10 h. The solution was poured into ice water, extracted with DCM, concentrated and separated by column chromatography to obtain a light yellow solid. (350 mg, yield: 89.7%). ¹H NMR (400 MHz, CDCl₃) δ 7.75 (dd, J=9.2, 5.2 Hz, 1H), 7.42-7.35 (m, 2H), 7.09 (d, J=2.5 Hz, 1H), 3.92 (s, 3H).

### Step 8: Synthesis of 8-chloro-2-fluoro-6-hydroxynaphthalen-1-yl trifluoromethanesulfonate

Compound 8-chloro-2-fluoro-6-methoxynaphthalen-1-yl trifluoromethanesulfonate (50 mg, 0.139 mmol) in DCM (20 mL) was added BBr₃ (0.418 mL, 0.418 mmol) at 0° C. The mixture was stirred at room temperature for 10 h. The solution was poured into ice water, extracted with DCM, concentrated and separated by column chromatography to obtain a light yellow solid. (40 mg, yield: 83.3%). ¹H NMR (400 MHz, CDCl₃) δ 7.70 (dd, J=9.2, 5.2 Hz, 1H), 7.44-7.32 (m, 2H), 7.14 (d, J=2.4 Hz, 1H).

### Step 9: Synthesis of 8-chloro-2-fluoro-6-(methoxymethoxy)naphthalen-1-yl trifluoromethanesulfonate

Compound 8-chloro-2-fluoro-6-hydroxynaphthalen-1-yl trifluoromethanesulfonate (40 mg, 0.116 mmol) in DCM (10 mL) at 0° C., was added DIPEA (44.8 mg, 0.348 mmol) and MOMBr (21.7 mg, 0.174 mmol). The mixture was stirred at room temperature for 10 min. The solution was poured into ice water, extracted with DCM, concentrated and separated by column chromatography to obtain a light yellow solid. (40 mg, yield: 88.9%). ¹H NMR (400 MHz, CDCl₃)) δ 7.77 (dd, J=9.2, 5.2 Hz, 1H), 7.48 (d, J=2.3 Hz, 1H), 7.43-7.33 (m, 2H), 5.28 (s, 2H), 3.52 (s, 3H).

### Step 10: Synthesis of 2-((8-chloro-2-fluoro-6-(methoxymethoxy)naphthalen-1-yl)thio)ethyl 3-ethylheptanoate

Compound 8-chloro-2-fluoro-6-(methoxymethoxy)naphthalen-1-yl trifluoromethanesulfonate (40 mg, 0.102 mmol), 2-mercaptoethyl 3-ethylheptanoate (45 mg, 0.204 mmol), Pd₂(dba)₃ (4.7 mg, 0.005 mmol), Xantphos (5.95 mg, 0.01 mmol) and DIPEA (39.8 mg, 0.308 mmol) were stirred in 1,4-dioxane (10 mL) at 100° C. for 15 h under Ar. The solution was poured into ice water, extracted with ethyl acetate, concentrated and separated by column chromatography to obtain a light yellow solid. (20 mg, yield: 42.5%). ¹H NMR (400 MHz, CDCl₃) δ 7.72 (dd, J=9.0, 5.7 Hz, 1H), 7.47-7.41 (m, 1H), 7.34 (d, J=2.6 Hz, 2H), 5.27 (s, 2H), 3.94 (dd, J=5.9, 2.5 Hz, 2H), 3.52 (s, 3H), 3.14 (t, J=7.4 Hz, 2H), 2.53 (t, J=7.5 Hz, 2H), 1.50 (d, J=5.7 Hz, 1H), 1.25 (m, 8H), 0.88-0.80 (m, 6H).

### Step 11: Synthesis of (8-chloro-2-fluoro-6-(methoxymethoxy)naphthalen-1-yl)(methyl)sulfane

Compound 2-((8-chloro-2-fluoro-6-(methoxymethoxy)naphthalen-1-yl)thio)ethyl 3-ethylheptanoate (100 mg, 0.219 mmol) in THF (10 mL) at 0° C., was added t-BuONa (31.5 mg, 0.328 mmol). The mixture was stirred at room temperature for 1 h, then Mel (31.5 mg, 0.328 mmol) was added at 0° C. The mixture was stirred at room temperature for 2 h. The solution was poured into ice water, extracted with ethyl acetate, concentrated and separated by column chromatography to obtain a light yellow solid. (45 mg, yield: 71.7%).

### Step 12: Synthesis of 2-(7-fluoro-3-(methoxymethoxy)-8-(methylthio)naphthalen-1-yl)-4,4,5,5-tetramethyl-1,3,2-dioxaborolane

Compound (8-chloro-2-fluoro-6-(methoxymethoxy)naphthalen-1-yl)(methyl)sulfane (45 mg, 0.157 mmol), B₂Pin₂ (59.8 g, 0.235 mmol), Pd₂(dba)₃ (14.3 mg, 0.0157 mmol), Sphos (10.4 mg, 0.0254 mmol) and KOAc (46.2 mg, 0.471 mmol) were stirred in 1,4-dioxane (10 mL) at 110° C. for 12 h. After the reaction was complete, the reaction liquid was cooled to room temperature, diluted with water and extracted with ethyl acetate, and the organic phase was concentrated and separated by column chromatography to obtain a white solid. (45 mg, yield: 76.2%).

### Step 13: Synthesis of tert-butyl (1R,5S)-3-(8-fluoro-7-(7-fluoro-3-(methoxymethoxy)-8-(methylthio)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (200 mg, 0.336 mmol), 2-(7-fluoro-3-(methoxymethoxy)-8-(methylthio)naphthalen-1-yl)-4,4,5,5-tetramethyl-1,3,2-dioxaborolane (190 mg, 0.504 mmol), Cs₂CO₃ (328 mg, 1.008 mmol) and Pd(dppf)Cl₂ (24.6 mg, 0.0336 mmol) were stirred in 1,4-dioxane/H₂O (15 mL, 5/1) at 100° C. for 12 h under N₂. After the reaction was complete, the reaction liquid was cooled to room temperature, diluted with water and extracted with ethyl acetate, and the organic phase was dried with anhydrous sodium sulfate, concentrated and separated by prep-TLC to obtain a white solid. (90 mg, yield: 35%). MS m/z: 765.3 [M+H]⁺

### Step 14: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-(methylthio)naphthalen-2-ol

Compound tert-butyl (1R,5S)-3-(8-fluoro-7-(7-fluoro-3-(methoxymethoxy)-8-(methylthio)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (90 mg, 0.117 mmol) was stirred in CH₃CN (3 mL) and HCl/1,4-dioxane (1 mL, 4M) at room temperature for 30 min. After the reaction was complete, pH of the reaction liquid was adjusted to 7 with a saturated sodium bicarbonate aqueous solution and extracted with dichloromethane, and the organic phase was dried with anhydrous sodium sulfate, concentrated, separated and purified by TLC (DCM/MeOH(7N NH₃)=10/1) to obtain a yellow solid. (30 mg, yield: 41%). MS m/z: 622.2 [M+H]⁺

### Example 337: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-(methylsulfonyl)napthalen-2-ol

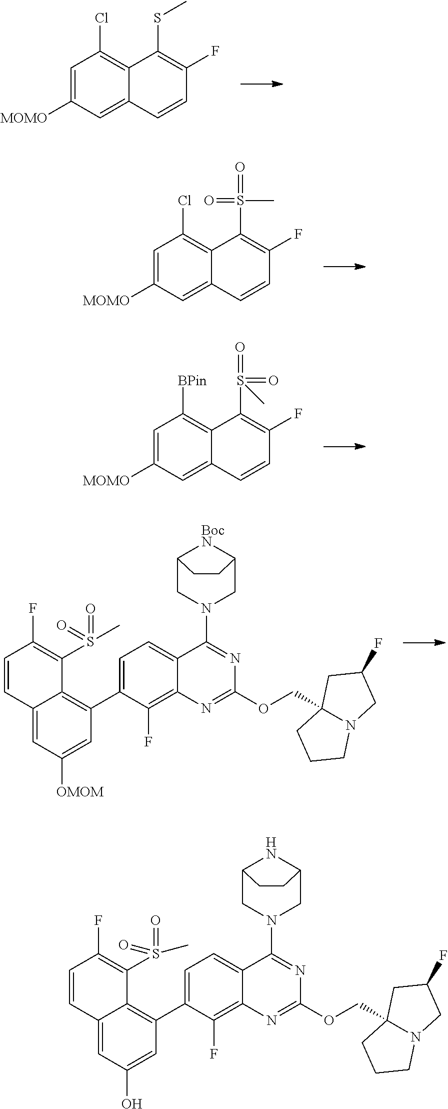

### Step 1: Synthesis of 8-chloro-2-fluoro-6-(methoxymethoxy)-1-(methylsulfonyl)naphthalene

Compound (8-chloro-2-fluoro-6-(methoxymethoxy)naphthalen-1-yl)(methyl)sulfane (200 mg, 0.697 mmol) in DCM (10 mL) at 0° C.-10° C., was added 85% m-CPBA (708 mg, 3.48 mmol). The mixture was stirred at room temperature for 2 h. After the reaction was complete, the reaction was quenched by saturated sodium thiosulfate solution and extracted with dichloromethane, and the organic phase was washed with saturated sodium bicarbonate, dried with anhydrous sodium sulfate and concentrated to obtain a yellow solid, which was directly used for the next step.

### Step 2: Synthesis of 2-(7-fluoro-3-(methoxymethoxy)-8-(methylsulfonyl)naphthalen-1-yl)-4,4,5,5-tetramethyl-1,3,2-dioxaborolane

Compound 8-chloro-2-fluoro-6-(methoxymethoxy)-1-(methylsulfonyl)naphthalene (50 mg, 0.157 mmol), B₂Pin₂ (59.8 g, 0.235 mmol), Pd₂(dba)₃ (14.3 mg, 0.0157 mmol), Sphos (10.4 mg, 0.0254 mmol) and KOAc (46.2 mg, 0.471 mmol) were stirred in 1,4-dioxane (10 mL) under Ar at 110° C. for 12 h. After the reaction was complete, the reaction liquid was cooled to room temperature, diluted with water and extracted with ethyl acetate, concentrated and separated by column chromatography to obtain a white solid. (47 mg, yield: 73.0%).

### Step 3: Synthesis of tert-butyl (1R,5S)-3-(8-fluoro-7-(7-fluoro-3-(methoxymethoxy)-8-(methylsulfonyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (200 mg, 0.336 mmol), 2-(7-fluoro-3-(methoxymethoxy)-8-(methylsulfonyl)naphthalen-1-yl)-4,4,5,5-tetramethyl-1,3,2-dioxaborolane (206.6 mg, 0.504 mmol), Cₛ2CO₃ (328 mg, 1.008 mmol) and Pd(dppf)Cl₂ (24.6 mg, 0.0336 mmol) were stirred in 1,4-dioxane/H₂O (15 mL, 5/1) under N₂ at 100° C. for 12 h. After the reaction was complete, the reaction liquid was cooled to room temperature, diluted with water and extracted with ethyl acetate, and the organic phase was dried with anhydrous sodium sulfate, concentrated and separated by prep-TLC to obtain a white solid. (95 mg, yield: 36.2%). MS m/z: 780.3 [M+H]⁺

### Step 4: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-(methylsulfonyl)naphthalen-2-ol

Compound tert-butyl (1R,5S)-3-(8-fluoro-7-(7-fluoro-3-(methoxymethoxy)-8-(methylsulfonyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (95 mg, 0.122 mmol) was stirred in CH₃CN (3 mL) and 1,4-dioxane (1 mL, 4M) at room temperature for 30 min. After the reaction was complete, pH of the reaction liquid was adjusted to 7 with a saturated sodium bicarbonate aqueous solution and extracted with dichloromethane, and the organic phase was dried with anhydrous sodium sulfate, concentrated, separated and purified by TLC (DCM/MeOH(7N NH₃)=10/1) to obtain a yellow solid. (35 mg, yield: 45.4%). MS m/z: 654.7 [M+H]⁺

### Example 338: Synthesis of (8-(4-((1R,5S)-3,8-diazabicyclo[3.2.1]octan-3-yl)-8-fluoro-2-(((2R,7aS)-2-fluorotetrahvdro-1H-pyrrolizin-7a(5H)-yl)methoxy)quinazolin-7-yl)-2-fluoro-6-hydroxynaphthalen-1-yl)dimethylphosphine oxide

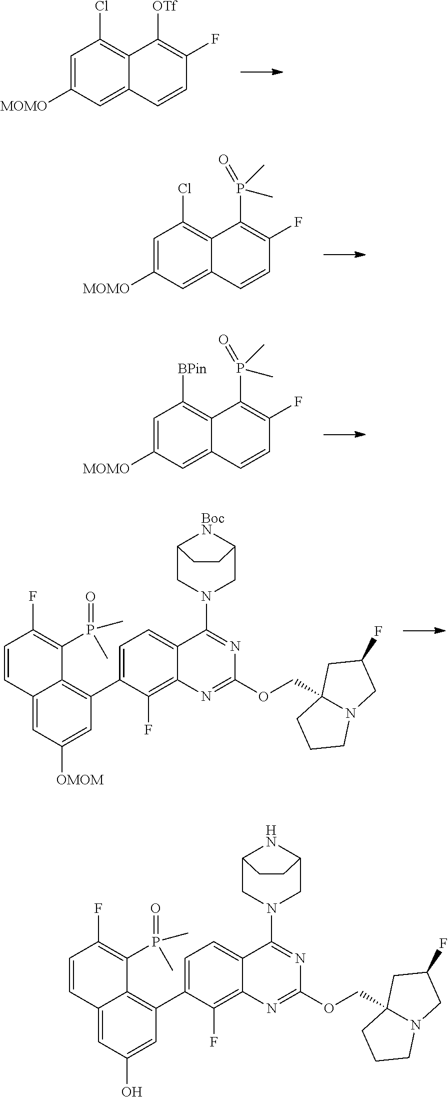

### Step 1: Synthesis of (8-chloro-2-fluoro-6-(methoxymethoxy)naphthalen-1-yl)dimethylphosphine oxide

Compound 8-chloro-2-fluoro-6-(methoxymethoxy)naphthalen-1-yl trifluoromethanesulfonate (40 mg, 0.102 mmol), dimethylphosphine oxide (15.9 mg, 0.204 mmol), Pd₂(dba)₃ (4.7 mg, 0.005 mmol), Xantphos (5.95 mg, 0.01 mmol) and DIPEA (39.8 mg, 0.308 mmol) were stirred in 1,4-dioxane (10 mL) under Ar at 100° C. for 15 h. The solution was poured into ice water, extracted with ethyl acetate, concentrated and separated by column chromatography to obtain a light yellow solid. (25 mg, yield: 78.1%).

### Step 2: Synthesis of (2-fluoro-6-(methoxymethoxy)-8-(4,4,5,5-tetramethyl-1,3,2-dioxaborolan-2-yl)naphthalen-1-yl)dimethylphosphine oxide

Compound (8-chloro-2-fluoro-6-(methoxymethoxy)naphthalen-1-yl)dimethylphosphine oxide (49.7 mg, 0.157 mmol), B₂Pin₂ (59.8 g, 0.235 mmol), Pd₂(dba)₃ (14.3 mg, 0.0157 mmol), Sphos (10.4 mg, 0.0254 mmol) and KOAc (46.2 mg, 0.471 mmol) were stirred in 1,4-dioxane (10 mL) under Ar at 110° C. for 12 h. After the reaction was complete, the reaction liquid was cooled to room temperature, diluted with water and extracted with ethyl acetate, concentrated and separated by column chromatography to obtain a white solid. (48 mg, yield: 75.0%).

### Step 3: Synthesis of tert-butyl (1R,5S)-3-(7-(8-(dimethylphosphoryl)-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (200 mg, 0.336 mmol), (2-fluoro-6-(methoxymethoxy)-8-(4,4,5,5-tetramethyl-1,3,2-dioxaborolan-2-yl)naphthalen-1-yl)dimethylphosphine oxide (205.6 mg, 0.504 mmol), Cs₂CO₃ (328 mg, 1.008 mmol) and Pd(dppf)Cl₂ (24.6 mg, 0.0336 mmol) were stirred in 1,4-dioxane/H₂O (15 mL, 5/1) under N₂ at 100° C. for 12 h. After the reaction was complete, the reaction liquid was cooled to room temperature, diluted with water and extracted with ethyl acetate, and the organic phase was dried with anhydrous sodium sulfate, concentrated and separated by prep-TLC to obtain a white solid. (98 mg, yield: 36.7%). MS m/z: 796.3 [M+H]⁺

### Step 4: Synthesis of (8-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-2-fluoro-6-hydroxynaphthalen-1-yl)dimethylphosphine oxide

Compound tert-butyl (1R,5S)-3-(7-(8-(dimethylphosphoryl)-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (98 mg, 0.123 mmol) was stirred in CH₃CN (3 mL) and HCl/1,4-dioxane (1 mL, 4M) at room temperature for 30 min. After the reaction was complete, pH of the reaction liquid was adjusted to 7 with a saturated sodium bicarbonate aqueous solution and extracted with dichloromethane, and the organic phase was dried with anhydrous sodium sulfate, concentrated, separated and purified by TLC (DCM/MeOH(7N NH₃)=10/1) to obtain a yellow solid. (35 mg, yield: 43.7%). MS m/z: 652.2 [M+H]⁺

### Example 339: Synthesis of 8-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-ethynylisoquinolin-6-ol

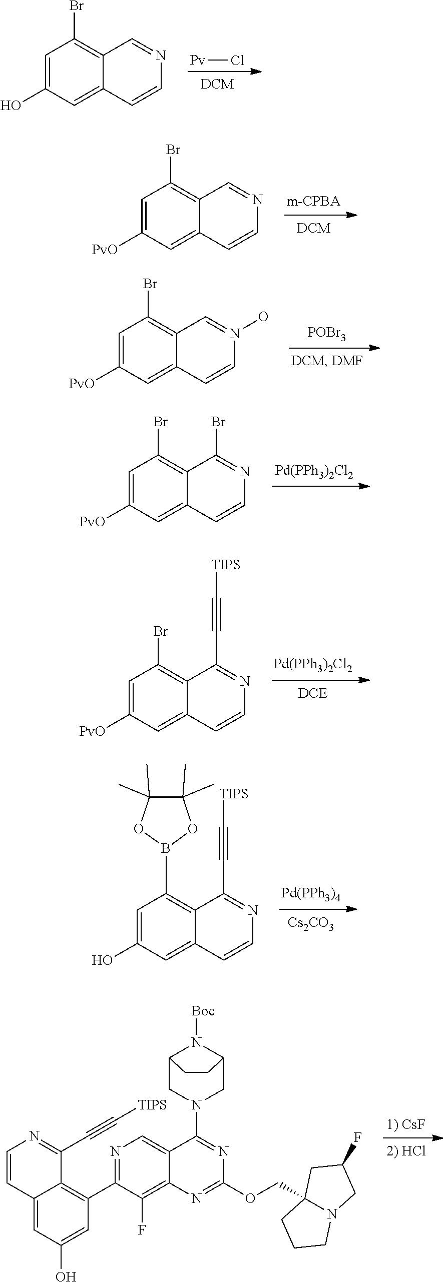

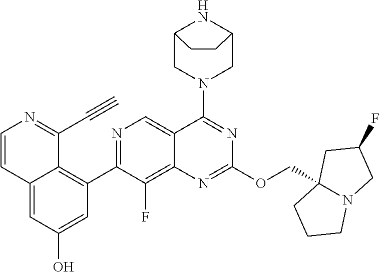 [SMILES: C#Cc1nccc2cc(O)cc(-c3ncc4c(N5CC6CCC(C5)N6)nc(OC[C@@]56CCCN5C[C@H](F)C6)nc4c3F)c12]

### Step 1: Synthesis of 8-bromoisoquinolin-6-yl pivalate

Compound 8-bromoisoquinolin-6-ol (1.13 g, 5.04 mmol) and DIEA (1.3 g, 10.08 mmol) in DCM (20 mL) at 0° C., was added pivaloyl chloride (608 mg, 5.04 mmol) dropwise. The mixture was stirred at 0° C. for 15 min. After the reaction was complete, the mixture was concentrated, diluted with water and extracted with ethyl acetate, and the organic phase was washed with sat. NaCl, dried with anhydrous sodium sulfate, concentrated to obtain a yellow oil. (1.32 g, yield: 84.9%). ¹H NMR (400 MHz, CDCl₃) δ 9.62 (s, 1H), 8.60 (d, J=5.4 Hz, 1H), 7.72 (d, J=5.8 Hz, 1H), 7.69 (d, J=1.8 Hz, 1H), 7.62 (s, 1H), 1.41 (s, 9H).

### Step 2: Synthesis of 8-bromo-6-(pivaloyloxy)isoquinoline 2-oxide

Compound 8-bromoisoquinolin-6-yl pivalate (1.32 g, 4.28 mmol) in DCM (30 mL) at 0° C., was added 85% m-CPBA (2.18 g, 10.71 mmol) portionwise. The mixture was stirred at room temperature for 1 h. After the reaction was complete, the reaction was quenched by saturated sodium sulfite solution and extracted with dichloromethane, and the organic phase was washed with saturated sodium bicarbonate and brine, dried with anhydrous sodium sulfate and concentrated to obtain a white solid. (1.4 g, yield: 100%). ¹H NMR (400 MHz, CDCl₃) δ 9.23 (s, 1H), 8.26 (d, J=6.6 Hz, 1H), 7.72-7.66 (m, 2H), 7.58 (s, 1H), 1.40 (s, 9H).

### Step 3: Synthesis of 1,8-dibromoisoquinolin-6-yl pivalate

Compound 8-bromo-6-(pivaloyloxy)isoquinoline 2-oxide (1.4 g, 4.31 mmol) in DCM (40 mL) at 0° C., was added POBr₃ (3.71 g, 12.96 mmol) and DMF (0.5 mL, 6.46 mmol) dropwise. The mixture was stirred at 0° C. for 30 min. After the reaction was complete, the solution was poured into saturated sodium bicarbonate and extracted with dichloromethane, and the organic phase was dried with anhydrous sodium sulfate, concentrated and separated by column chromatography (PE/EA=20/1) to obtain a yellow solid. (360 mg, yield: 21.6%). ¹H NMR (400 MHz, CDCl₃) δ 8.31 (d, J=4.6 Hz, 1H), 7.83 (dd, J=15.6, 2.2 Hz, 1H), 7.70-7.58 (m, 2H), 1.40 (s, 9H).

### Step 4: Synthesis of 8-bromo-1-((triisopropylsilyl)ethynyl)isoquinolin-6-yl pivalate

Compound 1,8-dibromoisoquinolin-6-yl pivalate (100 mg, 0.26 mmol) in THF/iPr₂NH=1/1(5 mL) was added CuI (3 mg, 0.013 mmol), Pd(PPh₃)₂Cl₂ (4 mg, 0.005 mmol) and (triisopropylsilyl)acetylene (52 mg, 0.28 mmol). The mixture was stirred at 60° C. for 2 h under N₂. After the reaction was complete, the solution was concentrated and separated by prep-TLC (PE/EA=10/1) to obtain a white solid. (65 mg, yield: 51.5%). ¹H NMR (400 MHz, CDCl₃) δ 8.57 (d, J=4.6 Hz, 1H), 7.77 (s, 1H), 7.71 (d, J=4.6 Hz, 1H), 7.64 (s, 1H), 1.40 (s, 9H), 1.21 (d, J=6.5 Hz, 21H).

### Step 5: Synthesis of 8-(4,4,5,5-tetramethyl-1,3,2-dioxaborolan-2-yl)-1-((triisopropylsilyl)ethynyl)isoquinolin-6-ol

Compound 8-bromo-1-((triisopropylsilyl)ethynyl)isoquinolin-6-yl pivalate (30 mg, 0.06 mmol), Pd(PPh₃)₂Cl₂ (3 mg, 0.003 mmol), TEA (31 mg, 0.307 mmol) and pinacolborane (24 mg, 0.184 mmol) were stirred in dichloroethane (5 mL) at 90° C. for 6 h under N₂. After the reaction was complete, the solution was concentrated and separated by prep-TLC (PE/EA=10/1) to obtain a yellow solid. (18 mg, yield: 65.3%). ¹H NMR (400 MHz, CDCl₃) δ 8.59 (d, J=5.3 Hz, 1H), 7.52 (s, 1H), 7.40 (s, 1H), 7.36 (s, 1H), 1.42 (s, 12H), 1.16 (d, J=7.4 Hz, 21H).

### Step 6: Synthesis of tert-butyl (1R,5S)-3-(8-fluoro-2-(((2R,7aS)-2-fluorotetrahydro-1H-pyrrolizin-7a(5H)-yl)methoxy)-7-(6-hydroxy-1-((triisopropylsilyl)ethynyl)isoquinolin-8-yl)pyrido-[4,3-d]pyrimidin-4-yl)-3,8-diazabicyclo[3.2.1]octane-8-carboxylate

Compound tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (14 mg, 0.025 mmol), 8-(4,4,5,5-tetramethyl-1,3,2-dioxaborolan-2-yl)-1-((triisopropylsilyl)ethynyl)isoquinolin-6-ol (15 mg, 0.033 mmol), Cs₂CO₃ (16 mg, 0.05 mmol) and Pd(PPh₃)₄(14 mg, 0.013 mmol) were stirred in 1,4-dioxane/H₂O (3 mL, 511) at 100° C. for 4 h under N₂. After the reaction was complete, the reaction liquid was cooled to room temperature, diluted with water and extracted with ethyl acetate, and the organic phase was dried with anhydrous sodium sulfate, concentrated and separated by prep-TLC (DCM/MeOH=1511) to obtain a white solid. (17 mg, yield: 79%).

### Step 7: Synthesis of 8-(4-((1R,5S)-3,8-diazabicyclo[3.2.1]octan-3-)-8-fluoro-2-(((2R,7aS)-2-fluorotetrahydro-1H-pyrrolizin-7a(5H)-yl)methoxy)pyrido[4,3-d]pyrimidin-7-yl)-1-ethynylisoquinolin-6-ol

Compound tert-butyl (1R,5S)-3-(8-fluoro-2-(((2R,7aS)-2-fluorotetrahydro-H-pyrrolizin-7a(5H)-yl)methoxy)-7-(6-hydroxy-1-((triisopropylsilyl)ethynyl)isoquinolin-8-yl)pyrido[4,3-d]pyrimidin-4-yl)-3,8-diazabicyclo[3.2.1]octane-8-carboxylate (16 mg, 0.019 mmol) and CsF (15 mg, 0.095 mmol) were stirred in DMF (3 mL) at room temperature for 4 h. After the reaction was complete detected by TLC, the reaction liquid was diluted with brine and extracted with ethyl acetate, and the organic phase was dried with anhydrous sodium sulfate, concentrated to obtain a white solid. Then, the solid was stirred in CH₃CN (3 mL) and HCl/1,4-dioxane (1 mL, 4N) at room temperature for 30 min. After the reaction was complete, pH of the solution was adjusted to 7-8 with saturated sodium bicarbonate aqueous solution, extracted with ethyl acetate, and the organic phase was dried with anhydrous sodium sulfate, concentrated, purified by prep-TLC (DCM/MeOH(7N NH₃)=15/1) to give a white solid (7 mg, yield: 62.9%). MS m/z: 584.6 [M+H]⁺

Examples 328-350 were obtained by using the method of Example 327.

| | | | m/z: ES⁺ |
|---|---|---|---|
| Ex. | Compound name | Structural formula | [M + H]⁺ |
| 340 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- ethylisoquinolin-6-ol | 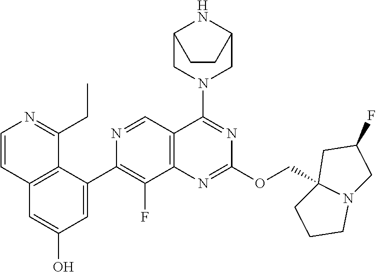 [SMILES: CCc1nccc2cc(O)cc(-c3ncc4c(N5CC6CCC(C5)N6)nc(OC[C@@]56CCCN5C[C@H](F)C6)nc4c3F)c12] | 588.6 |
| 341 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- (methylthio)isoquinolin-6-ol | 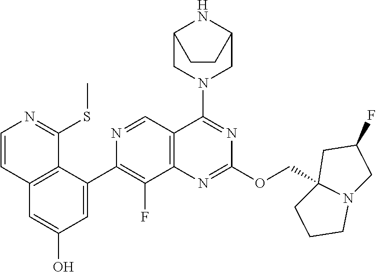 [SMILES: CSc1nccc2cc(O)cc(-c3ncc4c(N5CC6CCC(C5)N6)nc(OC[C@@]56CCCN5C[C@H](F)C6)nc4c3F)c12] | 605.2 |
| 342 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- methoxyisoquinolin-6-ol | 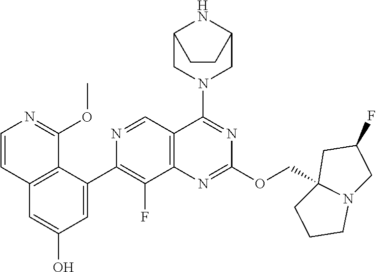 [SMILES: COc1nccc2cc(O)cc(-c3ncc4c(N5CC6CCC(C5)N6)nc(OC[C@@]56CCCN5C[C@H](F)C6)nc4c3F)c12] | 590.2 |
| 343 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- methylisoquinolin-6-ol | 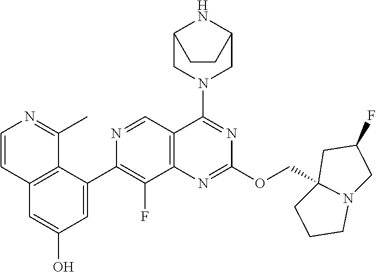 [SMILES: Cc1nccc2cc(O)cc(-c3ncc4c(N5CC6CCC(C5)N6)nc(OC[C@@]56CCCN5C[C@H](F)C6)nc4c3F)c12] | 574.6 |
| 344 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-1- ethylisoquinolin-6-ol | 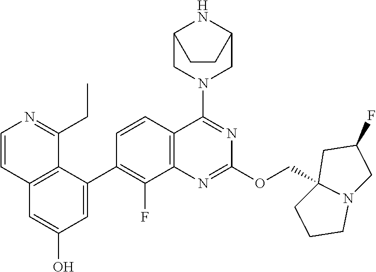 [SMILES: CCc1nccc2cc(O)cc(-c3ccc4c(N5CC6CCC(C5)N6)nc(OC[C@@]56CCCN5C[C@H](F)C6)nc4c3F)c12] | 587.7 |
| 345 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-1- ethynylisoquinolin-6-ol | 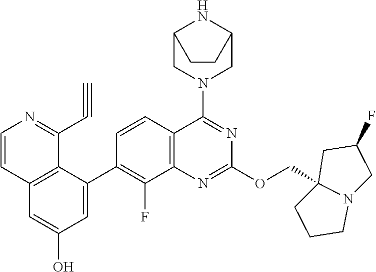 [SMILES: C#Cc1nccc2cc(O)cc(-c3ccc4c(N5CC6CCC(C5)N6)nc(OC[C@@]56CCCN5C[C@H](F)C6)nc4c3F)c12] | 583.7 |
| 346 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-1- (methylthio)isoquinolin-6-ol | 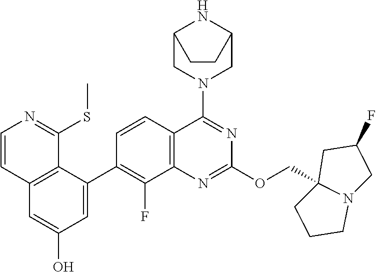 [SMILES: CSc1nccc2cc(O)cc(-c3ccc4c(N5CC6CCC(C5)N6)nc(OC[C@@]56CCCN5C[C@H](F)C6)nc4c3F)c12] | 605.7 |
| 347 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)quinazolin-7-yl)-1- methoxyisoquinolin-6-ol | 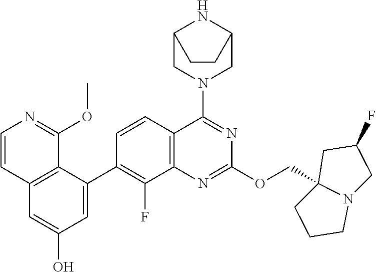 [SMILES: COc1nccc2cc(O)cc(-c3ccc4c(N5CC6CCC(C5)N6)nc(OC[C@@]56CCCN5C[C@H](F)C6)nc4c3F)c12] | 589.6 |
| 348 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-3- ethylbenzo[b]thiophen-6-ol | 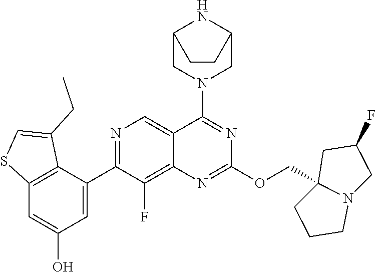 [SMILES: CCc1csc2cc(O)cc(-c3ncc4c(N5CC6CCC(C5)N6)nc(OC[C@@]56CCCN5C[C@H](F)C6)nc4c3F)c12] | 593.2 |
| 349 | 5-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-3- ethylimidazo[1,5-a]pyridin-7-ol | 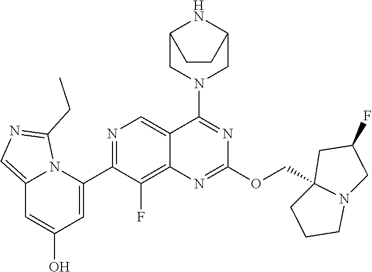 [SMILES: CCc1ncc2cc(O)cc(-c3ncc4c(N5CC6CCC(C5)N6)nc(OC[C@@]56CCCN5C[C@H](F)C6)nc4c3F)n12] | 577.2 |
| 350 | 5-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-3- ethylimidazo[1,2-a]pyridin-7-ol | 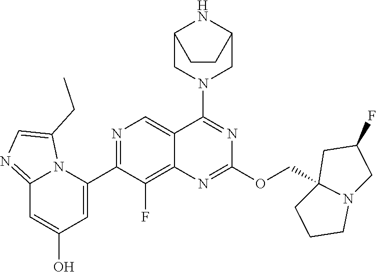 [SMILES: CCc1cnc2cc(O)cc(-c3ncc4c(N5CC6CCC(C5)N6)nc(OC[C@@]56CCCN5C[C@H](F)C6)nc4c3F)n12] | 577.2 |
| 351 | 5-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-3- ethylindolizin-7-ol | 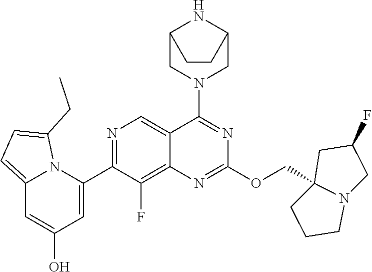 [SMILES: CCc1ccc2cc(O)cc(-c3ncc4c(N5CC6CCC(C5)N6)nc(OC[C@@]56CCCN5C[C@H](F)C6)nc4c3F)n12] | 576.2 |
| 352 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- ethylindolizin-6-ol | 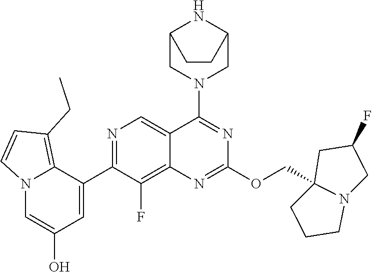 [SMILES: CCc1ccn2cc(O)cc(-c3ncc4c(N5CC6CCC(C5)N6)nc(OC[C@@]56CCCN5C[C@H](F)C6)nc4c3F)c12] | 576.2 |
| 353 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- ethylimidazo[1,5-a]pyridin-6-ol | 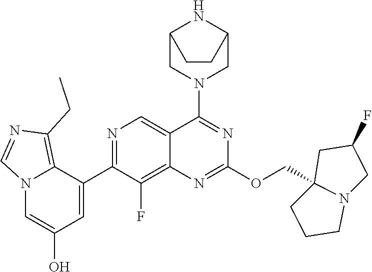 [SMILES: CCc1ncn2cc(O)cc(-c3ncc4c(N5CC6CCC(C5)N6)nc(OC[C@@]56CCCN5C[C@H](F)C6)nc4c3F)c12] | 577.2 |
| 354 | 7-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- (methylthio)-2,3-dihydro-1H-inden-5-ol | 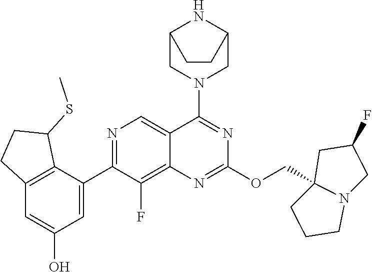 [SMILES: CSC1CCc2cc(O)cc(-c3ncc4c(N5CC6CCC(C5)N6)nc(OC[C@@]56CCCN5C[C@H](F)C6)nc4c3F)c21] | 595.2 |
| 355 | 5-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-6- ethyl-1,1a,6,6a-tetrahydrocyclopropa[a]inden-3-ol | 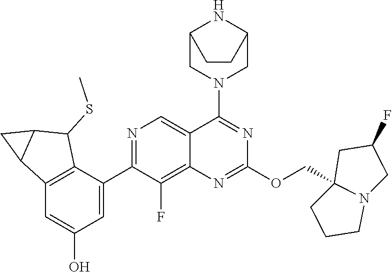 [SMILES: CSC1c2c(-c3ncc4c(N5CC6CCC(C5)N6)nc(OC[C@@]56CCCN5C[C@H](F)C6)nc4c3F)cc(O)cc2C2CC21] | 589.7 |
| 356 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- methylisochroman-6-ol | 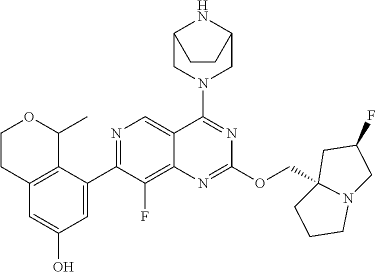 [SMILES: CC1OCCc2cc(O)cc(-c3ncc4c(N5CC6CCC(C5)N6)nc(OC[C@@]56CCCN5C[C@H](F)C6)nc4c3F)c21] | 579.7 |
| 357 | tert-butyl (3-((1R,5S)-3-(8-fluoro-2-(((2R,7aS)-2- fluorotetrahydro-1H-pyrrolizin-7a(5H)-yl)methoxy)-7- (3-hydroxy-8-methyl-5,6,7,8-tetrahydronaphthalen-1- yl)pyrido[4,3-d]pyrimidin-4-yl)-3,8- diazabicyclo[3.2.1]octan-8-yl)-2- hydroxypropyl)carbamate | 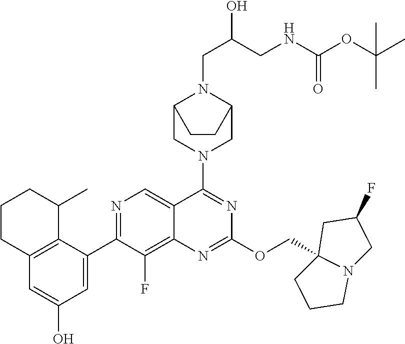 [SMILES: CC1CCCc2cc(O)cc(-c3ncc4c(N5CC6CCC(C5)N6CC(O)CNC(=O)OC(C)(C)C)nc(OC[C@@]56CCCN5C[C@H](F)C6)nc4c3F)c21] | 750.4 |
| 358 | N-(3-((1R,5S)-3-(8-fluoro-2-(((2R,7aS)-2- fluorotetrahydro-1H-pyrrolizin-7a(5H)-yl)methoxy)-7- (3-hydroxy-8-methyl-5,6,7,8-tetrahydronaphthalen-1- yl)pyrido[4,3-d]pyrimidin-4-yl)-3,8- diazabicyclo[3.2.1]octan-8-yl)-2- hydroxypropyl)acetamide | 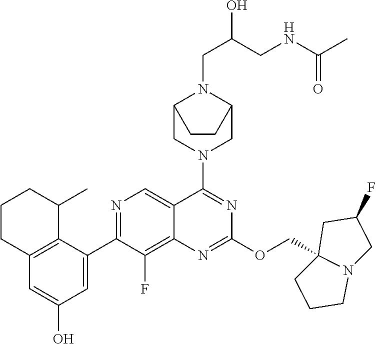 [SMILES: CC(=O)NCC(O)CN1C2CCC1CN(c1nc(OC[C@@]34CCCN3C[C@H](F)C4)nc3c(F)c(-c4cc(O)cc5c4C(C)CCC5)ncc13)C2] | 692.8 |
| 359 | 8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-1- methyl-1,2,3,4-tetrahydroquinolin-6-ol | 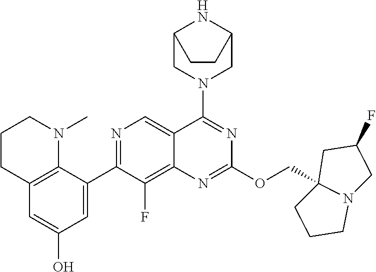 [SMILES: CN1CCCc2cc(O)cc(-c3ncc4c(N5CC6CCC(C5)N6)nc(OC[C@@]56CCCN5C[C@H](F)C6)nc4c3F)c21] | 578.6 |
| 360 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-6- fluoro-5-(methylthio)naphthalen-2-ol | 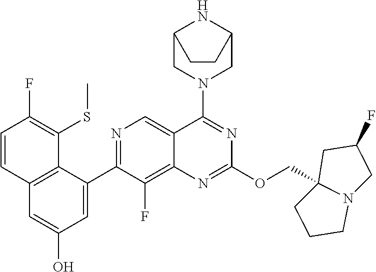 [SMILES: CSc1c(F)ccc2cc(O)cc(-c3ncc4c(N5CC6CCC(C5)N6)nc(OC[C@@]56CCCN5C[C@H](F)C6)nc4c3F)c12] | 623.7 |
| 361 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-6- fluoro-5-(methylsulfinyl)naphthalen-2-ol | 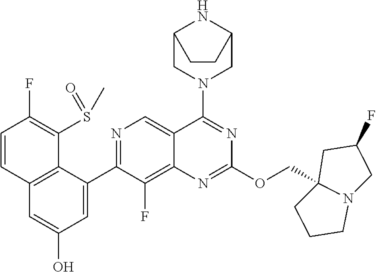 [SMILES: CS(=O)c1c(F)ccc2cc(O)cc(-c3ncc4c(N5CC6CCC(C5)N6)nc(OC[C@@]56CCCN5C[C@H](F)C6)nc4c3F)c12] | 639.4 |
| 362 | (8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]pyrimidin-7-yl)-2- fluoro-6-hydroxynaphthalen-1-yl)dimethylphosphine oxide | 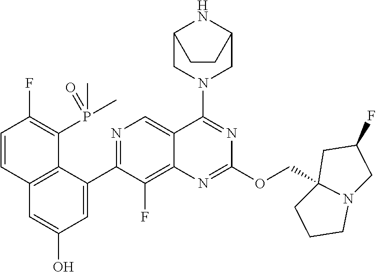 [SMILES: CP(C)(=O)c1c(F)ccc2cc(O)cc(-c3ncc4c(N5CC6CCC(C5)N6)nc(OC[C@@]56CCCN5C[C@H](F)C6)nc4c3F)c12] | 653.6 |

### Example 363: Synthesis of 4-((4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)ethynyl)naphthalen-2-ol

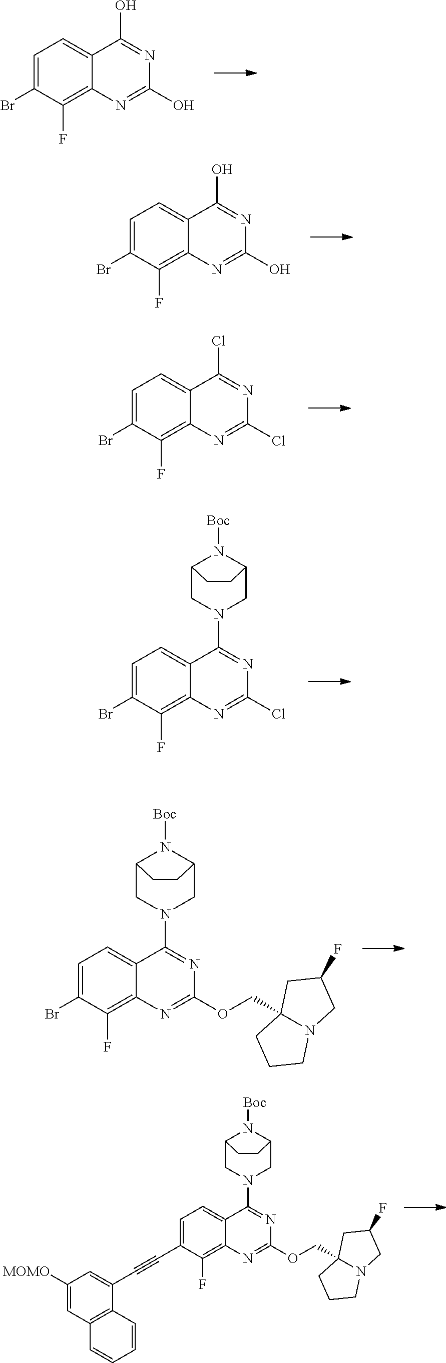

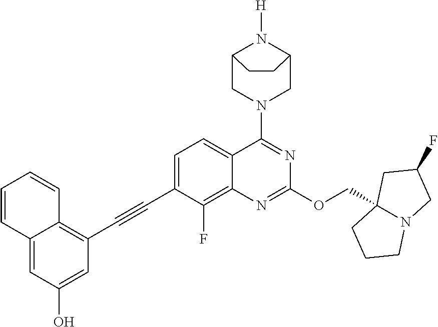 [SMILES: Oc1cc(C#Cc2ccc3c(N4CC5CCC(C4)N5)nc(OC[C@@]45CCCN4C[C@H](F)C5)nc3c2F)c2ccccc2c1]

### Step 1: Synthesis of 7-bromo-2,4-dichloro-8-fluoroquinazoline

Compound 7-bromo-8-fluoroquinazoline-2,4-diol (4.3 g, 20 mmol) in POCl₃ (50 ml) was added N,N-diethylaniline (5 ml) at room temperature. The resulting suspension was stirred at 110° C. overnight. After the reaction was complete, the reaction liquid was concentrated and purified by column chromatography (petroleum ether) to obtain a yellow solid. (3.8 g, yield: 75%). ¹HNMR (400 MHz, CDCl₃) δ 8.12 (s, 1H), 7.93 (s, 1H).

### Step 2: Synthesis of tert-butyl (1R,5S)-3-(7-bromo-2-chloro-8-fluoroquinazolin-4-yl)-3,8-diazabicyclo[3.2.1]octane-8-carboxylate

Compound 7-bromo-2,4-dichloro-8-fluoroquinazoline (1.0 g, 3.38 mmol) and tert-butyl (1R,5S)-3,8-diazabicyclo[3.2.1]octane-8-carboxylate (3.5 g, 13.9 mmol) in DMF (40 mL) at 0° C. was added DIEA (4.6 mL, 27.8 mmol). The resulting suspension was stirred at 0° C. for 30 min. After the reaction was complete, the reaction liquid was diluted with water and extracted with ethyl acetate, and the organic phase was washed with a saturated aqueous NaCl solution, dried with anhydrous sodium sulfate, concentrated to obtain a light yellow solid, washed with petroleum ether, filtered and dried in vacuum to obtain a light yellow solid. (5.3 g, yield: 90%). ¹H NMR (400 MHz, CDCl₃) δ 7.57-7.42 (m, 2H), 4.37 (s, 4H), 3.68 (d, J=71.0 Hz, 2H), 1.99-1.88 (m, 2H), 1.73 (s, 2H), 1.52 (s, 9H).

### Step 3: Synthesis of tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

The compound ((2R,7aS)-2-fluorotetrahydro-1H-pyrrolizin-7a(5H)-yl)methanol (1.45 g, 9.1 mmol) was dissolved in anhydrous tetrahydrofuran (50 mL), 60% NaH (336 mg, 8.4 mmol) was added under ice-water bath cooling condition and reacted under stirring for 30 min, and tert-butyl (1R,5S)-3-(7-bromo-2-chloro-8-fluoroquinazolin-4-yl)-3,8-diazabicyclo[3.2.1]octane-8-carboxylate (3.0 g, 7 mmol) was added and reacted under stirring for 4 h. After the reaction was complete, the reaction was quenched with cold water, extracted with ethyl acetate, and the organic phase was washed with a saturated aqueous NaCl solution, dried with anhydrous sodium sulfate, concentrated and separated by column chromatography to obtain an off-white solid. (2.9 g, yield: 41%).

### Step 4: Synthesis of tert-butyl (1R,5S)-3-(8-fluoro-2-(((2R,7aS)-2-fluorotetrahydro-1H-pyrrolizin-7a(5H)-yl)methoxy)-7-((3-(methoxymethoxy)naphthalen-1-yl)ethynyl)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (53 mg, 0.1 mmol), ethynyl-3-(methoxymethoxy) naphthalene (60 mg, 0.25 mmol), CuI (4 mg, 0.02 mmol) and Pd(PPh₃)₄ were stirred in TEA/THF (2 ml/2 ml) under Ar at 75° C. overnight. After the reaction was complete, the mixture was concentrated and separated by prep-TLC to obtain an yellow solid. (35 mg, yield: 20%). MS m/z: 726 [M+H]⁺.

### Step 5: Synthesis of 4-((4-((1R,5S)-3,8-diazabicyclo[3.2.1]octan-3-yl)-8-fluoro-2-((2R,7aS)-2-fluorotetrahydro-1H-pyrrolizin-7a(5H)-yl)methoxy)quinazolin-7-yl)ethynyl)naphthalen-2-ol

Compound tert-butyl (1R,5S)-3-(8-fluoro-2-(((2R,7aS)-2-fluorotetrahydro-H-pyrrolizin-7a(5H)-yl)methoxy)-7-((3-(methoxymethoxy)naphthalen-1-yl)ethynyl)quinazolin-4-yl)-3,8-diazabicyclo[3.2.1]octane-8-carboxylate was stirred in HCl/1,4-dioxane (5 mL, 4M) at room temperature for 2h. After the reaction was complete, the mixture was concentrated, diluted with saturated sodium bicarbonate aqueous solution and extracted with dichloromethane, and the organic phase was dried with anhydrous sodium sulfate, concentrated, separated and purified by prep-TLC to obtain a yellow solid. (25 mg, yield: 48%). MS m/z: 582 [M+H]+.

Examples 352-356 were obtained by using the method of Example 351

| | | | m/z: ES⁺ |
|---|---|---|---|
| Ex. | Compound name | Structural formula | [M + H] |
| 364 | 8-((4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)quinazolin-7- yl)ethynyl)naphthalen-1-ol | 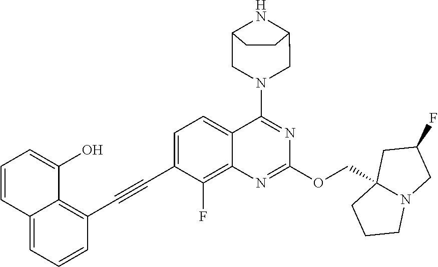 [SMILES: Oc1cccc2cccc(C#Cc3ccc4c(N5CC6CCC(C5)N6)nc(OC[C@@]56CCCN5C[C@H](F)C6)nc4c3F)c12] | 582 |
| 365 | 1-((4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)quinazolin-7- yl)ethynyl)naphthalen-2-ol | 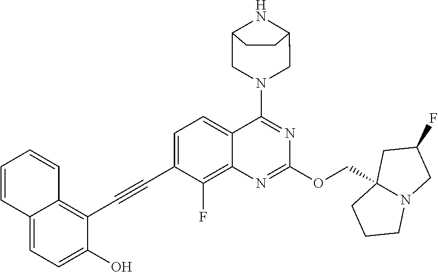 [SMILES: Oc1ccc2ccccc2c1C#Cc1ccc2c(N3CC4CCC(C3)N4)nc(OC[C@@]34CCCN3C[C@H](F)C4)nc2c1F] | 582 |
| 366 | 8-((4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)quinazolin-7-yl)ethynyl)-7- fluoronaphthalene-1,3-diol | 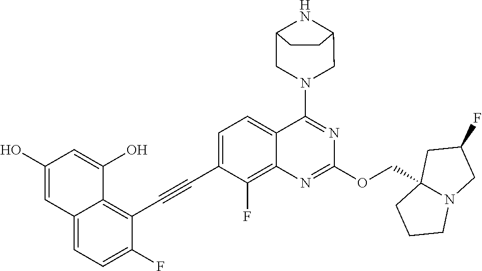 [SMILES: Oc1cc(O)c2c(C#Cc3ccc4c(N5CC6CCC(C5)N6)nc(OC[C@@]56CCCN5C[C@H](F)C6)nc4c3F)c(F)ccc2c1] | 616 |
| 367 | 5-((4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)quinazolin-7-yl)ethynyl)-6- fluoronaphthalen-2-ol | 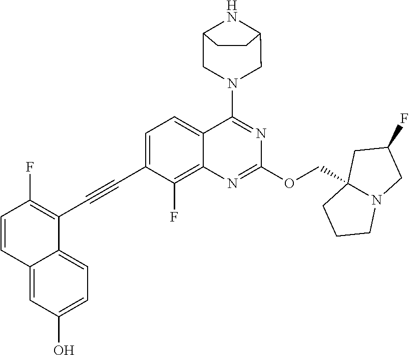 [SMILES: Oc1ccc2c(C#Cc3ccc4c(N5CC6CCC(C5)N6)nc(OC[C@@]56CCCN5C[C@H](F)C6)nc4c3F)c(F)ccc2c1] | 600.6 |
| 368 | 5-((4-((1R,5S)-3,8-diazabicyclo[3.2.1]octan- 3-yl)-8-fluoro-2-(((2R,7aS)-2- fluorotetrahydro-1H-pyrrolizin-7a(5H)- yl)methoxy)pyrido[4,3-d]pyrimidin-7- yl)ethynyl)-6-fluoronaphthalen-2-ol | 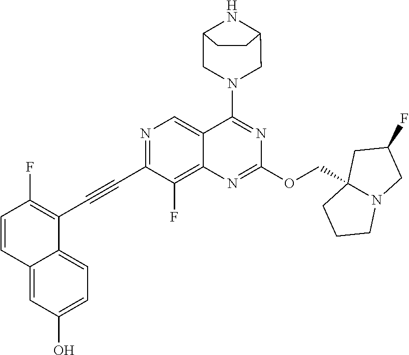 [SMILES: Oc1ccc2c(C#Cc3ncc4c(N5CC6CCC(C5)N6)nc(OC[C@@]56CCCN5C[C@H](F)C6)nc4c3F)c(F)ccc2c1] | 601.6 |

### Example 369: Synthesis of 4-(4-((1R,5S)-3,8-diazabicyclo[3.2.1]octan-3-yl)-6-ethynyl-8-fluoro-2-(((2R,7aS)-2-fluorotetrahydro-1H-pyrrolizin-7a(5H)-yl)methoxy)quinazolin-7-yl)naphthalen-2-ol

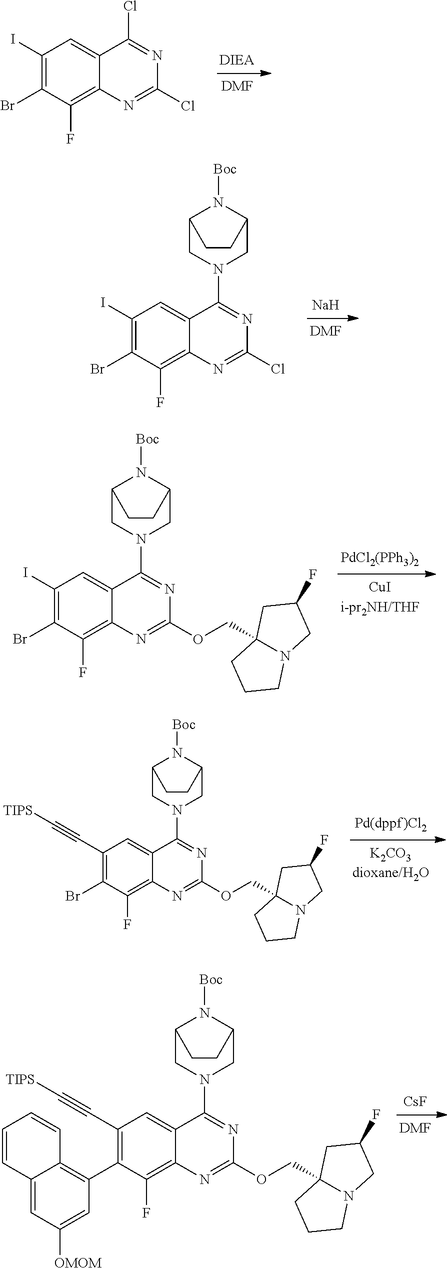

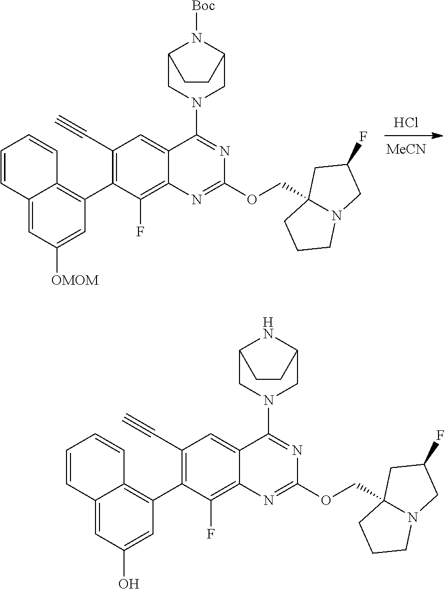

### Step 1: Synthesis of tert-butyl (1R,5S)-3-(7-bromo-2-chloro-8-fluoro-6-iodoquinazolin-4-yl)-3,8-diazabicyclo[3.2.1]octane-8-carboxylate

The compound 7-bromo-2,4-dichloro-8-fluoro-6-iodoquinazoline (1.0 g, 2.37 mmol) was dissolved in DMF (10 mL), DIEA (460 mg, 3.55 mmol) and tert-butyl (1R,5S)-3,8-diazabicyclo[3.2.1]octane-8-carboxylate (553 mg, 2.60 mmol) were added under ice-water bath cooling condition. After stirring for 10 min under ice-water bath cooling condition, the reaction liquid was diluted with brine, filtered and the solid was washed with water, dried to give a yellow solid. (1.4 g, yield: 98%). ¹H NMR (400 MHz, DMSO) δ 8.34 (s, 1H), 4.36 (d, J=12.5 Hz, 2H), 4.24 (s, 2H), 3.62 (d, J=12.4 Hz, 2H), 1.77 (s, 2H), 1.60 (d, J=7.6 Hz, 2H), 1.46 (s, 9H).

### Step 2: Synthesis of tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate

The compound ((2R,7aS)-2-fluorotetrahydro-1H-pyrrolizin-7a(5H)-yl)methanol (484 mg, 3.04 mmol) was dissolved in anhydrous tetrahydrofuran (15 mL), 60% NaH (113 mg, 2.81 mmol) was added under ice-water bath cooling condition and reacted at 0° C. under stirring for 20 min, and tert-butyl (1R,5S)-3-(7-bromo-2-chloro-8-fluoro-6-iodoquinazolin-4-yl)-3,8-diazabicyclo[3.2.1]octane-8-carboxylate (1.4 g, 2.34 mmol) was added and reacted at room temperature under stirring for 8 h. After the reaction was complete, the reaction was quenched with cold water, extracted with ethyl acetate, and the organic phase was washed with a saturated aqueous NaCl solution, dried with anhydrous sodium sulfate, concentrated and separated by column chromatography to obtain an off-white solid. (500 mg, yield: 30%). (H NMR (400 MHz, CDCl₃) δ 8.09 (s, 1H), 5.28 (d, J=54.4 Hz, 1H), 4.22 (dd, J=43.6, 33.5 Hz, 6H), 3.57 (d, J=47.0 Hz, 2H), 3.20 (d, J=26.7 Hz, 3H), 2.98 (s, 1H), 2.37-2.07 (m, 3H), 1.93 (s, 5H), 1.80-1.59 (m, 4H), 1.52 (s, 9H).

### Step 3: Synthesis of tert-butyl (1R,5S)-3-(7-bromo-8-fluoro-2-(((2R,7aS)-2-fluorotetrahydro-1H-pyrrolizin-7a(5H)-yl)methoxy)-6-((triisopropylsilyl)ethynyl)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate (75 mg, 0.104 mmol), Pd(PPh₃)₂Cl₂(2 mg, 0.002 mmol), CuI (1 mg, 0.005 mmol) in diisopropylamine/THF=1/1(3 mL) was added acetylene triisopropylsilane (23 mg, 0.13 mmol). The mixture was stirred under N₂ at 50° C. for 2 h. After the reaction was complete, the mixture was concentrated, purified by prep-TLC (DCM/MeOH=15/1) to give a white solid (60 mg, yield: 74%). (H NMR (400 MHz, CDCl₃) δ 7.71 (s, 1H), 5.28 (d, J=53.3 Hz, 1H), 4.22 (dd, J=44.6, 34.5 Hz, 7H), 3.58 (d, J=84.3 Hz, 3H), 3.23 (s, 4H), 2.37-2.09 (m, 3H), 1.92 (s, 5H), 1.63 (s, 4H), 1.52 (s, 8H), 1.17 (s, 21H).

### Step 4: Synthesis of tert-butyl (1R,5S)-3-(8-fluoro-2-(((2R,7aS)-2-fluorotetrahydro-1H-pyrrolizin-7a(5H)-yl)methoxy)-7-(3-(methoxymethoxy)naphthalen-1-yl)-6-((triisopropylsilyl)ethynyl)quinazolin-4-yl)-3,8-diazabicyclo[3.2.1]octane-8-carboxylate

Compound tert-butyl (1R,5S)-3-(7-bromo-8-fluoro-2-(((2R,7aS)-2-fluorotetrahydro-1H-pyrrolizin-7a(5H)-yl)methoxy)-6-((triisopropylsilyl)ethynyl)quinazolin-4-yl)-3,8-diazabicyclo[3.2.1]octane-8-carboxylate (60 mg, 0.08 mmol), 2-(3-(methoxymethoxy)naphthalen-1-yl)-4,4,5,5-tetramethyl-1,3,2-dioxaborolane (32 mg, 0.10 mmol), Pd(dppf)Cl₂ (12 mg, 0.02 mmol) and K₂CO₃ (22 mg, 0.15 mmol) were stirred in 1,4-dioxane/H₂O (3 mL, 5/1) under N₂ at 100° C. for 4 h. After the reaction was complete, the reaction product was cooled to room temperature, the reaction liquid was diluted by adding water and extracted with ethyl acetate, and the organic phase was dried with anhydrous sodium sulfate, concentrated, separated and purified by prep-TLC (DCM/MeOH=15/1) to obtain an off-white solid. (53 mg, yield: 78%). MS m/z: 882.9 [M+H]⁺

### Step 5: Synthesis of tert-butyl (1R,5S)-3-(6-ethynyl-8-fluoro-2-(((2R,7aS)-2-fluorotetrahydro-1H-pyrrolizin-7a(5H)-yl)methoxy)-7-(3-(methoxymethoxy)naphthalen-1-yV)quinazolin-4-yl)-3,8-diazabicyclo[3.2.1]octane-8-carboxylate

Compound tert-butyl (1R,5S)-3-(8-fluoro-2-(((2R,7aS)-2-fluorotetrahydro-1H-pyrrolizin-7a(5H)-yl)methoxy)-7-(3-(methoxymethoxy)naphthalen-1-yl)-6-((triisopropylsilyl)ethynyl)quinazolin-4-yl)-3,8-diazabicyclo[3.2.1]octane-8-carboxylate (53 mg, 0.06 mmol) and CsF (46 mg, 0.30 mmol) were stirred in DMF (3 mL) at room temperature for 6 h. After the reaction was completed by TLC, the reaction solution was diluted with salt water, extracted with ethyl acetate, the organic phase was dried with anhydrous sodium sulfate, and concentrated to obtain a white-like solid. (43 mg, yield: 100%). MS m/z: 726.7 [M+H]⁺.

### Step 6: Synthesis of 4-(4-((1R,5S)-3,8-diazabicyclo[3.2.1]octan-3-yl)-6-ethynyl-8-fluoro-2-(((2R,7aS)-2-fluorotetrahydro-1H-pyrrolizin-7a(5H)-yl)methoxy)quinazolin-7-yl)naphthalen-2-ol

Compound tert-butyl (1R,5S)-3-(6-ethynyl-8-fluoro-2-(((2R,7aS)-2-fluorotetrahydro-1H-pyrrolizin-7a(5H)-yl)methoxy)-7-(3-(methoxymethoxy)naphthalen-1-yl)quinazolin-4-yl)-3,8-diazabicyclo[3.2.1]octane-8-carboxylate (43 mg, 0.06 mmol) was stirred in CH₃CN (1 mL) and HCl/1,4-dioxane (1 mL, 4N) at room temperature for 30 min. After the reaction was complete, pH of the solution was adjusted to 7-8 with saturated sodium bicarbonate aqueous solution, extracted with ethyl acetate, and the organic phase was dried with anhydrous sodium sulfate, concentrated, purified by prep-TLC (DCM/MeOH(7N NH₃)=15/1) to give a white solid (20 mg, yield: 62.9%). ¹H NMR (400 MHz, CDCl₃) δ 7.82 (d, J=7.4 Hz, 1H), 7.73-7.57 (m, 1H), 7.33 (dt, J=15.3, 7.7 Hz, 2H), 7.24-7.07 (m, 3H), 5.21 (dd, J=54.6, 12.7 Hz, 1H), 4.61-4.06 (m, 4H), 3.80-3.11 (m, 7H), 2.99 (s, 1H), 2.75 (s, 1H), 2.45-2.06 (m, 3H), 2.04-1.82 (m, 3H), 1.75-1.48 (m, 4H). MS m/z: 582.6 [M+H]⁺.

Examples 358-366 were obtained by using the method of Example 357.

| | | | m/z: ES⁺ |
|---|---|---|---|
| Ex. | Compound name | Structural formula | [M + H]⁺ |
| 370 | 4-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)- 5,6,7,8-tetrahydronaphthalen-2-ol | 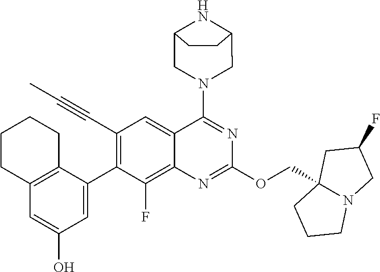 [SMILES: CC#Cc1cc2c(N3CC4CCC(C3)N4)nc(OC[C@@]34CCCN3C[C@H](F)C4)nc2c(F)c1-c1cc(O)cc2c1CCCC2] | 585.6 |
| 371 | 7-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)-1- ethyl-2,3-dihydro-1H-inden-5-ol | 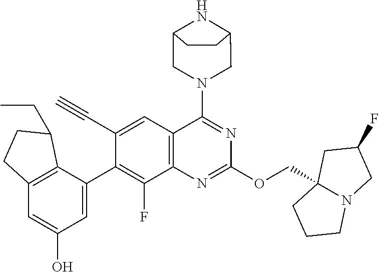 [SMILES: C#Cc1cc2c(N3CC4CCC(C3)N4)nc(OC[C@@]34CCCN3C[C@H](F)C4)nc2c(F)c1-c1cc(O)cc2c1C(CC)CC2] | 600.7 |
| 372 | 5-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)-6- ethyl-1,1a,6,6a-tetrahydrocyclopropa[a]inden-3-ol | 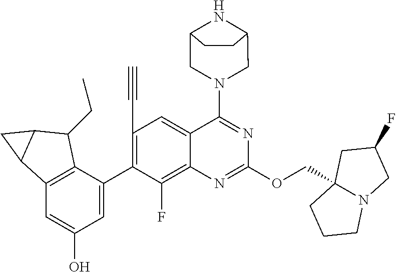 [SMILES: C#Cc1cc2c(N3CC4CCC(C3)N4)nc(OC[C@@]34CCCN3C[C@H](F)C4)nc2c(F)c1-c1cc(O)cc2c1C(CC)C1CC21] | 612.7 |
| 373 | 4-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)-5- ethynyl-6-fluoronaphthalen-2-ol | 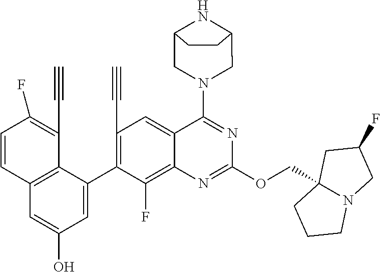 [SMILES: C#Cc1cc2c(N3CC4CCC(C3)N4)nc(OC[C@@]34CCCN3C[C@H](F)C4)nc2c(F)c1-c1cc(O)cc2ccc(F)c(C#C)c12] | 624.6 |
| 374 | 4-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)-5- methyl-5,6,7,8-tetrahydronaphthalen-2-ol | 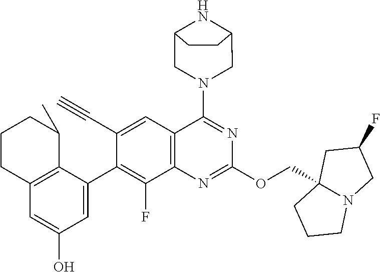 [SMILES: C#Cc1cc2c(N3CC4CCC(C3)N4)nc(OC[C@@]34CCCN3C[C@H](F)C4)nc2c(F)c1-c1cc(O)cc2c1C(C)CCC2] | 600.7 |
| 375 | 8-(4-((1R,5S)-3,8-diazabicyclo[3.2.1]octan-3-yl)-6- ethynyl-8-fluoro-2-(((2R,7aS)-2-fluorotetrahydro-1H- pyrrolizin-7a(5H)-yl)methoxy)quinazolin-7-yl)-1- ethylisoquinolin-6-ol | 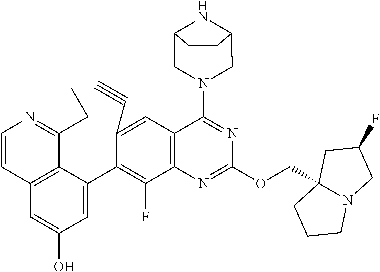 [SMILES: C#Cc1cc2c(N3CC4CCC(C3)N4)nc(OC[C@@]34CCCN3C[C@H](F)C4)nc2c(F)c1-c1cc(O)cc2ccnc(CC)c12] | 611.7 |
| 376 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-6-(1H-pyrazol-4-yl)quinazolin-7- yl)-5-methyl-5,6,7,8-tetrahydronaphthalen-2-ol | 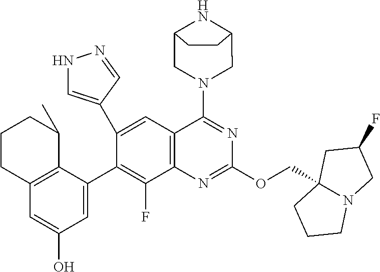 [SMILES: CC1CCCc2cc(O)cc(-c3c(-c4cn[nH]c4)cc4c(N5CC6CCC(C5)N6)nc(OC[C@@]56CCCN5C[C@H](F)C6)nc4c3F)c21] | 642.7 |
| 377 | 4-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)-6-(1H-pyrazol-4-yl)quinazolin-7- yl)naphthalen-2-ol | 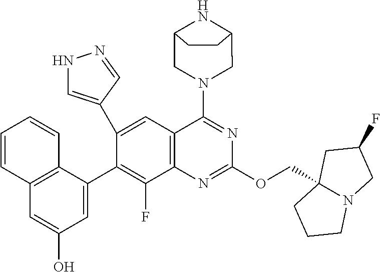 [SMILES: Oc1cc(-c2c(-c3cn[nH]c3)cc3c(N4CC5CCC(C4)N5)nc(OC[C@@]45CCCN4C[C@H](F)C5)nc3c2F)c2ccccc2c1] | 624.7 |
| 378 | 3-(4-((1R,5S)-3,8-diazabicyclo[3.2.1]octan-3-yl)-7-(8- ethynyl-7-fluoro-3-hydroxynaphthalen-1-yl)-8-fluoro- 2-(((2R,7aS)-2-fluorotetrahydro-1H-pyrrolizin- 7a(5H)-yl)methoxy)quinazolin-6-yl)propanenitrile | 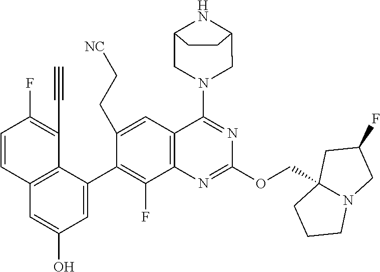 [SMILES: C#Cc1c(F)ccc2cc(O)cc(-c3c(CCC#N)cc4c(N5CC6CCC(C5)N6)nc(OC[C@@]56CCCN5C[C@H](F)C6)nc4c3F)c12] | 653.7 |

### Example 379: Synthesis of 4-(1-((1R,5S)-3,8-diazabicyclo[3.2.1]octan-3-yl)-5-fluoro-3-(((2R,7aS)-2 fluorotetrahydro-1H-pyrrolizin-7a(5H)-yl)methoxy)-8,9-dihydro-7H-cyclopenta[f]quinazolin-6-yl)-5-ethynyl-6 fluoronaphthalen-2-ol

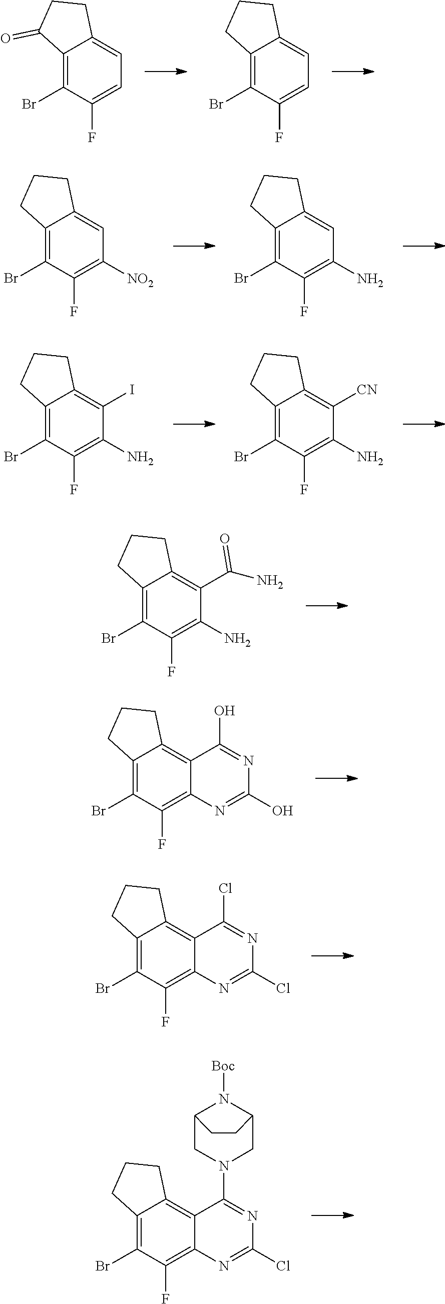

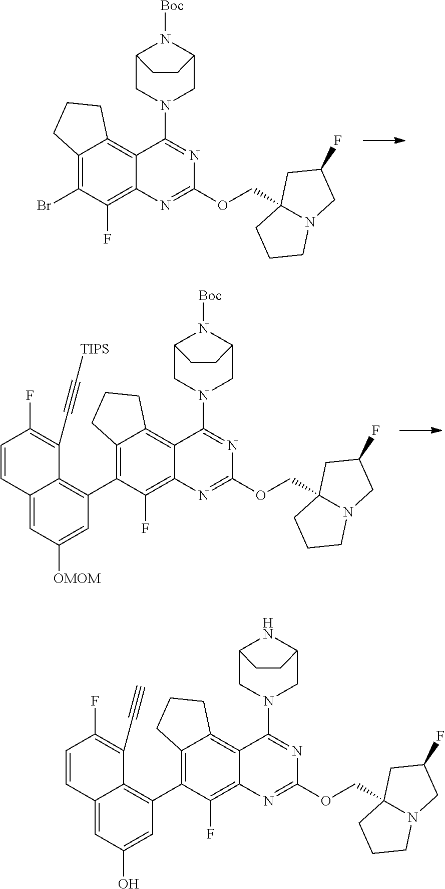

### Step 1: Synthesis of 4-bromo-5-fluoro-2,3-dihydro-1H-indene

Compound 7-bromo-6-fluoro-2,3-dihydro-1H-inden-1-one (4.58 g, 20 mmol) was dissolved in 100 ml TFA, SiHEt₃ (8.4 ml, 50 mmol) was added at room temperature, and the reaction system was stirred overnight. After the reaction was complete, the crude product was obtained by concentration, purified by spectrum (eluent: Petroleum ether/EA=10/1) to obtain a colorless oil (4.1 g, yield: 95%). MS m/z: 215 [M+H]⁺. ¹H NMR (400 MHz, DMSO-D6) δ 7.14 (m, 1H), 6.80 (t, 1H, J=1.3 Hz), 2.85-2.80 (m, 4H), 2.02-1.92 (in, 2H).

### Step 2: Synthesis of 4-bromo-5-fluoro-6-nitro-2,3-dihydro-1H-indene

Compound 4-bromo-5-fluoro-2,3-dihydro-1H-indene (4.1 g, 19 mmol) in concentrated sulfuric acid (40 ml) at 0° C., was added KNO₃ (2.3 g. 22.8 mmol) slowly, and stirred for 30 min. After the reaction was completed, the reaction system was slowly poured onto ice, with a large amount of solid precipitated, extracted with 200 ml EA, and the organic phase was washed with saturated sodium chloride solution. After drying with anhydrous sodium sulfate, it was concentrated to obtain the crude product, purified by column chromatography to obtain a white solid (3.9 g, yield: 79%). MS m/z: 260 [M+H]⁺. MS m/z: 215 [M+H]⁺. ¹H NMR (400 MHz, DMSO-D6) δ 7.61 (m, 1H), 2.85-2.80 (m, 4H), 2.02-1.92 (m, 2H).

### Step 3: Synthesis of 7-bromo-6-fluoro-2,3-dihydro-1H-inden-5-amine

Compound 4-bromo-5-fluoro-6-nitro-2,3-dihydro-1H-indene (3.9 g, 15 mmol) and Fe powder (1.85 g, 33 mmol) were stirred in AcOH (50 ml) under Ar at room temperature overnight. After the reaction was completed, the reaction system was neutralized with a saturated sodium bicarbonate solution, extracted with ethyl acetate, the organic phase was dried with anhydrous sodium sulfate and concentrated to give a light yellow solid (2.3 g, yield: 67%). The light yellow solid was directly used for the next reaction without any further treatment. MS m/z: 230 [M+H]⁺. ¹H NMR (400 MHz, DMSO-D6) δ 6.33 (m, 1H), 5.32 (m, 4H) 2.85-2.80 (m, 4H), 2.02-1.92 (m, 2H).

### Step 4: Synthesis of 7-bromo-6-fluoro-4-iodo-2,3-dihydro-1H-inden-5-amine

Compound 7-bromo-6-fluoro-2,3-dihydro-1H-inden-5-amine (2.3 g, 10 mmol) and NIS (2.7 g, 12 mmol) were stirred in 20 ml DMF at 70° C. overnight. After the reaction was completed, the mixture was concentrated, diluted with 200 ml EA, the organic phase was washed by 100 ml water and 100 ml brine for three times, dried with anhydrous sodium sulfate and concentrated to give a yellow solid (2.1 g, yield: 60%). MS m/z: 355[M+H]⁺.

### Step 5: Synthesis of 5-amino-7-bromo-6-fluoro-2,3-dihydro-1H-indene-4-carbonitrile

Compound 7-bromo-6-fluoro-4-iodo-2,3-dihydro-1H-inden-5-amine (2.1 g, 6 mmol), Zn(CN)₂ (914 mg, 7.8 mmol), 4 Å molecular sieve (100 mg) and Pd(PPh₃)₄(693 mg, 0.6 mmol) were stirred in 10 ml DMF under Ar at 100° C. overnight. After the reaction was completed, the mixture was concentrated, purified by column chromatography to give a yellow solid (581 mg, yield: 38%). MS m/z: 255[M+H]⁺.

### Step 6: Synthesis of 5-amino-7-bromo-6-fluoro-2,3-dihydro-1H-indene-4-carboxamide

Compound 5-amino-7-bromo-6-fluoro-2,3-dihydro-1H-indene-4-carbonitrile (581 mg, 2.28 mmol) and t-BuOK (1.3 g, 11.4 mmol) were stirred in t-BuOH at 70° C. overnight. After the reaction was completed, the mixture was concentrated, extracted with DCM/H₂O, the organic phase was dried with anhydrous sodium sulfate and concentrated to give a white solid (546 mg, yield: 88%). MS m/z: 273[M+H]⁺.

### Step 7: Synthesis of 6-bromo-5-fluoro-8,9-dihydro-7H-cyclopenta[f]quinazoline-1,3-diol

Compound 5-amino-7-bromo-6-fluoro-2,3-dihydro-1H-indene-4-carboxamide (546 mg, 2 mmol), CDI (1.3 g, 8 mmol) and K₂CO₃ (1.1 g, 8 mmol) were stirred in 10 ml DMF at 80° C. overnight. After the reaction was completed, the mixture was diluted with 100 ml Saturated sodium chloride solution, extracted with 500 ml EA, the organic phase was dried with anhydrous sodium sulfate and concentrated to give a white solid, the white solid was directly used for the next reaction without any further treatment.

### Step 8: Synthesis of 6-bromo-1,3-dichloro-5-fluoro-8,9-dihydro-7H-cyclopenta[f]quinazoline

Compound 6-bromo-5-fluoro-8,9-dihydro-7H-cyclopenta[f]quinazoline-1,3-diol (obtained last step) in POCl₃ (10 ml) was added N,N-diethylaniline (1 ml) at room temperature. The resulting suspension was refluxed overnight. After the reaction was complete, the reaction liquid was concentrated and purified by column chromatography (DCM) to obtain a yellow solid. (510 mg, yield: 76%). MS m/z: 336[M+H]⁺.

### Step 9: Synthesis of tert-butyl (1R,5S)-3-(6-bromo-3-chloro-5-fluoro-8,9-dihydro-7H-cyclopenta[f]quinazolin-1-yl)-3,8-diazabicyclo[3.2.1]octane-8-carboxylate

Compound 6-bromo-1,3-dichloro-5-fluoro-8,9-dihydro-7H-cyclopenta[f]quinazoline (510 mg, 1.52 mmol) and tert-butyl (1R,5S)-3,8-diazabicyclo[3.2.1]octane-8-carboxylate (354 mg, 1.67 mmol) in DMF (10 mL) at 0° C. was added DIEA (0.43 mL, 3 mmol). The resulting suspension was stirred at 0° C. for 30 min. After the reaction was complete, the reaction liquid was diluted with water and extracted with ethyl acetate, and the organic phase was washed with a saturated aqueous NaCl solution, dried with anhydrous sodium sulfate, concentrated to obtain a light yellow solid, washed with petroleum ether, filtered and dried in vacuum to obtain a light yellow solid. (715 mg, yield: 92%). MS m/z: 511[M+H]⁺.

### Step 10: Synthesis of tert-butyl (1R,5S)-3-(6-bromo-5-fluoro-3-(((2R,7aS)-2-fluorotetrahydro-1H-pyrrolizin-7a(5H)-yl)methoxy)-8,9-dihydro-7H-cyclopenta[f]quinazolin-1-yl)-3,8-diazabicyclo[3.2.1]octane-8-carboxylate

The compound ((2R,7aS)-2-fluorotetrahydro-1H-pyrrolizin-7a(5H)-yl)methanol (315 mg, 1.98 mmol) was dissolved in anhydrous tetrahydrofuran (10 mL), 60% NaH (73 mg, 1.8 mmol) was added under ice-water bath cooling condition and reacted under stirring for 30 min, and tert-butyl (1R,5S)-3-(6-bromo-3-chloro-5-fluoro-8,9-dihydro-7H-cyclopenta[f]quinazolin-1-yl)-3,8-diazabicyclo[3.2.1]octane-8-carboxylate (510 mg, 1.52 mmol) was added and reacted under stirring overnight. After the reaction was complete, the reaction was quenched with cold water, extracted with ethyl acetate, and the organic phase was washed with a saturated aqueous NaCl solution, dried with anhydrous sodium sulfate, concentrated and separated by column chromatography to obtain an off-white solid. (626 mg, yield: 65%). MS m/z: 634[M+H]⁺.

### Step 11: Synthesis of tert-butyl (1R,5S)-3-(5-fluoro-6-(7-fluoro-3-(methoxymethoxy)-8-((triisopropylsilyl)ethynyl)naphthalen-1-yl)-3-(((2R,7aS)-2-fluorotetrahydro-1H-pyrrolizin-7a(5H)-yl)methoxy)-8,9-dihydro-7H-cyclopenta[f]quinazolin-1-yl)-3,8-diazabicyclo[3.2.1]octane-8-carboxylate

Compound tert-butyl (1R,5S)-3-(6-bromo-5-fluoro-3-(((2R,7aS)-2-fluorotetrahydro-1H-pyrrolizin-7a(5H)-yl)methoxy)-8,9-dihydro-7H-cyclopenta[f]quinazolin-1-yl)-3,8-diazabicyclo[3.2.1]octane-8-carboxylate (63 mg, 0.10 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (70 mg, 0.13 mmol) and Cs₂CO₃ (82 mg, 0.25 mmol) were dissolved in 1,4-dioxane and water (3 ml, 5/1), and after displacement with nitrogen, Pd(dppf)Cl₂ (8 mg, 0.01 mmol) was added. The reaction system was stirred at 100° C. for 12 h. After the reaction was complete, the reaction system was cooled to room temperature, diluted with water, extracted with ethyl acetate and separated, the organic phase was dried with anhydrous sodium sulfate, and the solvent was then removed under reduced pressure to obtain a crude product. The crude product was separated by prep-TLC to obtain a white solid (30 mg, yield: 33%). MS m/z: 940 [M+H]⁺.

### Step 12: Synthesis of 4-(1-((1R,5S)-3,8-diazabicyclo[3.2.1]octan-3-yl)-5-fluoro-3-(((2R,7aS)-2-fluorotetrahydro-1H-pyrrolizin-7a(5H)-yl)methoxy)-8,9-dihydro-7H-cyclopenta[f]quinazolin-6-yl)-5-ethynyl-6-fluoronaphthalen-2-ol

Compound tert-butyl (1R,5S)-3-(5-fluoro-6-(7-fluoro-3-(methoxymethoxy)-8-((triisopropylsilyl)ethynyl)naphthalen-1-yl)-3-(((2R,7aS)-2-fluorotetrahydro-1H-pyrrolizin-7a(5H)-yl)methoxy)-8,9-dihydro-7H-cyclopenta[f]quinazolin-1-yl)-3,8-diazabicyclo[3.2.1]octane-8-carboxylate (30 mg, 0.032 mmol) and CsF (15 mg, 0.095 mmol) in DMF (3 mL) were stirred at room temperature for 4 h. After the reaction was complete by TLC, the reaction was diluted with brine, extracted with ethyl acetate, and the organic phase was dried with anhydrous sodium sulfate, concentrated to give a white solid. Then the crude product was dissolved with CH₃CN (3 mL), HCl/1,4-dioxane (4M, 1 mL) was added and stirred at room temperature for 30 min. After the reaction was complete, pH of the solution was adjusted to 7-8 with saturated sodium bicarbonate aqueous solution, extracted with ethyl acetate, and the organic phase was dried with anhydrous sodium sulfate, concentrated, purified by prep-TLC (DCM/MeOH(7N NH₃)=15/1) to give a yellow solid (18 mg, yield: 88%). MS m/z: 640 [M+H]⁺.

Examples 368-371 were obtained by using the method of Example 367.

| | | | m/z: ES⁺ |
|---|---|---|---|
| Ex. | Compound name | Structural formula | [M + H] |
| 380 | 4-(1-((1R,5S)-3,8-diazabicyclo[3.2.1]octan-3-yl)-5- fluoro-3-(((2R,7aS)-2-fluorotetrahydro-1H- pyrrolizin-7a(5H)-yl)methoxy)-8,9-dihydro-7H- cyclopenta[f]quinazolin-6-yl)-5,6- difluoronaphthalen-2-ol | 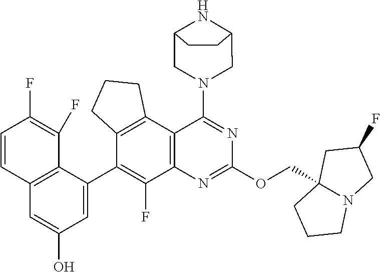 [SMILES: Oc1cc(-c2c3c(c4c(N5CC6CCC(C5)N6)nc(OC[C@@]56CCCN5C[C@H](F)C6)nc4c2F)CCC3)c2c(F)c(F)ccc2c1] | 634 |
| 381 | 7-(1-((1R,5S)-3,8-diazabicyclo[3.2.1]octan-3-yl)-5- fluoro-3-(((2R,7aS)-2-fluorotetrahydro-1H- pyrrolizin-7a(5H)-yl)methoxy)-8,9-dihydro-7H- cyclopenta[f]quinazolin-6-yl)-1-ethyl-2,3-dihydro- 1H-inden-5-ol | 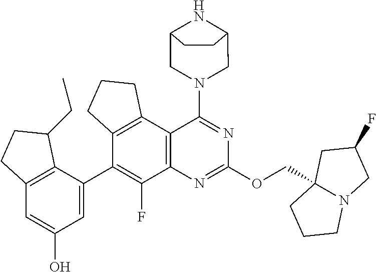 [SMILES: CCC1CCc2cc(O)cc(-c3c4c(c5c(N6CC7CCC(C6)N7)nc(OC[C@@]67CCCN6C[C@H](F)C7)nc5c3F)CCC4)c21] | 616 |
| 382 | 4-(1-((1R,5S)-3,8-diazabicyclo[3.2.1]octan-3-yl)-5- fluoro-3-(((2R,7aS)-2-fluorotetrahydro-1H- pyrrolizin-7a(5H)-yl)methoxy)-8,9-dihydro-7H- cyclopenta[f]quinazolin-6-yl)-5-methyl-5,6,7,8- tetrahydronaphthalen-2-ol | 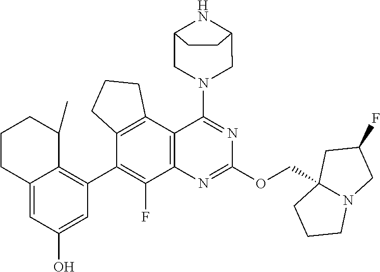 [SMILES: CC1CCCc2cc(O)cc(-c3c4c(c5c(N6CC7CCC(C6)N7)nc(OC[C@@]67CCCN6C[C@H](F)C7)nc5c3F)CCC4)c21] | 616.5 |
| 383 | 4-(1-((1R,5S)-3,8-diazabicyclo[3.2.1]octan-3-yl)-5- fluoro-3-(((2R,7aS)-2-fluorotetrahydro-1H- pyrrolizin-7a(5H)-yl)methoxy)-8,9-dihydro-7H- cyclopenta[f]quinazolin-6-yl)-5-ethyl-5,6,7,8- tetrahydronaphthalen-2-ol | 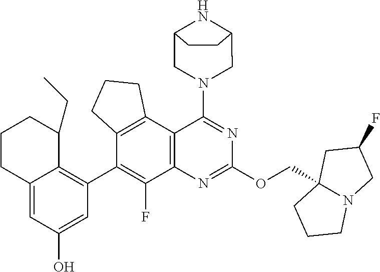 [SMILES: CCC1CCCc2cc(O)cc(-c3c4c(c5c(N6CC7CCC(C6)N7)nc(OC[C@@]67CCCN6C[C@H](F)C7)nc5c3F)CCC4)c21] | 630.5 |

### Example 384: Synthesis of 4-(9-((2R,5S)-3,8-diazabicyclo[3.2.1]octan-3-yl)-7-(((2R,7aS)-2-fluorotetrahydro-1H-pyrrolizin-7a(5H)-yl)methoxy)furo[2,3-f]quinazolin-4-yl)-5-ethynyl-6-fluoronaphthalen-2-ol

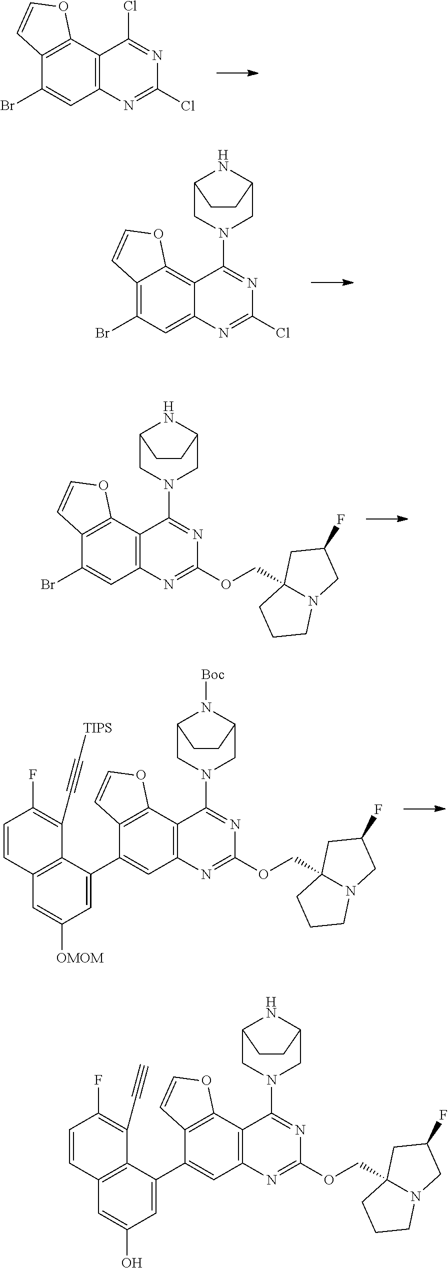

Step 1 to step 4: white solid was obtained by using the synthetic method from steps 9 to 12 of example 367. (1.1 mg, 0.0056 mmol, total yield: 12%). MS m/z: 622.4 [M+H]⁺.

Examples 373-376 were obtained by using the method of Example 372.

| | | | m/z: ES⁺ |
|---|---|---|---|
| Ex. | Compound name | Structural formula | [M + H]⁺ |
| 385 | 7-(9-((1R,5S)-3,8-diazabicyclo[3.2.1]octan-3-yl)-7- (((2R,7aS)-2-fluorotetrahydro-1H-pyrrolizin-7a(5H)- yl)methoxy)furo[2,3-f]quinazolin-4-yl)-1-methyl-2,3- dihydro-1H-inden-5-ol | 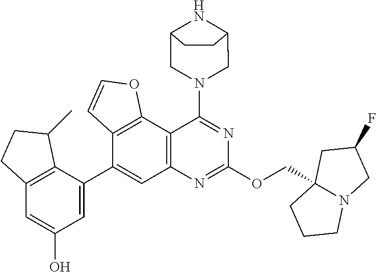 [SMILES: CC1CCc2cc(O)cc(-c3cc4nc(OC[C@@]56CCCN5C[C@H](F)C6)nc(N5CC6CCC(C5)N6)c4c4occc34)c21] | 584.4 |
| 386 | 7-(9-((1R,5S)-3,8-diazabicyclo[3.2.1]octan-3-yl)-7- (((2R,7aS)-2-fluorotetrahydro-1H-pyrrolizin-7a(5H)- yl)methoxy)furo[2,3-f]quinazolin-4-yl)-1-ethyl-2,3- dihydro-1H-inden-5-ol | 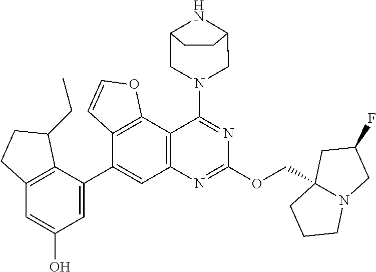 [SMILES: CCC1CCc2cc(O)cc(-c3cc4nc(OC[C@@]56CCCN5C[C@H](F)C6)nc(N5CC6CCC(C5)N6)c4c4occc34)c21] | 597.4 |
| 387 | 4-(9-((1R,5S)-3,8-diazabicyclo[3.2.1]octan-3-yl)-7- (((2R,7aS)-2-fluorotetrahydro-1H-pyrrolizin-7a(5H)- yl)methoxy)furo[2,3-f]quinazolin-4-yl)-5-methyl- 5,6,7,8-tetrahydronaphthalen-2-ol | 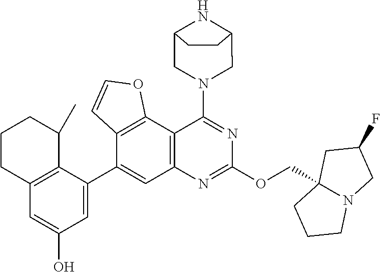 [SMILES: CC1CCCc2cc(O)cc(-c3cc4nc(OC[C@@]56CCCN5C[C@H](F)C6)nc(N5CC6CCC(C5)N6)c4c4occc34)c21] | 598.4 |
| 388 | 4-(9-((1R,5S)-3,8-diazabicyclo[3.2.1]octan-3-yl)-7- (((2R,7aS)-2-fluorotetrahydro-1H-pyrrolizin-7a(5H)- yl)methoxy)furo[2,3-f]quinazolin-4-yl)-5-ethyl-5,6,7,8- tetrahydronaphthalen-2-ol | 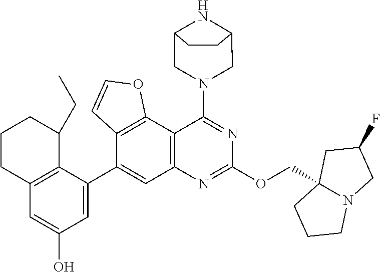 [SMILES: CCC1CCCc2cc(O)cc(-c3cc4nc(OC[C@@]56CCCN5C[C@H](F)C6)nc(N5CC6CCC(C5)N6)c4c4occc34)c21] | 612.4 |

### Example 389: Synthesis of 4-(9-((1R,5S)-3,8-diazabicyclo[3.2.1]octan-3-yl)-5-fluoro-7-(((2R,7aS)-2-fluorotetrahydro-1H-pyrrolizin-7a(5H)-yl)methoxy)-[1,3]dioxolo[4,5-f]quinazolin-4-yl)naphthalen-2-ol

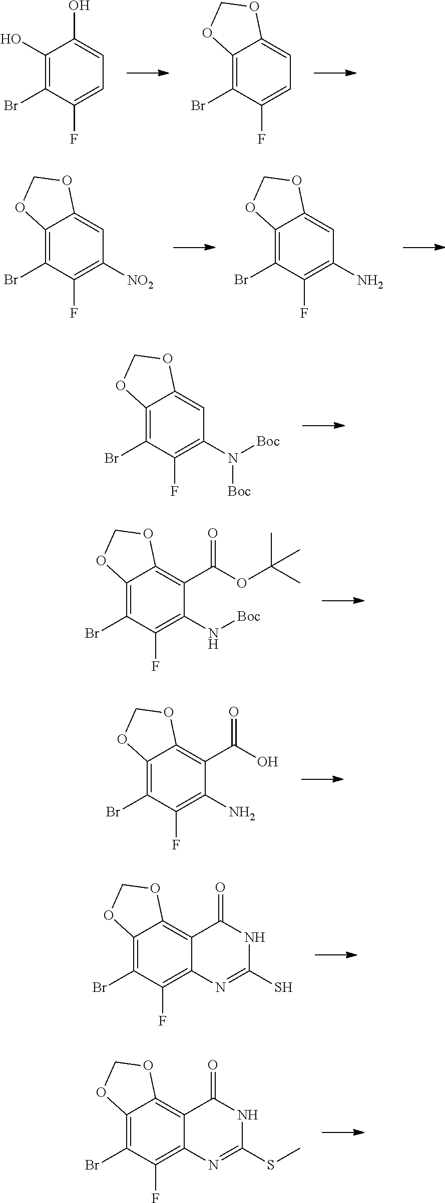

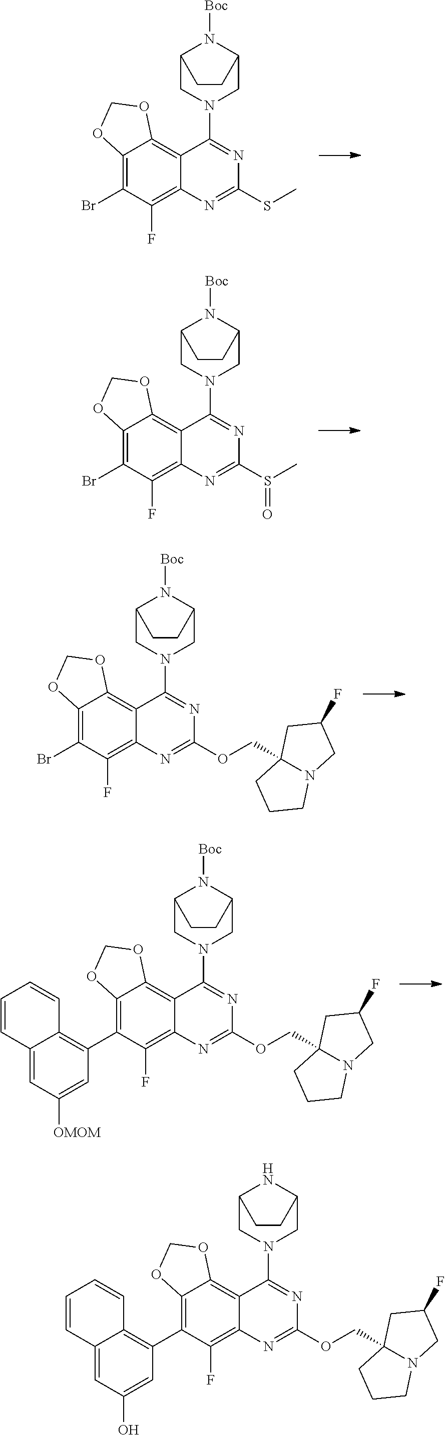

### Step 1: Synthesis of 4-bromo-5-fluorobenzo[d][1,3]dioxole

Compound 3-bromo-4-fluorobenzene-1,2-diol (1.0 g, 4.83 mmol), Cs₂CO₃ (3.96 g, 12.08 mmol) and CH₂I₂(1.42 g, 5.31 mmol) were stirred in DMA (20 mL) at 60° C. for 12 h. After the reaction was completed, cooled to room temperature, the mixture was diluted with water, extracted with ethyl acetate. The organic phase was washed with a saturated aqueous NaCl solution, dried with anhydrous sodium sulfate, concentrated, purified by column chromatography (PE/EA=10/1) to obtain a white solid. (0.82 g, yield: 77.5%).

### Step 2: Synthesis of 4-bromo-5-fluoro-6-nitrobenzo[d][1,3]dioxole

Compound 4-bromo-5-fluorobenzo[d][1,3]dioxole (0.82 g, 3.74 mmol) in AcOH (10 ml) at 0° C., was added KNO₃ (378 mg, 3.74 mmol) slowly, and stirred for 1 h. After the reaction was completed, the reaction system was slowly poured onto ice, extracted with EA, and the organic phase was washed with saturated sodium carbonate. After drying with anhydrous sodium sulfate, it was concentrated to obtain the crude product, purified by column chromatography (PE/EA=5/1) to obtain a yellow solid (0.8 g, yield: 81%).

### Step 3: Synthesis of 7-bromo-6-fluorobenzo[d][1,3]dioxol-5-amine

Compound 4-bromo-5-fluoro-6-nitrobenzo[d][1,3]dioxole (0.8 g, 3.03 mmol) and Fe powder (0.38 g, 6.67 mmol) were stirred in AcOH (50 ml) under Ar at room temperature overnight. After the reaction was completed, the reaction system was neutralized with a saturated sodium bicarbonate solution, extracted with ethyl acetate, the organic phase was dried with anhydrous sodium sulfate and concentrated to give a light yellow solid (0.6 g, yield: 84.6%). The light yellow solid was directly used for the next reaction without any further treatment.

### Step 4: Synthesis of 7-Bromo-6-fluorobenzo[d][1,3]dioxocyclopenten-5-[di (t-butoxycarbonyl)] amine

Compound 7-bromo-6-fluorobenzo[d][1,3]dioxol-5-amine (0.6 g, 2.56 mmol), Boc₂O (1.4 g, 6.41 mmol) and DMAP (16 mg, 0.13 mmol) were stirred in THF (10 mL) at 60° C. for 12 h. After the reaction was completed, the mixture was concentrated, beating with MeOH to give a white solid (1.0 g, yield: 89.8%).

### Step 5: Synthesis of tert-butyl 7-bromo-5-((tert-butoxycarbonyl)amino)-6-fluorobenzo[d][1,3]dioxole-4-carboxylate

Compound 7-Bromo-6-fluorobenzo [d][1,3]dioxocyclopenten-5-[di (t-butoxycarbonyl)] amine (1.0 g, 2.3 mmol) in THF (10 mL) at −70° C., was added to a solution of LDA (2.3 mL, 4.6 mmol, 2M) in THF (20 mL). The mixture was stirred at −70° C. for 2 h. After the reaction was completed, the mixture was quenched by sat. NH₄Cl and extracted with ethyl acetate. The organic phase was washed with sat. NaCl, dried with anhydrous sodium sulfate, concentrated to obtain the crude product, used directly in the next step. (1 g, yield: 100%).

### Step 6: Synthesis of 5-amino-7-bromo-6-fluorobenzo[d][1,3]dioxole-4-carboxylic acid

Compound tert-butyl 7-bromo-5-((tert-butoxycarbonyl)amino)-6-fluorobenzo[d][1,3]dioxole-4-carboxylate (1 g, 2.3 mmol) was stirred in DCM (15 ml) and TFA (5 mL) at room temperature for 3 h. After the reaction was completed, the mixture was concentrated to give the crude product, used directly in the next step.

### Step 7: Synthesis of 4-bromo-5-fluoro-7-mercapto-[1,3]dioxolo[4,5-f]quinazolin-9(8H)-one

Compound 5-amino-7-bromo-6-fluorobenzo[d][1,3]dioxole-4-carboxylic acid (0.64 g, 2.30 mmol) was stirred in SOC₂ (5 mL) at 50° C. for 3 h. After the reaction was completed, the mixture was concentrated, diluted with acetone (10 mL) and it was added to a solution of NH₄SCN (0.53 g, 6.91 mmol) in acetone (10 mL). The mixture was stirred at room temperature for 1 h. After the reaction was completed, the mixture was diluted with water, extracted with ethyl acetate. The organic phase was washed with sat. NaCl, dried with anhydrous sodium sulfate, concentrated to give the crude product, used directly in the next step. (0.5 g, yield: 68%).

### Step 8: Synthesis of 4-bromo-5-fluoro-7-(methylthio)-[1,3]dioxolo[4,5-f]quinazolin-9(8H)-one

Compound 4-bromo-5-fluoro-7-mercapto-[1,3]dioxolo[4,5-f]quinazolin-9(8H)-one (0.5 g, 1.57 mmol), MeONa/MeOH (1 mL, 40%) and Mel (245 mg, 1.72 mmol) were stirred in MeOH (10 mL) at room temperature for 1 h. After the reaction was completed, the mixture was concentrated, diluted with water, extracted with ethyl acetate. The organic phase was washed with sat. NaCl, dried with anhydrous sodium sulfate, concentrated, purified by column chromatography to give a yellow solid (0.5 g, yield: 95%).

### Step 9: Synthesis of tert-butyl (1R,5S)-3-(4-bromo-5-fluoro-7-(methylthio)-[1,3]dioxolo[4,5-f]quinazolin-9-yl)-3,8-diazabicyclo[3.2.1]octane-8-carboxylate

Compound 4-bromo-5-fluoro-7-(methylthio)-[1,3]dioxolo[4,5-f]quinazolin-9(8H)-one (500 mg, 1.5 mmol), DIEA (291 mg, 2.25 mmol) and POCl₃ (277 mg, 1.8 mmol) were stirred in CH₃CN (10 mL) at 80° C. for 1.5 h. After the reaction was completed, the mixture was cooled to 0-10° C., DIEA (291 mg, 2.25 mmol) and tert-butyl (1R,5S)-3,8-diazabicyclo[3.2.1]octane-8-carboxylate (319 mg, 1.5 mmol) were added and stirred at room temperature for 30 min. After the reaction was completed, the mixture was diluted with water, extracted with ethyl acetate. The organic phase was washed with sat. NaCl, dried with anhydrous sodium sulfate, concentrated to give a yellow solid, beating with PE to give an off-white solid. (720 mg, yield: 91%). MS m/z: 527.4 [M+H]⁺

### Step 10: Synthesis of tert-butyl (1R,5S)-3-(4-bromo-5-fluoro-7-(methylsulfinyl)-[1,3]dioxolo[4,5-f]quinazolin-9-yl)-3,8-diazabicyclo[3.2.1]octane-8-carboxylate

Compound tert-butyl (1R,5S)-3-(4-bromo-5-fluoro-7-(methylthio)-[1,3]dioxolo[4,5-f]quinazolin-9-yl)-3,8-diazabicyclo[3.2.1]octane-8-carboxylate (700 mg, 1.33 mmol) and m-CPBA (275 mg, 1.59 mmol, 85%) were stirred in DCM (10 mL) under ice bath cooling for 30 min. After the reaction was complete, the reaction was quenched by using a saturated sodium bisulfite solution and extracted with dichloromethane, and the organic phase was washed with saturated sodium bicarbonate and brine, dried with anhydrous sodium sulfate and concentrated to obtain an off-white solid, which was directly used for the next step. (721 mg, yield: 100%).

### Step 11: Synthesis of tert-butyl (1R,5S)-3-(4-bromo-5-fluoro-7-(((2R,7aS)-2-fluorotetrahydro-1H-pyrrolizin-7a(5H)-yl)methoxy)-[1,3]dioxolo[4,5-f]quinazolin-9-yl)-3,8-diazabicyclo[3.2.1]octane-8-carboxylate

The compound ((2R,7aS)-2-fluorotetrahydro-1H-pyrrolizin-7a(5H)-yl)methanol (275 mg, 1.32 mmol) was dissolved in anhydrous tetrahydrofuran (5 mL), 60% NaH (39 mg, 1.59 mmol) was added under ice-water bath cooling condition and reacted at room temperature under stirring for 20 min, and tert-butyl (1R,5S)-3-(4-bromo-5-fluoro-7-(methylsulfinyl)-[1,3]dioxolo[4,5-f]quinazolin-9-yl)-3,8-diazabicyclo[3.2.1]octane-8-carboxylate (720 mg, 1.72 mmol) was added and reacted at room temperature under stirring for 8 h. After the reaction was complete, the reaction was quenched with cold water, extracted with ethyl acetate, and the organic phase was washed with a saturated aqueous NaCl solution, dried with anhydrous sodium sulfate, concentrated and separated by column chromatography to obtain an off-white solid. (450 mg, yield: 53.1%). MS m/z: 838.4 [M+H]⁺.

### Step 12: Synthesis of tert-butyl (1R,5S)-3-(5-fluoro-7-(((2R,7aS)-2-fluorotetrahydro-1H-pyrrolizin-7a(5H)-yl)methoxy)-4-(3-(methoxymethoxy)naphthalen-1-yl)-[1,3]dioxolo[4,5-f]quinazolin-9-yl)-3,8-diazabicyclo[3.2.1]octane-8-carboxylate

Compound tert-butyl (1R,5S)-3-(4-bromo-5-fluoro-7-(((2R,7aS)-2-fluorotetrahydro-1H-pyrrolizin-7a(5H)-yl)methoxy)-[1,3]dioxolo[4,5-f]quinazolin-9-yl)-3,8-diazabicyclo[3.2.1]octane-8-carboxylate (30 mg, 0.047 mmol), 2-(3-(methoxymethoxy)naphthalen-1-yl)-4,4,5,5-tetramethyl-1,3,2-dioxaborolane (18 mg, 0.056 mmol), Cs₂CO₃ (31 mg, 0.094 mmol) and Pd(PPh₃)₄ (27 mg, 0.023 mmol) were stirred in 1,4-dioxane/H₂O (5 ml/1 ml) under Ar at 100° C. for 4 h. After the reaction was complete, the mixture was concentrated to give the crude product, purified by prep-TLC to give an off-white solid. (25 mg, yield: 71.3%). MS m/z: 746.8 [M+H]⁺.

### Step 13: Synthesis of 4-(9-((1R,5S)-3,8-diazabicyclo[3.2.1]octan-3-yl)-5-fluoro-7-(((2R,7aS)-2-fluorotetrahydro-1H-pyrrolizin-7a(5H)-yl)methoxy)-[1,3]dioxolo[4,5-f]quinazolin-4-yl)naphthalen-2-ol

Compound tert-butyl (1R,5S)-3-(5-fluoro-7-(((2R,7aS)-2-fluorotetrahydro-1H-pyrrolizin-7a(5H)-yl)methoxy)-4-(3-(methoxymethoxy)naphthalen-1-yl)-[1,3]dioxolo[4,5-f]quinazolin-9-yl)-3,8-diazabicyclo[3.2.1]octane-8-carboxylate (25 mg, 0.034 mmol) was stirred in CH₃CN (2 mL) and HCl/1,4-dioxane (1 mL, 4N) at room temperature for 30 min. After the reaction was complete, pH of the solution was adjusted to 7-8 with saturated sodium bicarbonate aqueous solution, extracted with ethyl acetate, and the organic phase was dried with anhydrous sodium sulfate, concentrated, purified by prep-TLC (DCM/MeOH (7N NH₃)=15/1) to give a white solid (14 mg, yield: 69.4%). MS m/z: 602.6 [M+H]⁺

Examples 378-385 were obtained by using the method of Example 377.

| | | | m/z: ES⁺ |
|---|---|---|---|
| Ex. | Compound name | Structural formula | [M + H]⁺ |
| 390 | 4-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-5,6,7,8-tetrahydronaphthalen-2-ol | 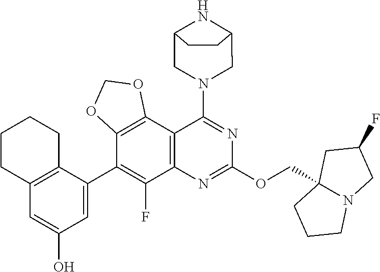 [SMILES: Oc1cc2c(c(-c3c4c(c5c(N6CC7CCC(C6)N7)nc(OC[C@@]67CCCN6C[C@H](F)C7)nc5c3F)OCO4)c1)CCCC2] | 606.6 |
| 391 | 4-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-5-methyl-5,6,7,8-tetrahydronaphthalen-2-ol | 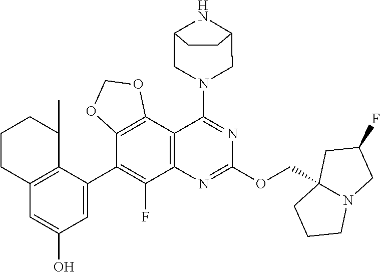 [SMILES: CC1CCCc2cc(O)cc(-c3c4c(c5c(N6CC7CCC(C6)N7)nc(OC[C@@]67CCCN6C[C@H](F)C7)nc5c3F)OCO4)c21] | 620.7 |
| 392 | 8-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-1-methylisochroman-6-ol | 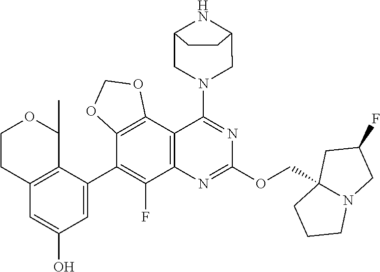 [SMILES: CC1OCCc2cc(O)cc(-c3c4c(c5c(N6CC7CCC(C6)N7)nc(OC[C@@]67CCCN6C[C@H](F)C7)nc5c3F)OCO4)c21] | 622.7 |
| 393 | 4-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-5-ethynyl-6-fluoronaphthalen-2-ol | 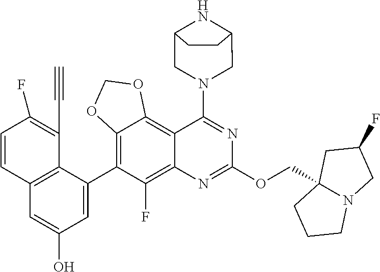 [SMILES: C#Cc1c(F)ccc2cc(O)cc(-c3c4c(c5c(N6CC7CCC(C6)N7)nc(OC[C@@]67CCCN6C[C@H](F)C7)nc5c3F)OCO4)c12] | 644.6 |
| 394 | 7-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-1-ethyl-2,3-dihydro-1H-inden-5-ol | 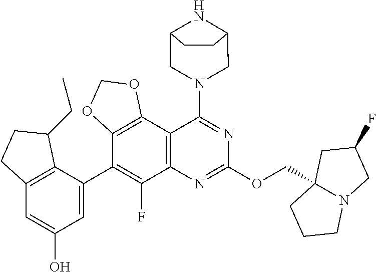 [SMILES: CCC1CCc2cc(O)cc(-c3c4c(c5c(N6CC7CCC(C6)N7)nc(OC[C@@]67CCCN6C[C@H](F)C7)nc5c3F)OCO4)c21] | 620.7 |
| 395 | 8-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-1-ethylisoquinolin-6-ol | 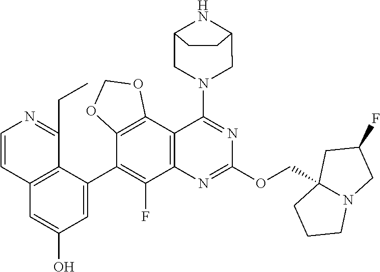 [SMILES: CCc1nccc2cc(O)cc(-c3c4c(c5c(N6CC7CCC(C6)N7)nc(OC[C@@]67CCCN6C[C@H](F)C7)nc5c3F)OCO4)c12] | 631.7 |
| 396 | 5-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-3-ethylimidazo[1,5-a]pyridin-7-ol | 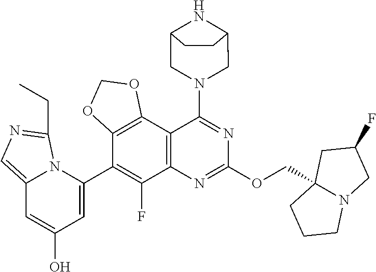 [SMILES: CCc1ncc2cc(O)cc(-c3c4c(c5c(N6CC7CCC(C6)N7)nc(OC[C@@]67CCCN6C[C@H](F)C7)nc5c3F)OCO4)n12] | 620.6 |
| 397 | 5-(9-((1R,5S)-3,8-diazabicyclo[3.2.1]octan-3-yl)-5- fluoro-7-(((2R,7aS)-2-fluorotetrahydro-1H-pyrrolizin- 7a(5H)-yl)methoxy)-[1,3]dioxolo[4,5-f]quinazolin-4- yl)-3-ethylindolizin-7-ol | 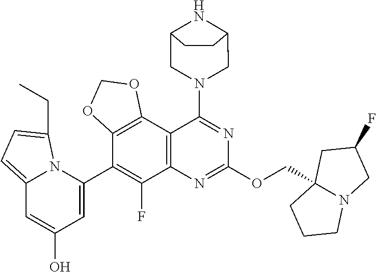 [SMILES: CCc1ccc2cc(O)cc(-c3c4c(c5c(N6CC7CCC(C6)N7)nc(OC[C@@]67CCCN6C[C@H](F)C7)nc5c3F)OCO4)n12] | 619.6 |

### Example 398: Synthesis of 4-C₁-((1R,5S)-3,8-diazabicyclo[3.2.1]octan-3-yl)-5-fluoro-3-(((2R,7aS)-2-fluorotetrahydro-1H-pyrrolizin-7a(5H)-yl)methoxy)imidazo[1′,5′:1,2]pyrido[4,3-d]pyrimidin-6-yl)-5-ethynyl-6-fluoronaphthalen-2-ol

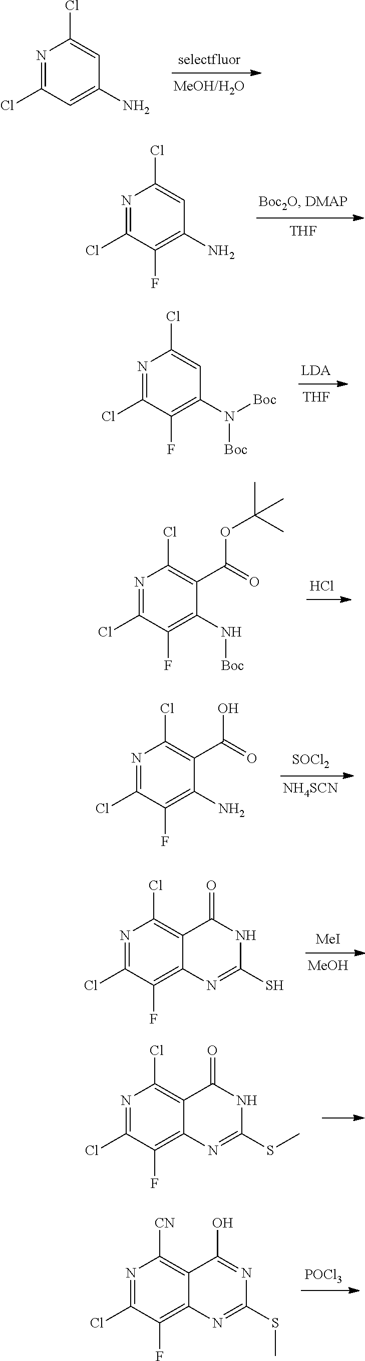

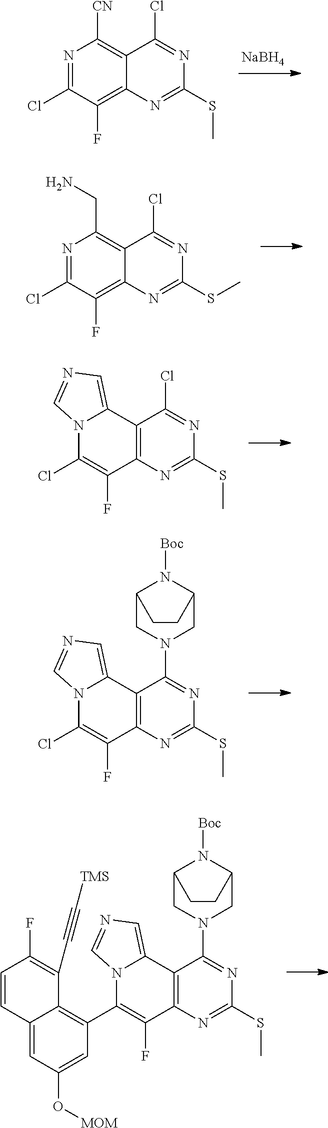

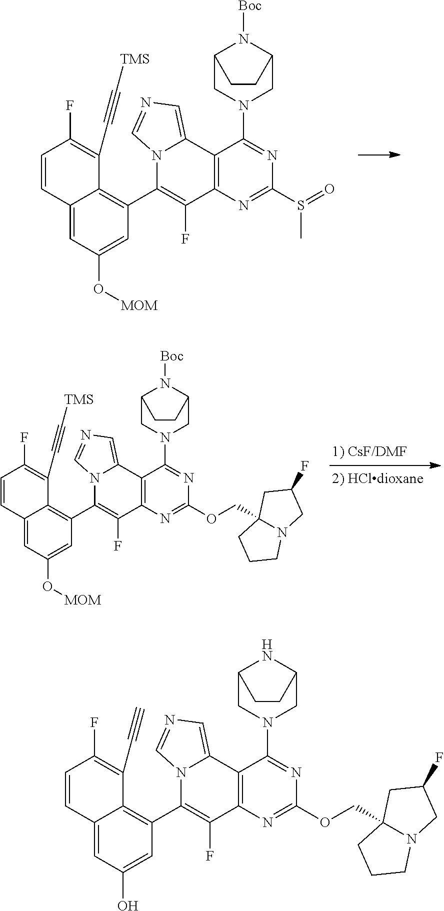

### Step 1: Synthesis of 2,6-dichloro-3-fluoropyridin-4-amine

Compound 2,6-dichloropyridin-4-amine (5 g, 30.5 mmol) and selectfluor (11.3 g, 33.5 mmol) were stirred in MeOH/1H₂O (50 mL/10 mL) at 50° C. for 24 h. After the reaction was complete, the reaction liquid was diluted with water and extracted with ethyl acetate, and the organic phase was washed with brine, dried with anhydrous sodium sulfate, concentrated, and purified by column chromatography to obtain a white solid. (2.5 g, yield: 45%).

### Step 2: Synthesis of 3-fluoro-4-[di (t-butoxycarbonyl) amino]-2,6-dichloropyridine

Compound 2,6-dichloro-3-fluoropyridin-4-amine (2.5 g, 13.8 mmol), BOC₂O (7.53 g, 34.5 mmol) and DMAP (84 mg, 0.7 mmol) were stirred in THF (25 mL) at 60° C. for 12 h. After the reaction was complete, the reaction liquid was concentrated, beating with MeOH to give a white solid. (5.1 g, yield: 97%).

### Step 3: Synthesis of tert-butyl 4-((tert-butoxycarbonyl)amino)-2,6-dichloro-5-fluoronicotinate

Compound 3-fluoro-4-[di (t-butoxycarbonyl) amino]-2,6-dichloropyridine (5.1 g, 13.6 mmol) in THF (10 mL) at −70° C., was added to a solution of LDA (20.5 mL, 40.9 mmol, 2M) in THF (80 mL). The mixture was stirred at −70° C. for 2 h. After the reaction was completed, the mixture was quenched by sat. NH₄Cl and extracted with ethyl acetate. The organic phase was washed with sat. NaCl, dried with anhydrous sodium sulfate, concentrated to obtain the crude product, used directly in the next step. (5.2 g, yield: 100%).

### Step 4: Synthesis of 4-amino-2,6-dichloro-5-fluoronicotinic acid

Compound tert-butyl 4-((tert-butoxycarbonyl)amino)-2,6-dichloro-5-fluoronicotinate (5.2 g, 13.6 mmol) was stirred in concentrated hydrochloric acid (13 ml) and 1,4-dioxane (42 mL) at room temperature for 18 h. After the reaction was completed, the mixture was concentrated to give the crude product, used directly in the next step. (3 g, yield: 100%).

### Step 5: Synthesis of 5,7-dichloro-8-fluoro-2-mercaptopyrido[4,3-d]pyrimidin-4(3H)-one

Compound 4-amino-2,6-dichloro-5-fluoronicotinic acid (3 g, 13.6 mmol) was stirred in SOC₂ (30 mL) at 50° C. for 3 h. After the reaction was completed, the mixture was concentrated, diluted with acetone (10 mL) and it was added to a solution of NH₄SCN (3.1 g, 40.8 mmol) in acetone (40 mL). The mixture was stirred at room temperature for 1 h. After the reaction was completed, the mixture was diluted with water, extracted with ethyl acetate. The organic phase was washed with sat. NaCl, dried with anhydrous sodium sulfate, concentrated to give the crude product, used directly in the next step. (3.6 g, yield: 100%).

### Step 6: Synthesis of 5,7-dichloro-8-fluoro-2-(methylthio)pyrido[4,3-d]pyrimidin-4(3H)-one

Compound 5,7-dichloro-8-fluoro-2-mercaptopyrido[4,3-d]pyrimidin-4(3H)-one (3.6 g, 13.6 mmol), NaOH (272 mL, 27.2 mmol, 0.1M) and Mel (3.9 g, 27.2 mmol) were stirred in MeOH (272 mL) at room temperature for 1 h. After the reaction was completed, the mixture was concentrated, diluted with water, extracted with ethyl acetate. The organic phase was washed with sat. NaCl, dried with anhydrous sodium sulfate, concentrated, purified by column chromatography to give a yellow solid (1.7 g, yield: 45%).

### Step 7: Synthesis of 7-chloro-8-fluoro-4-hydroxy-2-(methylthio)pyrido[4,3-d]pyrimidine-5-carbonitrile

Compound 5,7-dichloro-8-fluoro-2-(methylthio)pyrido[4,3-d]pyrimidin-4(3H)-one (1 g, 3.6 mmol), Zn(CN)₂ (626 mg, 5.4 mmol), DPPF (199 mg, 0.36 mmol) and Pd₂dba₃ (330 mg, 0.36 mmol) were stirred in 5 ml DMF under N₂ at 90° C. for 8 h. After the reaction was completed, the mixture was cooled to room temperature, diluted with water, extracted with ethyl acetate. The organic phase was washed with sat. NaCl, dried with anhydrous sodium sulfate, concentrated, purified by column chromatography to give a yellow solid (750 mg, yield: 78%).

### Step 8: Synthesis of 4,7-dichloro-8-fluoro-2-(methylthio)pyrido[4,3-d]pyrimidine-5-carbonitrile

Compound 7-chloro-8-fluoro-4-hydroxy-2-(methylthio)pyrido[4,3-d]pyrimidine-5-carbonitrile (750 mg, 2.8 mmol) in POCl₃ (9 ml) was added N,N-diethylaniline (1 ml) at room temperature. The resulting suspension was stirred at 110° C. overnight. After the reaction was complete, the reaction liquid was concentrated to obtain the crude product, used directly in the next step. (800 mg, yield: 100%).

### Step 9: Synthesis of (4,7-dichloro-8-fluoro-2-(methylthio)pyrido[4,3-d]pyrimidin-5-yl)methanamine

Compound 4,7-dichloro-8-fluoro-2-(methylthio)pyrido[4,3-d]pyrimidine-5-carbonitrile (800 mg, 2.8 mmol) and NaBH₄ (106 mg, 2.8 mmol) were stirred in THF (10 mL) at room temperature for 5 h. After the reaction was complete, the reaction was quenched with water, extracted with ethyl acetate, and the organic phase was washed with a saturated aqueous NaCl solution, dried with anhydrous sodium sulfate, concentrated to obtain the crude product, used directly in the next step. (810 mg, yield: 100%).

### Step 10: Synthesis of 1,6-dichloro-5-fluoro-3-(methylthio)imidazo[1′,5′:1,21pyrido[4,3-d]pyrimidine

Compound (4,7-dichloro-8-fluoro-2-(methylthio)pyrido[4,3-d]pyrimidin-5-yl)methanamine (810 mg, 2.8 mmol) was stirred in anhydrous ethyl formate (10 mL) at 65° C. for 3 h. After the reaction was complete, the mixture was cooled to room temperature, concentrated, diluted with DCM, then POCl₃ (638 mg, 4.2 mmol) and TEA (848 mg, 8.4 mmol) were added and stirred at room temperature for 2 h. After the reaction was complete, the reaction liquid was diluted with water and extracted with DCM, and the organic phase was dried with anhydrous sodium sulfate, concentrated and separated by column chromatography to obtain a light yellow solid. (470 mg, yield: 56%).

### Step 11: Synthesis of tert-butyl (1R,5S)-3-(6-chloro-5-fluoro-3-(methylthio)imidazo[1′,5′:1,2]pyrido[4,3-d]pyrimidin-1-yl)-3,8-diazabicyclo[3.2.1]octane-8-carboxylate

Compound 1,6-dichloro-5-fluoro-3-(methylthio)imidazo[1′,5′:1,2]pyrido[4,3-d]pyrimidine (470 mg, 1.6 mmol) and tert-butyl (1R,5S)-3,8-diazabicyclo[3.2.1]octane-8-carboxylate (360 mg, 1.7 mmol) in anhydrous DMF (8 mL) under ice bath cooling, was added DIEA (310 mg, 2.4 mmol) and stirred for 30 min. After the reaction was complete, the reaction liquid was diluted with water and extracted with ethyl acetate, and the organic phase was washed with a saturated aqueous NaCl solution, dried with anhydrous sodium sulfate, concentrated to obtain a light yellow solid, washed with PE, filtered, dried to give a light yellow solid (510 mg, yield: 69%).

### Step 12: Synthesis of tert-butyl (1R,5S)-3-(5-fluoro-6-(7-fluoro-3-(methoxymethoxy)-8-((trimethylsilyl)ethynyl)naphthalen-1-yl)-3-(methylthio)imidazo[1′,5′:1,2]pyrido[4,3-d]pyrimidin-1-yl)-3,8-diazabicyclo[3.2.1]octane-8-carboxylate

Compound tert-butyl (1R,5S)-3-(6-chloro-5-fluoro-3-(methylthio)imidazo[1′,5′:1,2]pyrido[4,3-d]pyrimidin-1-yl)-3,8-diazabicyclo[3.2.1]octane-8-carboxylate (48 mg, 0.1 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (57 mg, 0.11 mmol) and Cs₂CO₃ (74 mg, 0.23 mmol) were dissolved in 1,4-dioxane and water (3 ml, 5/1), and after displacement with nitrogen, Pd(dppf)Cl₂ (13 mg, 0.02 mmol) was added. The reaction system was stirred at 100° C. for 5 h. After the reaction was complete, the reaction system was cooled to room temperature, diluted with water, extracted with ethyl acetate and separated, the organic phase was dried with anhydrous sodium sulfate, and the solvent was then removed under reduced pressure to obtain a crude product. The crude product was separated by prep-TLC to obtain an off-white solid (32 mg, yield: 43%).

### Step 13: Synthesis of tert-butyl (1R,5S)-3-(5-fluoro-6-(7-fluoro-3-(methoxymethoxy)-8-((trimethylsilyl)ethynyl)naphthalen-1-yl)-3-(methylsulfinyl)imidazo[1′,5′:1,2pyrido[4,3-d]pyrimidin-1-yl)-3,8-diazabicyclo[3.2.1]octane-8-carboxylate

Compound tert-butyl (1R,5S)-3-(5-fluoro-6-(7-fluoro-3-(methoxymethoxy)-8-((trimethylsilyl)ethynyl)naphthalen-1-yl)-3-(methylthio)imidazo[1′,5′:1,2]pyrido[4,3-d]pyrimidin-1-yl)-3,8-diazabicyclo[3.2.1]octane-8-carboxylate (32 mg, 0.043 mmol) and 85% m-CPBA (11 mg, 0.052 mmol) were stirred in DCM (2 mL) at 0° C.-10° C. for 20 min. After the reaction was complete, the reaction was quenched by saturated sodium thiosulfate solution and extracted with dichloromethane, and the organic phase was washed with saturated sodium bicarbonate and brine, dried with anhydrous sodium sulfate and concentrated to obtain a yellow solid, which was directly used for the next step. (32 mg, yield: 100%).

### Step 14: Synthesis of tert-butyl (1R,5S)-3-(5-fluoro-6-(7-fluoro-3-(methoxymethoxy)-8-((trimethylsilyl)ethynyl)naphthalen-1-yl)-3-(((2R,7aS)-2-fluorotetrahydro-1H-pyrrolizin-7a(5H)-yl)methoxy)imidazo[1′,5′:1,2]pyrido[4,3-d]pyrimidin-1-yl)-3,8-diazabicyclo[3.2.1]octane-8-carboxylate

Compound tert-butyl (1R,5S)-3-(5-fluoro-6-(7-fluoro-3-(methoxymethoxy)-8-((trimethylsilyl)ethynyl)naphthalen-1-yl)-3-(methylsulfinyl)imidazo[1′,5′:1,2]pyrido[4,3-d]pyrimidin-1-yl)-3,8-diazabicyclo[3.2.1]octane-8-carboxylate (32 mg, 0.042 mmol) and ((2R,7aS)-2-fluorotetrahydro-1H-pyrrolizin-7a(5H)-yl)methanol (7 mg, 0.046 mmol) in anhydrous toluene (2 mL) under ice bath cooling, was added t-BuNa (5 mg, 0.05 mmol). The mixture was stirred for 30 min under ice bath cooling. After the reaction was complete, the reaction was quenched with cold water and extracted with EA, and the organic phase was washed with a saturated aqueous NaCl solution, dried with anhydrous sodium sulfate, concentrated and separated by prep-TLC to obtain an off-white solid. (25 mg, yield: 69%).

### Step 15: Synthesis of 4-(1-((1R,5S)-3,8-diazabicyclo[3.2.1]octan-3-yl)-5-fluoro-3-(((2R,7aS)-2-fluorotetrahydro-1H-pyrrolizin-7a(5H)-yl)methoxy)imidazo[1′,5′:1,2]pyrido[4,3-d]pyrimidin-6-yl)-5-ethynyl-6-fluoronaphthalen-2-ol

Compound tert-butyl (1R,5S)-3-(5-fluoro-6-(7-fluoro-3-(methoxymethoxy)-8-((trimethylsilyl)ethynyl)naphthalen-1-yl)-3-(((2R,7aS)-2-fluorotetrahydro-1H-pyrrolizin-7a(5H)-yl)methoxy)imidazo[1′,5′:1,2]pyrido[4,3-d]pyrimidin-1-yl)-3,8-diazabicyclo[3.2.1]octane-8-carboxylate (25 mg, 0.03 mmol) and CsF (30 mg, 0.20 mmol) in DMF (1 mL) were stirred at room temperature for 2 h. After the reaction was complete, the reaction was diluted with water, extracted with ethyl acetate, and the organic phase was washed with a saturated aqueous NaCl solution, dried with anhydrous sodium sulfate, concentrated to give the crude product. Then the crude product was dissolved with CH₃CN (0.5 mL), HCl/1,4-dioxane (4M, 0.5 mL) was added and stirred at room temperature for 30 min. After the reaction was complete, pH of the solution was adjusted to 7 with saturated sodium bicarbonate aqueous solution, extracted with DCM, and the organic phase was dried with anhydrous sodium sulfate, concentrated, purified by prep-TLC (DCM/MeOH(7N NH₃)=10/1) to give a yellow solid (13 mg, yield: 70%). ¹H NMR (400 MHz, Chloroform) δ 8.69 (d, J=2.9 Hz, 1H), 8.26 (s, 1H), 7.85 (d, J=0.6 Hz, 1H), 7.68 (ddd, J=15.0, 10.0, 3.0 Hz, 1H), 7.57 (t, J=3.0 Hz, 1H), 6.89 (dd, J=15.9, 15.0 Hz, 1H), 5.17-4.87 (m, 2H), 4.87-4.77 (m, 2H), 4.49 (q, J=24.8 Hz, 2H), 3.84-3.75 (m, 2H), 3.59 (ddd, J=50.4, 24.7, 14.9 Hz, 1H), 3.37-3.18 (m, 4H), 2.65-2.18 (m, 3H), 2.15-2.00 (m, 2H), 1.76-1.63 (m, 2H), 1.51 (ddd, J=26.0, 13.9, 12.0 Hz, 1H), 1.43-1.19 (m, 4H), 1.06 (s, 1H).

### Example 399: Synthesis of 4-(10-((1R,5S)-3,8-diazabicyclo[3.2.1]octan-3-yl)-6-fluoro-8-(((2R,7aS)-2-fluorotetrahydro-1H-pyrrolizin-7a(5H)-yl)methoxy)imidazo[1′,2′:1,2]pyrido[4,3-d]pyrimidin-5-yl)-5-ethynyl-6-fluoronaphthalen-2-ol

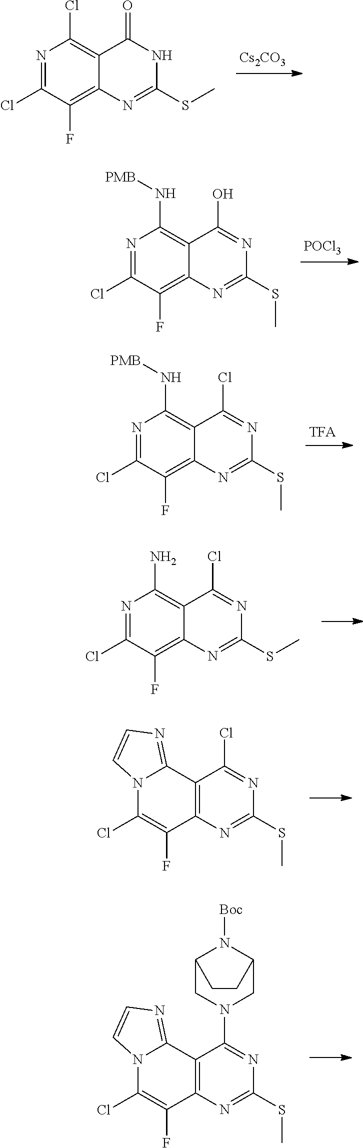

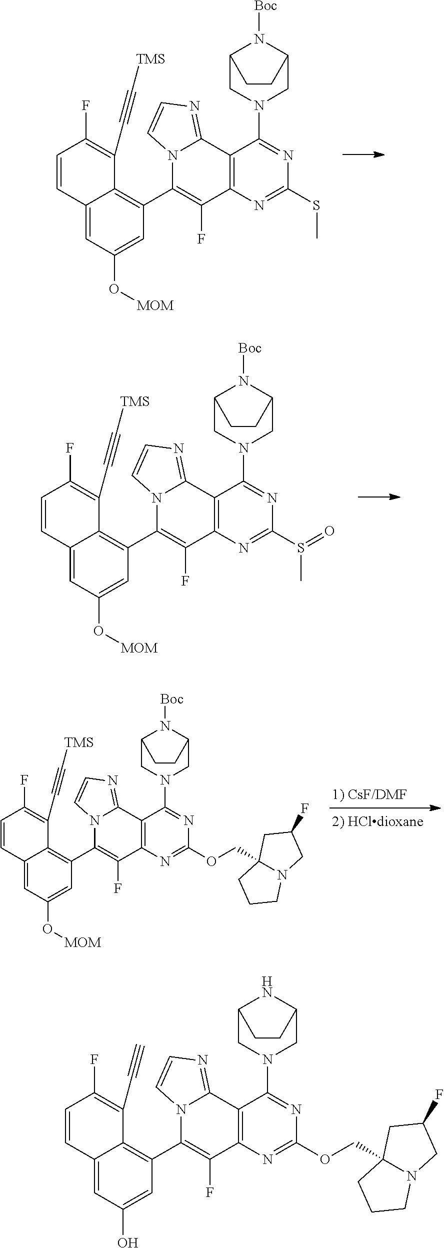

### Step 1: Synthesis of 7-chloro-8-fluoro-5-((4-methoxybenzyl)amino)-2-(methylthio)pyrido[4,3-d]pyrimidin-4-ol

Compound 5,7-dichloro-8-fluoro-2-(methylthio)pyrido[4,3-d]pyrimidin-4(3H)-one (1 g, 3.6 mmol), PMBNH₂ (1.5 g, 10.7 mmol) and Cs₂CO₃ (3.5 g, 10.7 mmol) were stirred in DMA (5 mL) at 100° C. for 24 h. After the reaction was complete, the reaction liquid was cooled to room temperature, diluted with water and extracted with ethyl acetate, and the organic phase was washed with a saturated aqueous NaCl solution, dried with anhydrous sodium sulfate, concentrated and separated by column chromatography to obtain a yellow solid. (1.1 g, yield: 81%).

### Step 2: Synthesis of 4,7-dichloro-8-fluoro-N-(4-methoxybenzyl)-2-(methylthio)pyrido[4,3-d]pyrimidin-5-amine

Compound 7-chloro-8-fluoro-5-((4-methoxybenzyl)amino)-2-(methylthio)pyrido[4,3-d]pyrimidin-4-ol (1.1 g, 2.9 mmol) and N,N-diethylaniline (1 ml) were stirred in POCl₃ (9 ml) at 110° C. overnight. After the reaction was complete, the reaction liquid was concentrated to give the crude product, used directly to the next step. (1.2 g, yield: 100%).

### Step 3: Synthesis of 4,7-dichloro-8-fluoro-2-(methylthio)pyrido[4,3-d]pyrimidin-5-amine

Compound 4,7-dichloro-8-fluoro-N-(4-methoxybenzyl)-2-(methylthio)pyrido[4,3-d]pyrimidin-5-amine (1.2 g, 2.9 mmol) was stirred in TFA (10 mL) at room temperature for 5 h. After the reaction was complete, the reaction liquid was concentrated, extracted with ethyl acetate and saturated sodium bicarbonate, and the organic phase was washed with a saturated aqueous NaCl solution, dried with anhydrous sodium sulfate, concentrated to give the crude product, used directly to the next step. (805 mg, yield: 100%).

### Step 4: Synthesis of 5,10-dichloro-6-fluoro-8-(methylthio)imidazo[1′,2′:1,2]pyrido[4,3-d]pyrimidine

Compound 4,7-dichloro-8-fluoro-2-(methylthio)pyrido[4,3-d]pyrimidin-5-amine (805 mg, 2.9 mmol) was stirred in i-PrOH (10 mL) and HBr (1 mL) at 90° C. for 3 h. After the reaction was complete, the reaction liquid was cooled to room temperature, concentrated, extracted with DCM and sodium bicarbonate, the organic phase was dried with anhydrous sodium sulfate, concentrated, purified by column chromatography to give a yellow solid (610 mg, yield: 70%).

### Steps 5-9: Synthesis of 4-(10-((1R,5S)-3,8-diazabicyclo[3.2.1]octan-3-yl)-6-fluoro-8-(((2R,7aS)-2-fluorotetrahydro-1H-pyrrolizin-7a(5H)-yl)methoxy)imidazo[1′,2′:1,2]pyrido[4,3-d]pyrimidin-5-yl)-5-ethynyl-6-fluoronaphthalen-2-ol

Yellow solid (13 mg) was obtained by using the method from step 11 to step 15 of example 387. ¹H NMR (400 MHz, Chloroform) δ 8.69 (d, J=2.9 Hz, 1H), 7.98 (d, J=15.0 Hz, 1H), 7.68 (ddd, J=15.0, 10.0, 3.0 Hz, 1H), 7.57 (t, J=3.0 Hz, 1H), 7.45 (d, J=15.0 Hz, 1H), 6.89 (dd, J=16.0, 15.1 Hz, 1H), 5.82 (tt, J=13.9, 8.4 Hz, 1H), 5.64 (tt, J=13.7, 8.3 Hz, 1H), 5.11 (dd, J=24.8, 11.1 Hz, 2H), 5.00 (s, 1H), 4.56 (d, J=24.9 Hz, 1H), 4.26 (d, J=24.7 Hz, 1H), 3.95-3.71 (m, 3H), 3.34-3.15 (m, 3H), 3.01-2.82 (m, 2H), 2.65 (ddd, J=50.4, 24.7, 8.3 Hz, 1H), 2.21-2.05 (m, 2H), 1.91 (ddd, J=50.4, 24.8, 13.8 Hz, 1H), 1.78-1.63 (m, 2H), 1.57-1.18 (m, 5H), 1.05 (s, 1H).

### Example 400: Synthesis of 4-(9-((1R,5S)-3,8-diazabicyclo[3.2.1]octan-3-yl)-7-(((2R,7aS)-2-fluorotetrahydro-1H-pyrrolizin-7a(5H)-yl)methoxy)-3-methyl-3H-imidazo[4,5-f]quinazolin-4-yl)-5-ethynyl-6-fluoronaphthalen-2-ol

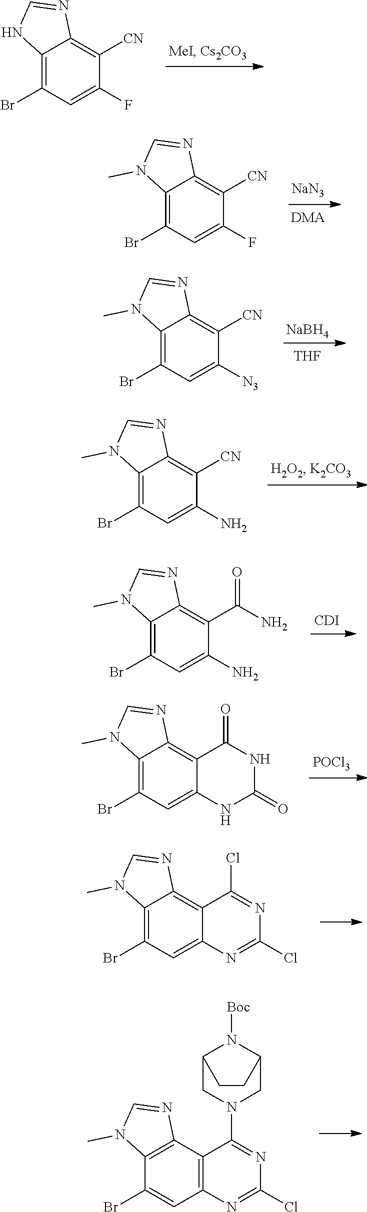

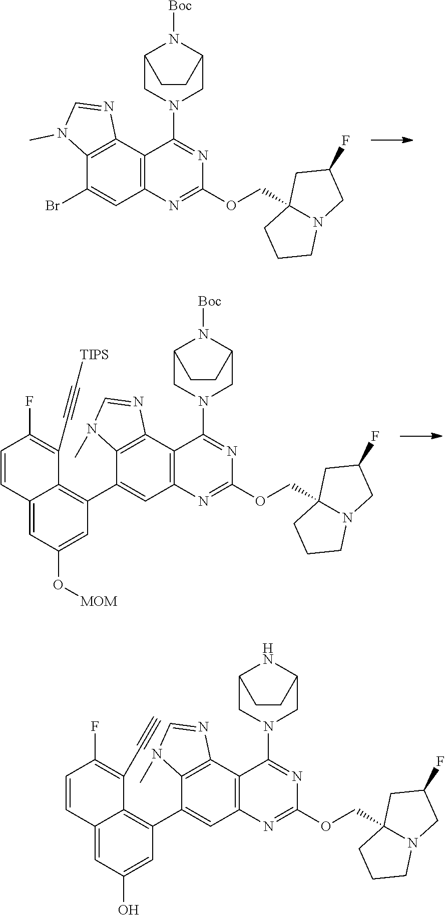

### Step 1: Synthesis of 7-bromo-5-fluoro-1-methyl-1H-benzo[d]imidazole-4-carbonitrile

Compound 7-bromo-5-fluoro-1H-benzo[d]imidazole-4-carbonitrile (1 g, 4.1 mmol), Mel (1.16 g, 8.2 mmol) and (2.67 g, 8.2 mmol) were stirred in DMF (10 mL) at room temperature for 24 h. After the reaction was complete, the mixture was diluted with water and extracted with ethyl acetate, and the organic phase was washed with a saturated aqueous NaCl solution, dried with anhydrous sodium sulfate, concentrated to give the crude product, used directly to the next step. (1.1 g, yield: 100%).

### Step 2: Synthesis of 5-azido-7-bromo-1-methyl-1H-benzo[d]imidazole-4-carbonitrile

Compound 7-bromo-5-fluoro-1-methyl-1H-benzo[d]imidazole-4-carbonitrile (1.1 g, 4.1 mmol) and NaN₃ (320 mg, 4.9 mmol) were stirred in DMA (10 mL) at 90° C. for 8 h. After the reaction was complete, the mixture was diluted with water and extracted with ethyl acetate, and the organic phase was washed with a saturated aqueous NaCl solution, dried with anhydrous sodium sulfate, concentrated and separated by column chromatography to obtain a yellow solid. (750 mg, yield: 63%).

### Step 3: Synthesis of 5-amino-7-bromo-1-methyl-1H-benzo[d]imidazole-4-carbonitrile

Compound 5-azido-7-bromo-1-methyl-1H-benzo[d]imidazole-4-carbonitrile (750 mg, 2.7 mmol) in THF (20 mL) under ice bath cooling was added NaBH₄ (103 mg, 2.7 mmol). The mixture was stirred at room temperature for 5 h. After the reaction was complete, the reaction was quenched with water, extracted with ethyl acetate, and the organic phase was washed with a saturated aqueous NaCl solution, dried with anhydrous sodium sulfate, concentrated and separated by column chromatography to obtain a yellow solid. (620 mg, yield: 91%).

### Step 4: Synthesis of 5-amino-7-bromo-1-methyl-1H-benzo[d]imidazole-4-carboxamide

Compound 5-amino-7-bromo-1-methyl-1H-benzo[d]imidazole-4-carbonitrile (620 mg, 2.5 mmol) in DMSO (10 mL) under ice bath cooling was added K₂CO₃(1 g, 7.5 mmol) and H₂O₂(2 mL). The mixture was stirred at room temperature for 5 h. After the reaction was complete, the reaction was diluted with cold water, and solid was precipitated out. The mixture was filtered, washed with water, dried in vacuum to give a yellow solid. (640 mg, yield: 96%).

### Step 5: Synthesis of 4-bromo-3-methyl-3,6-dihydro-7H-imidazo[4,5-f]quinazoline-7,9(8H)-dione

Compound 5-amino-7-bromo-1-methyl-1H-benzo[d]imidazole-4-carboxamide (640 mg, 2.4 mmol), K₂CO₃ (662 mg, 4.8 mmol) and CDI (778 mg, 4.8 mmol) were stirred in DMF (10 mL) at 90° C. for 4 h. After the reaction was complete, the reaction was diluted with water, extracted with ethyl acetate, pH of the water phase was adjusted to 2-3 with 10% HCl, and solid was precipitated out. The mixture was filtered, dried in vacuum to give a yellow solid. (620 mg, yield: 88%).

### Step 6: Synthesis of 4-bromo-7,9-dichloro-3-methyl-3H-imidazo[4,5-f]quinazoline

Compound 4-bromo-3-methyl-3,6-dihydro-7H-imidazo[4,5-f]quinazoline-7,9(8H)-dione (620 mg, 2.1 mmol) and N,N-diethylaniline (1 ml) were stirred in POCl₃ (9 ml) at 110° C. overnight. After the reaction was complete, the reaction liquid was concentrated, purified by column chromatography (PE) to give a yellow solid. (530 mg, 76%).

### Step 7: Synthesis of tert-butyl (1R,5S)-3-(4-bromo-7-chloro-3-methyl-3H-imidazo[4,5-f]quinazolin-9-yl)-3,8-diazabicyclo[3.2.1]octane-8-carboxylate

Compound 4-bromo-7,9-dichloro-3-methyl-3H-imidazo[4,5-f]quinazoline (530 mg, 1.6 mmol) and tert-butyl (1R,5S)-3,8-diazabicyclo[3.2.1]octane-8-carboxylate (360 mg, 1.7 mmol) in anhydrous DMF (8 mL) under ice bath cooling, was added DIEA (310 mg, 2.4 mmol) and stirred for 30 min. After the reaction was complete, the reaction liquid was diluted with water and extracted with ethyl acetate, and the organic phase was washed with a saturated aqueous NaCl solution, dried with anhydrous sodium sulfate, concentrated to obtain a light yellow solid, washed with PE, filtered, dried to give a light yellow solid (570 mg, yield: 70%).

### Step 8: Synthesis of tert-butyl (1R,5S)-3-(4-bromo-7-(((2R,7aS)-2-fluorotetrahydro-1H-pyrrolizin-7a(5H)-yl)methoxy)-3-methyl-3H-imidazo[4,5-f]quinazolin-9-yl)-3,8-diazabicyclo[3.2.1]octane-8-carboxylate

The compound ((2R,7aS)-2-fluorotetrahydro-1H-pyrrolizin-7a(5H)-yl)methanol (220 mg, 1.38 mmol) was dissolved in anhydrous tetrahydrofuran (5 mL), 60% NaH (51 mg, 1.27 mmol) was added under ice-water bath cooling condition and reacted under stirring for 20 min, and tert-butyl (1R,5S)-3-(4-bromo-7-chloro-3-methyl-3H-imidazo[4,5-f]quinazolin-9-yl)-3,8-diazabicyclo[3.2.1]octane-8-carboxylate (570 mg, 1.1 mmol) was added and reacted under stirring for 4 h. After the reaction was complete, the reaction was quenched with cold water, extracted with ethyl acetate, and the organic phase was washed with a saturated aqueous NaCl solution, dried with anhydrous sodium sulfate, concentrated and separated by column chromatography to obtain an off-white solid. (360 mg, yield: 51%).

### Steps 9 to 10: Synthesis of 4-(9-((1R,5S)-3,8-diazabicyclo[3.2.1]octan-3-yl)-7-(((2R,7aS)-2-fluorotetrahydro-1H-pyrrolizin-7a(5H)-yl)methoxy)-3-methyl-3H-imidazo[4,5-f]quinazolin-4-yl)-5-ethynyl-6-fluoronaphthalen-2-ol

Yellow solid was obtained by using the synthetic method from steps 6 to 7 of example 327. (15 mg, 0.0056 mmol, total yield: 4.8%). ¹H NMR (400 MHz, Chloroform) δ 8.56 (s, 1H), 8.05 (s, 1H), 7.85-7.77 (m, 2H), 7.49 (t, J=3.0 Hz, 1H), 7.32 (dd, J=16.0, 3.0 Hz, 1H), 6.90 (ddd, J=15.8, 15.2, 2.9 Hz, 1H), 5.40 (p, J=4.7 Hz, 1H), 5.21 (p, J=4.7 Hz, 1H), 5.02 (s, 1H), 4.57-4.38 (m, 3H), 4.23 (d, J=24.7 Hz, 1H), 3.92-3.73 (m, 7H), 3.32-3.07 (m, 2H), 2.90 (ddd, J=50.4, 24.8, 4.7 Hz, 1H), 2.70 (dt, J=24.6, 12.1 Hz, 1H), 2.50-2.34 (m, 2H), 2.13 (ddd, J=50.4, 24.8, 4.6 Hz, 1H), 1.91-1.75 (m, 2H), 1.68-1.43 (m, 2H), 1.33-1.19 (m, 3H), 1.02 (s, 1H).

### Example 401: Synthesis of 4-(10-((1R,5S)-3,8-diazabicyclo[3.2.1]octan-3-yl)-6-fluoro-8-(((2R,7aS)-2-fluorotetrahydro-1H-pyrrolizin-7a(5H)-yl)methoxy)-2,3-dihydroimidazo[1′,2′:1,2]pyrido[4,3-d]pyrimidin-5-yl)naphthalen-2-ol

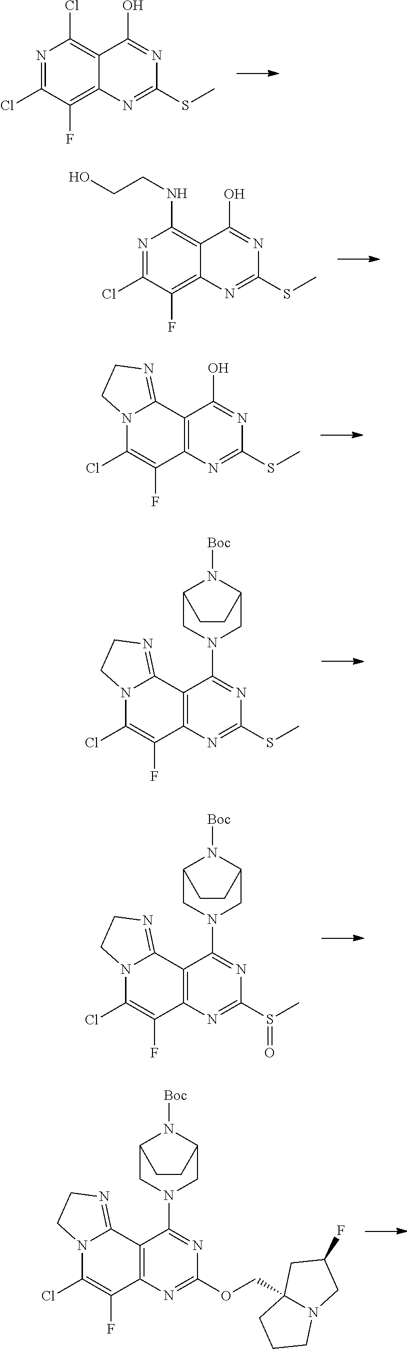

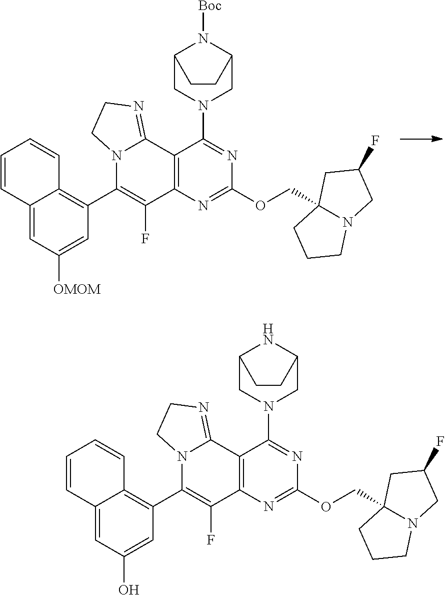

### Step 1: Synthesis of 7-chloro-8-fluoro-5-((2-hydroxyethyl)amino)-2-(methylthio)pyrido[4,3-d]pyrimidin-4-ol

Compound 5,7-dichloro-8-fluoro-2-(methylthio)pyrido[4,3-d]pyrimidin-4-ol (200 mg, 0.71 mmol), 2-aminoethan-1-ol (66 mg, 1.07 mmol) and Cs₂CO₃ (698 mg, 2.14 mmol) were stirred in DMA (10 mL) at 100° C. for 12 h. After the reaction was completed, cooled to room temperature, the mixture was diluted with water, extracted with ethyl acetate. The organic phase was dried with anhydrous sodium sulfate, concentrated under reduced pressure to obtain the crude product, used directly in the next step. (160 mg, yield: 73.5%). MS m/z: 305.6 [M+H]⁺

### Step 2: Synthesis of 5-chloro-6-fluoro-8-(methylthio)-2,3-dihydroimidazo[1′,2′:1,2]pyrido[4,3-d]pyrimidin-10-ol

Compound 7-chloro-8-fluoro-5-((2-hydroxyethyl)amino)-2-(methylthio)pyrido[4,3-d]pyrimidin-4-ol (160 mg, 0.53 mmol) in CHCl₃ (6 mL), was added SOCl₂ (2 mL) and stirred at 70° C. for 12 h. After the reaction was completed, the mixture was concentrated, diluted with saturated sodium bicarbonate aqueous solution, filtered to obtained the solid, washed with water, dried to give an off-white solid (130 mg, yield: 86.3%). MS m/z: 287 [M+H]⁺

### Step 3: Synthesis of tert-butyl (1R,5S)-3-(5-chloro-6-fluoro-8-(methylthio)-2,3-dihydroimidazo[1′,2′:1,2]pyrido[4,3-d]pyrimidin-10-yl)-3,8-diazabicyclo[3.2.1]octane-8-carboxylate

Compound 5-chloro-6-fluoro-8-(methylthio)-2,3-dihydroimidazo[1′,2′:1,2]pyrido[4,3-d]pyrimidin-10-ol (120 mg, 0.42 mmol), DIEA (81 mg, 0.63 mmol) and POCl₃ (77 mg, 0.50 mmol) were stirred in CH₃CN (10 ml) at 80° C. for 1.5 h. After the reaction was complete, DIEA (81 mg, 0.63 mmol) and tert-butyl (1R,5S)-3,8-diazabicyclo[3.2.1]octane-8-carboxylate (89 mg, 0.42 mmol) were added to the mixture under 0-10° C., stirred at room temperature for 30 min. After the reaction was completed, the mixture was diluted with water, extracted with ethyl acetate. The organic phase was dried with anhydrous sodium sulfate, concentrated to give a yellow solid, beating with petroleum ether to give a white solid (160 mg, yield: 81.9%).

### Step 4: Synthesis of tert-butyl (1R,5S)-3-(5-chloro-6-fluoro-8-(methylsulfinyl)-2,3-dihydroimidazo[1′,2′:1,2]pyrido[4,3-d]pyrimidin-10-yl)-3,8-diazabicyclo[3.2.1]octane-8-carboxylate

Compound tert-butyl (1R,5S)-3-(5-chloro-6-fluoro-8-(methylthio)-2,3-dihydroimidazo[1′,2′:1,2]pyrido[4,3-d]pyrimidin-10-yl)-3,8-diazabicyclo[3.2.1]octane-8-carboxylate (160 mg, 0.33 mmol) in DCM (10 mL) at 0° C., was added 85% m-CPBA (102 mg, 0.50 mmol) and stirred for 30 min. After the reaction was complete, the reaction was quenched by saturated sodium bisulfite solution and extracted with dichloromethane, and the organic phase was washed with saturated sodium bicarbonate and brine, dried with anhydrous sodium sulfate and concentrated to obtain a white solid, used directly in the next step (165 mg, yield: 100%).

### Step 5: Synthesis of tert-butyl (1R,5S)-3-(5-chloro-6-fluoro-8-(((2R,7aS)-2-fluorotetrahydro-1H-pyrrolizin-7a(5H)-yl)methoxy)-2,3-dihydroimidazo[1′,2′:1,2]pyrido[4,3-d]pyrimidin-10-yl)-3,8-diazabicyclo[3.2.1]octane-8-carboxylate

The compound ((2R,7aS)-2-fluorotetrahydro-1H-pyrrolizin-7a(5H)-yl)methanol (67 mg, 0.42 mmol) was dissolved in anhydrous tetrahydrofuran (5 mL), 60% NaH (10 mg, 0.39 mmol) was added under ice-water bath cooling condition and reacted under stirring for 20 min, and tert-butyl (1R,5S)-3-(5-chloro-6-fluoro-8-(methylsulfinyl)-2,3-dihydroimidazo[1′,2′:1,2]pyrido[4,3-d]pyrimidin-10-yl)-3,8-diazabicyclo[3.2.1]octane-8-carboxylate (160 mg, 0.32 mmol) was added and reacted under stirring at room temperature for 8 h. After the reaction was complete, the reaction was quenched with cold water, extracted with ethyl acetate, and the organic phase was washed with a saturated aqueous NaCl solution, dried with anhydrous sodium sulfate, concentrated and separated by column chromatography (DCM/MeOH=30/1) to obtain an off-white solid. (100 mg, yield: 52.5%).

### Step 6: Synthesis of tert-butyl (1R,5S)-3-(6-fluoro-8-(((2R,7aS)-2-fluorotetrahydro-1H-pyrrolizin-7a(5H)-yl)methoxy)-5-(3-(methoxymethoxy)naphthalen-1-yl)-2,3-dihydroimidazo[1′,2′:1,2]pyrido[4,3-d]pyrimidin-10-yl)-3,8-diazabicyclo[3.2.1]octane-8-carboxylate

Compound tert-butyl (1R,5S)-3-(5-chloro-6-fluoro-8-(((2R,7aS)-2-fluorotetrahydro-1H-pyrrolizin-7a(5H)-yl)methoxy)-2,3-dihydroimidazo[1′,2′:1,2]pyrido[4,3-d]pyrimidin-10-yl)-3,8-diazabicyclo[3.2.1]octane-8-carboxylate (20 mg, 0.03 mmol), 2-(3-(methoxymethoxy)naphthalen-1-yl)-4,4,5,5-tetramethyl-1,3,2-dioxaborolane (13 mg, 0.04 mmol), Cs₂CO₃ (22 mg, 0.07 mmol) and Pd(PPh₃)₄ (20 mg, 0.02 mmol) were stirred in 1,4-dioxane/H₂O (5 ml/1 ml) under Ar at 100° C. for 4 h. After the reaction was complete, the mixture was concentrated to give the crude product, purified by prep-TLC to give an light yellow solid. (20 mg, yield: 79.6%). MS m/z: 744.3 [M+H]⁺.

### Step 7: Synthesis of 4-(10-((1R,5S)-3,8-diazabicyclo[3.2.1]octan-3-yl)-6-fluoro-8-(((2R,7aS)-2-fluorotetrahydro-1H-pyrrolizin-7a(5H)-yl)methoxy)-2,3-dihydroimidazo[1′,2′:1,2]pyrido[4,3-d]pyrimidin-5-yl)naphthalen-2-ol

Compound tert-butyl (1R,5S)-3-(6-fluoro-8-(((2R,7aS)-2-fluorotetrahydro-1H-pyrrolizin-7a(5H)-yl)methoxy)-5-(3-(methoxymethoxy)naphthalen-1-yl)-2,3-dihydroimidazo[1′,2′:1,2]pyrido[4,3-d]pyrimidin-10-yl)-3,8-diazabicyclo[3.2.1]octane-8-carboxylate (20 mg, 0.026 mmol) was stirred in CH₃CN (2 mL) and HCl/dioxane (1 mL, 4M) for 30 min at room temperature. After the reaction was complete, pH of the solution was adjusted to 7-8 with saturated sodium bicarbonate aqueous solution, extracted with ethyl acetate, and the organic phase was dried with anhydrous sodium sulfate, concentrated, purified by prep-TLC (DCM/MeOH(7N NH₃)=15/1) to give a white solid (10 mg, yield: 62.02%). MS m/z: 600.6 [M+H]⁺

Examples 390-426 were obtained by using the method of Example 386, 387, 388, 389.

| | | | m/z: ES⁺ |
|---|---|---|---|
| Ex. | Compound name | Structural formula | [M + H]⁺ |
| 402 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-5-ethynyl-6-fluoronaphthalen-2-ol | 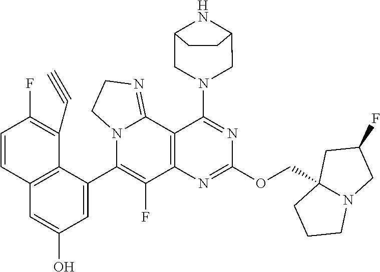 [SMILES: C#Cc1c(F)ccc2cc(O)cc(C3=C(F)c4nc(OC[C@@]56CCCN5C[C@H](F)C6)nc(N5CC6CCC(C5)N6)c4C4=NCCN43)c12] | 642.7 |
| 403 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-5-ethyl-6-fluoronaphthalen-2-ol | 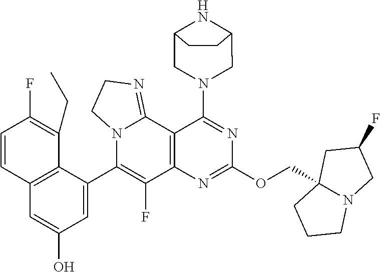 [SMILES: CCc1c(F)ccc2cc(O)cc(C3=C(F)c4nc(OC[C@@]56CCCN5C[C@H](F)C6)nc(N5CC6CCC(C5)N6)c4C4=NCCN43)c12] | 646.7 |
| 404 | 8-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-1-ethynylisoquinolin-6-ol | 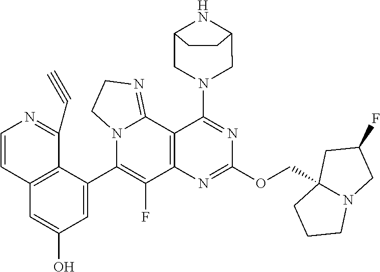 [SMILES: C#Cc1nccc2cc(O)cc(C3=C(F)c4nc(OC[C@@]56CCCN5C[C@H](F)C6)nc(N5CC6CCC(C5)N6)c4C4=NCCN43)c12] | 625.7 |
| 405 | 8-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-1-ethylisoquinolin-6-ol | 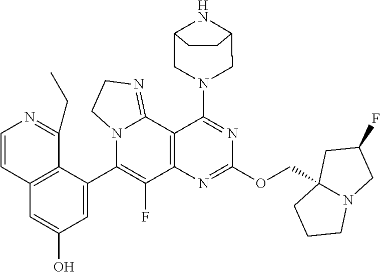 [SMILES: CCc1nccc2cc(O)cc(C3=C(F)c4nc(OC[C@@]56CCCN5C[C@H](F)C6)nc(N5CC6CCC(C5)N6)c4C4=NCCN43)c12] | 629.7 |
| 406 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-5,6,7,8-tetrahydronaphthalen-2-ol | 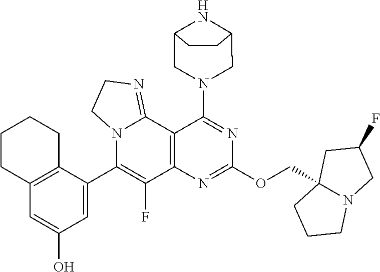 [SMILES: Oc1cc2c(c(C3=C(F)c4nc(OC[C@@]56CCCN5C[C@H](F)C6)nc(N5CC6CCC(C5)N6)c4C4=NCCN43)c1)CCCC2] | 604.7 |
| 407 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-5-methyl-5,6,7,8-tetrahydronaphthalen-2- ol | 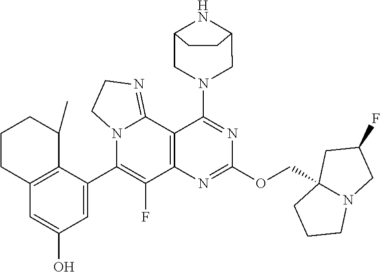 [SMILES: CC1CCCc2cc(O)cc(C3=C(F)c4nc(OC[C@@]56CCCN5C[C@H](F)C6)nc(N5CC6CCC(C5)N6)c4C4=NCCN43)c21] | 618.7 |
| 408 | 7-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-1-ethyl-2,3-dihydro-1H-inden-5-ol | 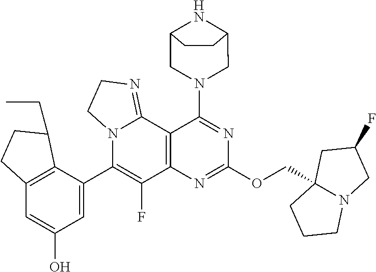 [SMILES: CCC1CCc2cc(O)cc(C3=C(F)c4nc(OC[C@@]56CCCN5C[C@H](F)C6)nc(N5CC6CCC(C5)N6)c4C4=NCCN43)c21] | 618.7 |
| 409 | 5-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-6-ethyl-1,1a,6,6a- tetrahydrocyclopropa[a]inden-3-ol | 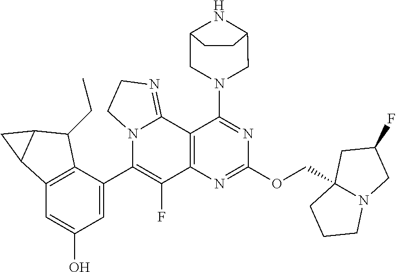 [SMILES: CCC1c2c(C3=C(F)c4nc(OC[C@@]56CCCN5C[C@H](F)C6)nc(N5CC6CCC(C5)N6)c4C4=NCCN43)cc(O)cc2C2CC21] | 630.7 |
| 410 | 8-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-1-methylisochroman-6-ol | 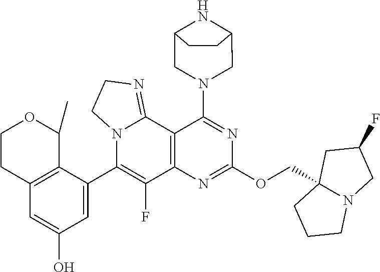 [SMILES: CC1OCCc2cc(O)cc(C3=C(F)c4nc(OC[C@@]56CCCN5C[C@H](F)C6)nc(N5CC6CCC(C5)N6)c4C4=NCCN43)c21] | 620.7 |
| 411 | 5-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-3-ethylimidazo[1,5-a]pyridin-7-ol | 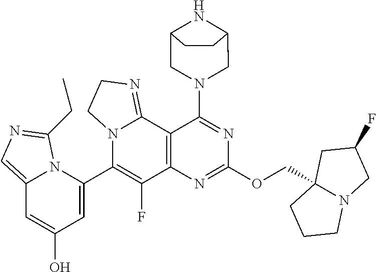 [SMILES: CCc1ncc2cc(O)cc(C3=C(F)c4nc(OC[C@@]56CCCN5C[C@H](F)C6)nc(N5CC6CCC(C5)N6)c4C4=NCCN43)n12] | 618.7 |
| 412 | 5-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)-2,3-dihydroimidazo[1′,2′:1,2]pyrido[4,3- d]pyrimidin-5-yl)-3-ethylindolizin-7-ol | 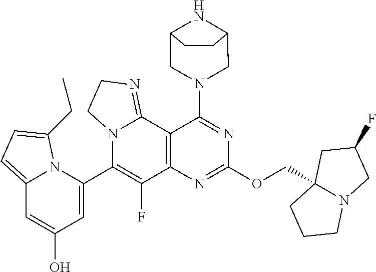 [SMILES: CCc1ccc2cc(O)cc(C3=C(F)c4nc(OC[C@@]56CCCN5C[C@H](F)C6)nc(N5CC6CCC(C5)N6)c4C4=NCCN43)n12] | 617.7 |
| 413 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)imidazo[1′,2′:1,2]pyrido[4,3-d]pyrimidin-5-yl)- 5-ethyl-6-fluoronaphthalen-2-ol | 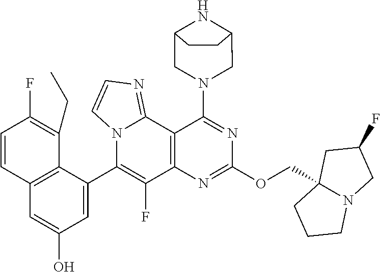 [SMILES: CCc1c(F)ccc2cc(O)cc(-c3c(F)c4nc(OC[C@@]56CCCN5C[C@H](F)C6)nc(N5CC6CCC(C5)N6)c4c4nccn34)c12] | 644.72 |
| 414 | 4-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)imidazo[1′,2′:1,2]pyrido[4,3-d]pyrimidin-5-yl)- 5-methyl-5,6,7,8-tetrahydronaphthalen-2-ol | 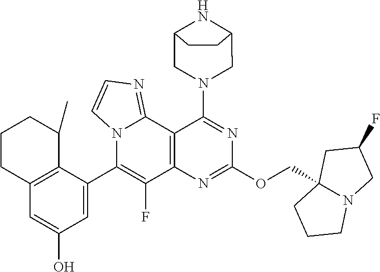 [SMILES: CC1CCCc2cc(O)cc(-c3c(F)c4nc(OC[C@@]56CCCN5C[C@H](F)C6)nc(N5CC6CCC(C5)N6)c4c4nccn34)c21] | 616.7 |
| 415 | 7-(10-((1R,5S)-3,8-diazabicyclo[3.2.1]octan-3-yl)-6-fluoro- 8-(((2R,7aS)-2-fluorotetrahydro-1H-pyrrolizin-7a(5H)- yl)methoxy)imidazo[1′,2′:1,2]pyrido[4,3-d]pyrimidin-5-yl)- 1-ethyl-2,3-dihydro-1H-inden-5-ol | 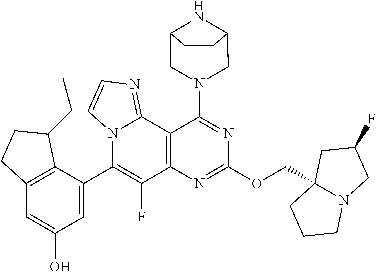 [SMILES: CCC1CCc2cc(O)cc(-c3c(F)c4nc(OC[C@@]56CCCN5C[C@H](F)C6)nc(N5CC6CCC(C5)N6)c4c4nccn34)c21] | 616.7 |
| 416 | 4-(1-((1R,5S)-3,8-diazabicyclo[3.2.1]octan-3-yl)-5-fluoro-3- (((2R,7aS)-2-fluorotetrahydro-1H-pyrrolizin-7a(5H)- yl)methoxy)imidazo[1′,5′:1,2]pyrido[4,3-d]pyrimidin-6-yl)- 5-ethyl-6-fluoronaphthalen-2-ol | 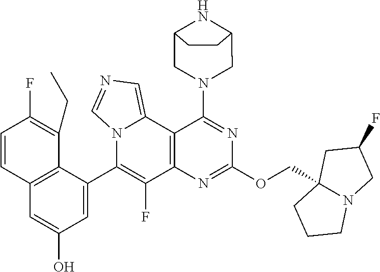 [SMILES: CCc1c(F)ccc2cc(O)cc(-c3c(F)c4nc(OC[C@@]56CCCN5C[C@H](F)C6)nc(N5CC6CCC(C5)N6)c4c4cncn34)c12] | 644.7 |
| 417 | 4-(1-((1R,5S)-3,8-diazabicyclo[3.2.1]octan-3-yl)-5-fluoro-3- (((2R,7aS)-2-fluorotetrahydro-1H-pyrrolizin-7a(5H)- yl)methoxy)imidazo[1′,5′:1,2]pyrido[4,3-d]pyrimidin-6-yl)- 5-methyl-5,6,7,8-tetrahydronaphthalen-2-ol | 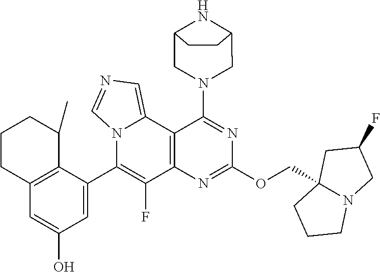 [SMILES: CC1CCCc2cc(O)cc(-c3c(F)c4nc(OC[C@@]56CCCN5C[C@H](F)C6)nc(N5CC6CCC(C5)N6)c4c4cncn34)c21] | 616.7 |
| 418 | 7-(1-((1R,5S)-3,8-diazabicyclo[3.2.1]octan-3-yl)-5-fluoro-3- (((2R,7aS)-2-fluorotetrahydro-1H-pyrrolizin-7a(5H)- yl)methoxy)imidazo[1′,5′:1,2]pyrido[4,3-d]pyrimidin-6-yl)- 1-ethyl-2,3-dihydro-1H-inden-5-ol | 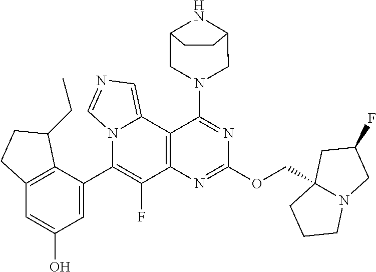 [SMILES: CCC1CCc2cc(O)cc(-c3c(F)c4nc(OC[C@@]56CCCN5C[C@H](F)C6)nc(N5CC6CCC(C5)N6)c4c4cncn34)c21] | 616.7 |
| 419 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5,6,7,8- tetrahydronaphthalen-2-ol | 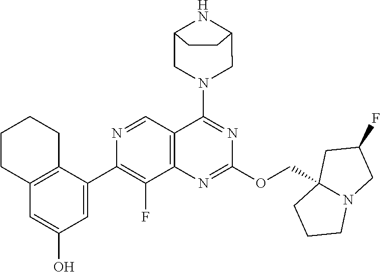 [SMILES: Oc1cc2c(c(-c3ncc4c(N5CC6CCC(C5)N6)nc(OC[C@@]56CCCN5C[C@H](F)C6)nc4c3F)c1)CCCC2] | 563.6 |
| 420 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5,6,7,8-tetrahydronaphthalen- 2-ol | 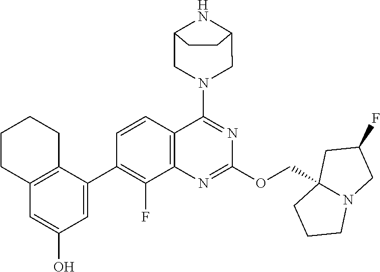 [SMILES: Oc1cc2c(c(-c3ccc4c(N5CC6CCC(C5)N6)nc(OC[C@@]56CCCN5C[C@H](F)C6)nc4c3F)c1)CCCC2] | 562.6 |
| 421 | 4-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)pyrido[4,3-d]pyrimidin-7-yl)-5-methyl- 5,6,7,8-tetrahydronaphthalen-2-ol | 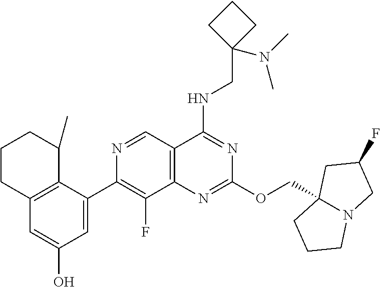 [SMILES: CC1CCCc2cc(O)cc(-c3ncc4c(NCC5(N(C)C)CCC5)nc(OC[C@@]56CCCN5C[C@H](F)C6)nc4c3F)c21] | 593.7 |
| 422 | 4-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)pyrido[4,3-d]pyrimidin-7-yl)-5-ethyl- 5,6,7,8-tetrahydronaphthalen-2-ol | 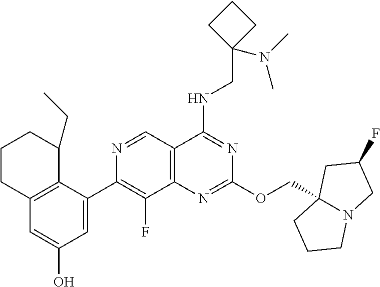 [SMILES: CCC1CCCc2cc(O)cc(-c3ncc4c(NCC5(N(C)C)CCC5)nc(OC[C@@]56CCCN5C[C@H](F)C6)nc4c3F)c21] | 607.7 |
| 423 | 7-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)pyrido[4,3-d]pyrimidin-7-yl)-1-ethyl- 2,3-dihydro-1H-inden-5-ol | 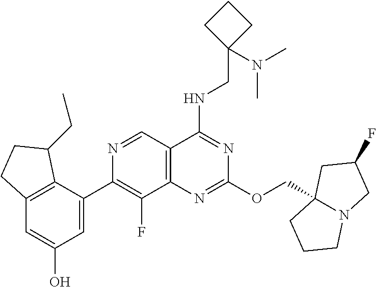 [SMILES: CCC1CCc2cc(O)cc(-c3ncc4c(NCC5(N(C)C)CCC5)nc(OC[C@@]56CCCN5C[C@H](F)C6)nc4c3F)c21] | 593.7 |
| 424 | 8-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)pyrido[4,3-d]pyrimidin-7-yl)-1- ethynylisoquinolin-6-ol | 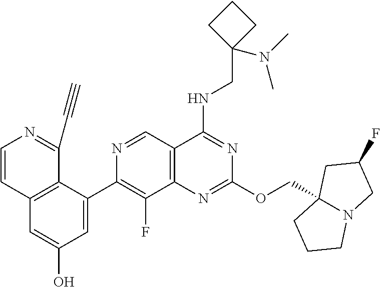 [SMILES: C#Cc1nccc2cc(O)cc(-c3ncc4c(NCC5(N(C)C)CCC5)nc(OC[C@@]56CCCN5C[C@H](F)C6)nc4c3F)c12] | 600.6 |
| 425 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5- (methylsulfonyl)naphthalen-2-ol | 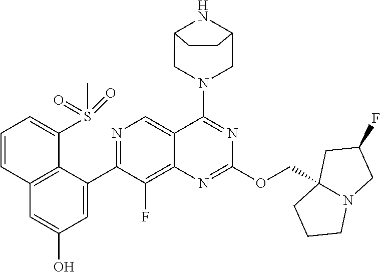 [SMILES: CS(=O)(=O)c1cccc2cc(O)cc(-c3ncc4c(N5CC6CCC(C5)N6)nc(OC[C@@]56CCCN5C[C@H](F)C6)nc4c3F)c12] | 637.7 |
| 426 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5-(methylthio)- 5,6,7,8-tetrahydronaphthalen-2-ol | 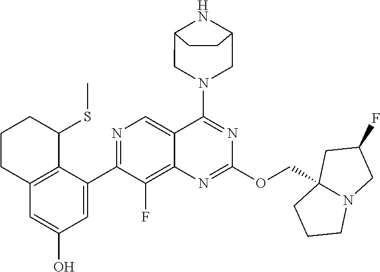 [SMILES: CSC1CCCc2cc(O)cc(-c3ncc4c(N5CC6CCC(C5)N6)nc(OC[C@@]56CCCN5C[C@H](F)C6)nc4c3F)c21] | 609.7 |
| 427 | 4-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)quinazolin-7-yl)-5-methyl-5,6,7,8- tetrahydronaphthalen-2-ol | 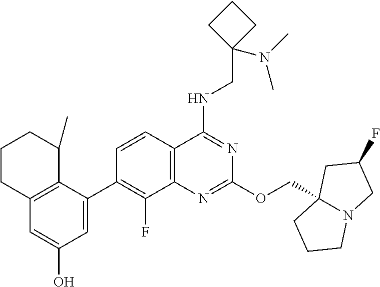 [SMILES: CC1CCCc2cc(O)cc(-c3ccc4c(NCC5(N(C)C)CCC5)nc(OC[C@@]56CCCN5C[C@H](F)C6)nc4c3F)c21] | 592.7 |
| 428 | 4-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)quinazolin-7-yl)-5-ethyl-5,6,7,8- tetrahydronaphthalen-2-ol | 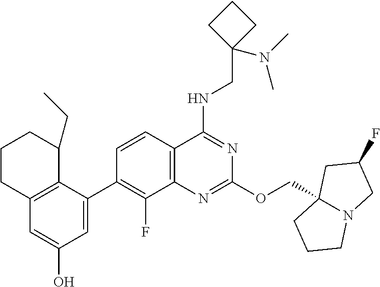 [SMILES: CCC1CCCc2cc(O)cc(-c3ccc4c(NCC5(N(C)C)CCC5)nc(OC[C@@]56CCCN5C[C@H](F)C6)nc4c3F)c21] | 606.7 |
| 429 | 7-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)quinazolin-7-yl)-1-ethyl-2,3-dihydro- 1H-inden-5-ol | 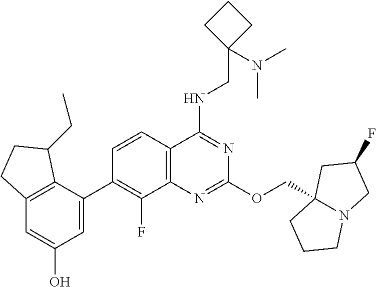 [SMILES: CCC1CCc2cc(O)cc(-c3ccc4c(NCC5(N(C)C)CCC5)nc(OC[C@@]56CCCN5C[C@H](F)C6)nc4c3F)c21] | 592.7 |
| 430 | 4-(4-(((1-(dimethylamino)cyclobutyl)methyl)amino)-8- fluoro-2-(((2R,7aS)-2-fluorotetrahydro-1H-pyrrolizin- 7a(5H)-yl)methoxy)quinazolin-7-yl)-5-ethynyl-6- fluoronaphthalen-2-ol | 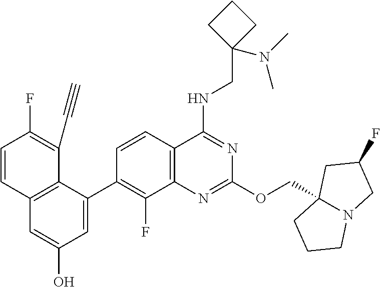 [SMILES: C#Cc1c(F)ccc2cc(O)cc(-c3ccc4c(NCC5(N(C)C)CCC5)nc(OC[C@@]56CCCN5C[C@H](F)C6)nc4c3F)c12] | 616.7 |
| 431 | 4-(4-((1R,5S)-3,8-diazabicyclo[3.2.1]octan-3-yl)-3,8- difluoro-2-(((2R,7aS)-2-fluorotetrahydro-1H-pyrrolizin- 7a(5H)-yl)methoxy)-1,6-naphthyridin-7-yl)-5-methyl- 5,6,7,8-tetrahydronaphthalen-2-ol | 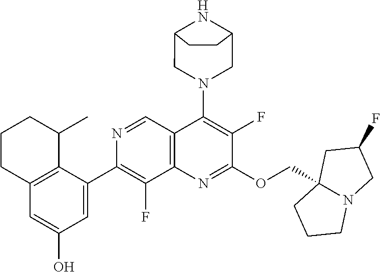 [SMILES: CC1CCCc2cc(O)cc(-c3ncc4c(N5CC6CCC(C5)N6)c(F)c(OC[C@@]56CCCN5C[C@H](F)C6)nc4c3F)c21] | 594.7 |
| 432 | 4-(4-((1R,5S)-3,8-diazabicyclo[3.2.1]octan-3-yl)-3,8- difluoro-2-(((2R,7aS)-2-fluorotetrahydro-1H-pyrrolizin- 7a(5H)-yl)methoxy)-1,6-naphthyridin-7-yl)-5-ethyl-5,6,7,8- tetrahydronaphthalen-2-ol | 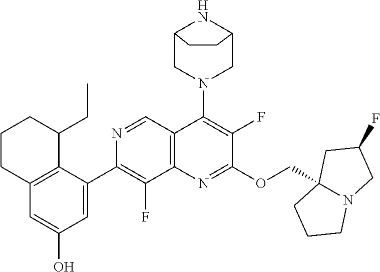 [SMILES: CCC1CCCc2cc(O)cc(-c3ncc4c(N5CC6CCC(C5)N6)c(F)c(OC[C@@]56CCCN5C[C@H](F)C6)nc4c3F)c21] | 608.7 |
| 433 | 7-(4-((1R,5S)-3,8-diazabicyclo[3.2.1]octan-3-yl)-3,8- difluoro-2-(((2R,7aS)-2-fluorotetrahydro-1H-pyrrolizin- 7a(5H)-yl)methoxy)-1,6-naphthyridin-7-yl)-1-methyl-2,3- dihydro-1H-inden-5-ol | 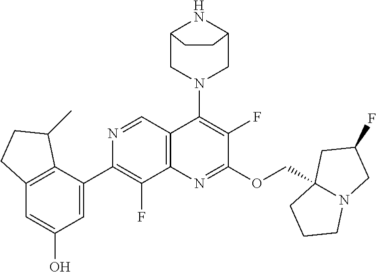 [SMILES: CC1CCc2cc(O)cc(-c3ncc4c(N5CC6CCC(C5)N6)c(F)c(OC[C@@]56CCCN5C[C@H](F)C6)nc4c3F)c21] | 580.4 |
| 434 | 7-(4-((1R,5S)-3,8-diazabicyclo[3.2.1]octan-3-yl)-3,8- difluoro-2-(((2R,7aS)-2-fluorotetrahydro-1H-pyrrolizin- 7a(5H)-yl)methoxy)-1,6-naphthyridin-7-yl)-1-ethyl-2,3- dihydro-1H-inden-5-ol | 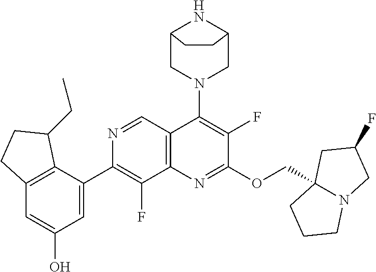 [SMILES: CCC1CCc2cc(O)cc(-c3ncc4c(N5CC6CCC(C5)N6)c(F)c(OC[C@@]56CCCN5C[C@H](F)C6)nc4c3F)c21] | 593.4 |
| 435 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)pyrido[4,3-d]pyrimidin-7-yl)-1a,2,3,7b- tetrahydro-1H-cyclopropa[a]naphthalen-6-ol | 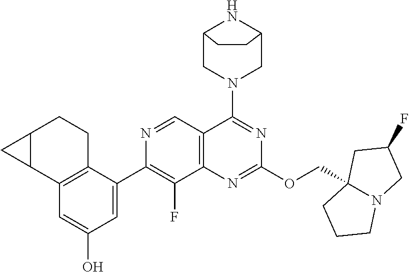 [SMILES: Oc1cc(-c2ncc3c(N4CC5CCC(C4)N5)nc(OC[C@@]45CCCN4C[C@H](F)C5)nc3c2F)c2c(c1)C1CC1CC2] | 575.6 |
| 436 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5,8-dimethyl- 5,6,7,8-tetrahydronaphthalen-2-ol | 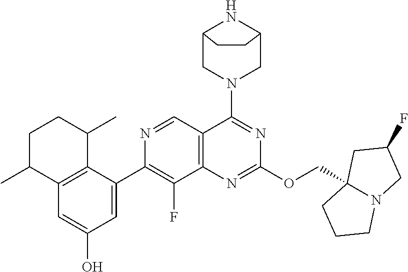 [SMILES: CC1CCC(C)c2c(-c3ncc4c(N5CC6CCC(C5)N6)nc(OC[C@@]56CCCN5C[C@H](F)C6)nc4c3F)cc(O)cc21] | 591.7 |
| 437 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-1a,2,3,7b-tetrahydro-1H- cyclopropa[a]naphthalen-6-ol | 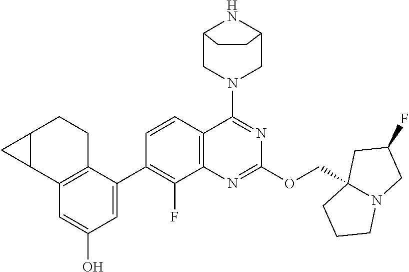 [SMILES: Oc1cc(-c2ccc3c(N4CC5CCC(C4)N5)nc(OC[C@@]45CCCN4C[C@H](F)C5)nc3c2F)c2c(c1)C1CC1CC2] | 574.7 |
| 438 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5,8-dimethyl-5,6,7,8- tetrahydronaphthalen-2-ol | 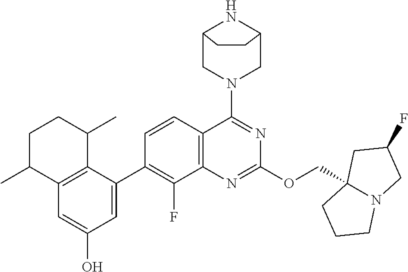 [SMILES: CC1CCC(C)c2c(-c3ccc4c(N5CC6CCC(C5)N6)nc(OC[C@@]56CCCN5C[C@H](F)C6)nc4c3F)cc(O)cc21] | 590.7 |
| 439 | 4-(4-((1R,5S)-3,8-diazabicyclo[3.2.1]octan-3-yl)-8-fluoro-2- ((1-(pyrrolidin-1-ylmethyl)cyclopropyl)methoxy)pyrido[4,3- d]pyrimidin-7-yl)-5-ethynyl-6-fluoronaphthalen-2-ol, S-axis chiral isomer | 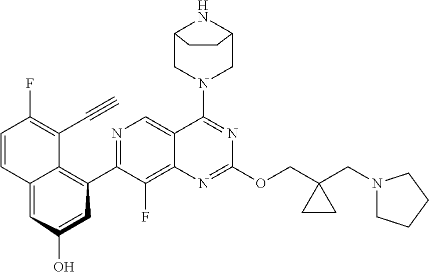 | 597.4 |
| 440 | 4-(4-((1R,5S)-3,8-diazabicyclo[3.2.1]octan-3-yl)-8-fluoro-2- ((1-(pyrrolidin-1-ylmethyl)cyclopropyl)methoxy)pyrido[4,3- d]pyrimidin-7-yl)-5-ethynyl-6-fluoronaphthalen-2-ol, R-axis chiral isomer | 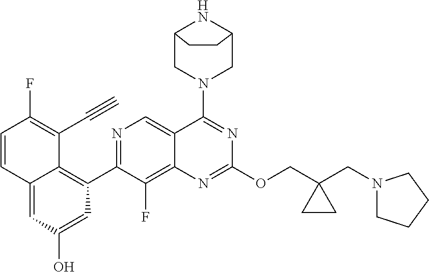 | 597.4 |
| 441 | 4-(4-((1R,5S)-3,8-diazabicyclo[3.2.1]octan-3-yl)-8-fluoro-2- ((1-(pyrrolidin-1-ylmethyl)cyclopropyl)methoxy)pyrido[4,3- d]pyrimidin-7-yl)-5-ethyl-6-fluoronaphthalen-2-ol, S-axis chiral isomer | 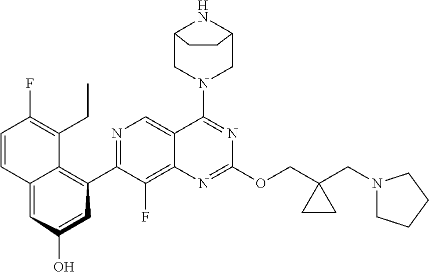 | 601.5 |
| 442 | 4-(4-((1R,5S)-3,8-diazabicyclo[3.2.1]octan-3-yl)-8-fluoro-2- ((1-(pyrrolidin-1-ylmethyl)cyclopropyl)methoxy)pyrido[4,3- d]pyrimidin-7-yl)-5-ethyl-6-fluoronaphthalen-2-ol, R-axis chiral isomer | 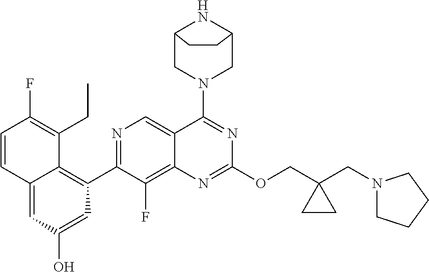 | 601.5 |
| 443 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5-ethynyl-6-fluoronaphthalen- 2-ol, R-axis chiral isomer | 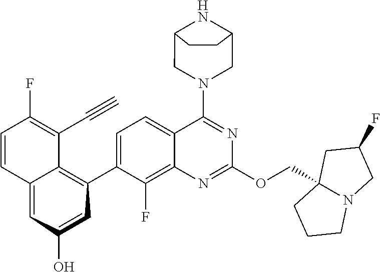 | 600.4 |
| 444 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5-ethynyl-6-fluoronaphthalen- 2-ol, S-axis chiral isomer | 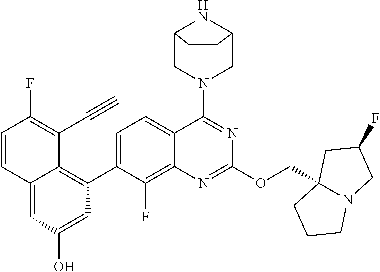 | 600.4 |
| 445 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5-ethyl-6-fluoronaphthalen-2- ol, R-axis chiral isomer | 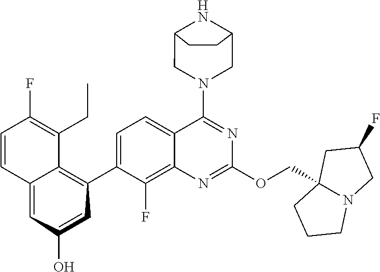 | 604.4 |
| 446 | 4-(4-((1R,5S)-3,8-diazabicyclo[3.2.1]octan-3-yl)-8-fluoro-2- (((2R,7aS)-2-fluorotetrahydro-1H-pyrrolizin-7a(5H)- yl)methoxy)quinazolin-7-yl)-5-ethyl-6-fluoronaphthalen-2- ol, S-axis chiral isomer | 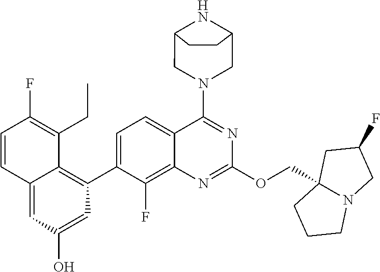 | 604.4 |

### Cell Activity Experiment

### 1. Cells

| Cell line | Culture medium formula | Cell mutation site |
|---|---|---|
| AGS | F12K + 10% FBS | KRAS^{G12D} |

The above cells were cultured in a complete medium containing 10% fetal bovine serum, 100 IU/mL penicillin and 100 jag/mL streptomycin, in the aseptic incubator with 95% relative humidity and 5% CO₂ concentration.

### 2. Reagents: F12K, D-PBS, FBS, Penicillin Streptomycin, Tryple, MTT (5 mg/mL)

### Experimental Method

### 1. Compound Preparation

Appropriate amount of compound powder was weighed in a 1.5 mL centrifuge tube, then the corresponding volume of dimethyl sulfoxide (DMSO) was added to form a 20 mM reserve solution. The solution was packed separately and stored in a refrigerator at −20° C. away from light.

### 2. Tumor Cell Culture

The cells were inoculated in a cell culture bottle, added an appropriate amount of complete medium, and cultured in a sterile incubator. When the degree of cell confluence reached more than 80%, subculture was carried out.

### 3. Detection of Cell Proliferation In Vitro by MTT/MTS Method

The adherent cells in logarithmic growth phase were digested by trypsin or the suspension cells were collected by centrifugation and counted. 90 μL cell suspension was inoculated into 96-well plate according to the cell inoculation density of 1000 cells per well, and 10 μL of 10-fold final concentration compound diluted by culture medium was added 24 hours later. Compared with the hole with the same volume of 5% DMSO as control, the final concentration of DMSO was 0.5%. After 3 days of drug treatment, the cell viability was detected by MTT. The specific methods are as follows: 10 μL MTT per well was added into the incubator for 4 hours, the supernatant was discarded and 150 μL DMSO was added to dissolve the crystal methylazan, and the absorbance of 490 nM was detected by enzyme labeling instrument, or 10 μL MITS per well was added to the incubator for 4 hours, and 490 nM absorbance was detected directly by enzyme labeling instrument. GraphPad Prism 6 software makes dose-effect curve and calculates IC50.

The IC50 values of formula (I) and (11) compound activity data are shown in Table 1:

| Ex No. | AGS IC₅₀((nM) | Ex No. | AGS IC₅₀((nM) |
|---|---|---|---|
| 1 | 8.1 | 2 | 3.0 |
| 3 | 4.5 | 4 | 11 |
| 5 | 3.8 | 6 | 14 |
| 7 | 2584 | 8 | >1000 |
| 9 | >1000 | 10 | 17 |
| 11 | 390 | 12 | 316 |
| 13 | 320 | 14 | 330 |
| 15 | 320 | 16 | 315 |
| 17 | 4134 | 18 | >1000 |
| 19 | >1000 | 20 | 271 |
| 21 | 269 | 22 | 3948 |
| 23 | 6952 | 24 | 280 |
| 25 | 290 | 26 | 300 |
| 27 | 295 | 28 | >1000 |
| 29 | >2500 | 30 | >2500 |
| 31 | >2500 | 32 | >2500 |
| 33 | >2500 | 34 | >2500 |
| 35 | >2500 | 36 | >2500 |
| 37 | >2500 | 38 | >2500 |
| 39 | >2500 | 40 | >2500 |
| 41 | >2500 | 42 | 109 |
| 43 | 31 | 44 | 47 |
| 45 | 39 | 46 | 13 |
| 47 | 4535 | 48 | 5158 |
| 49 | >10000 | 50 | >10000 |
| 51 | 1655 | 52 | >2500 |
| 53 | >2500 | 54 | >2500 |
| 55 | >2500 | 56 | >2500 |
| 57 | >2500 | 58 | >2500 |
| 59 | >2500 | 60 | >2500 |
| 61 | >2500 | 62 | >2500 |
| 63 | 1575 | 64 | >10000 |
| 65 | >10000 | 66 | >10000 |
| 67 | >10000 | 68 | 1288 |
| 69 | 411 | 70 | >2500 |
| 71 | >2500 | 72 | >2500 |
| 73 | 16.9 | 74 | >2500 |
| 75 | >2500 | 76 | >2500 |
| 77 | >2500 | 78 | >2500 |
| 79 | >2500 | 80 | >2500 |
| 81 | >2500 | 82 | >2500 |
| 83 | >2500 | 84 | >10000 |
| 85 | >1000 | 86 | >10000 |
| 87 | >10000 | 88 | >10000 |
| 89 | 1.5 | 90 | 8922 |
| 91 | 1.3 | 92 | 2.7 |
| 93 | 10.1 | 94 | 1.5 |
| 95 | 13.5 | 96 | 7.9 |
| 97 | 18 | 98 | 129 |
| 99 | 8.8 | 100 | 21 |
| 101 | 9.4 | 102 | 33 |
| 103 | 29 | 104 | 57 |
| 105 | 80 | 106 | 120 |
| 107 | 105 | 108 | 40.5 |
| 109 | >2500 | 110 | >2500 |
| 111 | >2500 | 112 | >2500 |
| 113 | >2500 | 114 | >2500 |
| 115 | >2500 | 116 | >2500 |
| 117 | >2500 | 118 | >2500 |
| 119 | >2500 | 120 | >2500 |
| 121 | >2500 | 122 | >2500 |
| 123 | >2500 | 124 | >2500 |
| 125 | >2500 | 126 | >2500 |
| 127 | >2500 | 128 | >2500 |
| 129 | >2500 | 130 | >2500 |
| 131 | >2500 | 132 | >2500 |
| 133 | >2500 | 134 | 586 |
| 135 | 1202 | 136 | >2500 |
| 137 | >2500 | 138 | >2500 |
| 139 | >2500 | 140 | >2500 |
| 141 | >2500 | 142 | >2500 |
| 143 | >2500 | 144 | >2500 |
| 145 | >2500 | 146 | >2500 |
| 147 | >2500 | 148 | 318 |
| 149 | 320 | 150 | 330 |
| 151 | 335 | 152 | 318 |
| 153 | 190 | 154 | 195 |
| 155 | 215 | 156 | 40 |
| 157 | 330 | 158 | 235 |
| 159 | >2500 | 160 | >2500 |
| 161 | 236 | 162 | >2500 |
| 163 | >2500 | 164 | 300 |
| 165 | >2500 | 166 | >2500 |
| 167 | 310 | 168 | >2500 |
| 169 | >2500 | 170 | 1040 |
| 171 | 1238 | 172 | 1230 |
| 173 | 1450 | 174 | 1460 |
| 175 | 1330 | 176 | 1344 |
| 177 | 530 | 178 | 535 |
| 179 | 550 | 180 | 556 |
| 181 | 536 | 182 | 533 |
| 183 | >2500 | 184 | >2500 |
| 185 | 1350 | 186 | 1420 |
| 187 | 1410 | 188 | 1550 |
| 189 | 152 | 190 | 150 |
| 191 | 120 | 192 | 121 |
| 193 | 125 | 194 | 150 |
| 195 | >2500 | 196 | >2500 |
| 197 | >2500 | 198 | >2500 |
| 199 | >2500 | 200 | >2500 |
| 201 | >2500 | 202 | 7.5 |
| 203 | 17 | 204 | 11 |
| 205 | 18 | 206 | 20 |
| 207 | 25 | 208 | 20 |
| 209 | 35 | 210 | 65 |
| 211 | 55 | 212 | 419 |
| 213 | >2500 | 214 | >10000 |
| 215 | >2500 | 216 | 3482 |
| 217 | 835 | 218 | 980 |
| 219 | >2500 | 220 | 5.0 |
| 221 | 875 | 222 | 2.5 |
| 223 | 14 | 224 | 4.8 |
| 225 | 668 | 226 | 2.4 |
| 227 | 5.0 | 228 | 1090 |
| 229 | 2.5 | 242 | 34 |
| 243 | 18 | 244 | 59 |
| 245 | 25 | 246 | 109 |
| 247 | 6.1 | 248 | 6.7 |
| 249 | >2500 | 250 | >2500 |
| 251 | >2500 | 252 | >2500 |
| 253 | 42 | 254 | 462 |
| 255 | 157 | 256 | 303 |
| 257 | 1848 | 258 | >10000 |
| 259 | 2630 | 260 | 417 |
| 261 | 1930 | 262 | 305 |
| 263 | >10000 | 264 | >10000 |
| 265 | >10000 | 266 | 52 |
| 267 | 1049 | 268 | 3997 |
| 269 | 30 | 270 | 20 |
| 271 | 11 | 272 | 70 |
| 273 | >2500 | 274 | |
| 275 | | 276 | |
| 277 | | 278 | >10000 |
| 279 | >10000 | 280 | |
| 281 | 537 | 282 | 15 |
| 283 | 14 | 284 | 2200 |
| 285 | 7.0 | 286 | 29 |
| 287 | 2137 | 288 | 7.2 |
| 289 | 15 | 290 | 3406 |
| 291 | 7.2 | 292 | 40 |
| 293 | 121 | 294 | 65 |
| 295 | 183 | 296 | >2500 |
| 297 | >2500 | 298 | |
| 299 | 945 | 300 | 177 |
| 301 | 28 | 302 | 22 |
| 303 | 22 | 304 | 35 |
| 305 | 102 | 306 | 366 |
| 307 | | 308 | |
| 309 | | 310 | |
| 311 | | 312 | |
| 313 | | 314 | |
| 315 | | 316 | |
| 317 | | 318 | |
| 319 | | 320 | |
| 321 | | 322 | |
| 323 | | 324 | |
| 325 | | 326 | |
| 327 | 230 | 328 | 8906 |
| 329 | 260 | 330 | 195 |
| 331 | 823 | 332 | >2500 |
| 333 | 455 | 334 | 125 |
| 335 | 20.7 | 336 | 9.9 |
| 337 | | 338 | |
| 339 | 15 | 340 | 20 |
| 341 | 25 | 342 | |
| 343 | 17 | 344 | 35 |
| 345 | | 346 | |
| 347 | | 348 | 85 |
| 349 | | 350 | |
| 351 | 30 | 352 | |
| 353 | | 354 | 25 |
| 355 | 20 | 356 | 21 |
| 357 | | 358 | |
| 359 | 35 | 360 | 10 |
| 361 | | 362 | |
| 363 | 753 | 364 | 811 |
| 365 | 3638 | 366 | 2100 |
| 367 | 194 | 368 | 55 |
| 369 | 180 | 370 | 130 |
| 371 | 85 | 372 | 65 |
| 373 | 30 | 374 | 32 |
| 375 | | 376 | 100 |
| 377 | 120 | 378 | 160 |
| 379 | 90 | 380 | 95 |
| 381 | 75 | 382 | 45 |
| 383 | 50 | 384 | 212 |
| 385 | 459 | 386 | 508 |
| 387 | 481 | 388 | 493 |
| 389 | 180 | 390 | 150 |
| 391 | 100 | 392 | 110 |
| 393 | 70 | 394 | 75 |
| 395 | | 396 | |
| 397 | 70 | 398 | 50 |
| 399 | 65 | 400 | >2500 |
| 401 | 100 | 402 | 56 |
| 403 | 60 | 404 | |
| 405 | | 406 | 120 |
| 407 | 35 | 408 | 41 |
| 409 | 45 | 410 | 75 |
| 411 | | 412 | 35 |
| 413 | | 414 | 45 |
| 415 | 60 | 416 | 28 |
| 417 | 35 | 418 | 75 |
| 419 | 14 | 420 | 60 |
| 421 | 26 | 422 | 35 |
| 423 | 55 | 424 | 30 |
| 425 | | 426 | 15 |
| 427 | 55 | 428 | 70 |
| 429 | 75 | 430 | 35 |

1. A compound of formula (I) or (II), or a pharmaceutically acceptable salt, tautomer, prodrug or stereoisomer thereof, wherein the compounds of formula (I) and (II) are: 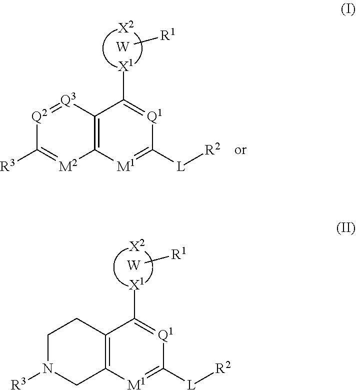
wherein: 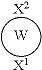
is a 4- to 12-membered saturated or partially saturated monocyclic, bridged cyclic, or spirocyclic ring, wherein the saturated or partially saturated monocyclic ring is optionally additionally substituted with one or more
R¹, X¹ is selected from N and CR⁴; X² is selected from NR⁴, CR⁴ and S(O)_{0,1,2}R⁴,
wherein,
R¹ is selected from hydrogen, halogen, C₁₋₆ alkyl optionally substituted by halogen or hydroxyl or —NR^{1a}R^{1b}, CN, —OR^{1a}, —SR^{1a}, —NR^{1a}R^{1b}, —S(O)R^{1a}, —S(O)₂R^{1a}, —C(O)R^{1a}, —C(O)OR^{1a}, —NR^{1a}C(O)R^{1b}, —C(O)NR^{1a}R^{1b}, —S(O)₂N(R^{1a}R^{1b})₂ and 5 to 6-membered heteroaryl, wherein R^{1a} and R^{1b} are each independently hydrogen, C₁₋₆ alkyl, hydroxy C₁₋₆ alkyl, halogenated C₁₋₆ alkyl and C₁₋₆ alkoxy C₁₋₆ alkyl, C₃₋₆ cycloalkyl;
R⁴ is selected from hydrogen, halogen, C₁₋₆ alkyl optionally substituted by halogen or hydroxyl, CN, —OR^{4a}, —SR^{4a}, —S(O)R^{4a}, —S(O)₂R^{4a}, —C(O)R^{4a}, —C(O)OR^{4a}, —NR^{4a}C(O)R^{4b}, —C(O)NR^{4a}R^{4b} and —S(O)₂N(R^{4a}R^{4b})₂, wherein R^{4a} and R^{4b} are each independently hydrogen, C₁₋₆ alkyl, hydroxy C₁₋₆ alkyl, halogenated C₁₋₆ alkyl and C₁₋₆ alkoxy C₁₋₆ alkyl;
L is a single bond, —O—, —S—, —NR^{La}—, —O—(CR^{La}R^{Lb})ₜ—, —S—(CR^{La}R^{Lb})ₜ—, —NR^{Lc}—(CR^{La}R^{Lb})ₜ—, —(CR^{La}R^{Lb})ₜ—O—, —(CR^{La}R^{Lb})ₜ—S—, —(CR^{La}R^{Lb})ₜ—NR^{Lc}—, —C(O)—, —SO₂—, —SO—, —C(O)—O—, —OC(O)—, —C(O)—NR^{Lc}— or —N^{Lc}C(O)—, wherein R^{La}, R^{Lb} and R^{Lc} are each independently hydrogen and C₁₋₆ alkyl, or R^{La} and R^{Lb} connected to the same carbon atom form C₃-C₆ cycloalkyl with the connected carbon atom, and t is an integer from 1 to 6;
R² is C₁₋₆ alkyl, C₂₋₆ alkenyl, C₂₋₆ alkynyl, C₃₋₈ cycloalkyl, aryl, heteroaryl, or heterocyclyl, wherein the C₁₋₆ alkyl, C₂₋₆ alkenyl, C₂₋₆ alkynyl, C₃₋₈ cycloalkyl, aryl, heteroaryl and heterocyclyl are each independently unsubstituted or substituted by one or more halogen, cyano, C₁₋₆ alkyl, halogenated C₁₋₆ alkyl, hydroxy C₁₋₆ alkyl, C₁₋₆ alkoxy C₁₋₆ alkyl, oxo, —OR^{2a}, —C(O)R^{2a}, —(CR^{2a}R^{2b})ₘ—OC(O)NR^{2c}R^{2d}, —CO₂R^{2a}, —CONR^{2c}R^{2d}, —NR^{2c}R^{2d}, —NR^{2c}R^{2d}, C₃₋₈ cycloalkyl, C₃₋₈ cycloalkyl C₁₋₆ alkyl, aryl, heteroaryl, and heterocyclyl, wherein R^{2a}, R^{2b}, R^{2c}, and R^{2d} are each independently hydrogen, C₁₋₆ alkyl, hydroxy C₁₋₆ alkyl, halogenated C₁₋₆ alkyl and C₁₋₆ alkoxy C₁₋₆ alkyl, or R^{2c} and R^{2d} together with the nitrogen atom to which they are attached, form an 4 to 6-membered heterocycle, The heterocycle contains 0, 1, or 2 additional heteroatoms selected from nitrogen, oxygen, and sulfur as ring members, and wherein m is an integer from 1 to 6;
R³ is aryl or heteroaryl, wherein the aryl or heteroaryl is optionally substituted by one or more R⁸; Each R⁸ is independently halogen, cyano, oxo, C₁₋₆ alkyl optionally substituted by halogen, cyano, hydroxyl or deuterium, C₂-C₆ alkenyl optionally substituted by hydroxyl or deuterium, C₂-C₆ alkynyl optionally substituted by hydroxyl or deuterium, —OR^{8a}, —SR^{8a}, —S(O)₂R^{8a}, —P(═O)R^{8a}R^{8b}, —NR^{8a}R^{8b}, —C(O)NR^{8a}R^{8b}, optionally C₃-C₆ cycloalkyl substituted by halogen or C₁₋₆ alkyl, C₃-C₈ cycloalkyl, heterocyclyl, heteroaryl and aryl, wherein R^{8a} and R^{8b} are each independently hydrogen, C₁₋₆ alkyl substituted by halogen and C₁₋₆ alkoxy-C₁₋₆ alkyl-; or two R⁸ on the same carbon atom form C₃-C₈ cycloalkyl; or R⁸ on two adjacent carbon atoms together with their connected carbon atoms form C₃-C₈ cycloalkyl;
Q¹, Q², and Q³ are each independently N or CR⁶, and M¹ and M² are each independently N or CR⁷, provided that at least one of Q¹ and M¹ is N;
wherein R⁶ and R⁷ are each independently hydrogen, halogen, hydroxyl, cyano, nitro, C₁₋₆ alkyl, C₂₋₆ alkenyl, C₂₋₆ alkynyl, C₃₋₈ cycloalkyl, aryl, heteroaryl or heterocyclyl, —OR^{6a}, —C(O)R^{6a}, —CO₂R^{6a}, —CONR^{6a}R^{6b} or —NR^{6a}R^{6b}, wherein the C₁₋₆ alkyl, C₂₋₆ alkenyl, C₂₋₆ alkynyl, C₃₋₈ cycloalkyl, aryl, heteroaryl and heterocyclyl are independently substituted by one or more of oxo, halogen, hydroxyl, C₁₋₄ alkoxy, C₁₋₄ alkyl, C₃₋₆ cycloalkyl, nitro, cyano and —NR^{d}R^{c}, wherein R^{6a}, R^{6b}, R^{6c} and R^{6d} are each independently hydrogen, C₃₋₆ alkyl, C₁₋₆ alkyl, hydroxy C₁₋₆ alkyl, halogenated C₁₋₆ alkyl and C₁₋₆ alkoxy C₁₋₆ alkyl;
or R⁷ on M² and substituent R⁸ on R³ form a ring structure together with the part to which they are connected; or the substituents on Q² and Q³ are connected to form a saturated or partially saturated 5 to 6-membered aliphatic ring, aromatic ring, or heteroaromatic ring.

2. The compound according to claim 1, wherein 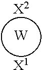
is 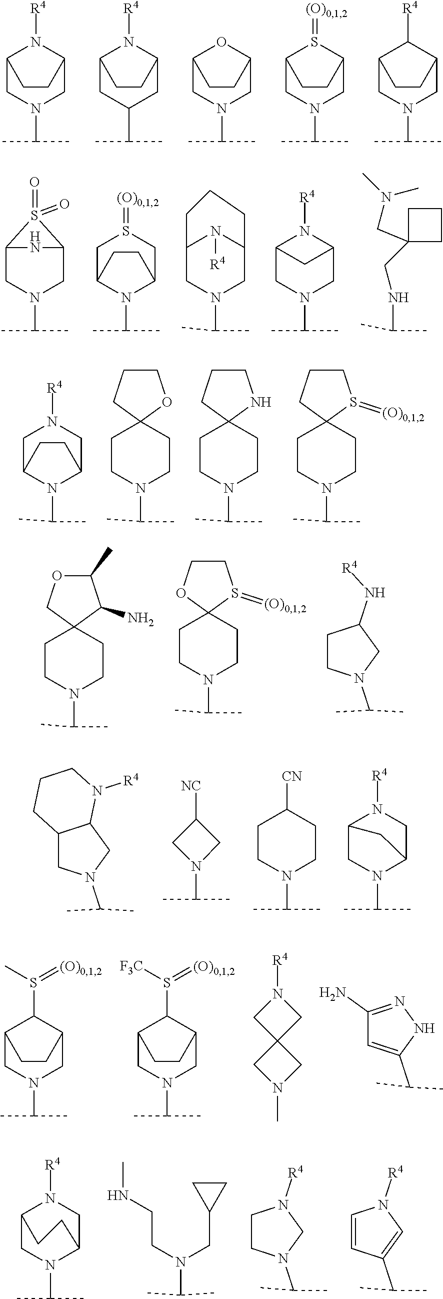 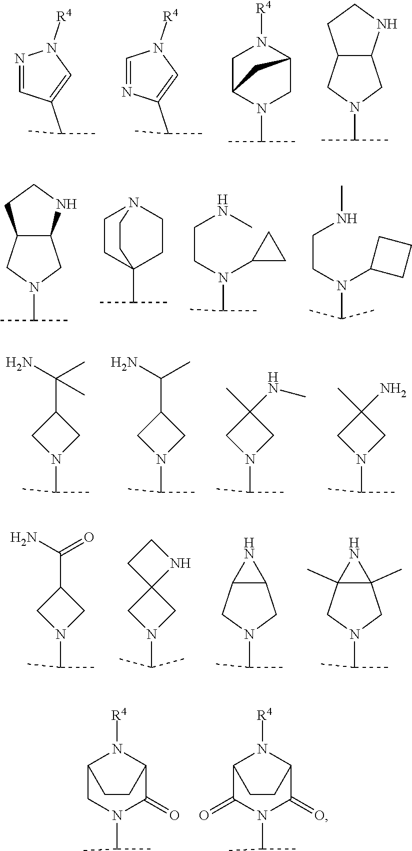
wherein R⁴ is each independently selected from hydrogen, halogen, C₁₋₆ alkyl, CN, —OR^{4a}, —SR^{4a}, —S(O)R^{4a}, —S(O)₂R^{4a}, —C(O)R^{4a}, —C(O)OR^{4a}, —NR^{4a}C(O)R^{4b}, —C(O)NR^{4a}R^{4b} and —S(O)₂N(R^{4a}R^{4b})₂, wherein R^{4a} and R^{4b} are
each independently hydrogen, C₁₋₆ alkyl and hydroxy C₁₋₆ alkyl.

3. The compound according to claim 1, wherein 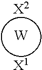
is

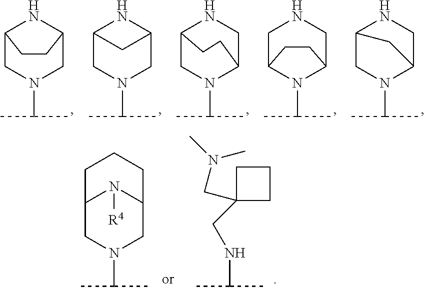

4. The compound according to claim 1, wherein L is —O—CH₂— or —O—.

5. The compound according to claim 1, wherein L is —O—CH₂—, and R² is a heterocyclyl that is unsubstituted or substituted by one or more of halogen, C₁₋₆ alkyl, —OR² and —(CR^{2a}R^{2b})ₘ—OC(O)NR^{2c}R^{2a}, wherein each variable is as defined for formula (I); preferably, the heterocyclyl is unsubstituted or substituted by one or more of halogen, C₁₋₆ alkyl and —OR^{2a}; more preferably, the heterocyclyl is unsubstituted or substituted by one or two of halogen, methyl and methoxy.

6. The compound according to claim 1, wherein L is —O—CH₂—, and R² is a 4 to 8-membered single ring heterocyclyl containing 1, 2, or 3 heteroatoms selected from oxygen, nitrogen, and sulfur as ring members, or a 6 to 12-membered bicyclic heterocyclyl containing 1, 2, or 3 heteroatoms selected from oxygen, nitrogen, and sulfur as ring members, wherein the single ring or bicyclic heterocyclyl is unsubstituted or substituted by one or more of halogen, C₁₋₆ alkyl, OR^{2a} and —(CR^{2a}R^{2b})ₘ—OC(O)NR^{2c}R^{2d}, wherein each variable is defined for formula (I).

7. The compound according to claim 6, wherein the heterocyclyl is unsubstituted or substituted by one or more halogen, C₁₋₆ alkyl, and —OR^{2a}; more preferably, the heterocyclyl is unsubstituted or substituted by one or two halogen, methyl, and methoxy.

8. The compound according to claim 6, wherein L is —O—CH₂—, and R² is a monocyclic heterocyclic ring, which is azetidinyl, pyrrolidyl or piperidyl, the ring is unsubstituted or substituted by one or two halogen or C₁₋₆ alkyl; or L is —O—CH₂—, and R² is a bicyclic heterocycle, which is octahydropentadiene, wherein at least one carbon atom is replaced by a nitrogen atom, and one of the other carbon atoms is optionally replaced by an oxygen atom.

9. The compound according to claim 6, wherein L is —O—CH₂—, and R² is a bicyclic heterocyclic ring, which is tetrahydro-1H-pyrrolizinyl, tetrahydro-1H-furanopyrrolidyl, octahydrocyclopentopyrrolidyl, azabicyclo[3.1.0]hexanyl, wherein the group is unsubstituted or substituted by one or more of halogen, C₁₋₆ alkyl, —OR^{2a} and —(CR^{2a}R^{2b})ₘ—OC(O)NR^{2c}R^{2d}, each of the variables is as defined for formula (I). Preferably substituted by one or more of halogen, C₁₋₆ alkyl and —OR^{2a}; more preferably substituted by one or two of halogen, methyl and methoxy.

10. The compound according to claim 9, wherein the bicyclic heterocyclyl is tetrahydro-1H-pyrrolizin-7-yl, tetrahydro-1H-furano[3,4-b]pyrrole-3a-yl, tetrahydro-1H-furano[3,4-c]pyrrole-3a-yl, tetrahydro-1H-furano[3,4-b]pyrrole-6a-yl, octahydrocyclopenta[b]pyrrole-6a-yl, octahydrocyclopenta[c]pyrrole-3a-yl, octahydrocyclopenta[b]pyrrole-3a-yl, 3-azabicyclic[3.1.0]hexane-1-yl or 2-azabicyclic[3.1.0]hexane-1-yl, wherein the functional group is unsubstituted or substituted by one or more of halogen, C₁₋₆ alkyl, —OR^{2a}, and —(CR^{2a}R^{2b})ₘ—OC(O)NR^{2c}R^{2d}, wherein each variables is as defined for formula (I); preferably substituted by one or more of halogens, C₁₋₆ alkyl, and —OR^{2a}; more preferably, substituted by one or two of halogen, methyl, and methoxy.

11. The compound according to claim 1, wherein L-R² is

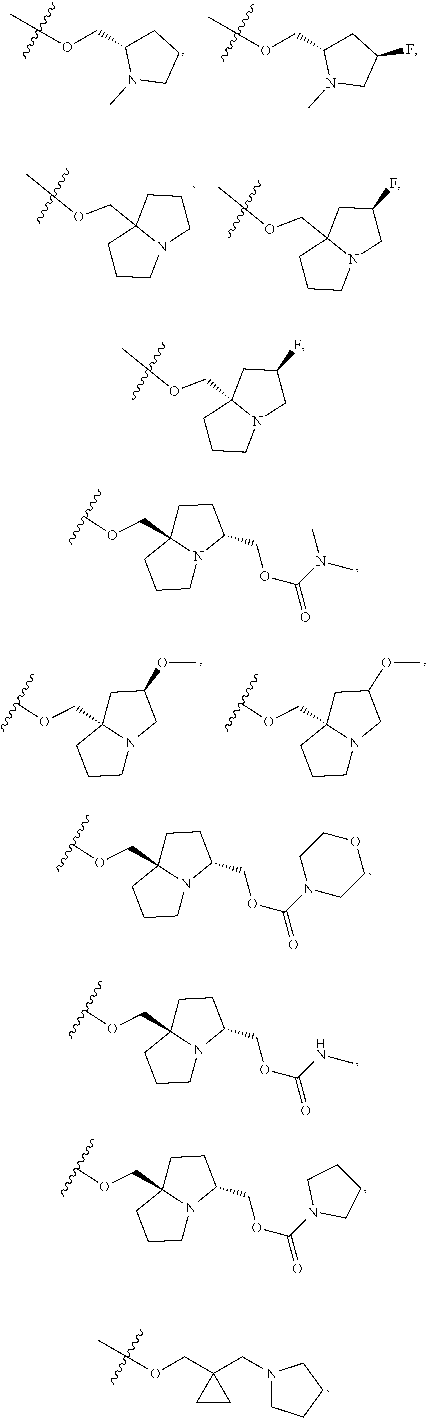

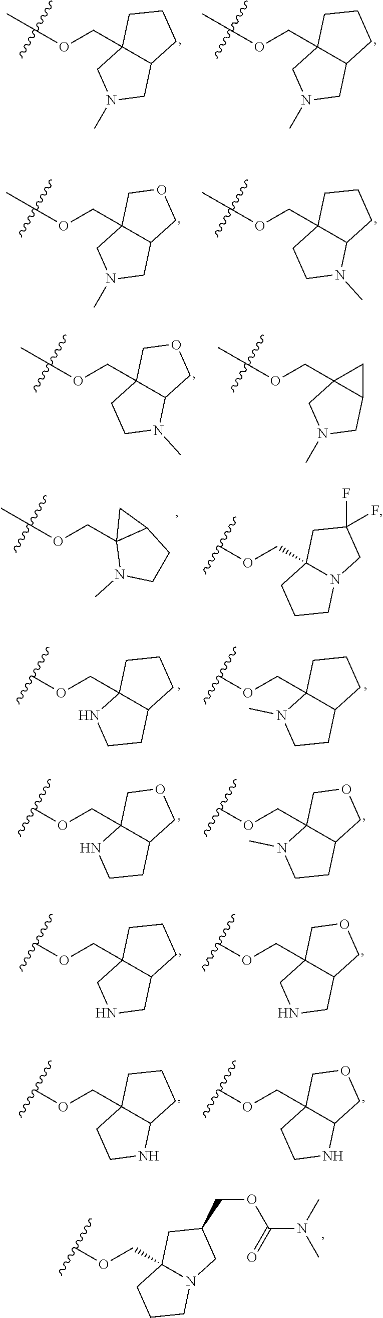

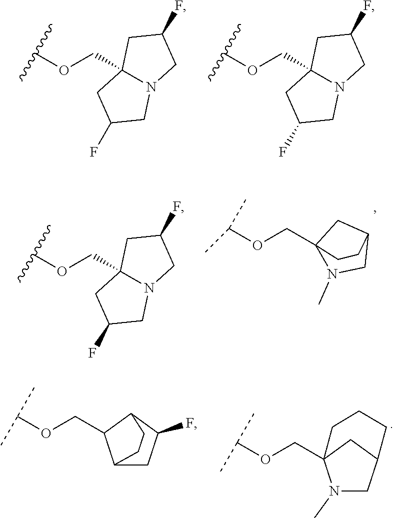

12. The compound according to claim 1, wherein R³ is
a. monocyclic aryl or heteroaryl, selected from phenyl, pyridyl, pyrimidinyl and pyrazinyl;
b. bicyclic aryl or heteroaryl, selected from naphthyl (e.g., naphthalene-1-yl, naphthalene-2-yl, naphthalene-3-yl, naphthalene-4-yl, naphthalene-5-yl, naphthalene-6-yl, naphthalene-7-yl, naphthalene-8-yl), isoquinolinyl (e.g. isoquinoline-1-yl, isoquinoline-3-yl, isoquinoline-4-yl, isoquinoline-5-yl, isoquinoline-6-yl, isoquinoline-7-yl and isoquinoline-8-yl), tetrahydroisoquinoline (e.g., 5,6,7,8-tetrahydroisoquinoline-1-yl, 5,6,7,8-tetrahydroisoquinoline-3-yl, and 5,6,7,8-tetrahydroisoquinoline-4-yl), tetrahydrogenated naphthalene (e.g. 5,6,7,8-tetrahydronaphthalen-1-yl, 5,6,7,8-tetrahydronaphthalen-2-yl, 5,6,7,8-tetrahydronaphthalen-3-yl, 5,6,7,8-tetrahydronaphthalen-4-yl);
c. tricyclic aryl or heteroaryl, selected from 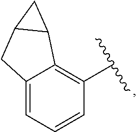 [SMILES: *c1cccc2c1C1CC1C2]
the aryl or heteroaryl is substituted by 1 to 3 R⁸, R⁸ is each independently selected from: halogen, C₁₋₆ alkyl optionally substituted by halogen, C₂-C₆ alkynyl, —P(═O)R^{8a}R^{8b}, —NR^{8a}R^{8b}, —OR^{8a}, —SR^{8a}, C₃-C₆ cycloalkyl optionally substituted by halogen or C₁₋₆ alkyl, wherein R^{8a} and R^{8b} are each independently of hydrogen and C₁₋₆alkyl.

13. The compound according to claim 12, wherein R³ is a naphthalen-1-yl, and the 3 position of the naphthalen-1-yl is unsubstituted or substituted by —OR^{8a}, —P(═O)R^{8a}R^{8b}, or —NR^{8a}R^{8b}, wherein R^{8a} and R^{8b} are each independently hydrogen and C₁₋₆ alkyl.

14. The compound according to claim 13, wherein one or two of the positions 5, 6, 7 and 8 of the naphthalene-1-yl are substituted, the substituent group is independently selected from halogen, C₁₋₆ alkyl optionally substituted by halogen, C₂-C₆ alkynyl, —P(═O)R^{1a}R^{8b}, —NR^{8a}R^{8b}, —OR^{8a}, —SR^{8a}, and C₃-C₆ cycloalkyl optionally substituted by halogen or C₁₋₆ alkyl, wherein R^{8a} and R^{8b} are each independently hydrogen and C₁₋₆alkyl.

15. The compound according to claim 1, wherein R³ is

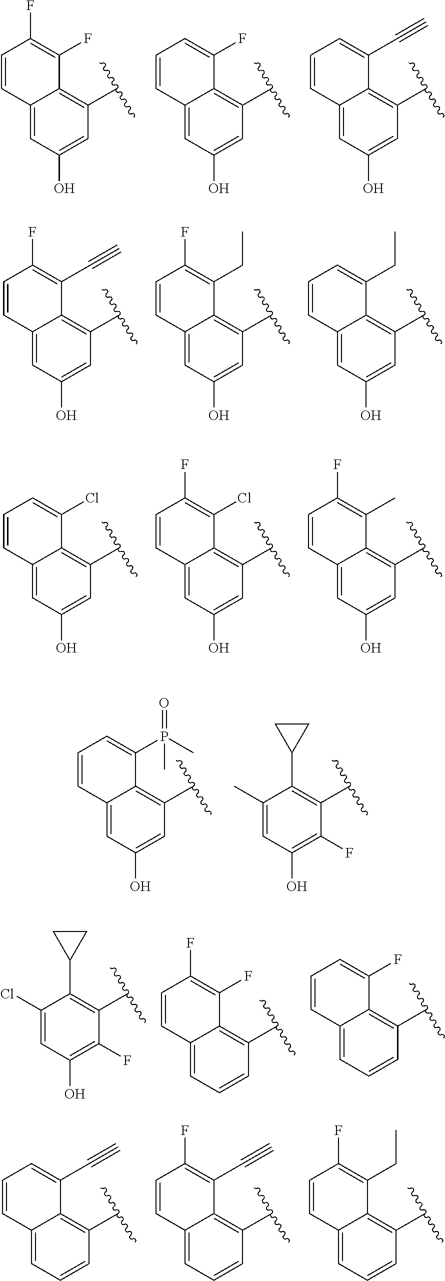

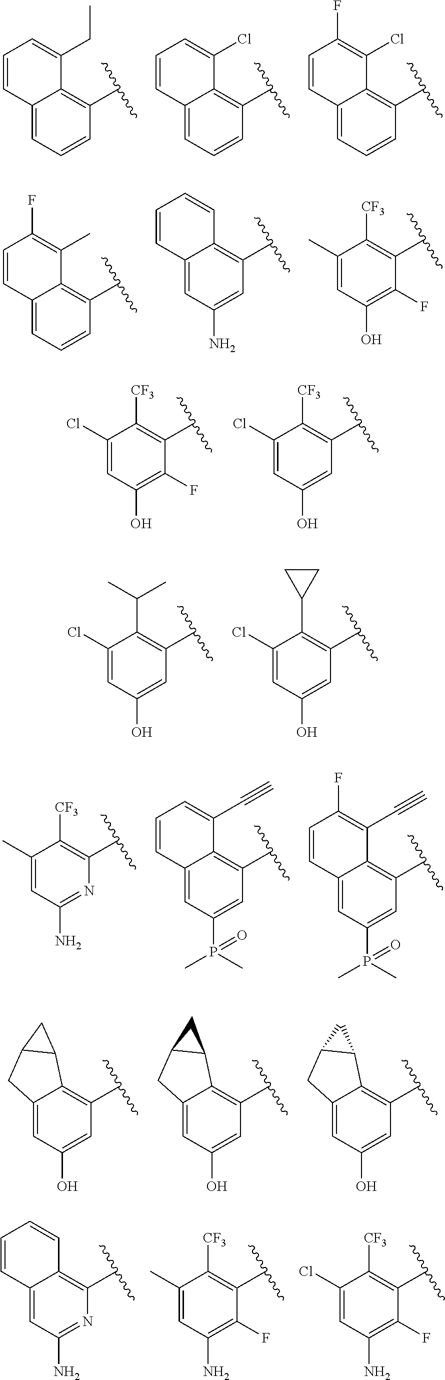

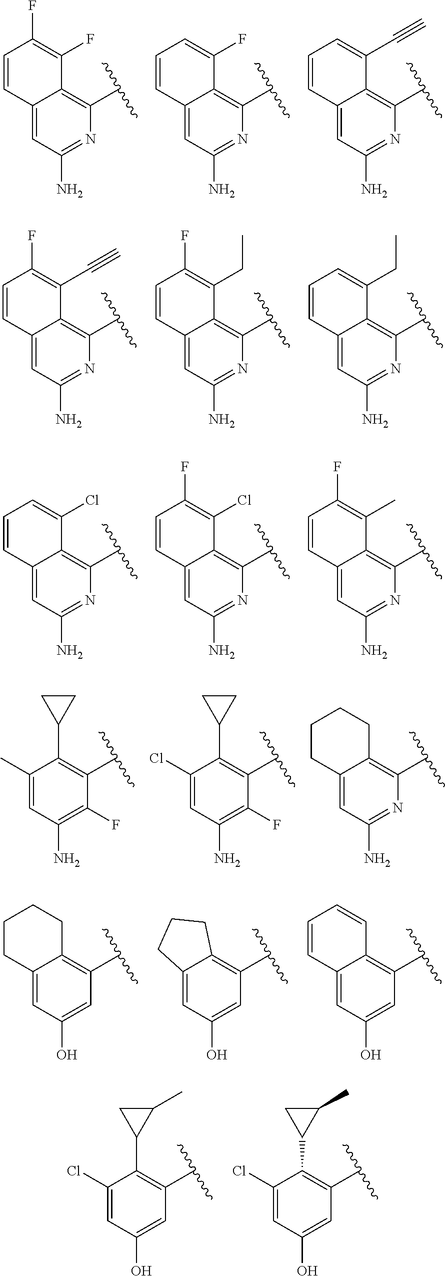

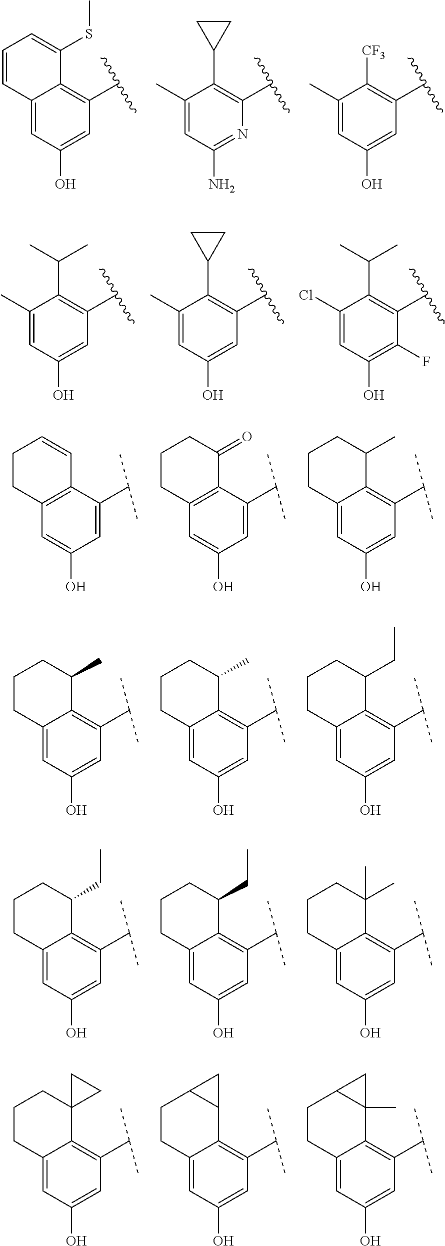

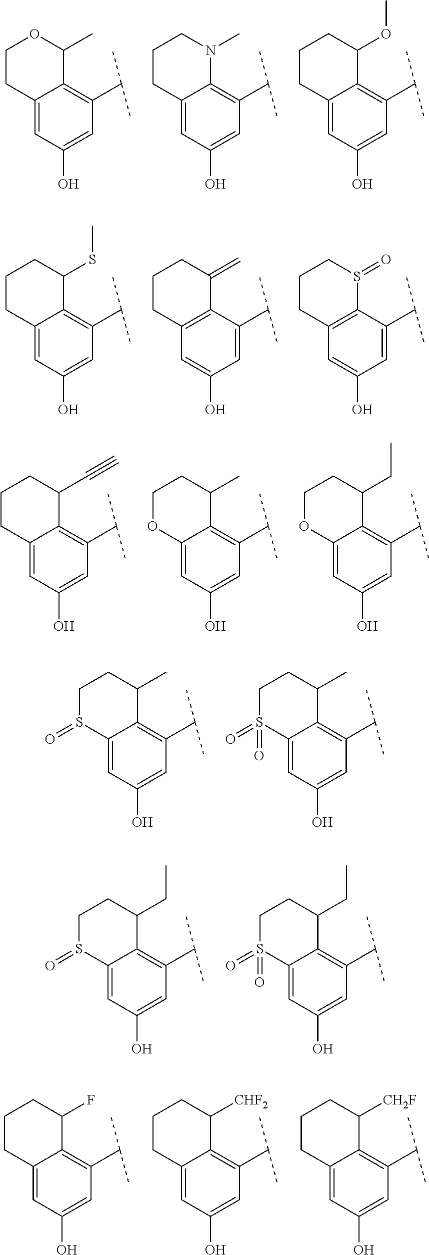

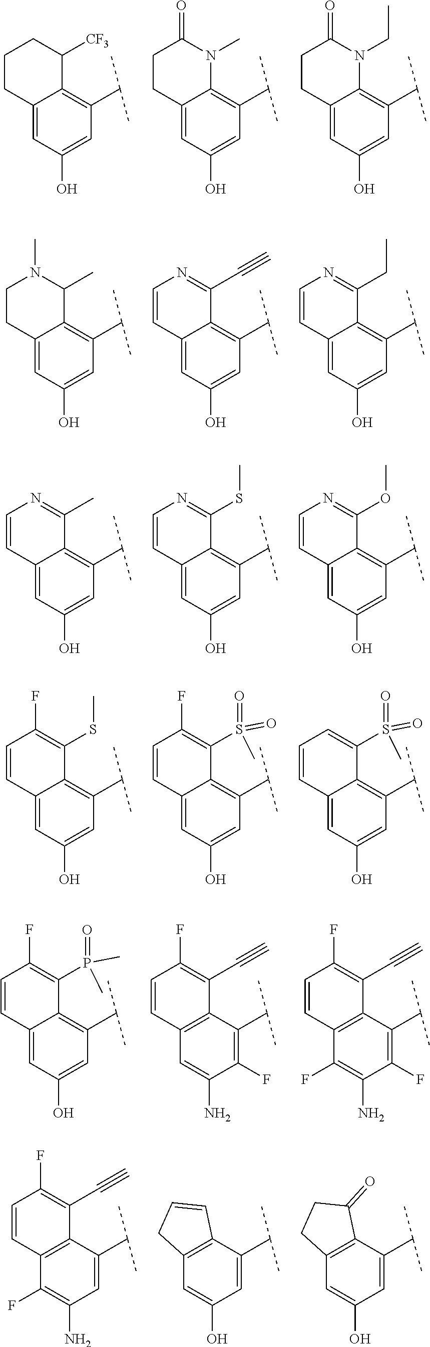

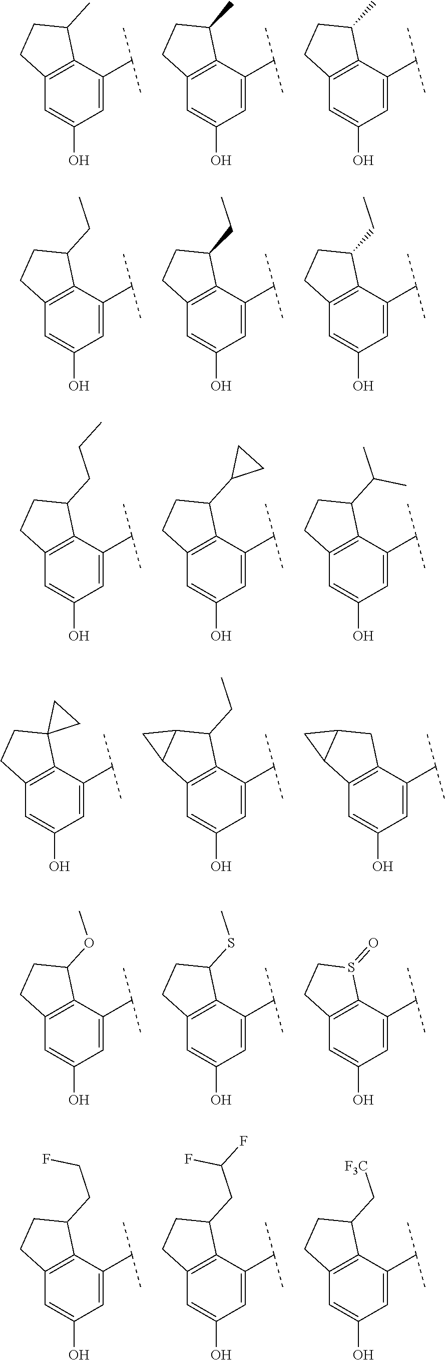

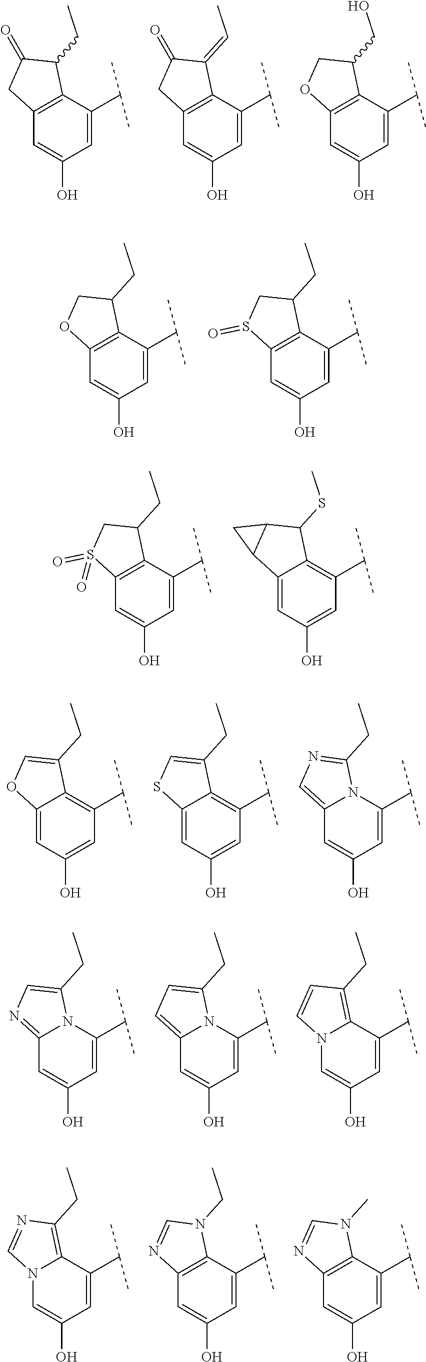

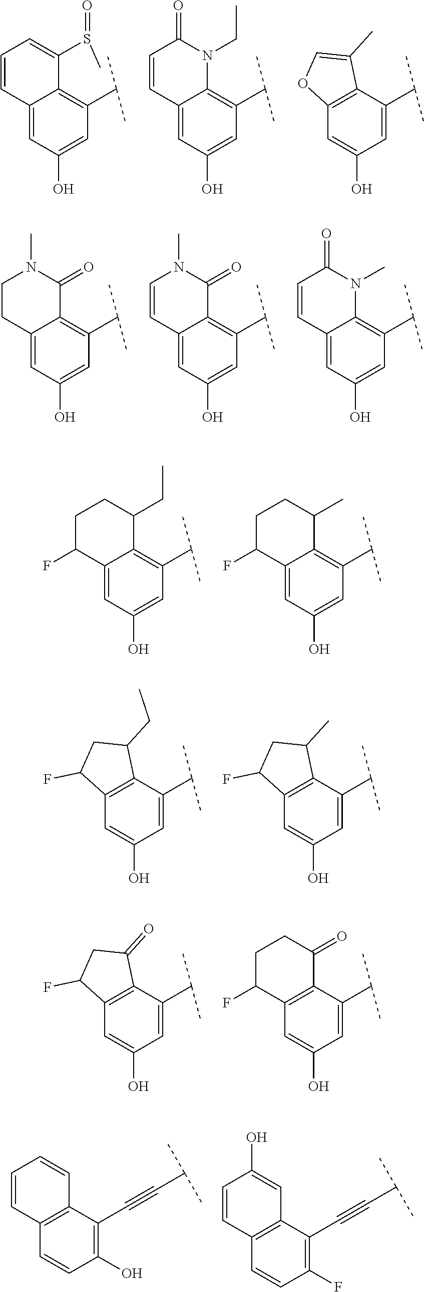

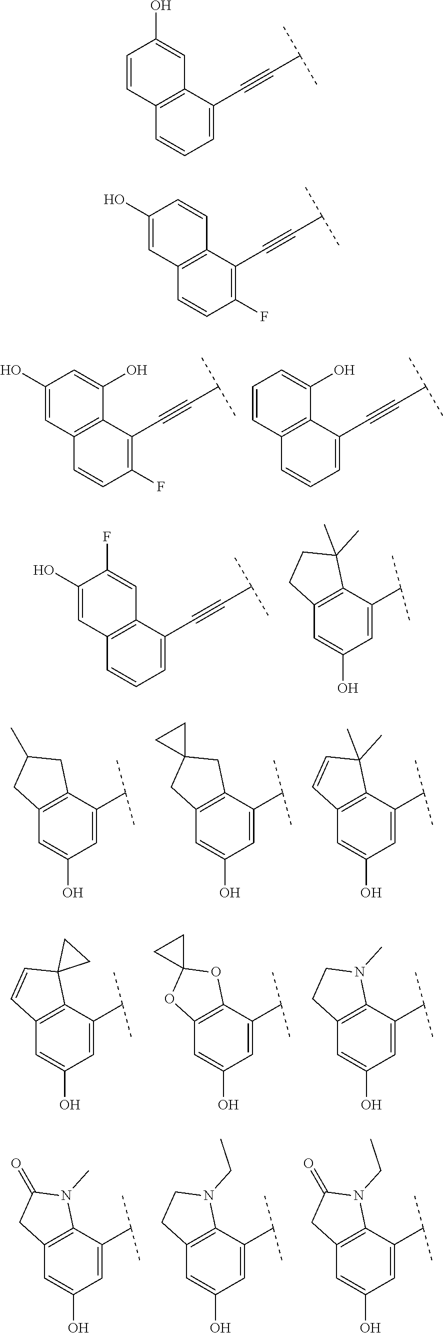

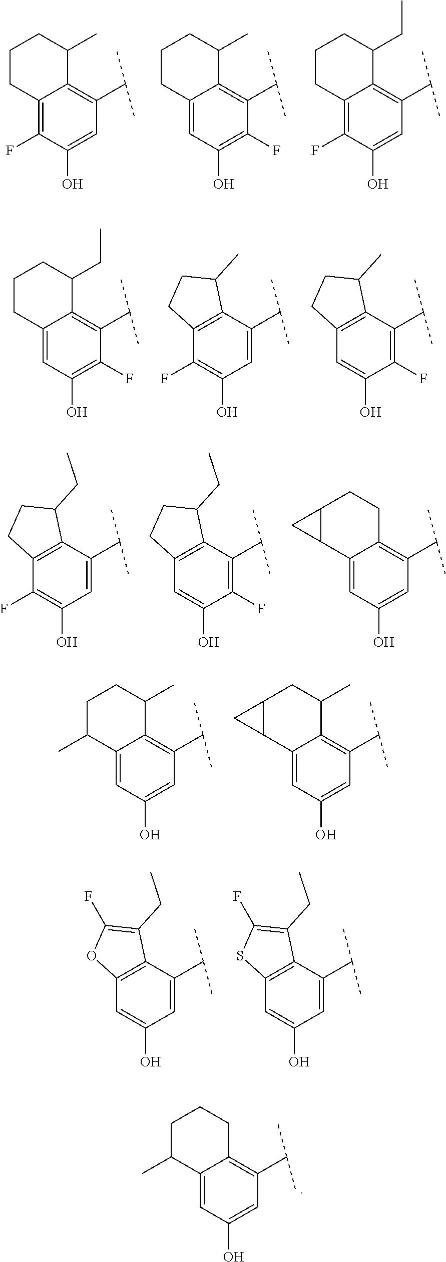

16. The compound according to claim 1, wherein the compound is a specific compound disclosed by the present invention.

17. A pharmaceutical composition comprising the compound or the pharmaceutically acceptable salt, tautomer, prodrug or stereoisomer thereof according to claim 1.

18. A method for treating a cancer associated with H-ras, K-ras or N-ras inhibition, comprising administering a subject in need thereof the compound or the pharmaceutically acceptable salt, tautomer, prodrug or stereoisomer thereof according to claim 1.

19. The method of claim 18, wherein the cancer is a cancer mediated by a G12D mutation.

20. The method of claim 19, wherein the cancer is lung cancer, colorectal cancer or pancreatic cancer.
